(19) United States
(12) Patent Application Publication
(10) Pub. No.: US 2024/0109884 A1
(43) Pub. Date: Apr. 4, 2024

(54) NOVEL COMPOUND AND ORGANIC LIGHT EMITTING DEVICE COMPRISING THE SAME

(71) Applicant: LG CHEM, LTD., Seoul (KR)

(72) Inventors: Minjun KIM, Daejeon (KR); Dong Hoon LEE, Daejeon (KR); Donghee KIM, Daejeon (KR); Jongsoo SONG, Daejeon (KR); Sungju PARK, Daejeon (KR)

(21) Appl. No.: 18/267,913

(22) PCT Filed: Mar. 30, 2022
(86) PCT No.: PCT/KR2022/004527

§ 371 Date: Jun. 16, 2023

(30) Foreign Application Priority Data
Mar. 30, 2021 (KR) 10-2021-0041273
Mar. 30, 2022 (KR) 10-2022-0039623

(51) Int. Cl. C07D 419/14; C07D 417/10; C07D 417/14; C07D 419/10; H10K 85/60

(52) U.S. Cl. CPC C07D 419/14; C07D 417/10; C07D 417/14; C07D 419/10; H10K 85/615; H10K 85/633; H10K 85/654; H10K 85/6574; H10K 85/6576

(57) ABSTRACT

Provided is a compound of Chemical Formula 1:

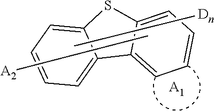

wherein: A₁ represents Chemical Formula 1-a:

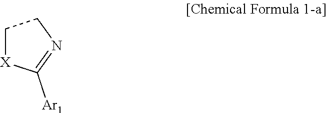

where X is O or S; and Ar₁ is a substituted or unsubstituted C₆₋₆₀ aryl or C₂₋₆₀ heteroaryl containing at least one of N, O and S; and A₂ is Chemical Formula 1-b or Chemical Formula 1-c:

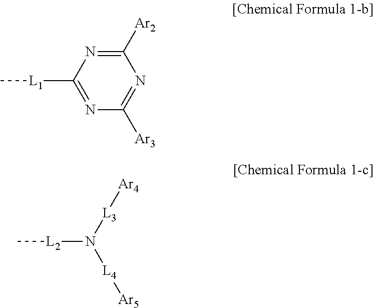

wherein: L₁ to L₄ are each independently a single bond, a substituted or unsubstituted C₆₋₆₀ arylene, or a substituted or unsubstituted C₂₋₆₀ heteroarylene containing at least one of N, O and S; Ar₂ to Ar₅ are each independently a substituted or unsubstituted C₆₋₆₀ aryl or C₂₋₆₀ heteroaryl containing at least one of N, O and S; D is deuterium; and n is an integer of 0 to 5, and an organic light emitting device including the same. An organic light-emitting device containing the compound exhibits improved efficiency, driving voltage and/or lifespan characteristics.

### CROSS-REFERENCE TO RELATED APPLICATION(S)

This application is a National Stage Application of International Application No. PCT/KR2022/004527 filed on Mar. 30, 2022, which claims the benefit of Korean Patent Applications No. 10-2021-0041273 filed on Mar. 30, 2021 and No. 10-2022-0039623 filed on Mar. 30, 2022 in the Korean Intellectual Property Office, the disclosures of which are incorporated herein by reference in their entirety.

### TECHNICAL FIELD

The present disclosure relates to a novel compound and an organic light emitting device including the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of new materials for the organic materials used in the organic light emitting devices as described above.

### PRIOR ART LITERATURE

(Patent Literature 0001) Korean Unexamined Patent Publication No. 10-2000-0051826

### BRIEF DESCRIPTION

### Technical Problem

The present disclosure relates to a novel compound and an organic light emitting device including the same.

### Technical Solution

In the present disclosure, provided is a compound of Chemical Formula 1:

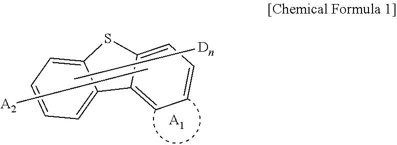

wherein in the Chemical Formula 1:
A₁ represents Chemical Formula 1-a:

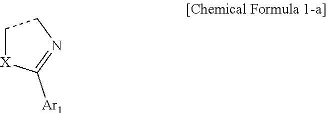

wherein in the Chemical Formula 1-a:
the dotted line is fused with an adjacent ring;
X is O or S;
Ar₁ is a substituted or unsubstituted C₆₋₆₀ aryl, or a substituted or unsubstituted C₂₋₆₀ heteroaryl containing at least one heteroatom selected from the group consisting of N, O and S; and
A₂ is a substituent of the following Chemical Formula 1-b or the following Chemical Formula 1-c:

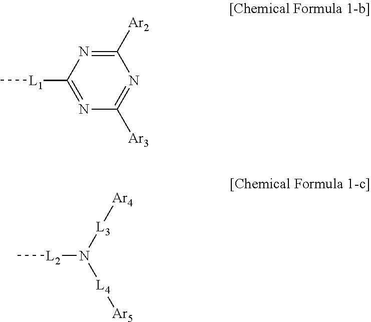

wherein in the Chemical Formulae 1-b and 1-c:
L₁ to L₄ are each independently a single bond, a substituted or unsubstituted C₆₋₆₀ arylene, or a substituted or unsubstituted C₂₋₆₀ heteroarylene containing at least one heteroatom selected from the group consisting of N, O and S;
Ar₂ to Ar₅ are each independently a substituted or unsubstituted C₆₋₆₀ aryl, or a substituted or unsubstituted C₂₋₆₀ heteroaryl containing at least one heteroatom selected from the group consisting of N, O and S;
D is deuterium; and
n is an integer of 0 to 5.

In addition, provided is an organic light emitting device including: a first electrode; a second electrode that is opposite to the first electrode; and one or more organic material layers that are between the first electrode and the second electrode, wherein at least one layer of the one or more organic material layers includes the compound of Chemical Formula 1.

### Advantageous Effects

The compound of Chemical Formula 1 can be used as a material for an organic material layer of an organic light emitting device, and can improve efficiency, low driving voltage, and/or lifespan of the organic light emitting device. In particular, the compound of Chemical Formula 1 can be used as a material for hole injection, hole transport, hole injection and transport, electron blocking, light emission, electron transport, or electron injection.

### BRIEF DESCRIPTION OF THE DRAWINGS

FIG. 1 shows an example of an organic light emitting device including a substrate 1, an anode 2, an organic material layer 3, and a cathode 4.

FIG. 2 shows an example of an organic light emitting device including a substrate 1, an anode 2, a hole injection layer 5, a hole transport layer 6, an electron blocking layer 7, a light emitting layer 8, a hole blocking layer 9, an electron transport layer 10, an electron injection layer 11, and a cathode 4.

FIG. 3 shows an example of an organic light emitting device including a substrate 1, an anode 2, a hole injection layer 5, a hole transport layer 6, an electron blocking layer 7, a light emitting layer 8, a hole blocking layer 9, an electron injection and transport layer 12, and a cathode 4.

### DETAILED DESCRIPTION

Hereinafter, embodiments of the present disclosure will be described in more detail to facilitate understanding of the invention.

In the present disclosure, provided is a compound of Chemical Formula 1.

As used herein, the notation

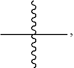

and [symbol] mean a bond linked to another substituent group.

As used herein, the term “substituted or unsubstituted” means being unsubstituted or substituted with one or more substituents selected from the group consisting of deuterium, a halogen group, a nitrile group, a nitro group, a hydroxyl group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heterocyclic group containing at least one of N, O and S atoms, or being unsubstituted or substituted with a substituent in which two or more substituents of the above-exemplified substituents are connected. For example, “a substituent in which two or more substituents are connected” can be a biphenyl group. Namely, a biphenyl group can be an aryl group, or it can also be interpreted as a substituent in which two phenyl groups are connected.

In the present disclosure, the carbon number of a carbonyl group is not particularly limited, but is preferably 1 to 40. Specifically, the carbonyl group can be a compound having the following structural formulae, but is not limited thereto:

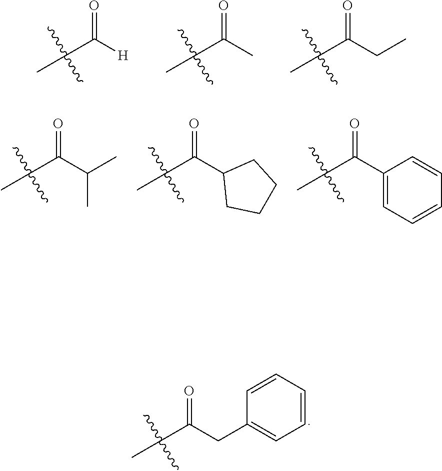

In the present disclosure, an ester group can have a structure in which oxygen of the ester group is substituted by a straight-chain, branched-chain, or cyclic alkyl group having 1 to 25 carbon atoms, or an aryl group having 6 to 25 carbon atoms. Specifically, the ester group can be a compound having the following structural formulae, but is not limited thereto:

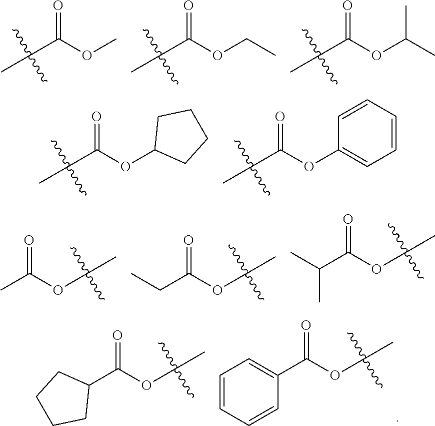

In the present disclosure, the carbon number of an imide group is not particularly limited, but is preferably 1 to 25. Specifically, the imide group can be a compound having the following structural formulae, but is not limited thereto:

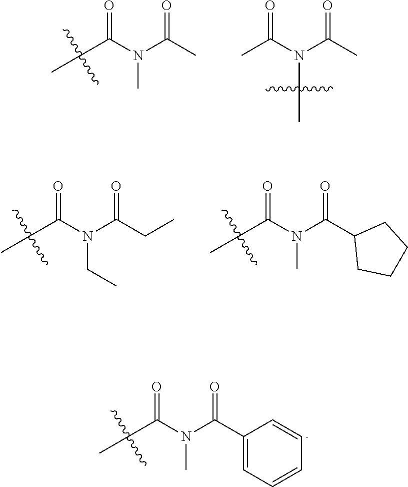

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, a phenylboron group, and the like, but is not limited thereto.

In the present disclosure, examples of a halogen group include fluorine, chlorine, bromine, or iodine.

In the present disclosure, the alkyl group can be straight-chain, or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The monocyclic aryl group includes a phenyl group, a biphenyl group, a terphenyl group and the like, but is not limited thereto. The polycyclic aryl group includes a naphthyl group, an anthracenyl group, a phenanthryl group, a pyrenyl group, a perylenyl group, a chrysenyl group, a fluorenyl group or the like, but is not limited thereto.

In the present disclosure, a fluorenyl group can be substituted, and two substituents can be bonded to each other to form a spiro structure. In the case where the fluorenyl group is substituted,

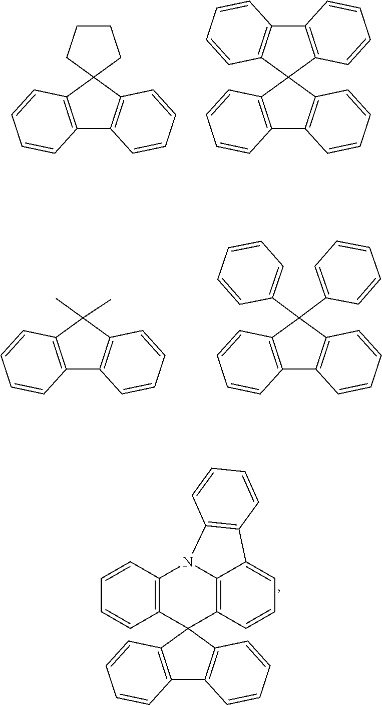

and the like can be formed. However, the structure is not limited thereto.

In the present disclosure, a heterocyclic group is a heterocyclic group containing at least one heteroatom of O, N, Si and S as a heterogeneous element, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can apply the aforementioned description of the heterocyclic group. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heterocyclic group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocycle is not a monovalent group but formed by combining two substituent groups.

The compound of Chemical Formula 1 has a core in which a benzoxazole or benzothiazole ring is fused to a benzothiophene ring, and includes a triazine or amine substituent bonded thereto. As the above structure is satisfied, the compound of Chemical Formula 1 exhibits a low voltage when applied to an organic light emitting device, and has excellent efficiency and lifespan.

The compound of Chemical Formula 1 can be a compound of any one of the following Chemical Formulae 1-1 to 1-4:

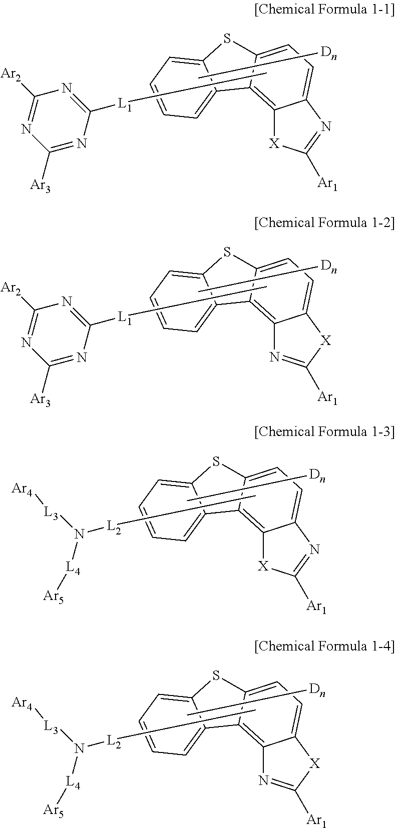

in the Chemical Formulae 1-1 to 1-4,
X, L₁ to L₄, Ar₁ to Ar₅, D, and n are as defined in Chemical Formula 1.

Preferably, L₁ and L₂ are each independently a single bond, or a substituted or unsubstituted C₆₋₂₀ arylene. More preferably, L₁ and L₂ are each independently a single bond, phenylene, biphenyldiyl, or naphthalenediyl.

Preferably, L₃ and L₄ are each independently a single bond, or a substituted or unsubstituted C₆₋₂₀ arylene. More preferably, L₃ and L₄ are each independently a single bond, phenylene, biphenyldiyl, or naphthalenediyl.

Preferably, Ar₁ is a substituted or unsubstituted C₆₋₂₀ aryl, or a substituted or unsubstituted C₂₋₂₀ heteroaryl containing at least one heteroatom selected from the group consisting of N, O and S.

More preferably, Ar₁ is phenyl, biphenylyl, naphthyl, dibenzofuranyl, or dibenzothiophenyl.

Preferably, Ar₂ to Ar₅ are each independently a substituted or unsubstituted C₆₋₂₀ aryl, or a substituted or unsubstituted C₂₋₂₀ heteroaryl containing at least one heteroatom selected from the group consisting of N, O and S.

Preferably, Ar₂ and Ar₃ are each independently phenyl, biphenylyl, naphthyl, phenylnaphthyl (i.e., naphthyl substituted with one phenyl), naphthylphenyl (i.e., phenyl substituted with one naphthyl), phenanthrenylphenyl (i.e., phenyl substituted with one phenanthrenyl), dibenzofuranyl, dibenzothiophenyl, or phenanthrenyl.

Preferably, Ar₄ and Ar₅ are each independently phenyl, biphenylyl, terphenylyl, naphthyl, naphthylphenyl, phenylnaphthyl, phenanthrenyl, 9-phenylcarbazolyl, dibenzofuranyl, or dibenzothiophenyl.

Meanwhile, in the compound of Chemical Formula 1, at least one hydrogen can be substituted with deuterium. That is, n in Chemical Formula 1 can be an integer of 1 or more, and/or at least one substituent of L₁ to L₄ and Ar₁ to Ar₅ in Chemical Formula 1 can be substituted with deuterium.

Representative examples of the compound of Chemical Formula 1 are as follows:

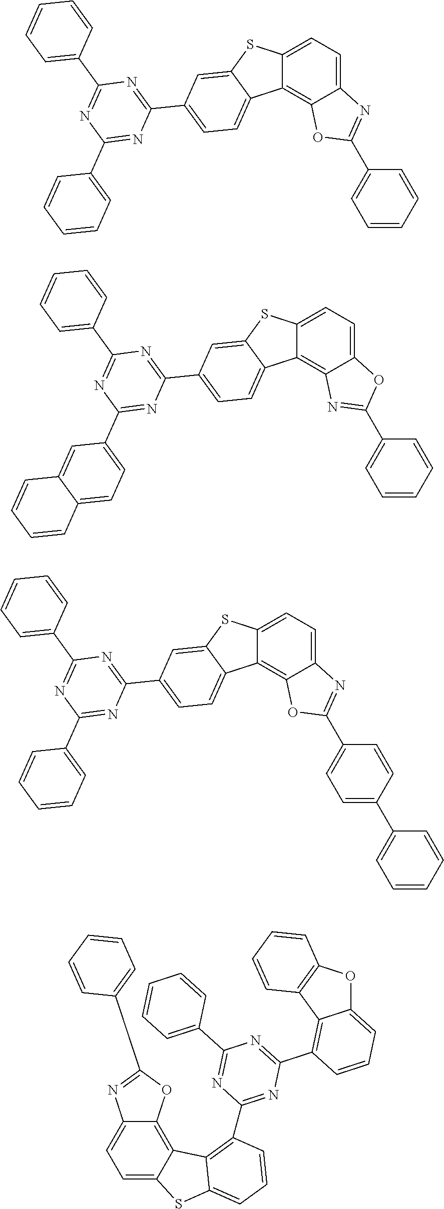

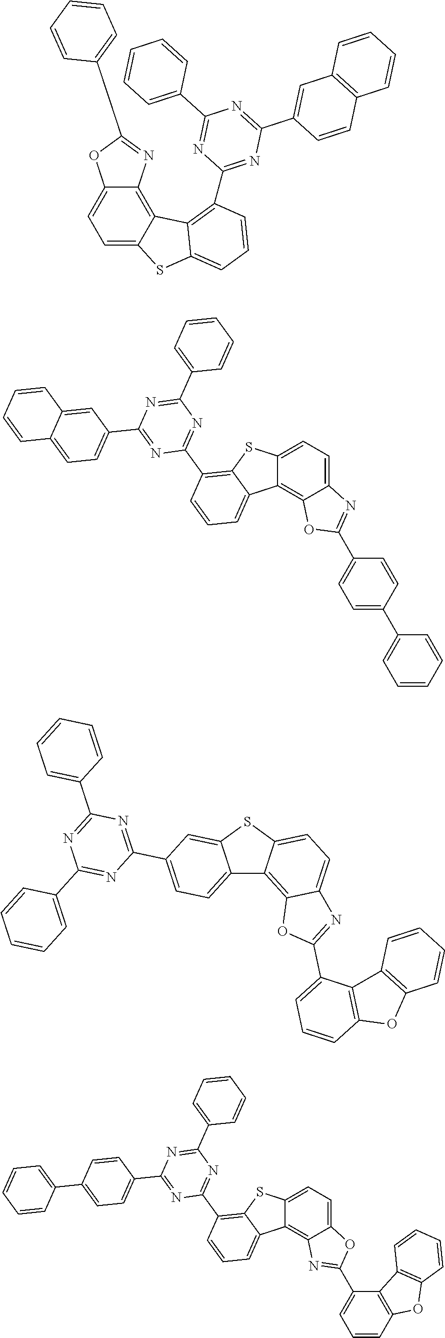 [SMILES: c1ccc(-c2nc(-c3ccc4ccccc4c3)nc(-c3cccc4sc5ccc6oc(-c7ccccc7)nc6c5c34)n2)cc1 ; c1ccc(-c2ccc(-c3nc4ccc5sc6c(-c7nc(-c8ccccc8)nc(-c8ccc9ccccc9c8)n7)cccc6c5c4o3)cc2)cc1 ; c1ccc(-c2nc(-c3ccccc3)nc(-c3ccc4c(c3)sc3ccc5nc(-c6cccc7oc8ccccc8c67)oc5c34)n2)cc1 ; c1ccc(-c2ccc(-c3nc(-c4ccccc4)nc(-c4cccc5c4sc4ccc6oc(-c7cccc8oc9ccccc9c78)nc6c45)n3)cc2)cc1]

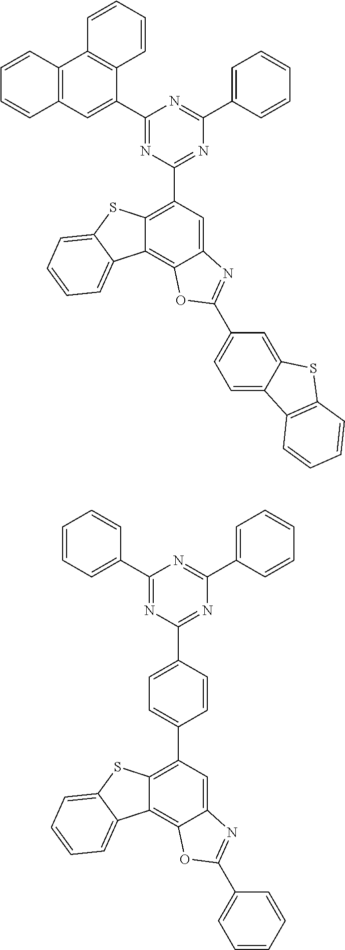 [SMILES: c1ccc(-c2nc(-c3cc4ccccc4c4ccccc34)nc(-c3cc4nc(-c5ccc6c(c5)sc5ccccc56)oc4c4c3sc3ccccc34)n2)cc1 ; c1ccc(-c2nc(-c3ccccc3)nc(-c3ccc(-c4cc5nc(-c6ccccc6)oc5c5c4sc4ccccc45)cc3)n2)cc1]

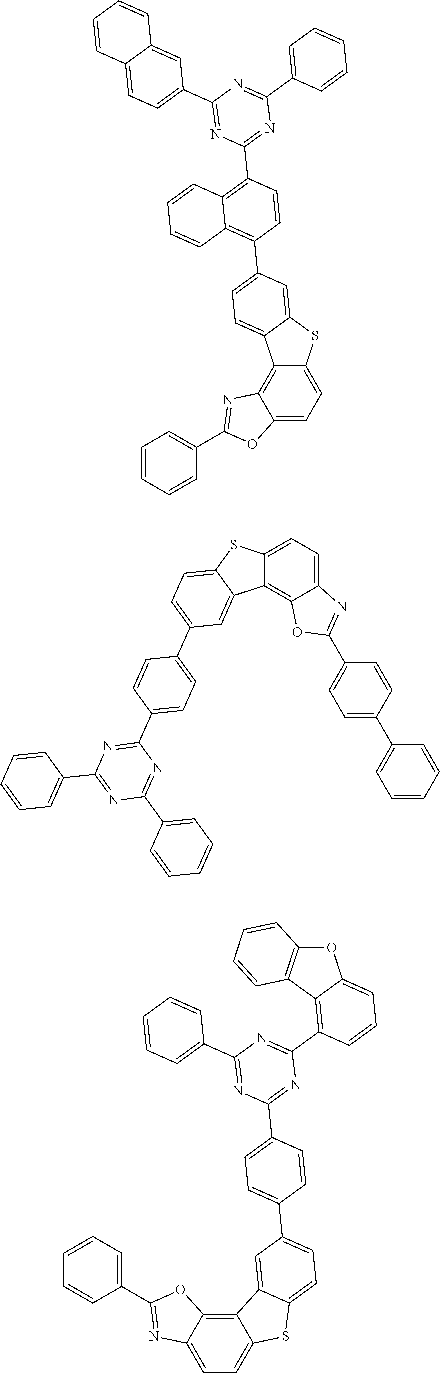 [SMILES: c1ccc(-c2nc(-c3ccc4ccccc4c3)nc(-c3ccc(-c4ccc5c(c4)sc4ccc6oc(-c7ccccc7)nc6c45)c4ccccc34)n2)cc1 ; c1ccc(-c2ccc(-c3nc4ccc5sc6ccc(-c7ccc(-c8nc(-c9ccccc9)nc(-c9ccccc9)n8)cc7)cc6c5c4o3)cc2)cc1 ; c1ccc(-c2nc(-c3ccc(-c4ccc5sc6ccc7nc(-c8ccccc8)oc7c6c5c4)cc3)nc(-c3cccc4oc5ccccc5c34)n2)cc1]

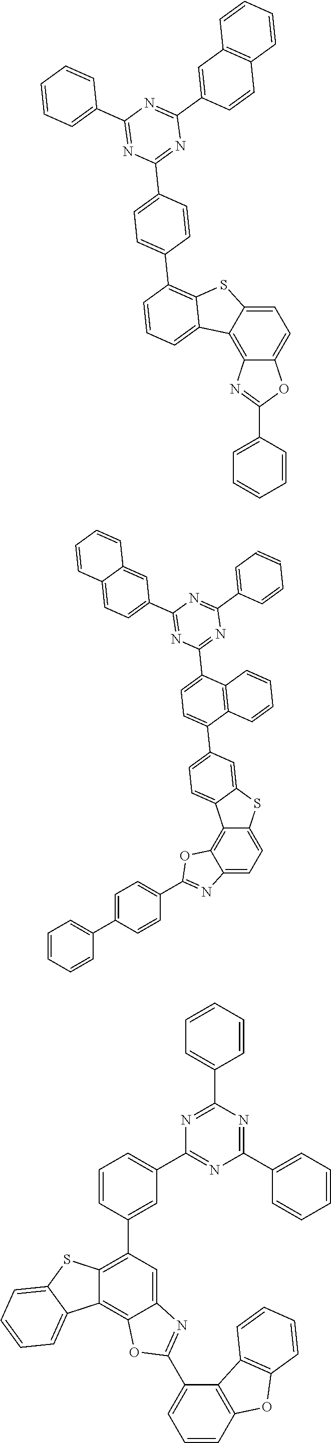 [SMILES: c1ccc(-c2nc(-c3ccc(-c4cccc5c4sc4ccc6oc(-c7ccccc7)nc6c45)cc3)nc(-c3ccc4ccccc4c3)n2)cc1 ; c1ccc(-c2ccc(-c3nc4ccc5sc6cc(-c7ccc(-c8nc(-c9ccccc9)nc(-c9ccc%10ccccc%10c9)n8)c8ccccc78)ccc6c5c4o3)cc2)cc1 ; c1ccc(-c2nc(-c3ccccc3)nc(-c3cccc(-c4cc5nc(-c6cccc7oc8ccccc8c67)oc5c5c4sc4ccccc45)c3)n2)cc1]

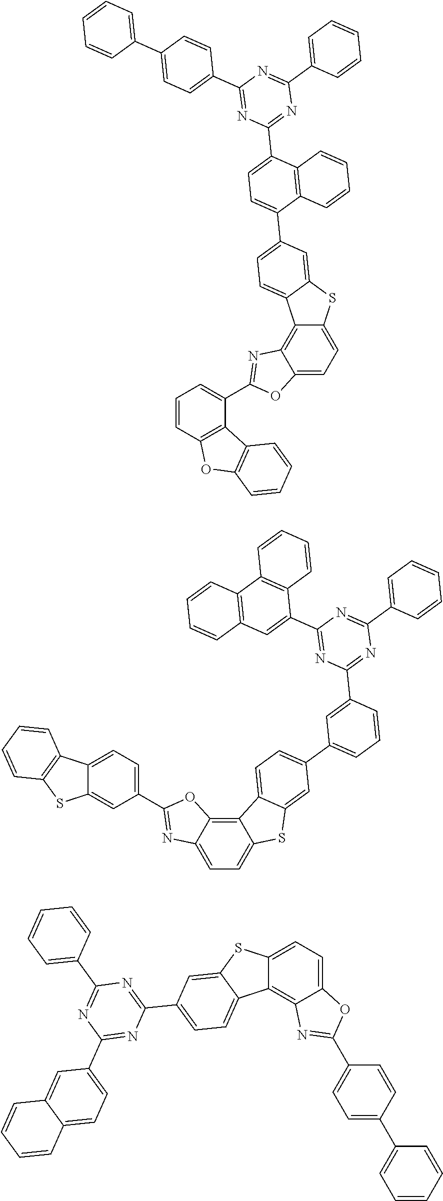

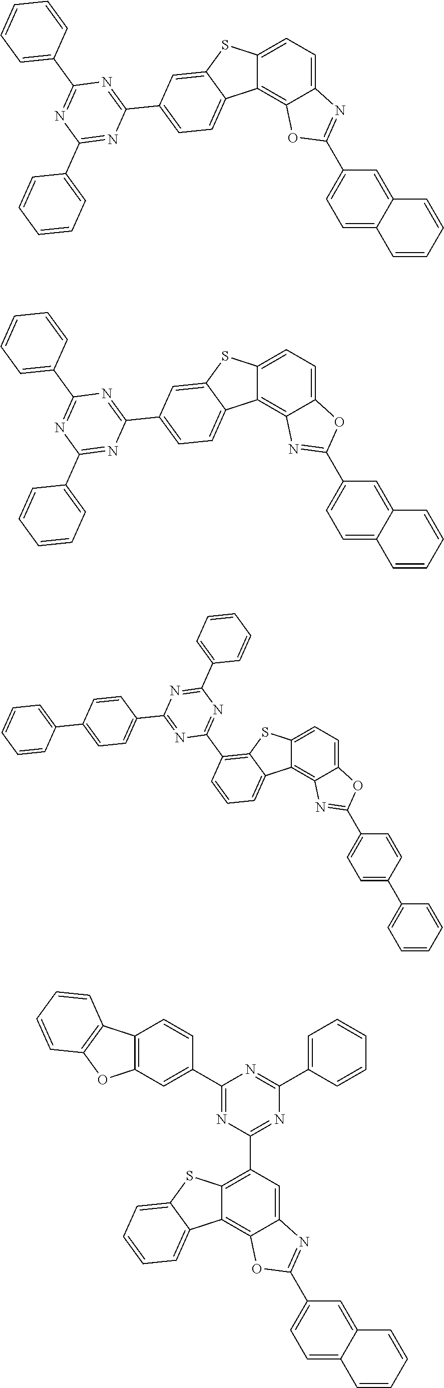 [SMILES: c1ccc(-c2nc(-c3ccccc3)nc(-c3ccc4c(c3)sc3ccc5nc(-c6ccc7ccccc7c6)oc5c34)n2)cc1 ; c1ccc(-c2nc(-c3ccccc3)nc(-c3ccc4c(c3)sc3ccc5oc(-c6ccc7ccccc7c6)nc5c34)n2)cc1 ; c1ccc(-c2ccc(-c3nc(-c4ccccc4)nc(-c4cccc5c4sc4ccc6oc(-c7ccc(-c8ccccc8)cc7)nc6c45)n3)cc2)cc1 ; c1ccc(-c2nc(-c3ccc4c(c3)oc3ccccc34)nc(-c3cc4nc(-c5ccc6ccccc6c5)oc4c4c3sc3ccccc34)n2)cc1]

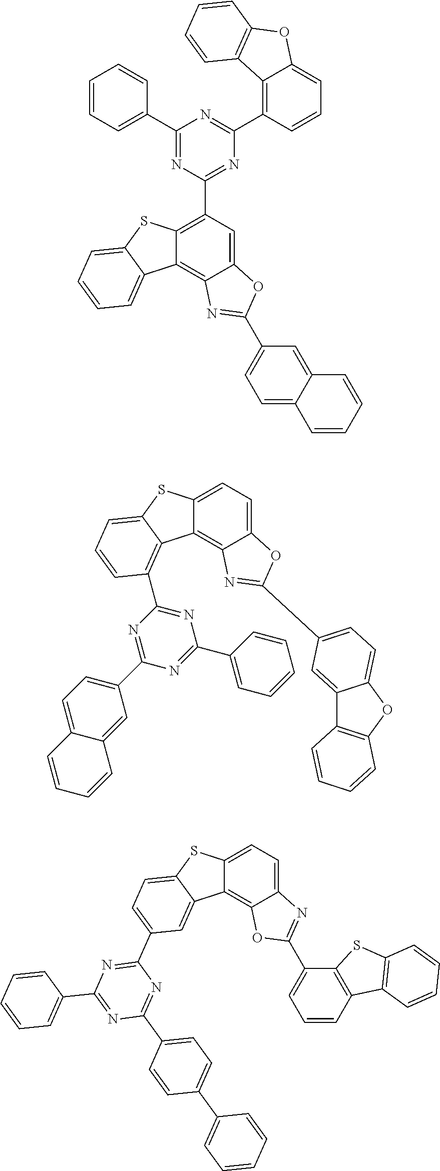 [SMILES: c1ccc(-c2nc(-c3cc4oc(-c5ccc6ccccc6c5)nc4c4c3sc3ccccc34)nc(-c3cccc4oc5ccccc5c34)n2)cc1 ; c1ccc(-c2nc(-c3ccc4ccccc4c3)nc(-c3cccc4sc5ccc6oc(-c7ccc8oc9ccccc9c8c7)nc6c5c34)n2)cc1 ; c1ccc(-c2ccc(-c3nc(-c4ccccc4)nc(-c4ccc5sc6ccc7nc(-c8cccc9c8sc8ccccc89)oc7c6c5c4)n3)cc2)cc1]

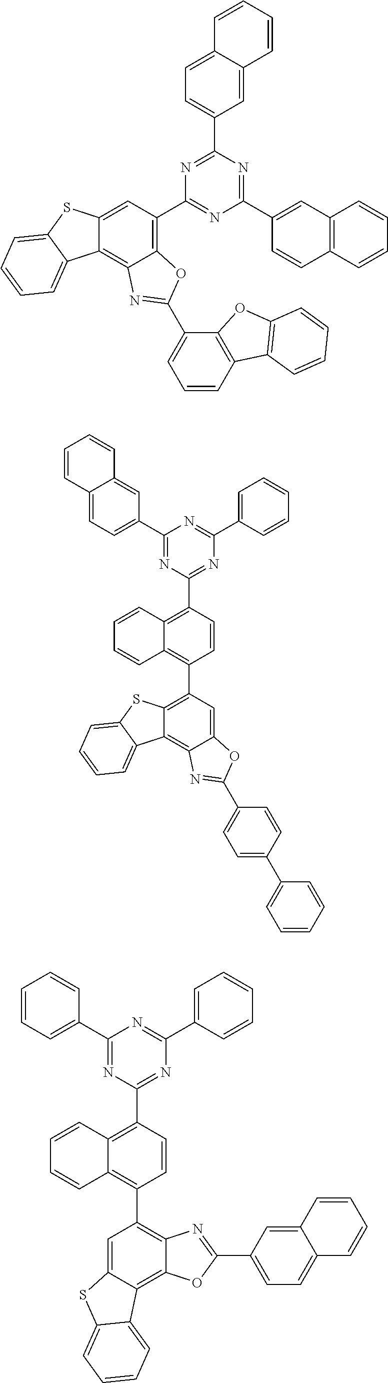 [SMILES: c1ccc2cc(-c3nc(-c4ccc5ccccc5c4)nc(-c4cc5sc6ccccc6c5c5nc(-c6cccc7c6oc6ccccc67)oc45)n3)ccc2c1 ; c1ccc(-c2ccc(-c3nc4c(cc(-c5ccc(-c6nc(-c7ccccc7)nc(-c7ccc8ccccc8c7)n6)c6ccccc56)c5sc6ccccc6c54)o3)cc2)cc1 ; c1ccc(-c2nc(-c3ccccc3)nc(-c3ccc(-c4cc5sc6ccccc6c5c5oc(-c6ccc7ccccc7c6)nc45)c4ccccc34)n2)cc1]

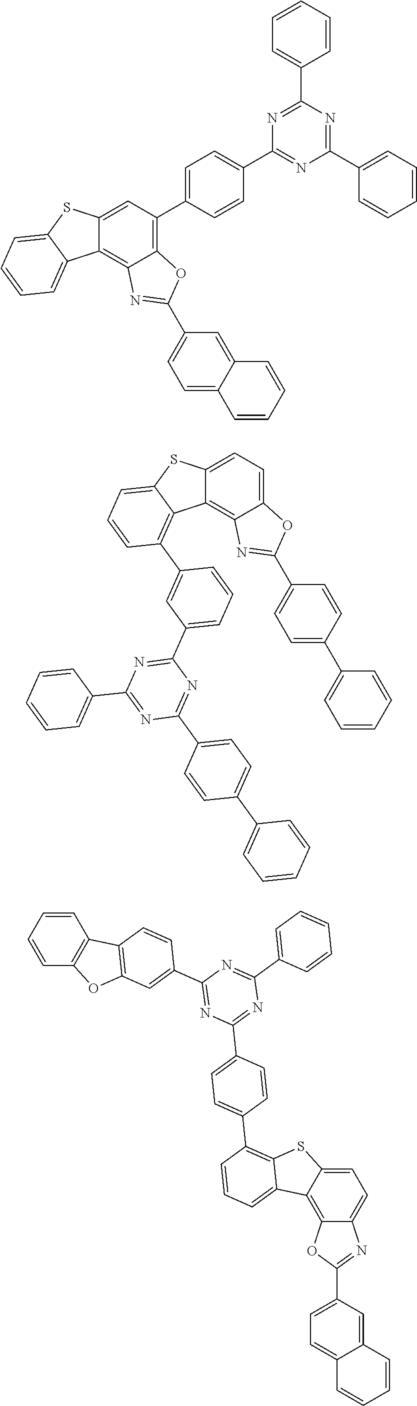

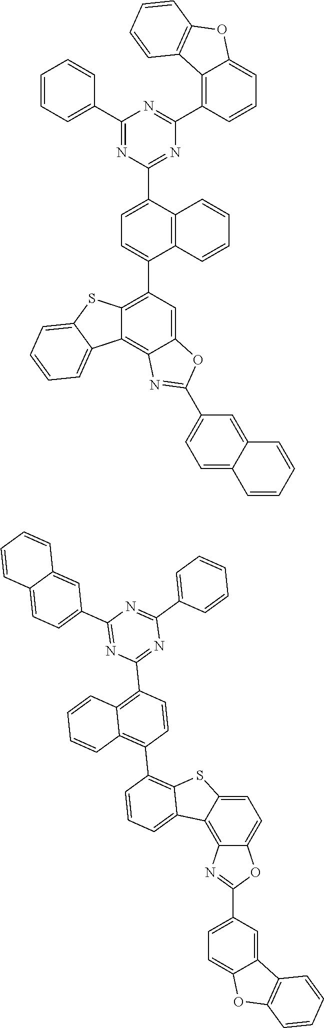 [SMILES: c1ccc(-c2nc(-c3ccc(-c4cc5oc(-c6ccc7ccccc7c6)nc5c5c4sc4ccccc45)c4ccccc34)nc(-c3cccc4oc5ccccc5c34)n2)cc1 ; c1ccc(-c2nc(-c3ccc4ccccc4c3)nc(-c3ccc(-c4cccc5c4sc4ccc6oc(-c7ccc8oc9ccccc9c8c7)nc6c45)c4ccccc34)n2)cc1]

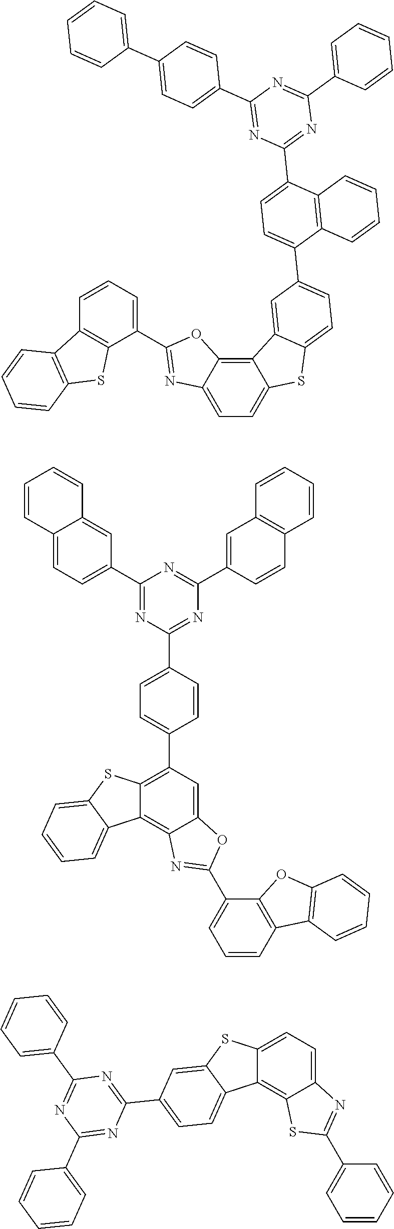 [SMILES: c1ccc(-c2ccc(-c3nc(-c4ccccc4)nc(-c4ccc(-c5ccc6sc7ccc8nc(-c9cccc%10c9sc9ccccc9%10)oc8c7c6c5)c5ccccc45)n3)cc2)cc1 ; c1ccc2cc(-c3nc(-c4ccc(-c5cc6oc(-c7cccc8c7oc7ccccc78)nc6c6c5sc5ccccc56)cc4)nc(-c4ccc5ccccc5c4)n3)ccc2c1 ; c1ccc(-c2nc(-c3ccccc3)nc(-c3ccc4c(c3)sc3ccc5nc(-c6ccccc6)sc5c34)n2)cc1]

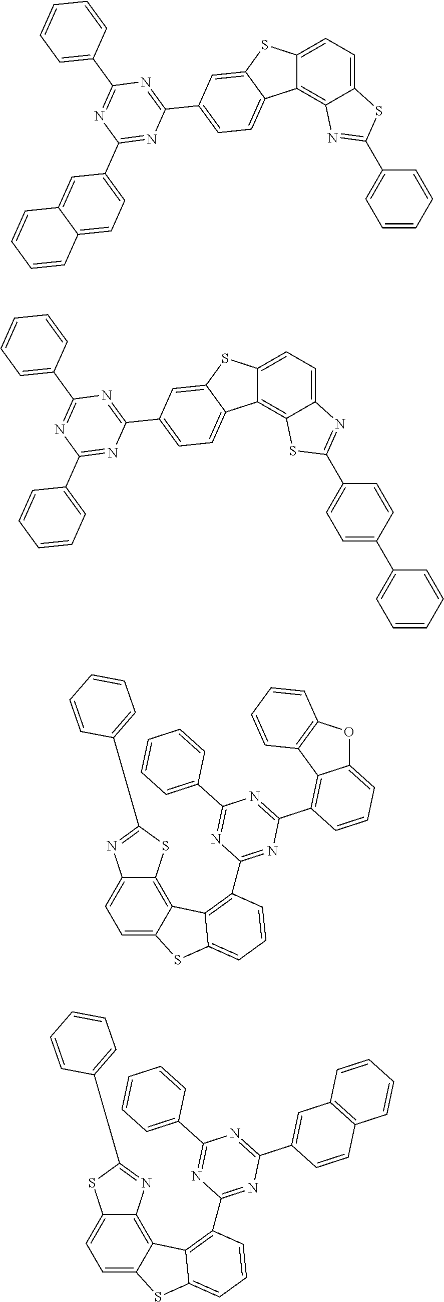 [SMILES: c1ccc(-c2nc(-c3ccc4ccccc4c3)nc(-c3ccc4c(c3)sc3ccc5sc(-c6ccccc6)nc5c34)n2)cc1 ; c1ccc(-c2ccc(-c3nc4ccc5sc6cc(-c7nc(-c8ccccc8)nc(-c8ccccc8)n7)ccc6c5c4s3)cc2)cc1 ; c1ccc(-c2nc(-c3cccc4oc5ccccc5c34)nc(-c3cccc4sc5ccc6nc(-c7ccccc7)sc6c5c34)n2)cc1 ; c1ccc(-c2nc(-c3ccc4ccccc4c3)nc(-c3cccc4sc5ccc6sc(-c7ccccc7)nc6c5c34)n2)cc1]

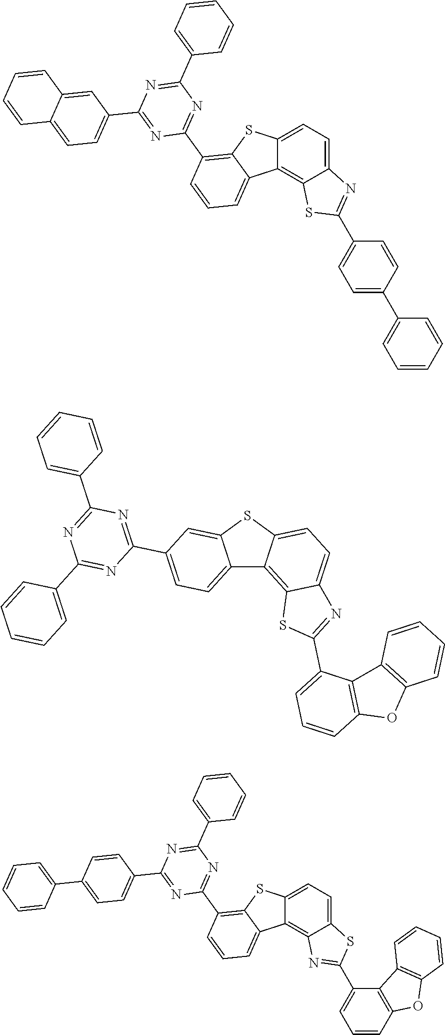 [SMILES: c1ccc(-c2ccc(-c3nc4ccc5sc6c(-c7nc(-c8ccccc8)nc(-c8ccc9ccccc9c8)n7)cccc6c5c4s3)cc2)cc1 ; c1ccc(-c2nc(-c3ccccc3)nc(-c3ccc4c(c3)sc3ccc5nc(-c6cccc7oc8ccccc8c67)sc5c34)n2)cc1 ; c1ccc(-c2ccc(-c3nc(-c4ccccc4)nc(-c4cccc5c4sc4ccc6sc(-c7cccc8oc9ccccc9c78)nc6c45)n3)cc2)cc1]

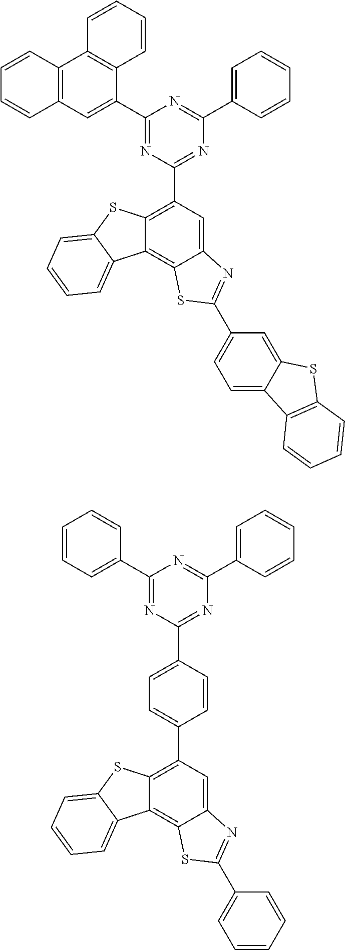 [SMILES: c1ccc(-c2nc(-c3cc4ccccc4c4ccccc34)nc(-c3cc4nc(-c5ccc6c(c5)sc5ccccc56)sc4c4c3sc3ccccc34)n2)cc1 ; c1ccc(-c2nc(-c3ccccc3)nc(-c3ccc(-c4cc5nc(-c6ccccc6)sc5c5c4sc4ccccc45)cc3)n2)cc1]

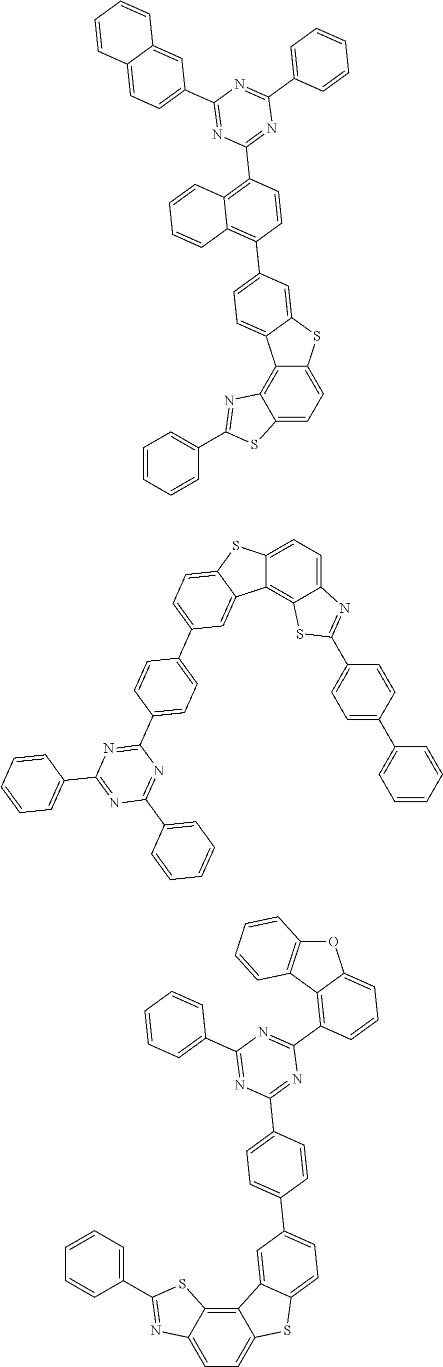 [SMILES: c1ccc(-c2nc(-c3ccc4ccccc4c3)nc(-c3ccc(-c4ccc5c(c4)sc4ccc6sc(-c7ccccc7)nc6c45)c4ccccc34)n2)cc1 ; c1ccc(-c2ccc(-c3nc4ccc5sc6ccc(-c7ccc(-c8nc(-c9ccccc9)nc(-c9ccccc9)n8)cc7)cc6c5c4s3)cc2)cc1 ; c1ccc(-c2nc(-c3ccc(-c4ccc5sc6ccc7nc(-c8ccccc8)sc7c6c5c4)cc3)nc(-c3cccc4oc5ccccc5c34)n2)cc1]

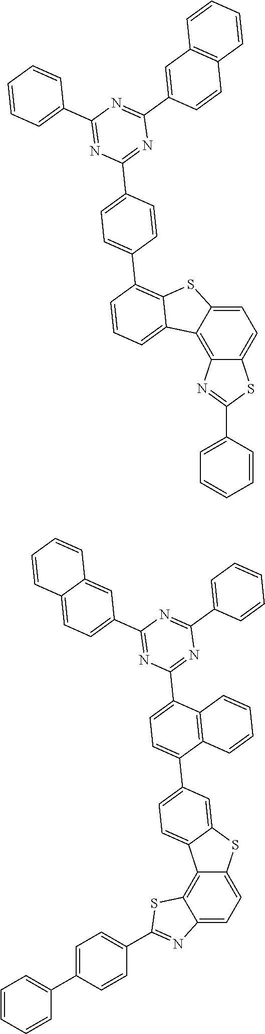 [SMILES: c1ccc(-c2nc(-c3ccc(-c4cccc5c4sc4ccc6sc(-c7ccccc7)nc6c45)cc3)nc(-c3ccc4ccccc4c3)n2)cc1 ; c1ccc(-c2ccc(-c3nc4ccc5sc6cc(-c7ccc(-c8nc(-c9ccccc9)nc(-c9ccc%10ccccc%10c9)n8)c8ccccc78)ccc6c5c4s3)cc2)cc1]

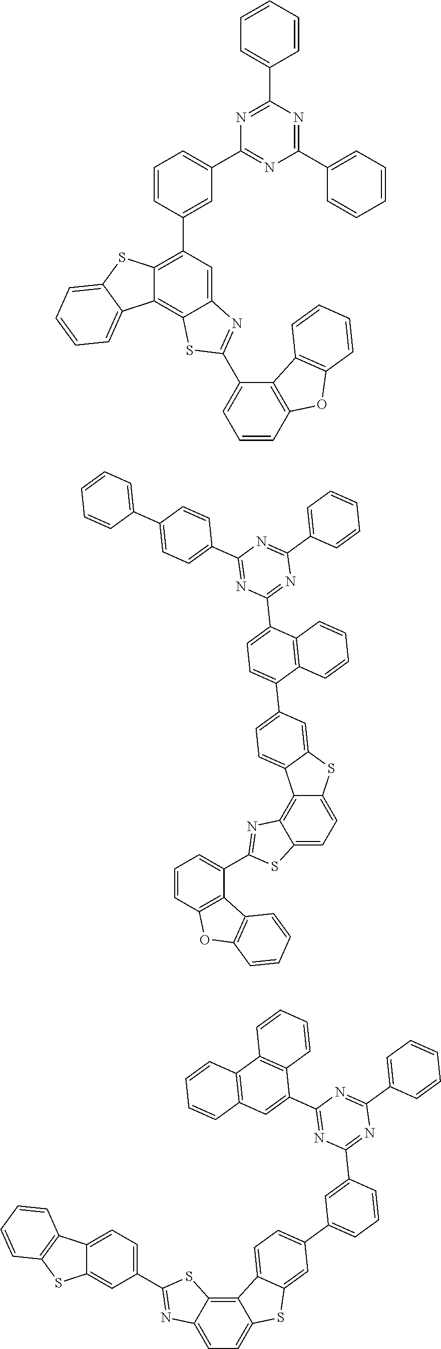 [SMILES: c1ccc(-c2nc(-c3ccccc3)nc(-c3cccc(-c4cc5nc(-c6cccc7oc8ccccc8c67)sc5c5c4sc4ccccc45)c3)n2)cc1 ; c1ccc(-c2ccc(-c3nc(-c4ccccc4)nc(-c4ccc(-c5ccc6c(c5)sc5ccc7sc(-c8cccc9oc%10ccccc%10c89)nc7c56)c5ccccc45)n3)cc2)cc1 ; c1ccc(-c2nc(-c3cccc(-c4ccc5c(c4)sc4ccc6nc(-c7ccc8c(c7)sc7ccccc78)sc6c45)c3)nc(-c3cc4ccccc4c4ccccc34)n2)cc1]

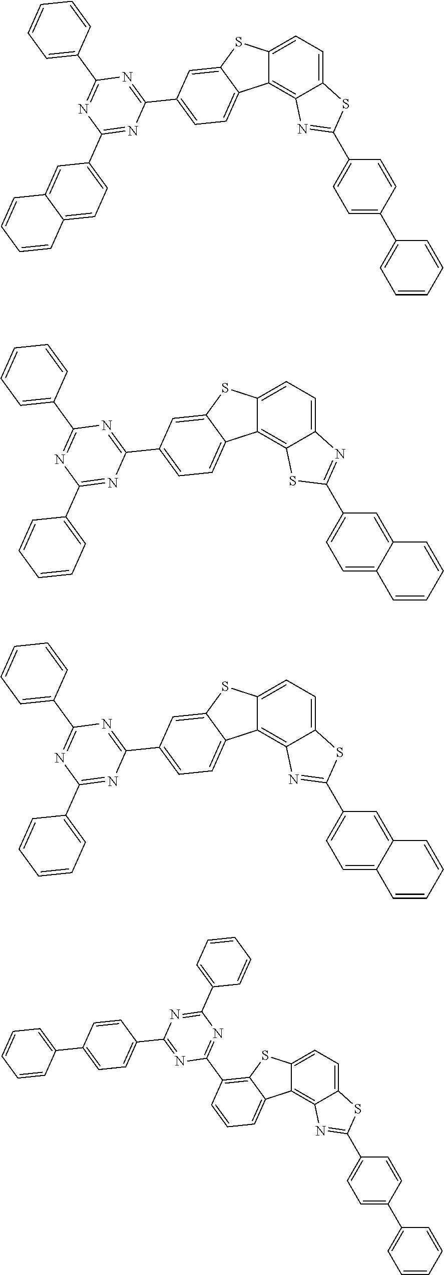 [SMILES: c1ccc(-c2ccc(-c3nc4c(ccc5sc6cc(-c7nc(-c8ccccc8)nc(-c8ccc9ccccc9c8)n7)ccc6c54)s3)cc2)cc1 ; c1ccc(-c2nc(-c3ccccc3)nc(-c3ccc4c(c3)sc3ccc5nc(-c6ccc7ccccc7c6)sc5c34)n2)cc1 ; c1ccc(-c2nc(-c3ccccc3)nc(-c3ccc4c(c3)sc3ccc5sc(-c6ccc7ccccc7c6)nc5c34)n2)cc1 ; c1ccc(-c2ccc(-c3nc(-c4ccccc4)nc(-c4cccc5c4sc4ccc6sc(-c7ccc(-c8ccccc8)cc7)nc6c45)n3)cc2)cc1]

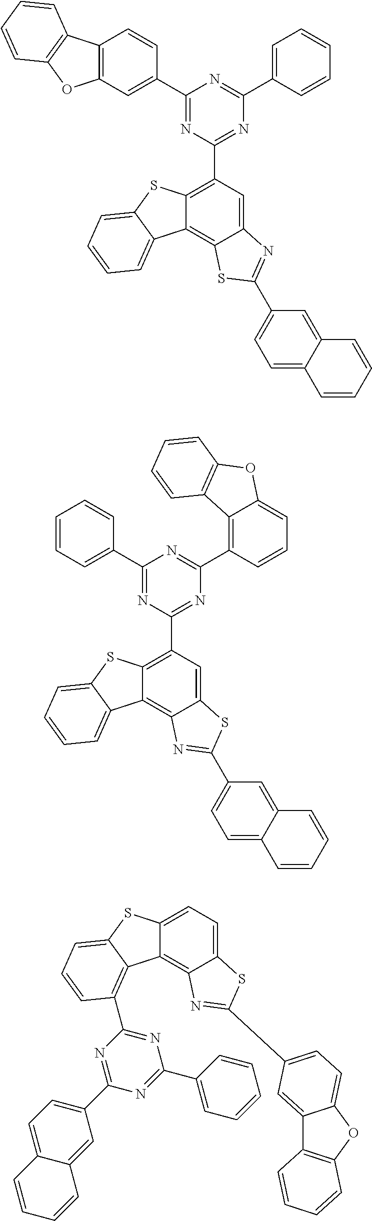 [SMILES: c1ccc(-c2nc(-c3ccc4c(c3)oc3ccccc34)nc(-c3cc4nc(-c5ccc6ccccc6c5)sc4c4c3sc3ccccc34)n2)cc1 ; c1ccc(-c2nc(-c3cc4sc(-c5ccc6ccccc6c5)nc4c4c3sc3ccccc34)nc(-c3cccc4oc5ccccc5c34)n2)cc1 ; c1ccc(-c2nc(-c3ccc4ccccc4c3)nc(-c3cccc4sc5ccc6sc(-c7ccc8oc9ccccc9c8c7)nc6c5c34)n2)cc1]

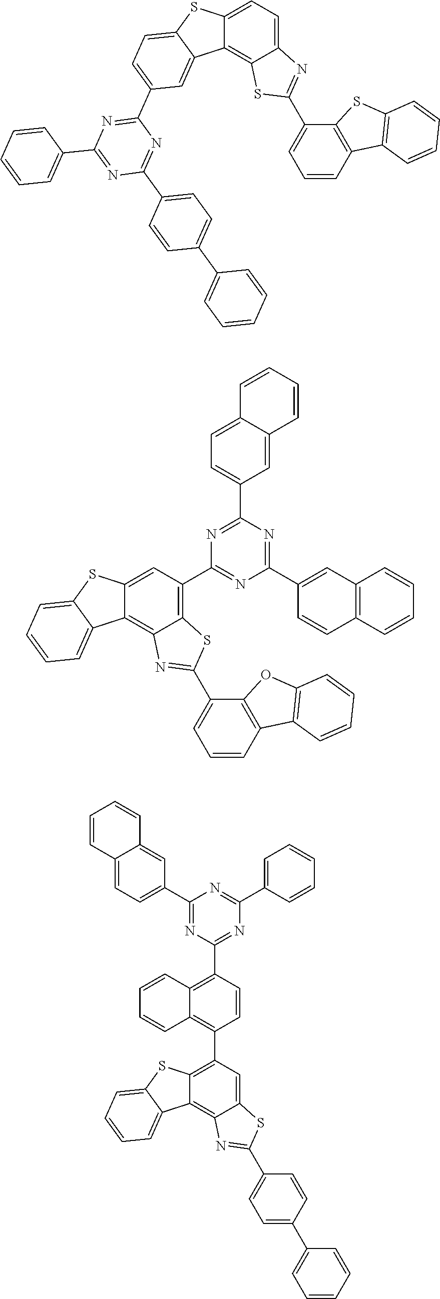 [SMILES: c1ccc(-c2ccc(-c3nc(-c4ccccc4)nc(-c4ccc5sc6ccc7nc(-c8cccc9c8sc8ccccc89)sc7c6c5c4)n3)cc2)cc1 ; c1ccc2cc(-c3nc(-c4ccc5ccccc5c4)nc(-c4cc5sc6ccccc6c5c5nc(-c6cccc7c6oc6ccccc67)sc45)n3)ccc2c1 ; c1ccc(-c2ccc(-c3nc4c(cc(-c5ccc(-c6nc(-c7ccccc7)nc(-c7ccc8ccccc8c7)n6)c6ccccc56)c5sc6ccccc6c54)s3)cc2)cc1]

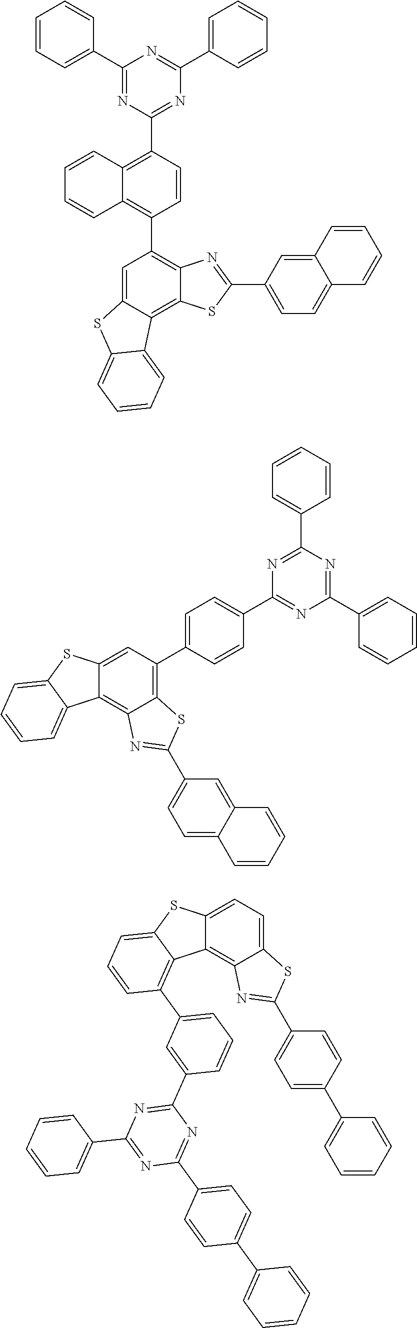

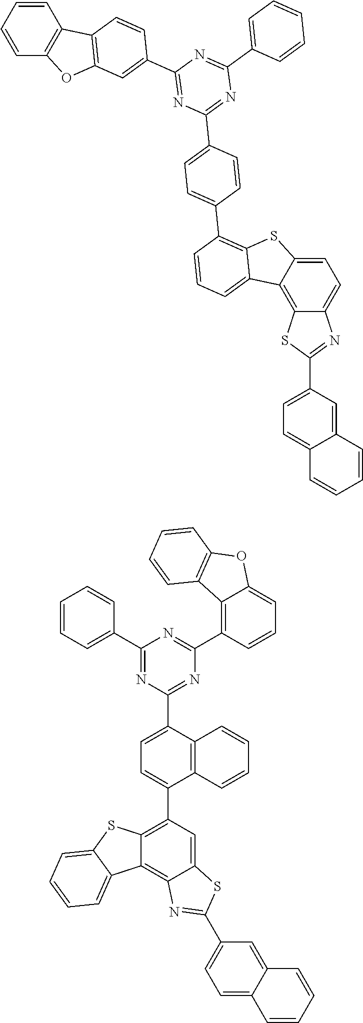 [SMILES: c1ccc(-c2nc(-c3ccc(-c4cccc5c4sc4ccc6nc(-c7ccc8ccccc8c7)sc6c45)cc3)nc(-c3ccc4c(c3)oc3ccccc34)n2)cc1 ; c1ccc(-c2nc(-c3ccc(-c4cc5sc(-c6ccc7ccccc7c6)nc5c5c4sc4ccccc45)c4ccccc34)nc(-c3cccc4oc5ccccc5c34)n2)cc1]

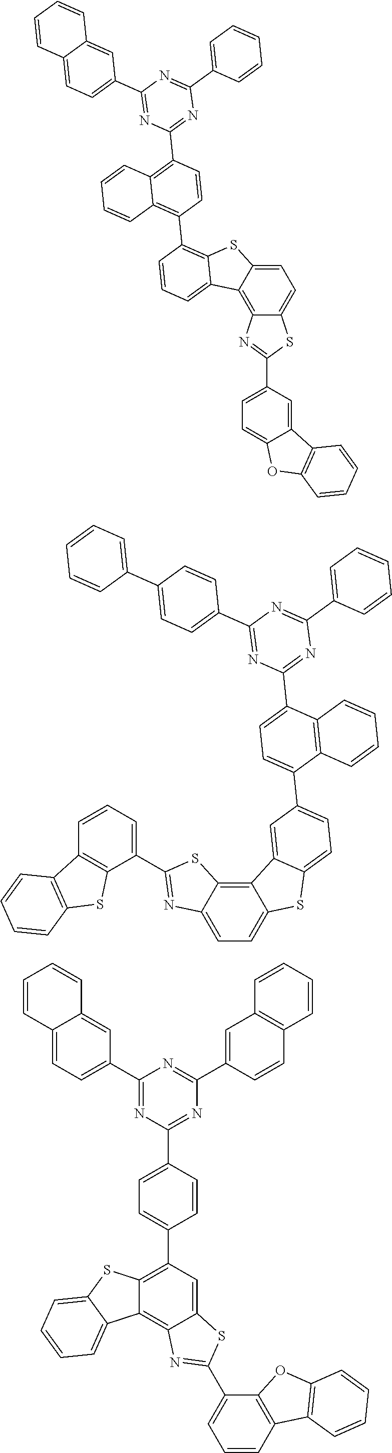

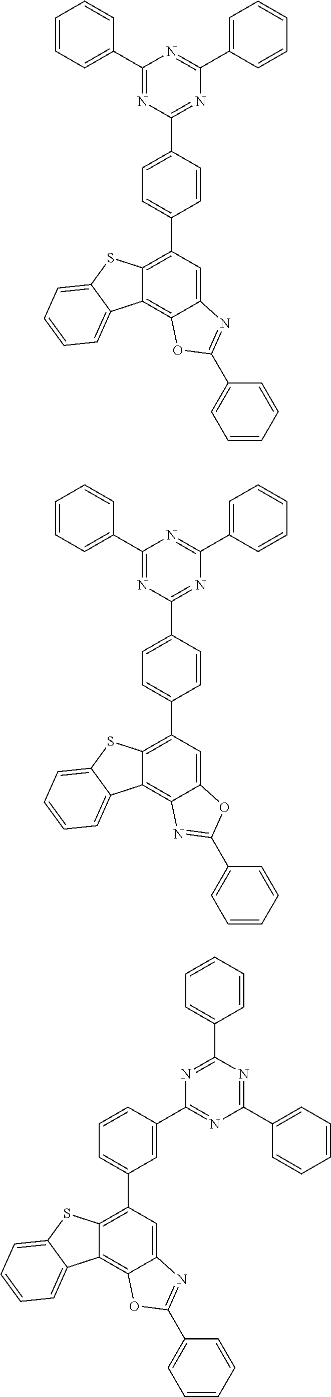

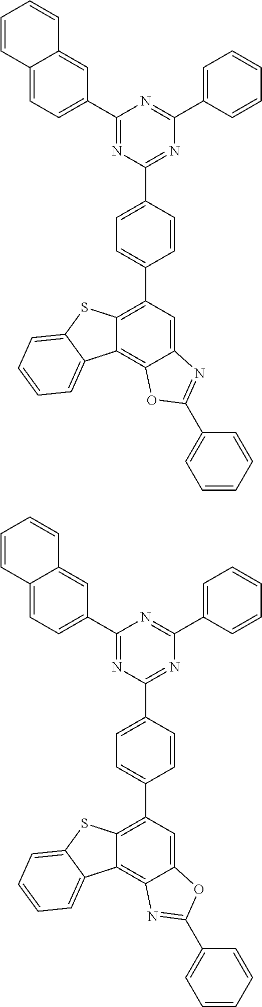 [SMILES: c1ccc(-c2nc(-c3ccc(-c4cc5nc(-c6ccccc6)oc5c5c4sc4ccccc45)cc3)nc(-c3ccc4ccccc4c3)n2)cc1 ; c1ccc(-c2nc(-c3ccc(-c4cc5oc(-c6ccccc6)nc5c5c4sc4ccccc45)cc3)nc(-c3ccc4ccccc4c3)n2)cc1]

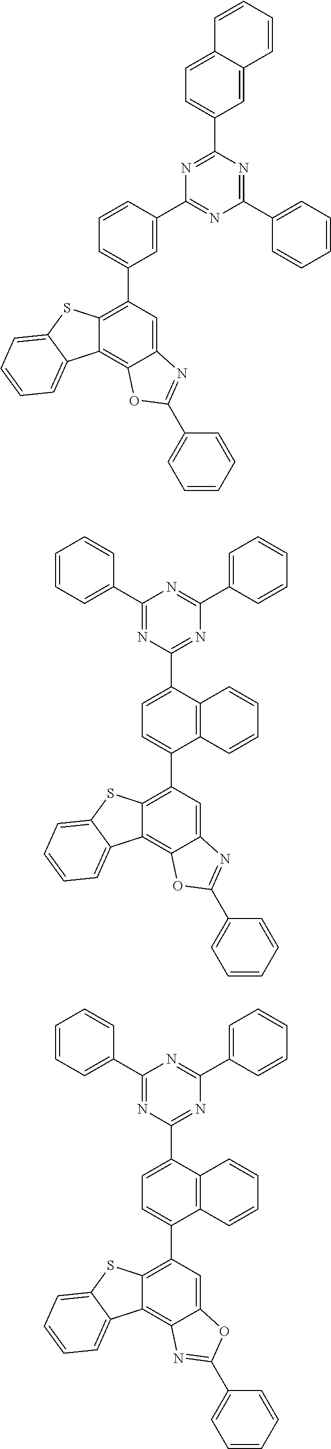

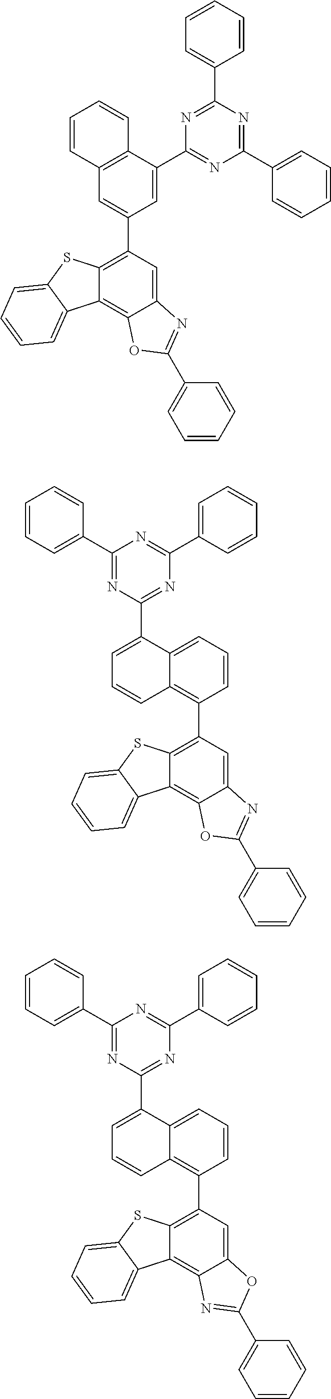

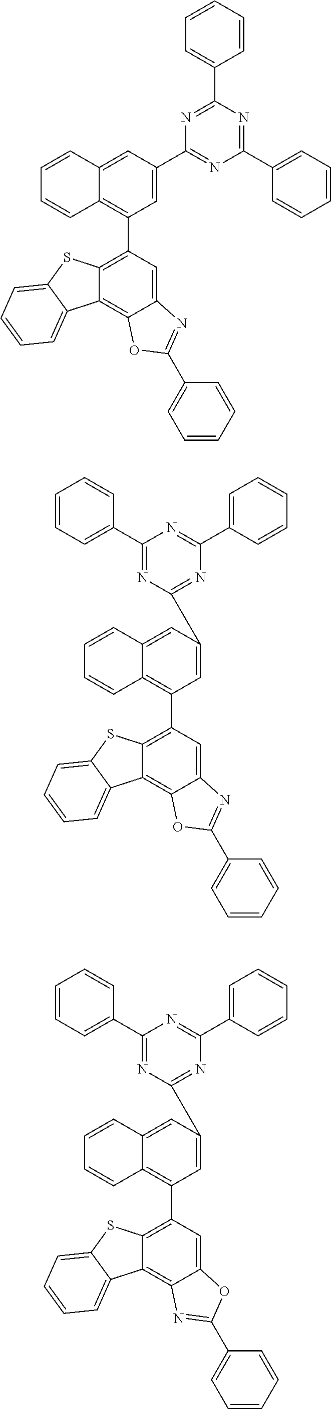 [SMILES: c1ccc(-c2nc(-c3ccccc3)nc(-c3cc(-c4cc5nc(-c6ccccc6)oc5c5c4sc4ccccc45)c4ccccc4c3)n2)cc1 ; c1ccc(-c2nc(-c3ccccc3)nc(-c3cc(-c4cc5nc(-c6ccccc6)oc5c5c4sc4ccccc45)c4ccccc4c3)n2)cc1 ; c1ccc(-c2nc(-c3ccccc3)nc(-c3cc(-c4cc5oc(-c6ccccc6)nc5c5c4sc4ccccc45)c4ccccc4c3)n2)cc1]

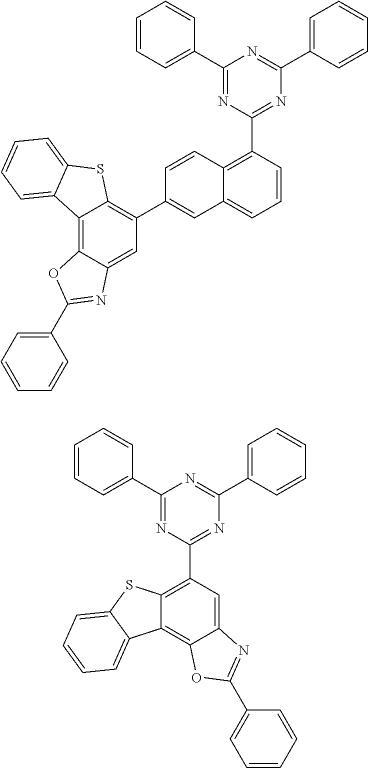 [SMILES: c1ccc(-c2nc(-c3ccccc3)nc(-c3cccc4cc(-c5cc6nc(-c7ccccc7)oc6c6c5sc5ccccc56)ccc34)n2)cc1 ; c1ccc(-c2nc(-c3ccccc3)nc(-c3cc4nc(-c5ccccc5)oc4c4c3sc3ccccc34)n2)cc1]

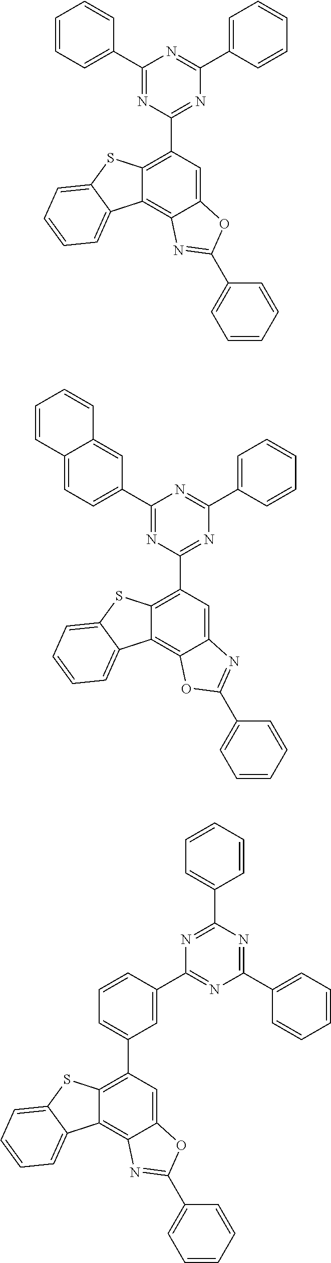 [SMILES: c1ccc(-c2nc(-c3ccccc3)nc(-c3cc4oc(-c5ccccc5)nc4c4c3sc3ccccc34)n2)cc1 ; c1ccc(-c2nc(-c3ccc4ccccc4c3)nc(-c3cc4nc(-c5ccccc5)oc4c4c3sc3ccccc34)n2)cc1 ; c1ccc(-c2nc(-c3ccccc3)nc(-c3cccc(-c4cc5oc(-c6ccccc6)nc5c5c4sc4ccccc45)c3)n2)cc1]

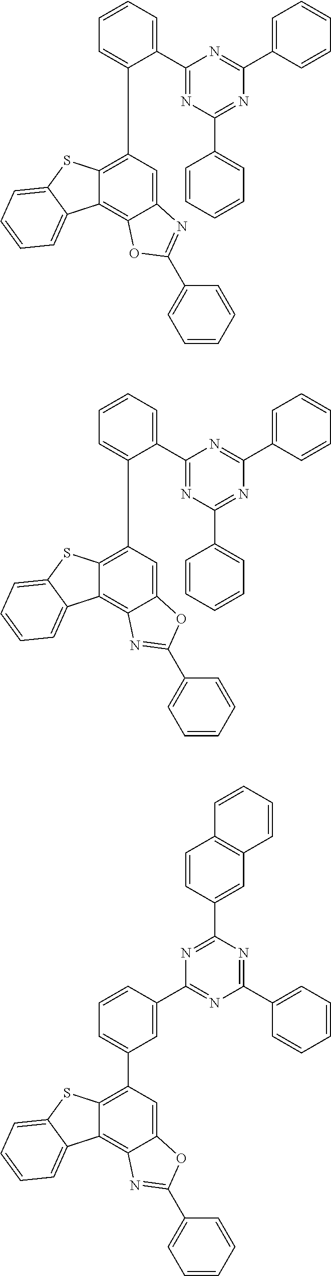 [SMILES: c1ccc(-c2nc(-c3ccccc3)nc(-c3ccccc3-c3cc4nc(-c5ccccc5)oc4c4c3sc3ccccc34)n2)cc1 ; c1ccc(-c2nc(-c3ccccc3)nc(-c3ccccc3-c3cc4oc(-c5ccccc5)nc4c4c3sc3ccccc34)n2)cc1 ; c1ccc(-c2nc(-c3cccc(-c4cc5oc(-c6ccccc6)nc5c5c4sc4ccccc45)c3)nc(-c3ccc4ccccc4c3)n2)cc1]

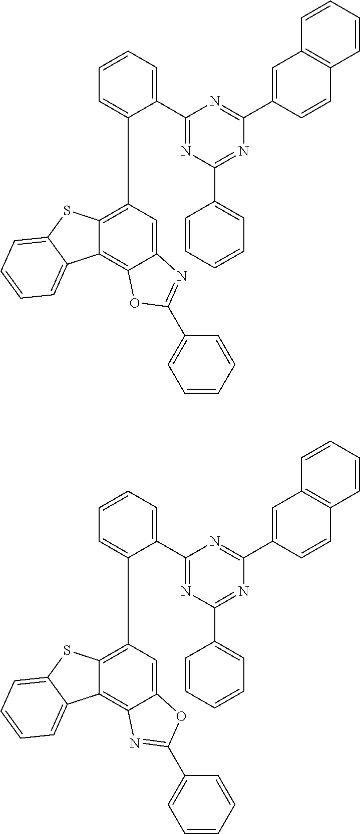 [SMILES: c1ccc(-c2nc(-c3ccc4ccccc4c3)nc(-c3ccccc3-c3cc4nc(-c5ccccc5)oc4c4c3sc3ccccc34)n2)cc1 ; c1ccc(-c2nc(-c3ccc4ccccc4c3)nc(-c3ccccc3-c3cc4oc(-c5ccccc5)nc4c4c3sc3ccccc34)n2)cc1]

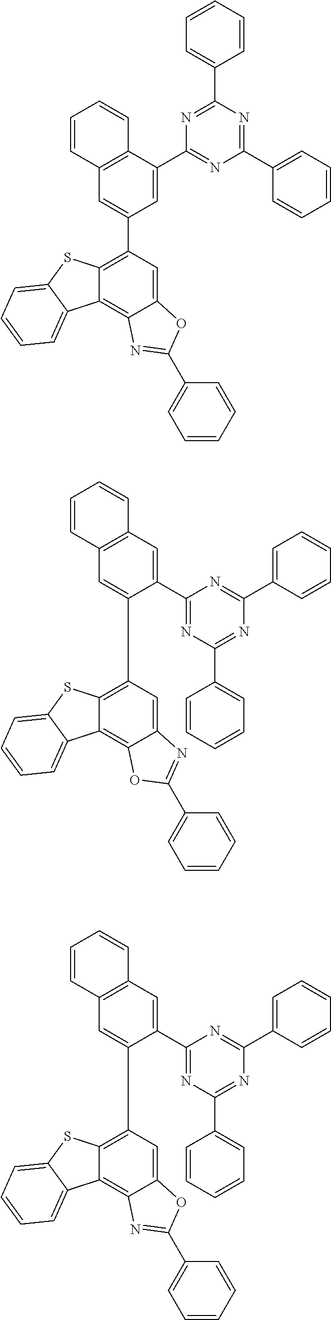 [SMILES: c1ccc(-c2nc(-c3ccccc3)nc(-c3cc(-c4cc5oc(-c6ccccc6)nc5c5c4sc4ccccc45)cc4ccccc34)n2)cc1 ; c1ccc(-c2nc(-c3ccccc3)nc(-c3cc4ccccc4cc3-c3cc4nc(-c5ccccc5)oc4c4c3sc3ccccc34)n2)cc1 ; c1ccc(-c2nc(-c3ccccc3)nc(-c3cc4ccccc4cc3-c3cc4oc(-c5ccccc5)nc4c4c3sc3ccccc34)n2)cc1]

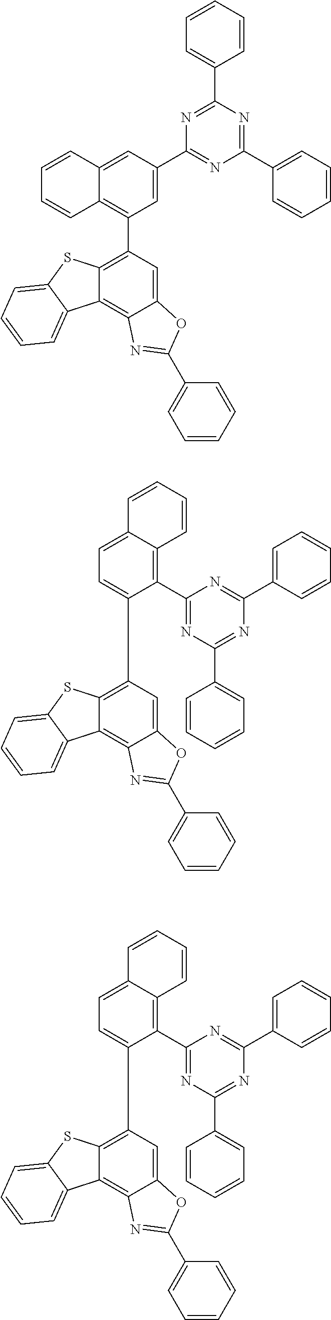 [SMILES: c1ccc(-c2nc(-c3ccccc3)nc(-c3cc(-c4cc5oc(-c6ccccc6)nc5c5c4sc4ccccc45)c4ccccc4c3)n2)cc1 ; c1ccc(-c2nc(-c3ccccc3)nc(-c3c(-c4cc5oc(-c6ccccc6)nc5c5c4sc4ccccc45)ccc4ccccc34)n2)cc1 ; c1ccc(-c2nc(-c3ccccc3)nc(-c3c(-c4cc5oc(-c6ccccc6)nc5c5c4sc4ccccc45)ccc4ccccc34)n2)cc1]

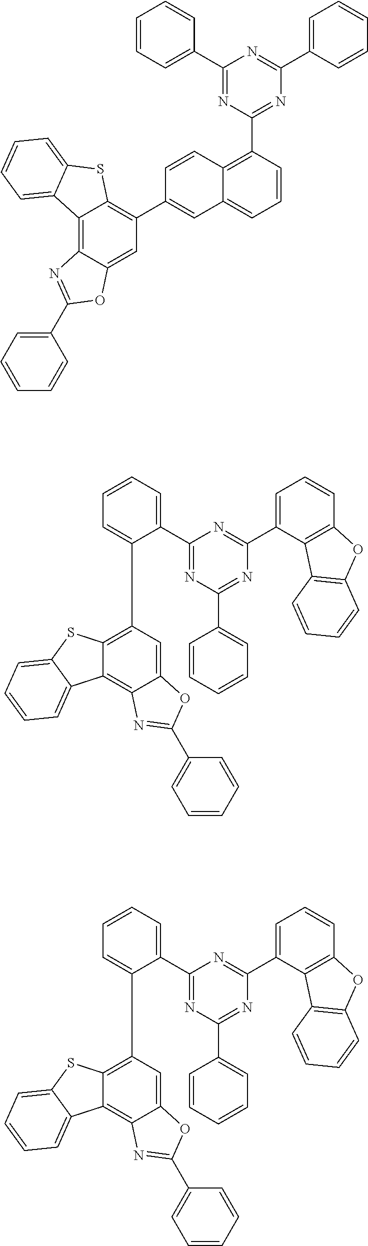 [SMILES: c1ccc(-c2nc(-c3ccccc3)nc(-c3cccc4cc(-c5cc6oc(-c7ccccc7)nc6c6c5sc5ccccc56)ccc34)n2)cc1 ; c1ccc(-c2nc(-c3ccccc3-c3cc4oc(-c5ccccc5)nc4c4c3sc3ccccc34)nc(-c3cccc4oc5ccccc5c34)n2)cc1 ; c1ccc(-c2nc(-c3ccccc3-c3cc4oc(-c5ccccc5)nc4c4c3sc3ccccc34)nc(-c3cccc4oc5ccccc5c34)n2)cc1]

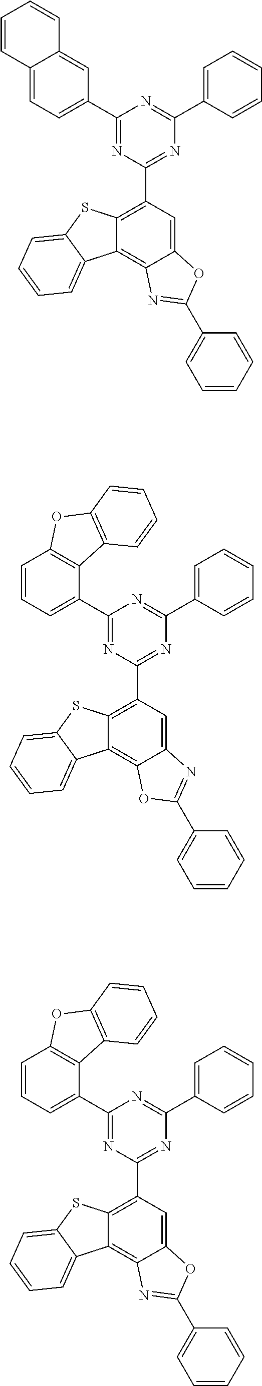 [SMILES: c1ccc(-c2nc(-c3ccc4ccccc4c3)nc(-c3cc4oc(-c5ccccc5)nc4c4c3sc3ccccc34)n2)cc1 ; c1ccc(-c2nc(-c3cc4nc(-c5ccccc5)oc4c4c3sc3ccccc34)nc(-c3cccc4oc5ccccc5c34)n2)cc1 ; c1ccc(-c2nc(-c3cc4oc(-c5ccccc5)nc4c4c3sc3ccccc34)nc(-c3cccc4oc5ccccc5c34)n2)cc1]

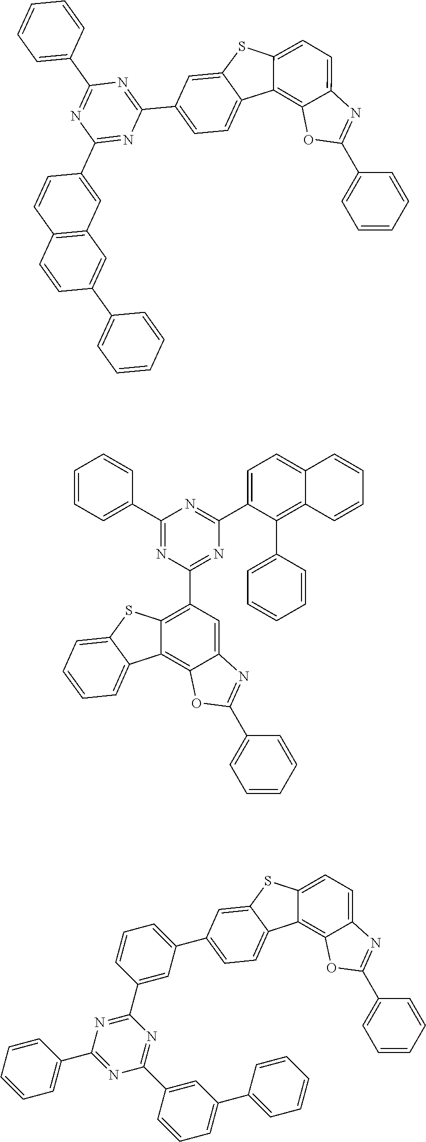 [SMILES: c1ccc(-c2ccc3ccc(-c4nc(-c5ccccc5)nc(-c5ccc6c(c5)sc5ccc7nc(-c8ccccc8)oc7c56)n4)cc3c2)cc1 ; c1ccc(-c2nc(-c3ccc4ccccc4c3-c3ccccc3)nc(-c3cc4nc(-c5ccccc5)oc4c4c3sc3ccccc34)n2)cc1 ; c1ccc(-c2cccc(-c3nc(-c4ccccc4)nc(-c4cccc(-c5ccc6c(c5)sc5ccc7nc(-c8ccccc8)oc7c56)c4)n3)c2)cc1]

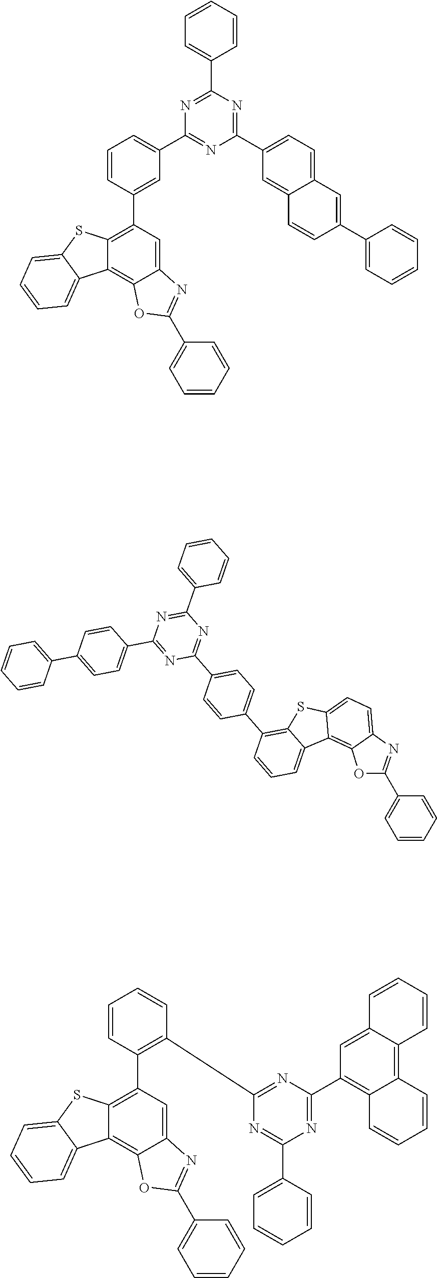 [SMILES: c1ccc(-c2ccc3cc(-c4nc(-c5ccccc5)nc(-c5cccc(-c6cc7nc(-c8ccccc8)oc7c7c6sc6ccccc67)c5)n4)ccc3c2)cc1 ; c1ccc(-c2ccc(-c3nc(-c4ccccc4)nc(-c4ccc(-c5cccc6c5sc5ccc7nc(-c8ccccc8)oc7c56)cc4)n3)cc2)cc1 ; c1ccc(-c2nc(-c3ccccc3-c3cc4nc(-c5ccccc5)oc4c4c3sc3ccccc34)nc(-c3cc4ccccc4c4ccccc34)n2)cc1]

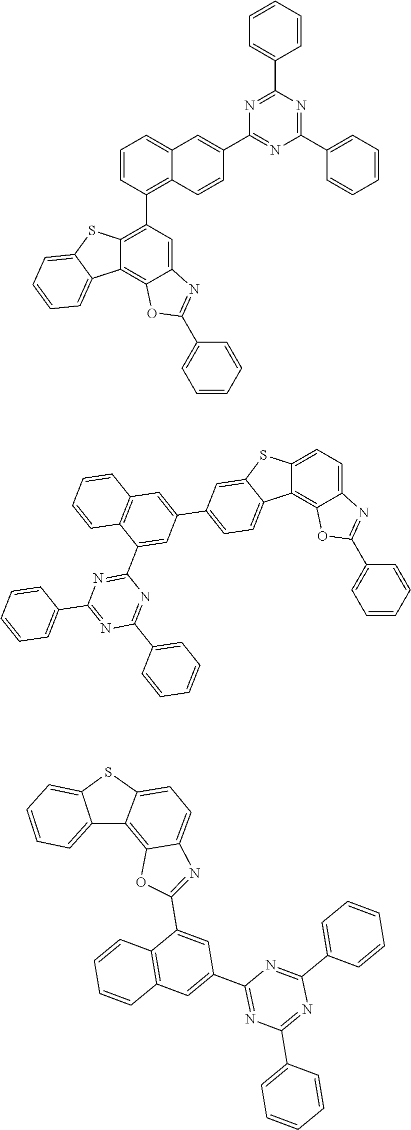 [SMILES: c1ccc(-c2nc(-c3ccccc3)nc(-c3ccc4c(-c5cc6nc(-c7ccccc7)oc6c6c5sc5ccccc56)cccc4c3)n2)cc1 ; c1ccc(-c2nc(-c3ccccc3)nc(-c3cc(-c4ccc5c(c4)sc4ccc6nc(-c7ccccc7)oc6c45)cc4ccccc34)n2)cc1 ; c1ccc(-c2nc(-c3ccccc3)nc(-c3cc(-c4nc5ccc6sc7ccccc7c6c5o4)c4ccccc4c3)n2)cc1]

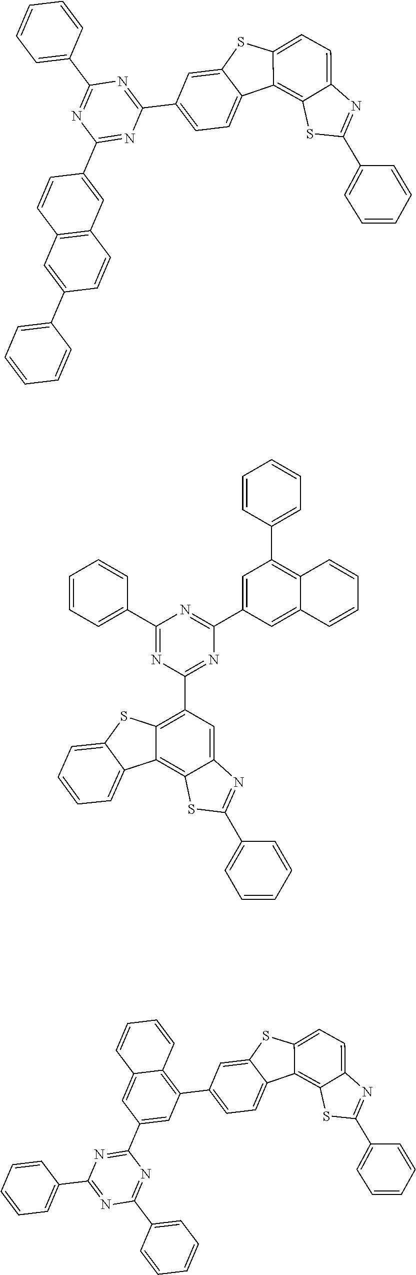 [SMILES: c1ccc(-c2ccc3cc(-c4nc(-c5ccccc5)nc(-c5ccc6c(c5)sc5ccc7nc(-c8ccccc8)sc7c56)n4)ccc3c2)cc1 ; c1ccc(-c2nc(-c3cc(-c4ccccc4)c4ccccc4c3)nc(-c3cc4nc(-c5ccccc5)sc4c4c3sc3ccccc34)n2)cc1 ; c1ccc(-c2nc(-c3ccccc3)nc(-c3cc(-c4ccc5c(c4)sc4ccc6nc(-c7ccccc7)sc6c45)c4ccccc4c3)n2)cc1]

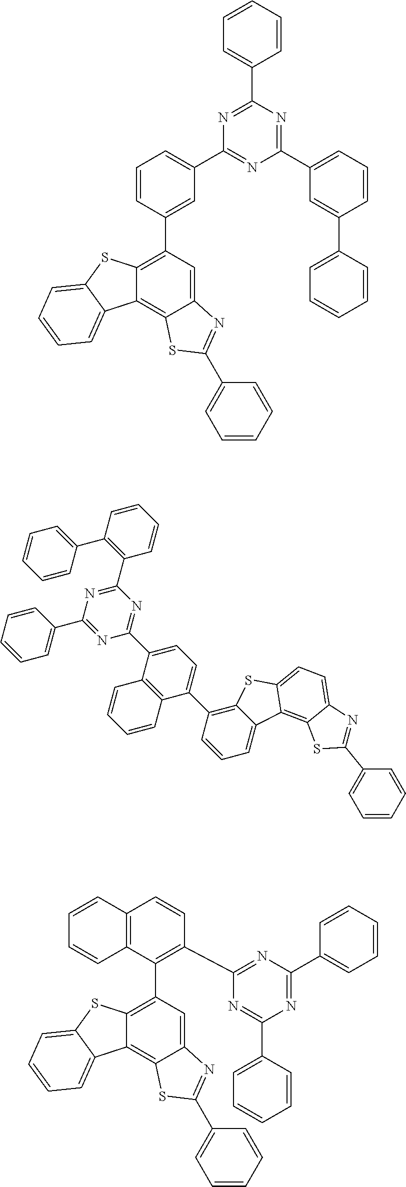 [SMILES: c1ccc(-c2cccc(-c3nc(-c4ccccc4)nc(-c4cccc(-c5cc6nc(-c7ccccc7)sc6c6c5sc5ccccc56)c4)n3)c2)cc1 ; c1ccc(-c2nc(-c3ccccc3-c3ccccc3)nc(-c3ccc(-c4cccc5c4sc4ccc6nc(-c7ccccc7)sc6c45)c4ccccc34)n2)cc1 ; c1ccc(-c2nc(-c3ccccc3)nc(-c3ccc4ccccc4c3-c3cc4nc(-c5ccccc5)sc4c4c3sc3ccccc34)n2)cc1]

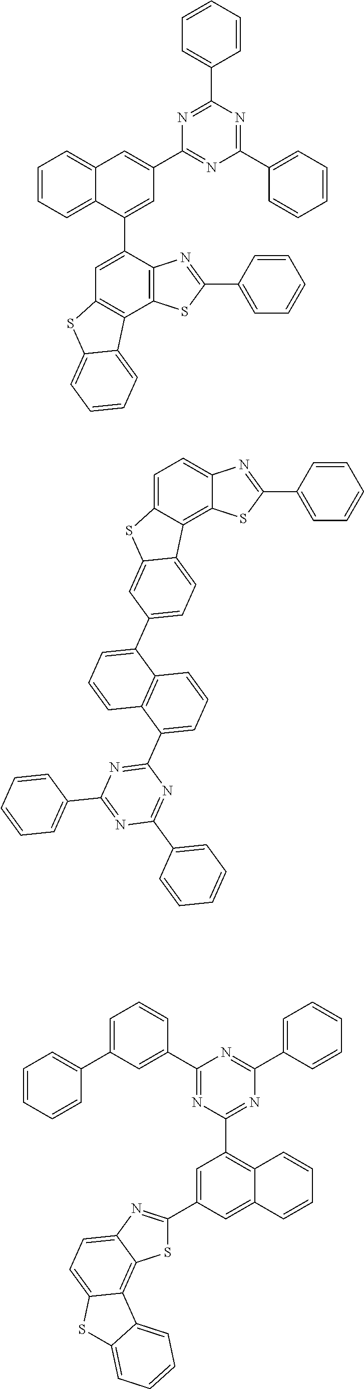 [SMILES: c1ccc(-c2nc(-c3ccccc3)nc(-c3cc(-c4cc5sc6ccccc6c5c5sc(-c6ccccc6)nc45)c4ccccc4c3)n2)cc1 ; c1ccc(-c2nc(-c3ccccc3)nc(-c3cccc4c(-c5ccc6c(c5)sc5ccc7nc(-c8ccccc8)sc7c56)cccc34)n2)cc1 ; c1ccc(-c2cccc(-c3nc(-c4ccccc4)nc(-c4cc(-c5nc6ccc7sc8ccccc8c7c6s5)cc5ccccc45)n3)c2)cc1]

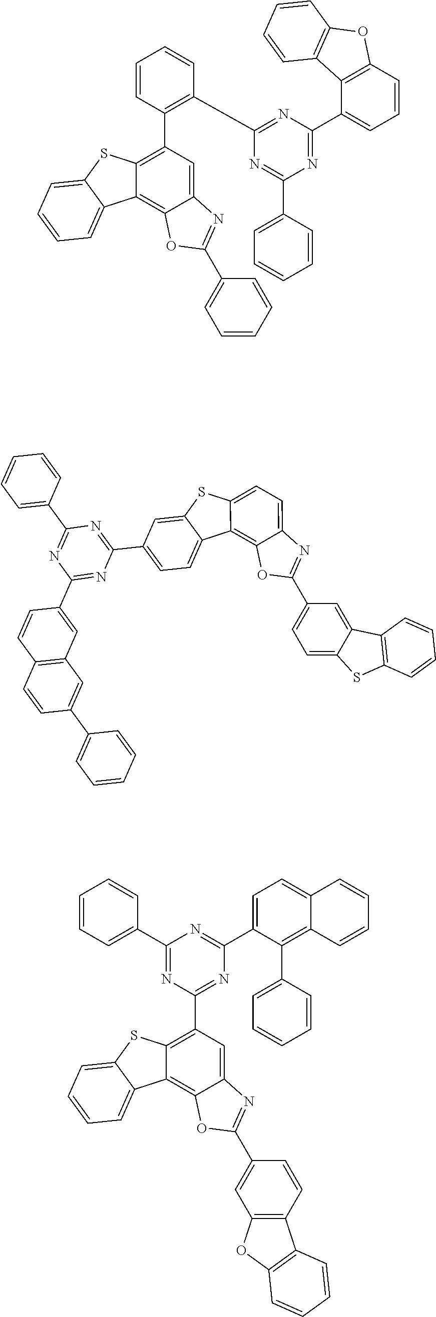 [SMILES: c1ccc(-c2nc(-c3ccccc3-c3cc4nc(-c5ccccc5)oc4c4c3sc3ccccc34)nc(-c3cccc4oc5ccccc5c34)n2)cc1 ; c1ccc(-c2ccc3ccc(-c4nc(-c5ccccc5)nc(-c5ccc6c(c5)sc5ccc7nc(-c8ccc9sc%10ccccc%10c9c8)oc7c56)n4)cc3c2)cc1 ; c1ccc(-c2nc(-c3ccc4ccccc4c3-c3ccccc3)nc(-c3cc4nc(-c5ccc6c(c5)oc5ccccc56)oc4c4c3sc3ccccc34)n2)cc1]

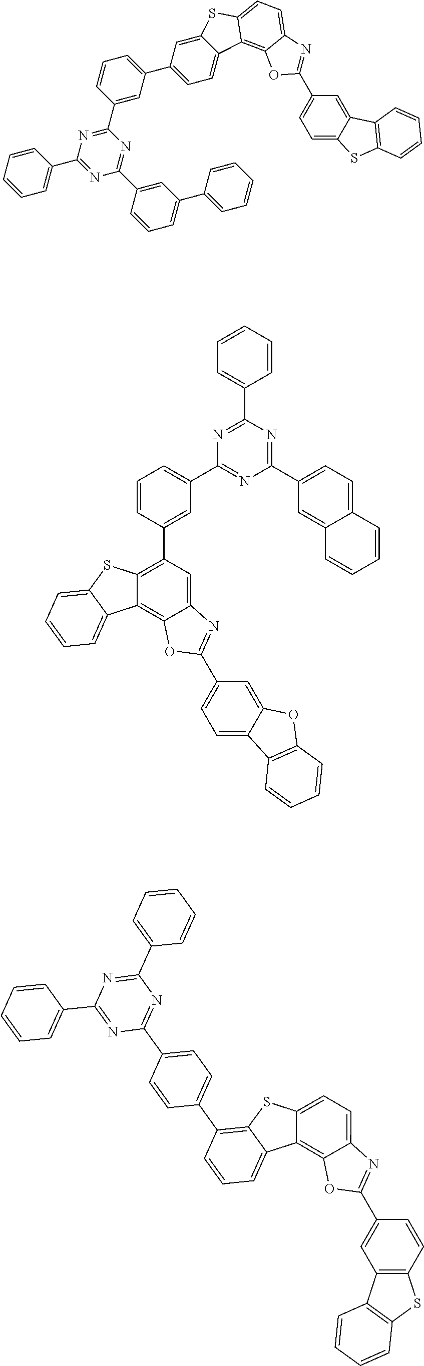 [SMILES: c1ccc(-c2cccc(-c3nc(-c4ccccc4)nc(-c4cccc(-c5ccc6c(c5)sc5ccc7nc(-c8ccc9sc%10ccccc%10c9c8)oc7c56)c4)n3)c2)cc1 ; c1ccc(-c2nc(-c3cccc(-c4cc5nc(-c6ccc7c(c6)oc6ccccc67)oc5c5c4sc4ccccc45)c3)nc(-c3ccc4ccccc4c3)n2)cc1 ; c1ccc(-c2nc(-c3ccccc3)nc(-c3ccc(-c4cccc5c4sc4ccc6nc(-c7ccc8sc9ccccc9c8c7)oc6c45)cc3)n2)cc1]

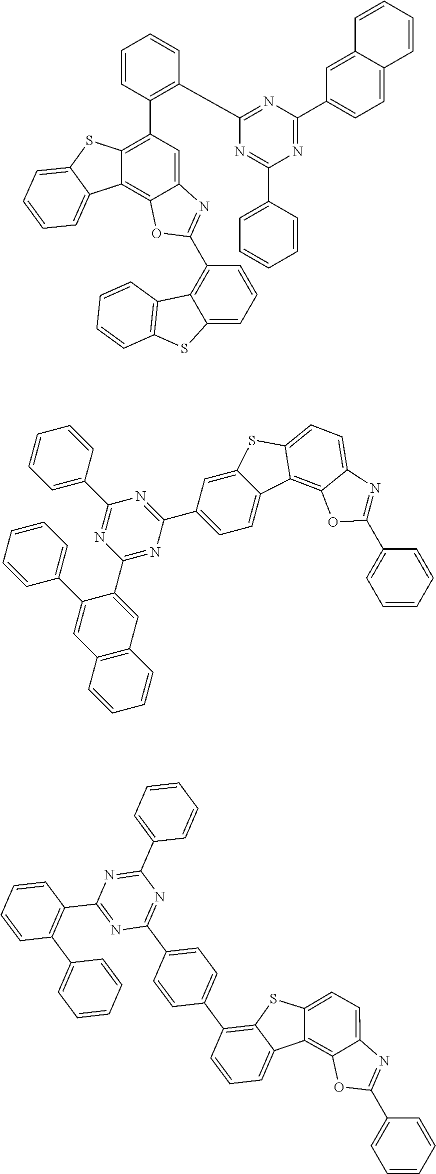 [SMILES: c1ccc(-c2nc(-c3ccc4ccccc4c3)nc(-c3ccccc3-c3cc4nc(-c5cccc6sc7ccccc7c56)oc4c4c3sc3ccccc34)n2)cc1 ; c1ccc(-c2nc(-c3ccc4c(c3)sc3ccc5nc(-c6ccccc6)oc5c34)nc(-c3cc4ccccc4cc3-c3ccccc3)n2)cc1 ; c1ccc(-c2nc(-c3ccc(-c4cccc5c4sc4ccc6nc(-c7ccccc7)oc6c45)cc3)nc(-c3ccccc3-c3ccccc3)n2)cc1]

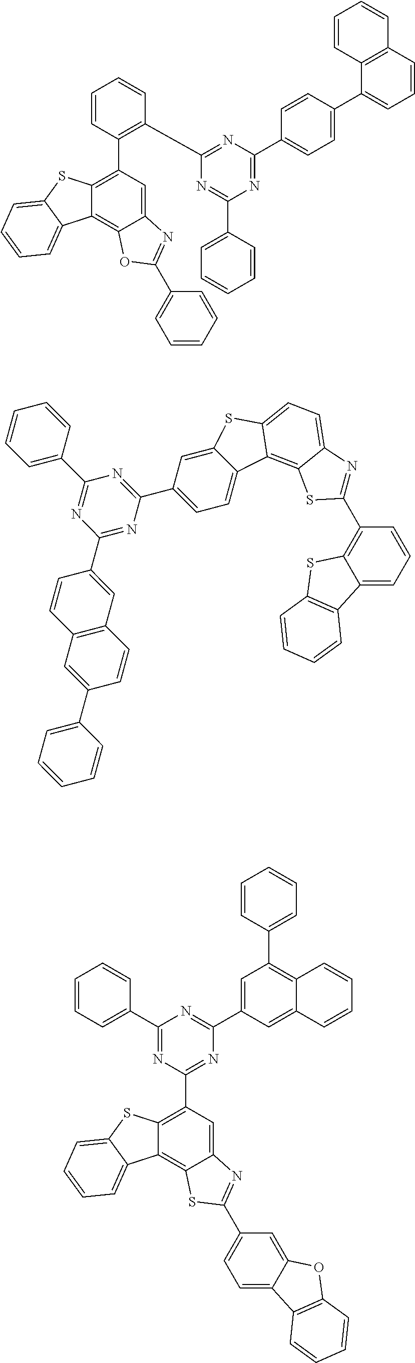 [SMILES: c1ccc(-c2nc(-c3ccc(-c4cccc5ccccc45)cc3)nc(-c3ccccc3-c3cc4nc(-c5ccccc5)oc4c4c3sc3ccccc34)n2)cc1 ; c1ccc(-c2ccc3cc(-c4nc(-c5ccccc5)nc(-c5ccc6c(c5)sc5ccc7nc(-c8cccc9c8sc8ccccc89)sc7c56)n4)ccc3c2)cc1 ; c1ccc(-c2nc(-c3cc(-c4ccccc4)c4ccccc4c3)nc(-c3cc4nc(-c5ccc6c(c5)oc5ccccc56)sc4c4c3sc3ccccc34)n2)cc1]

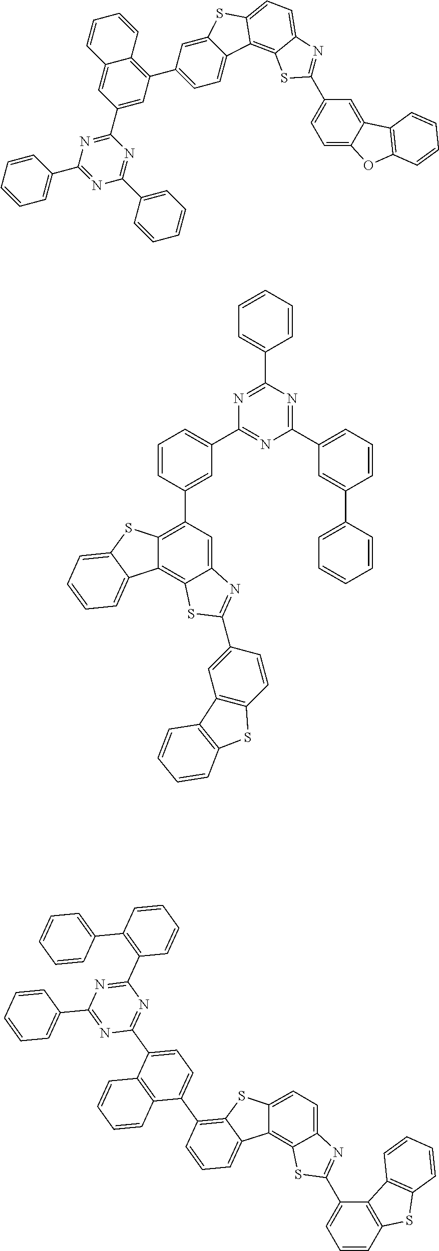 [SMILES: c1ccc(-c2nc(-c3ccccc3)nc(-c3cc(-c4ccc5c(c4)sc4ccc6nc(-c7ccc8oc9ccccc9c8c7)sc6c45)c4ccccc4c3)n2)cc1 ; c1ccc(-c2cccc(-c3nc(-c4ccccc4)nc(-c4cccc(-c5cc6nc(-c7ccc8sc9ccccc9c8c7)sc6c6c5sc5ccccc56)c4)n3)c2)cc1 ; c1ccc(-c2nc(-c3ccccc3-c3ccccc3)nc(-c3ccc(-c4cccc5c4sc4ccc6nc(-c7cccc8sc9ccccc9c78)sc6c45)c4ccccc34)n2)cc1]

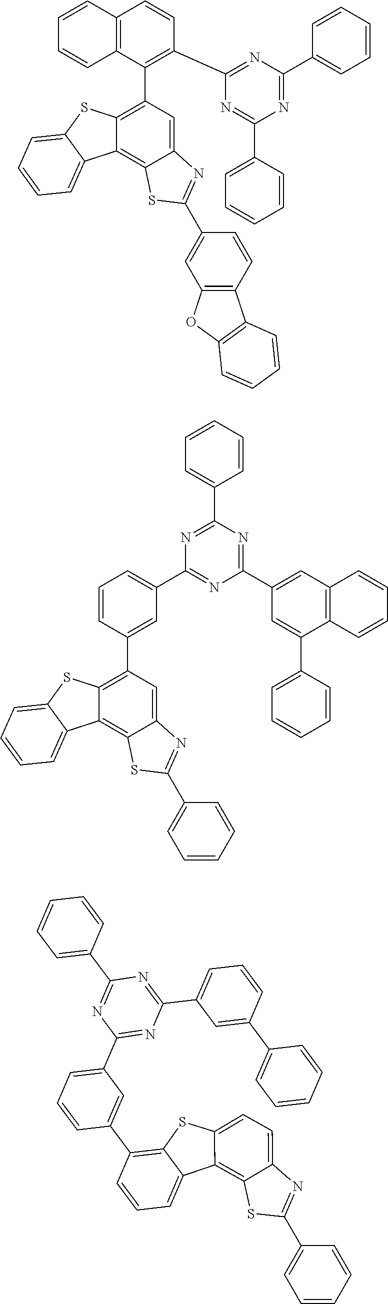

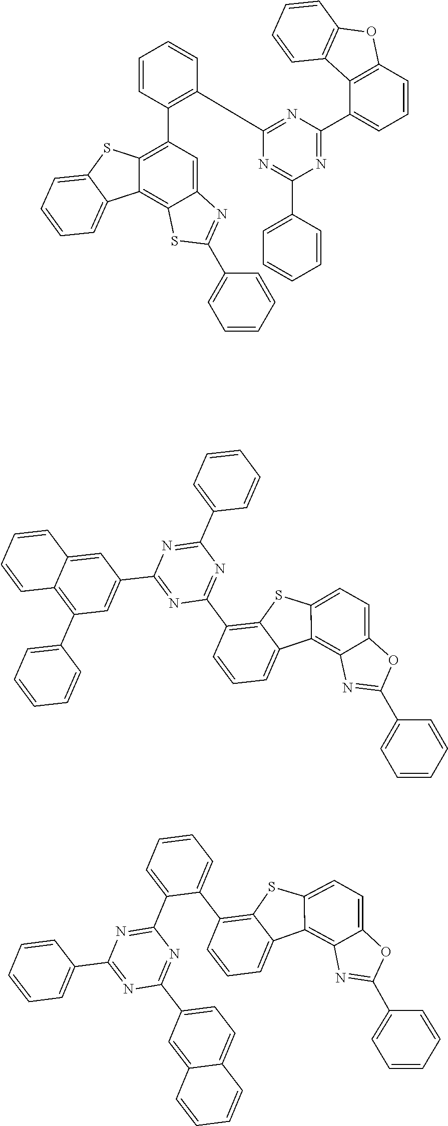 [SMILES: c1ccc(-c2nc(-c3ccccc3-c3cc4nc(-c5ccccc5)sc4c4c3sc3ccccc34)nc(-c3cccc4oc5ccccc5c34)n2)cc1 ; c1ccc(-c2nc(-c3cc(-c4ccccc4)c4ccccc4c3)nc(-c3cccc4c3sc3ccc5oc(-c6ccccc6)nc5c34)n2)cc1 ; c1ccc(-c2nc(-c3ccc4ccccc4c3)nc(-c3ccccc3-c3cccc4c3sc3ccc5oc(-c6ccccc6)nc5c34)n2)cc1]

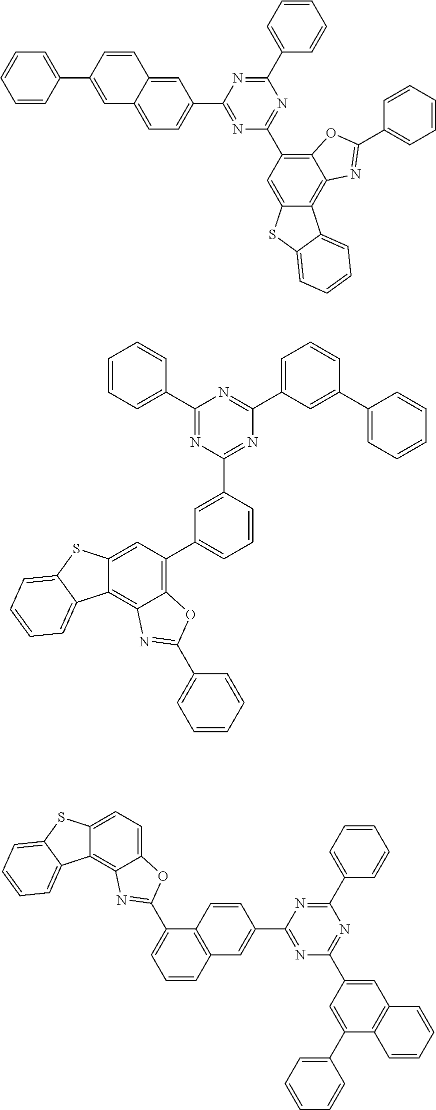 [SMILES: c1ccc(-c2ccc3cc(-c4nc(-c5ccccc5)nc(-c5cc6sc7ccccc7c6c6nc(-c7ccccc7)oc56)n4)ccc3c2)cc1 ; c1ccc(-c2cccc(-c3nc(-c4ccccc4)nc(-c4cccc(-c5cc6sc7ccccc7c6c6nc(-c7ccccc7)oc56)c4)n3)c2)cc1 ; c1ccc(-c2nc(-c3cc(-c4ccccc4)c4ccccc4c3)nc(-c3ccc4c(-c5nc6c(ccc7sc8ccccc8c76)o5)cccc4c3)n2)cc1]

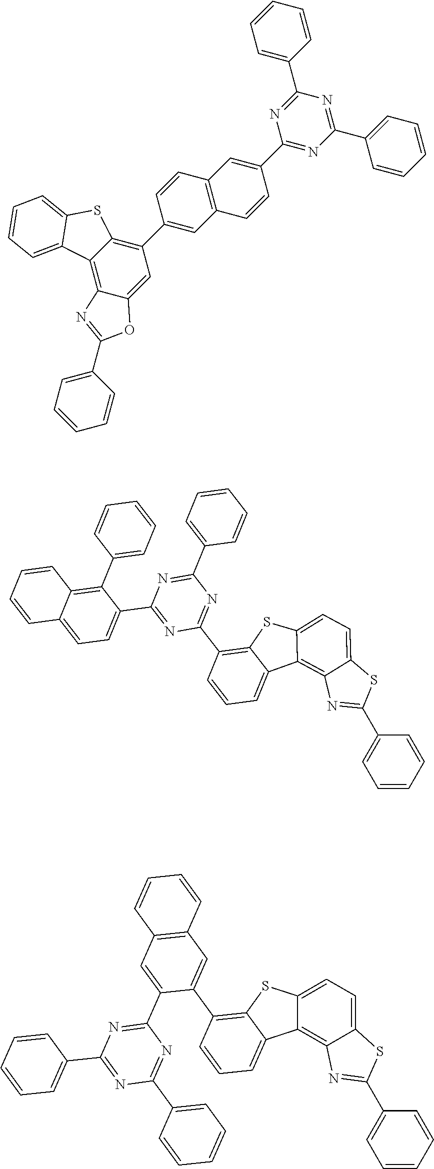 [SMILES: c1ccc(-c2nc(-c3ccccc3)nc(-c3ccc4cc(-c5cc6oc(-c7ccccc7)nc6c6c5sc5ccccc56)ccc4c3)n2)cc1 ; c1ccc(-c2nc(-c3ccc4ccccc4c3-c3ccccc3)nc(-c3cccc4c3sc3ccc5sc(-c6ccccc6)nc5c34)n2)cc1 ; c1ccc(-c2nc(-c3ccccc3)nc(-c3cc4ccccc4cc3-c3cccc4c3sc3ccc5sc(-c6ccccc6)nc5c34)n2)cc1]

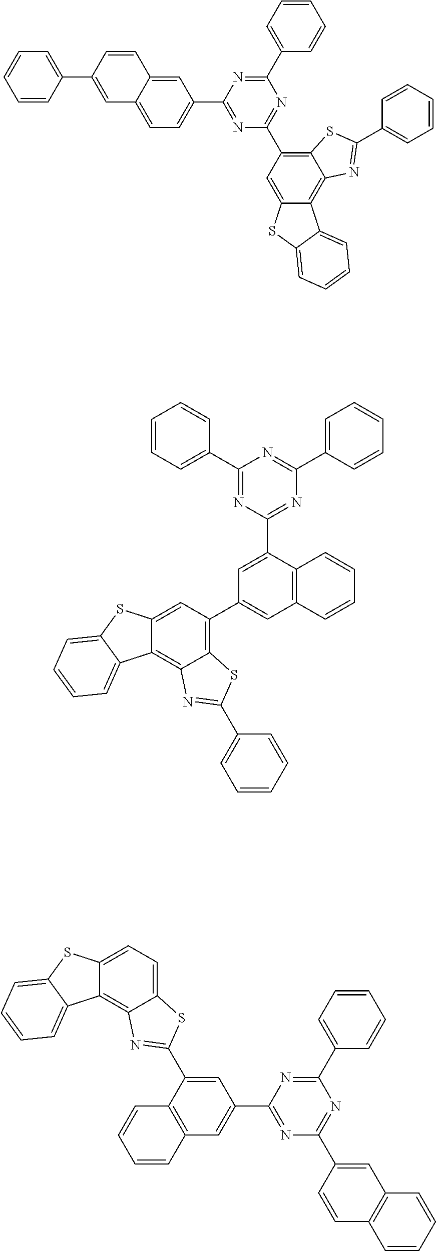 [SMILES: c1ccc(-c2ccc3cc(-c4nc(-c5ccccc5)nc(-c5cc6sc7ccccc7c6c6nc(-c7ccccc7)sc56)n4)ccc3c2)cc1 ; c1ccc(-c2nc(-c3ccccc3)nc(-c3cc(-c4cc5sc6ccccc6c5c5nc(-c6ccccc6)sc45)cc4ccccc34)n2)cc1 ; c1ccc(-c2nc(-c3ccc4ccccc4c3)nc(-c3cc(-c4nc5c(ccc6sc7ccccc7c65)s4)c4ccccc4c3)n2)cc1]

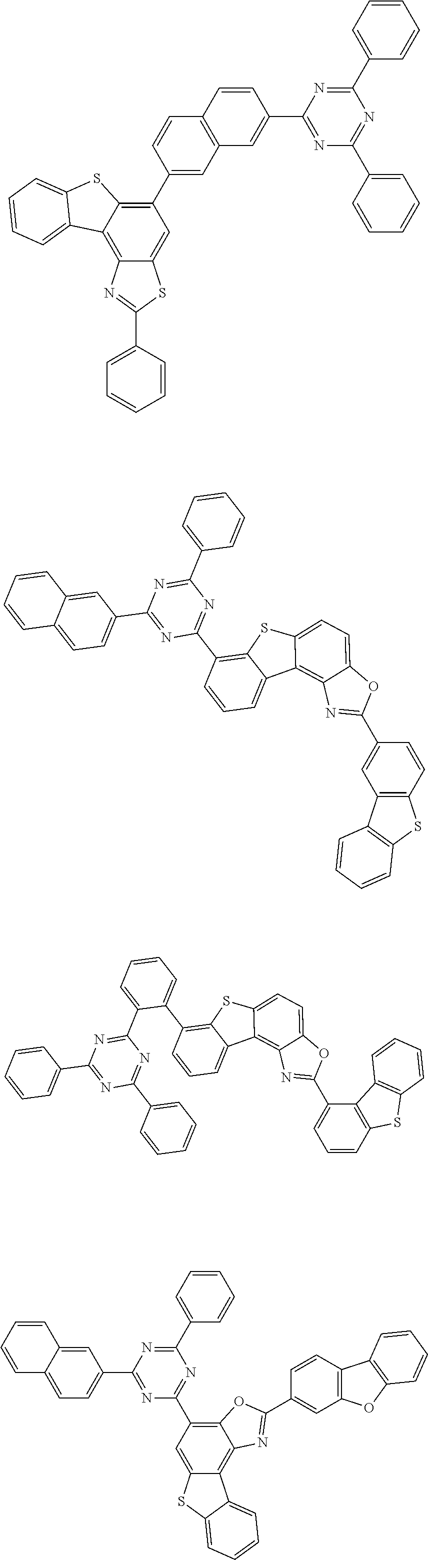 [SMILES: c1ccc(-c2nc(-c3ccccc3)nc(-c3ccc4ccc(-c5cc6sc(-c7ccccc7)nc6c6c5sc5ccccc56)cc4c3)n2)cc1 ; c1ccc(-c2nc(-c3ccc4ccccc4c3)nc(-c3cccc4c3sc3ccc5oc(-c6ccc7sc8ccccc8c7c6)nc5c34)n2)cc1 ; c1ccc(-c2nc(-c3ccccc3)nc(-c3ccccc3-c3cccc4c3sc3ccc5oc(-c6cccc7sc8ccccc8c67)nc5c34)n2)cc1 ; c1ccc(-c2nc(-c3ccc4ccccc4c3)nc(-c3cc4sc5ccccc5c4c4nc(-c5ccc6c(c5)oc5ccccc56)oc34)n2)cc1]

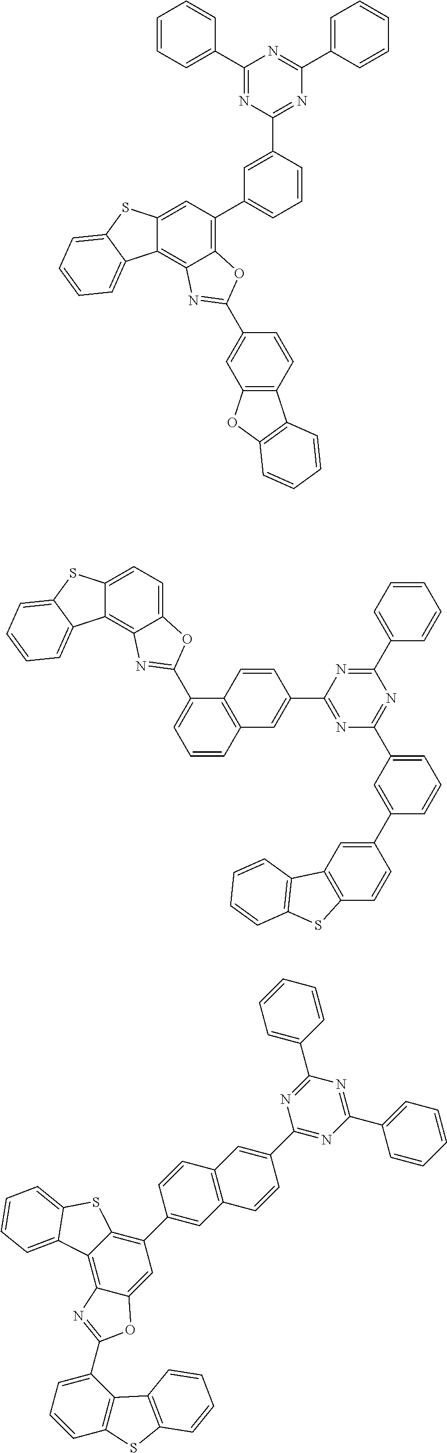 [SMILES: c1ccc(-c2nc(-c3ccccc3)nc(-c3cccc(-c4cc5sc6ccccc6c5c5nc(-c6ccc7c(c6)oc6ccccc67)oc45)c3)n2)cc1 ; c1ccc(-c2nc(-c3cccc(-c4ccc5sc6ccccc6c5c4)c3)nc(-c3ccc4c(-c5nc6c(ccc7sc8ccccc8c76)o5)cccc4c3)n2)cc1 ; c1ccc(-c2nc(-c3ccccc3)nc(-c3ccc4cc(-c5cc6oc(-c7cccc8sc9ccccc9c78)nc6c6c5sc5ccccc56)ccc4c3)n2)cc1]

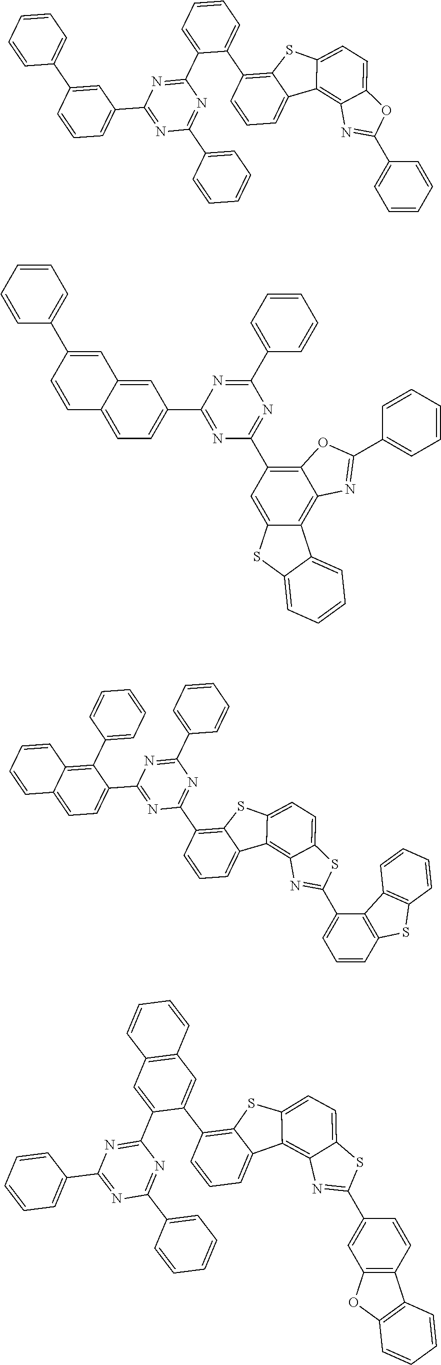 [SMILES: c1ccc(-c2cccc(-c3nc(-c4ccccc4)nc(-c4ccccc4-c4cccc5c4sc4ccc6oc(-c7ccccc7)nc6c45)n3)c2)cc1 ; c1ccc(-c2ccc3ccc(-c4nc(-c5ccccc5)nc(-c5cc6sc7ccccc7c6c6nc(-c7ccccc7)oc56)n4)cc3c2)cc1 ; c1ccc(-c2nc(-c3ccc4ccccc4c3-c3ccccc3)nc(-c3cccc4c3sc3ccc5sc(-c6cccc7sc8ccccc8c67)nc5c34)n2)cc1 ; c1ccc(-c2nc(-c3ccccc3)nc(-c3cc4ccccc4cc3-c3cccc4c3sc3ccc5sc(-c6ccc7c(c6)oc6ccccc67)nc5c34)n2)cc1]

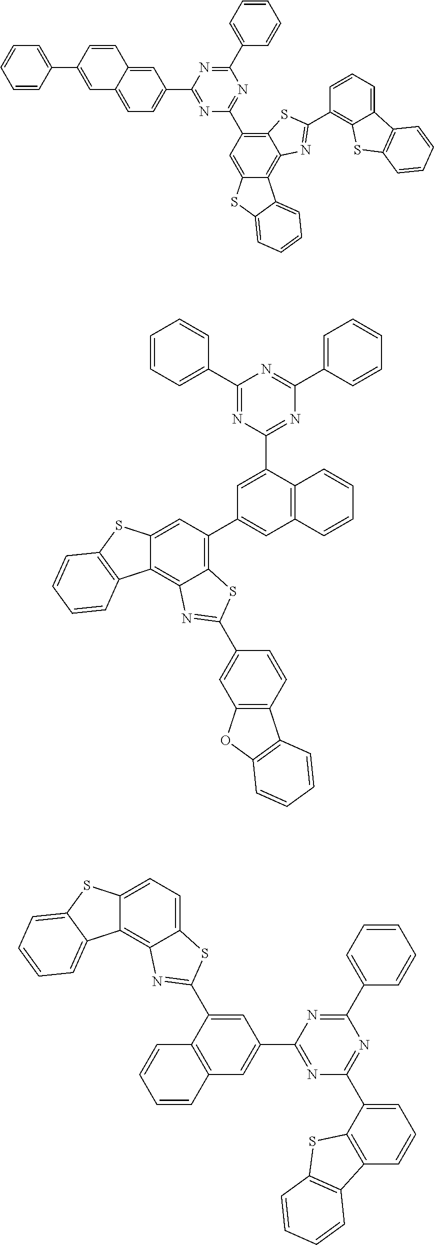 [SMILES: c1ccc(-c2ccc3cc(-c4nc(-c5ccccc5)nc(-c5cc6sc7ccccc7c6c6nc(-c7cccc8c7sc7ccccc78)sc56)n4)ccc3c2)cc1 ; c1ccc(-c2nc(-c3ccccc3)nc(-c3cc(-c4cc5sc6ccccc6c5c5nc(-c6ccc7c(c6)oc6ccccc67)sc45)cc4ccccc34)n2)cc1 ; c1ccc(-c2nc(-c3cc(-c4nc5c(ccc6sc7ccccc7c65)s4)c4ccccc4c3)nc(-c3cccc4c3sc3ccccc34)n2)cc1]

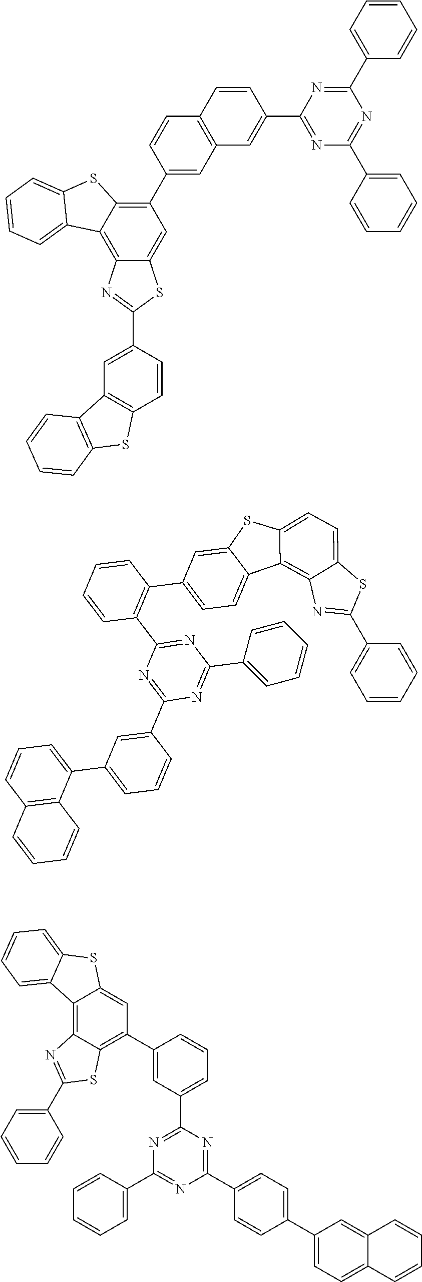 [SMILES: c1ccc(-c2nc(-c3ccccc3)nc(-c3ccc4ccc(-c5cc6sc(-c7ccc8sc9ccccc9c8c7)nc6c6c5sc5ccccc56)cc4c3)n2)cc1 ; c1ccc(-c2nc(-c3cccc(-c4cccc5ccccc45)c3)nc(-c3ccccc3-c3ccc4c(c3)sc3ccc5sc(-c6ccccc6)nc5c34)n2)cc1 ; c1ccc(-c2nc(-c3ccc(-c4ccc5ccccc5c4)cc3)nc(-c3cccc(-c4cc5sc6ccccc6c5c5nc(-c6ccccc6)sc45)c3)n2)cc1]

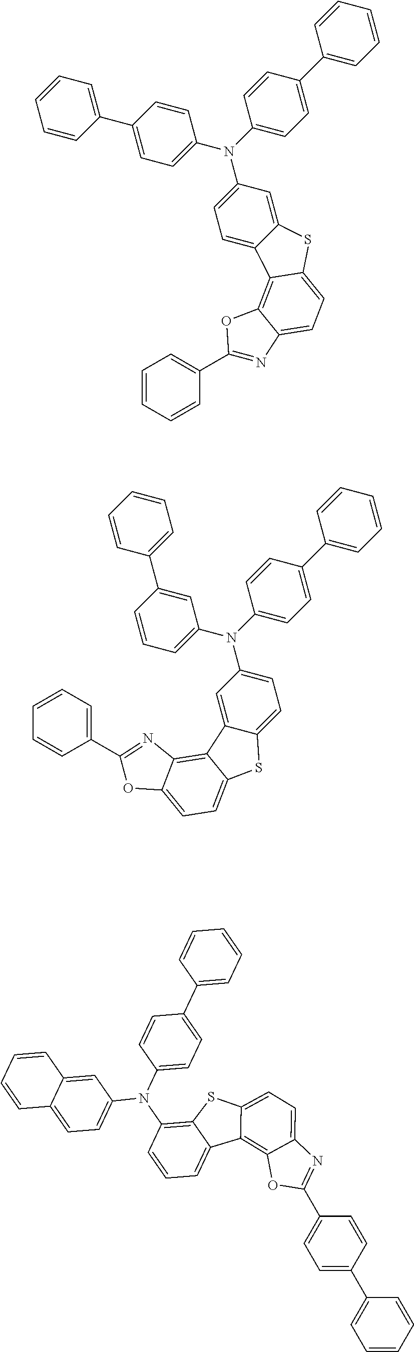 [SMILES: c1ccc(-c2ccc(N(c3ccc(-c4ccccc4)cc3)c3ccc4c(c3)sc3ccc5nc(-c6ccccc6)oc5c34)cc2)cc1 ; c1ccc(-c2ccc(N(c3cccc(-c4ccccc4)c3)c3ccc4sc5ccc6oc(-c7ccccc7)nc6c5c4c3)cc2)cc1 ; c1ccc(-c2ccc(-c3nc4ccc5sc6c(N(c7ccc(-c8ccccc8)cc7)c7ccc8ccccc8c7)cccc6c5c4o3)cc2)cc1]

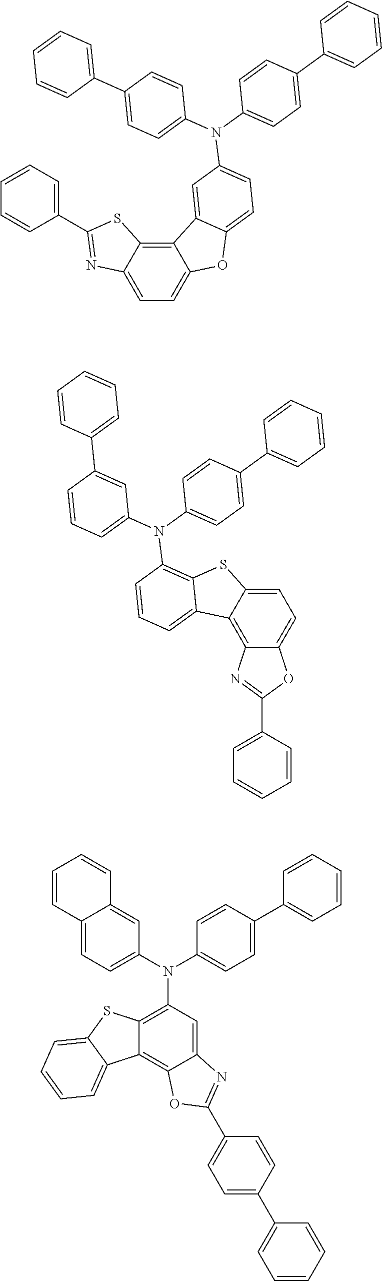 [SMILES: c1ccc(-c2ccc(N(c3ccc(-c4ccccc4)cc3)c3ccc4oc5ccc6nc(-c7ccccc7)sc6c5c4c3)cc2)cc1 ; c1ccc(-c2ccc(N(c3cccc(-c4ccccc4)c3)c3cccc4c3sc3ccc5oc(-c6ccccc6)nc5c34)cc2)cc1 ; c1ccc(-c2ccc(-c3nc4cc(N(c5ccc(-c6ccccc6)cc5)c5ccc6ccccc6c5)c5sc6ccccc6c5c4o3)cc2)cc1]

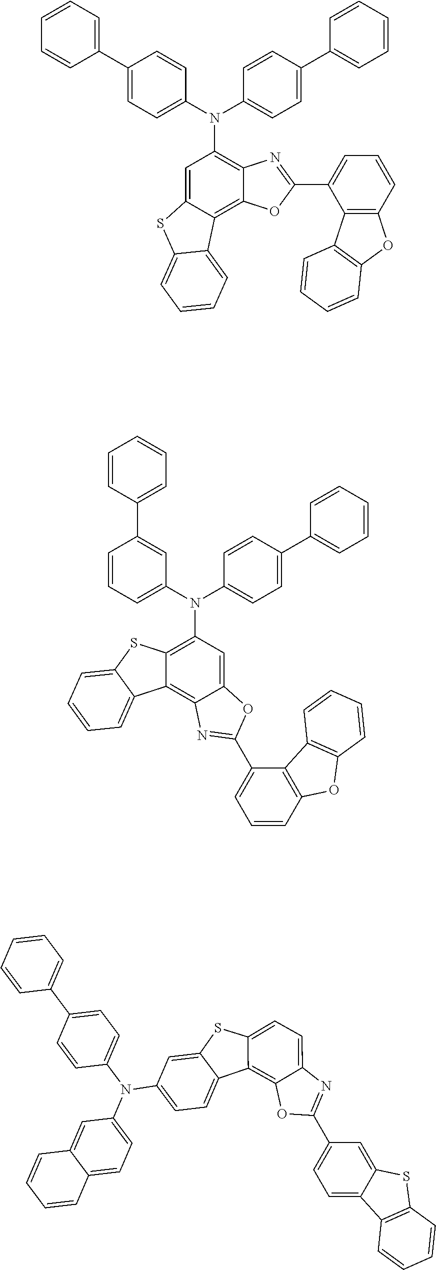 [SMILES: c1ccc(-c2ccc(N(c3ccc(-c4ccccc4)cc3)c3cc4sc5ccccc5c4c4oc(-c5cccc6oc7ccccc7c56)nc34)cc2)cc1 ; c1ccc(-c2ccc(N(c3cccc(-c4ccccc4)c3)c3cc4oc(-c5cccc6oc7ccccc7c56)nc4c4c3sc3ccccc34)cc2)cc1 ; c1ccc(-c2ccc(N(c3ccc4ccccc4c3)c3ccc4c(c3)sc3ccc5nc(-c6ccc7c(c6)sc6ccccc67)oc5c34)cc2)cc1]

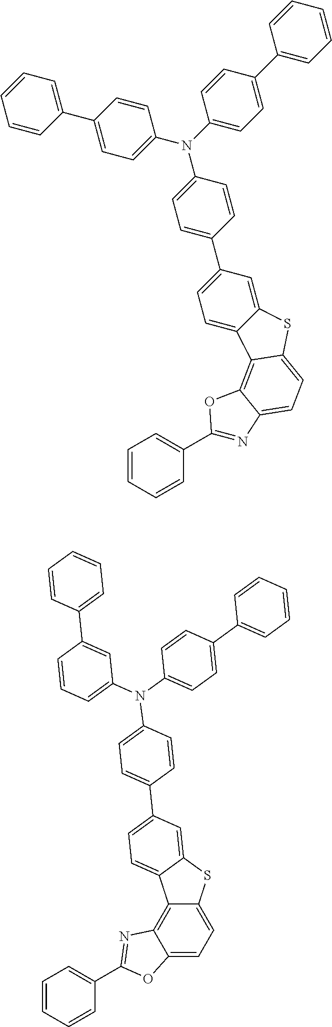 [SMILES: c1ccc(-c2ccc(N(c3ccc(-c4ccccc4)cc3)c3ccc(-c4ccc5c(c4)sc4ccc6nc(-c7ccccc7)oc6c45)cc3)cc2)cc1 ; c1ccc(-c2ccc(N(c3ccc(-c4ccc5c(c4)sc4ccc6oc(-c7ccccc7)nc6c45)cc3)c3cccc(-c4ccccc4)c3)cc2)cc1]

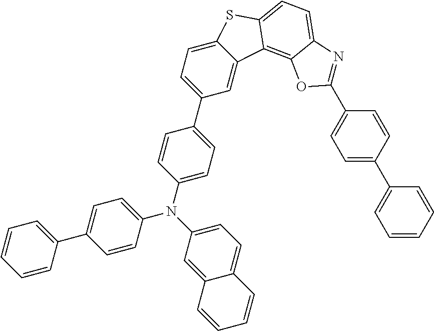 [SMILES: c1ccc(-c2ccc(-c3nc4ccc5sc6ccc(-c7ccc(N(c8ccc(-c9ccccc9)cc8)c8ccc9ccccc9c8)cc7)cc6c5c4o3)cc2)cc1]

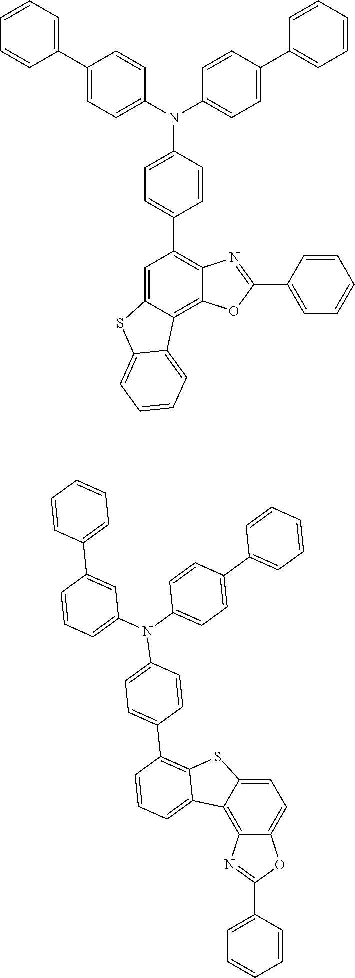 [SMILES: c1ccc(-c2ccc(N(c3ccc(-c4ccccc4)cc3)c3ccc(-c4cc5sc6ccccc6c5c5oc(-c6ccccc6)nc45)cc3)cc2)cc1 ; c1ccc(-c2ccc(N(c3ccc(-c4cccc5c4sc4ccc6oc(-c7ccccc7)nc6c45)cc3)c3cccc(-c4ccccc4)c3)cc2)cc1]

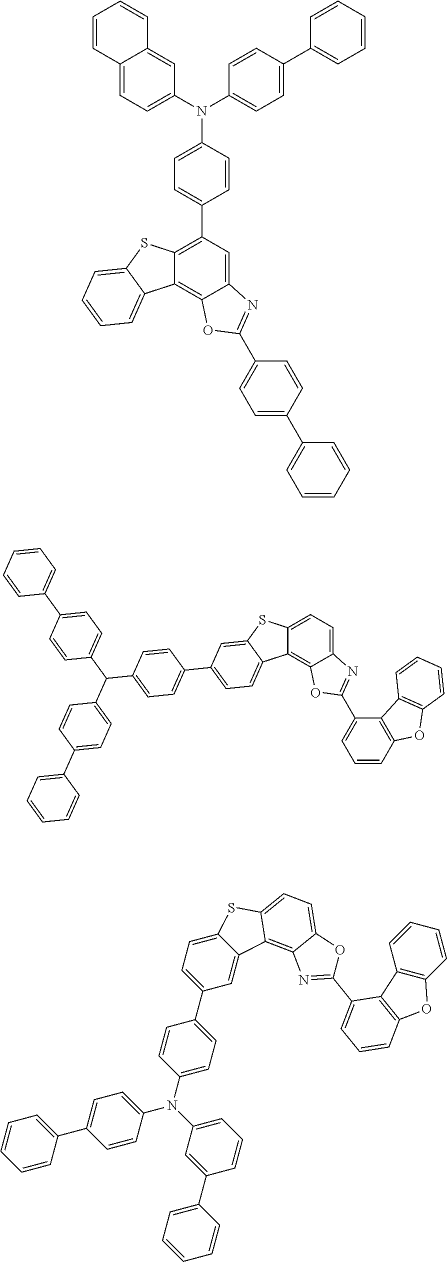 [SMILES: c1ccc(-c2ccc(-c3nc4cc(-c5ccc(N(c6ccc(-c7ccccc7)cc6)c6ccc7ccccc7c6)cc5)c5sc6ccccc6c5c4o3)cc2)cc1 ; c1ccc(-c2ccc(C(c3ccc(-c4ccccc4)cc3)c3ccc(-c4ccc5c(c4)sc4ccc6nc(-c7cccc8oc9ccccc9c78)oc6c45)cc3)cc2)cc1 ; c1ccc(-c2ccc(N(c3ccc(-c4ccc5sc6ccc7oc(-c8cccc9oc%10ccccc%10c89)nc7c6c5c4)cc3)c3cccc(-c4ccccc4)c3)cc2)cc1]

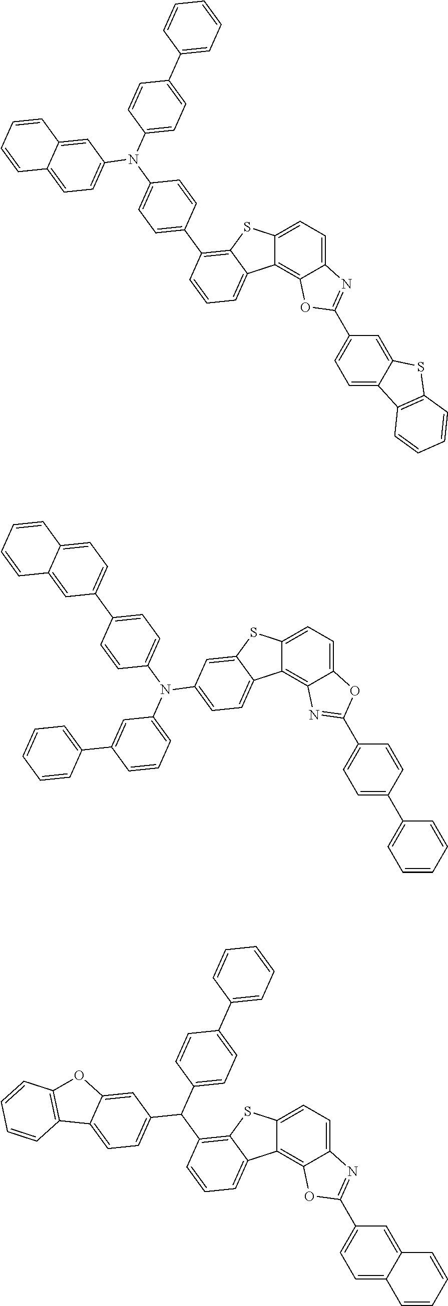 [SMILES: c1ccc(-c2ccc(N(c3ccc(-c4cccc5c4sc4ccc6nc(-c7ccc8c(c7)sc7ccccc78)oc6c45)cc3)c3ccc4ccccc4c3)cc2)cc1 ; c1ccc(-c2ccc(-c3nc4c(ccc5sc6cc(N(c7ccc(-c8ccc9ccccc9c8)cc7)c7cccc(-c8ccccc8)c7)ccc6c54)o3)cc2)cc1 ; c1ccc(-c2ccc(C(c3ccc4c(c3)oc3ccccc34)c3cccc4c3sc3ccc5nc(-c6ccc7ccccc7c6)oc5c34)cc2)cc1]

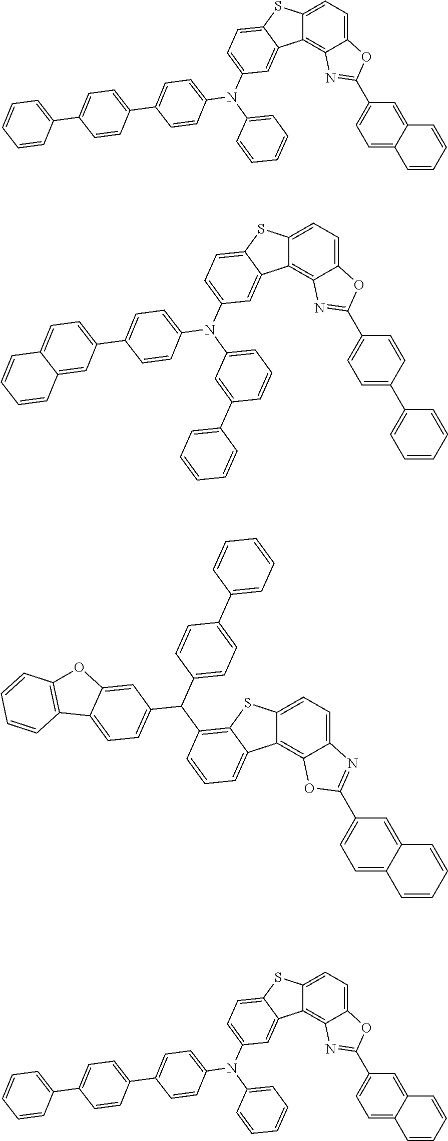 [SMILES: c1ccc(-c2ccc(-c3ccc(N(c4ccccc4)c4ccc5sc6ccc7oc(-c8ccc9ccccc9c8)nc7c6c5c4)cc3)cc2)cc1 ; c1ccc(-c2ccc(-c3nc4c(ccc5sc6ccc(N(c7ccc(-c8ccc9ccccc9c8)cc7)c7cccc(-c8ccccc8)c7)cc6c54)o3)cc2)cc1 ; c1ccc(-c2ccc(C(c3ccc4c(c3)oc3ccccc34)c3cccc4c3sc3ccc5nc(-c6ccc7ccccc7c6)oc5c34)cc2)cc1 ; c1ccc(-c2ccc(-c3ccc(N(c4ccccc4)c4ccc5sc6ccc7oc(-c8ccc9ccccc9c8)nc7c6c5c4)cc3)cc2)cc1]

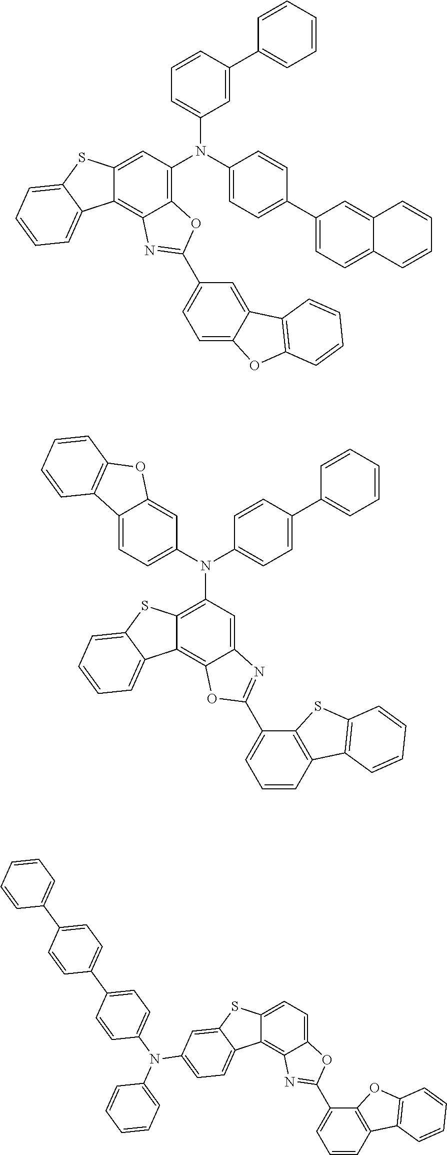 [SMILES: c1ccc(-c2cccc(N(c3ccc(-c4ccc5ccccc5c4)cc3)c3cc4sc5ccccc5c4c4nc(-c5ccc6oc7ccccc7c6c5)oc34)c2)cc1 ; c1ccc(-c2ccc(N(c3ccc4c(c3)oc3ccccc34)c3cc4nc(-c5cccc6c5sc5ccccc56)oc4c4c3sc3ccccc34)cc2)cc1 ; c1ccc(-c2ccc(-c3ccc(N(c4ccccc4)c4ccc5c(c4)sc4ccc6oc(-c7cccc8c7oc7ccccc78)nc6c45)cc3)cc2)cc1]

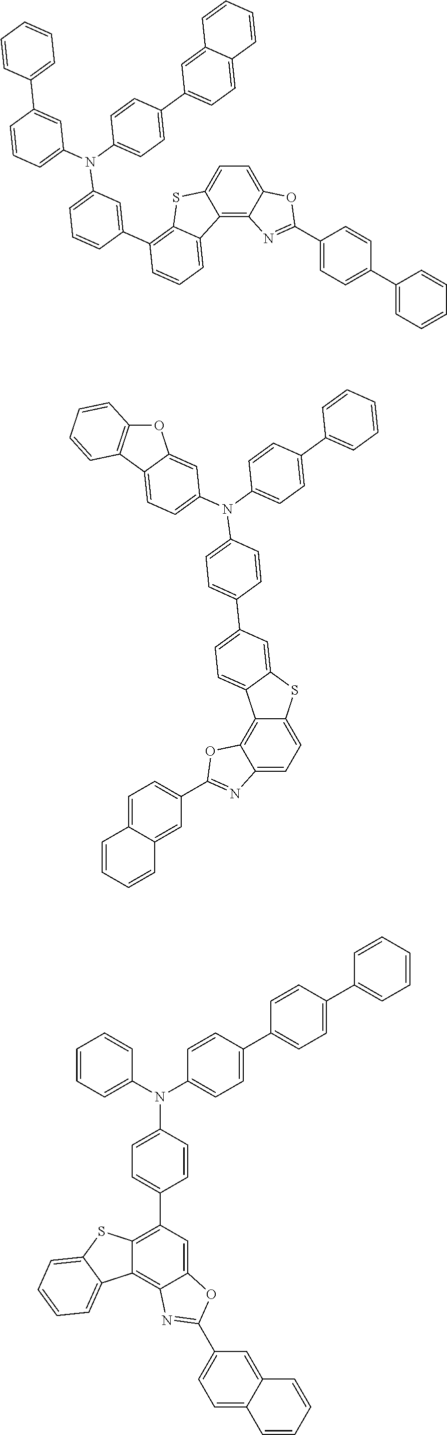 [SMILES: c1ccc(-c2ccc(-c3nc4c(ccc5sc6c(-c7cccc(N(c8ccc(-c9ccc%10ccccc%10c9)cc8)c8cccc(-c9ccccc9)c8)c7)cccc6c54)o3)cc2)cc1 ; c1ccc(-c2ccc(N(c3ccc(-c4ccc5c(c4)sc4ccc6nc(-c7ccc8ccccc8c7)oc6c45)cc3)c3ccc4c(c3)oc3ccccc34)cc2)cc1 ; c1ccc(-c2ccc(-c3ccc(N(c4ccccc4)c4ccc(-c5cc6oc(-c7ccc8ccccc8c7)nc6c6c5sc5ccccc56)cc4)cc3)cc2)cc1]

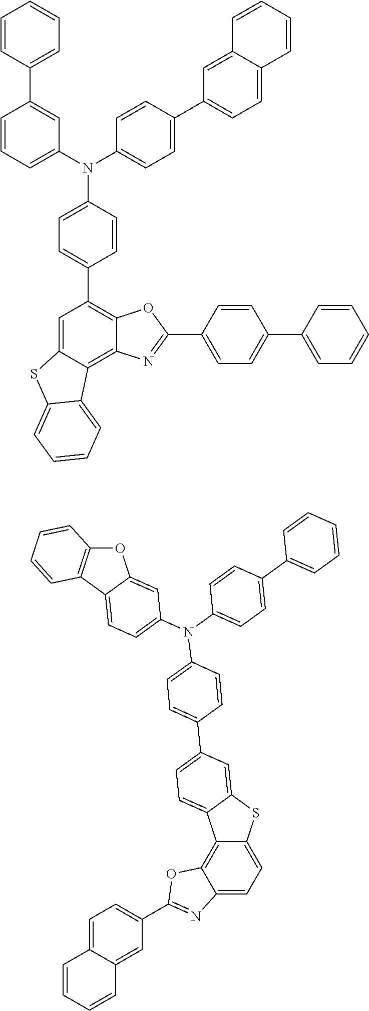 [SMILES: c1ccc(-c2ccc(-c3nc4c(o3)c(-c3ccc(N(c5ccc(-c6ccc7ccccc7c6)cc5)c5cccc(-c6ccccc6)c5)cc3)cc3sc5ccccc5c34)cc2)cc1 ; c1ccc(-c2ccc(N(c3ccc(-c4ccc5c(c4)sc4ccc6nc(-c7ccc8ccccc8c7)oc6c45)cc3)c3ccc4c(c3)oc3ccccc34)cc2)cc1]

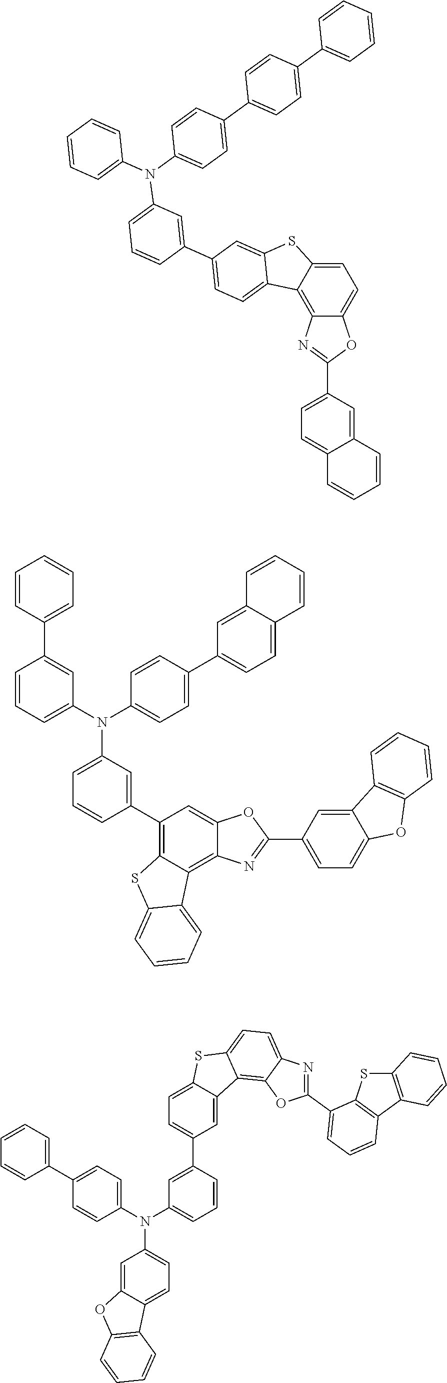 [SMILES: c1ccc(-c2ccc(-c3ccc(N(c4ccccc4)c4cccc(-c5ccc6c(c5)sc5ccc7oc(-c8ccc9ccccc9c8)nc7c56)c4)cc3)cc2)cc1 ; c1ccc(-c2cccc(N(c3ccc(-c4ccc5ccccc5c4)cc3)c3cccc(-c4cc5oc(-c6ccc7oc8ccccc8c7c6)nc5c5c4sc4ccccc45)c3)c2)cc1 ; c1ccc(-c2ccc(N(c3cccc(-c4ccc5sc6ccc7nc(-c8cccc9c8sc8ccccc89)oc7c6c5c4)c3)c3ccc4c(c3)oc3ccccc34)cc2)cc1]

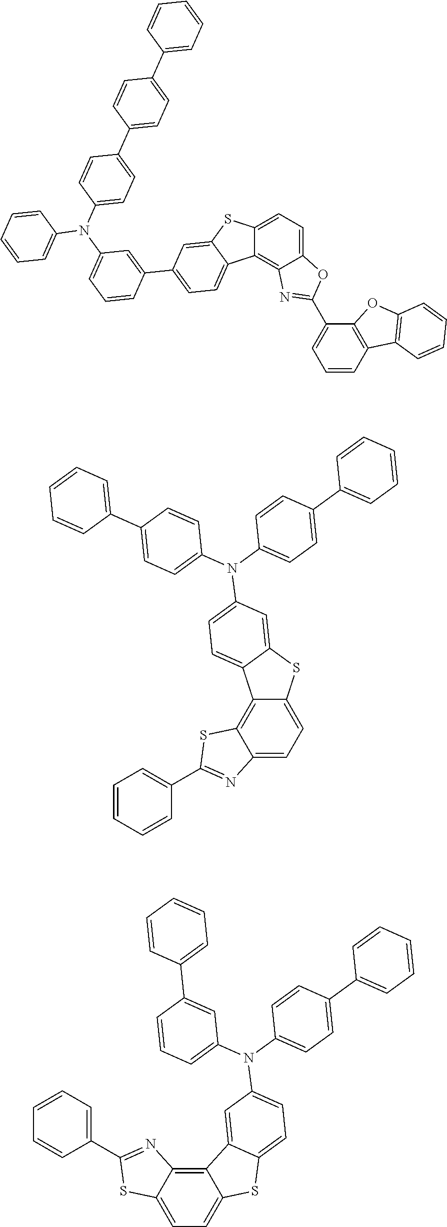 [SMILES: c1ccc(-c2ccc(-c3ccc(N(c4ccccc4)c4cccc(-c5ccc6c(c5)sc5ccc7oc(-c8cccc9c8oc8ccccc89)nc7c56)c4)cc3)cc2)cc1 ; c1ccc(-c2ccc(N(c3ccc(-c4ccccc4)cc3)c3ccc4c(c3)sc3ccc5nc(-c6ccccc6)sc5c34)cc2)cc1 ; c1ccc(-c2ccc(N(c3cccc(-c4ccccc4)c3)c3ccc4sc5ccc6sc(-c7ccccc7)nc6c5c4c3)cc2)cc1]

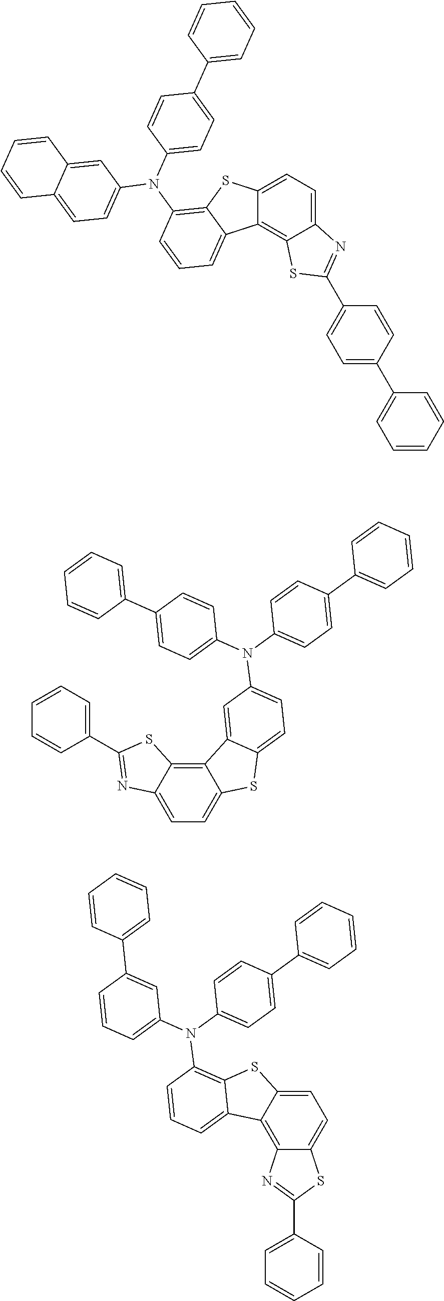 [SMILES: c1ccc(-c2ccc(-c3nc4ccc5sc6c(N(c7ccc(-c8ccccc8)cc7)c7ccc8ccccc8c7)cccc6c5c4s3)cc2)cc1 ; c1ccc(-c2ccc(N(c3ccc(-c4ccccc4)cc3)c3ccc4sc5ccc6nc(-c7ccccc7)sc6c5c4c3)cc2)cc1 ; c1ccc(-c2ccc(N(c3cccc(-c4ccccc4)c3)c3cccc4c3sc3ccc5sc(-c6ccccc6)nc5c34)cc2)cc1]

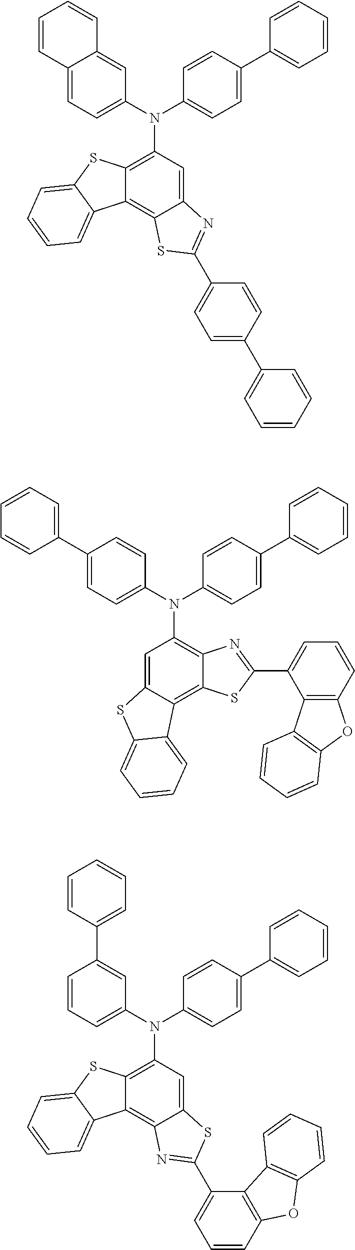 [SMILES: c1ccc(-c2ccc(-c3nc4cc(N(c5ccc(-c6ccccc6)cc5)c5ccc6ccccc6c5)c5sc6ccccc6c5c4s3)cc2)cc1 ; c1ccc(-c2ccc(N(c3ccc(-c4ccccc4)cc3)c3cc4sc5ccccc5c4c4sc(-c5cccc6oc7ccccc7c56)nc34)cc2)cc1 ; c1ccc(-c2ccc(N(c3cccc(-c4ccccc4)c3)c3cc4sc(-c5cccc6oc7ccccc7c56)nc4c4c3sc3ccccc34)cc2)cc1]

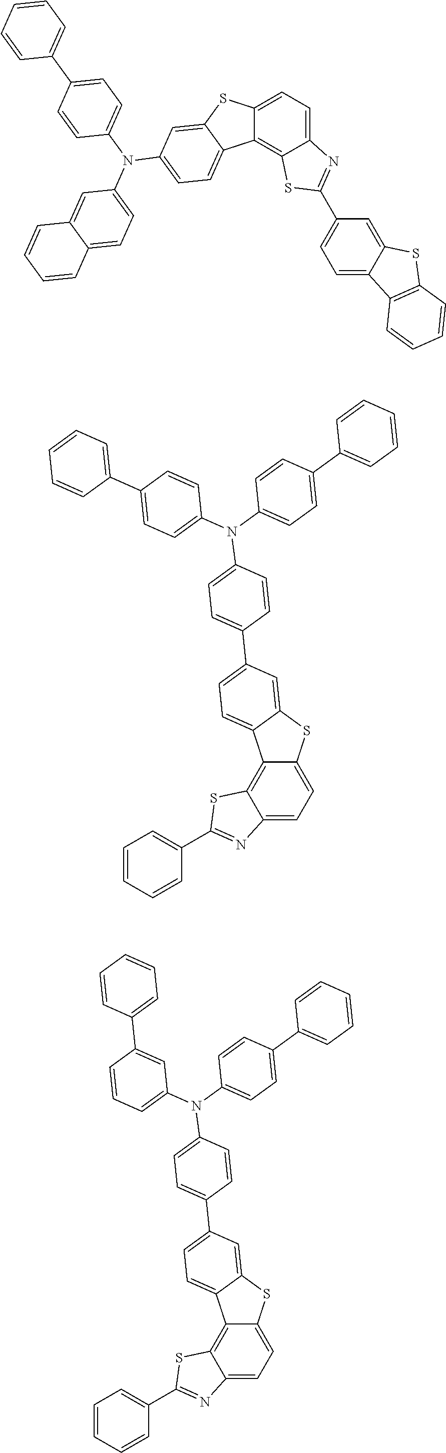 [SMILES: c1ccc(-c2ccc(N(c3ccc4ccccc4c3)c3ccc4c(c3)sc3ccc5nc(-c6ccc7c(c6)sc6ccccc67)sc5c34)cc2)cc1 ; c1ccc(-c2ccc(N(c3ccc(-c4ccccc4)cc3)c3ccc(-c4ccc5c(c4)sc4ccc6nc(-c7ccccc7)sc6c45)cc3)cc2)cc1 ; c1ccc(-c2ccc(N(c3ccc(-c4ccc5c(c4)sc4ccc6nc(-c7ccccc7)sc6c45)cc3)c3cccc(-c4ccccc4)c3)cc2)cc1]

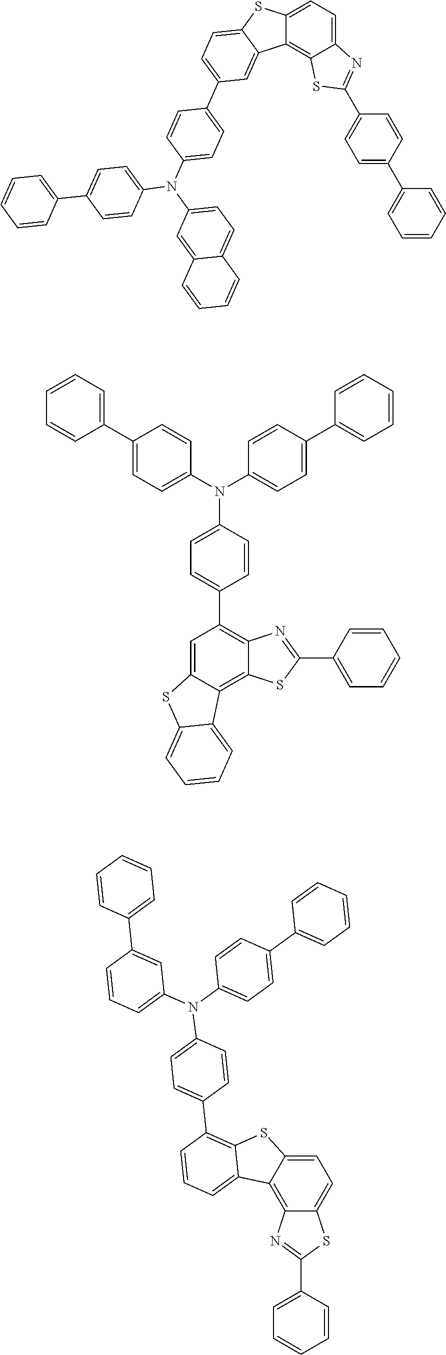 [SMILES: c1ccc(-c2ccc(-c3nc4ccc5sc6ccc(-c7ccc(N(c8ccc(-c9ccccc9)cc8)c8ccc9ccccc9c8)cc7)cc6c5c4s3)cc2)cc1 ; c1ccc(-c2ccc(N(c3ccc(-c4ccccc4)cc3)c3ccc(-c4cc5sc6ccccc6c5c5sc(-c6ccccc6)nc45)cc3)cc2)cc1 ; c1ccc(-c2ccc(N(c3ccc(-c4cccc5c4sc4ccc6sc(-c7ccccc7)nc6c45)cc3)c3cccc(-c4ccccc4)c3)cc2)cc1]

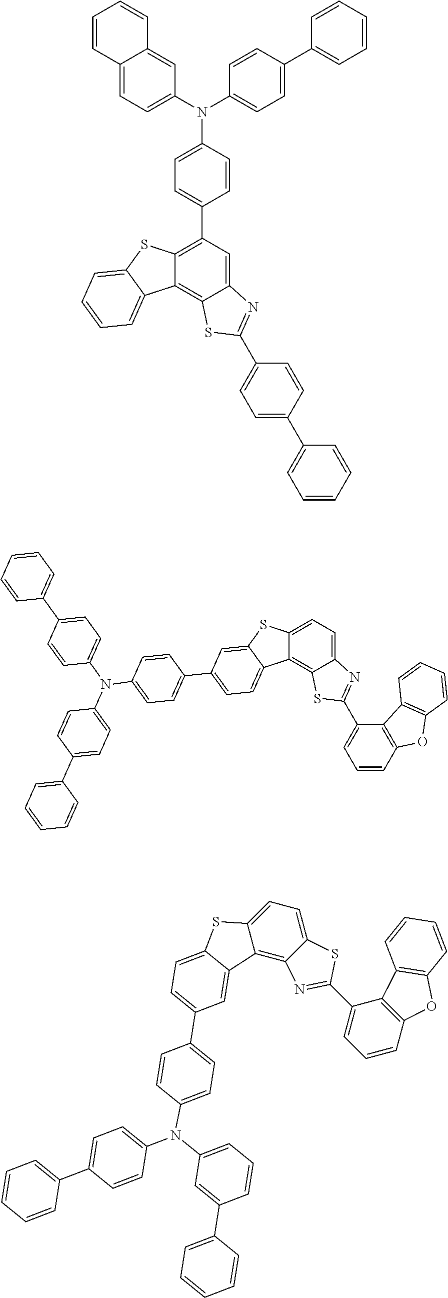 [SMILES: c1ccc(-c2ccc(-c3nc4cc(-c5ccc(N(c6ccc(-c7ccccc7)cc6)c6ccc7ccccc7c6)cc5)c5sc6ccccc6c5c4s3)cc2)cc1 ; c1ccc(-c2ccc(N(c3ccc(-c4ccccc4)cc3)c3ccc(-c4ccc5c(c4)sc4ccc6nc(-c7cccc8oc9ccccc9c78)sc6c45)cc3)cc2)cc1 ; c1ccc(-c2ccc(N(c3ccc(-c4ccc5sc6ccc7sc(-c8cccc9oc%10ccccc%10c89)nc7c6c5c4)cc3)c3cccc(-c4ccccc4)c3)cc2)cc1]

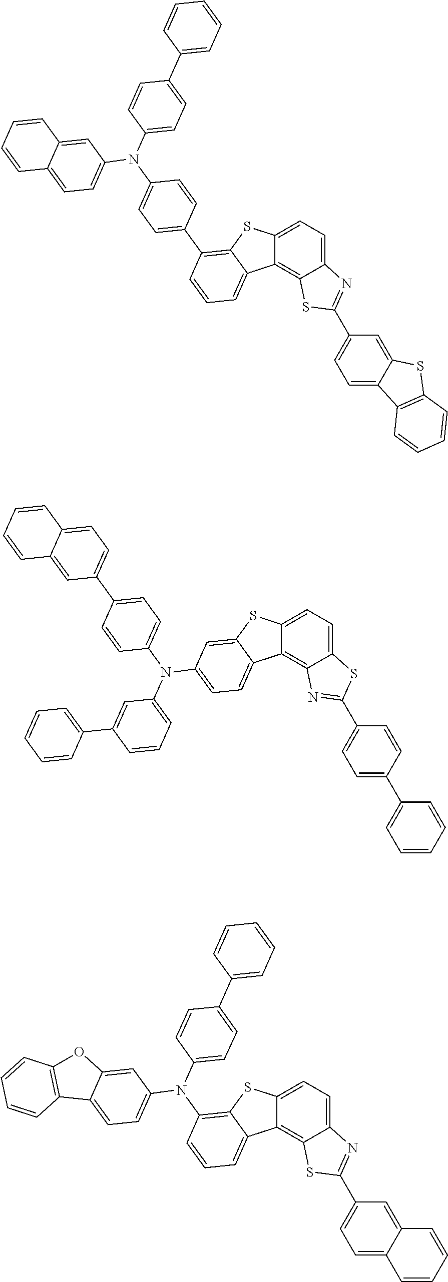 [SMILES: c1ccc(-c2ccc(N(c3ccc(-c4cccc5c4sc4ccc6nc(-c7ccc8c(c7)sc7ccccc78)sc6c45)cc3)c3ccc4ccccc4c3)cc2)cc1 ; c1ccc(-c2ccc(-c3nc4c(ccc5sc6cc(N(c7ccc(-c8ccc9ccccc9c8)cc7)c7cccc(-c8ccccc8)c7)ccc6c54)s3)cc2)cc1 ; c1ccc(-c2ccc(N(c3ccc4c(c3)oc3ccccc34)c3cccc4c3sc3ccc5nc(-c6ccc7ccccc7c6)sc5c34)cc2)cc1]

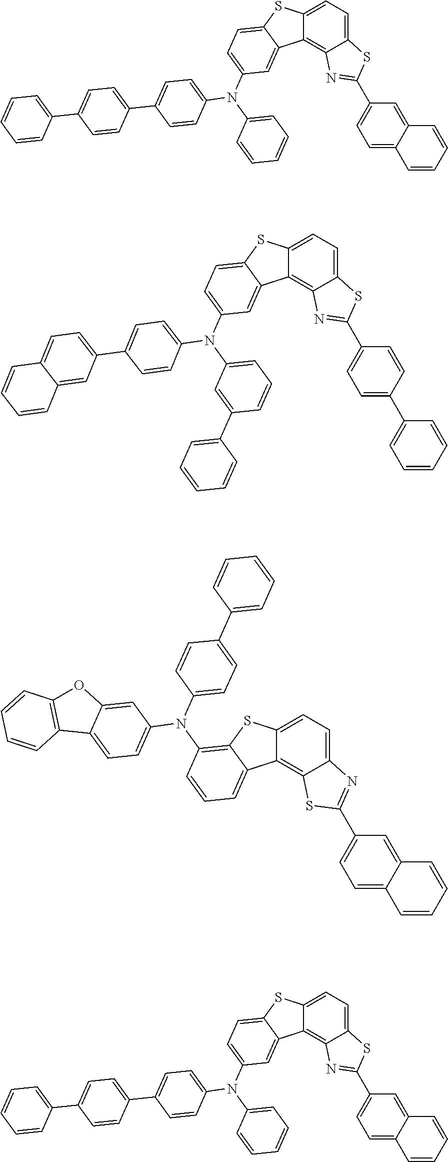 [SMILES: c1ccc(-c2ccc(-c3ccc(N(c4ccccc4)c4ccc5sc6ccc7sc(-c8ccc9ccccc9c8)nc7c6c5c4)cc3)cc2)cc1 ; c1ccc(-c2ccc(-c3nc4c(ccc5sc6ccc(N(c7ccc(-c8ccc9ccccc9c8)cc7)c7cccc(-c8ccccc8)c7)cc6c54)s3)cc2)cc1 ; c1ccc(-c2ccc(N(c3ccc4c(c3)oc3ccccc34)c3cccc4c3sc3ccc5nc(-c6ccc7ccccc7c6)sc5c34)cc2)cc1 ; c1ccc(-c2ccc(-c3ccc(N(c4ccccc4)c4ccc5sc6ccc7sc(-c8ccc9ccccc9c8)nc7c6c5c4)cc3)cc2)cc1]

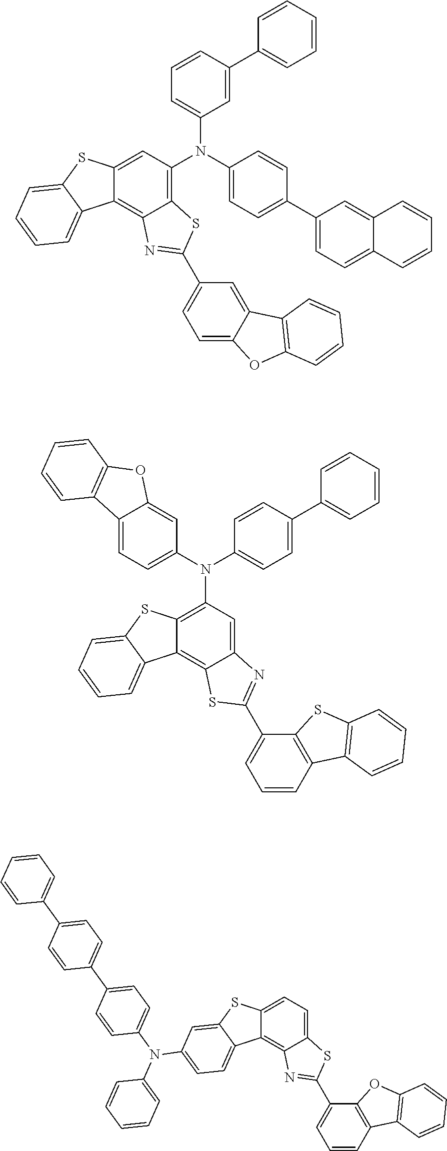 [SMILES: c1ccc(-c2cccc(N(c3ccc(-c4ccc5ccccc5c4)cc3)c3cc4sc5ccccc5c4c4nc(-c5ccc6oc7ccccc7c6c5)sc34)c2)cc1 ; c1ccc(-c2ccc(N(c3ccc4c(c3)oc3ccccc34)c3cc4nc(-c5cccc6c5sc5ccccc56)sc4c4c3sc3ccccc34)cc2)cc1 ; c1ccc(-c2ccc(-c3ccc(N(c4ccccc4)c4ccc5c(c4)sc4ccc6sc(-c7cccc8c7oc7ccccc78)nc6c45)cc3)cc2)cc1]

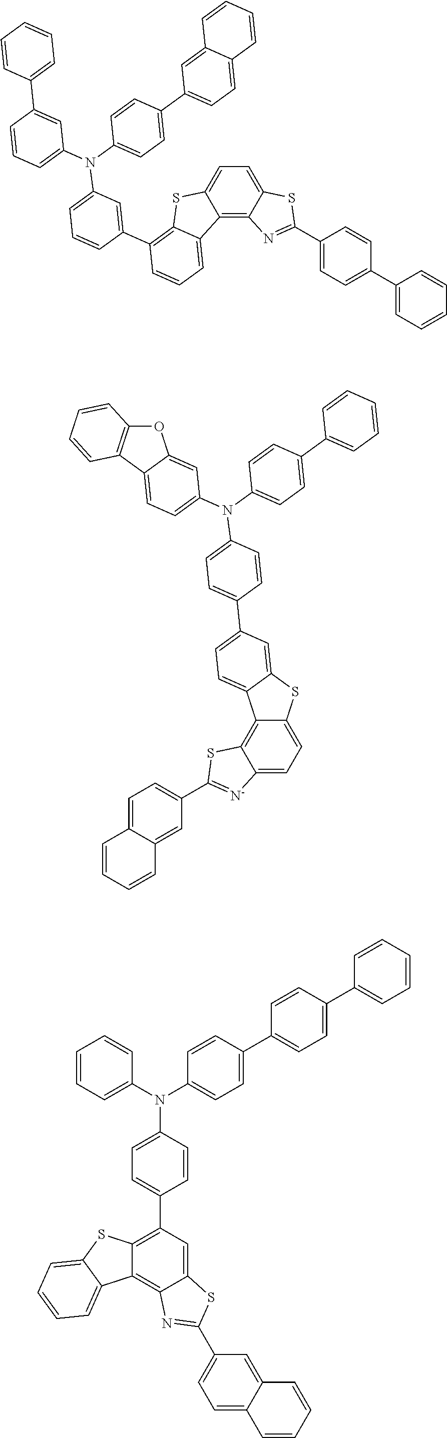

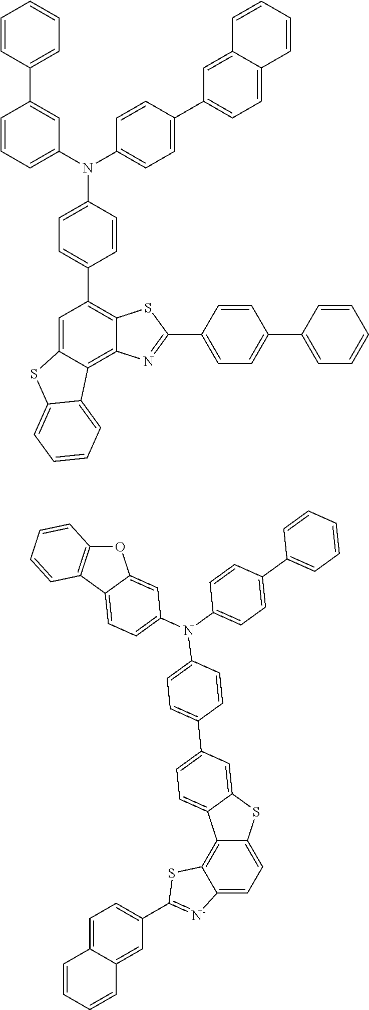

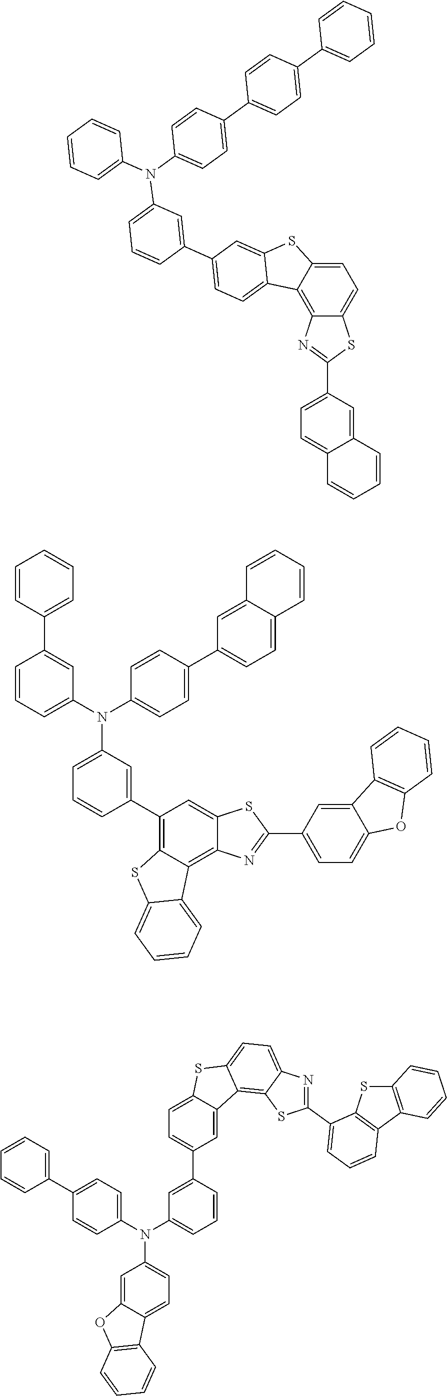 [SMILES: c1ccc(-c2ccc(-c3ccc(N(c4ccccc4)c4cccc(-c5ccc6c(c5)sc5ccc7sc(-c8ccc9ccccc9c8)nc7c56)c4)cc3)cc2)cc1 ; c1ccc(-c2cccc(N(c3ccc(-c4ccc5ccccc5c4)cc3)c3cccc(-c4cc5sc(-c6ccc7oc8ccccc8c7c6)nc5c5c4sc4ccccc45)c3)c2)cc1 ; c1ccc(-c2ccc(N(c3cccc(-c4ccc5sc6ccc7nc(-c8cccc9c8sc8ccccc89)sc7c6c5c4)c3)c3ccc4c(c3)oc3ccccc34)cc2)cc1]

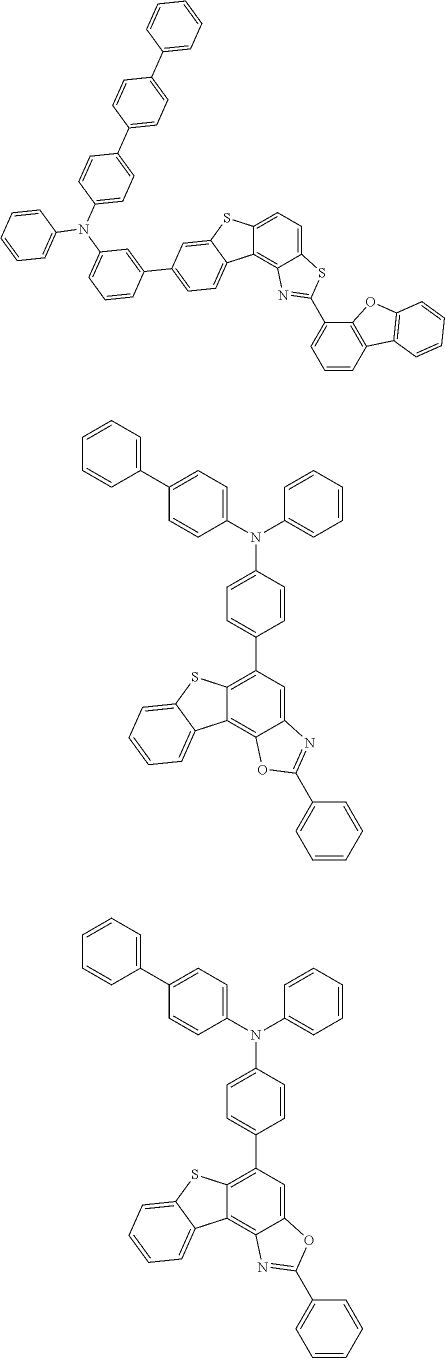 [SMILES: c1ccc(-c2ccc(-c3ccc(N(c4ccccc4)c4cccc(-c5ccc6c(c5)sc5ccc7sc(-c8cccc9c8oc8ccccc89)nc7c56)c4)cc3)cc2)cc1 ; c1ccc(-c2ccc(N(c3ccccc3)c3ccc(-c4cc5nc(-c6ccccc6)oc5c5c4sc4ccccc45)cc3)cc2)cc1 ; c1ccc(-c2ccc(N(c3ccccc3)c3ccc(-c4cc5oc(-c6ccccc6)nc5c5c4sc4ccccc45)cc3)cc2)cc1]

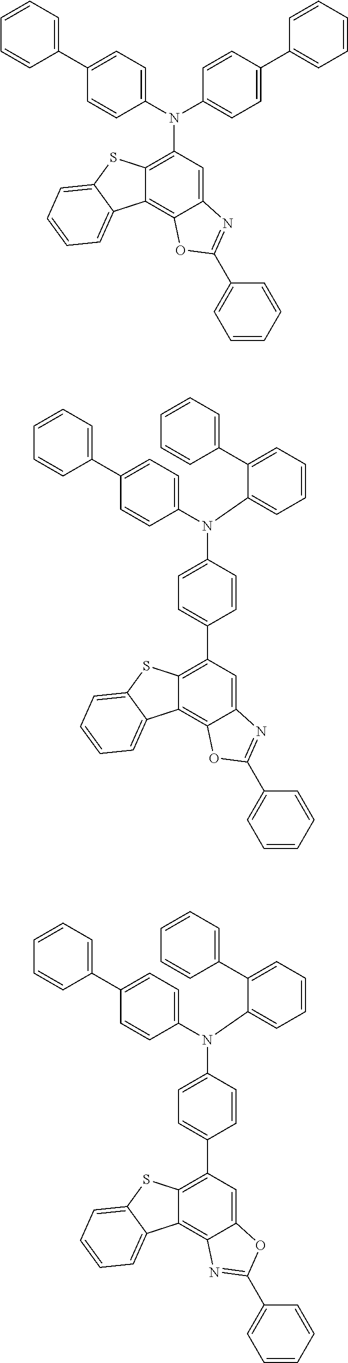 [SMILES: c1ccc(-c2ccc(N(c3ccc(-c4ccccc4)cc3)c3cc4nc(-c5ccccc5)oc4c4c3sc3ccccc34)cc2)cc1 ; c1ccc(-c2ccc(N(c3ccc(-c4cc5nc(-c6ccccc6)oc5c5c4sc4ccccc45)cc3)c3ccccc3-c3ccccc3)cc2)cc1 ; c1ccc(-c2ccc(N(c3ccc(-c4cc5oc(-c6ccccc6)nc5c5c4sc4ccccc45)cc3)c3ccccc3-c3ccccc3)cc2)cc1]

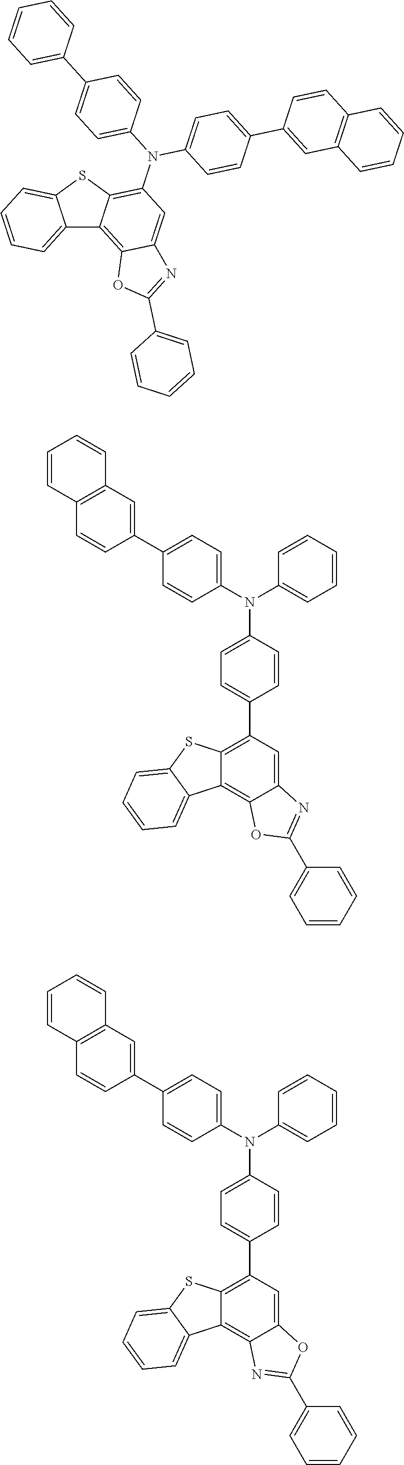 [SMILES: c1ccc(-c2ccc(N(c3ccc(-c4ccc5ccccc5c4)cc3)c3cc4nc(-c5ccccc5)oc4c4c3sc3ccccc34)cc2)cc1 ; c1ccc(-c2nc3cc(-c4ccc(N(c5ccccc5)c5ccc(-c6ccc7ccccc7c6)cc5)cc4)c4sc5ccccc5c4c3o2)cc1 ; c1ccc(-c2nc3c(cc(-c4ccc(N(c5ccccc5)c5ccc(-c6ccc7ccccc7c6)cc5)cc4)c4sc5ccccc5c43)o2)cc1]

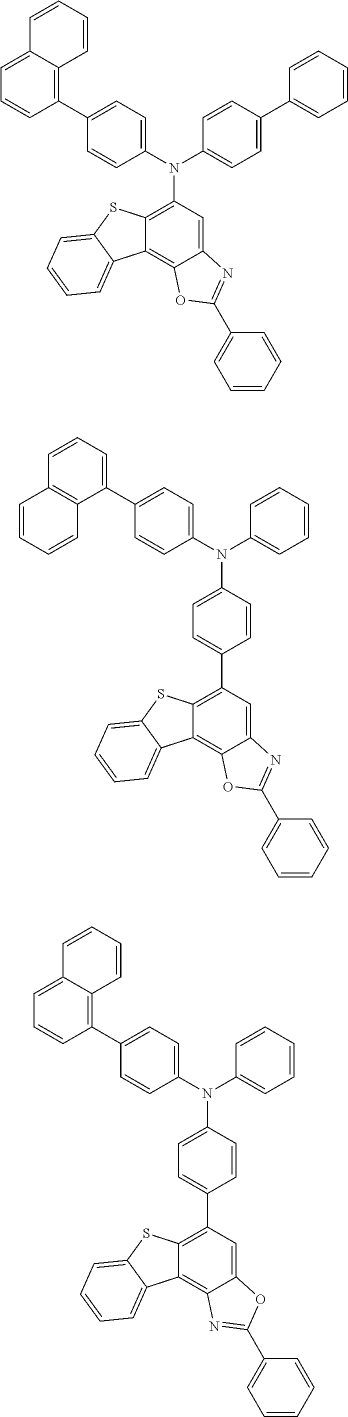 [SMILES: c1ccc(-c2ccc(N(c3ccc(-c4cccc5ccccc45)cc3)c3cc4nc(-c5ccccc5)oc4c4c3sc3ccccc34)cc2)cc1 ; c1ccc(-c2nc3cc(-c4ccc(N(c5ccccc5)c5ccc(-c6cccc7ccccc67)cc5)cc4)c4sc5ccccc5c4c3o2)cc1 ; c1ccc(-c2nc3c(cc(-c4ccc(N(c5ccccc5)c5ccc(-c6cccc7ccccc67)cc5)cc4)c4sc5ccccc5c43)o2)cc1]

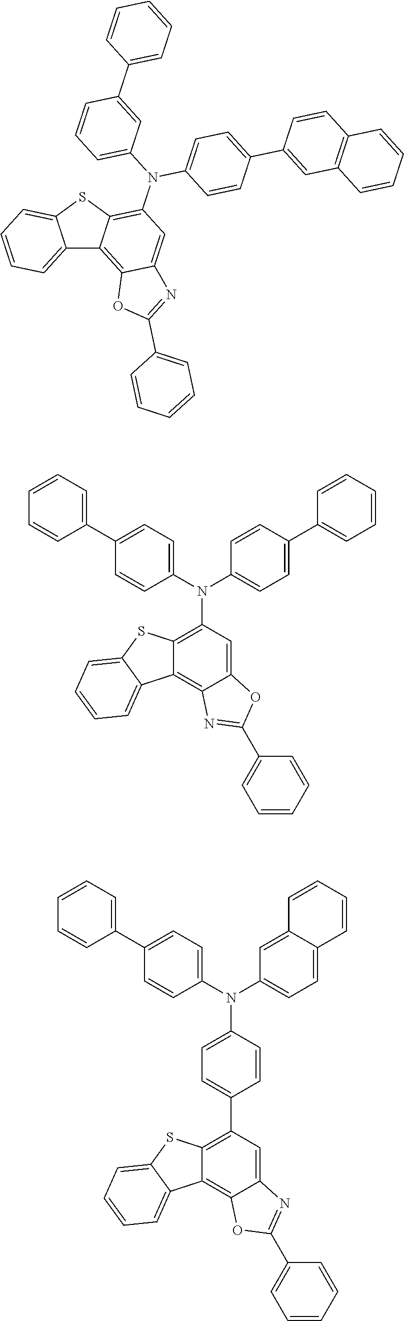 [SMILES: c1ccc(-c2cccc(N(c3ccc(-c4ccc5ccccc5c4)cc3)c3cc4nc(-c5ccccc5)oc4c4c3sc3ccccc34)c2)cc1 ; c1ccc(-c2ccc(N(c3ccc(-c4ccccc4)cc3)c3cc4oc(-c5ccccc5)nc4c4c3sc3ccccc34)cc2)cc1 ; c1ccc(-c2ccc(N(c3ccc(-c4cc5nc(-c6ccccc6)oc5c5c4sc4ccccc45)cc3)c3ccc4ccccc4c3)cc2)cc1]

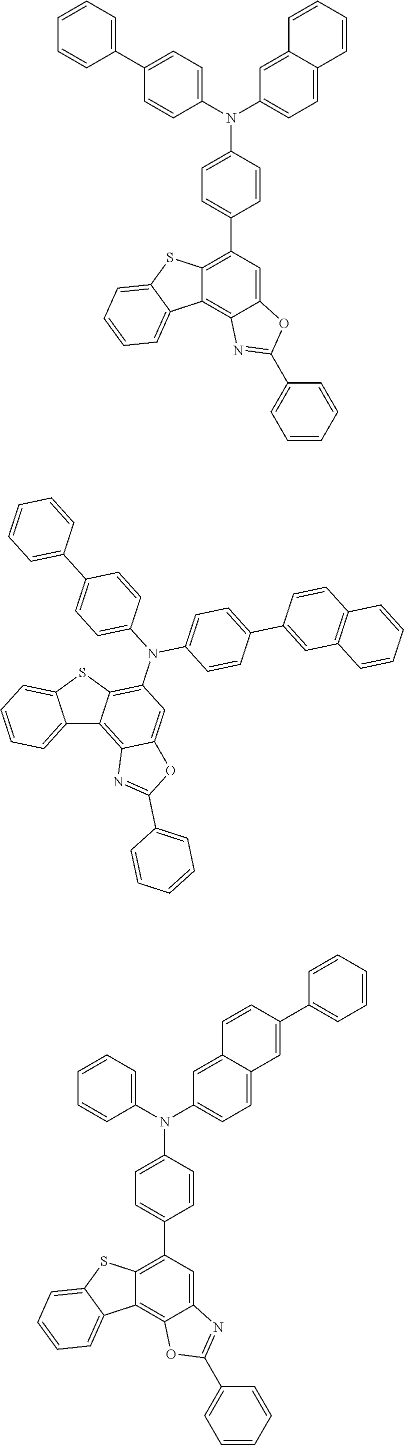 [SMILES: c1ccc(-c2ccc(N(c3ccc(-c4cc5oc(-c6ccccc6)nc5c5c4sc4ccccc45)cc3)c3ccc4ccccc4c3)cc2)cc1 ; c1ccc(-c2ccc(N(c3ccc(-c4ccc5ccccc5c4)cc3)c3cc4oc(-c5ccccc5)nc4c4c3sc3ccccc34)cc2)cc1 ; c1ccc(-c2ccc3cc(N(c4ccccc4)c4ccc(-c5cc6nc(-c7ccccc7)oc6c6c5sc5ccccc56)cc4)ccc3c2)cc1]

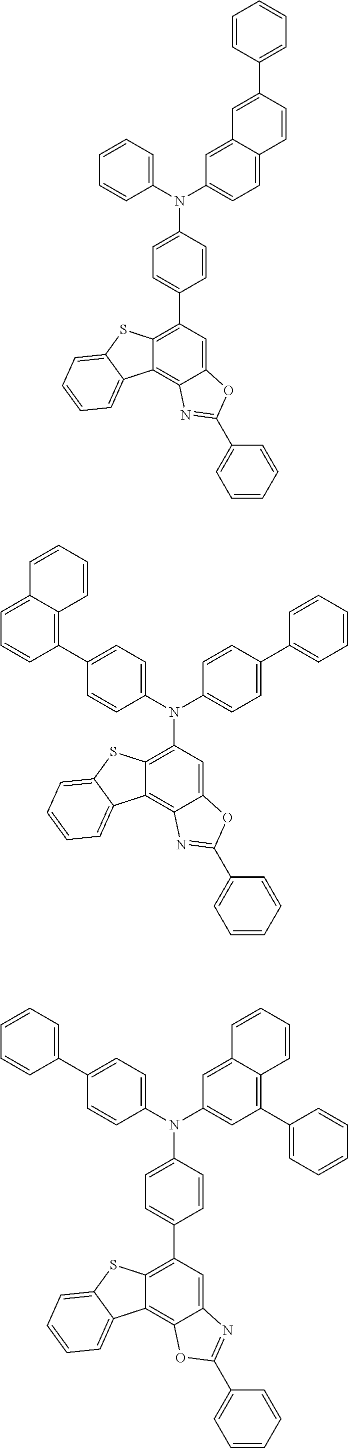 [SMILES: c1ccc(-c2ccc3ccc(N(c4ccccc4)c4ccc(-c5cc6oc(-c7ccccc7)nc6c6c5sc5ccccc56)cc4)cc3c2)cc1 ; c1ccc(-c2ccc(N(c3ccc(-c4cccc5ccccc45)cc3)c3cc4oc(-c5ccccc5)nc4c4c3sc3ccccc34)cc2)cc1 ; c1ccc(-c2ccc(N(c3ccc(-c4cc5nc(-c6ccccc6)oc5c5c4sc4ccccc45)cc3)c3cc(-c4ccccc4)c4ccccc4c3)cc2)cc1]

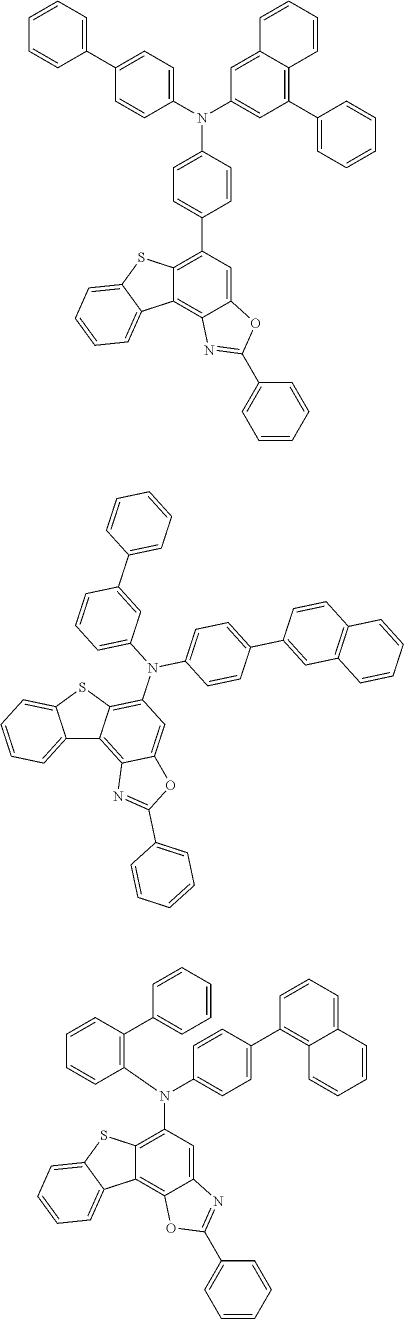 [SMILES: c1ccc(-c2ccc(N(c3ccc(-c4cc5oc(-c6ccccc6)nc5c5c4sc4ccccc45)cc3)c3cc(-c4ccccc4)c4ccccc4c3)cc2)cc1 ; c1ccc(-c2cccc(N(c3ccc(-c4ccc5ccccc5c4)cc3)c3cc4oc(-c5ccccc5)nc4c4c3sc3ccccc34)c2)cc1 ; c1ccc(-c2nc3cc(N(c4ccc(-c5cccc6ccccc56)cc4)c4ccccc4-c4ccccc4)c4sc5ccccc5c4c3o2)cc1]

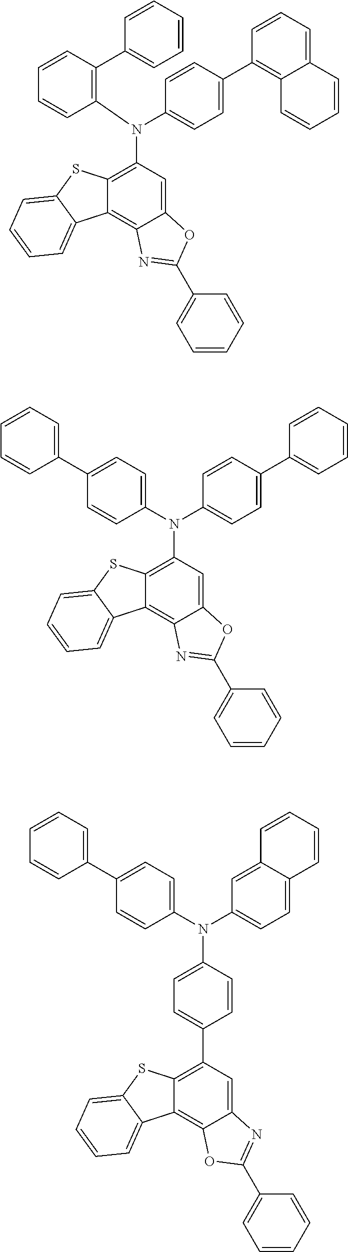 [SMILES: c1ccc(-c2nc3c(cc(N(c4ccc(-c5cccc6ccccc56)cc4)c4ccccc4-c4ccccc4)c4sc5ccccc5c43)o2)cc1 ; c1ccc(-c2ccc(N(c3ccc(-c4ccccc4)cc3)c3cc4oc(-c5ccccc5)nc4c4c3sc3ccccc34)cc2)cc1 ; c1ccc(-c2ccc(N(c3ccc(-c4cc5nc(-c6ccccc6)oc5c5c4sc4ccccc45)cc3)c3ccc4ccccc4c3)cc2)cc1]

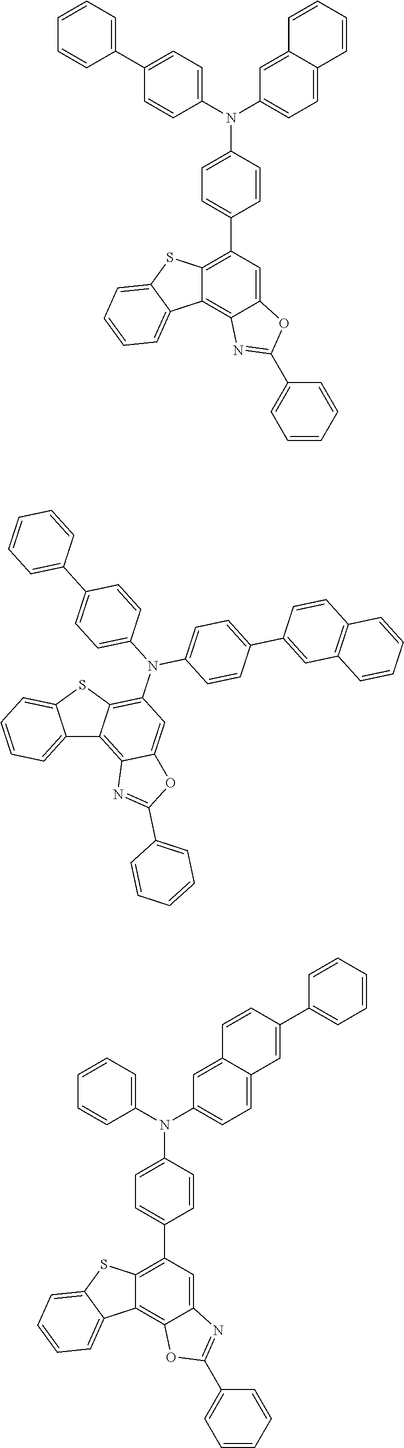 [SMILES: c1ccc(-c2ccc(N(c3ccc(-c4cc5oc(-c6ccccc6)nc5c5c4sc4ccccc45)cc3)c3ccc4ccccc4c3)cc2)cc1 ; c1ccc(-c2ccc(N(c3ccc(-c4ccc5ccccc5c4)cc3)c3cc4oc(-c5ccccc5)nc4c4c3sc3ccccc34)cc2)cc1 ; c1ccc(-c2ccc3cc(N(c4ccccc4)c4ccc(-c5cc6nc(-c7ccccc7)oc6c6c5sc5ccccc56)cc4)ccc3c2)cc1]

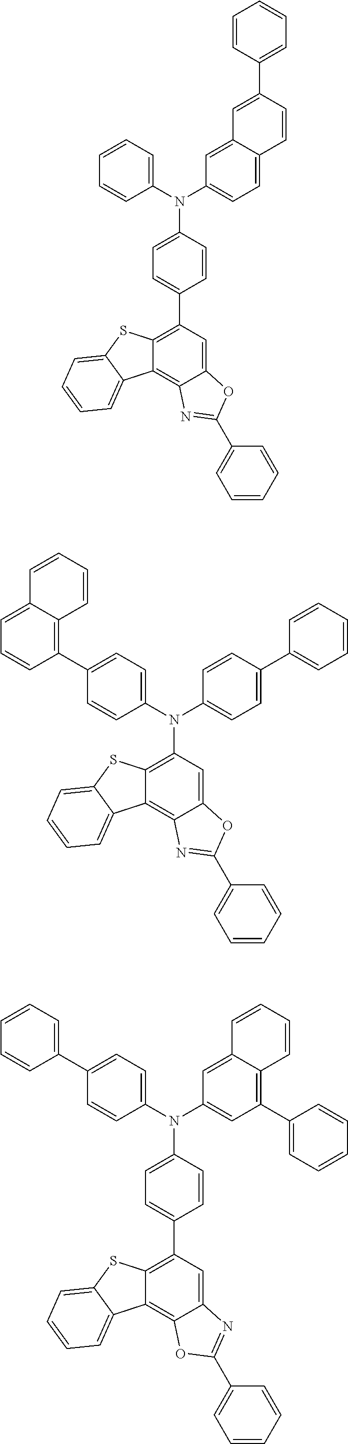 [SMILES: c1ccc(-c2ccc3ccc(N(c4ccccc4)c4ccc(-c5cc6oc(-c7ccccc7)nc6c6c5sc5ccccc56)cc4)cc3c2)cc1 ; c1ccc(-c2ccc(N(c3ccc(-c4cccc5ccccc45)cc3)c3cc4oc(-c5ccccc5)nc4c4c3sc3ccccc34)cc2)cc1 ; c1ccc(-c2ccc(N(c3ccc(-c4cc5nc(-c6ccccc6)oc5c5c4sc4ccccc45)cc3)c3cc(-c4ccccc4)c4ccccc4c3)cc2)cc1]

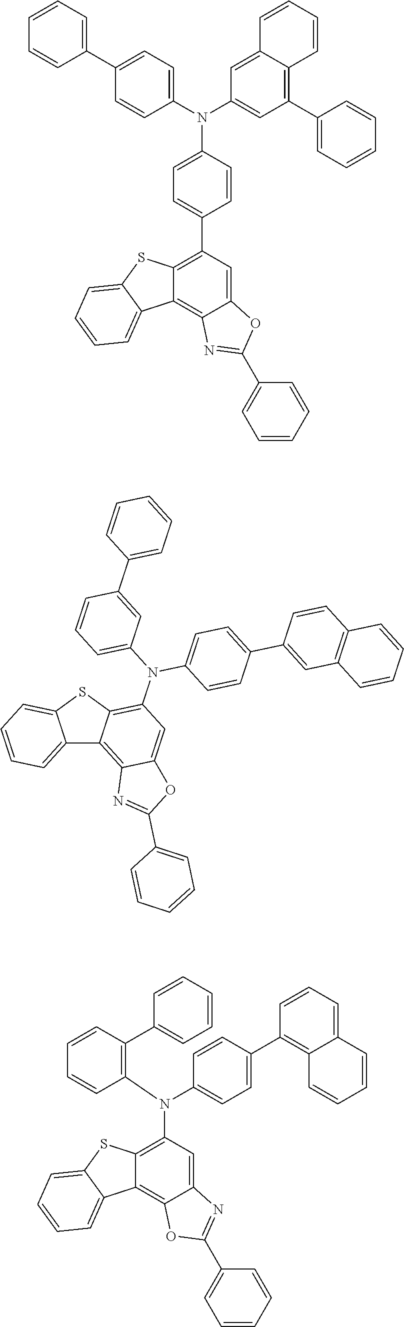 [SMILES: c1ccc(-c2ccc(N(c3ccc(-c4cc5oc(-c6ccccc6)nc5c5c4sc4ccccc45)cc3)c3cc(-c4ccccc4)c4ccccc4c3)cc2)cc1 ; c1ccc(-c2cccc(N(c3ccc(-c4ccc5ccccc5c4)cc3)c3cc4oc(-c5ccccc5)nc4c4c3sc3ccccc34)c2)cc1 ; c1ccc(-c2nc3cc(N(c4ccc(-c5cccc6ccccc56)cc4)c4ccccc4-c4ccccc4)c4sc5ccccc5c4c3o2)cc1]

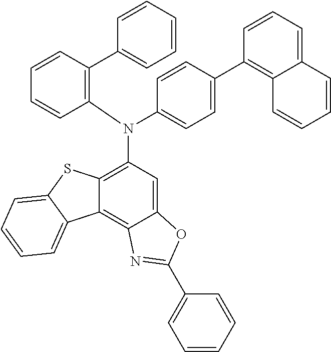 [SMILES: c1ccc(-c2nc3c(cc(N(c4ccc(-c5cccc6ccccc56)cc4)c4ccccc4-c4ccccc4)c4sc5ccccc5c43)o2)cc1]

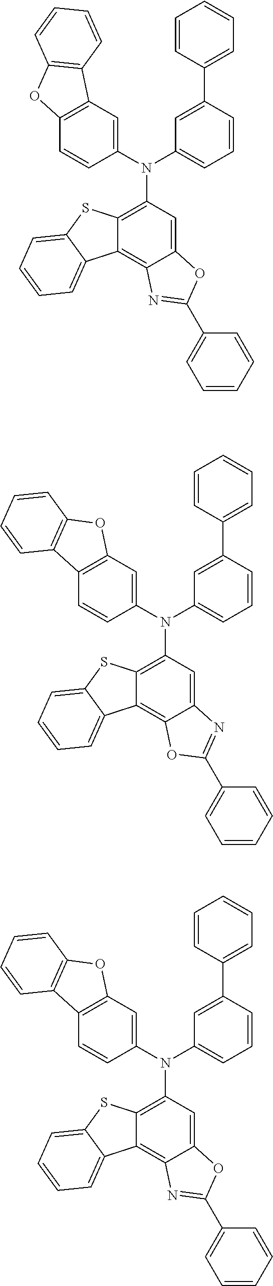 [SMILES: c1ccc(-c2cccc(N(c3ccc4oc5ccccc5c4c3)c3cc4oc(-c5ccccc5)nc4c4c3sc3ccccc34)c2)cc1 ; c1ccc(-c2cccc(N(c3ccc4c(c3)oc3ccccc34)c3cc4nc(-c5ccccc5)oc4c4c3sc3ccccc34)c2)cc1 ; c1ccc(-c2cccc(N(c3ccc4c(c3)oc3ccccc34)c3cc4oc(-c5ccccc5)nc4c4c3sc3ccccc34)c2)cc1]

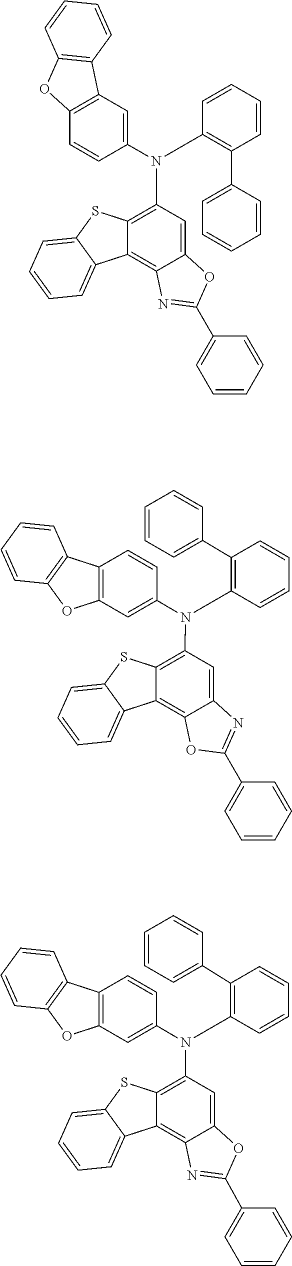 [SMILES: c1ccc(-c2nc3c(cc(N(c4ccc5oc6ccccc6c5c4)c4ccccc4-c4ccccc4)c4sc5ccccc5c43)o2)cc1 ; c1ccc(-c2nc3cc(N(c4ccc5c(c4)oc4ccccc45)c4ccccc4-c4ccccc4)c4sc5ccccc5c4c3o2)cc1 ; c1ccc(-c2nc3c(cc(N(c4ccc5c(c4)oc4ccccc45)c4ccccc4-c4ccccc4)c4sc5ccccc5c43)o2)cc1]

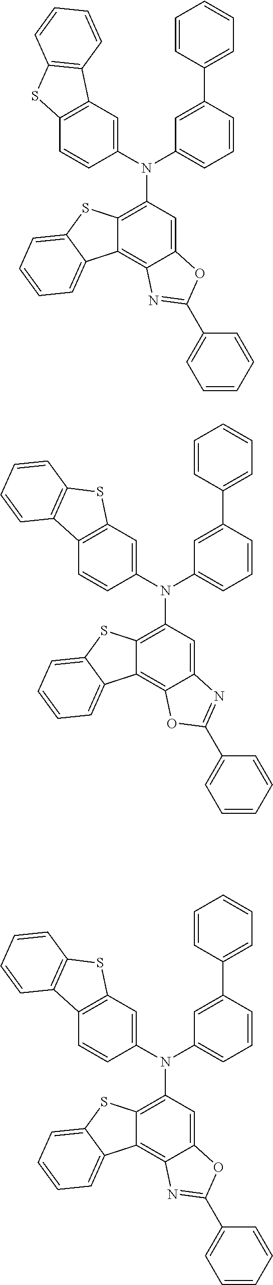

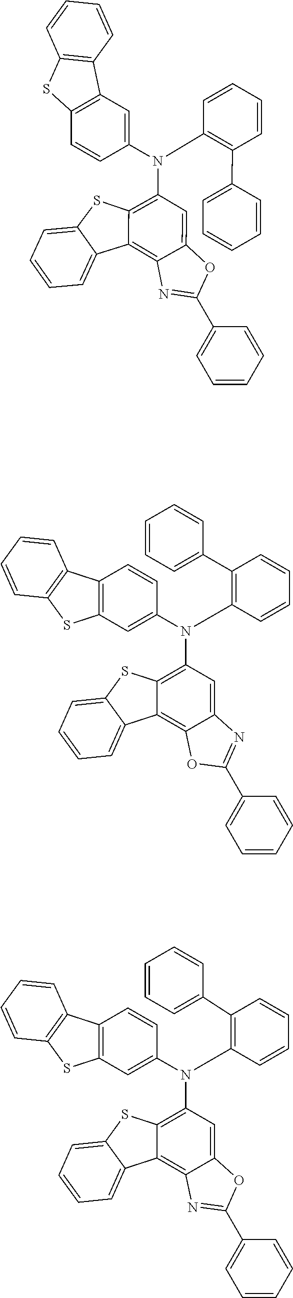 [SMILES: c1ccc(-c2nc3c(cc(N(c4ccc5sc6ccccc6c5c4)c4ccccc4-c4ccccc4)c4sc5ccccc5c43)o2)cc1 ; c1ccc(-c2nc3cc(N(c4ccc5c(c4)sc4ccccc45)c4ccccc4-c4ccccc4)c4sc5ccccc5c4c3o2)cc1 ; c1ccc(-c2nc3c(cc(N(c4ccc5c(c4)sc4ccccc45)c4ccccc4-c4ccccc4)c4sc5ccccc5c43)o2)cc1]

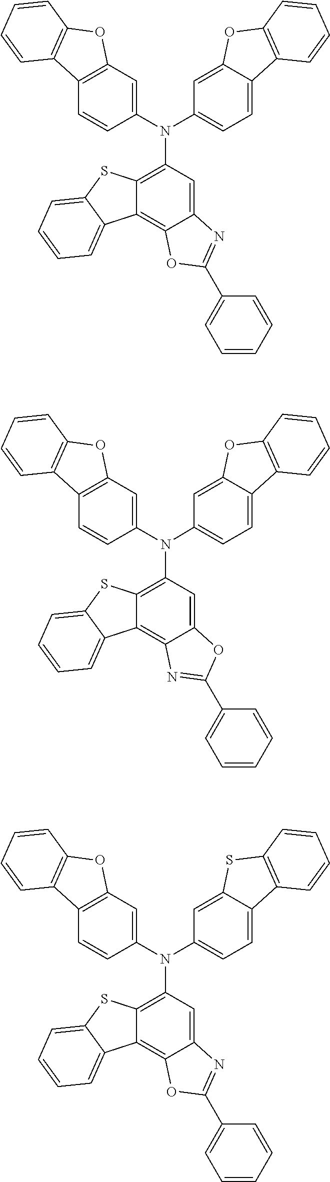 [SMILES: c1ccc(-c2nc3cc(N(c4ccc5c(c4)oc4ccccc45)c4ccc5c(c4)oc4ccccc45)c4sc5ccccc5c4c3o2)cc1 ; c1ccc(-c2nc3c(cc(N(c4ccc5c(c4)oc4ccccc45)c4ccc5c(c4)oc4ccccc45)c4sc5ccccc5c43)o2)cc1 ; c1ccc(-c2nc3cc(N(c4ccc5c(c4)oc4ccccc45)c4ccc5c(c4)sc4ccccc45)c4sc5ccccc5c4c3o2)cc1]

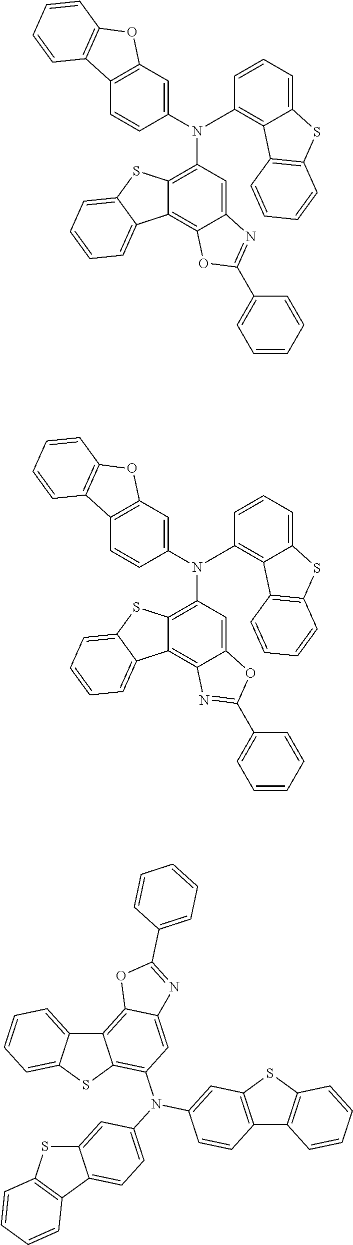 [SMILES: c1ccc(-c2nc3cc(N(c4ccc5c(c4)oc4ccccc45)c4cccc5sc6ccccc6c45)c4sc5ccccc5c4c3o2)cc1 ; c1ccc(-c2nc3c(cc(N(c4ccc5c(c4)oc4ccccc45)c4cccc5sc6ccccc6c45)c4sc5ccccc5c43)o2)cc1 ; c1ccc(-c2nc3cc(N(c4ccc5c(c4)sc4ccccc45)c4ccc5c(c4)sc4ccccc45)c4sc5ccccc5c4c3o2)cc1]

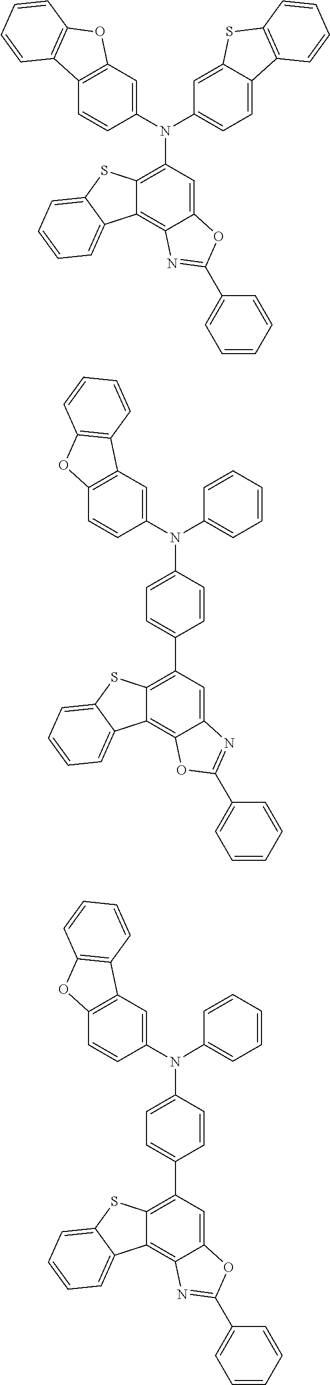 [SMILES: c1ccc(-c2nc3c(cc(N(c4ccc5c(c4)oc4ccccc45)c4ccc5c(c4)sc4ccccc45)c4sc5ccccc5c43)o2)cc1 ; c1ccc(-c2nc3cc(-c4ccc(N(c5ccccc5)c5ccc6oc7ccccc7c6c5)cc4)c4sc5ccccc5c4c3o2)cc1 ; c1ccc(-c2nc3c(cc(-c4ccc(N(c5ccccc5)c5ccc6oc7ccccc7c6c5)cc4)c4sc5ccccc5c43)o2)cc1]

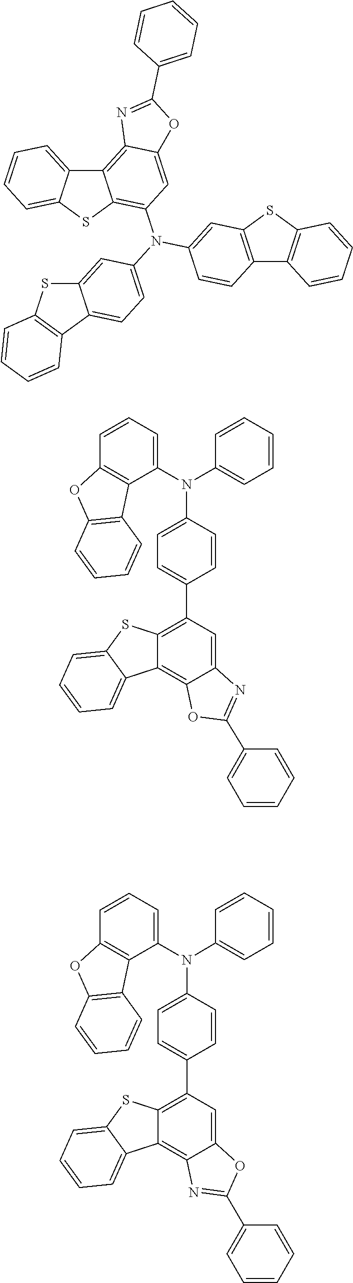 [SMILES: c1ccc(-c2nc3c(cc(N(c4ccc5c(c4)sc4ccccc45)c4ccc5c(c4)sc4ccccc45)c4sc5ccccc5c43)o2)cc1 ; c1ccc(-c2nc3cc(-c4ccc(N(c5ccccc5)c5cccc6oc7ccccc7c56)cc4)c4sc5ccccc5c4c3o2)cc1 ; c1ccc(-c2nc3c(cc(-c4ccc(N(c5ccccc5)c5cccc6oc7ccccc7c56)cc4)c4sc5ccccc5c43)o2)cc1]

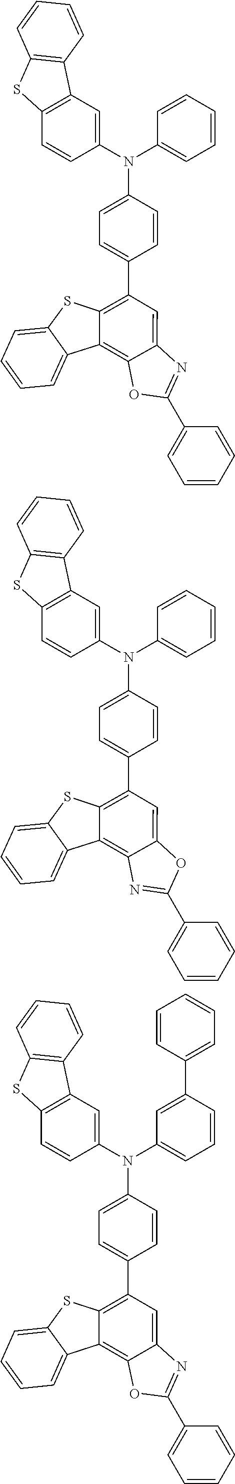

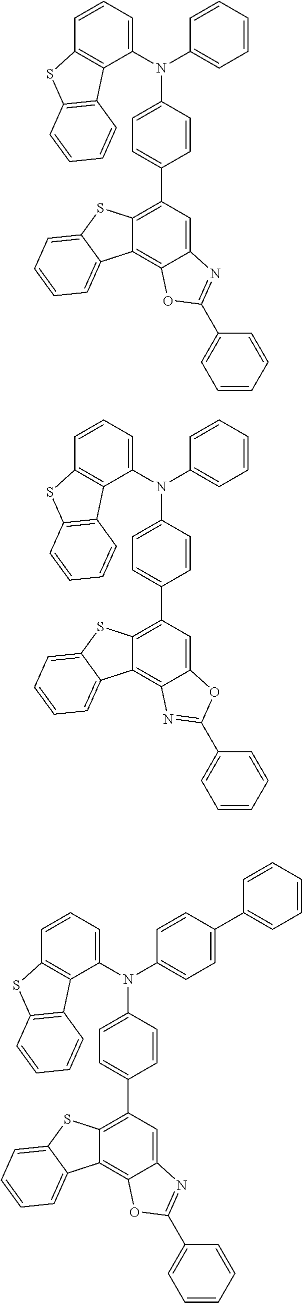

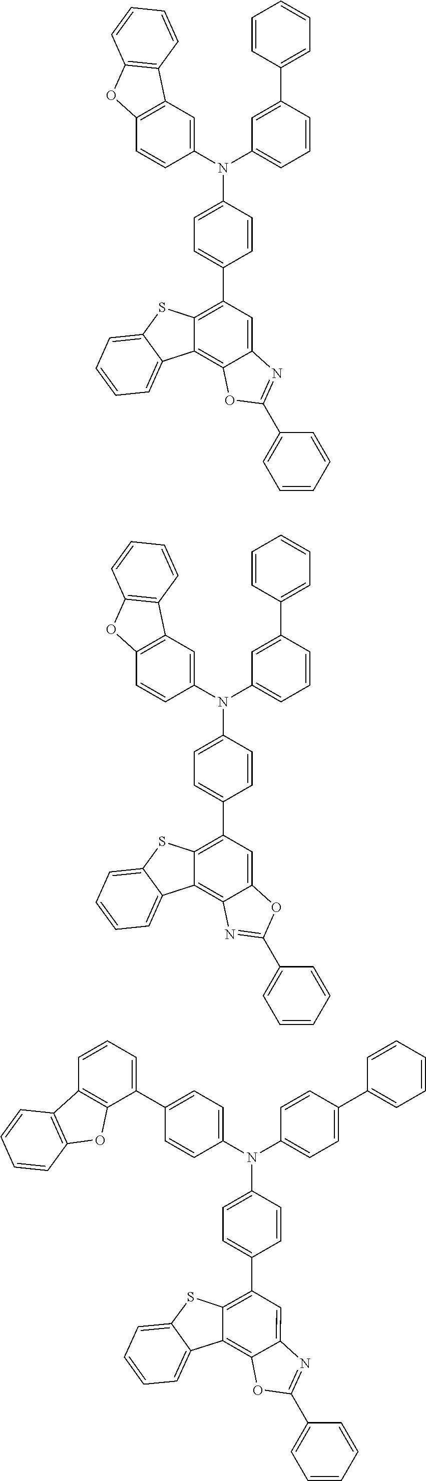

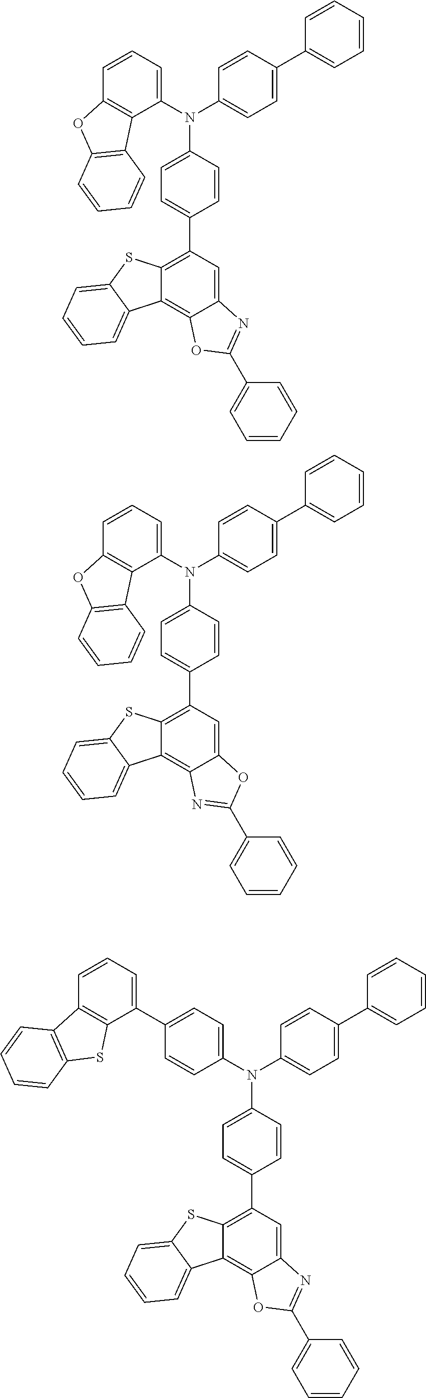

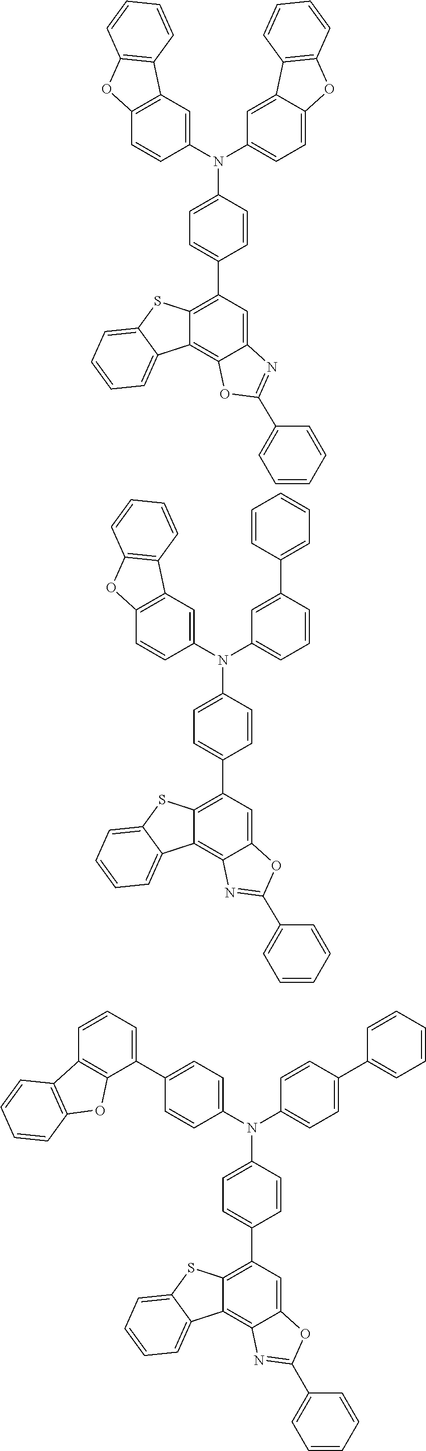

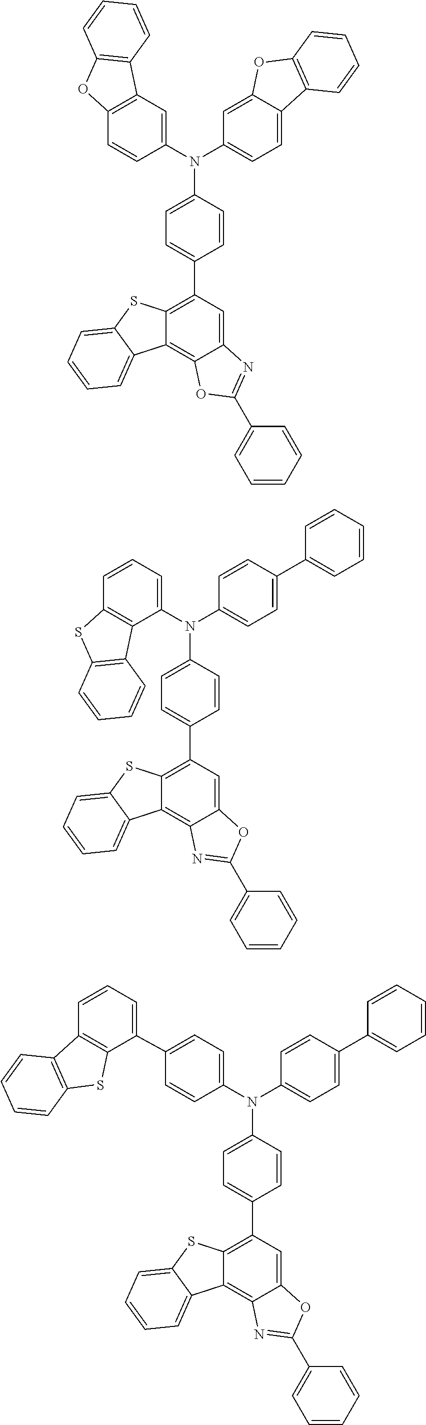 [SMILES: c1ccc(-c2nc3cc(-c4ccc(N(c5ccc6c(c5)oc5ccccc56)c5ccc6oc7ccccc7c6c5)cc4)c4sc5ccccc5c4c3o2)cc1 ; c1ccc(-c2ccc(N(c3ccc(-c4cc5oc(-c6ccccc6)nc5c5c4sc4ccccc45)cc3)c3cccc4sc5ccccc5c34)cc2)cc1 ; c1ccc(-c2ccc(N(c3ccc(-c4cccc5c4sc4ccccc45)cc3)c3ccc(-c4cc5oc(-c6ccccc6)nc5c5c4sc4ccccc45)cc3)cc2)cc1]

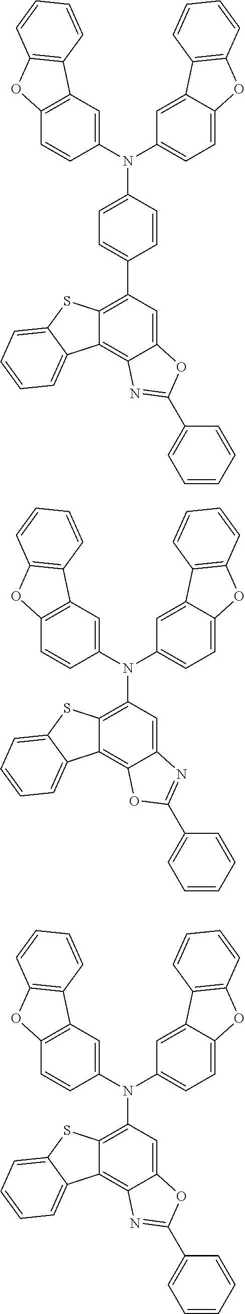

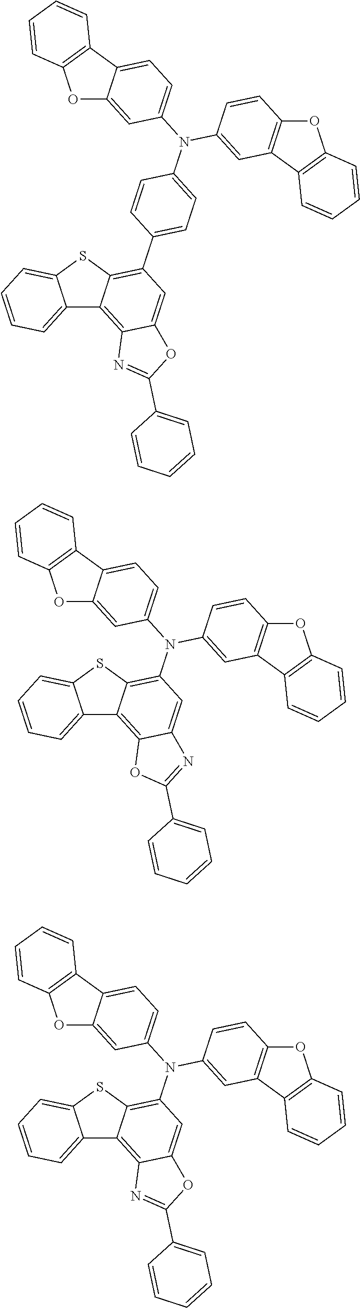 [SMILES: c1ccc(-c2nc3c(cc(-c4ccc(N(c5ccc6c(c5)oc5ccccc56)c5ccc6oc7ccccc7c6c5)cc4)c4sc5ccccc5c43)o2)cc1 ; c1ccc(-c2nc3cc(N(c4ccc5c(c4)oc4ccccc45)c4ccc5oc6ccccc6c5c4)c4sc5ccccc5c4c3o2)cc1 ; c1ccc(-c2nc3c(cc(N(c4ccc5c(c4)oc4ccccc45)c4ccc5oc6ccccc6c5c4)c4sc5ccccc5c43)o2)cc1]

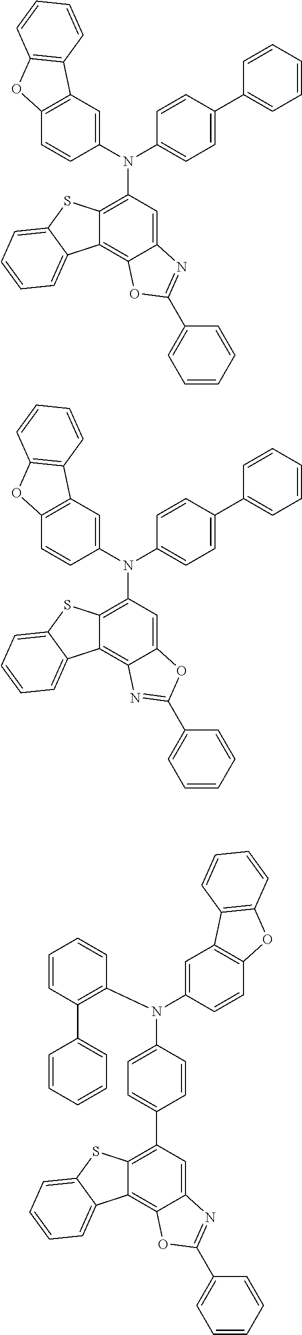 [SMILES: c1ccc(-c2ccc(N(c3ccc4oc5ccccc5c4c3)c3cc4nc(-c5ccccc5)oc4c4c3sc3ccccc34)cc2)cc1 ; c1ccc(-c2ccc(N(c3ccc4oc5ccccc5c4c3)c3cc4oc(-c5ccccc5)nc4c4c3sc3ccccc34)cc2)cc1 ; c1ccc(-c2nc3cc(-c4ccc(N(c5ccc6oc7ccccc7c6c5)c5ccccc5-c5ccccc5)cc4)c4sc5ccccc5c4c3o2)cc1]

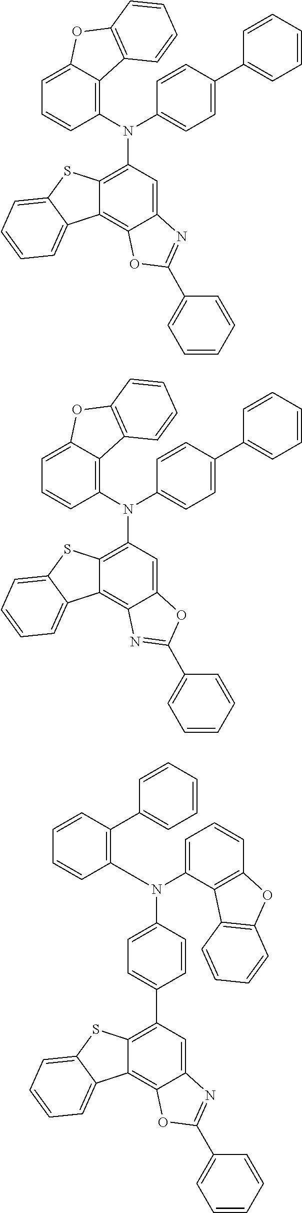

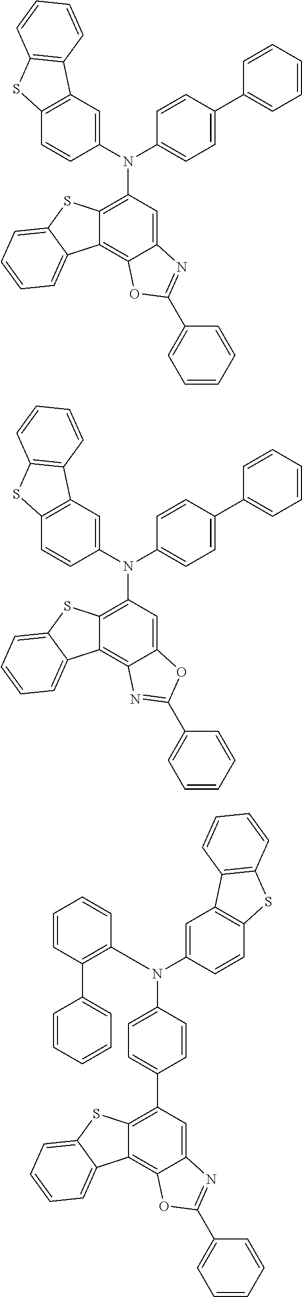

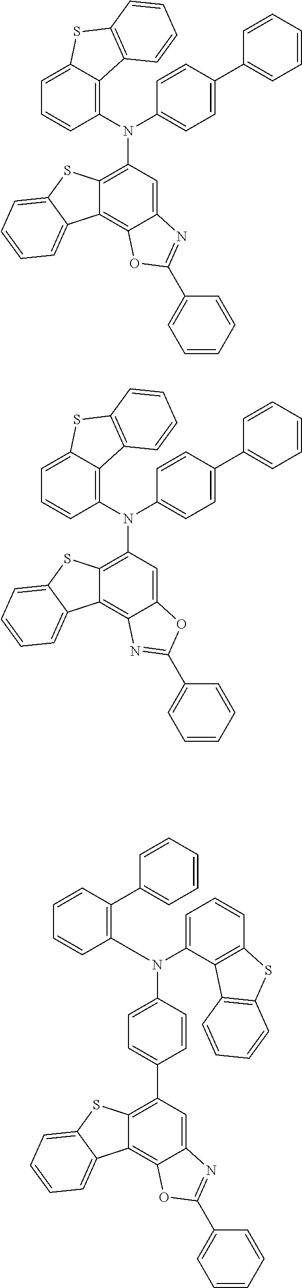

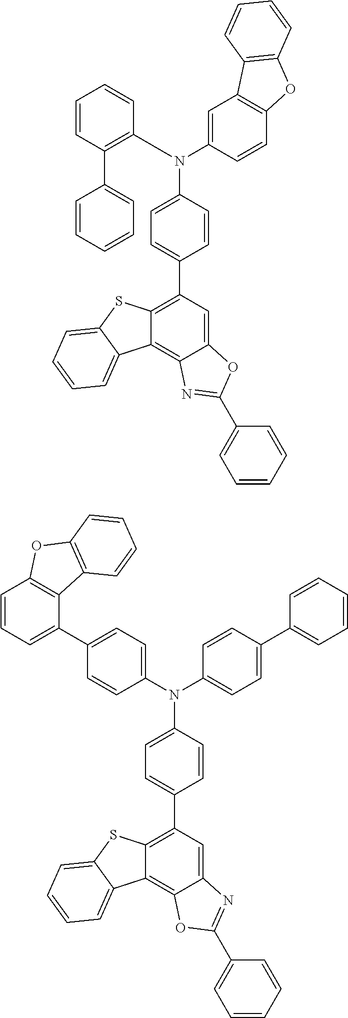 [SMILES: c1ccc(-c2nc3c(cc(-c4ccc(N(c5ccc6oc7ccccc7c6c5)c5ccccc5-c5ccccc5)cc4)c4sc5ccccc5c43)o2)cc1 ; c1ccc(-c2ccc(N(c3ccc(-c4cc5nc(-c6ccccc6)oc5c5c4sc4ccccc45)cc3)c3ccc(-c4cccc5oc6ccccc6c45)cc3)cc2)cc1]

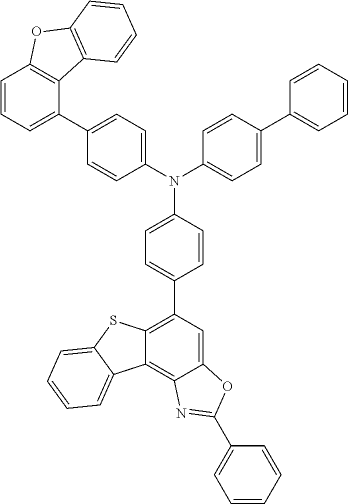 [SMILES: c1ccc(-c2ccc(N(c3ccc(-c4cc5oc(-c6ccccc6)nc5c5c4sc4ccccc45)cc3)c3ccc(-c4cccc5oc6ccccc6c45)cc3)cc2)cc1]

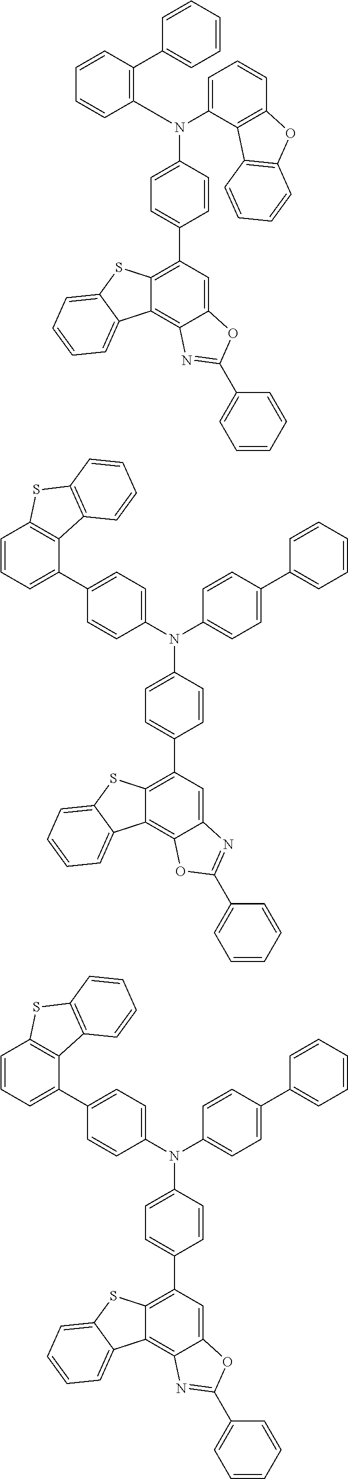 [SMILES: c1ccc(-c2nc3c(cc(-c4ccc(N(c5ccccc5-c5ccccc5)c5cccc6oc7ccccc7c56)cc4)c4sc5ccccc5c43)o2)cc1 ; c1ccc(-c2ccc(N(c3ccc(-c4cc5nc(-c6ccccc6)oc5c5c4sc4ccccc45)cc3)c3ccc(-c4cccc5sc6ccccc6c45)cc3)cc2)cc1 ; c1ccc(-c2ccc(N(c3ccc(-c4cc5oc(-c6ccccc6)nc5c5c4sc4ccccc45)cc3)c3ccc(-c4cccc5sc6ccccc6c45)cc3)cc2)cc1]

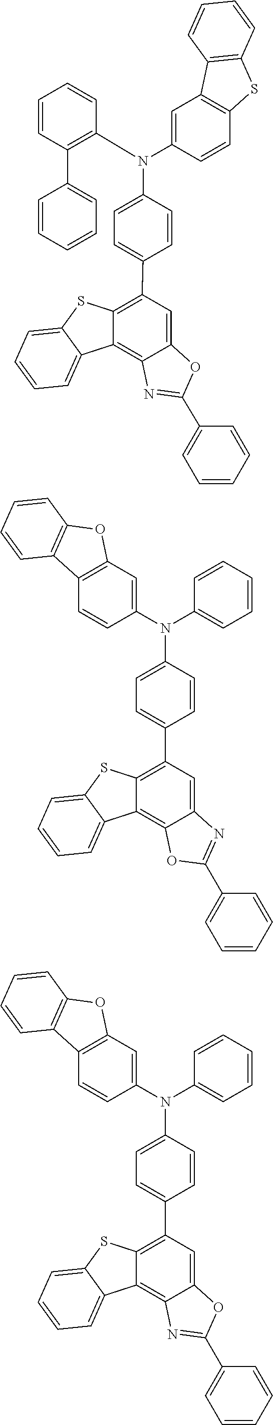 [SMILES: c1ccc(-c2nc3c(cc(-c4ccc(N(c5ccc6sc7ccccc7c6c5)c5ccccc5-c5ccccc5)cc4)c4sc5ccccc5c43)o2)cc1 ; c1ccc(-c2nc3cc(-c4ccc(N(c5ccccc5)c5ccc6c(c5)oc5ccccc56)cc4)c4sc5ccccc5c4c3o2)cc1 ; c1ccc(-c2nc3c(cc(-c4ccc(N(c5ccccc5)c5ccc6c(c5)oc5ccccc56)cc4)c4sc5ccccc5c43)o2)cc1]

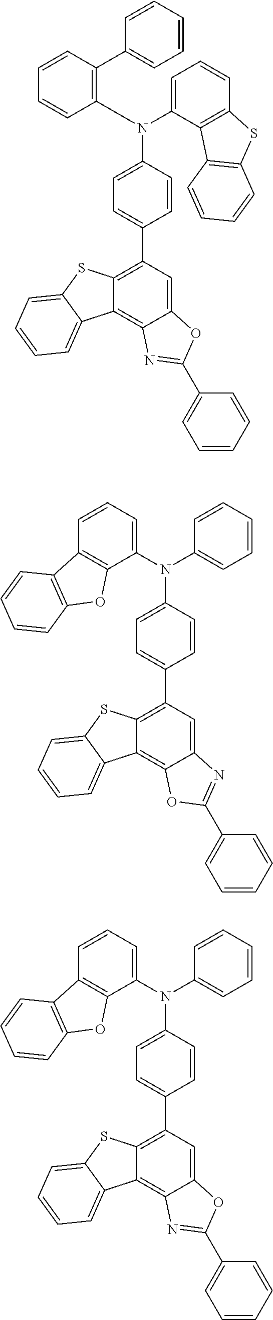

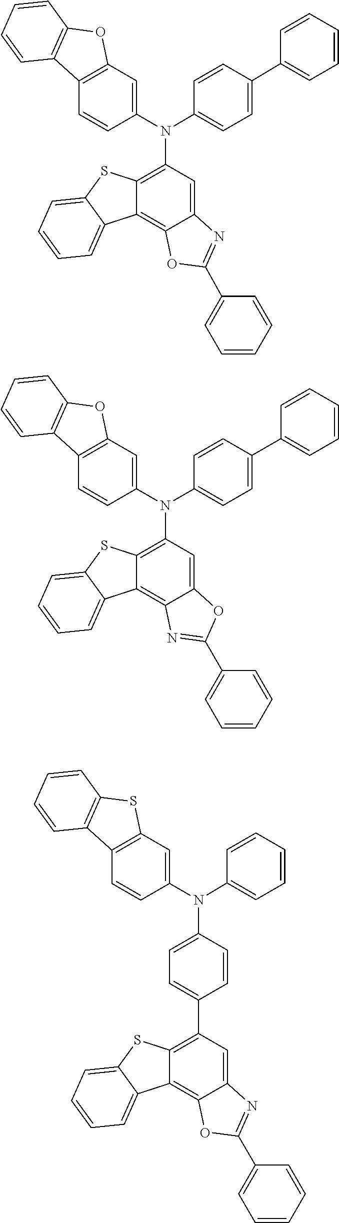 [SMILES: c1ccc(-c2ccc(N(c3ccc4c(c3)oc3ccccc34)c3cc4nc(-c5ccccc5)oc4c4c3sc3ccccc34)cc2)cc1 ; c1ccc(-c2ccc(N(c3ccc4c(c3)oc3ccccc34)c3cc4oc(-c5ccccc5)nc4c4c3sc3ccccc34)cc2)cc1 ; c1ccc(-c2nc3cc(-c4ccc(N(c5ccccc5)c5ccc6c(c5)sc5ccccc56)cc4)c4sc5ccccc5c4c3o2)cc1]

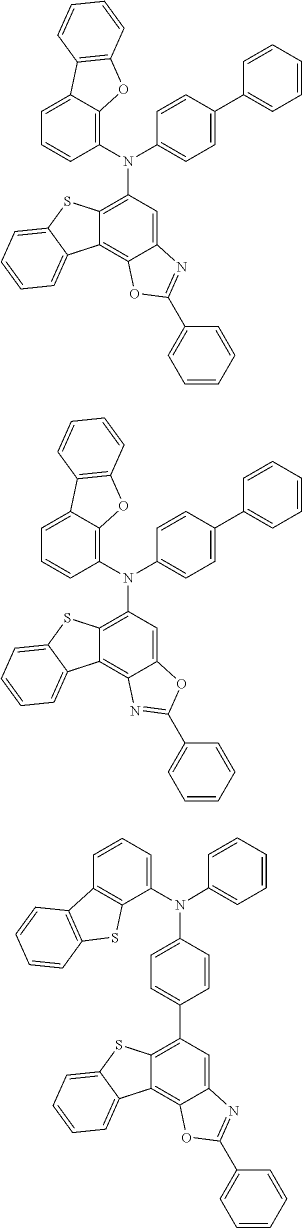

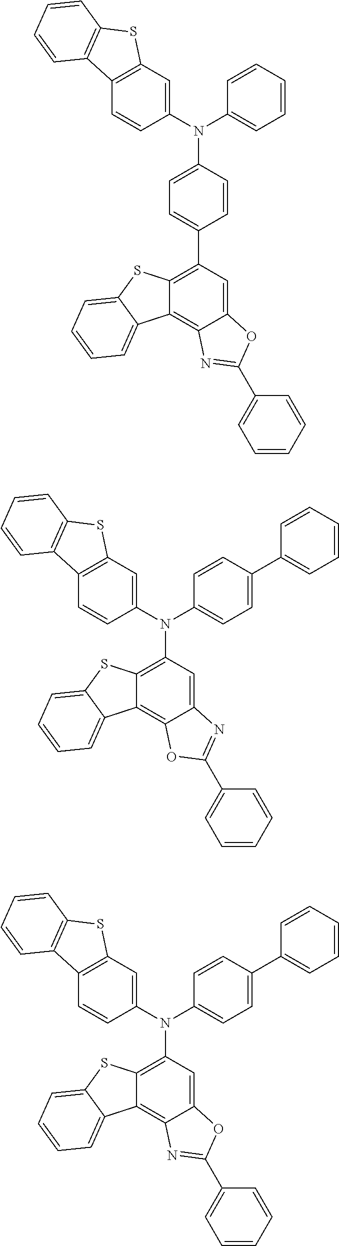 [SMILES: c1ccc(-c2nc3c(cc(-c4ccc(N(c5ccccc5)c5ccc6c(c5)sc5ccccc56)cc4)c4sc5ccccc5c43)o2)cc1 ; c1ccc(-c2ccc(N(c3ccc4c(c3)sc3ccccc34)c3cc4nc(-c5ccccc5)oc4c4c3sc3ccccc34)cc2)cc1 ; c1ccc(-c2ccc(N(c3ccc4c(c3)sc3ccccc34)c3cc4oc(-c5ccccc5)nc4c4c3sc3ccccc34)cc2)cc1]

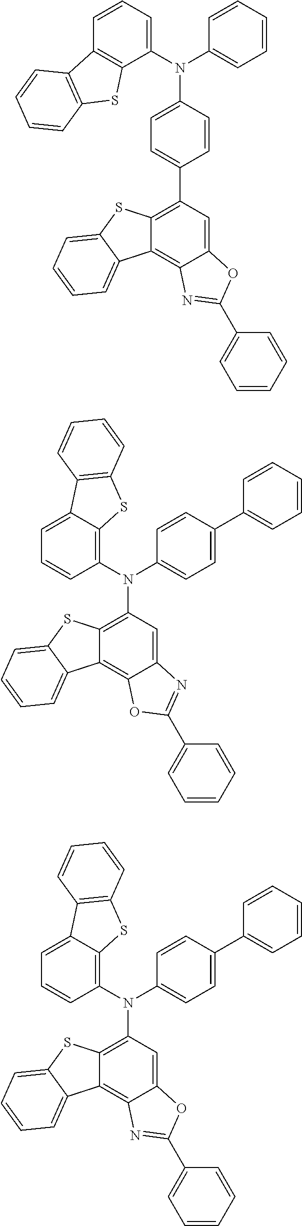 [SMILES: c1ccc(-c2nc3c(cc(-c4ccc(N(c5ccccc5)c5cccc6c5sc5ccccc56)cc4)c4sc5ccccc5c43)o2)cc1 ; c1ccc(-c2ccc(N(c3cccc4c3sc3ccccc34)c3cc4nc(-c5ccccc5)oc4c4c3sc3ccccc34)cc2)cc1 ; c1ccc(-c2ccc(N(c3cccc4c3sc3ccccc34)c3cc4oc(-c5ccccc5)nc4c4c3sc3ccccc34)cc2)cc1]

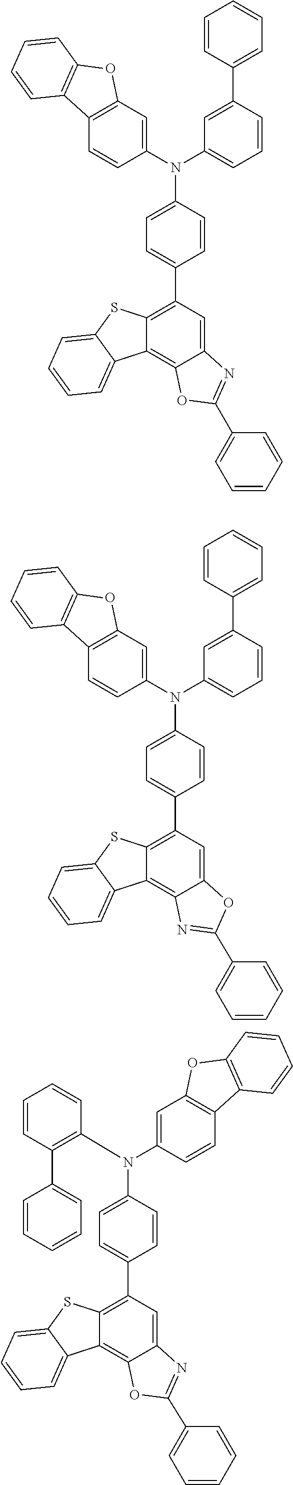

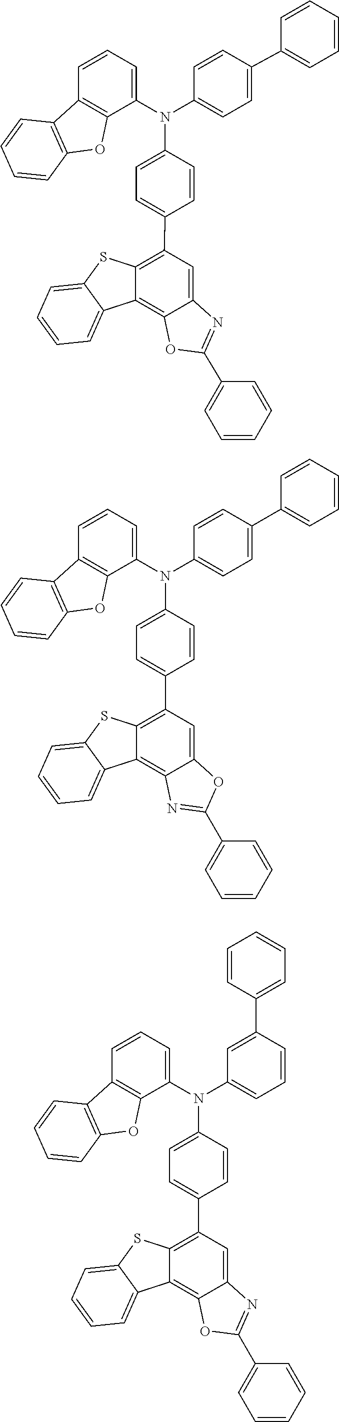

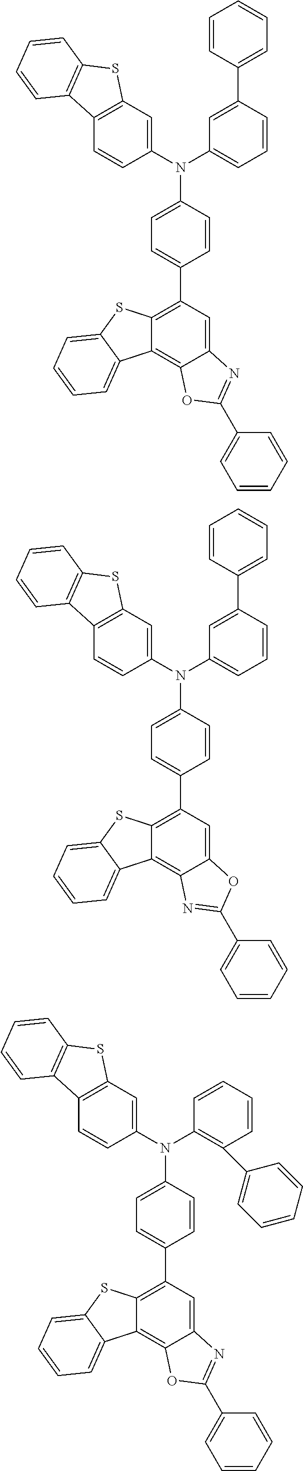

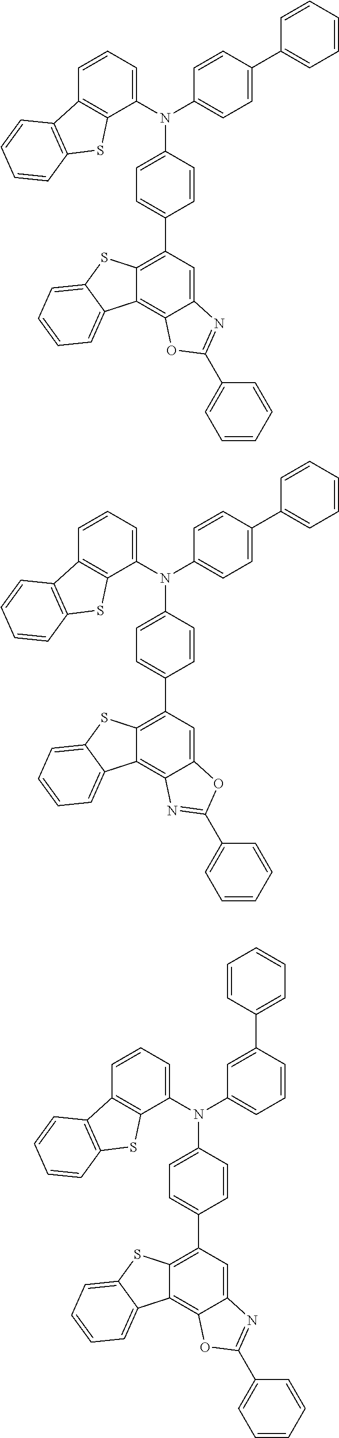

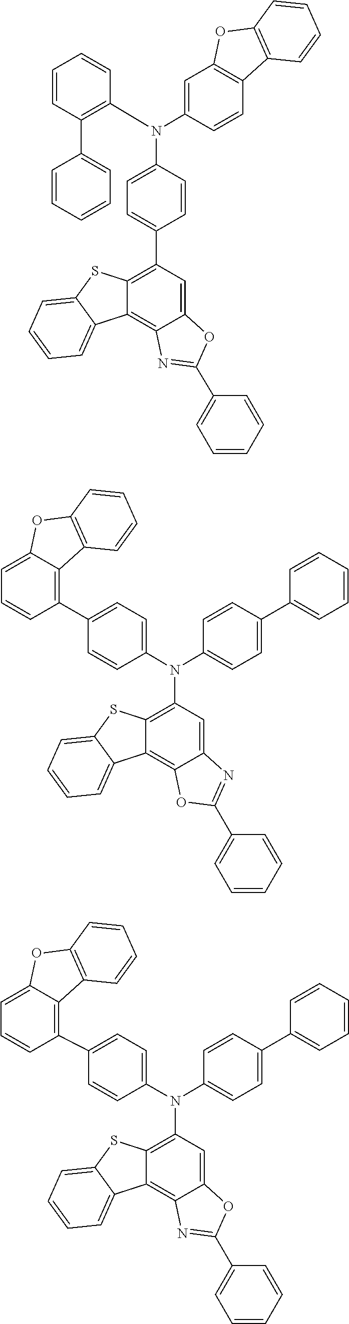 [SMILES: c1ccc(-c2nc3c(cc(-c4ccc(N(c5ccc6c(c5)oc5ccccc56)c5ccccc5-c5ccccc5)cc4)c4sc5ccccc5c43)o2)cc1 ; c1ccc(-c2ccc(N(c3ccc(-c4cccc5oc6ccccc6c45)cc3)c3cc4nc(-c5ccccc5)oc4c4c3sc3ccccc34)cc2)cc1 ; c1ccc(-c2ccc(N(c3ccc(-c4cccc5oc6ccccc6c45)cc3)c3cc4oc(-c5ccccc5)nc4c4c3sc3ccccc34)cc2)cc1]

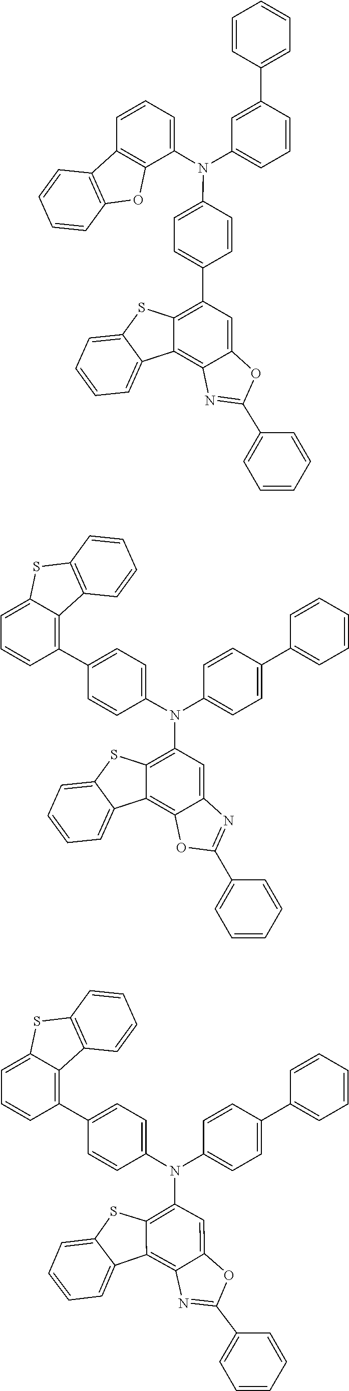 [SMILES: c1ccc(-c2cccc(N(c3ccc(-c4cc5oc(-c6ccccc6)nc5c5c4sc4ccccc45)cc3)c3cccc4c3oc3ccccc34)c2)cc1 ; c1ccc(-c2ccc(N(c3ccc(-c4cccc5sc6ccccc6c45)cc3)c3cc4nc(-c5ccccc5)oc4c4c3sc3ccccc34)cc2)cc1 ; c1ccc(-c2ccc(N(c3ccc(-c4cccc5sc6ccccc6c45)cc3)c3cc4oc(-c5ccccc5)nc4c4c3sc3ccccc34)cc2)cc1]

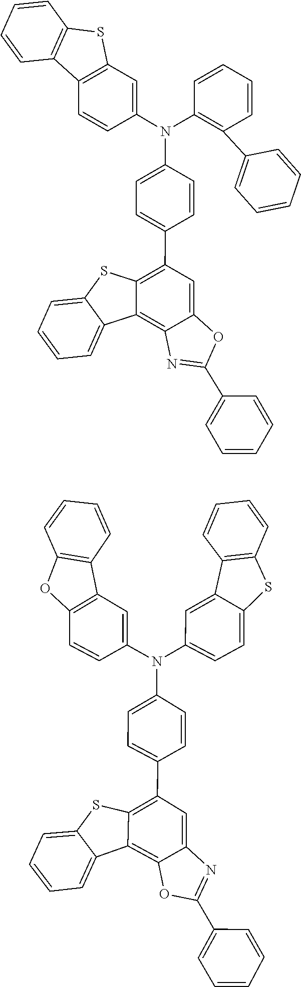 [SMILES: c1ccc(-c2nc3c(cc(-c4ccc(N(c5ccc6c(c5)sc5ccccc56)c5ccccc5-c5ccccc5)cc4)c4sc5ccccc5c43)o2)cc1 ; c1ccc(-c2nc3cc(-c4ccc(N(c5ccc6oc7ccccc7c6c5)c5ccc6sc7ccccc7c6c5)cc4)c4sc5ccccc5c4c3o2)cc1]

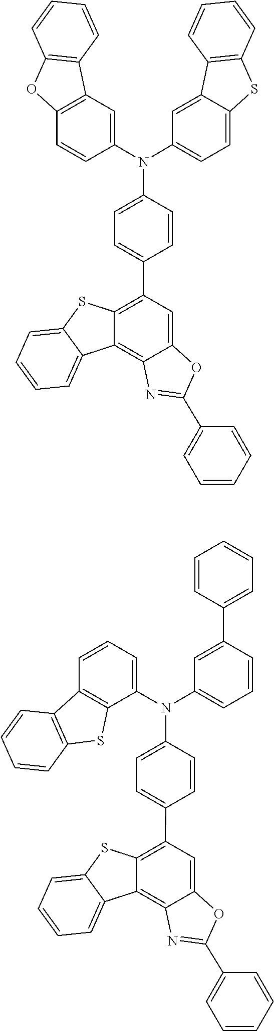 [SMILES: c1ccc(-c2nc3c(cc(-c4ccc(N(c5ccc6oc7ccccc7c6c5)c5ccc6sc7ccccc7c6c5)cc4)c4sc5ccccc5c43)o2)cc1 ; c1ccc(-c2cccc(N(c3ccc(-c4cc5oc(-c6ccccc6)nc5c5c4sc4ccccc45)cc3)c3cccc4c3sc3ccccc34)c2)cc1]

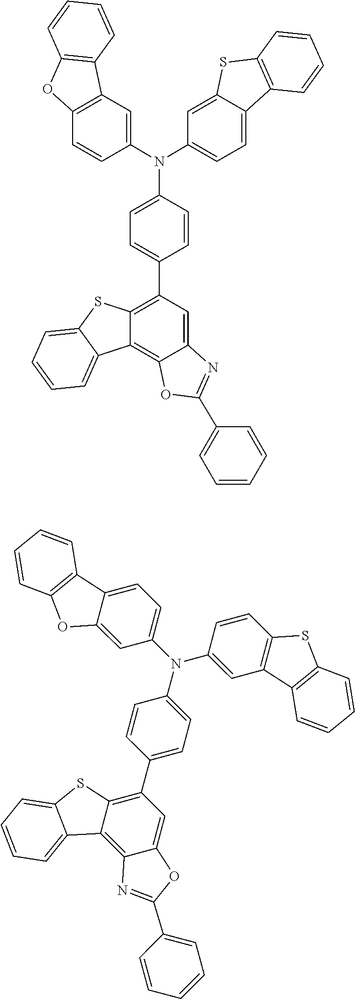 [SMILES: c1ccc(-c2nc3cc(-c4ccc(N(c5ccc6c(c5)sc5ccccc56)c5ccc6oc7ccccc7c6c5)cc4)c4sc5ccccc5c4c3o2)cc1 ; c1ccc(-c2nc3c(cc(-c4ccc(N(c5ccc6c(c5)oc5ccccc56)c5ccc6sc7ccccc7c6c5)cc4)c4sc5ccccc5c43)o2)cc1]

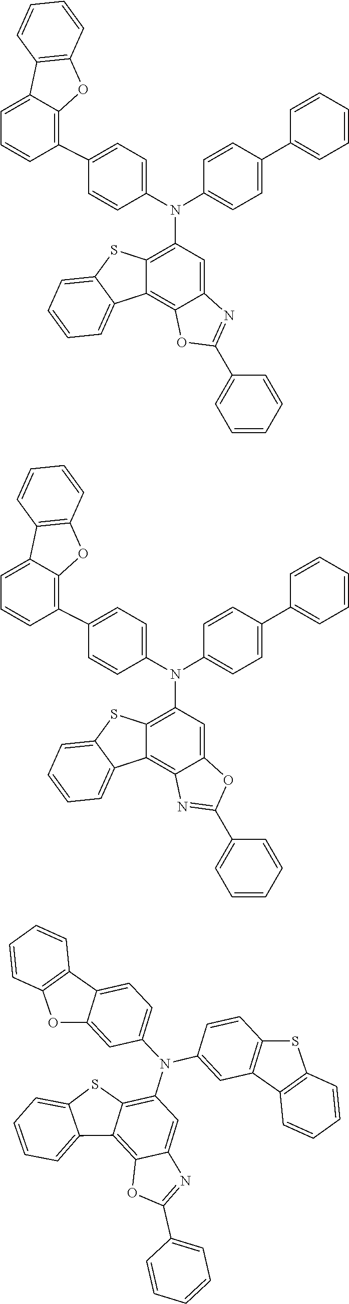 [SMILES: c1ccc(-c2ccc(N(c3ccc(-c4cccc5c4oc4ccccc45)cc3)c3cc4nc(-c5ccccc5)oc4c4c3sc3ccccc34)cc2)cc1 ; c1ccc(-c2ccc(N(c3ccc(-c4cccc5c4oc4ccccc45)cc3)c3cc4oc(-c5ccccc5)nc4c4c3sc3ccccc34)cc2)cc1 ; c1ccc(-c2nc3cc(N(c4ccc5c(c4)oc4ccccc45)c4ccc5sc6ccccc6c5c4)c4sc5ccccc5c4c3o2)cc1]

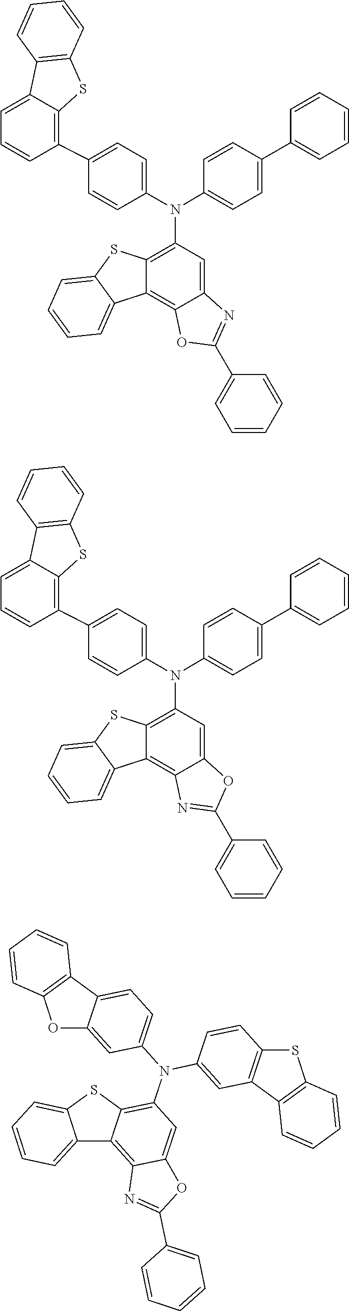 [SMILES: c1ccc(-c2ccc(N(c3ccc(-c4cccc5c4sc4ccccc45)cc3)c3cc4nc(-c5ccccc5)oc4c4c3sc3ccccc34)cc2)cc1 ; c1ccc(-c2ccc(N(c3ccc(-c4cccc5c4sc4ccccc45)cc3)c3cc4oc(-c5ccccc5)nc4c4c3sc3ccccc34)cc2)cc1 ; c1ccc(-c2nc3c(cc(N(c4ccc5c(c4)oc4ccccc45)c4ccc5sc6ccccc6c5c4)c4sc5ccccc5c43)o2)cc1]

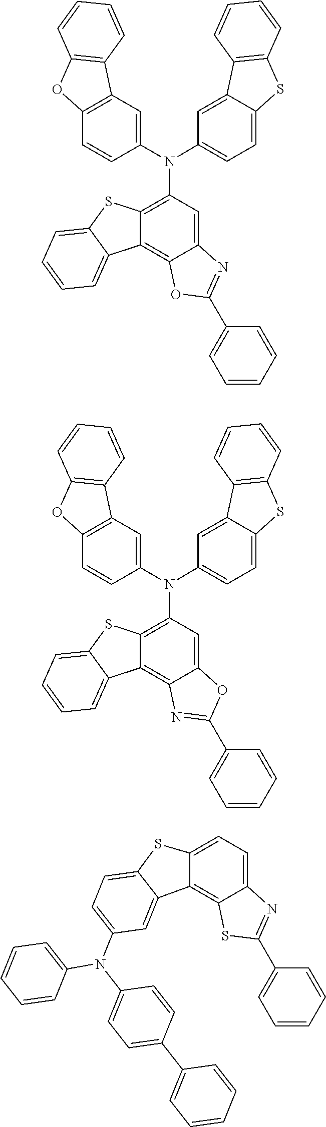

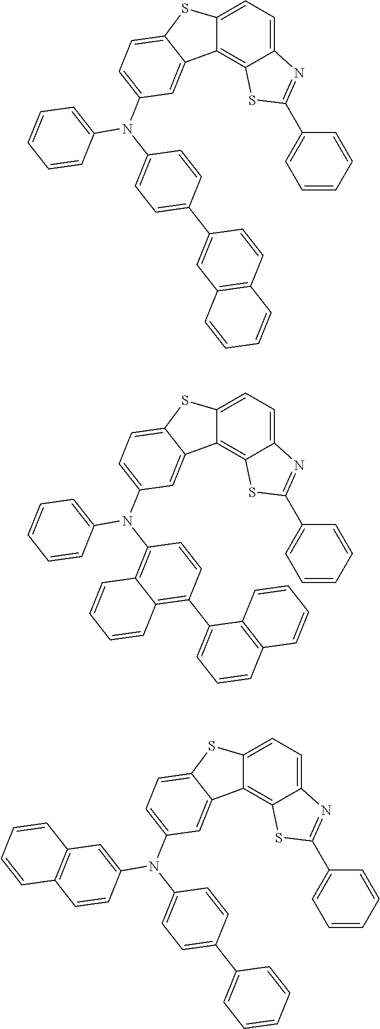 [SMILES: c1ccc(-c2nc3ccc4sc5ccc(N(c6ccccc6)c6ccc(-c7ccc8ccccc8c7)cc6)cc5c4c3s2)cc1 ; c1ccc(-c2nc3ccc4sc5ccc(N(c6ccccc6)c6ccc(-c7cccc8ccccc78)c7ccccc67)cc5c4c3s2)cc1 ; c1ccc(-c2ccc(N(c3ccc4ccccc4c3)c3ccc4sc5ccc6nc(-c7ccccc7)sc6c5c4c3)cc2)cc1]

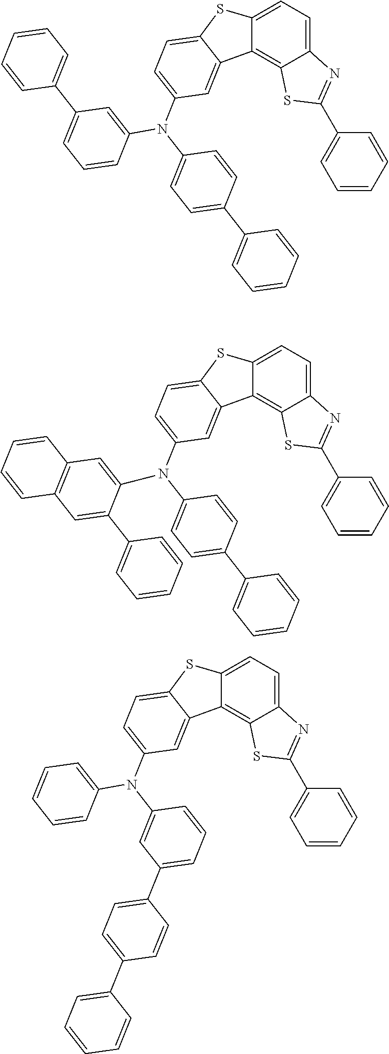

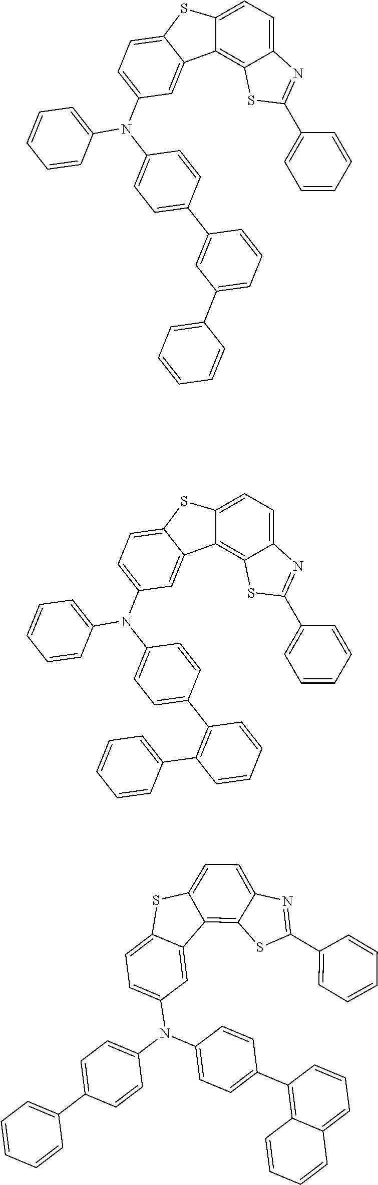 [SMILES: c1ccc(-c2cccc(-c3ccc(N(c4ccccc4)c4ccc5sc6ccc7nc(-c8ccccc8)sc7c6c5c4)cc3)c2)cc1 ; c1ccc(-c2nc3ccc4sc5ccc(N(c6ccccc6)c6ccc(-c7ccccc7-c7ccccc7)cc6)cc5c4c3s2)cc1 ; c1ccc(-c2ccc(N(c3ccc(-c4cccc5ccccc45)cc3)c3ccc4sc5ccc6nc(-c7ccccc7)sc6c5c4c3)cc2)cc1]

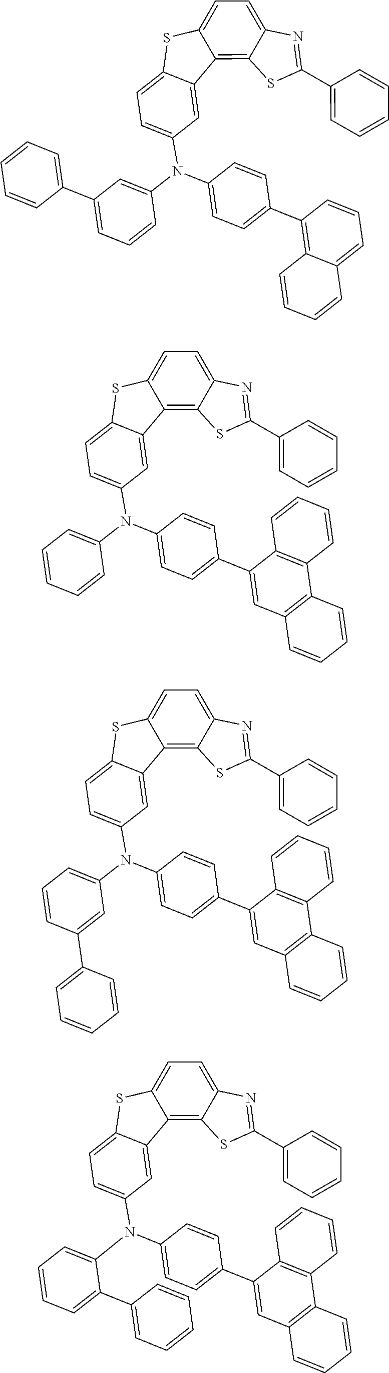 [SMILES: c1ccc(-c2cccc(N(c3ccc(-c4cccc5ccccc45)cc3)c3ccc4sc5ccc6nc(-c7ccccc7)sc6c5c4c3)c2)cc1 ; c1ccc(-c2nc3ccc4sc5ccc(N(c6ccccc6)c6ccc(-c7cc8ccccc8c8ccccc78)cc6)cc5c4c3s2)cc1 ; c1ccc(-c2cccc(N(c3ccc(-c4cc5ccccc5c5ccccc45)cc3)c3ccc4sc5ccc6nc(-c7ccccc7)sc6c5c4c3)c2)cc1 ; c1ccc(-c2nc3ccc4sc5ccc(N(c6ccc(-c7cc8ccccc8c8ccccc78)cc6)c6ccccc6-c6ccccc6)cc5c4c3s2)cc1]

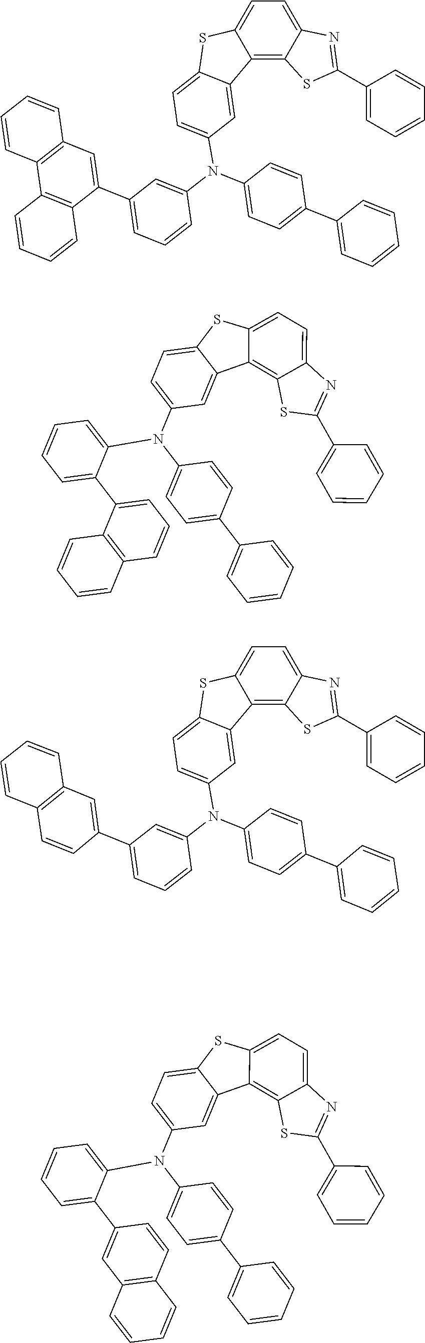

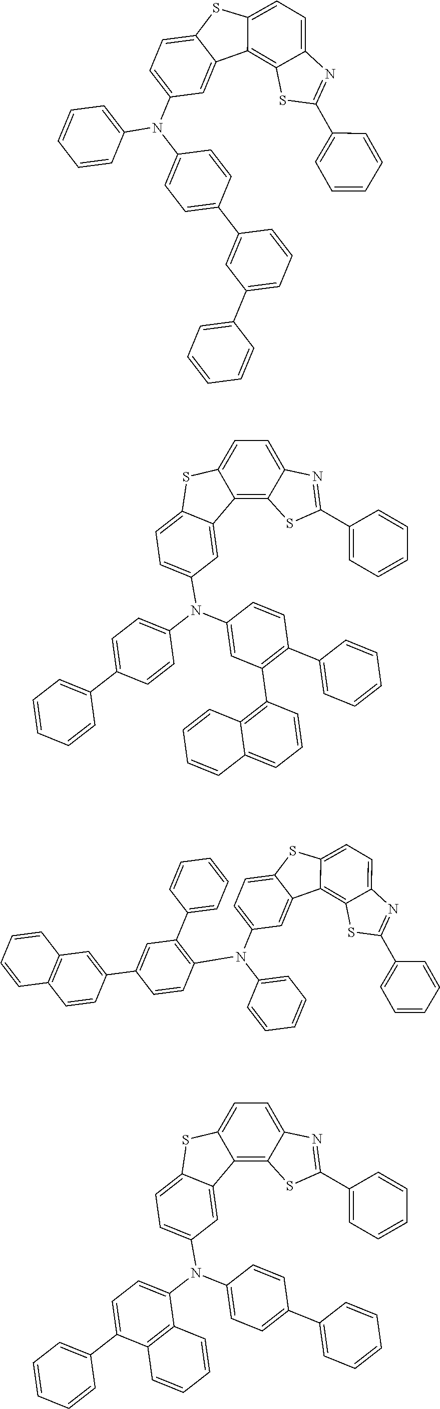 [SMILES: c1ccc(-c2cccc(-c3ccc(N(c4ccccc4)c4ccc5sc6ccc7nc(-c8ccccc8)sc7c6c5c4)cc3)c2)cc1 ; c1ccc(-c2ccc(N(c3ccc(-c4ccccc4)c(-c4cccc5ccccc45)c3)c3ccc4sc5ccc6nc(-c7ccccc7)sc6c5c4c3)cc2)cc1 ; c1ccc(-c2nc3ccc4sc5ccc(N(c6ccccc6)c6ccc(-c7ccc8ccccc8c7)cc6-c6ccccc6)cc5c4c3s2)cc1 ; c1ccc(-c2ccc(N(c3ccc4sc5ccc6nc(-c7ccccc7)sc6c5c4c3)c3ccc(-c4ccccc4)c4ccccc34)cc2)cc1]

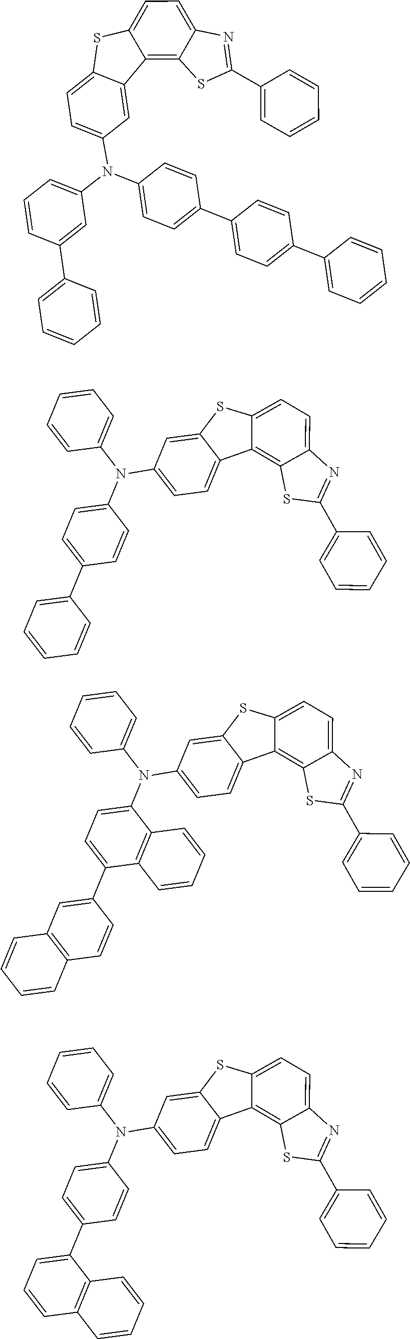

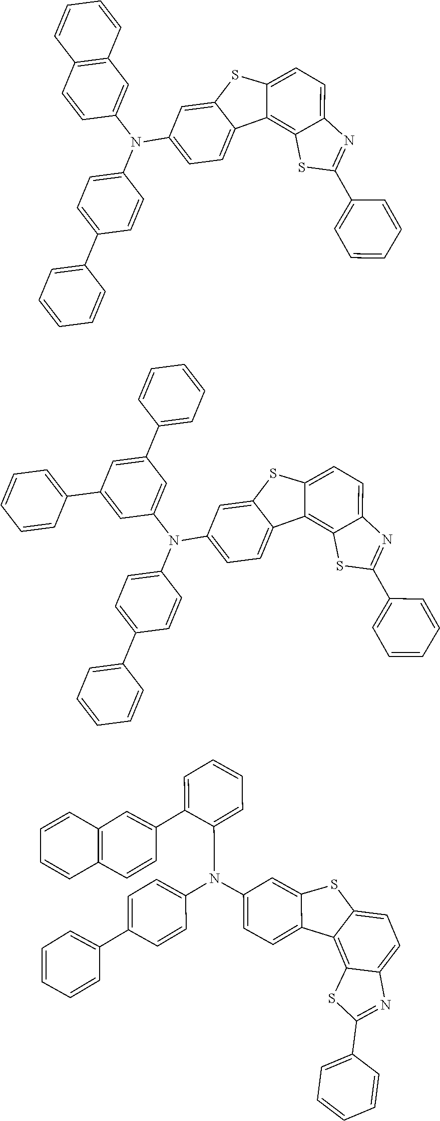 [SMILES: c1ccc(-c2ccc(N(c3ccc4ccccc4c3)c3ccc4c(c3)sc3ccc5nc(-c6ccccc6)sc5c34)cc2)cc1 ; c1ccc(-c2ccc(N(c3cc(-c4ccccc4)cc(-c4ccccc4)c3)c3ccc4c(c3)sc3ccc5nc(-c6ccccc6)sc5c34)cc2)cc1 ; c1ccc(-c2ccc(N(c3ccc4c(c3)sc3ccc5nc(-c6ccccc6)sc5c34)c3ccccc3-c3ccc4ccccc4c3)cc2)cc1]

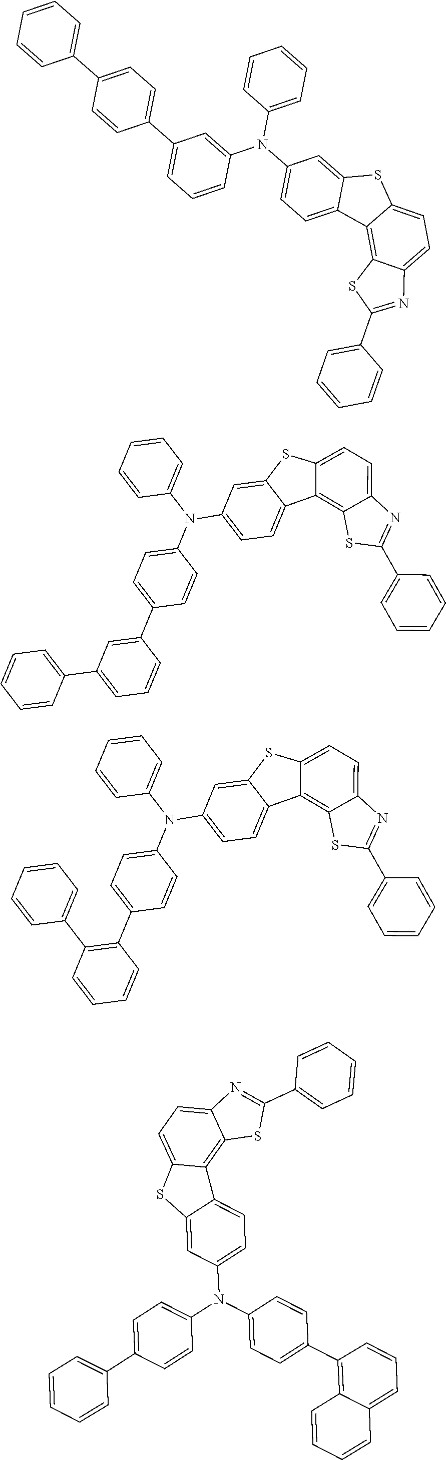

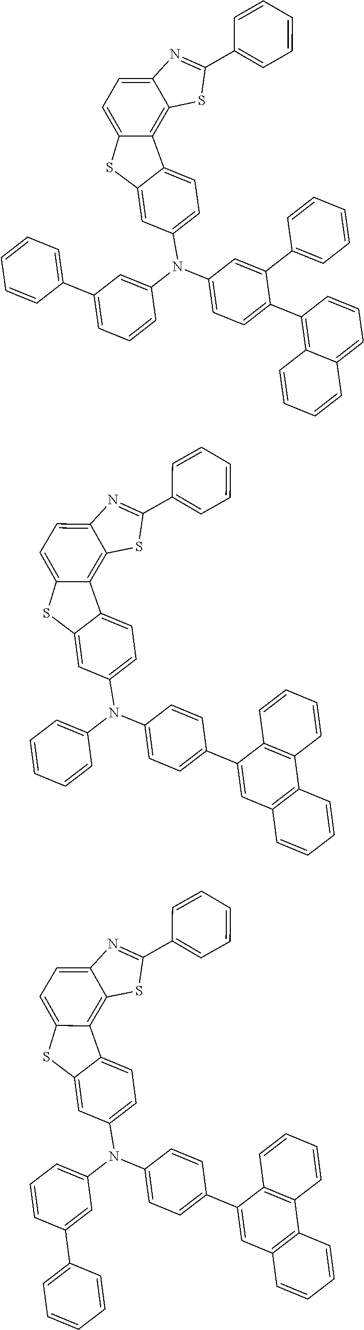 [SMILES: c1ccc(-c2cccc(N(c3ccc(-c4cccc5ccccc45)c(-c4ccccc4)c3)c3ccc4c(c3)sc3ccc5nc(-c6ccccc6)sc5c34)c2)cc1 ; c1ccc(-c2nc3ccc4sc5cc(N(c6ccccc6)c6ccc(-c7cc8ccccc8c8ccccc78)cc6)ccc5c4c3s2)cc1 ; c1ccc(-c2cccc(N(c3ccc(-c4cc5ccccc5c5ccccc45)cc3)c3ccc4c(c3)sc3ccc5nc(-c6ccccc6)sc5c34)c2)cc1]

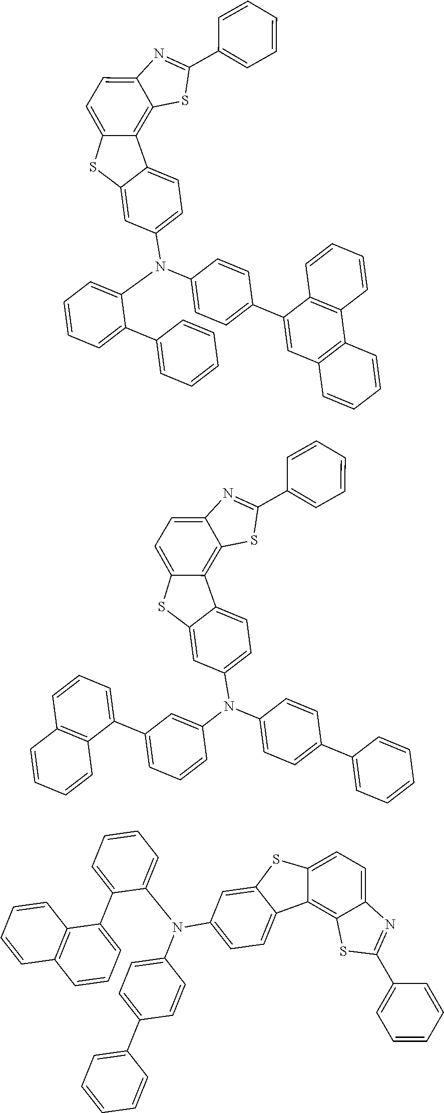

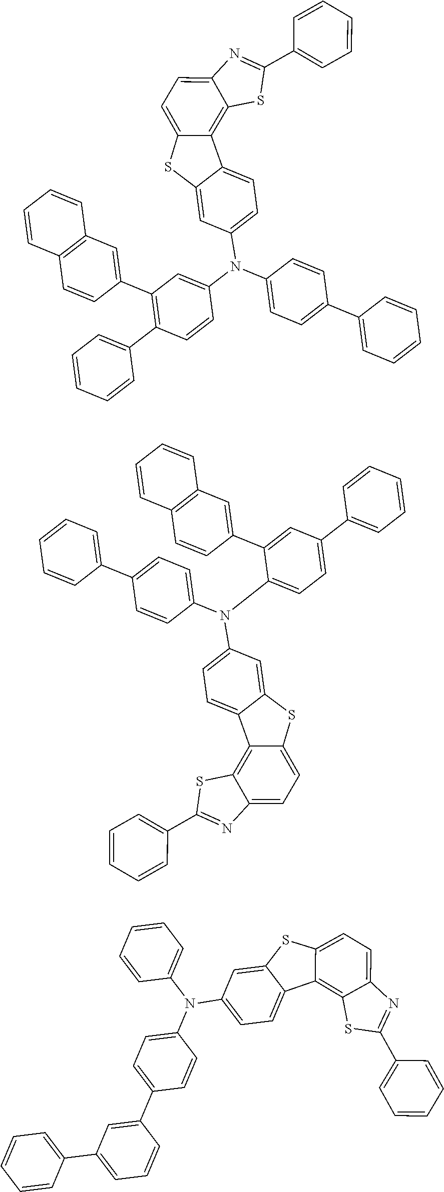

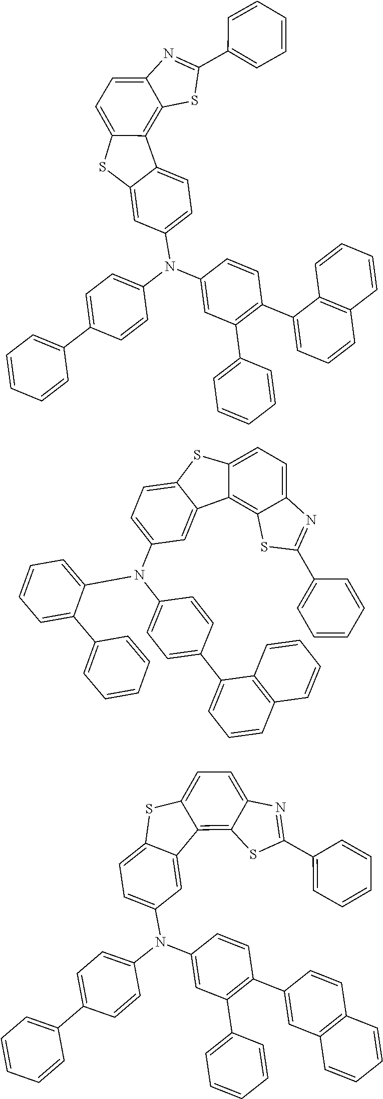

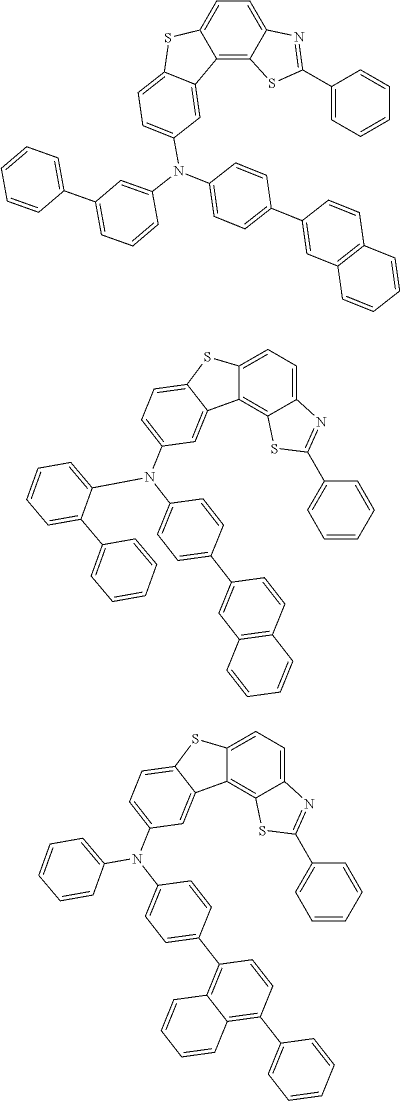

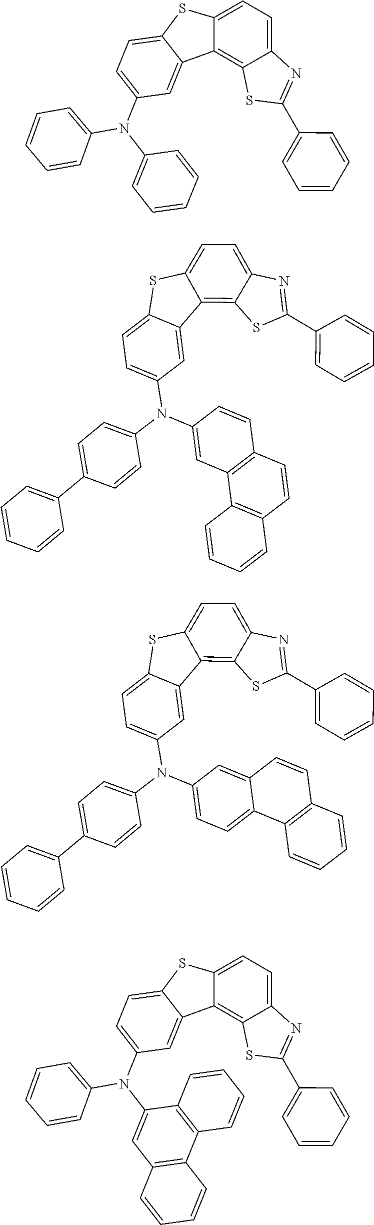

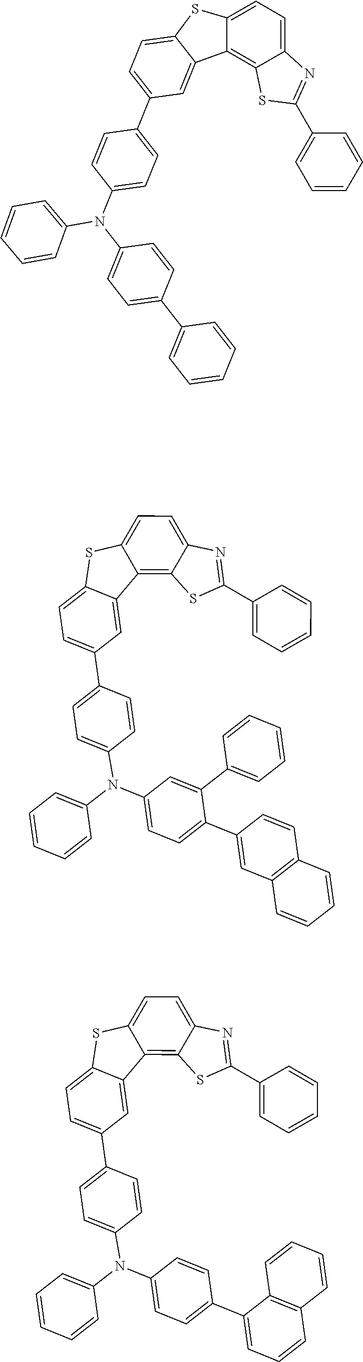 [SMILES: c1ccc(-c2ccc(N(c3ccccc3)c3ccc(-c4ccc5sc6ccc7nc(-c8ccccc8)sc7c6c5c4)cc3)cc2)cc1 ; c1ccc(-c2nc3ccc4sc5ccc(-c6ccc(N(c7ccccc7)c7ccc(-c8ccc9ccccc9c8)c(-c8ccccc8)c7)cc6)cc5c4c3s2)cc1 ; c1ccc(-c2nc3ccc4sc5ccc(-c6ccc(N(c7ccccc7)c7ccc(-c8cccc9ccccc89)cc7)cc6)cc5c4c3s2)cc1]

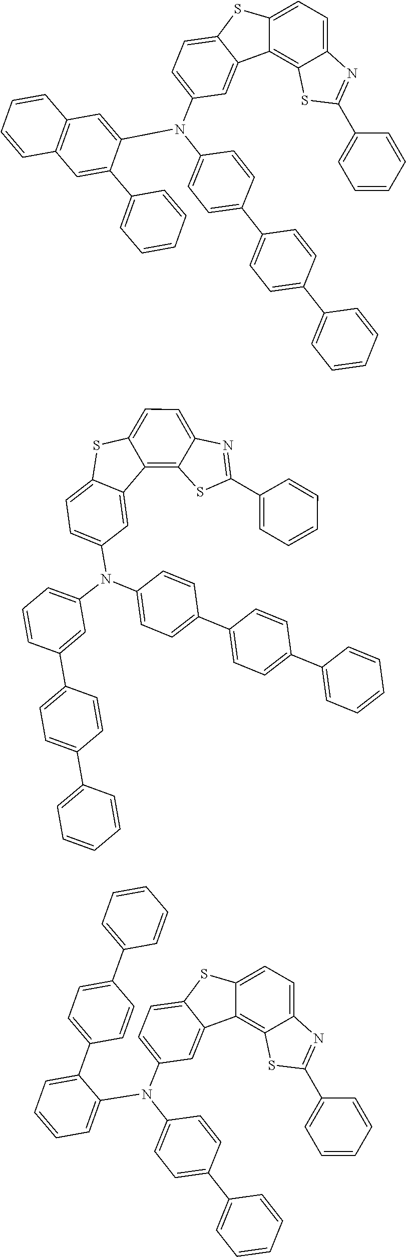

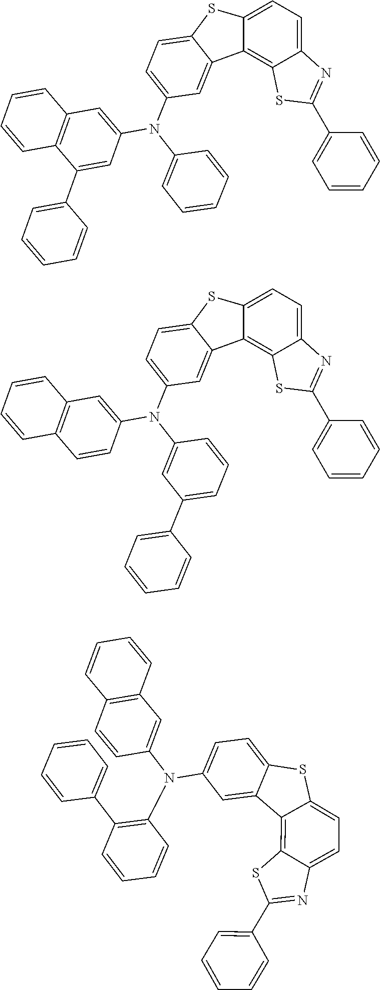 [SMILES: c1ccc(-c2nc3ccc4sc5ccc(N(c6ccccc6)c6cc(-c7ccccc7)c7ccccc7c6)cc5c4c3s2)cc1 ; c1ccc(-c2cccc(N(c3ccc4ccccc4c3)c3ccc4sc5ccc6nc(-c7ccccc7)sc6c5c4c3)c2)cc1 ; c1ccc(-c2nc3ccc4sc5ccc(N(c6ccc7ccccc7c6)c6ccccc6-c6ccccc6)cc5c4c3s2)cc1]

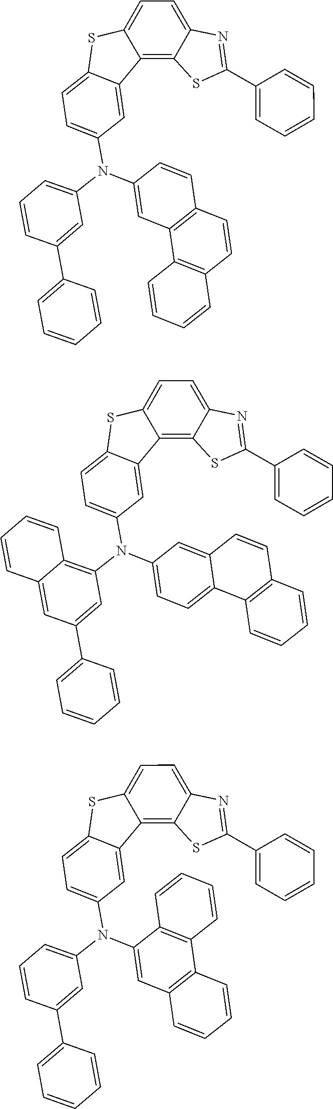 [SMILES: c1ccc(-c2cccc(N(c3ccc4ccc5ccccc5c4c3)c3ccc4sc5ccc6nc(-c7ccccc7)sc6c5c4c3)c2)cc1 ; c1ccc(-c2cc(N(c3ccc4c(ccc5ccccc54)c3)c3ccc4sc5ccc6nc(-c7ccccc7)sc6c5c4c3)c3ccccc3c2)cc1 ; c1ccc(-c2cccc(N(c3ccc4sc5ccc6nc(-c7ccccc7)sc6c5c4c3)c3cc4ccccc4c4ccccc34)c2)cc1]

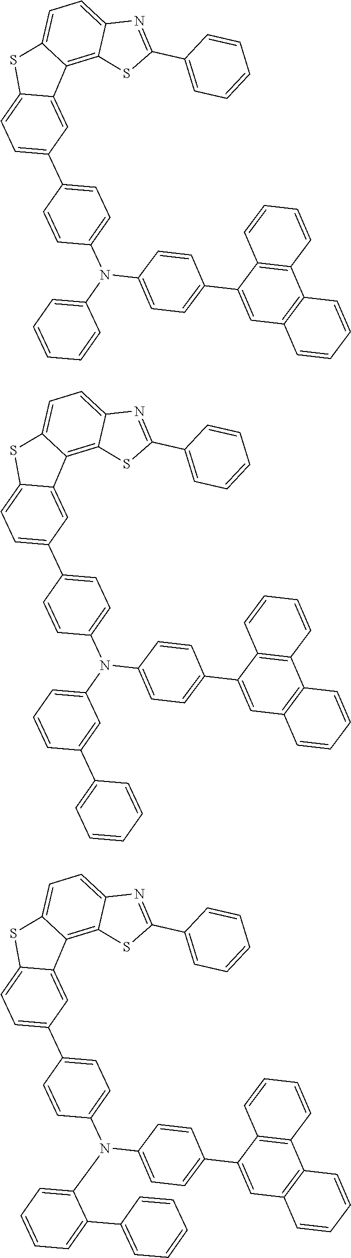

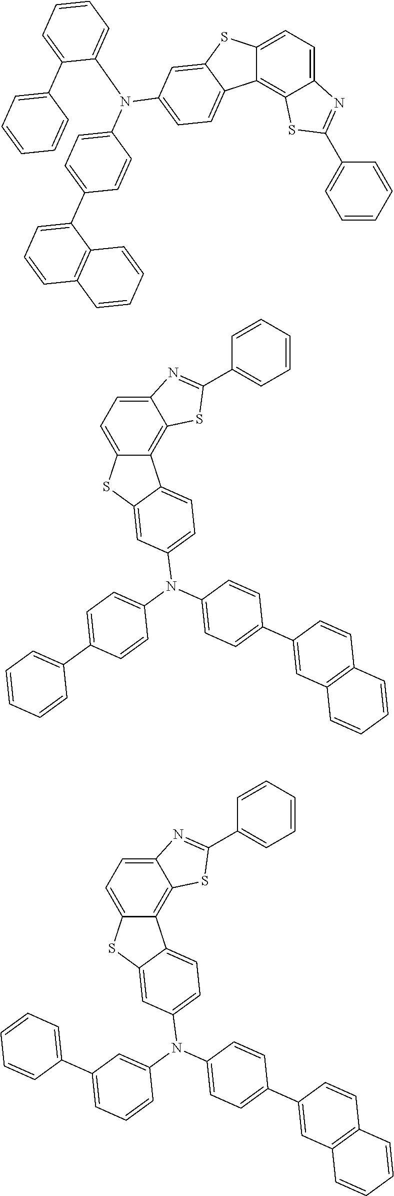 [SMILES: c1ccc(-c2nc3ccc4sc5cc(N(c6ccc(-c7cccc8ccccc78)cc6)c6ccccc6-c6ccccc6)ccc5c4c3s2)cc1 ; c1ccc(-c2ccc(N(c3ccc(-c4ccc5ccccc5c4)cc3)c3ccc4c(c3)sc3ccc5nc(-c6ccccc6)sc5c34)cc2)cc1 ; c1ccc(-c2cccc(N(c3ccc(-c4ccc5ccccc5c4)cc3)c3ccc4c(c3)sc3ccc5nc(-c6ccccc6)sc5c34)c2)cc1]

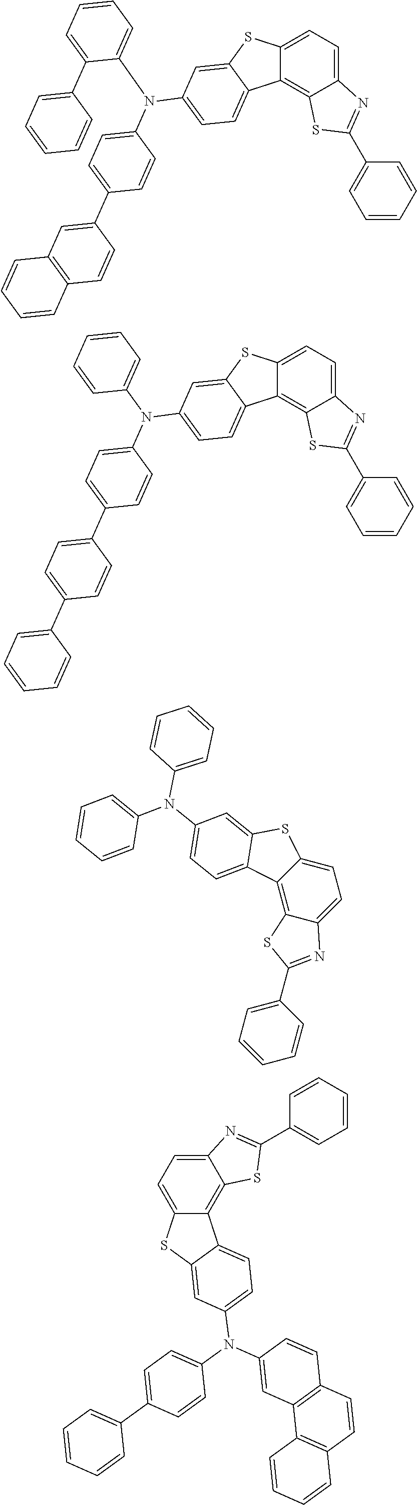

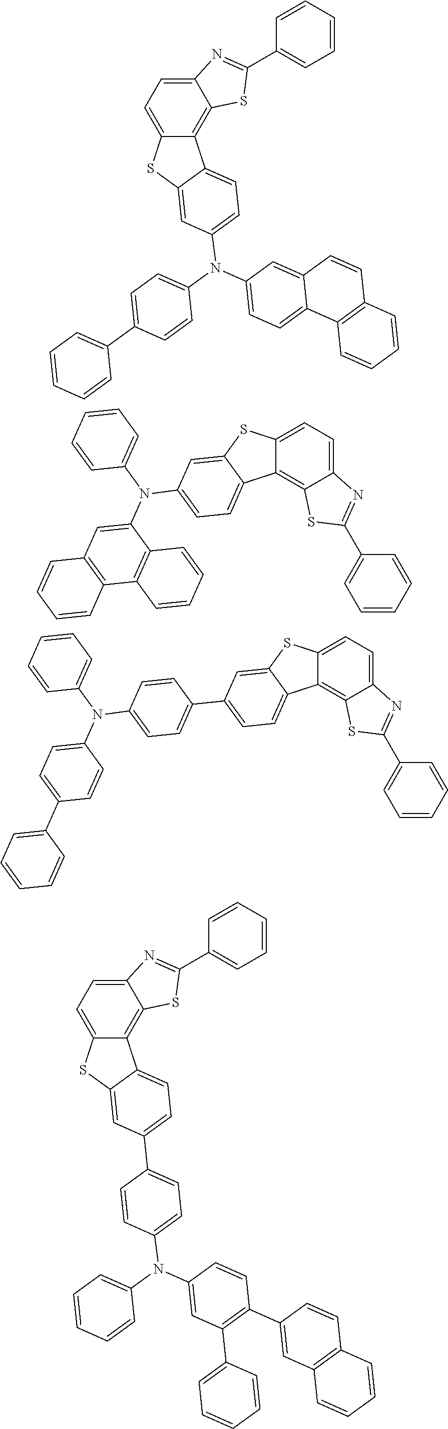

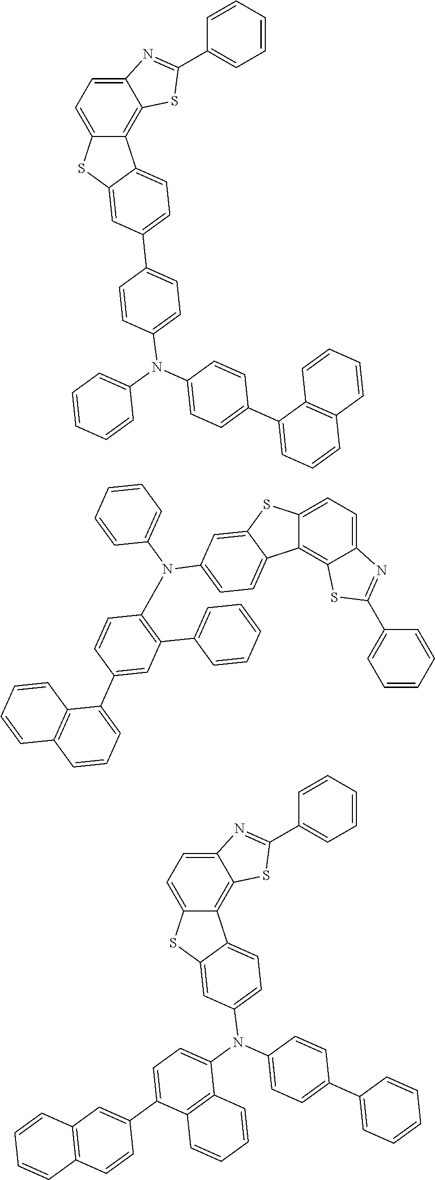

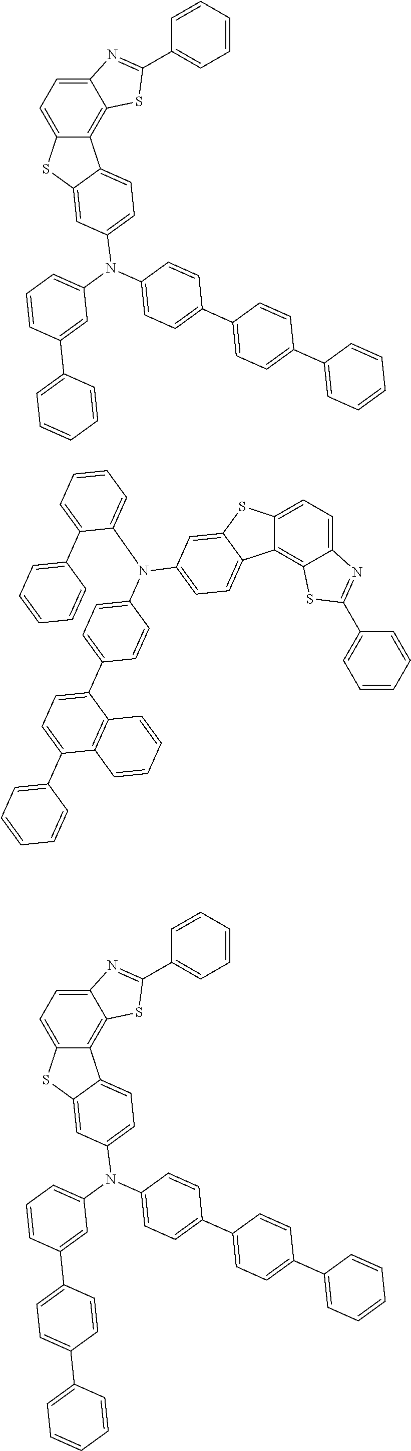

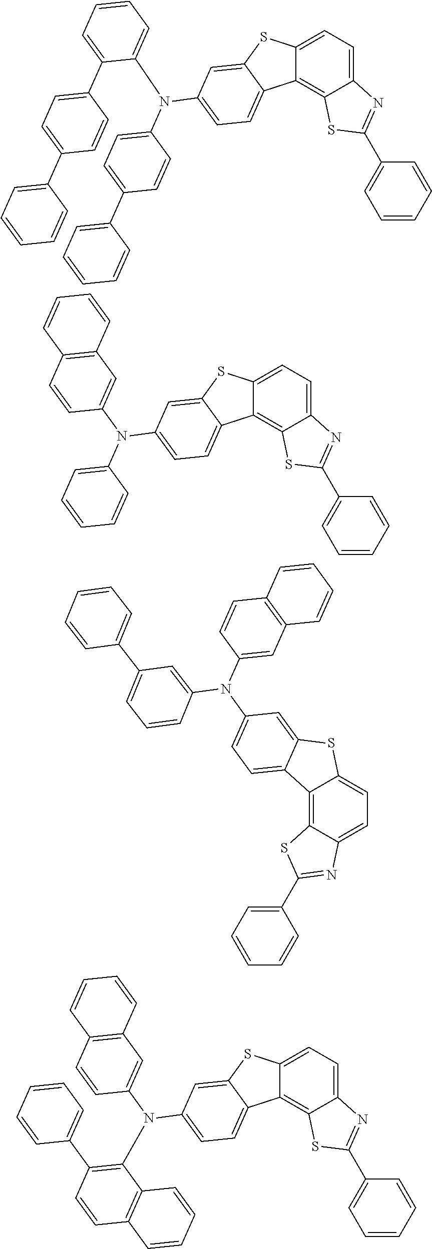

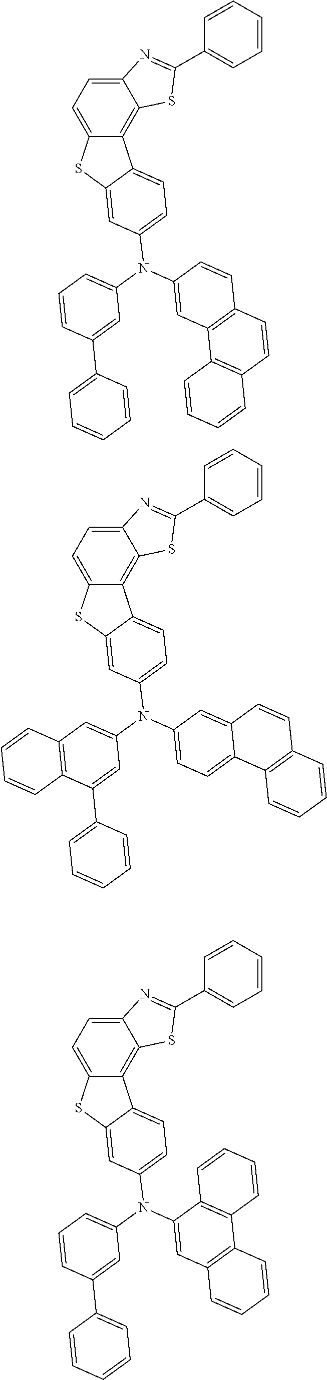

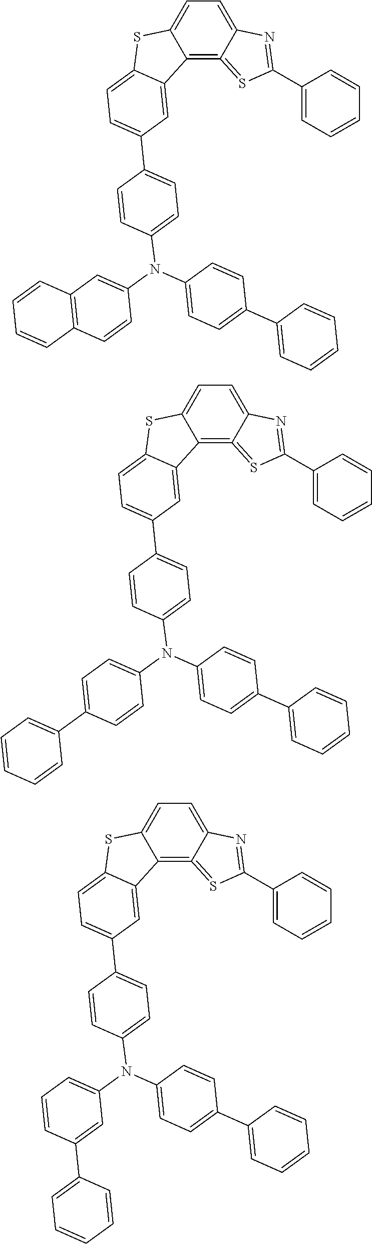

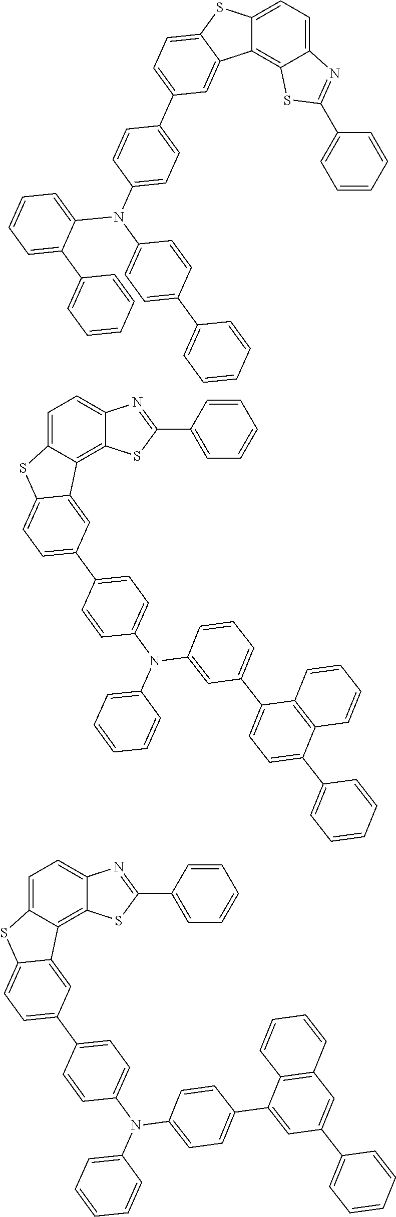

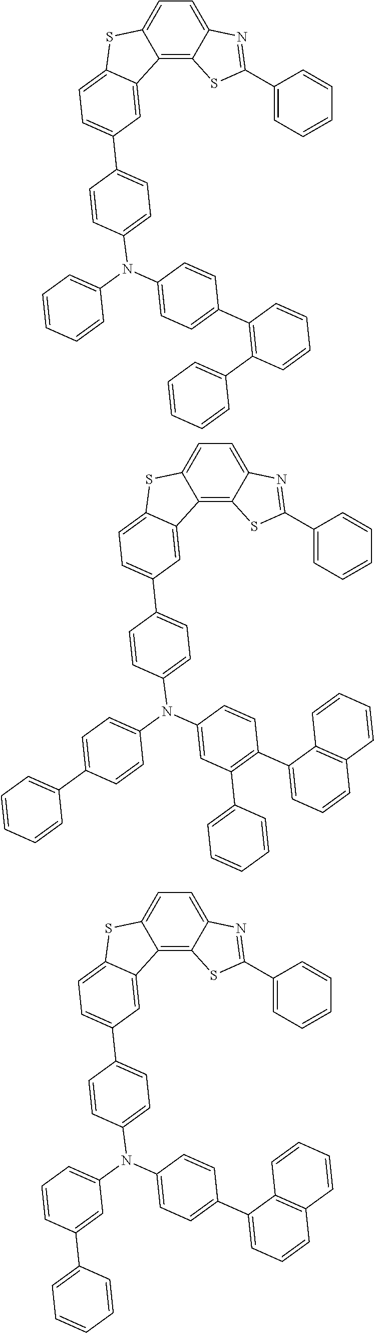

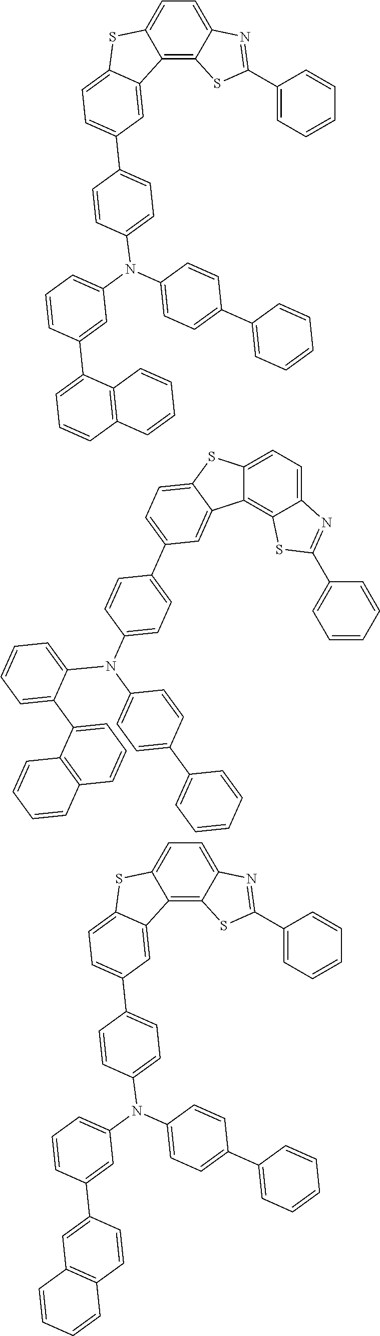

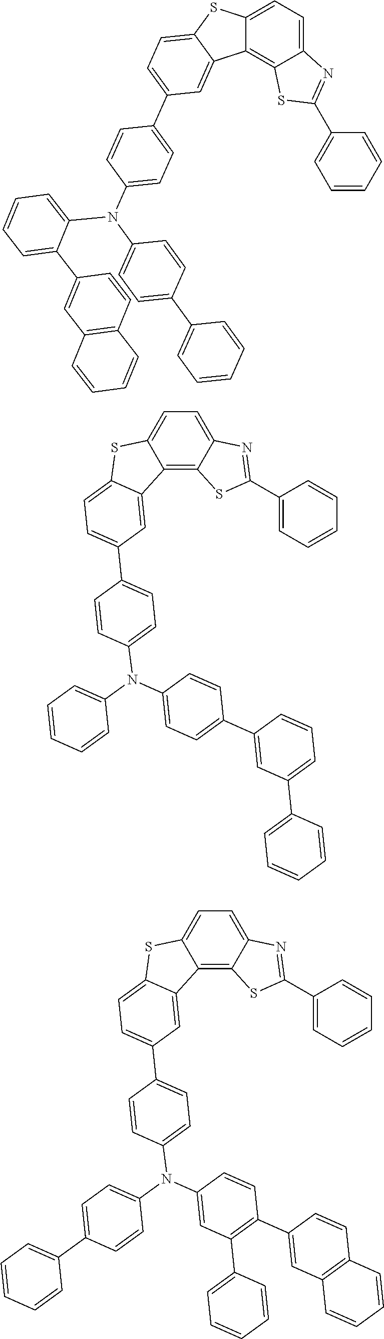

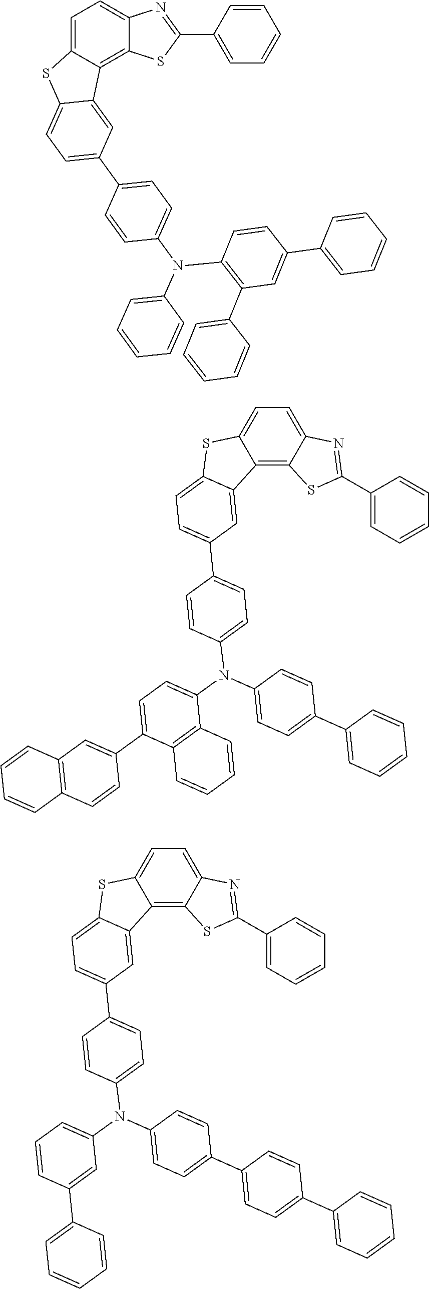

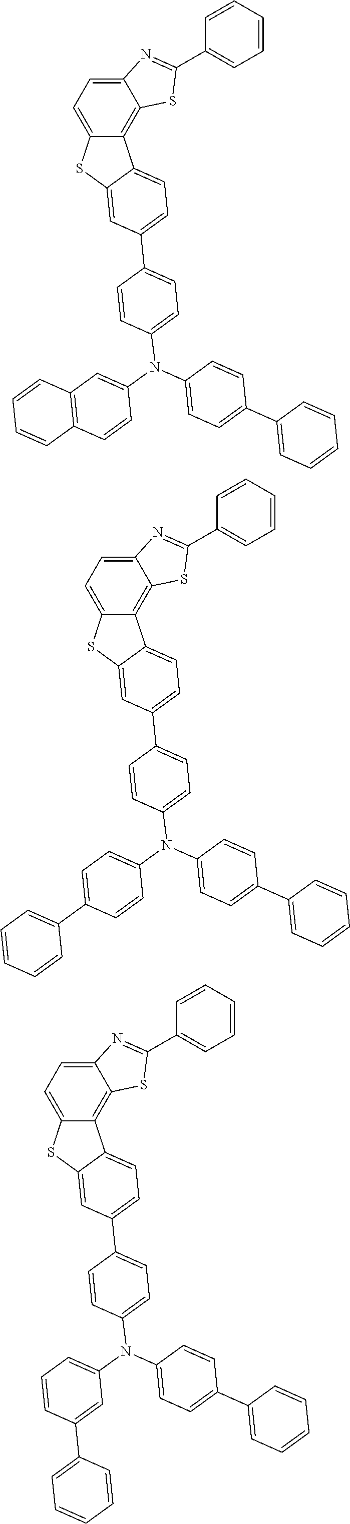

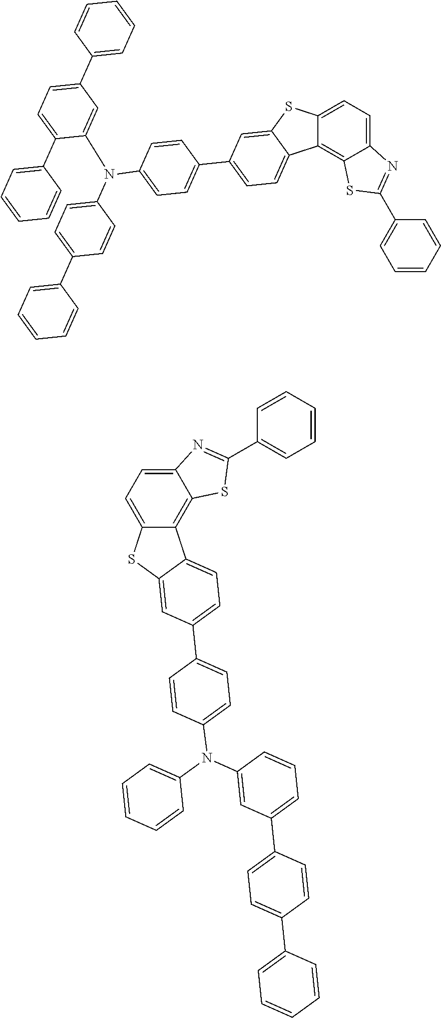 [SMILES: c1ccc(-c2ccc(N(c3ccc(-c4ccc5c(c4)sc4ccc6nc(-c7ccccc7)sc6c45)cc3)c3cc(-c4ccccc4)ccc3-c3ccccc3)cc2)cc1 ; c1ccc(-c2ccc(-c3cccc(N(c4ccccc4)c4ccc(-c5ccc6c(c5)sc5ccc7nc(-c8ccccc8)sc7c56)cc4)c3)cc2)cc1]

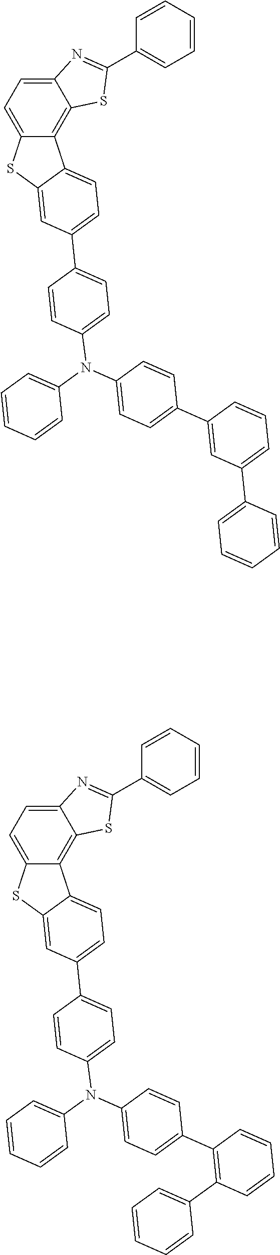 [SMILES: c1ccc(-c2cccc(-c3ccc(N(c4ccccc4)c4ccc(-c5ccc6c(c5)sc5ccc7nc(-c8ccccc8)sc7c56)cc4)cc3)c2)cc1 ; c1ccc(-c2nc3ccc4sc5cc(-c6ccc(N(c7ccccc7)c7ccc(-c8ccccc8-c8ccccc8)cc7)cc6)ccc5c4c3s2)cc1]

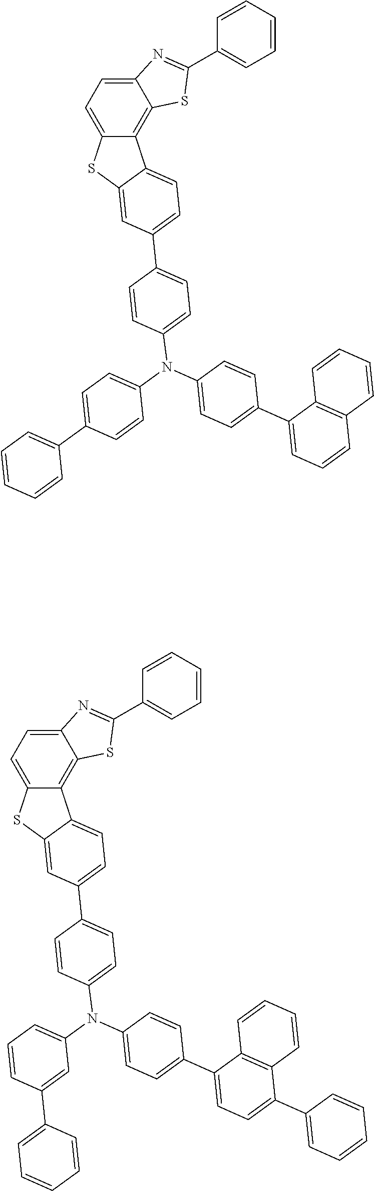 [SMILES: c1ccc(-c2ccc(N(c3ccc(-c4ccc5c(c4)sc4ccc6nc(-c7ccccc7)sc6c45)cc3)c3ccc(-c4cccc5ccccc45)cc3)cc2)cc1 ; c1ccc(-c2cccc(N(c3ccc(-c4ccc5c(c4)sc4ccc6nc(-c7ccccc7)sc6c45)cc3)c3ccc(-c4ccc(-c5ccccc5)c5ccccc45)cc3)c2)cc1]

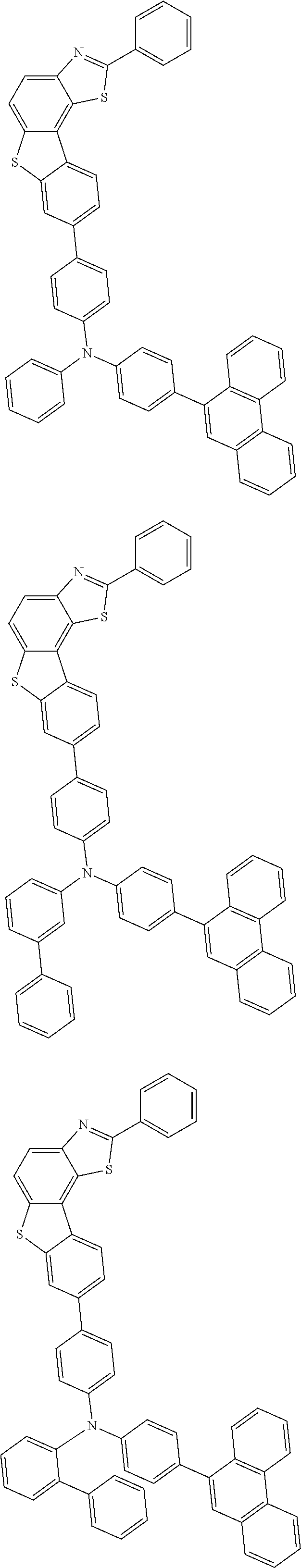 [SMILES: c1ccc(-c2nc3ccc4sc5cc(-c6ccc(N(c7ccccc7)c7ccc(-c8cc9ccccc9c9ccccc89)cc7)cc6)ccc5c4c3s2)cc1 ; c1ccc(-c2cccc(N(c3ccc(-c4ccc5c(c4)sc4ccc6nc(-c7ccccc7)sc6c45)cc3)c3ccc(-c4cc5ccccc5c5ccccc45)cc3)c2)cc1 ; c1ccc(-c2nc3ccc4sc5cc(-c6ccc(N(c7ccc(-c8cc9ccccc9c9ccccc89)cc7)c7ccccc7-c7ccccc7)cc6)ccc5c4c3s2)cc1]

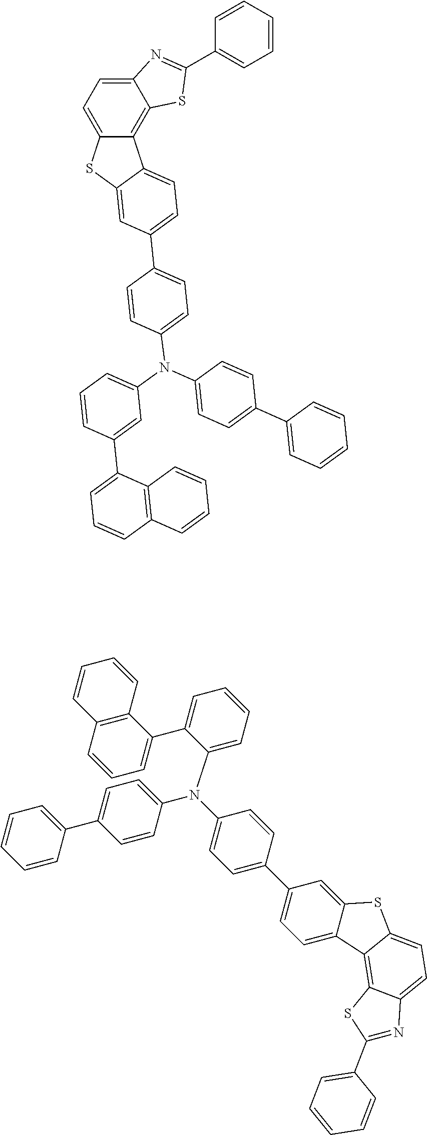 [SMILES: c1ccc(-c2ccc(N(c3ccc(-c4ccc5c(c4)sc4ccc6nc(-c7ccccc7)sc6c45)cc3)c3cccc(-c4cccc5ccccc45)c3)cc2)cc1 ; c1ccc(-c2ccc(N(c3ccc(-c4ccc5c(c4)sc4ccc6nc(-c7ccccc7)sc6c45)cc3)c3ccccc3-c3cccc4ccccc34)cc2)cc1]

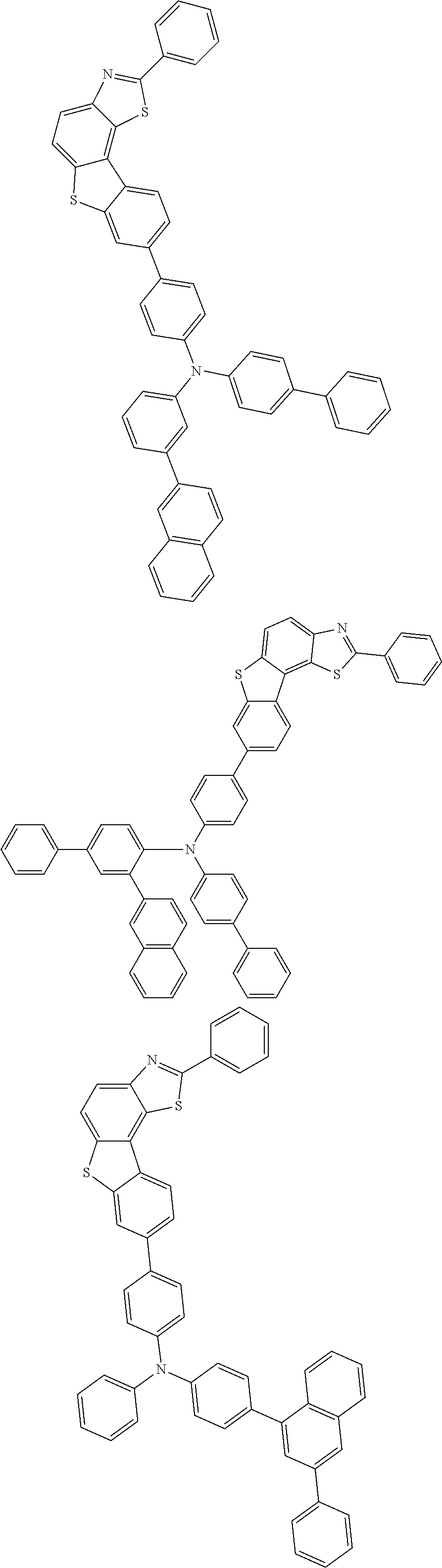

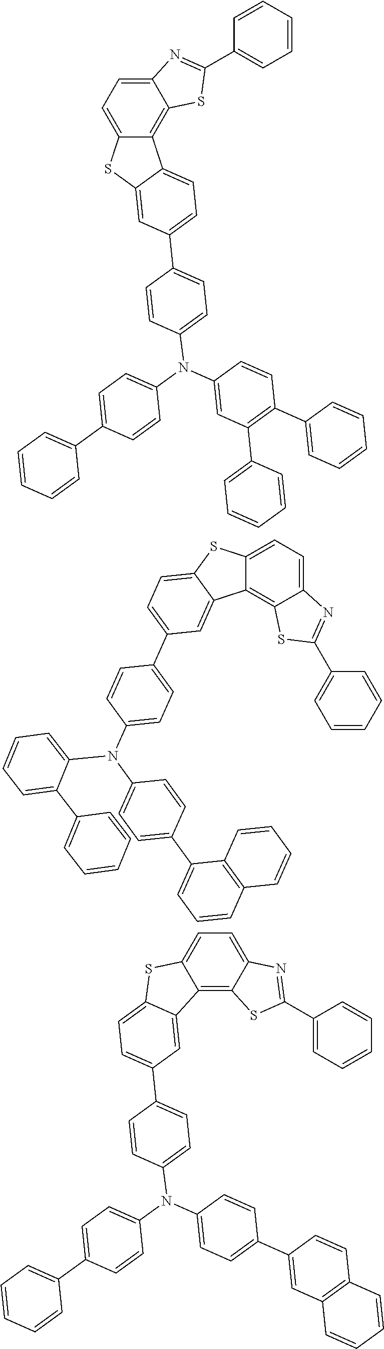

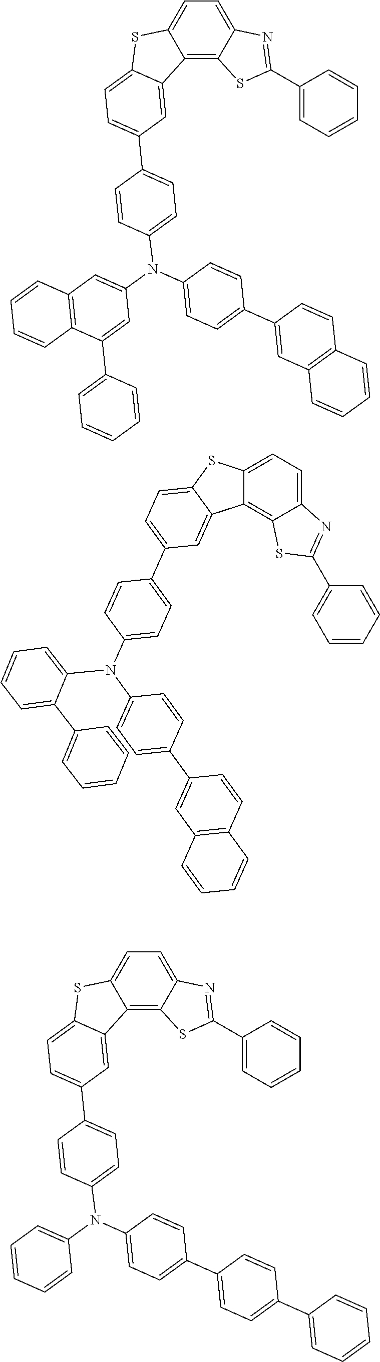 [SMILES: c1ccc(-c2nc3ccc4sc5ccc(-c6ccc(N(c7ccc(-c8ccc9ccccc9c8)cc7)c7cc(-c8ccccc8)c8ccccc8c7)cc6)cc5c4c3s2)cc1 ; c1ccc(-c2nc3ccc4sc5ccc(-c6ccc(N(c7ccc(-c8ccc9ccccc9c8)cc7)c7ccccc7-c7ccccc7)cc6)cc5c4c3s2)cc1 ; c1ccc(-c2ccc(-c3ccc(N(c4ccccc4)c4ccc(-c5ccc6sc7ccc8nc(-c9ccccc9)sc8c7c6c5)cc4)cc3)cc2)cc1]

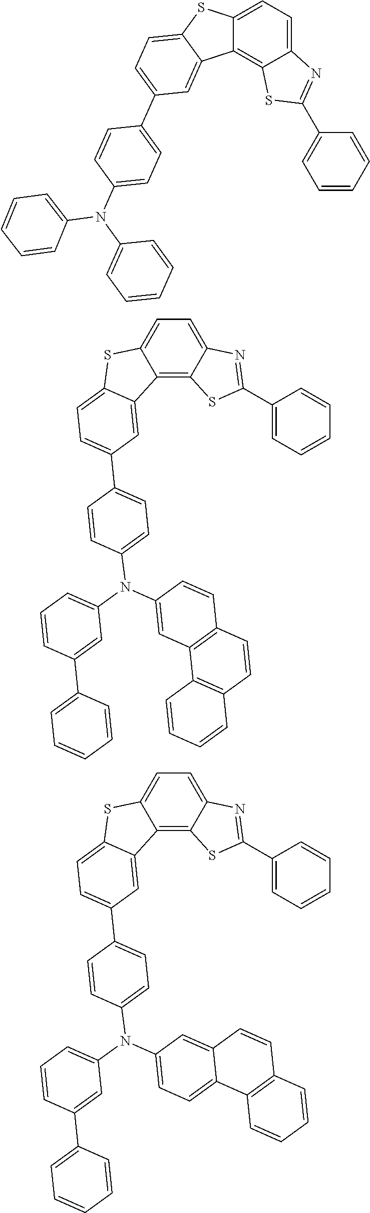

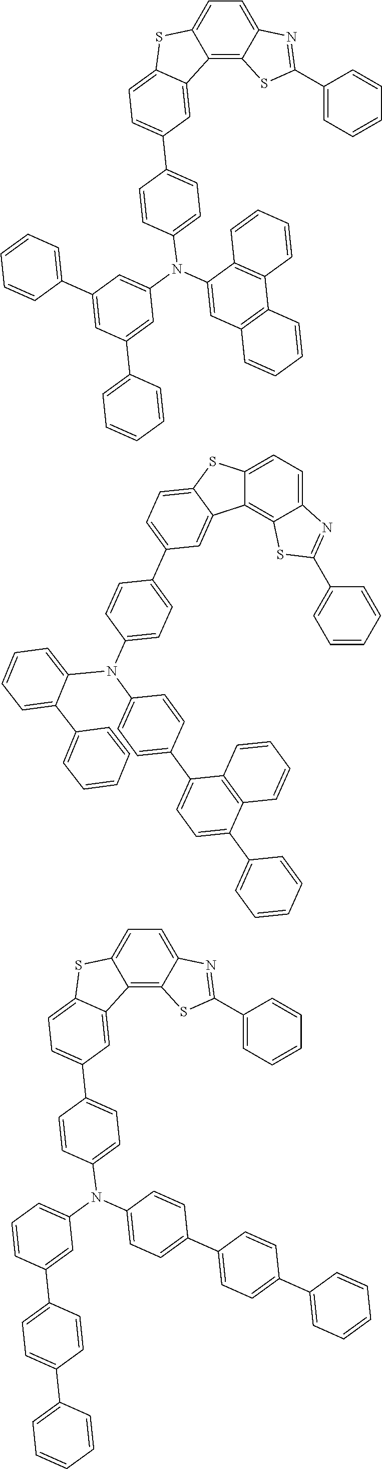 [SMILES: c1ccc(-c2cc(-c3ccccc3)cc(N(c3ccc(-c4ccc5sc6ccc7nc(-c8ccccc8)sc7c6c5c4)cc3)c3cc4ccccc4c4ccccc34)c2)cc1 ; c1ccc(-c2nc3ccc4sc5ccc(-c6ccc(N(c7ccc(-c8ccc(-c9ccccc9)c9ccccc89)cc7)c7ccccc7-c7ccccc7)cc6)cc5c4c3s2)cc1 ; c1ccc(-c2ccc(-c3ccc(N(c4ccc(-c5ccc6sc7ccc8nc(-c9ccccc9)sc8c7c6c5)cc4)c4cccc(-c5ccc(-c6ccccc6)cc5)c4)cc3)cc2)cc1]

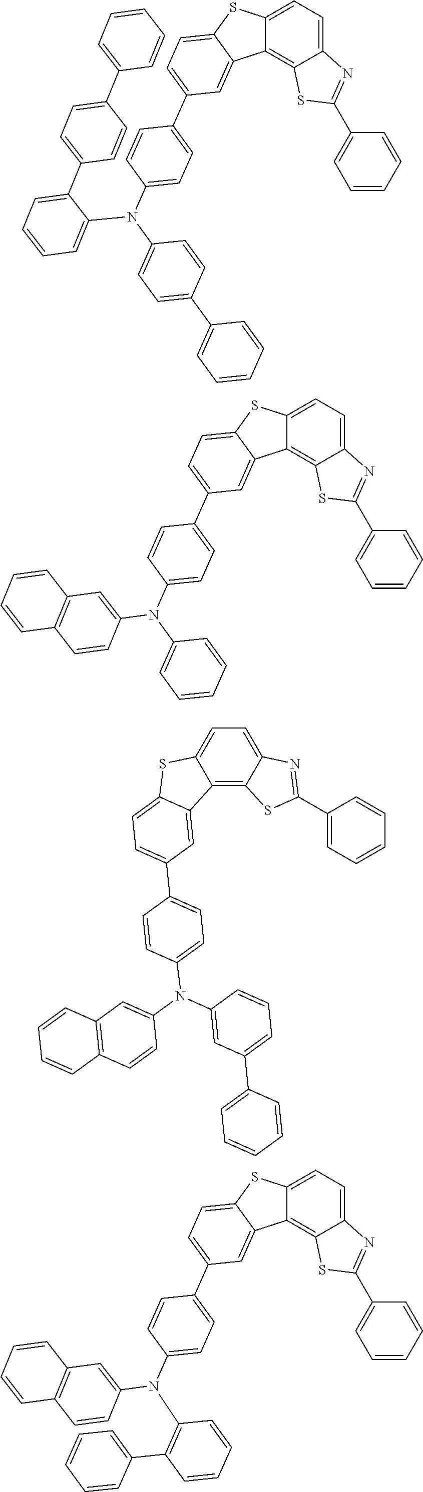

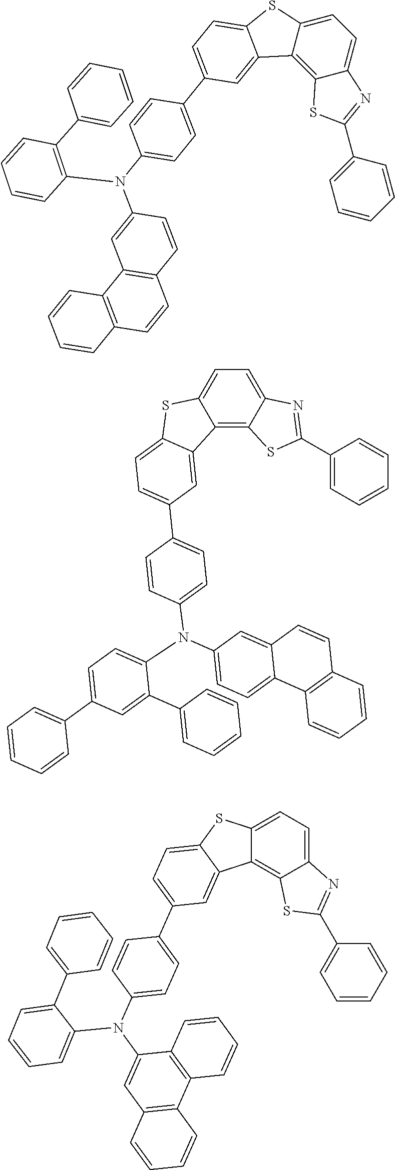 [SMILES: c1ccc(-c2nc3ccc4sc5ccc(-c6ccc(N(c7ccc8ccc9ccccc9c8c7)c7ccccc7-c7ccccc7)cc6)cc5c4c3s2)cc1 ; c1ccc(-c2ccc(N(c3ccc(-c4ccc5sc6ccc7nc(-c8ccccc8)sc7c6c5c4)cc3)c3ccc4c(ccc5ccccc54)c3)c(-c3ccccc3)c2)cc1 ; c1ccc(-c2nc3ccc4sc5ccc(-c6ccc(N(c7ccccc7-c7ccccc7)c7cc8ccccc8c8ccccc78)cc6)cc5c4c3s2)cc1]

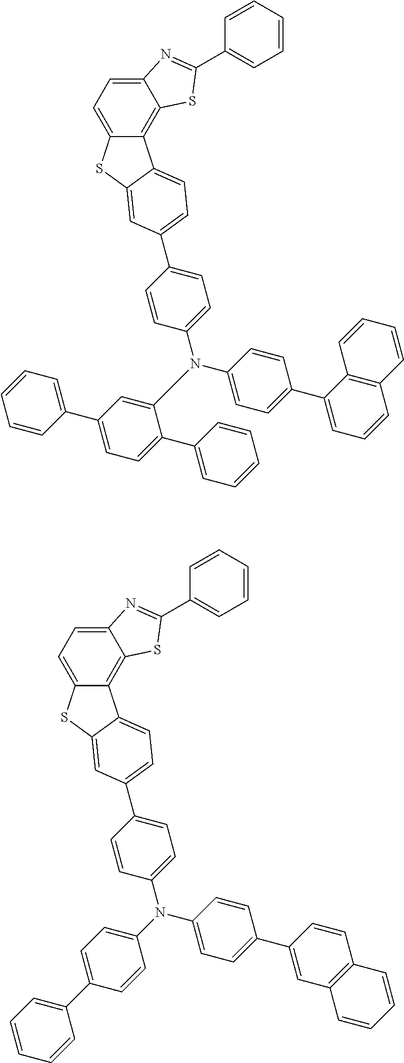 [SMILES: c1ccc(-c2ccc(-c3ccccc3)c(N(c3ccc(-c4ccc5c(c4)sc4ccc6nc(-c7ccccc7)sc6c45)cc3)c3ccc(-c4cccc5ccccc45)cc3)c2)cc1 ; c1ccc(-c2ccc(N(c3ccc(-c4ccc5ccccc5c4)cc3)c3ccc(-c4ccc5c(c4)sc4ccc6nc(-c7ccccc7)sc6c45)cc3)cc2)cc1]

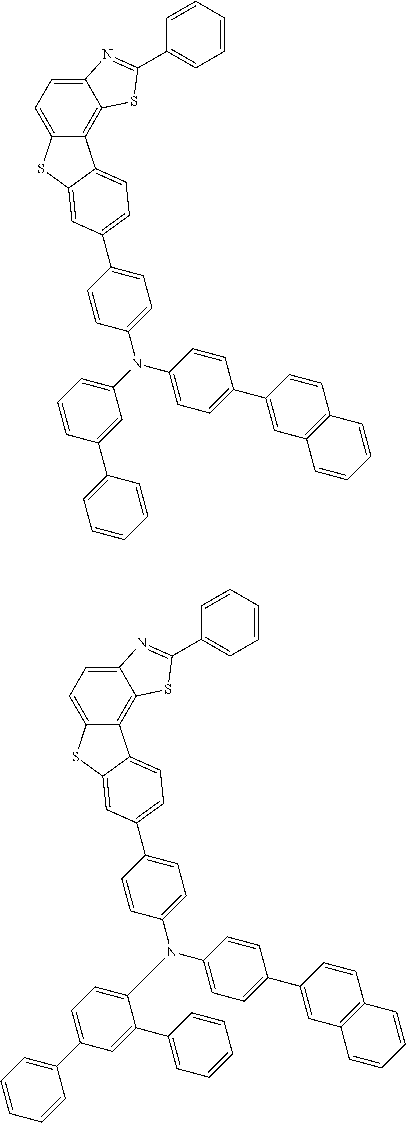 [SMILES: c1ccc(-c2cccc(N(c3ccc(-c4ccc5ccccc5c4)cc3)c3ccc(-c4ccc5c(c4)sc4ccc6nc(-c7ccccc7)sc6c45)cc3)c2)cc1 ; c1ccc(-c2ccc(N(c3ccc(-c4ccc5ccccc5c4)cc3)c3ccc(-c4ccc5c(c4)sc4ccc6nc(-c7ccccc7)sc6c45)cc3)c(-c3ccccc3)c2)cc1]

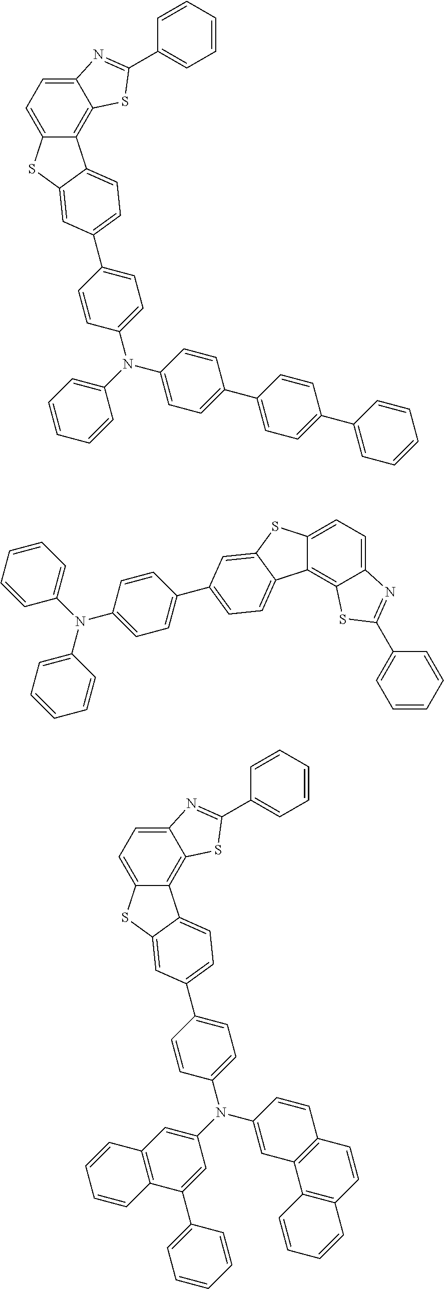 [SMILES: c1ccc(-c2ccc(-c3ccc(N(c4ccccc4)c4ccc(-c5ccc6c(c5)sc5ccc7nc(-c8ccccc8)sc7c56)cc4)cc3)cc2)cc1 ; c1ccc(-c2nc3ccc4sc5cc(-c6ccc(N(c7ccccc7)c7ccccc7)cc6)ccc5c4c3s2)cc1 ; c1ccc(-c2nc3ccc4sc5cc(-c6ccc(N(c7cc(-c8ccccc8)c8ccccc8c7)c7ccc8ccc9ccccc9c8c7)cc6)ccc5c4c3s2)cc1]

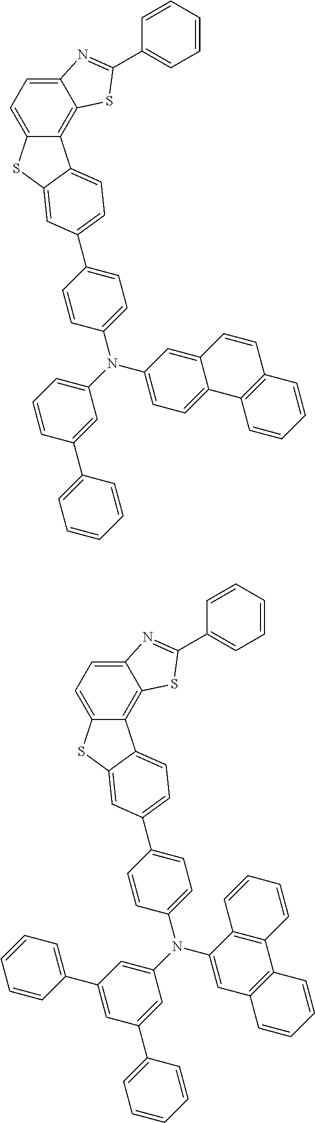 [SMILES: c1ccc(-c2cccc(N(c3ccc(-c4ccc5c(c4)sc4ccc6nc(-c7ccccc7)sc6c45)cc3)c3ccc4c(ccc5ccccc54)c3)c2)cc1 ; c1ccc(-c2cc(-c3ccccc3)cc(N(c3ccc(-c4ccc5c(c4)sc4ccc6nc(-c7ccccc7)sc6c45)cc3)c3cc4ccccc4c4ccccc34)c2)cc1]

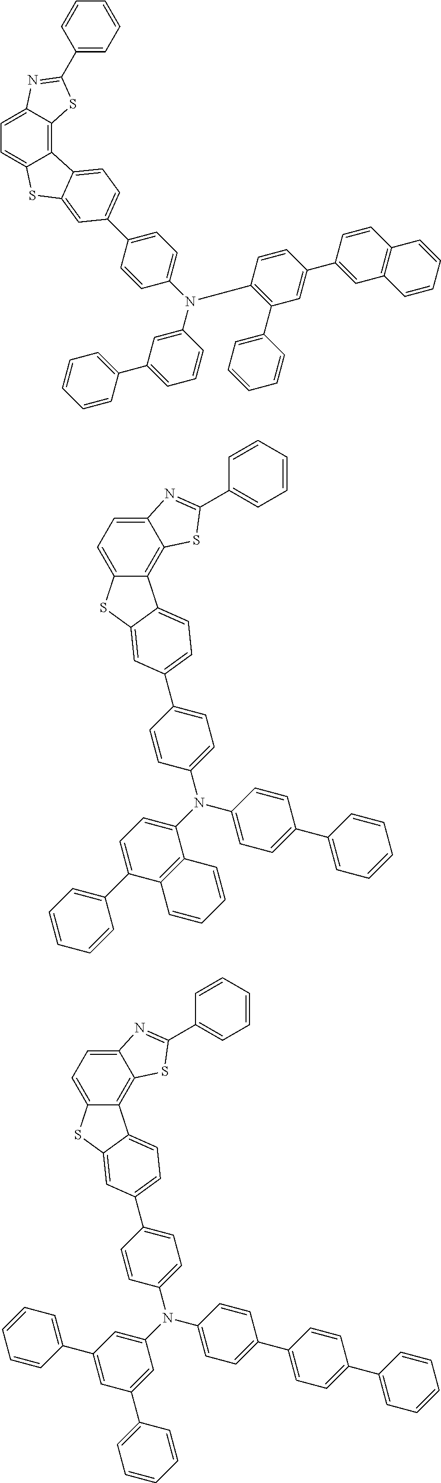 [SMILES: c1ccc(-c2cccc(N(c3ccc(-c4ccc5c(c4)sc4ccc6nc(-c7ccccc7)sc6c45)cc3)c3ccc(-c4ccc5ccccc5c4)cc3-c3ccccc3)c2)cc1 ; c1ccc(-c2ccc(N(c3ccc(-c4ccc5c(c4)sc4ccc6nc(-c7ccccc7)sc6c45)cc3)c3ccc(-c4ccccc4)c4ccccc34)cc2)cc1 ; c1ccc(-c2ccc(-c3ccc(N(c4ccc(-c5ccc6c(c5)sc5ccc7nc(-c8ccccc8)sc7c56)cc4)c4cc(-c5ccccc5)cc(-c5ccccc5)c4)cc3)cc2)cc1]

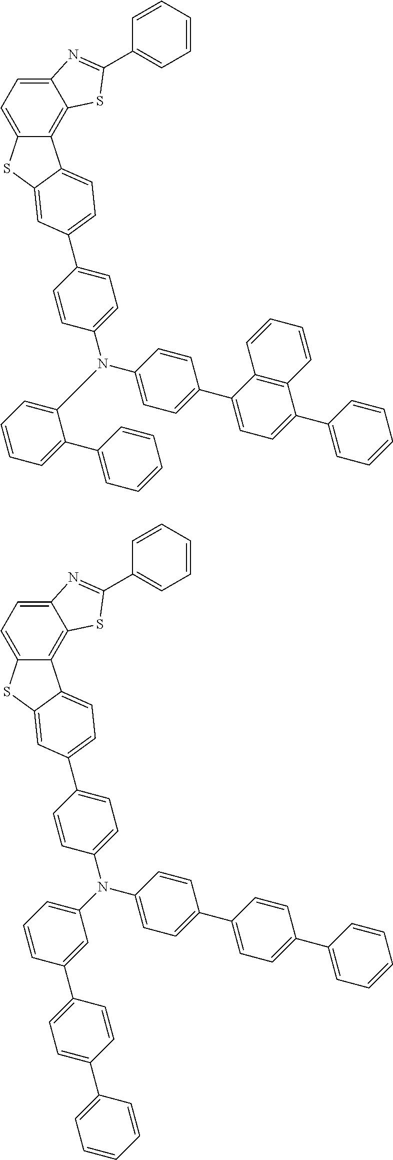

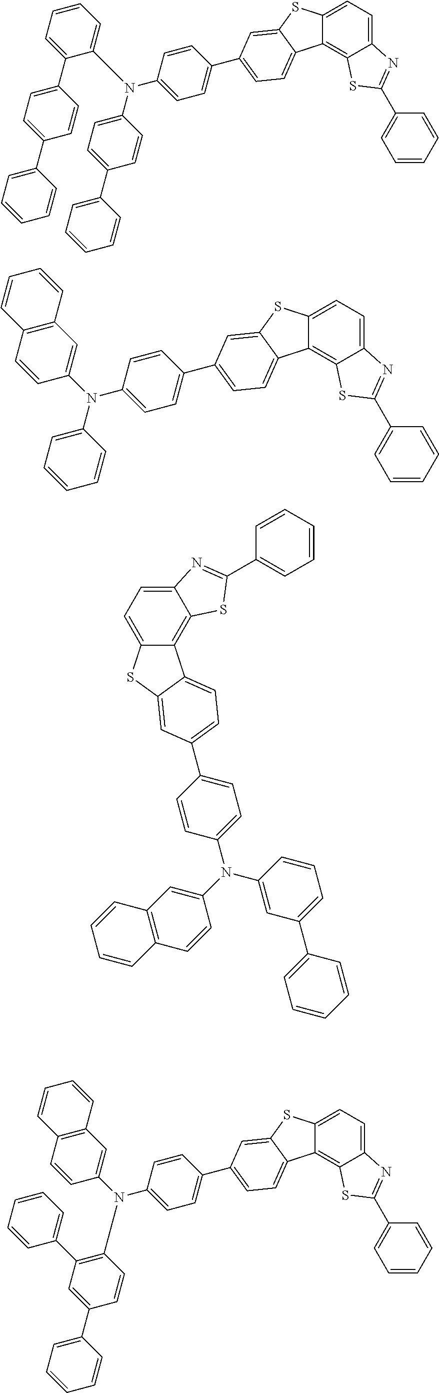 [SMILES: c1ccc(-c2ccc(-c3ccccc3N(c3ccc(-c4ccccc4)cc3)c3ccc(-c4ccc5c(c4)sc4ccc6nc(-c7ccccc7)sc6c45)cc3)cc2)cc1 ; c1ccc(-c2nc3ccc4sc5cc(-c6ccc(N(c7ccccc7)c7ccc8ccccc8c7)cc6)ccc5c4c3s2)cc1 ; c1ccc(-c2cccc(N(c3ccc(-c4ccc5c(c4)sc4ccc6nc(-c7ccccc7)sc6c45)cc3)c3ccc4ccccc4c3)c2)cc1 ; c1ccc(-c2ccc(N(c3ccc(-c4ccc5c(c4)sc4ccc6nc(-c7ccccc7)sc6c45)cc3)c3ccc4ccccc4c3)c(-c3ccccc3)c2)cc1]

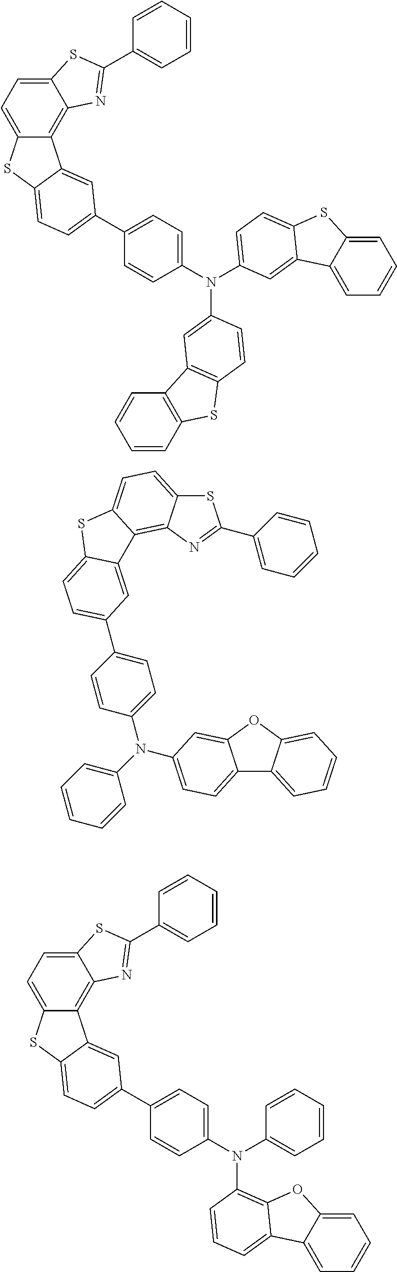

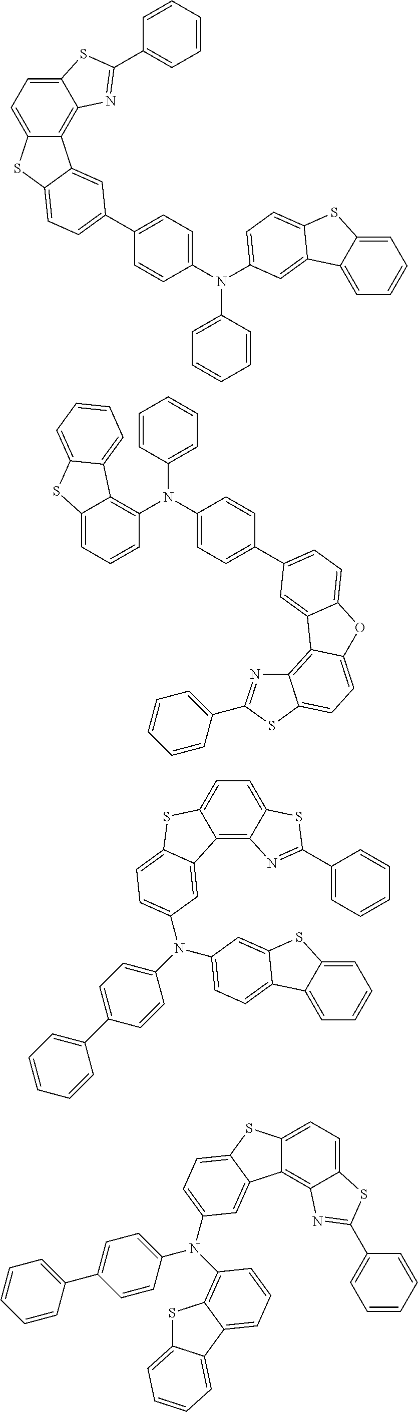

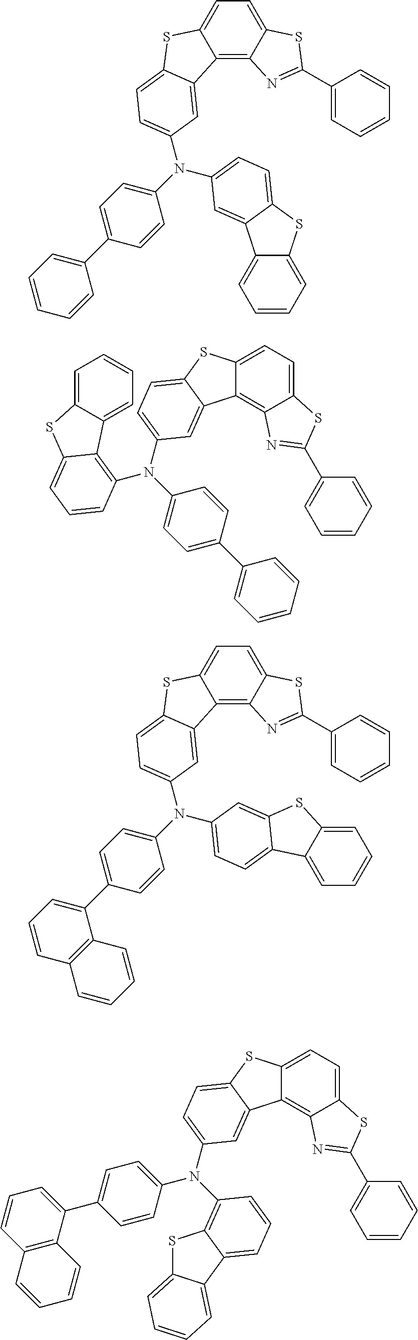

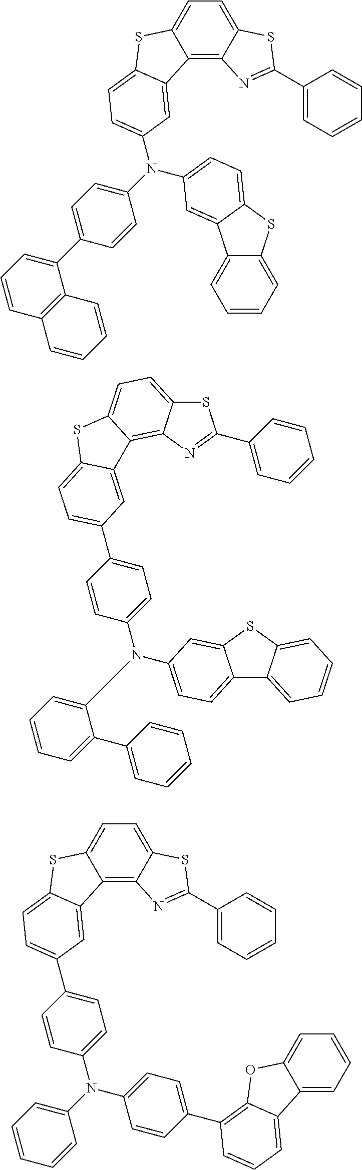

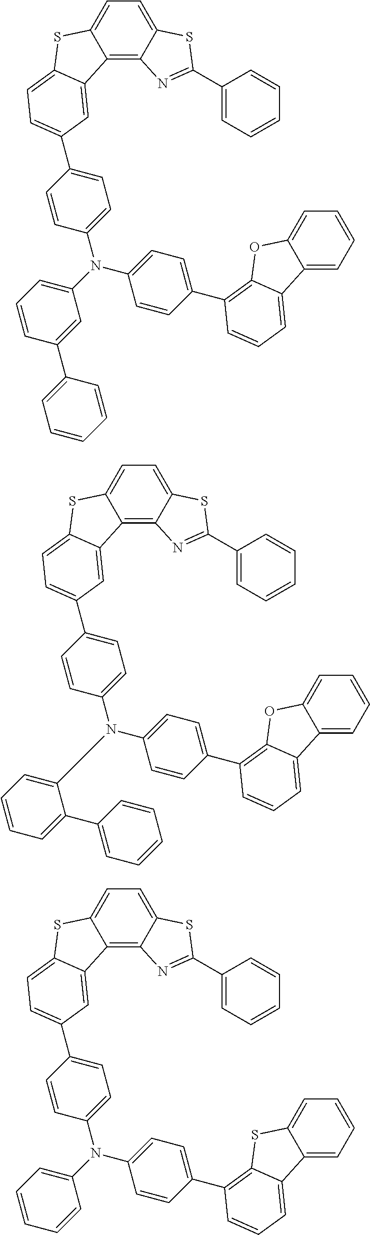

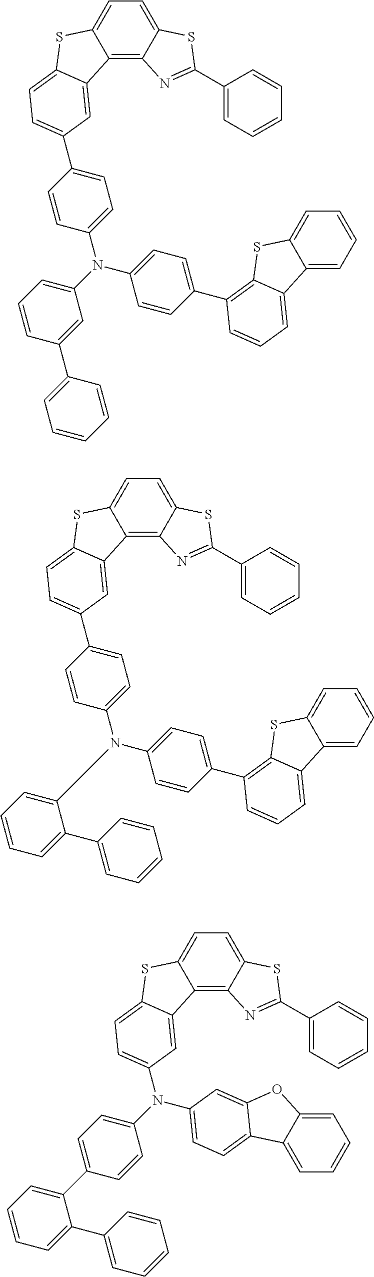 [SMILES: c1ccc(-c2cccc(N(c3ccc(-c4ccc5sc6ccc7sc(-c8ccccc8)nc7c6c5c4)cc3)c3ccc(-c4cccc5c4sc4ccccc45)cc3)c2)cc1 ; c1ccc(-c2nc3c(ccc4sc5ccc(-c6ccc(N(c7ccc(-c8cccc9c8sc8ccccc89)cc7)c7ccccc7-c7ccccc7)cc6)cc5c43)s2)cc1 ; c1ccc(-c2nc3c(ccc4sc5ccc(N(c6ccc(-c7ccccc7-c7ccccc7)cc6)c6ccc7c(c6)oc6ccccc67)cc5c43)s2)cc1]

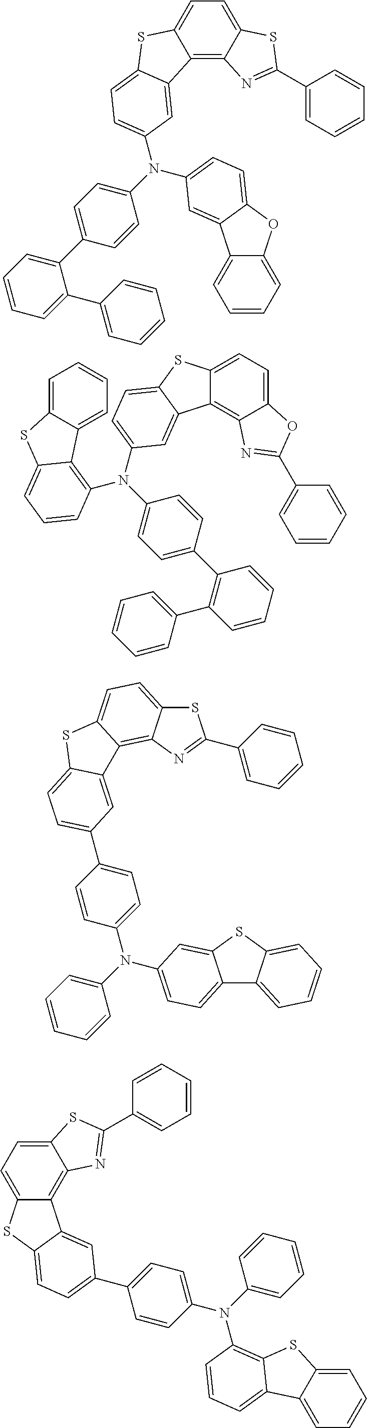

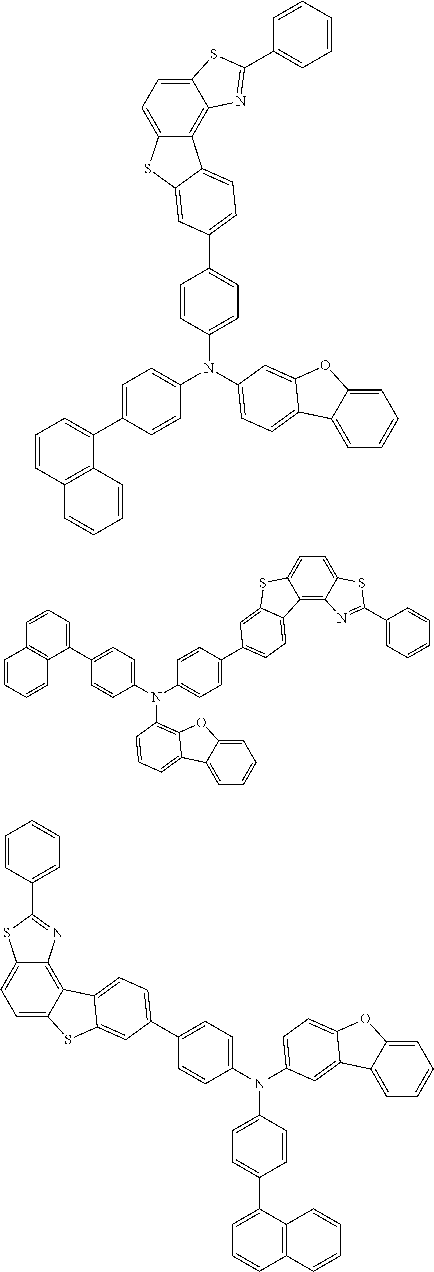 [SMILES: c1ccc(-c2nc3c(ccc4sc5cc(-c6ccc(N(c7ccc(-c8cccc9ccccc89)cc7)c7ccc8c(c7)oc7ccccc78)cc6)ccc5c43)s2)cc1 ; c1ccc(-c2nc3c(ccc4sc5cc(-c6ccc(N(c7ccc(-c8cccc9ccccc89)cc7)c7cccc8c7oc7ccccc78)cc6)ccc5c43)s2)cc1 ; c1ccc(-c2nc3c(ccc4sc5cc(-c6ccc(N(c7ccc(-c8cccc9ccccc89)cc7)c7ccc8oc9ccccc9c8c7)cc6)ccc5c43)s2)cc1]

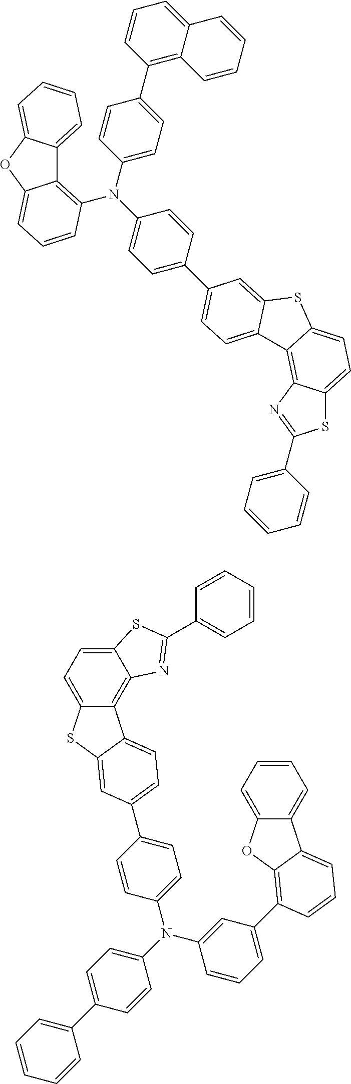 [SMILES: c1ccc(-c2nc3c(ccc4sc5cc(-c6ccc(N(c7ccc(-c8cccc9ccccc89)cc7)c7cccc8oc9ccccc9c78)cc6)ccc5c43)s2)cc1 ; c1ccc(-c2ccc(N(c3ccc(-c4ccc5c(c4)sc4ccc6sc(-c7ccccc7)nc6c45)cc3)c3cccc(-c4cccc5c4oc4ccccc45)c3)cc2)cc1]

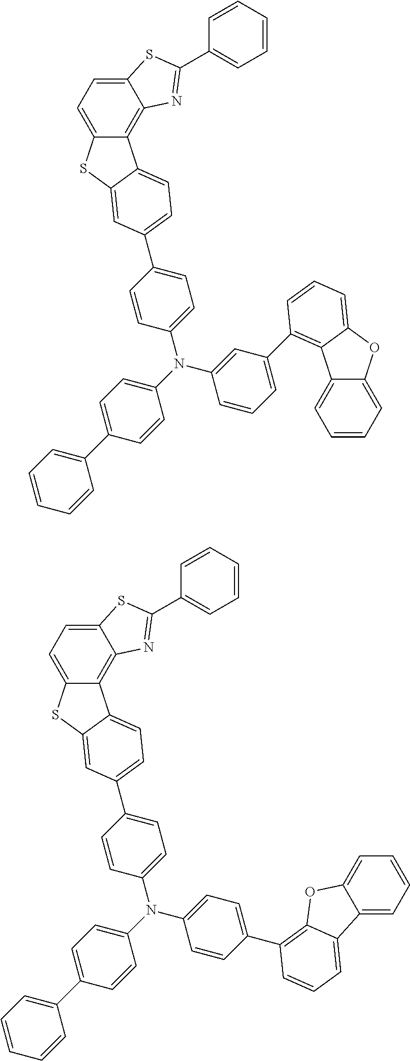 [SMILES: c1ccc(-c2ccc(N(c3ccc(-c4ccc5c(c4)sc4ccc6sc(-c7ccccc7)nc6c45)cc3)c3cccc(-c4cccc5oc6ccccc6c45)c3)cc2)cc1 ; c1ccc(-c2ccc(N(c3ccc(-c4ccc5c(c4)sc4ccc6sc(-c7ccccc7)nc6c45)cc3)c3ccc(-c4cccc5c4oc4ccccc45)cc3)cc2)cc1]

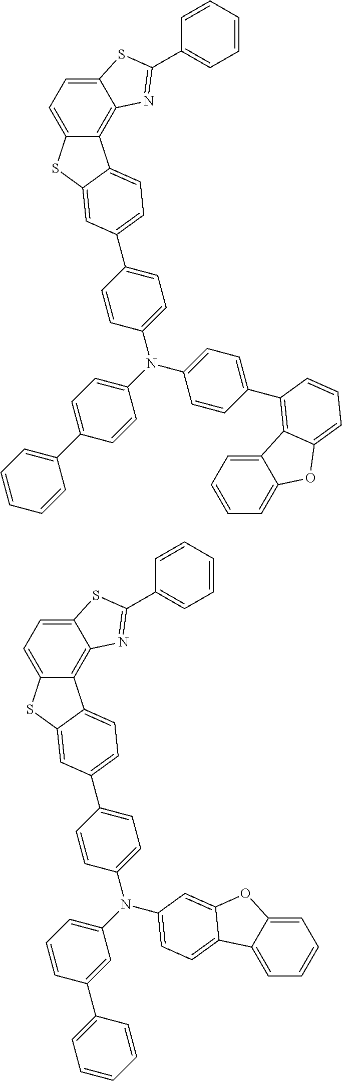 [SMILES: c1ccc(-c2ccc(N(c3ccc(-c4ccc5c(c4)sc4ccc6sc(-c7ccccc7)nc6c45)cc3)c3ccc(-c4cccc5oc6ccccc6c45)cc3)cc2)cc1 ; c1ccc(-c2cccc(N(c3ccc(-c4ccc5c(c4)sc4ccc6sc(-c7ccccc7)nc6c45)cc3)c3ccc4c(c3)oc3ccccc34)c2)cc1]

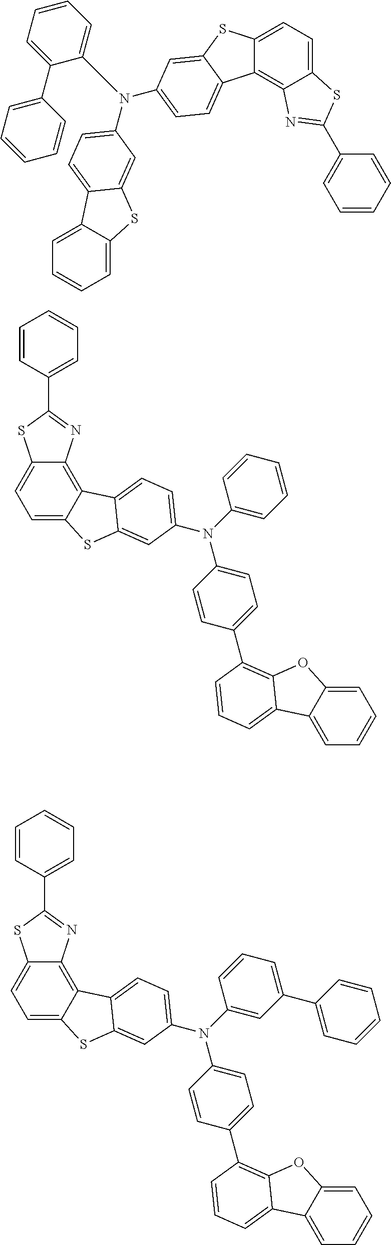

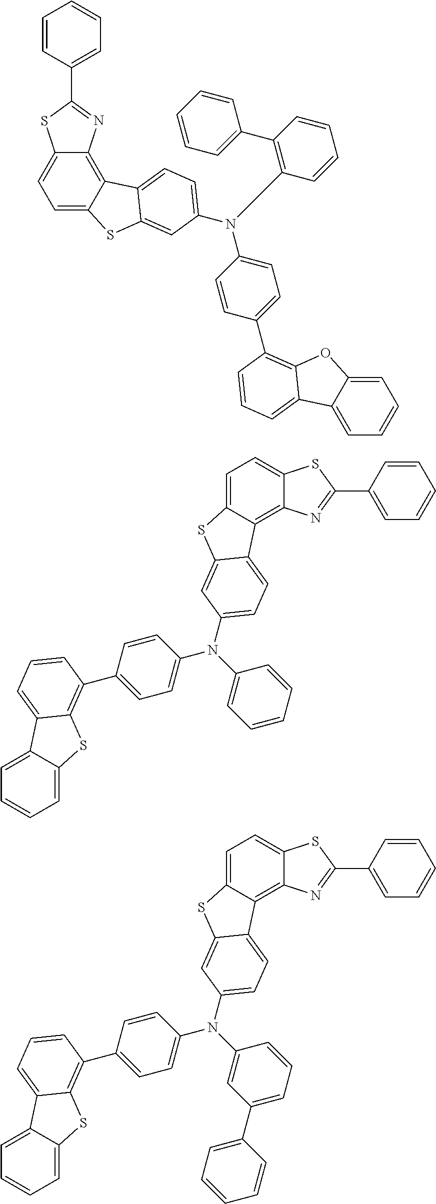

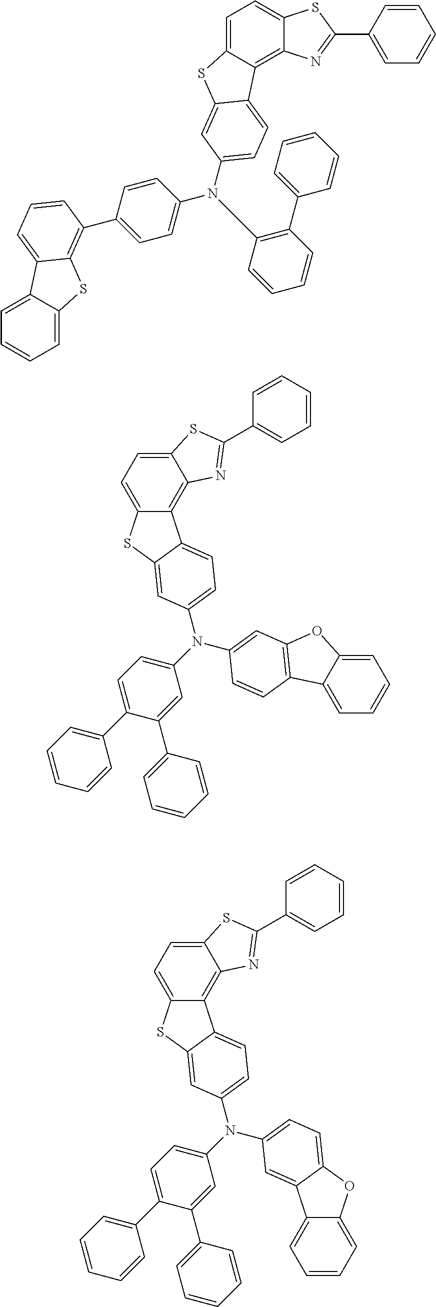 [SMILES: c1ccc(-c2nc3c(ccc4sc5cc(N(c6ccc(-c7cccc8c7sc7ccccc78)cc6)c6ccccc6-c6ccccc6)ccc5c43)s2)cc1 ; c1ccc(-c2nc3c(ccc4sc5cc(N(c6ccc(-c7ccccc7)c(-c7ccccc7)c6)c6ccc7c(c6)oc6ccccc67)ccc5c43)s2)cc1 ; c1ccc(-c2nc3c(ccc4sc5cc(N(c6ccc(-c7ccccc7)c(-c7ccccc7)c6)c6ccc7oc8ccccc8c7c6)ccc5c43)s2)cc1]

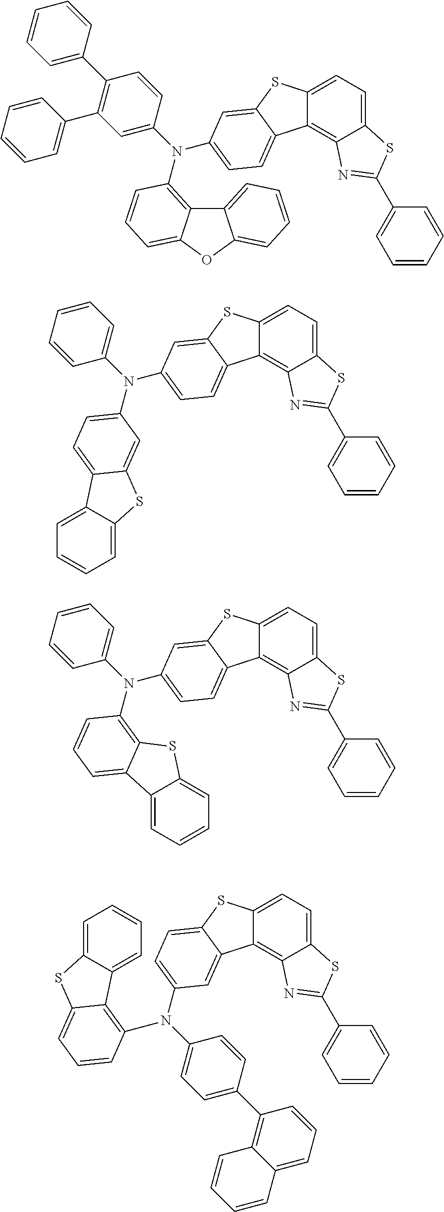

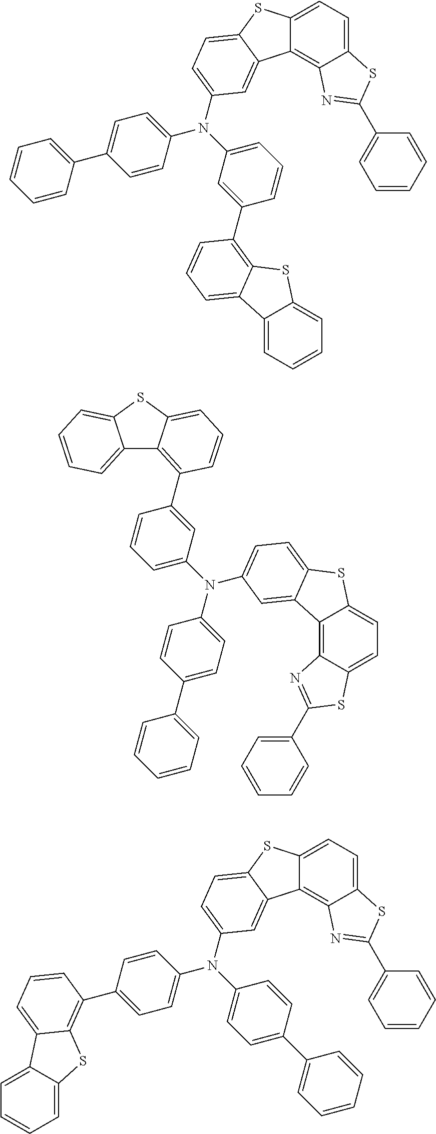 [SMILES: c1ccc(-c2ccc(N(c3cccc(-c4cccc5c4sc4ccccc45)c3)c3ccc4sc5ccc6sc(-c7ccccc7)nc6c5c4c3)cc2)cc1 ; c1ccc(-c2ccc(N(c3cccc(-c4cccc5sc6ccccc6c45)c3)c3ccc4sc5ccc6sc(-c7ccccc7)nc6c5c4c3)cc2)cc1 ; c1ccc(-c2ccc(N(c3ccc(-c4cccc5c4sc4ccccc45)cc3)c3ccc4sc5ccc6sc(-c7ccccc7)nc6c5c4c3)cc2)cc1]

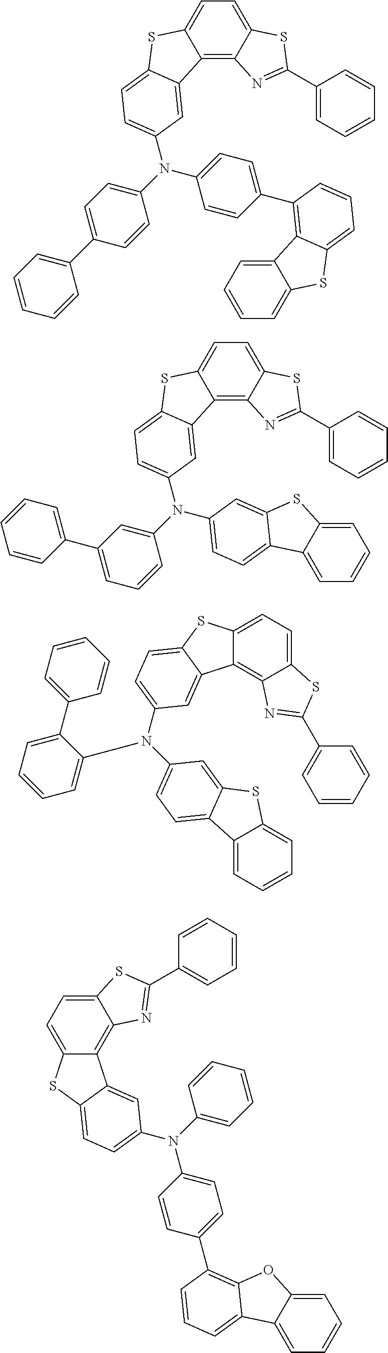

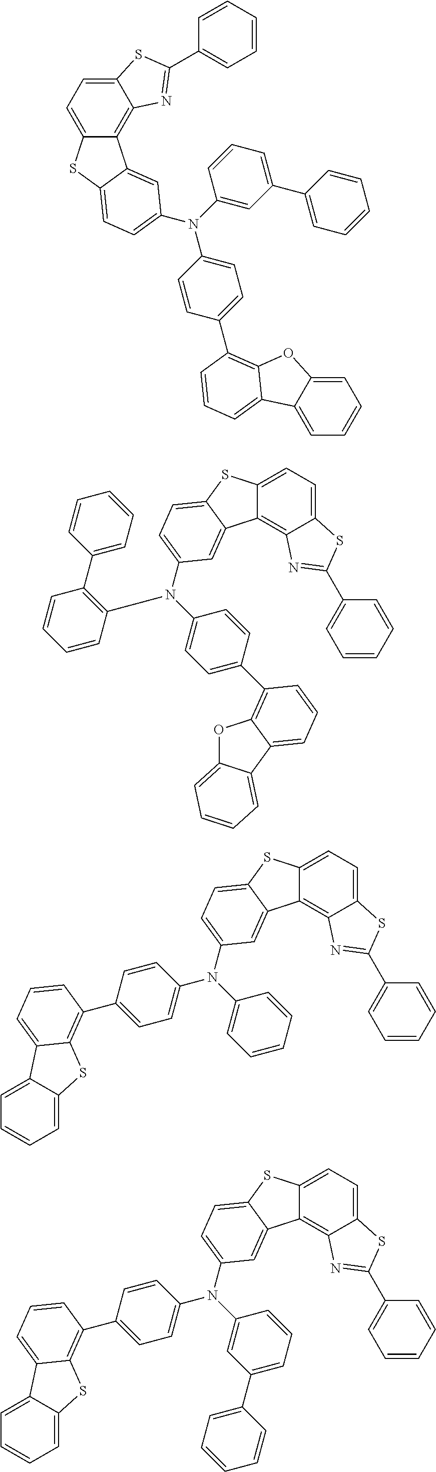

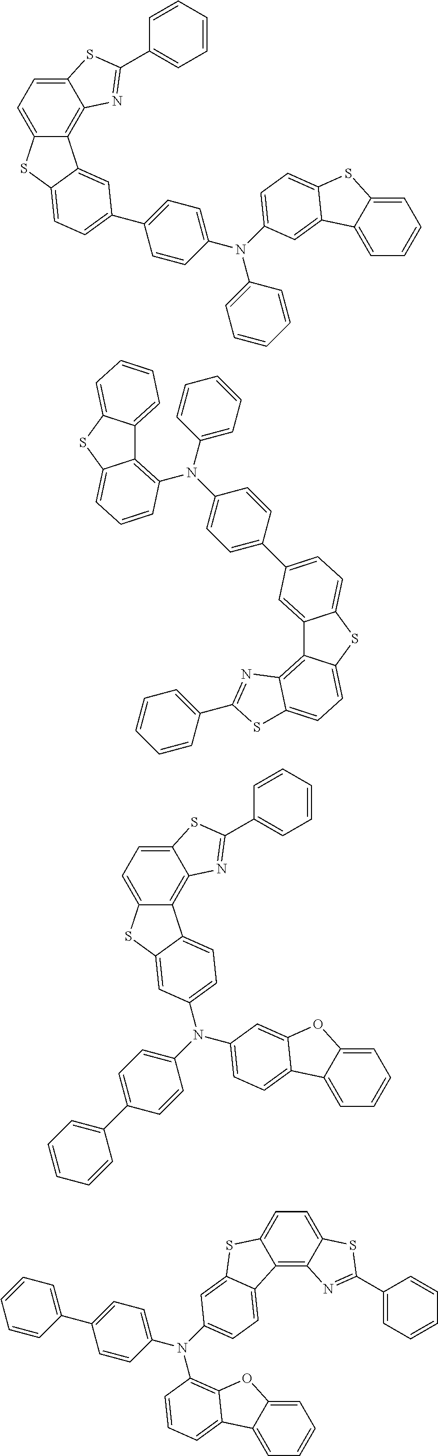

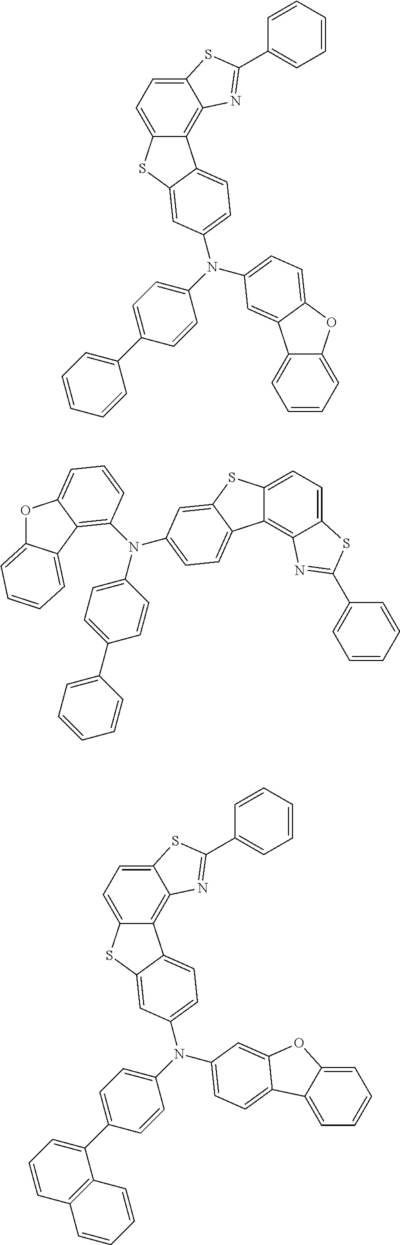 [SMILES: c1ccc(-c2ccc(N(c3ccc4c(c3)sc3ccc5sc(-c6ccccc6)nc5c34)c3ccc4oc5ccccc5c4c3)cc2)cc1 ; c1ccc(-c2ccc(N(c3ccc4c(c3)sc3ccc5sc(-c6ccccc6)nc5c34)c3cccc4oc5ccccc5c34)cc2)cc1 ; c1ccc(-c2nc3c(ccc4sc5cc(N(c6ccc(-c7cccc8ccccc78)cc6)c6ccc7c(c6)oc6ccccc67)ccc5c43)s2)cc1]

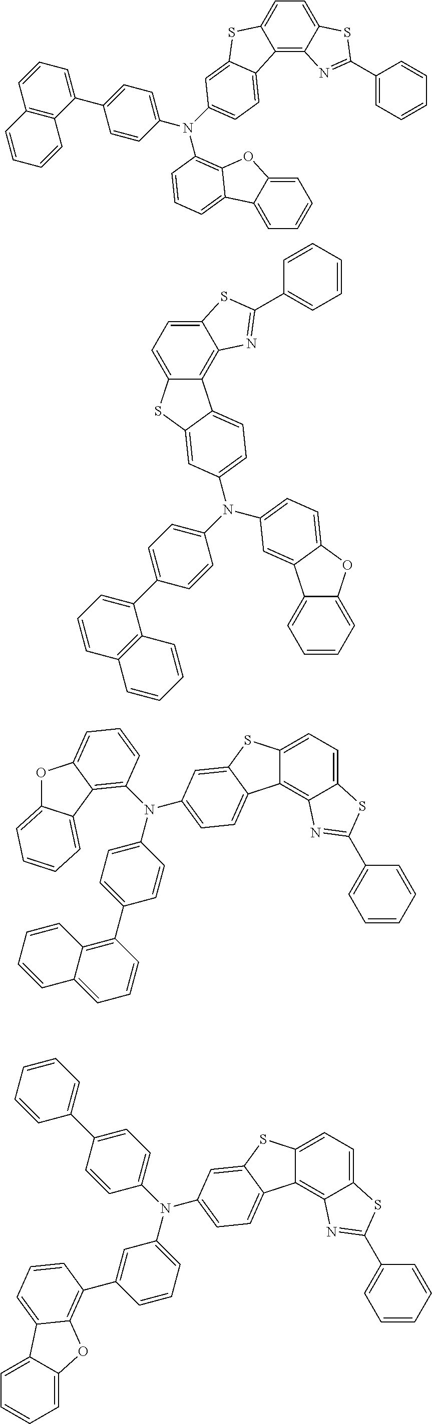

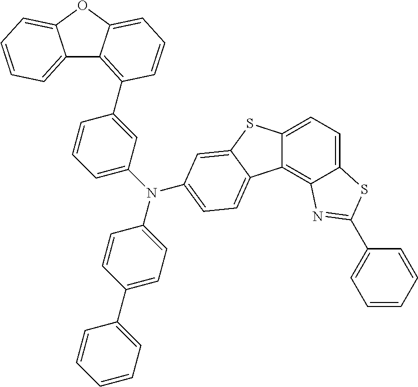 [SMILES: c1ccc(-c2ccc(N(c3cccc(-c4cccc5oc6ccccc6c45)c3)c3ccc4c(c3)sc3ccc5sc(-c6ccccc6)nc5c34)cc2)cc1]

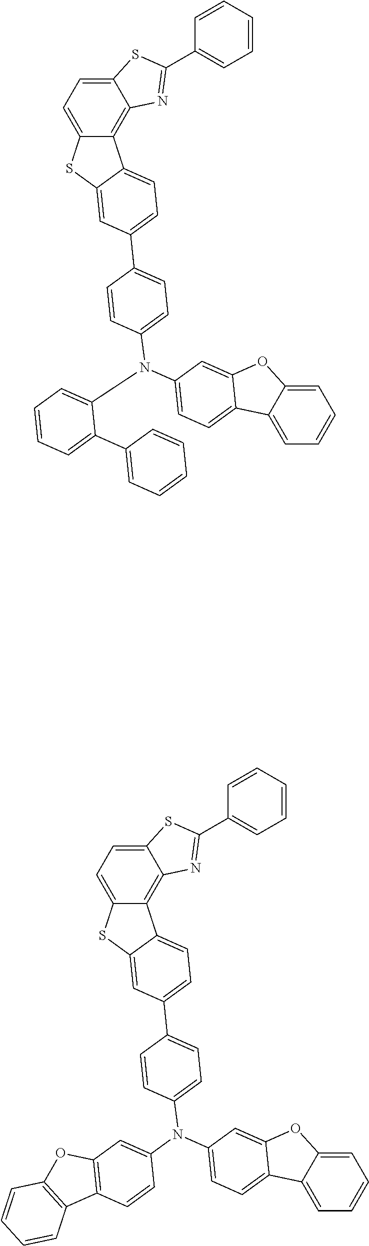 [SMILES: c1ccc(-c2nc3c(ccc4sc5cc(-c6ccc(N(c7ccc8c(c7)oc7ccccc78)c7ccccc7-c7ccccc7)cc6)ccc5c43)s2)cc1 ; c1ccc(-c2nc3c(ccc4sc5cc(-c6ccc(N(c7ccc8c(c7)oc7ccccc78)c7ccc8c(c7)oc7ccccc78)cc6)ccc5c43)s2)cc1]

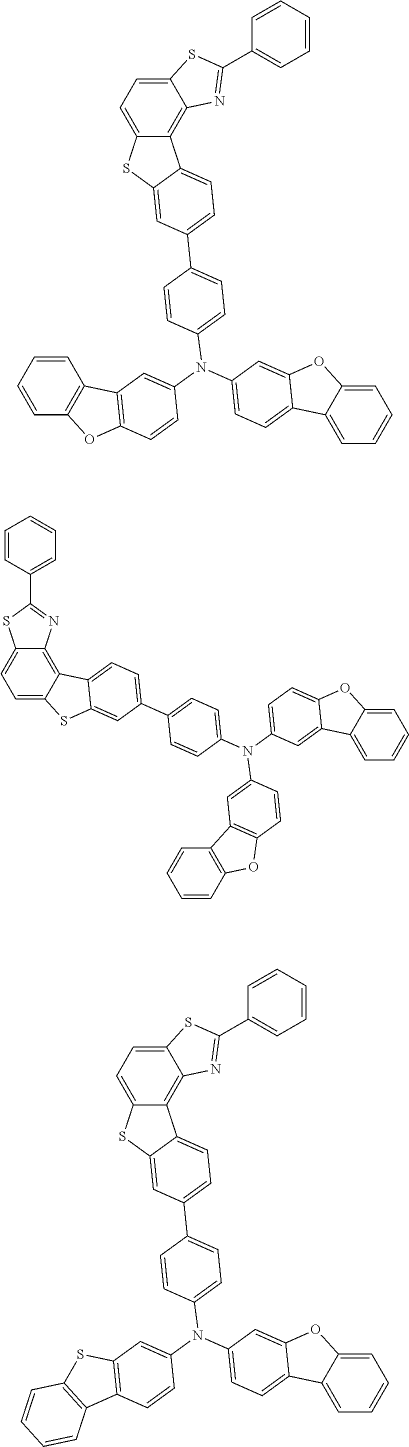 [SMILES: c1ccc(-c2nc3c(ccc4sc5cc(-c6ccc(N(c7ccc8c(c7)oc7ccccc78)c7ccc8oc9ccccc9c8c7)cc6)ccc5c43)s2)cc1 ; c1ccc(-c2nc3c(ccc4sc5cc(-c6ccc(N(c7ccc8oc9ccccc9c8c7)c7ccc8oc9ccccc9c8c7)cc6)ccc5c43)s2)cc1 ; c1ccc(-c2nc3c(ccc4sc5cc(-c6ccc(N(c7ccc8c(c7)oc7ccccc78)c7ccc8c(c7)sc7ccccc78)cc6)ccc5c43)s2)cc1]

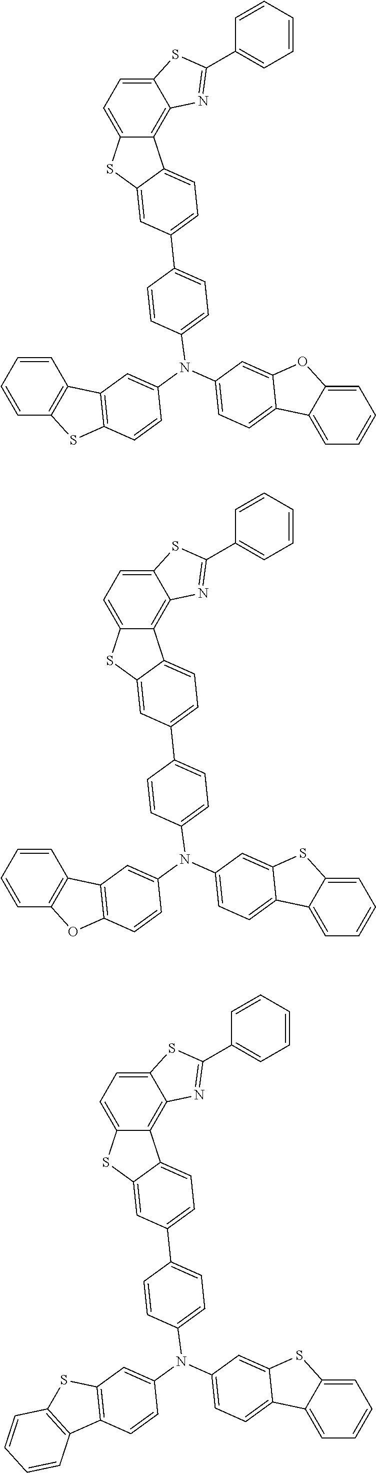 [SMILES: c1ccc(-c2nc3c(ccc4sc5cc(-c6ccc(N(c7ccc8c(c7)oc7ccccc78)c7ccc8sc9ccccc9c8c7)cc6)ccc5c43)s2)cc1 ; c1ccc(-c2nc3c(ccc4sc5cc(-c6ccc(N(c7ccc8c(c7)sc7ccccc78)c7ccc8oc9ccccc9c8c7)cc6)ccc5c43)s2)cc1 ; c1ccc(-c2nc3c(ccc4sc5cc(-c6ccc(N(c7ccc8c(c7)sc7ccccc78)c7ccc8c(c7)sc7ccccc78)cc6)ccc5c43)s2)cc1]

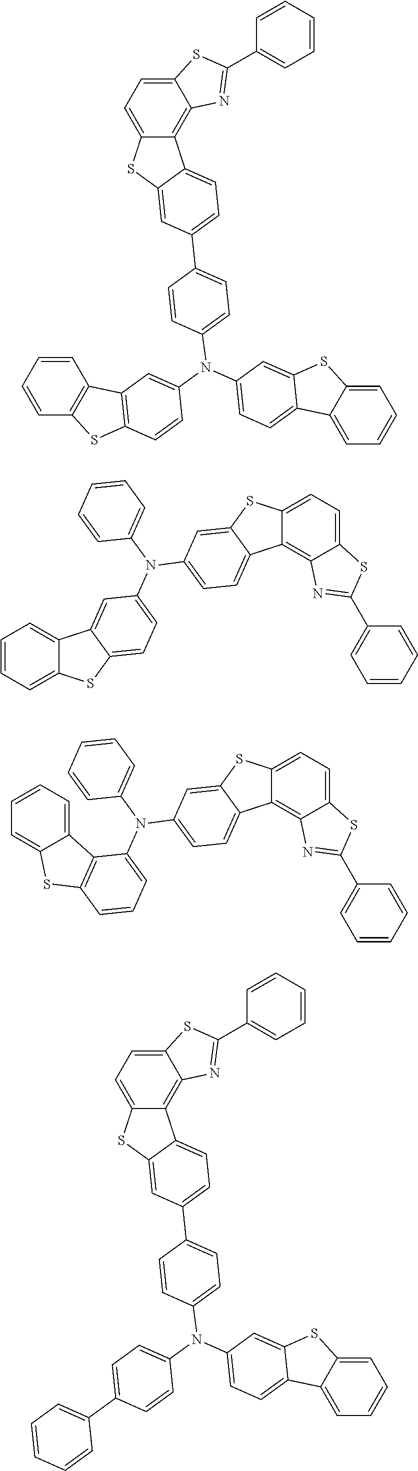

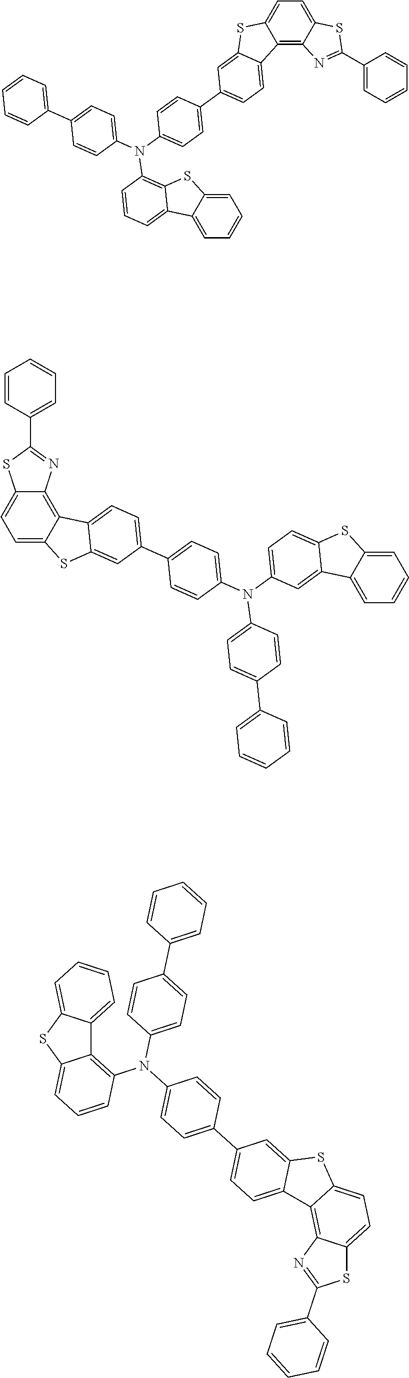 [SMILES: c1ccc(-c2ccc(N(c3ccc(-c4ccc5c(c4)sc4ccc6sc(-c7ccccc7)nc6c45)cc3)c3cccc4c3sc3ccccc34)cc2)cc1 ; c1ccc(-c2ccc(N(c3ccc(-c4ccc5c(c4)sc4ccc6sc(-c7ccccc7)nc6c45)cc3)c3ccc4sc5ccccc5c4c3)cc2)cc1 ; c1ccc(-c2ccc(N(c3ccc(-c4ccc5c(c4)sc4ccc6sc(-c7ccccc7)nc6c45)cc3)c3cccc4sc5ccccc5c34)cc2)cc1]

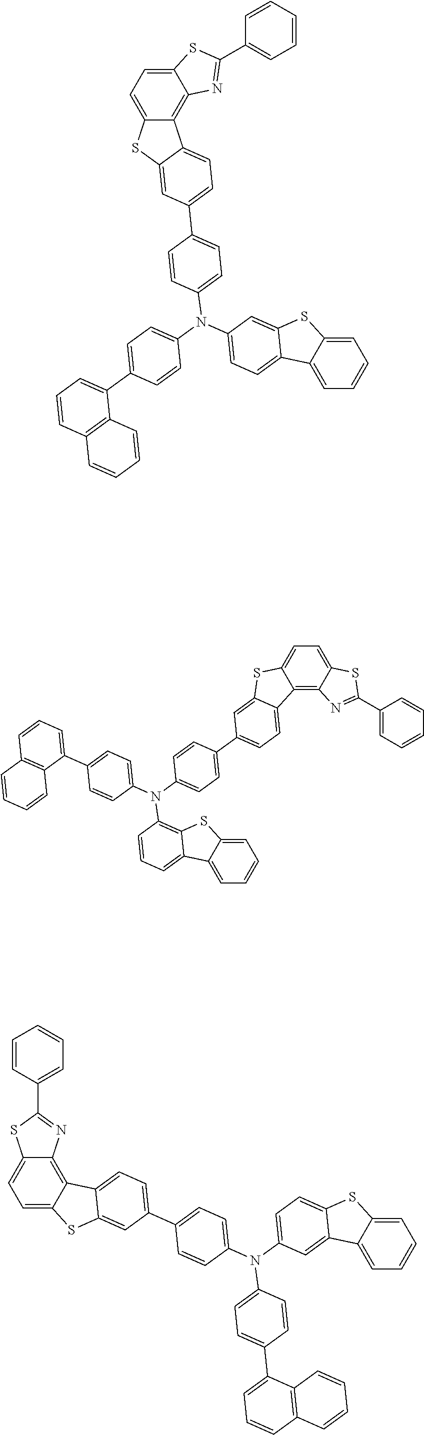 [SMILES: c1ccc(-c2nc3c(ccc4sc5cc(-c6ccc(N(c7ccc(-c8cccc9ccccc89)cc7)c7ccc8c(c7)sc7ccccc78)cc6)ccc5c43)s2)cc1 ; c1ccc(-c2nc3c(ccc4sc5cc(-c6ccc(N(c7ccc(-c8cccc9ccccc89)cc7)c7cccc8c7sc7ccccc78)cc6)ccc5c43)s2)cc1 ; c1ccc(-c2nc3c(ccc4sc5cc(-c6ccc(N(c7ccc(-c8cccc9ccccc89)cc7)c7ccc8sc9ccccc9c8c7)cc6)ccc5c43)s2)cc1]

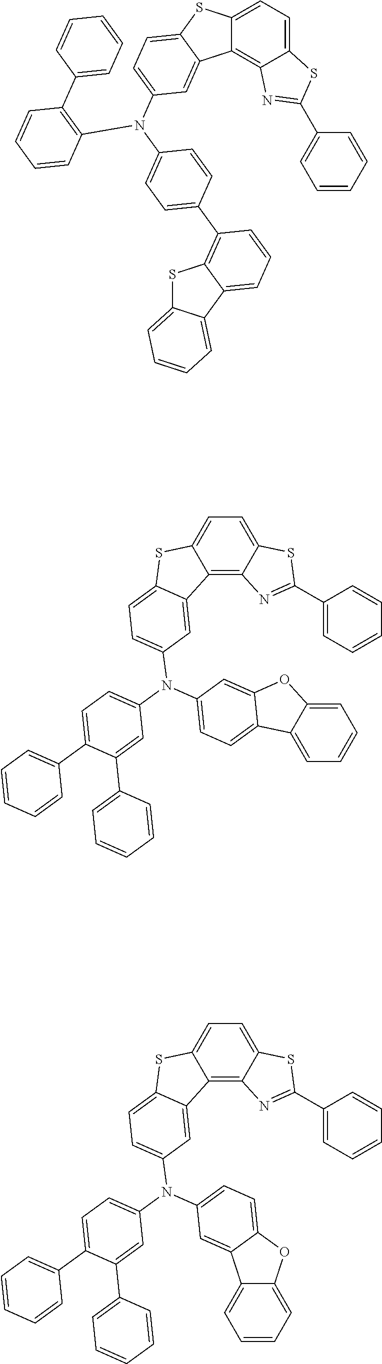 [SMILES: c1ccc(-c2nc3c(ccc4sc5ccc(N(c6ccc(-c7cccc8c7sc7ccccc78)cc6)c6ccccc6-c6ccccc6)cc5c43)s2)cc1 ; c1ccc(-c2nc3c(ccc4sc5ccc(N(c6ccc(-c7ccccc7)c(-c7ccccc7)c6)c6ccc7c(c6)oc6ccccc67)cc5c43)s2)cc1 ; c1ccc(-c2nc3c(ccc4sc5ccc(N(c6ccc(-c7ccccc7)c(-c7ccccc7)c6)c6ccc7oc8ccccc8c7c6)cc5c43)s2)cc1]

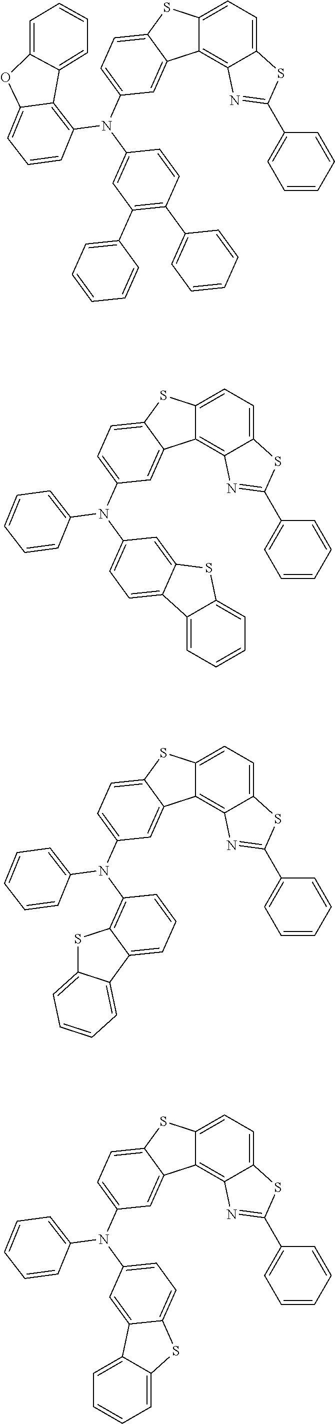 [SMILES: c1ccc(-c2nc3c(ccc4sc5ccc(N(c6ccc(-c7ccccc7)c(-c7ccccc7)c6)c6cccc7oc8ccccc8c67)cc5c43)s2)cc1 ; c1ccc(-c2nc3c(ccc4sc5ccc(N(c6ccccc6)c6ccc7c(c6)sc6ccccc67)cc5c43)s2)cc1 ; c1ccc(-c2nc3c(ccc4sc5ccc(N(c6ccccc6)c6cccc7c6sc6ccccc67)cc5c43)s2)cc1 ; c1ccc(-c2nc3c(ccc4sc5ccc(N(c6ccccc6)c6ccc7sc8ccccc8c7c6)cc5c43)s2)cc1]

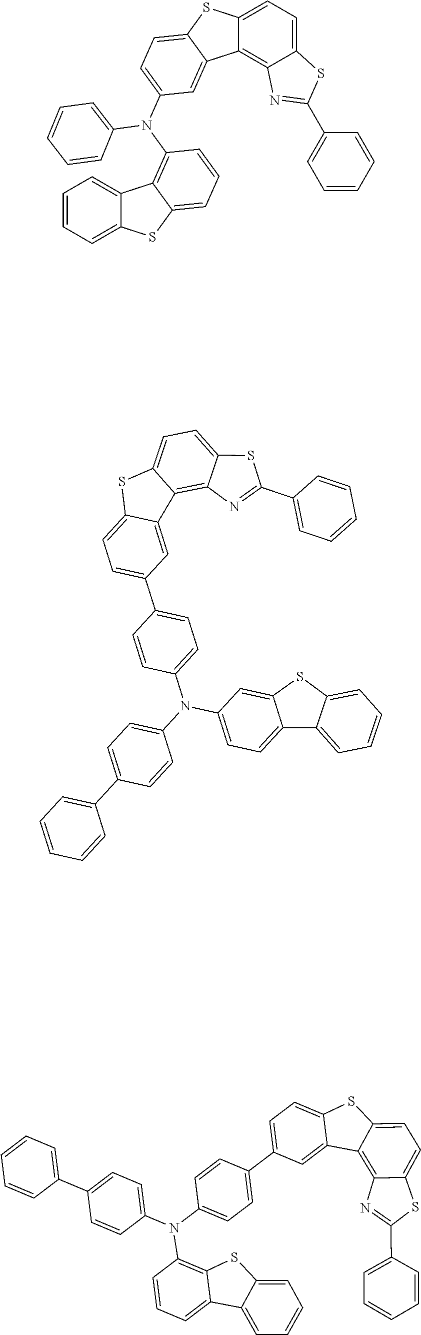 [SMILES: c1ccc(-c2nc3c(ccc4sc5ccc(N(c6ccccc6)c6cccc7sc8ccccc8c67)cc5c43)s2)cc1 ; c1ccc(-c2ccc(N(c3ccc(-c4ccc5sc6ccc7sc(-c8ccccc8)nc7c6c5c4)cc3)c3ccc4c(c3)sc3ccccc34)cc2)cc1 ; c1ccc(-c2ccc(N(c3ccc(-c4ccc5sc6ccc7sc(-c8ccccc8)nc7c6c5c4)cc3)c3cccc4c3sc3ccccc34)cc2)cc1]

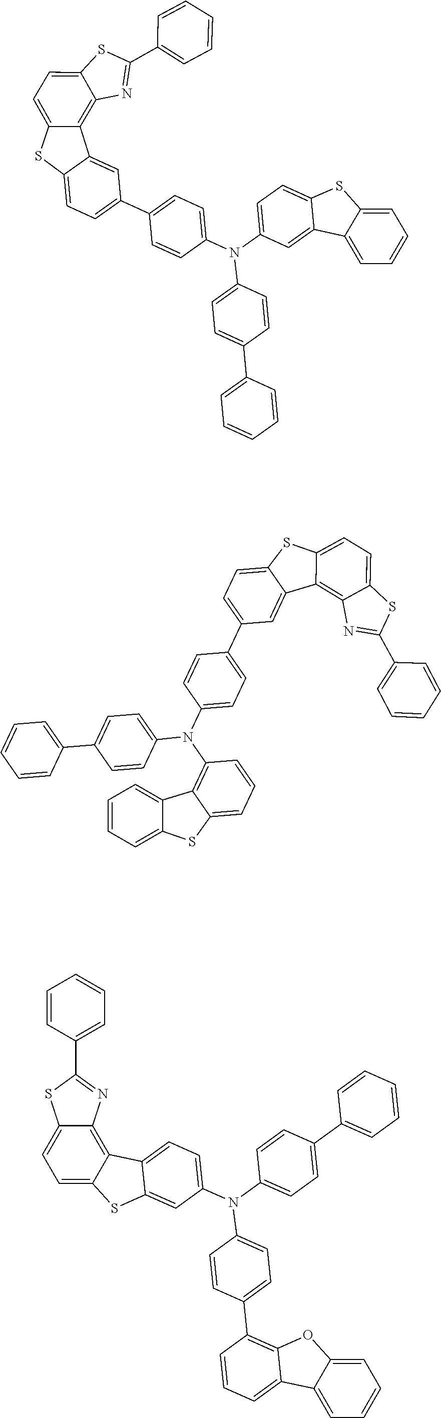 [SMILES: c1ccc(-c2ccc(N(c3ccc(-c4ccc5sc6ccc7sc(-c8ccccc8)nc7c6c5c4)cc3)c3ccc4sc5ccccc5c4c3)cc2)cc1 ; c1ccc(-c2ccc(N(c3ccc(-c4ccc5sc6ccc7sc(-c8ccccc8)nc7c6c5c4)cc3)c3cccc4sc5ccccc5c34)cc2)cc1 ; c1ccc(-c2ccc(N(c3ccc(-c4cccc5c4oc4ccccc45)cc3)c3ccc4c(c3)sc3ccc5sc(-c6ccccc6)nc5c34)cc2)cc1]

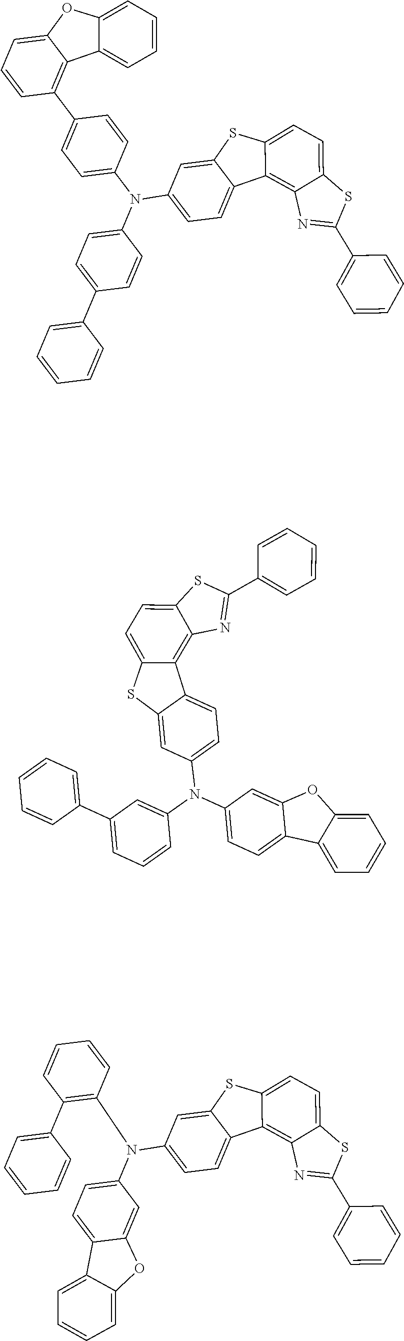 [SMILES: c1ccc(-c2ccc(N(c3ccc(-c4cccc5oc6ccccc6c45)cc3)c3ccc4c(c3)sc3ccc5sc(-c6ccccc6)nc5c34)cc2)cc1 ; c1ccc(-c2cccc(N(c3ccc4c(c3)oc3ccccc34)c3ccc4c(c3)sc3ccc5sc(-c6ccccc6)nc5c34)c2)cc1 ; c1ccc(-c2nc3c(ccc4sc5cc(N(c6ccc7c(c6)oc6ccccc67)c6ccccc6-c6ccccc6)ccc5c43)s2)cc1]

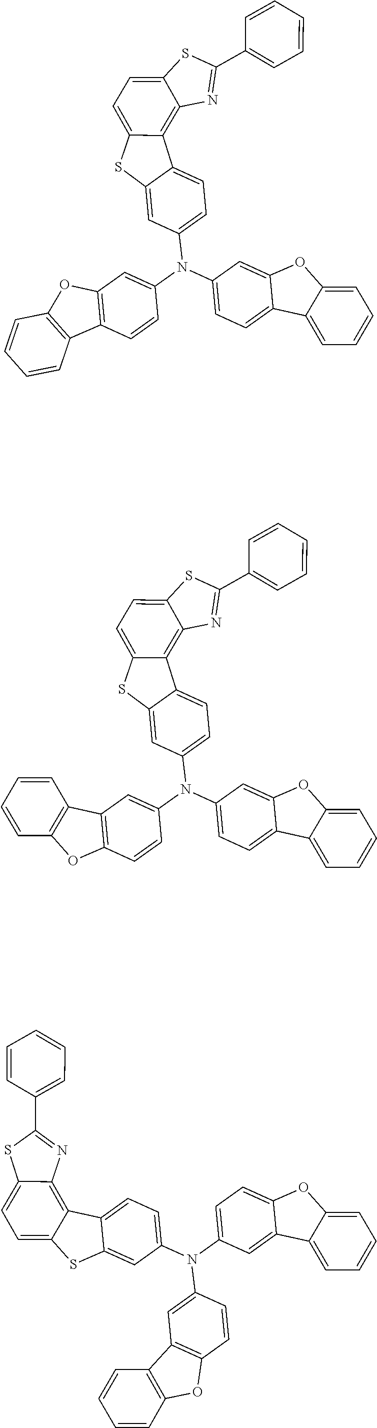 [SMILES: c1ccc(-c2nc3c(ccc4sc5cc(N(c6ccc7c(c6)oc6ccccc67)c6ccc7c(c6)oc6ccccc67)ccc5c43)s2)cc1 ; c1ccc(-c2nc3c(ccc4sc5cc(N(c6ccc7c(c6)oc6ccccc67)c6ccc7oc8ccccc8c7c6)ccc5c43)s2)cc1 ; c1ccc(-c2nc3c(ccc4sc5cc(N(c6ccc7oc8ccccc8c7c6)c6ccc7oc8ccccc8c7c6)ccc5c43)s2)cc1]

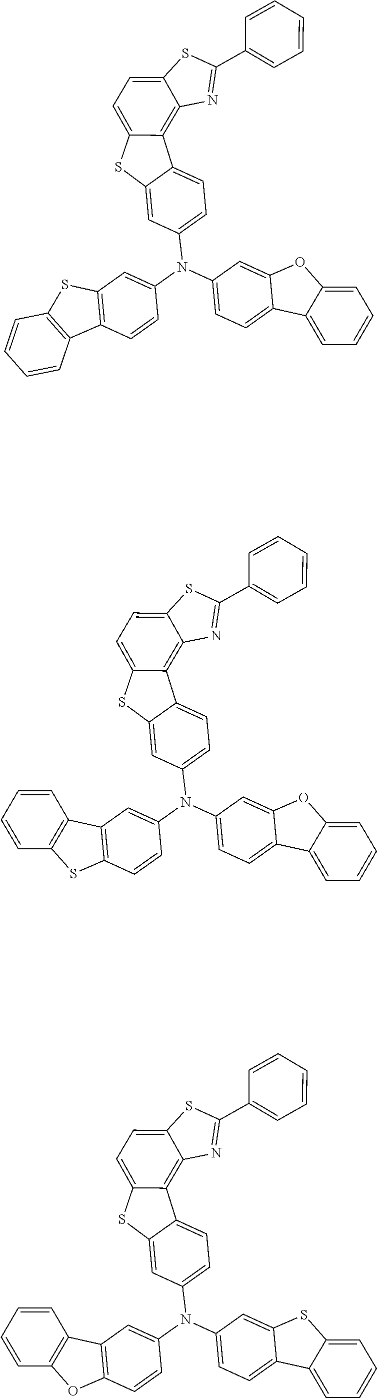 [SMILES: c1ccc(-c2nc3c(ccc4sc5cc(N(c6ccc7c(c6)oc6ccccc67)c6ccc7c(c6)sc6ccccc67)ccc5c43)s2)cc1 ; c1ccc(-c2nc3c(ccc4sc5cc(N(c6ccc7c(c6)oc6ccccc67)c6ccc7sc8ccccc8c7c6)ccc5c43)s2)cc1 ; c1ccc(-c2nc3c(ccc4sc5cc(N(c6ccc7c(c6)sc6ccccc67)c6ccc7oc8ccccc8c7c6)ccc5c43)s2)cc1]

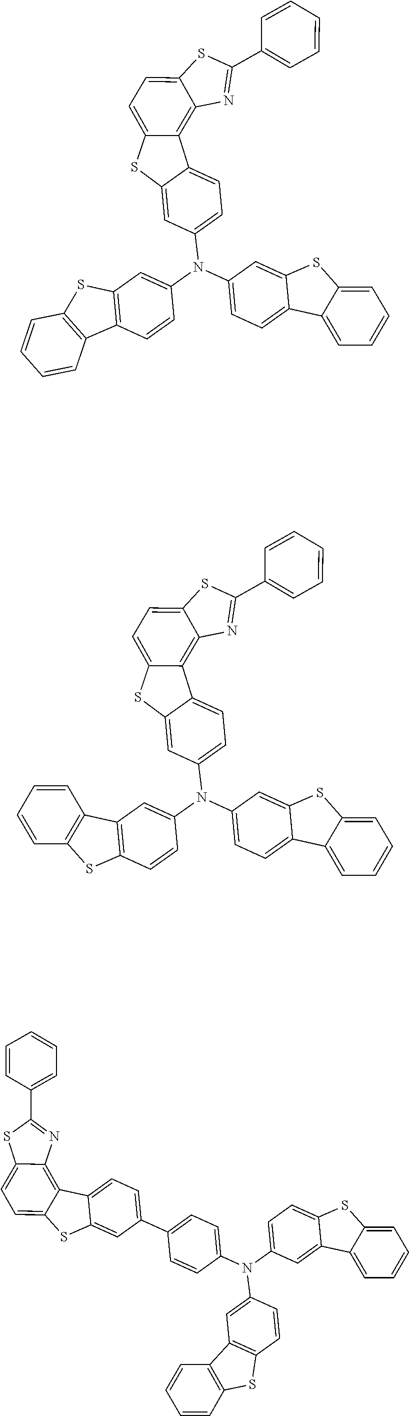 [SMILES: c1ccc(-c2nc3c(ccc4sc5cc(N(c6ccc7c(c6)sc6ccccc67)c6ccc7c(c6)sc6ccccc67)ccc5c43)s2)cc1 ; c1ccc(-c2nc3c(ccc4sc5cc(N(c6ccc7c(c6)sc6ccccc67)c6ccc7sc8ccccc8c7c6)ccc5c43)s2)cc1 ; c1ccc(-c2nc3c(ccc4sc5cc(-c6ccc(N(c7ccc8sc9ccccc9c8c7)c7ccc8sc9ccccc9c8c7)cc6)ccc5c43)s2)cc1]

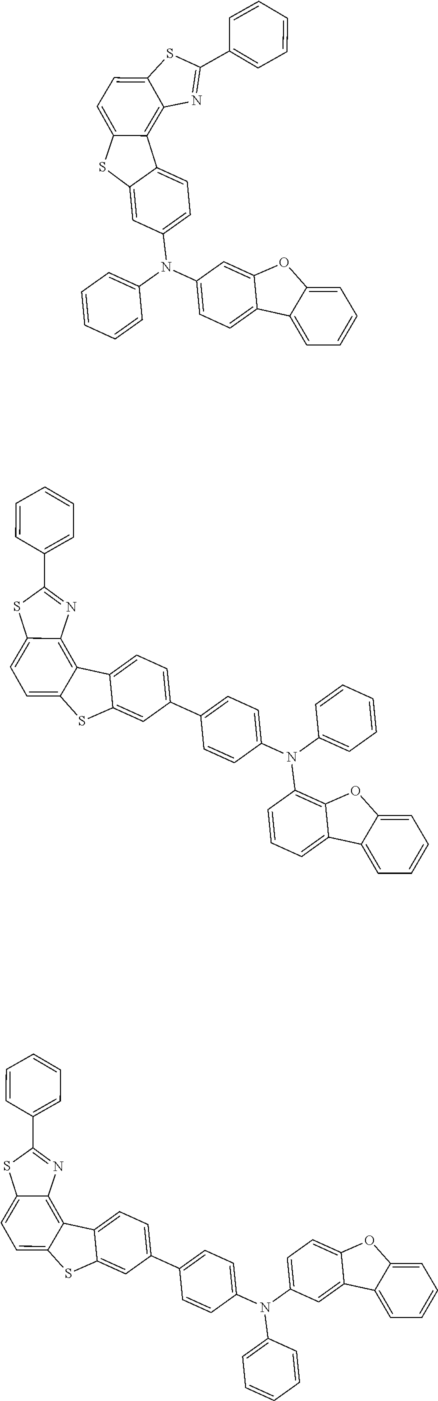 [SMILES: c1ccc(-c2nc3c(ccc4sc5cc(N(c6ccccc6)c6ccc7c(c6)oc6ccccc67)ccc5c43)s2)cc1 ; c1ccc(-c2nc3c(ccc4sc5cc(-c6ccc(N(c7ccccc7)c7cccc8c7oc7ccccc78)cc6)ccc5c43)s2)cc1 ; c1ccc(-c2nc3c(ccc4sc5cc(-c6ccc(N(c7ccccc7)c7ccc8oc9ccccc9c8c7)cc6)ccc5c43)s2)cc1]

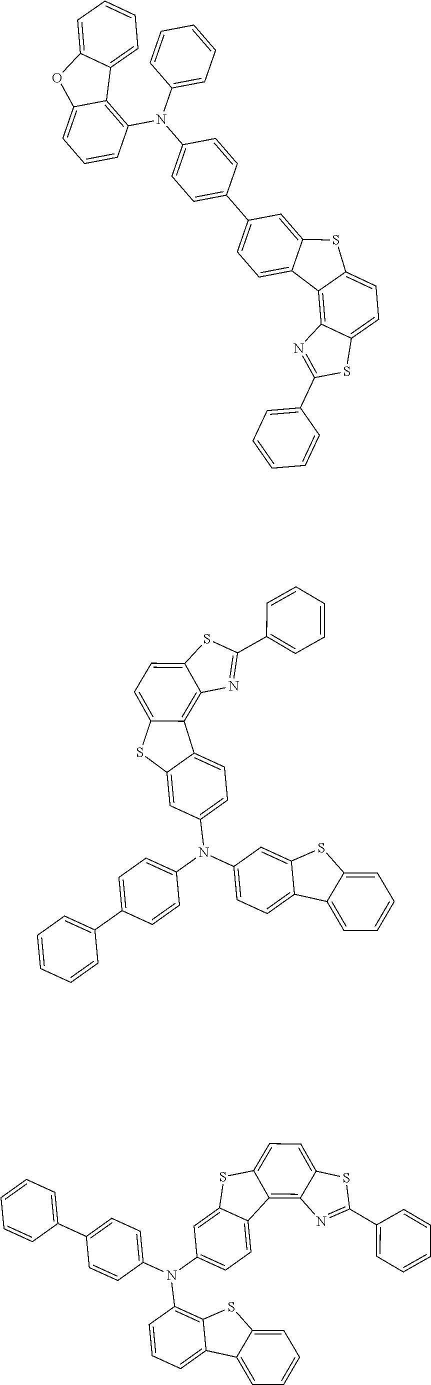 [SMILES: c1ccc(-c2nc3c(ccc4sc5cc(-c6ccc(N(c7ccccc7)c7cccc8oc9ccccc9c78)cc6)ccc5c43)s2)cc1 ; c1ccc(-c2ccc(N(c3ccc4c(c3)sc3ccccc34)c3ccc4c(c3)sc3ccc5sc(-c6ccccc6)nc5c34)cc2)cc1 ; c1ccc(-c2ccc(N(c3ccc4c(c3)sc3ccc5sc(-c6ccccc6)nc5c34)c3cccc4c3sc3ccccc34)cc2)cc1]

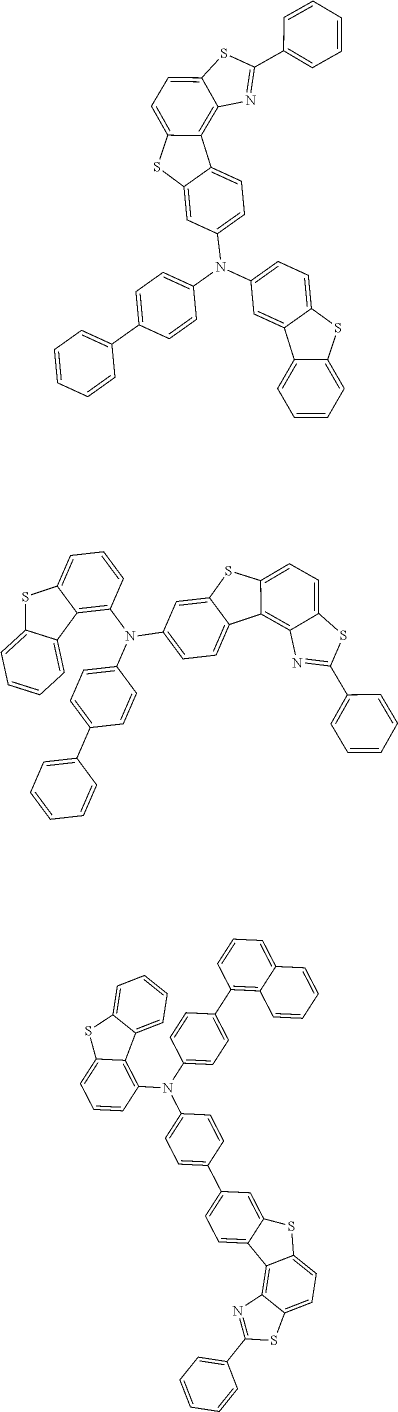 [SMILES: c1ccc(-c2ccc(N(c3ccc4c(c3)sc3ccc5sc(-c6ccccc6)nc5c34)c3ccc4sc5ccccc5c4c3)cc2)cc1 ; c1ccc(-c2ccc(N(c3ccc4c(c3)sc3ccc5sc(-c6ccccc6)nc5c34)c3cccc4sc5ccccc5c34)cc2)cc1 ; c1ccc(-c2nc3c(ccc4sc5cc(-c6ccc(N(c7ccc(-c8cccc9ccccc89)cc7)c7cccc8sc9ccccc9c78)cc6)ccc5c43)s2)cc1]

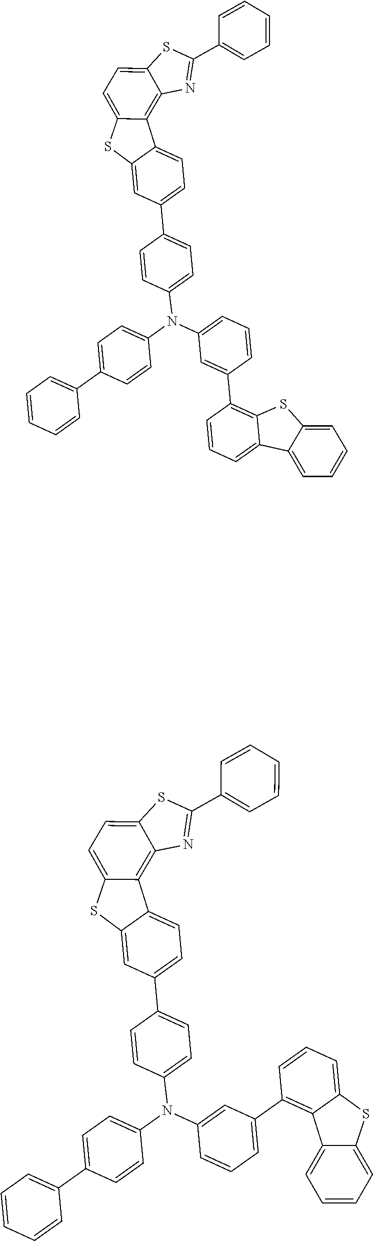 [SMILES: c1ccc(-c2ccc(N(c3ccc(-c4ccc5c(c4)sc4ccc6sc(-c7ccccc7)nc6c45)cc3)c3cccc(-c4cccc5c4sc4ccccc45)c3)cc2)cc1 ; c1ccc(-c2ccc(N(c3ccc(-c4ccc5c(c4)sc4ccc6sc(-c7ccccc7)nc6c45)cc3)c3cccc(-c4cccc5sc6ccccc6c45)c3)cc2)cc1]

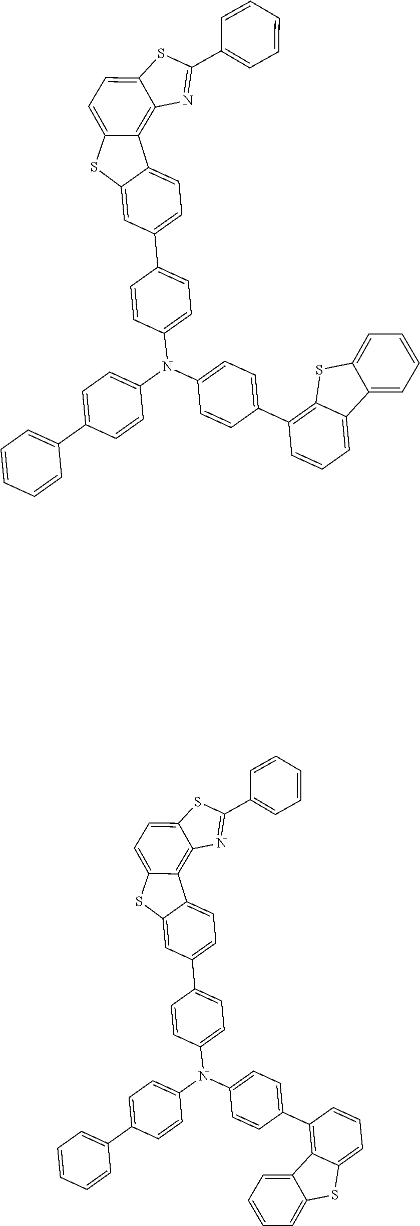 [SMILES: c1ccc(-c2ccc(N(c3ccc(-c4ccc5c(c4)sc4ccc6sc(-c7ccccc7)nc6c45)cc3)c3ccc(-c4cccc5c4sc4ccccc45)cc3)cc2)cc1 ; c1ccc(-c2ccc(N(c3ccc(-c4ccc5c(c4)sc4ccc6sc(-c7ccccc7)nc6c45)cc3)c3ccc(-c4cccc5sc6ccccc6c45)cc3)cc2)cc1]

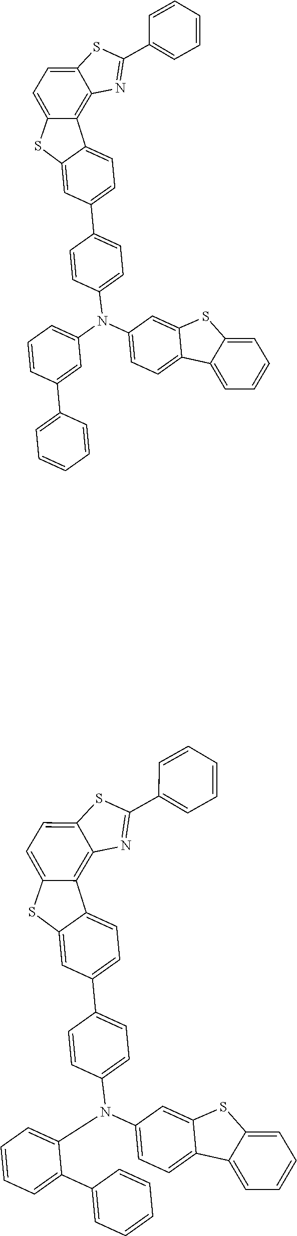 [SMILES: c1ccc(-c2cccc(N(c3ccc(-c4ccc5c(c4)sc4ccc6sc(-c7ccccc7)nc6c45)cc3)c3ccc4c(c3)sc3ccccc34)c2)cc1 ; c1ccc(-c2nc3c(ccc4sc5cc(-c6ccc(N(c7ccc8c(c7)sc7ccccc78)c7ccccc7-c7ccccc7)cc6)ccc5c43)s2)cc1]

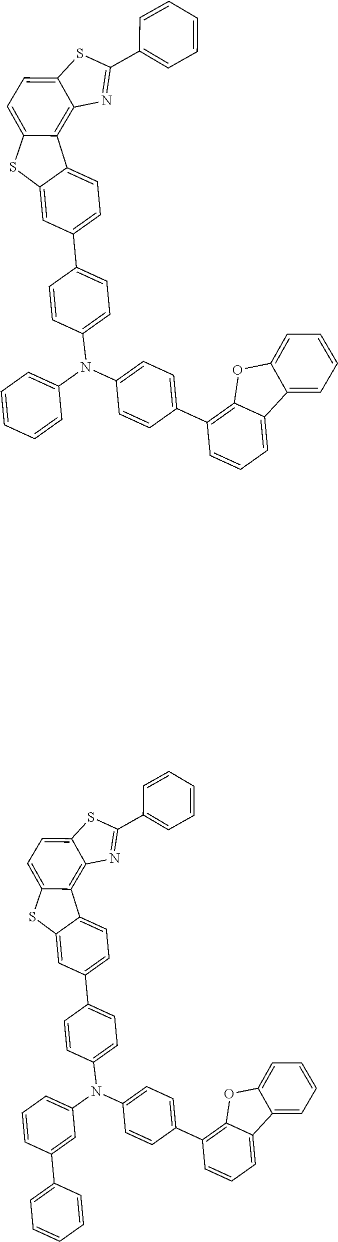 [SMILES: c1ccc(-c2nc3c(ccc4sc5cc(-c6ccc(N(c7ccccc7)c7ccc(-c8cccc9c8oc8ccccc89)cc7)cc6)ccc5c43)s2)cc1 ; c1ccc(-c2cccc(N(c3ccc(-c4ccc5c(c4)sc4ccc6sc(-c7ccccc7)nc6c45)cc3)c3ccc(-c4cccc5c4oc4ccccc45)cc3)c2)cc1]

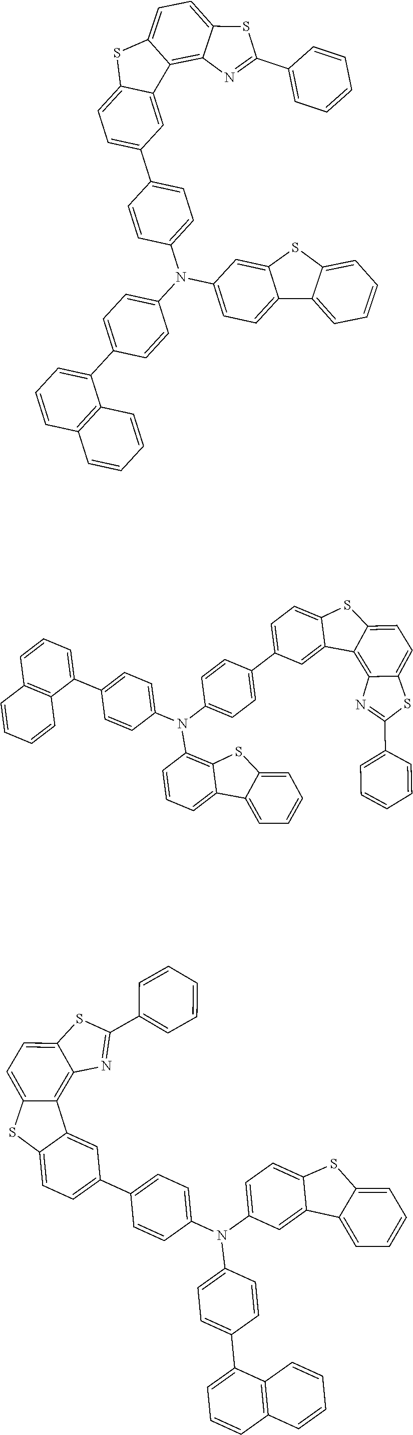 [SMILES: c1ccc(-c2nc3c(ccc4sc5ccc(-c6ccc(N(c7ccc(-c8cccc9ccccc89)cc7)c7ccc8c(c7)sc7ccccc78)cc6)cc5c43)s2)cc1 ; c1ccc(-c2nc3c(ccc4sc5ccc(-c6ccc(N(c7ccc(-c8cccc9ccccc89)cc7)c7cccc8c7sc7ccccc78)cc6)cc5c43)s2)cc1 ; c1ccc(-c2nc3c(ccc4sc5ccc(-c6ccc(N(c7ccc(-c8cccc9ccccc89)cc7)c7ccc8sc9ccccc9c8c7)cc6)cc5c43)s2)cc1]

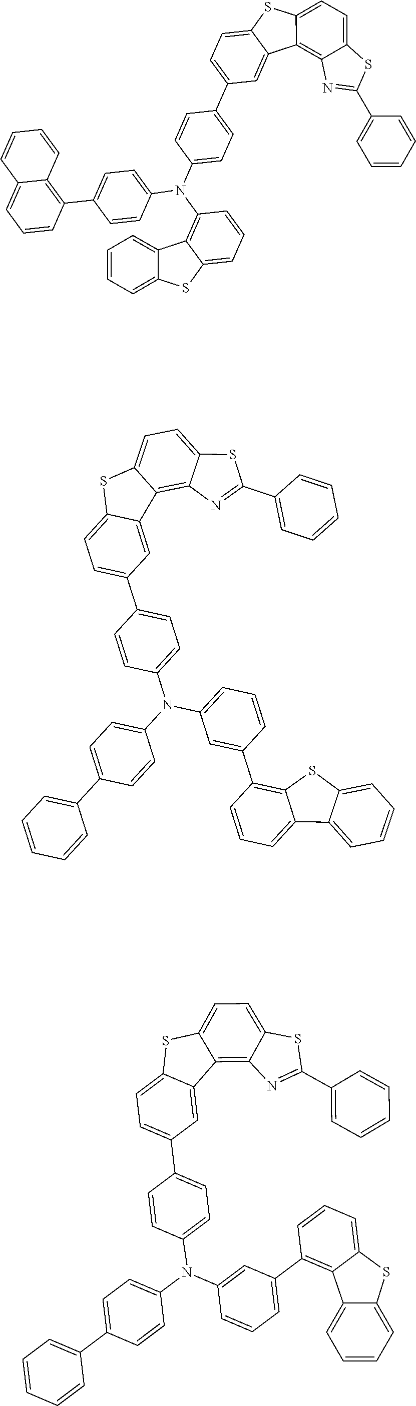 [SMILES: c1ccc(-c2nc3c(ccc4sc5ccc(-c6ccc(N(c7ccc(-c8cccc9ccccc89)cc7)c7cccc8sc9ccccc9c78)cc6)cc5c43)s2)cc1 ; c1ccc(-c2ccc(N(c3ccc(-c4ccc5sc6ccc7sc(-c8ccccc8)nc7c6c5c4)cc3)c3cccc(-c4cccc5c4sc4ccccc45)c3)cc2)cc1 ; c1ccc(-c2ccc(N(c3ccc(-c4ccc5sc6ccc7sc(-c8ccccc8)nc7c6c5c4)cc3)c3cccc(-c4cccc5sc6ccccc6c45)c3)cc2)cc1]

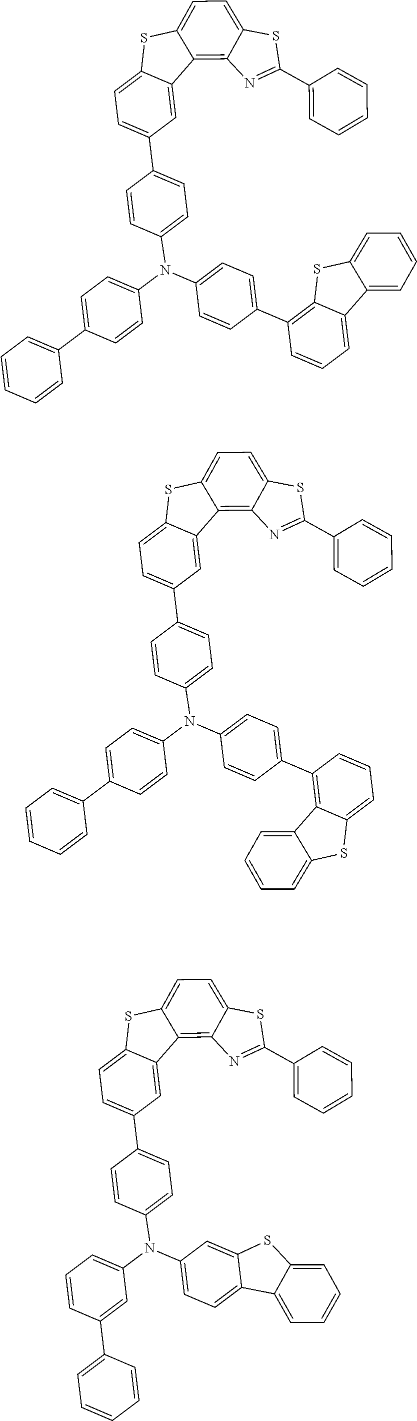 [SMILES: c1ccc(-c2ccc(N(c3ccc(-c4ccc5sc6ccc7sc(-c8ccccc8)nc7c6c5c4)cc3)c3ccc(-c4cccc5c4sc4ccccc45)cc3)cc2)cc1 ; c1ccc(-c2ccc(N(c3ccc(-c4ccc5sc6ccc7sc(-c8ccccc8)nc7c6c5c4)cc3)c3ccc(-c4cccc5sc6ccccc6c45)cc3)cc2)cc1 ; c1ccc(-c2cccc(N(c3ccc(-c4ccc5sc6ccc7sc(-c8ccccc8)nc7c6c5c4)cc3)c3ccc4c(c3)sc3ccccc34)c2)cc1]

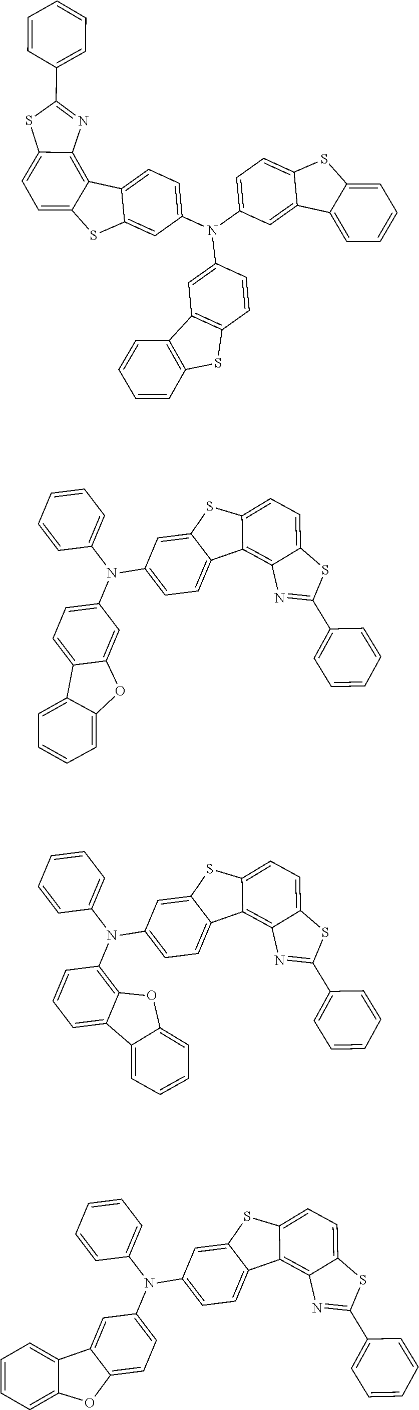 [SMILES: c1ccc(-c2nc3c(ccc4sc5cc(N(c6ccc7sc8ccccc8c7c6)c6ccc7sc8ccccc8c7c6)ccc5c43)s2)cc1 ; c1ccc(-c2nc3c(ccc4sc5cc(N(c6ccccc6)c6ccc7c(c6)oc6ccccc67)ccc5c43)s2)cc1 ; c1ccc(-c2nc3c(ccc4sc5cc(N(c6ccccc6)c6cccc7c6oc6ccccc67)ccc5c43)s2)cc1 ; c1ccc(-c2nc3c(ccc4sc5cc(N(c6ccccc6)c6ccc7oc8ccccc8c7c6)ccc5c43)s2)cc1]

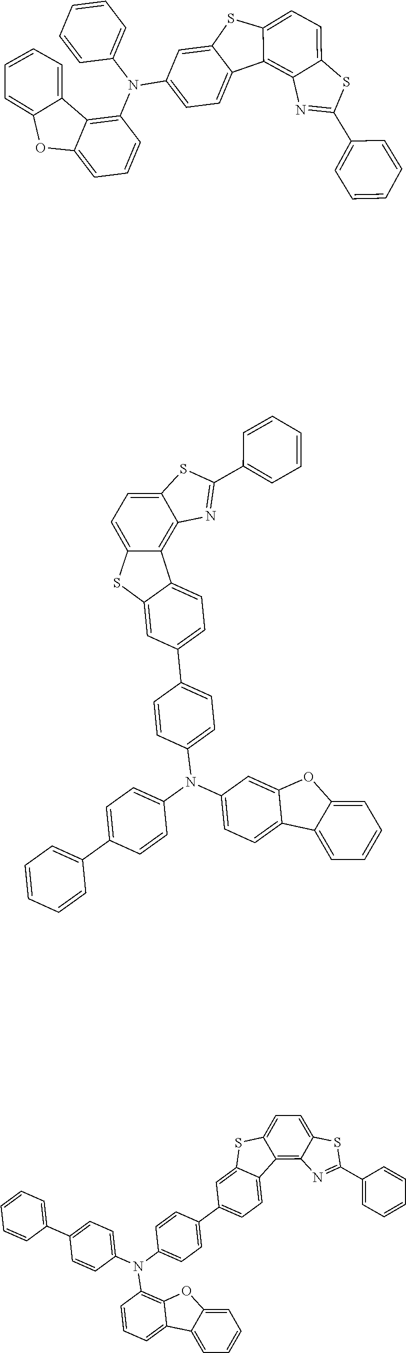 [SMILES: c1ccc(-c2nc3c(ccc4sc5cc(N(c6ccccc6)c6cccc7oc8ccccc8c67)ccc5c43)s2)cc1 ; c1ccc(-c2ccc(N(c3ccc(-c4ccc5c(c4)sc4ccc6sc(-c7ccccc7)nc6c45)cc3)c3ccc4c(c3)oc3ccccc34)cc2)cc1 ; c1ccc(-c2ccc(N(c3ccc(-c4ccc5c(c4)sc4ccc6sc(-c7ccccc7)nc6c45)cc3)c3cccc4c3oc3ccccc34)cc2)cc1]

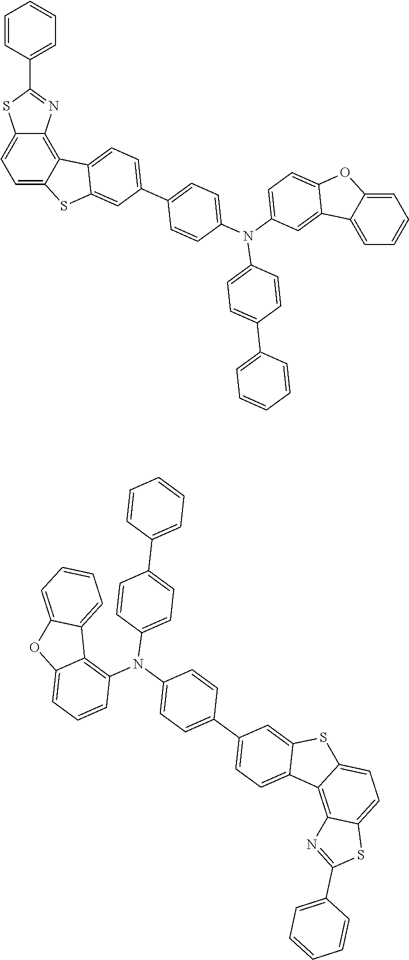 [SMILES: c1ccc(-c2ccc(N(c3ccc(-c4ccc5c(c4)sc4ccc6sc(-c7ccccc7)nc6c45)cc3)c3ccc4oc5ccccc5c4c3)cc2)cc1 ; c1ccc(-c2ccc(N(c3ccc(-c4ccc5c(c4)sc4ccc6sc(-c7ccccc7)nc6c45)cc3)c3cccc4oc5ccccc5c34)cc2)cc1]

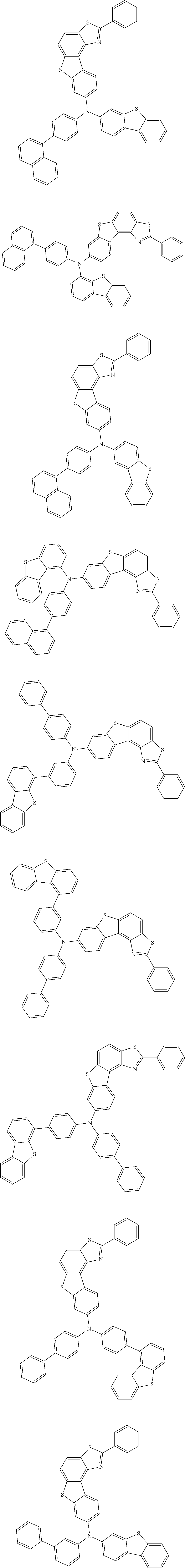

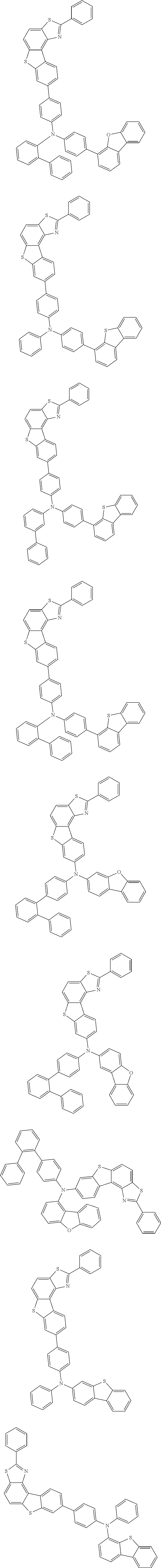

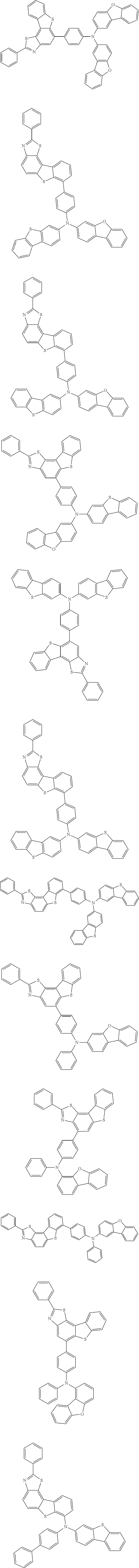

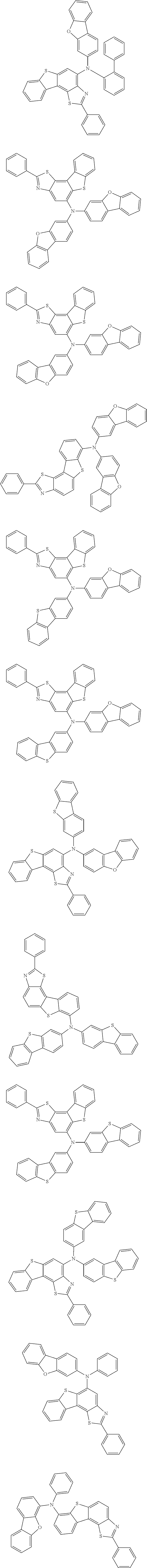

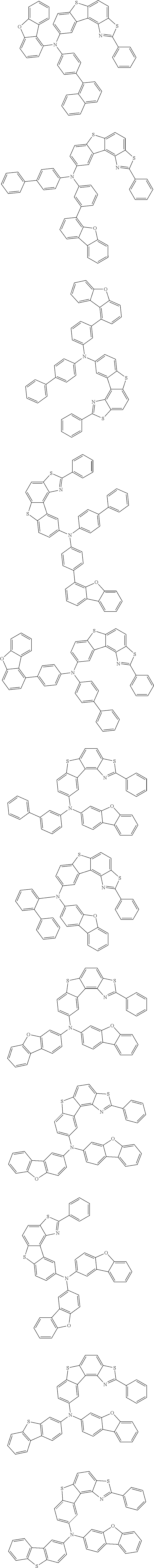

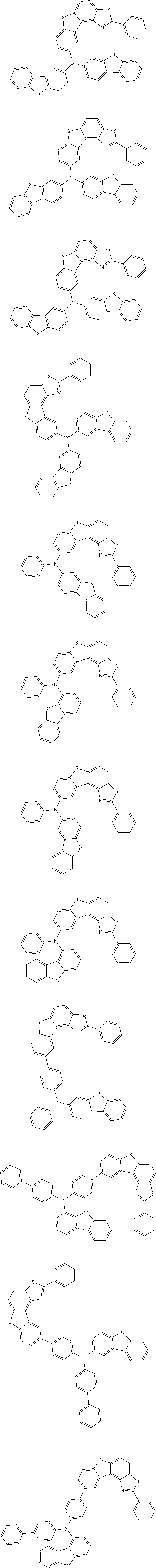

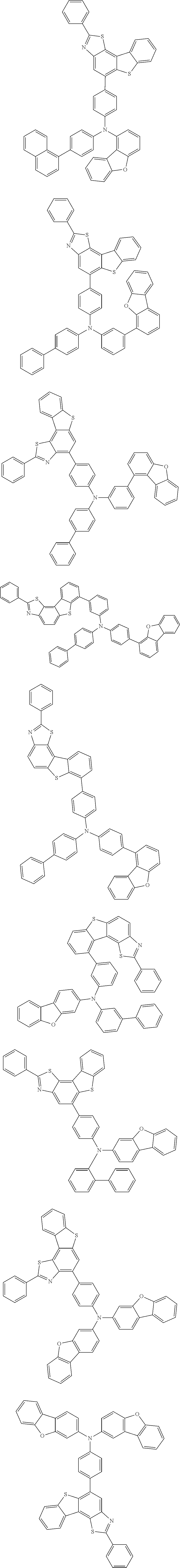

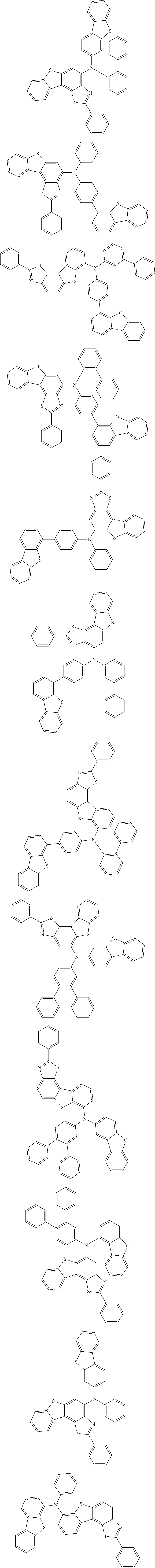

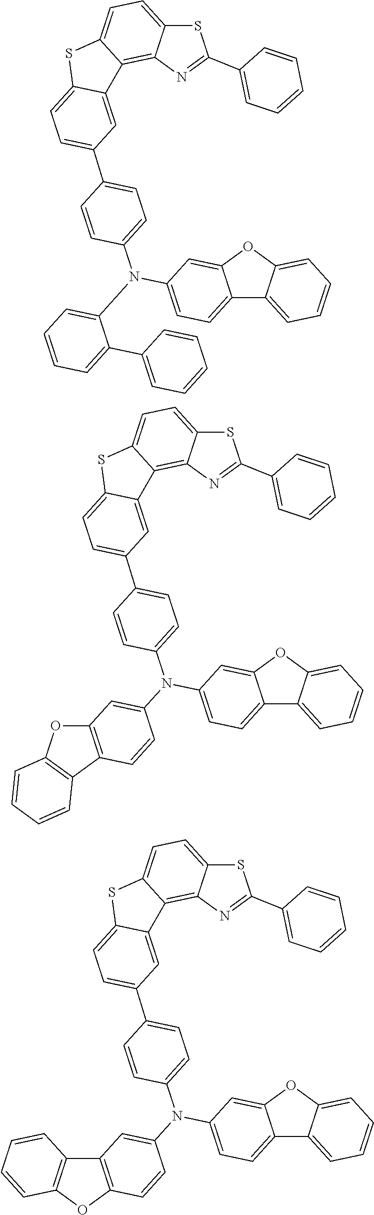

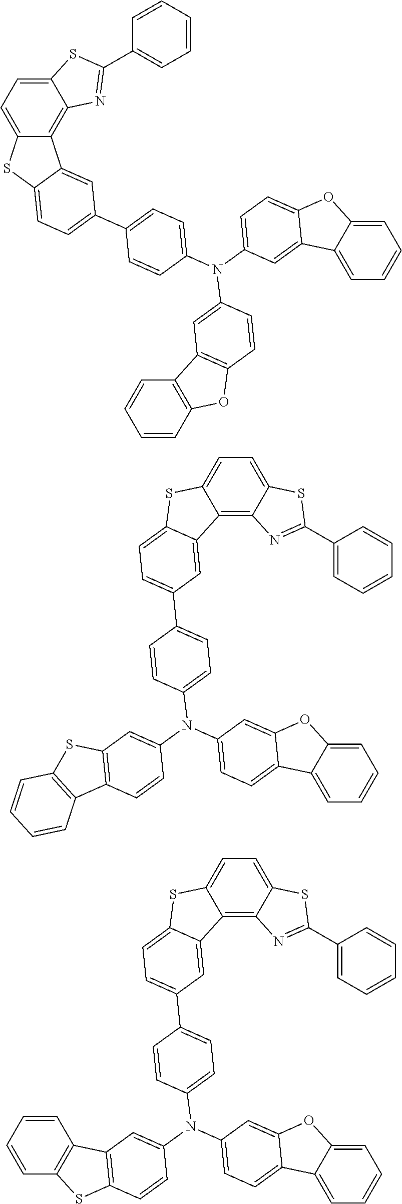 [SMILES: c1ccc(-c2nc3c(ccc4sc5ccc(-c6ccc(N(c7ccc8oc9ccccc9c8c7)c7ccc8oc9ccccc9c8c7)cc6)cc5c43)s2)cc1 ; c1ccc(-c2nc3c(ccc4sc5ccc(-c6ccc(N(c7ccc8c(c7)oc7ccccc78)c7ccc8c(c7)sc7ccccc78)cc6)cc5c43)s2)cc1 ; c1ccc(-c2nc3c(ccc4sc5ccc(-c6ccc(N(c7ccc8c(c7)oc7ccccc78)c7ccc8sc9ccccc9c8c7)cc6)cc5c43)s2)cc1]

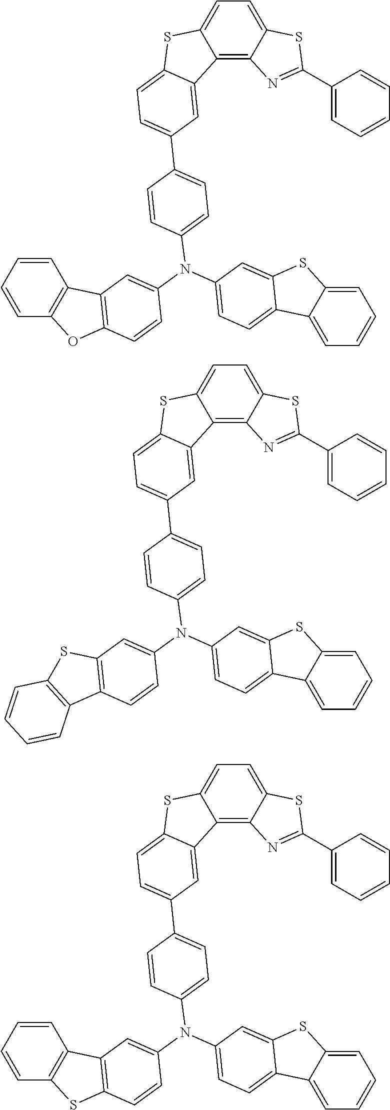

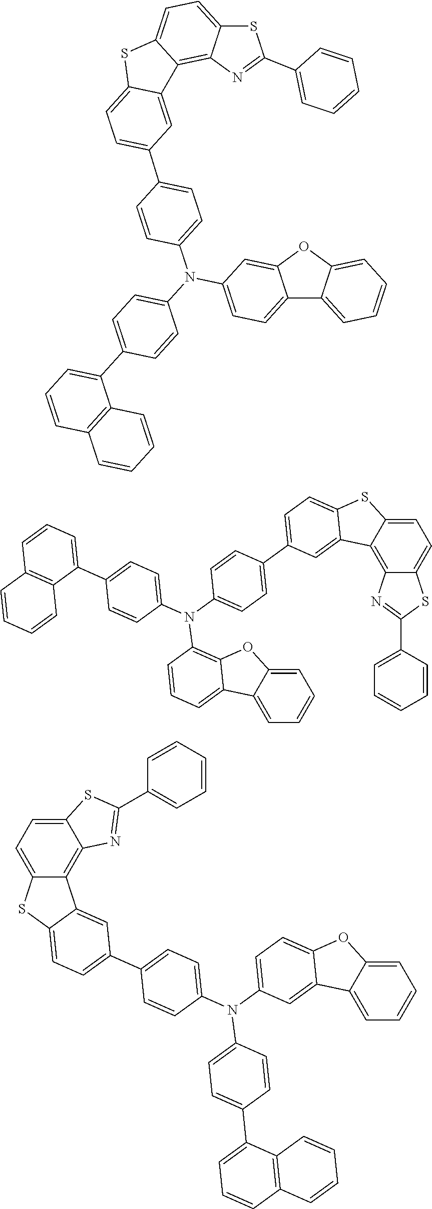

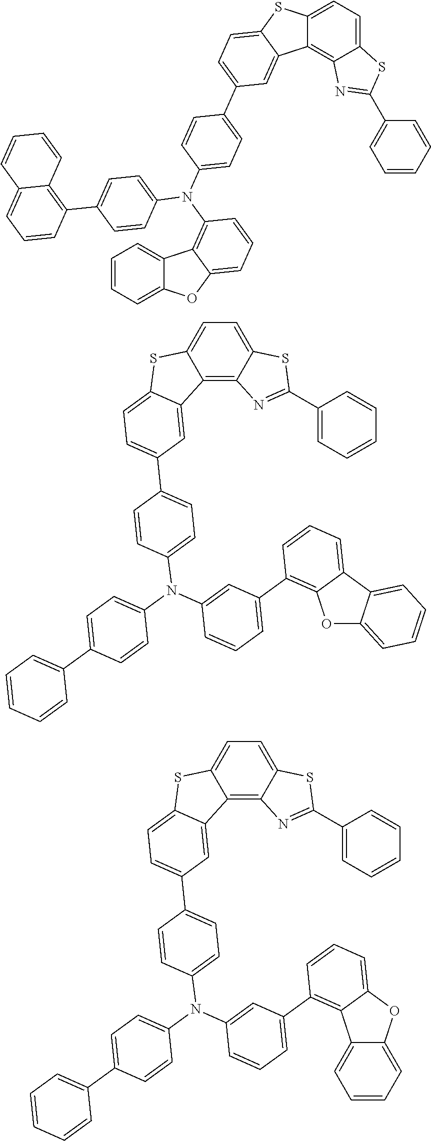

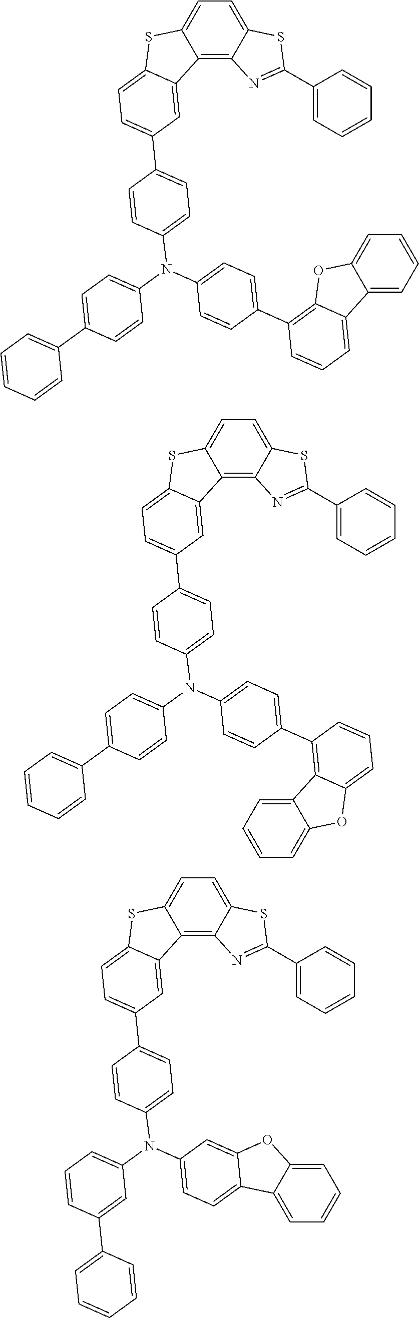 [SMILES: c1ccc(-c2ccc(N(c3ccc(-c4ccc5sc6ccc7sc(-c8ccccc8)nc7c6c5c4)cc3)c3ccc(-c4cccc5c4oc4ccccc45)cc3)cc2)cc1 ; c1ccc(-c2ccc(N(c3ccc(-c4ccc5sc6ccc7sc(-c8ccccc8)nc7c6c5c4)cc3)c3ccc(-c4cccc5oc6ccccc6c45)cc3)cc2)cc1 ; c1ccc(-c2cccc(N(c3ccc(-c4ccc5sc6ccc7sc(-c8ccccc8)nc7c6c5c4)cc3)c3ccc4c(c3)oc3ccccc34)c2)cc1]

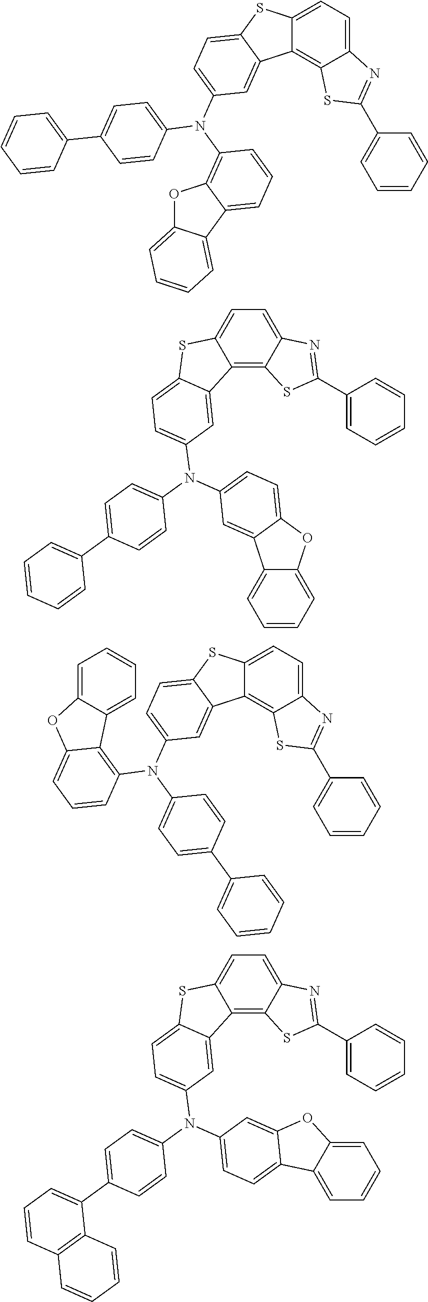

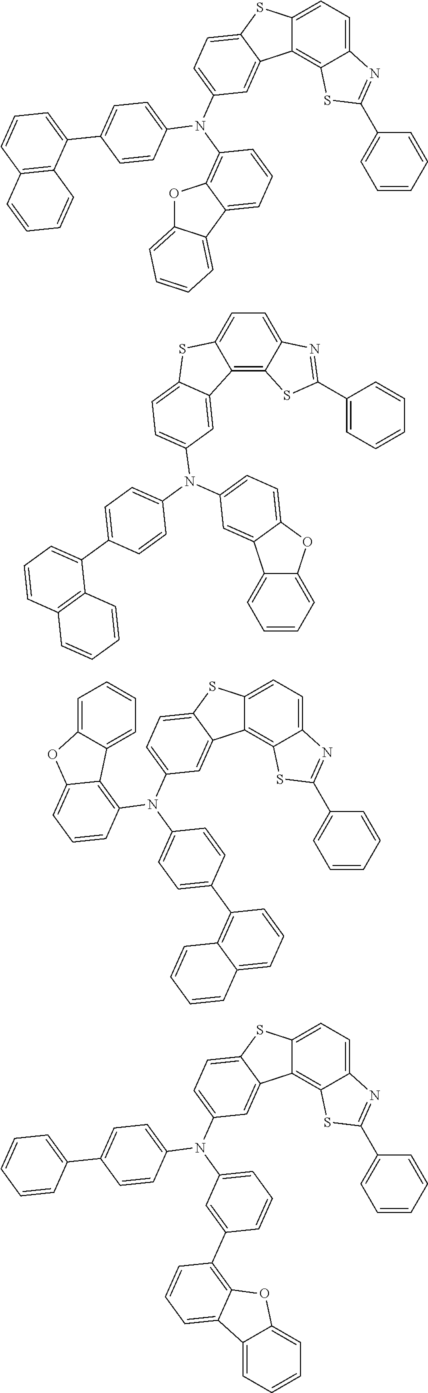

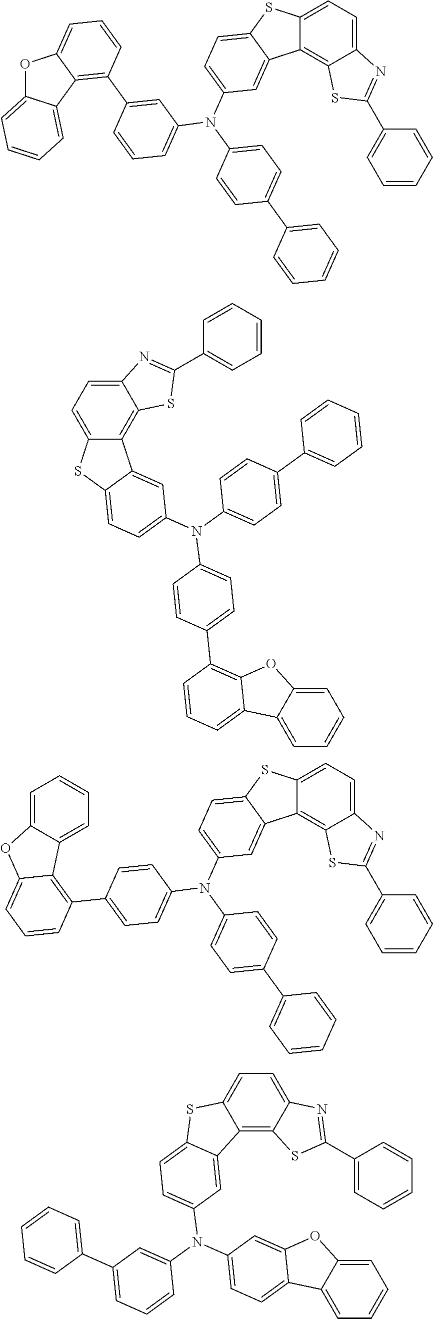

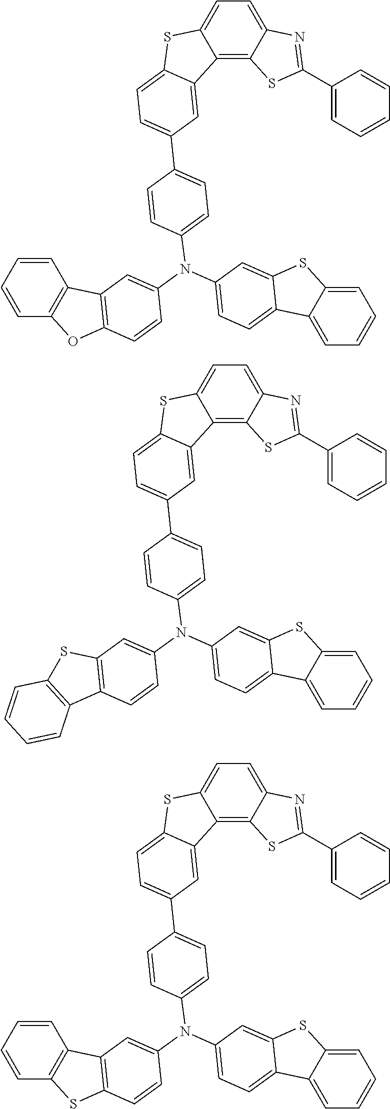

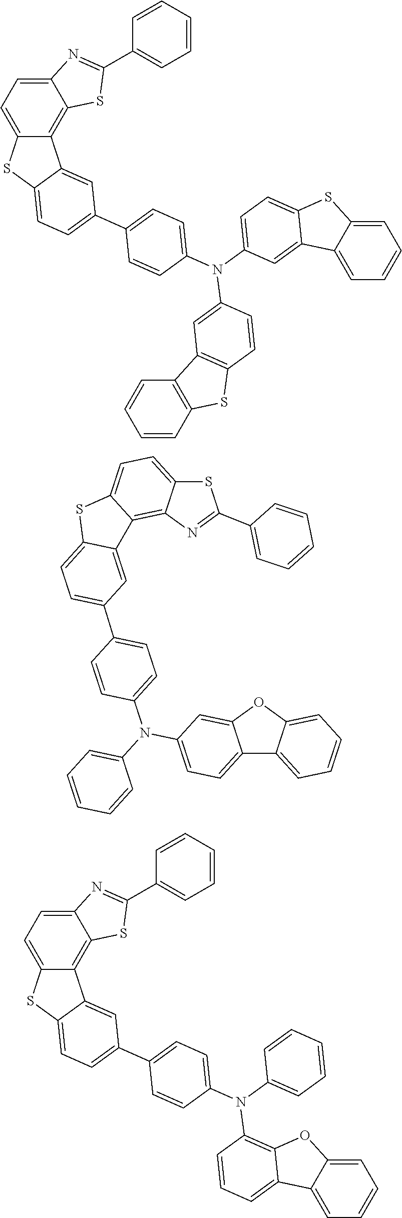

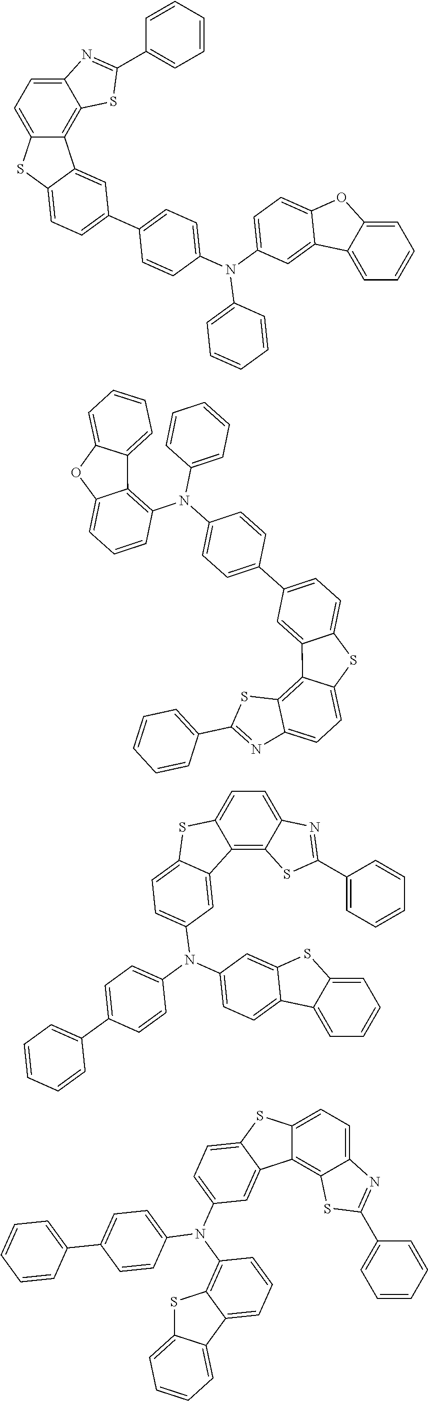

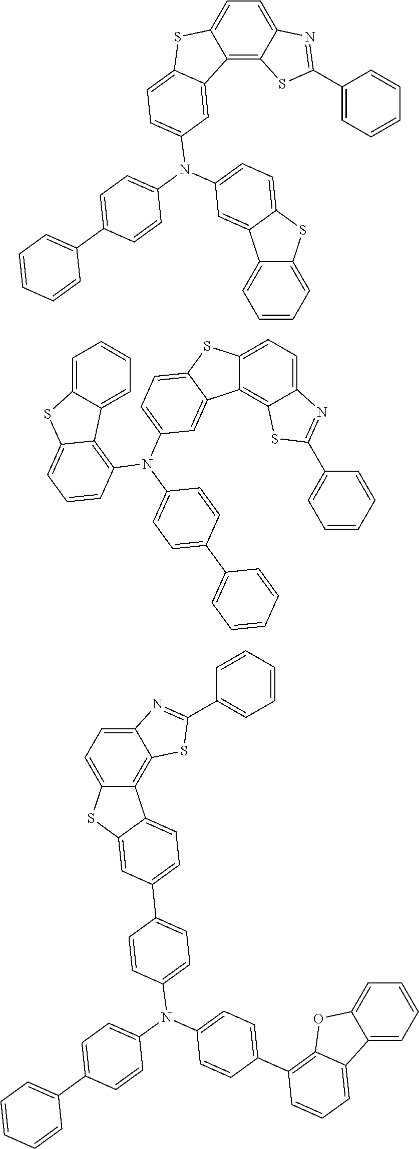

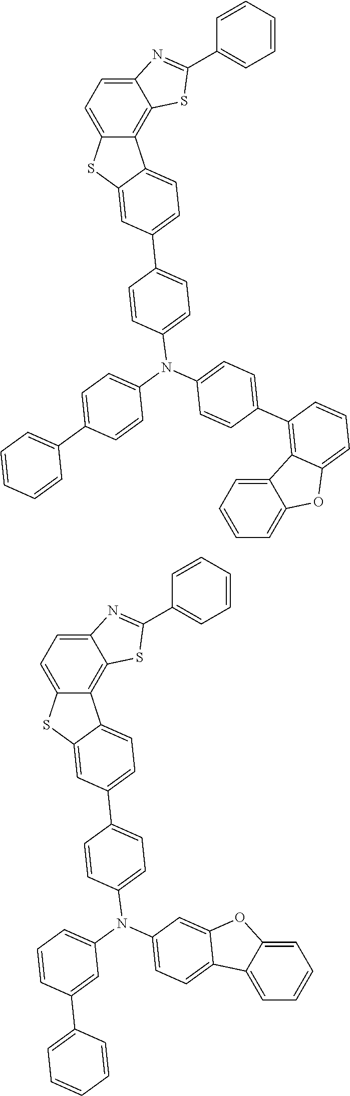

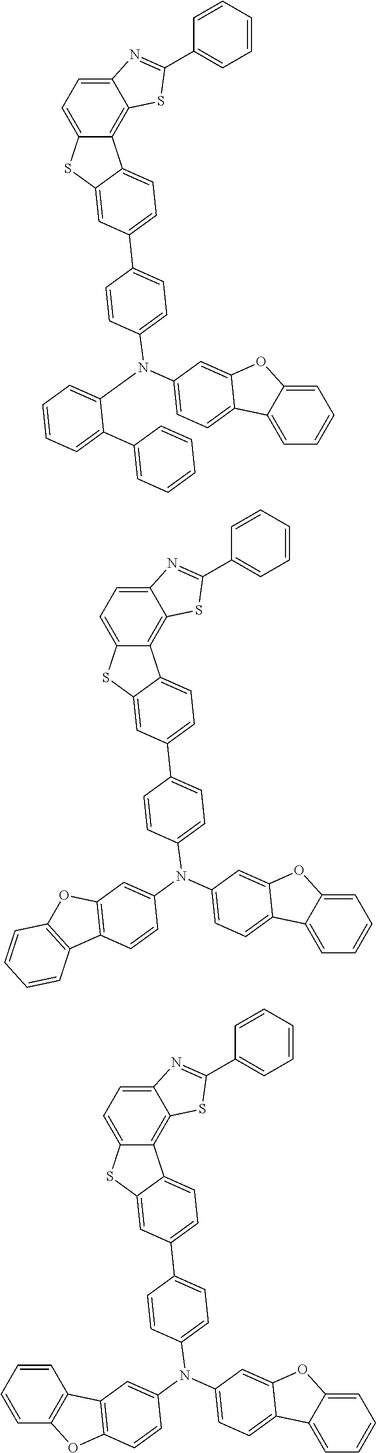 [SMILES: c1ccc(-c2nc3ccc4sc5cc(-c6ccc(N(c7ccc8c(c7)oc7ccccc78)c7ccccc7-c7ccccc7)cc6)ccc5c4c3s2)cc1 ; c1ccc(-c2nc3ccc4sc5cc(-c6ccc(N(c7ccc8c(c7)oc7ccccc78)c7ccc8c(c7)oc7ccccc78)cc6)ccc5c4c3s2)cc1 ; c1ccc(-c2nc3ccc4sc5cc(-c6ccc(N(c7ccc8c(c7)oc7ccccc78)c7ccc8oc9ccccc9c8c7)cc6)ccc5c4c3s2)cc1]

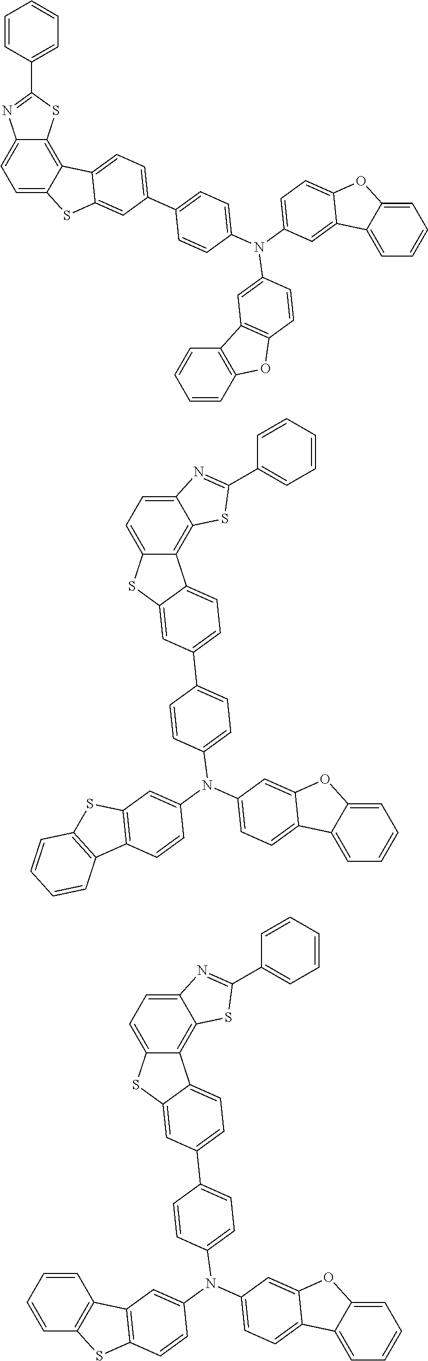 [SMILES: c1ccc(-c2nc3ccc4sc5cc(-c6ccc(N(c7ccc8oc9ccccc9c8c7)c7ccc8oc9ccccc9c8c7)cc6)ccc5c4c3s2)cc1 ; c1ccc(-c2nc3ccc4sc5cc(-c6ccc(N(c7ccc8c(c7)oc7ccccc78)c7ccc8c(c7)sc7ccccc78)cc6)ccc5c4c3s2)cc1 ; c1ccc(-c2nc3ccc4sc5cc(-c6ccc(N(c7ccc8c(c7)oc7ccccc78)c7ccc8sc9ccccc9c8c7)cc6)ccc5c4c3s2)cc1]

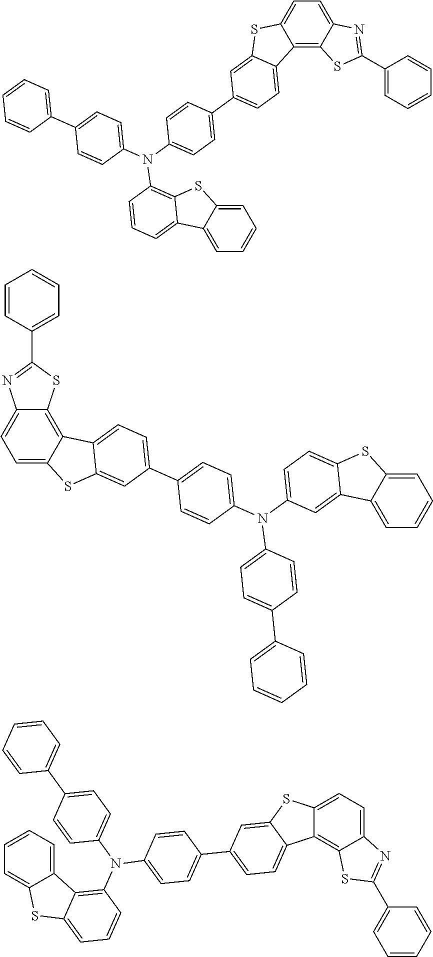 [SMILES: c1ccc(-c2ccc(N(c3ccc(-c4ccc5c(c4)sc4ccc6nc(-c7ccccc7)sc6c45)cc3)c3cccc4c3sc3ccccc34)cc2)cc1 ; c1ccc(-c2ccc(N(c3ccc(-c4ccc5c(c4)sc4ccc6nc(-c7ccccc7)sc6c45)cc3)c3ccc4sc5ccccc5c4c3)cc2)cc1 ; c1ccc(-c2ccc(N(c3ccc(-c4ccc5c(c4)sc4ccc6nc(-c7ccccc7)sc6c45)cc3)c3cccc4sc5ccccc5c34)cc2)cc1]

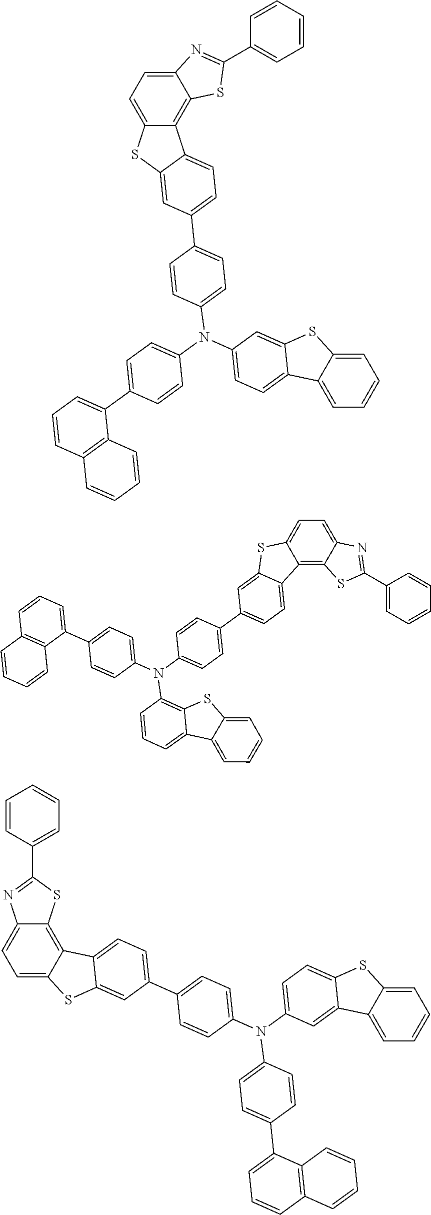 [SMILES: c1ccc(-c2nc3ccc4sc5cc(-c6ccc(N(c7ccc(-c8cccc9ccccc89)cc7)c7ccc8c(c7)sc7ccccc78)cc6)ccc5c4c3s2)cc1 ; c1ccc(-c2nc3ccc4sc5cc(-c6ccc(N(c7ccc(-c8cccc9ccccc89)cc7)c7cccc8c7sc7ccccc78)cc6)ccc5c4c3s2)cc1 ; c1ccc(-c2nc3ccc4sc5cc(-c6ccc(N(c7ccc(-c8cccc9ccccc89)cc7)c7ccc8sc9ccccc9c8c7)cc6)ccc5c4c3s2)cc1]

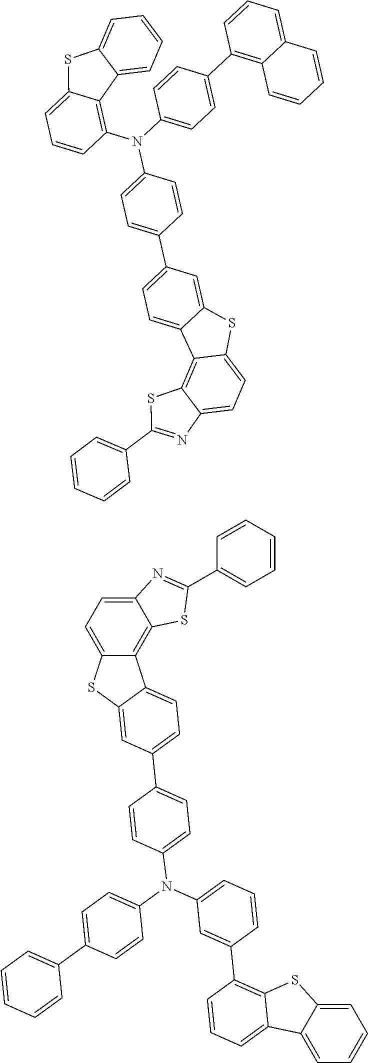 [SMILES: c1ccc(-c2nc3ccc4sc5cc(-c6ccc(N(c7ccc(-c8cccc9ccccc89)cc7)c7cccc8sc9ccccc9c78)cc6)ccc5c4c3s2)cc1 ; c1ccc(-c2ccc(N(c3ccc(-c4ccc5c(c4)sc4ccc6nc(-c7ccccc7)sc6c45)cc3)c3cccc(-c4cccc5c4sc4ccccc45)c3)cc2)cc1]

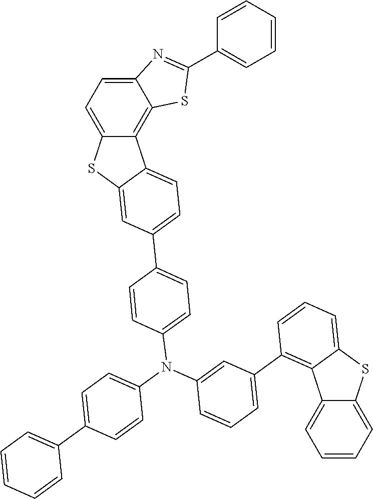 [SMILES: c1ccc(-c2ccc(N(c3ccc(-c4ccc5c(c4)sc4ccc6nc(-c7ccccc7)sc6c45)cc3)c3cccc(-c4cccc5sc6ccccc6c45)c3)cc2)cc1]

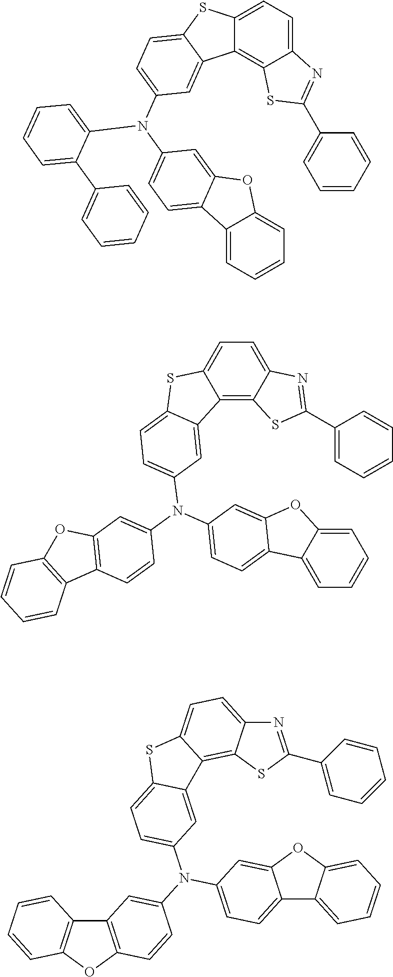 [SMILES: c1ccc(-c2nc3ccc4sc5ccc(N(c6ccc7c(c6)oc6ccccc67)c6ccccc6-c6ccccc6)cc5c4c3s2)cc1 ; c1ccc(-c2nc3ccc4sc5ccc(N(c6ccc7c(c6)oc6ccccc67)c6ccc7c(c6)oc6ccccc67)cc5c4c3s2)cc1 ; c1ccc(-c2nc3ccc4sc5ccc(N(c6ccc7c(c6)oc6ccccc67)c6ccc7oc8ccccc8c7c6)cc5c4c3s2)cc1]

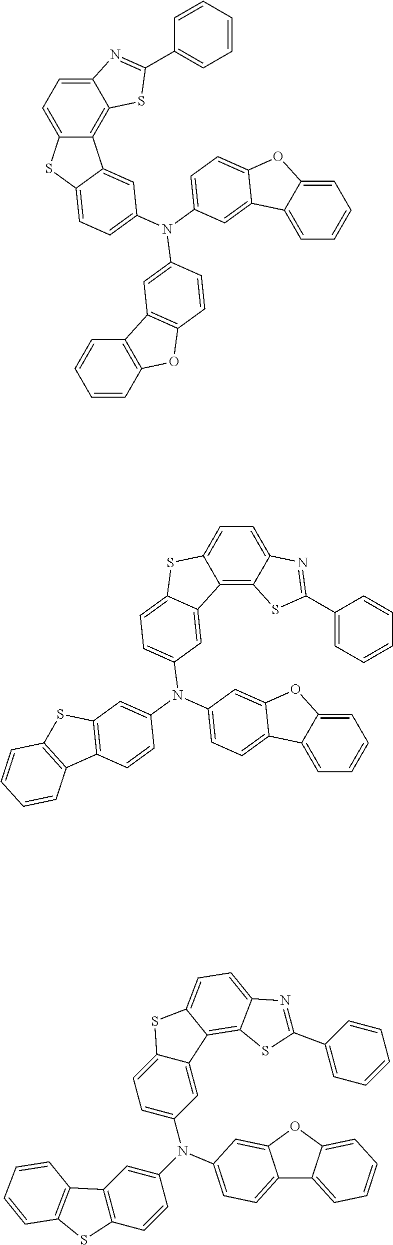 [SMILES: c1ccc(-c2nc3ccc4sc5ccc(N(c6ccc7oc8ccccc8c7c6)c6ccc7oc8ccccc8c7c6)cc5c4c3s2)cc1 ; c1ccc(-c2nc3ccc4sc5ccc(N(c6ccc7c(c6)oc6ccccc67)c6ccc7c(c6)sc6ccccc67)cc5c4c3s2)cc1 ; c1ccc(-c2nc3ccc4sc5ccc(N(c6ccc7c(c6)oc6ccccc67)c6ccc7sc8ccccc8c7c6)cc5c4c3s2)cc1]

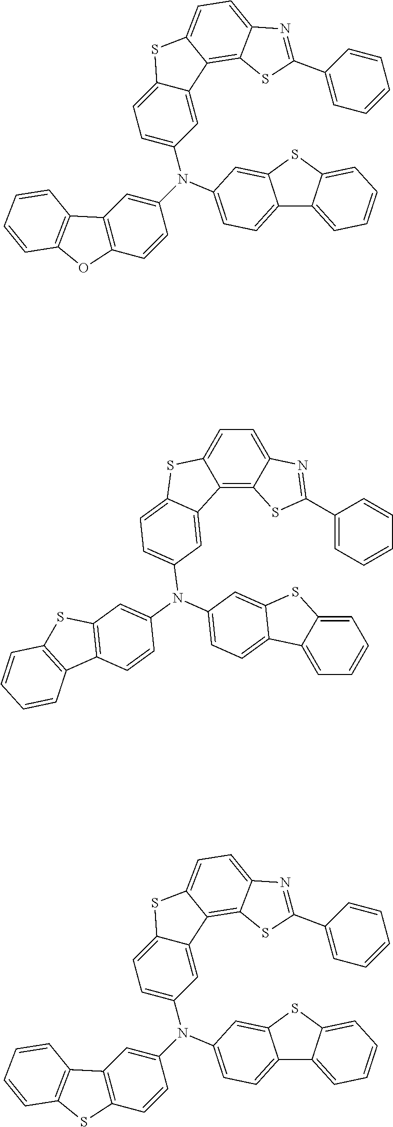 [SMILES: c1ccc(-c2nc3ccc4sc5ccc(N(c6ccc7c(c6)sc6ccccc67)c6ccc7oc8ccccc8c7c6)cc5c4c3s2)cc1 ; c1ccc(-c2nc3ccc4sc5ccc(N(c6ccc7c(c6)sc6ccccc67)c6ccc7c(c6)sc6ccccc67)cc5c4c3s2)cc1 ; c1ccc(-c2nc3ccc4sc5ccc(N(c6ccc7c(c6)sc6ccccc67)c6ccc7sc8ccccc8c7c6)cc5c4c3s2)cc1]

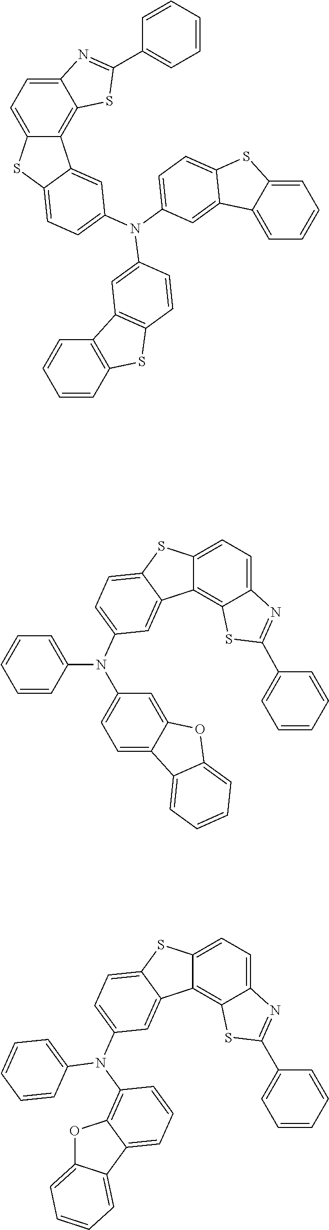 [SMILES: c1ccc(-c2nc3ccc4sc5ccc(N(c6ccc7sc8ccccc8c7c6)c6ccc7sc8ccccc8c7c6)cc5c4c3s2)cc1 ; c1ccc(-c2nc3ccc4sc5ccc(N(c6ccccc6)c6ccc7c(c6)oc6ccccc67)cc5c4c3s2)cc1 ; c1ccc(-c2nc3ccc4sc5ccc(N(c6ccccc6)c6cccc7c6oc6ccccc67)cc5c4c3s2)cc1]

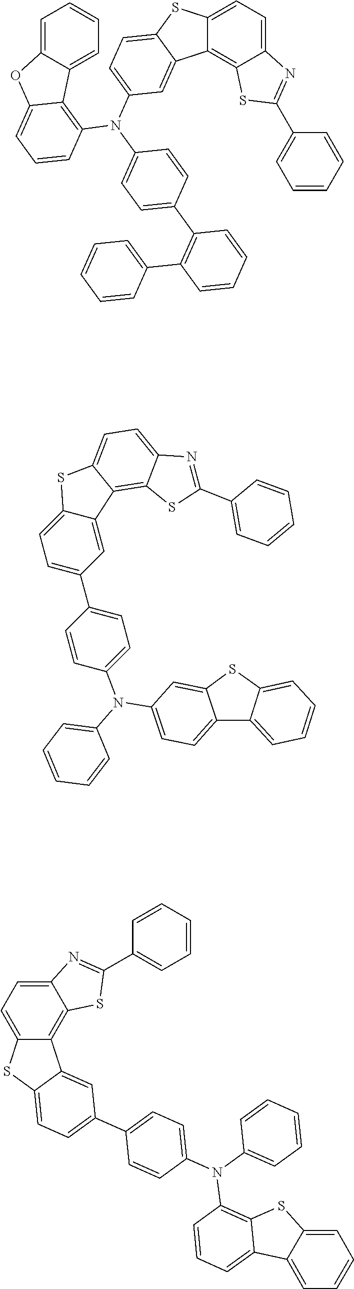 [SMILES: c1ccc(-c2nc3ccc4sc5ccc(N(c6ccc(-c7ccccc7-c7ccccc7)cc6)c6cccc7oc8ccccc8c67)cc5c4c3s2)cc1 ; c1ccc(-c2nc3ccc4sc5ccc(-c6ccc(N(c7ccccc7)c7ccc8c(c7)sc7ccccc78)cc6)cc5c4c3s2)cc1 ; c1ccc(-c2nc3ccc4sc5ccc(-c6ccc(N(c7ccccc7)c7cccc8c7sc7ccccc78)cc6)cc5c4c3s2)cc1]

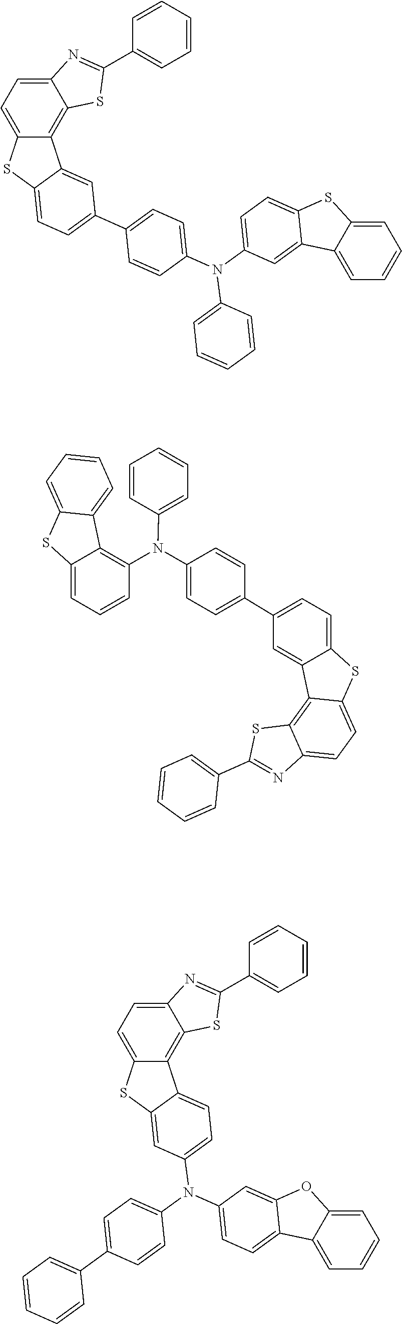 [SMILES: c1ccc(-c2nc3ccc4sc5ccc(-c6ccc(N(c7ccccc7)c7ccc8sc9ccccc9c8c7)cc6)cc5c4c3s2)cc1 ; c1ccc(-c2nc3ccc4sc5ccc(-c6ccc(N(c7ccccc7)c7cccc8sc9ccccc9c78)cc6)cc5c4c3s2)cc1 ; c1ccc(-c2ccc(N(c3ccc4c(c3)oc3ccccc34)c3ccc4c(c3)sc3ccc5nc(-c6ccccc6)sc5c34)cc2)cc1]

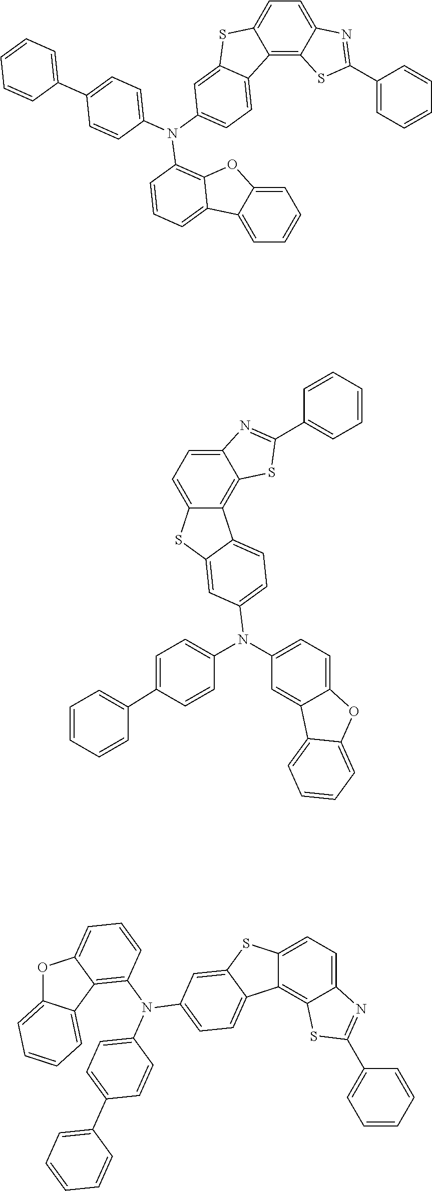 [SMILES: c1ccc(-c2ccc(N(c3ccc4c(c3)sc3ccc5nc(-c6ccccc6)sc5c34)c3cccc4c3oc3ccccc34)cc2)cc1 ; c1ccc(-c2ccc(N(c3ccc4c(c3)sc3ccc5nc(-c6ccccc6)sc5c34)c3ccc4oc5ccccc5c4c3)cc2)cc1 ; c1ccc(-c2ccc(N(c3ccc4c(c3)sc3ccc5nc(-c6ccccc6)sc5c34)c3cccc4oc5ccccc5c34)cc2)cc1]

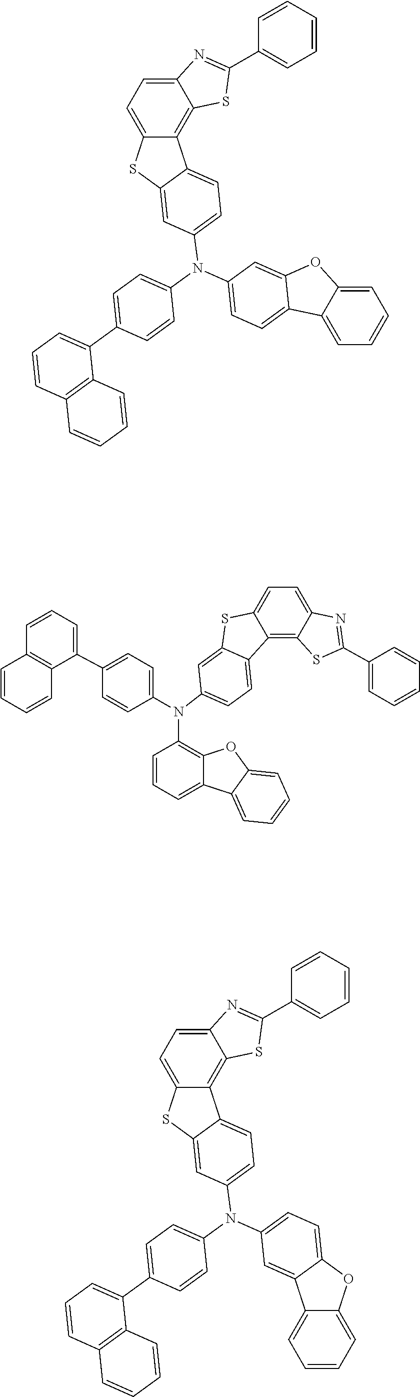 [SMILES: c1ccc(-c2nc3ccc4sc5cc(N(c6ccc(-c7cccc8ccccc78)cc6)c6ccc7c(c6)oc6ccccc67)ccc5c4c3s2)cc1 ; c1ccc(-c2nc3ccc4sc5cc(N(c6ccc(-c7cccc8ccccc78)cc6)c6cccc7c6oc6ccccc67)ccc5c4c3s2)cc1 ; c1ccc(-c2nc3ccc4sc5cc(N(c6ccc(-c7cccc8ccccc78)cc6)c6ccc7oc8ccccc8c7c6)ccc5c4c3s2)cc1]

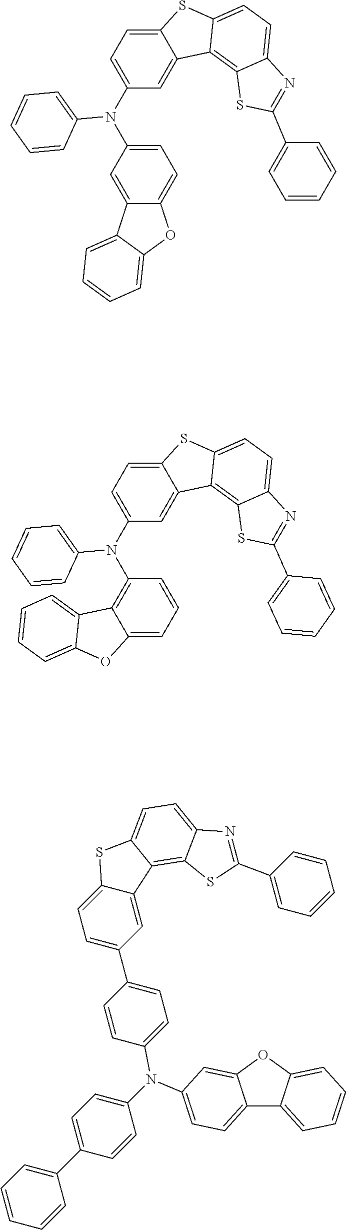 [SMILES: c1ccc(-c2nc3ccc4sc5ccc(N(c6ccccc6)c6ccc7oc8ccccc8c7c6)cc5c4c3s2)cc1 ; c1ccc(-c2nc3ccc4sc5ccc(N(c6ccccc6)c6cccc7oc8ccccc8c67)cc5c4c3s2)cc1 ; c1ccc(-c2ccc(N(c3ccc(-c4ccc5sc6ccc7nc(-c8ccccc8)sc7c6c5c4)cc3)c3ccc4c(c3)oc3ccccc34)cc2)cc1]

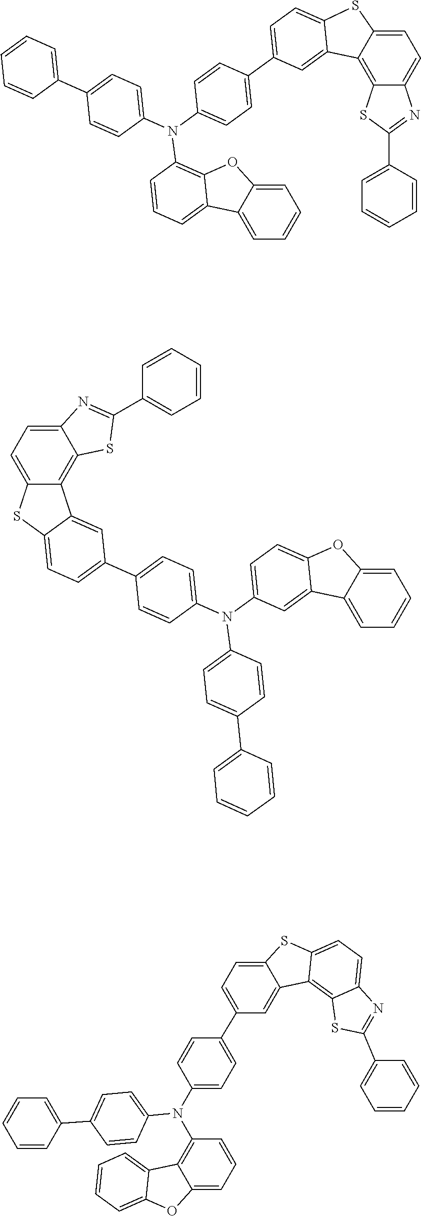 [SMILES: c1ccc(-c2ccc(N(c3ccc(-c4ccc5sc6ccc7nc(-c8ccccc8)sc7c6c5c4)cc3)c3cccc4c3oc3ccccc34)cc2)cc1 ; c1ccc(-c2ccc(N(c3ccc(-c4ccc5sc6ccc7nc(-c8ccccc8)sc7c6c5c4)cc3)c3ccc4oc5ccccc5c4c3)cc2)cc1 ; c1ccc(-c2ccc(N(c3ccc(-c4ccc5sc6ccc7nc(-c8ccccc8)sc7c6c5c4)cc3)c3cccc4oc5ccccc5c34)cc2)cc1]

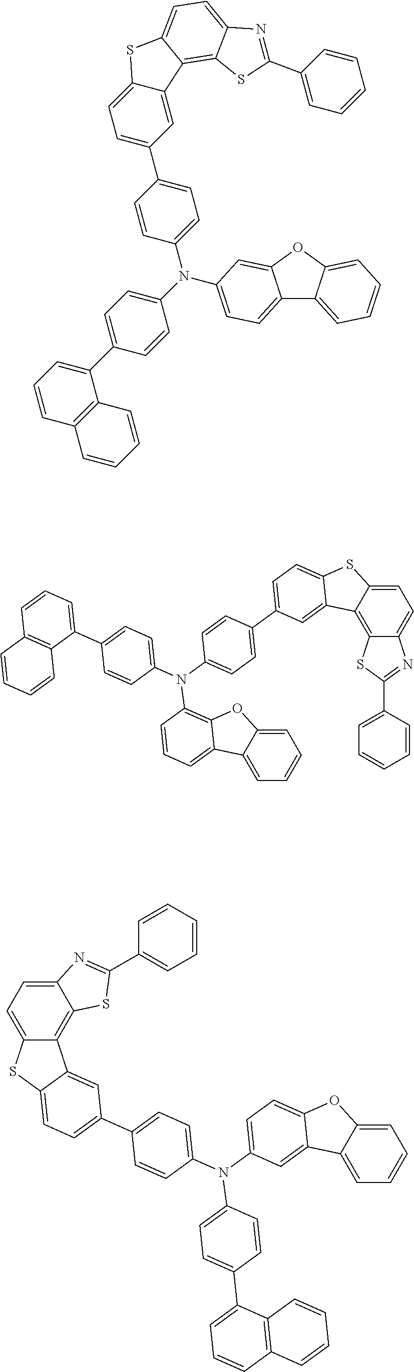 [SMILES: c1ccc(-c2nc3ccc4sc5ccc(-c6ccc(N(c7ccc(-c8cccc9ccccc89)cc7)c7ccc8c(c7)oc7ccccc78)cc6)cc5c4c3s2)cc1 ; c1ccc(-c2nc3ccc4sc5ccc(-c6ccc(N(c7ccc(-c8cccc9ccccc89)cc7)c7cccc8c7oc7ccccc78)cc6)cc5c4c3s2)cc1 ; c1ccc(-c2nc3ccc4sc5ccc(-c6ccc(N(c7ccc(-c8cccc9ccccc89)cc7)c7ccc8oc9ccccc9c8c7)cc6)cc5c4c3s2)cc1]

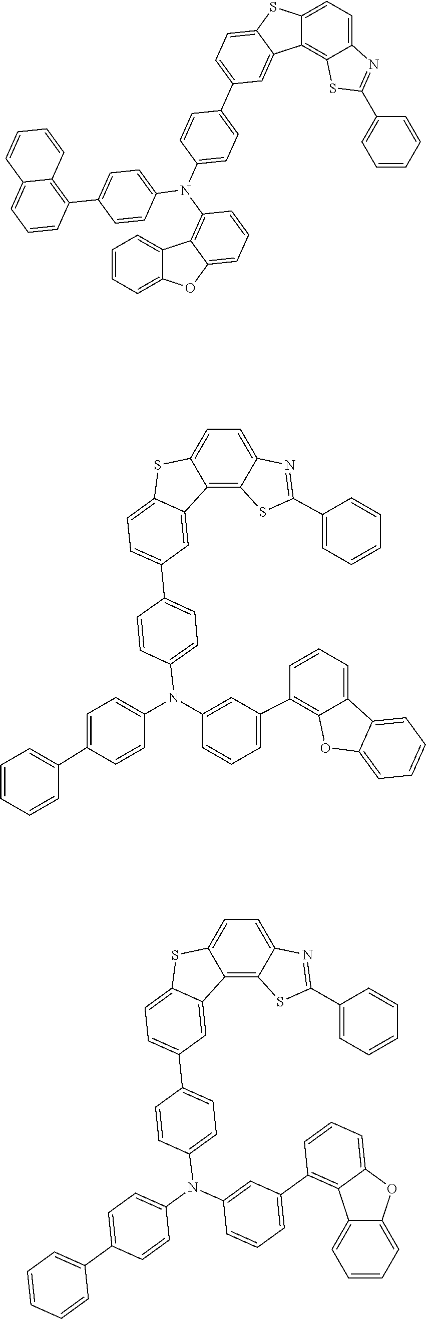 [SMILES: c1ccc(-c2nc3ccc4sc5ccc(-c6ccc(N(c7ccc(-c8cccc9ccccc89)cc7)c7cccc8oc9ccccc9c78)cc6)cc5c4c3s2)cc1 ; c1ccc(-c2ccc(N(c3ccc(-c4ccc5sc6ccc7nc(-c8ccccc8)sc7c6c5c4)cc3)c3cccc(-c4cccc5c4oc4ccccc45)c3)cc2)cc1 ; c1ccc(-c2ccc(N(c3ccc(-c4ccc5sc6ccc7nc(-c8ccccc8)sc7c6c5c4)cc3)c3cccc(-c4cccc5oc6ccccc6c45)c3)cc2)cc1]

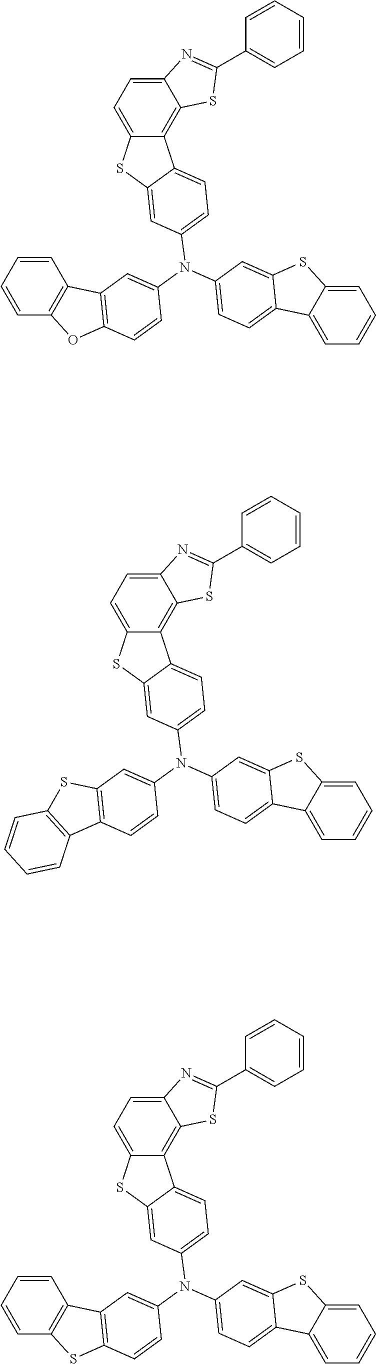 [SMILES: c1ccc(-c2nc3ccc4sc5cc(N(c6ccc7c(c6)sc6ccccc67)c6ccc7oc8ccccc8c7c6)ccc5c4c3s2)cc1 ; c1ccc(-c2nc3ccc4sc5cc(N(c6ccc7c(c6)sc6ccccc67)c6ccc7c(c6)sc6ccccc67)ccc5c4c3s2)cc1 ; c1ccc(-c2nc3ccc4sc5cc(N(c6ccc7c(c6)sc6ccccc67)c6ccc7sc8ccccc8c7c6)ccc5c4c3s2)cc1]

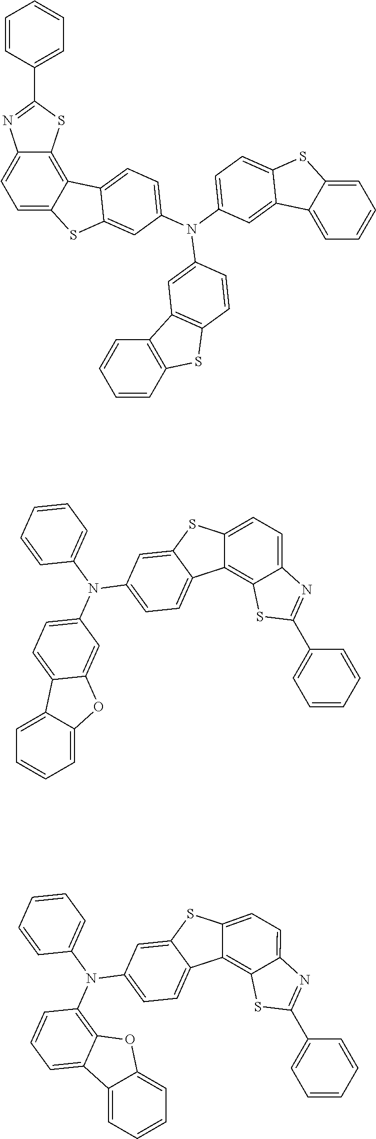 [SMILES: c1ccc(-c2nc3ccc4sc5cc(N(c6ccc7sc8ccccc8c7c6)c6ccc7sc8ccccc8c7c6)ccc5c4c3s2)cc1 ; c1ccc(-c2nc3ccc4sc5cc(N(c6ccccc6)c6ccc7c(c6)oc6ccccc67)ccc5c4c3s2)cc1 ; c1ccc(-c2nc3ccc4sc5cc(N(c6ccccc6)c6cccc7c6oc6ccccc67)ccc5c4c3s2)cc1]

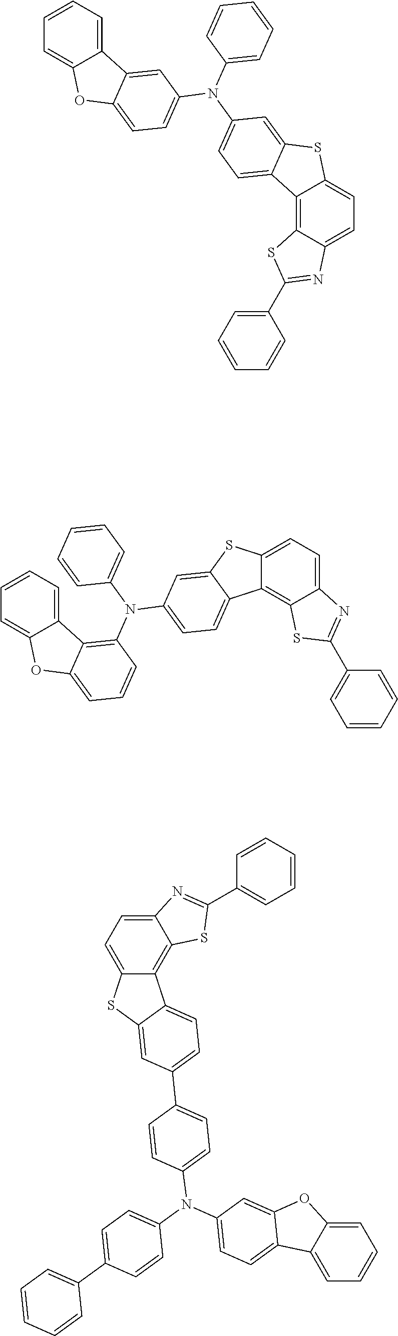 [SMILES: c1ccc(-c2nc3ccc4sc5cc(N(c6ccccc6)c6ccc7oc8ccccc8c7c6)ccc5c4c3s2)cc1 ; c1ccc(-c2nc3ccc4sc5cc(N(c6ccccc6)c6cccc7oc8ccccc8c67)ccc5c4c3s2)cc1 ; c1ccc(-c2ccc(N(c3ccc(-c4ccc5c(c4)sc4ccc6nc(-c7ccccc7)sc6c45)cc3)c3ccc4c(c3)oc3ccccc34)cc2)cc1]

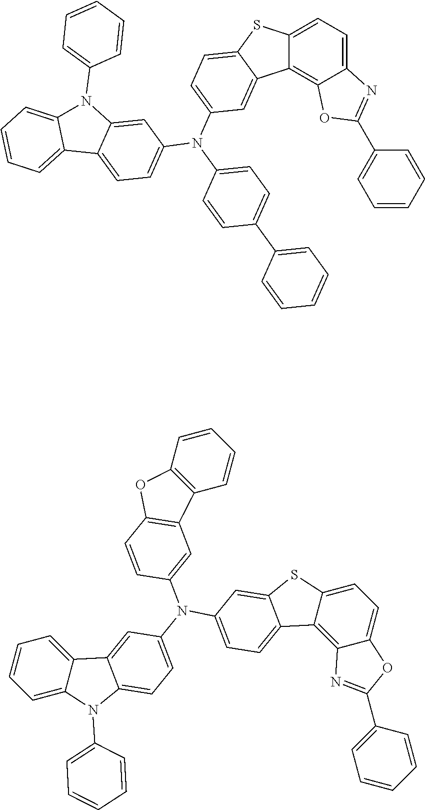 [SMILES: c1ccc(-c2ccc(N(c3ccc4sc5ccc6nc(-c7ccccc7)oc6c5c4c3)c3ccc4c5ccccc5n(-c5ccccc5)c4c3)cc2)cc1 ; c1ccc(-c2nc3c(ccc4sc5cc(N(c6ccc7oc8ccccc8c7c6)c6ccc7c(c6)c6ccccc6n7-c6ccccc6)ccc5c43)o2)cc1]

In addition, provided is a method for preparing a compound of Chemical Formula 1.

For example, Chemical Formula 1 can be prepared by a preparation method as shown in Reaction Scheme 1 below.

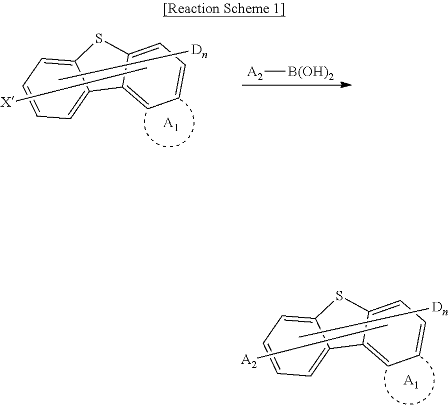

In the above, the definitions of other substituents except for X′ are the same as defined in the Chemical Formula 1, and X′ is halogen, preferably chloro or bromo.

The Reaction Scheme 1 is a Suzuki coupling reaction, and preferably performed in the presence of a palladium catalyst and a base. In addition, the reactive group for the Suzuki coupling reaction can be appropriately changed as known in the art.

Alternatively, when A₂ is Chemical Formula 1-c and L₂ is a single bond in Chemical Formula 1, the compound of Chemical Formula 1 can be prepared by a preparation method as shown in Reaction Scheme 2 below.

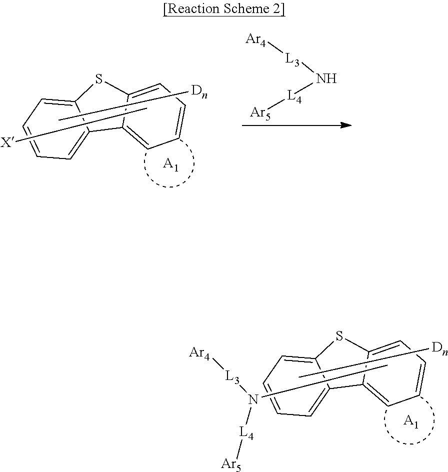

In the above, the definitions of other substituents except for X′ are the same as defined in the Chemical Formula 1, and X′ is halogen, preferably chloro or bromo.

The Reaction Scheme 2 is an amine substitution reaction, and preferably performed in the presence of a palladium catalyst and a base. In addition, the reactive group for the amine substitution reaction can be appropriately changed as known in the art.

The preparation method can be more specifically described in Preparation Examples described below.

In addition, provided is an organic light emitting device including the compound of Chemical Formula 1. As an example, provided is an organic light emitting device including: a first electrode; a second electrode that is opposite to the first electrode; and one or more organic material layers that are between the first electrode and the second electrode, wherein at least one layer of the one or more organic material layers includes the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure including a hole injection layer, a hole transport layer, a light emitting layer, an electron transport layer, an electron injection layer, and the like as the organic material layer. However, the structure of the organic light emitting device is not limited thereto, and it can include a smaller number of organic layers.

In addition, the organic material layer can include a light emitting layer, and the light emitting layer includes the compound of Chemical Formula 1. In particular, the compound according to the present disclosure can be used as a host for the light emitting layer.

In addition, the organic material layer can include a hole injection layer, a hole transport layer, or an electron blocking layer, and the hole injection layer, the hole transport layer, or the electron blocking layer includes the compound of Chemical Formula 1.

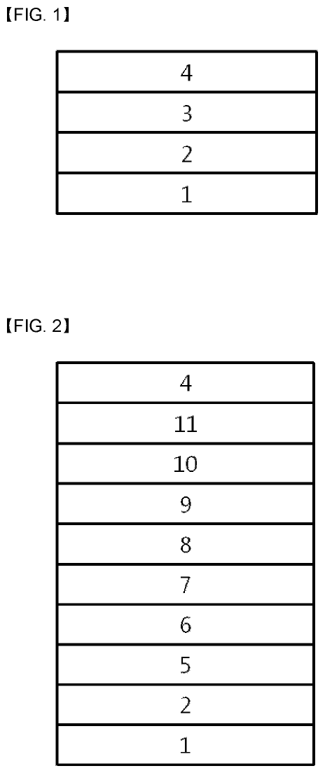

Further, the organic light emitting device according to the present disclosure can be a normal type organic light emitting device in which an anode, one or more organic material layers and a cathode are sequentially stacked on a substrate. Further, the organic light emitting device according to the present disclosure can be an inverted type organic light emitting device in which a cathode, one or more organic material layers and an anode are sequentially stacked on a substrate. For example, the structure of an organic light emitting device according to an embodiment of the present disclosure is illustrated in FIGS. 1 to 3.

FIG. 1 shows an example of an organic light emitting device including a substrate 1, an anode 2, an organic material layer 3, and a cathode 4. In such a structure, the compound of Chemical Formula 1 can be included in the light emitting layer.

FIG. 2 shows an example of an organic light emitting device including a substrate 1, an anode 2, a hole injection layer 5, a hole transport layer 6, an electron blocking layer 7, a light emitting layer 8, a hole blocking layer 9, an electron transport layer 10, an electron injection layer 11, and a cathode 4. In such a structure, the compound of Chemical Formula 1 can be included in at least one layer of the hole injection layer, the hole transport layer, the electron blocking layer, the light emitting layer, the hole blocking layer, the electron transport layer and the electron injection layer.

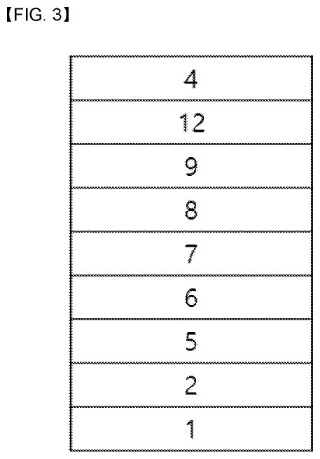

FIG. 3 shows an example of an organic light emitting device including a substrate 1, an anode 2, a hole injection layer 5, a hole transport layer 6, an electron blocking layer 7, a light emitting layer 8, a hole blocking layer 9, an electron injection and transport layer 12, and a cathode 4. In such a structure, the compound of Chemical Formula 1 can be included in at least one layer of the hole injection layer, the hole transport layer, the electron blocking layer, the light emitting layer, the hole blocking layer, and the electron injection and transport layer. For example, it can be included in the light emitting layer or the electron blocking layer.

The organic light emitting device according to the present disclosure can be manufactured using materials and methods known in the art, except that at least one layer of the organic material layers includes the compound of Chemical Formula 1. Moreover, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic material layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

For example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO2:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole-injecting effect to the light emitting layer or the light emitting material, prevents excitons produced in the light emitting layer from moving to an electron injection layer or the electron injection material, and is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

In addition, the hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The electron blocking layer serves to improve the efficiency of an organic light emitting device by suppressing electron injected from the cathode from being transferred to the anode without recombination in the light emitting layer. A material having the electron affinity lower than that of the electron transport layer is preferable for the electron blocking layer. Preferably, the material of Chemical Formula 1 of the present disclosure can be used as the electron blocking material.

The light emitting material is suitably a material capable of emitting light in a visible ray region by receiving holes and electrons from the hole transport layer and the electron transport layer, respectively, to combine them, and having good quantum efficiency to fluorescence or phosphorescence. Specific examples thereof include 8-hydroxy-quinoline aluminum complex (Alq₃); a carbazole-based compound; a dimerized styryl compound; BAlq; a 10-hydroxybenzo quinoline-metal compound; a benzoxazole-, benzothiazole- and benzimidazole-based compound; a poly(p-phenylenevinylene) (PPV)-based polymer; a spiro compound; polyfluorene, rubrene, and the like, but are not limited thereto.

In addition, the light emitting layer can include a host material and a dopant material. The host material can be a fused aromatic ring derivative or a heterocycle-containing compound. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto. In particular, the compound of Chemical Formula 1 can be used as a host material for the light emitting layer in the present disclosure, and in this case, low voltage, high efficiency, and/or high lifespan of the organic light emitting device can be achieved.

Specifically, in Chemical Formula 1, when A₂ is a triazine substituent of Chemical Formula 1-b, it can be suitable for use as an N-type host material, and when A₂ is an amine substituent of Chemical Formula 1-c, it can be suitable for use as a P-type host material. Accordingly, in Chemical Formula 1, at least one compound in which A₂ is a triazine substituent of Chemical Formula 1-b and at least one compound in which A₂ is an amine substituent of Chemical Formula 1-c can be simultaneously included in the light emitting layer.

The dopant material includes an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer which receives electrons from an electron injection layer and transports the electrons to a light emitting layer, and an electron transport material used is suitably a material which can receive electrons well from a cathode and transfer the electrons to a light emitting layer and has large mobility for electrons. Specifically, examples thereof can include an Al complex of 8-hydroxyquinoline; a complex including Alq₃; an organic radical compound; a hydroxyflavone-metal complex, and the like, but are not limited thereto. The electron transport layer can be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

According to one embodiment of the present disclosure, the electron transport material and the electron injection material can be simultaneously deposited to form an electron injection and transport layer as a single layer.

The organic light emitting device according to the present disclosure can be a bottom emission device, a top emission device, or a double-sided emission device, and in particular, can be a bottom emission device requiring relatively high luminous efficiency.

In addition, the compound of Chemical Formula 1 can be included in an organic solar cell or an organic transistor in addition to an organic light emitting device.

The preparation of the compound of Chemical Formula 1 and the organic light emitting device including the same will be described in detail in the following examples. However, these examples are presented for illustrative purposes only, and are not intended to limit the scope of the present disclosure.

### EXAMPLES

### Preparation Examples: Preparation of Core of Compound of Chemical Formula 1

### Synthesis Scheme of Preparation Examples 1 to 4

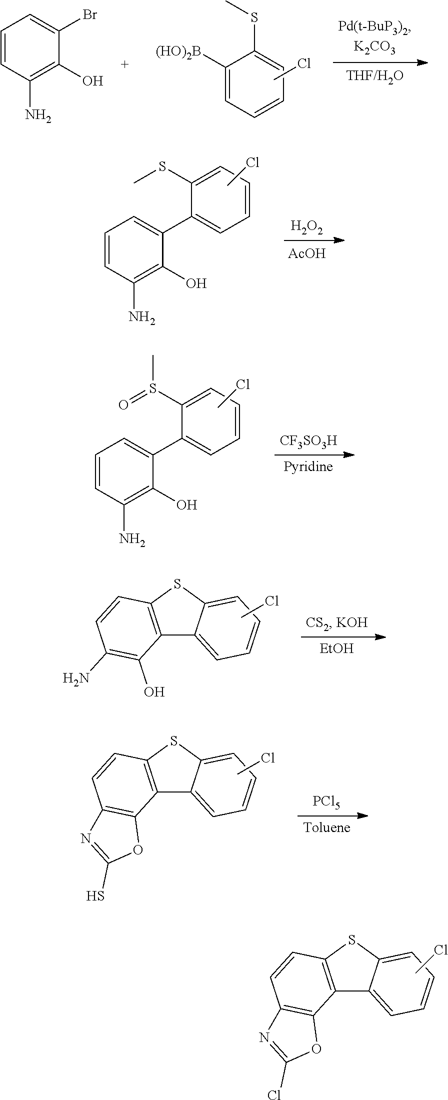

### Preparation Example 1: Synthesis of Chemical Formula AA

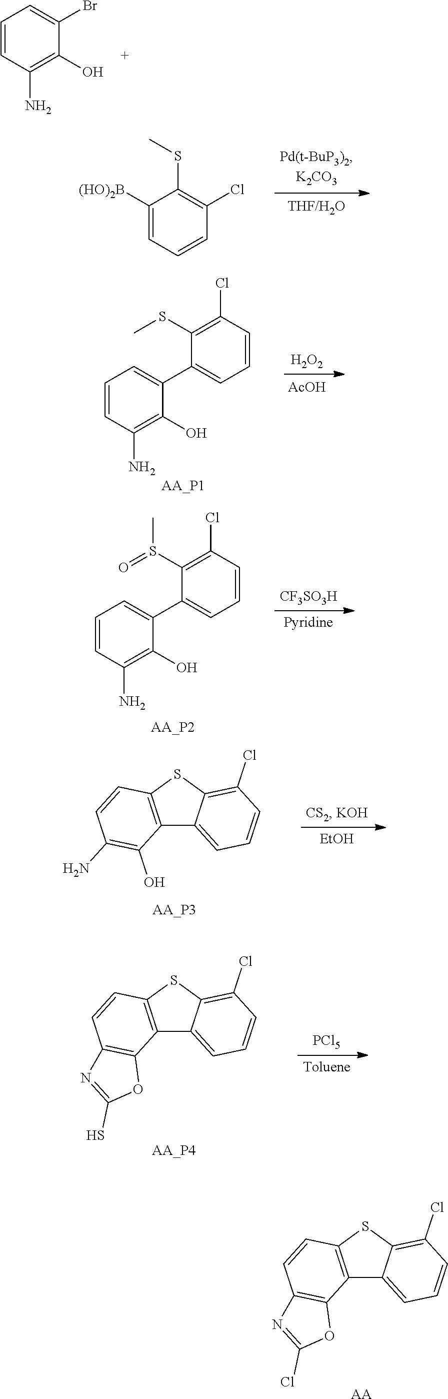

2-amino-6-bromophenol (15 g, 79.8 mmol) and (3-chloro-2-(methylthio)-phenyl)boronic acid (17 g, 83.8 mmol) were added to 300 ml of THF under a nitrogen atmosphere, and the mixture was stirred and refluxed. Then, potassium carbonate (33.1 g, 239.3 mmol) was dissolved in 99 ml of water, and then added thereto. Thereafter, it was stirred sufficiently, followed by adding bis(tri-tert-butylphosphine)palladium(0) (0.4 g, 0.8 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16.1 g of Chemical Formula AA_P1 (yield 76%, MS: [M+H]+=266).

Chemical Formula AA_P1 (15 g, 56.6 mmol) and hydrogen peroxide (3.8 g, 113.2 mmol) were added to 300 ml of acetic acid under a nitrogen atmosphere, and the mixture was stirred and refluxed. After 10 hours of reaction, cooling was performed to room temperature, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.8 g of Chemical Formula AA_P2 (yield 74%, MS: [M+H]+=282).

Chemical Formula AA_P2 (15 g, 53.2 mmol) and trifluoromethanesulfonic acid (12 g, 79.9 mmol) were added to 300 ml of pyridine under a nitrogen atmosphere, and stirred at room temperature. After 11 hours of reaction, it was poured into 600 ml of water for solidification, and then filtered. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 8.2 g of Chemical Formula AA_P3 (yield 62%, MS: [M+H]+=250).

Chemical Formula AA_P3 (15 g, 60.2 mmol), carbon disulfide (5.5 g, 72 mmol), and potassium hydroxide (4.1 g, 77 mmol) were added to 150 ml of EtOH under a nitrogen atmosphere, and the mixture was stirred and refluxed. After 12 hours of reaction, cooling was performed to room temperature, and then the organic solvent was distilled under reduced pressure. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.9 g of Chemical Formula AA_P4 (yield 64%, MS: [M+H]+=258).

Chemical Formula AA_P4 (15 g, 58.4 mmol) and phosphorus pentachloride (12.2 g, 70 mmol) were added to 150 ml of toluene under a nitrogen atmosphere, and the mixture was stirred and refluxed. After 12 hours of reaction, cooling was performed to room temperature, and then the organic solvent was distilled under reduced pressure. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.1 g of Chemical Formula AA (yield 67%, MS: [M+H]+=260).

### Preparation Example 2: Synthesis of Chemical Formula AB

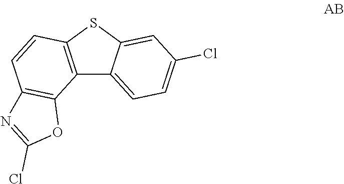 [SMILES: Clc1ccc2c(c1)sc1ccc3nc(Cl)oc3c12]

Chemical Formula AB was prepared in the same manner as in Preparation Example 1, except that (4-chloro-2-(methylthio)phenyl)boronic acid was used instead of (3-chloro-2-(methylthio)phenyl)boronic acid.

### Preparation Example 3: Synthesis of Chemical Formula AC

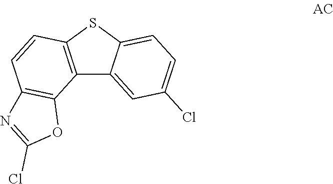 [SMILES: Clc1ccc2sc3ccc4nc(Cl)oc4c3c2c1]

Chemical Formula AC was prepared in the same manner as in Preparation Example 1, except that (5-chloro-2-(methylthio)phenyl)boronic acid was used instead of (3-chloro-2-(methylthio)phenyl)boronic acid.

### Preparation Example 4: Synthesis of Chemical Formula AD

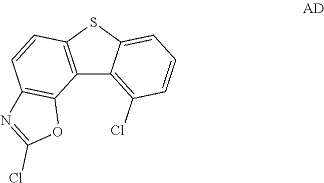 [SMILES: Clc1nc2ccc3sc4cccc(Cl)c4c3c2o1]

Chemical Formula AD was prepared in the same manner as in Preparation Example 1, except that (2-chloro-6-(methylthio)phenyl)boronic acid was used instead of (3-chloro-2-(methylthio)phenyl)boronic acid.

### Synthesis Scheme of Preparation Examples 5 to 6

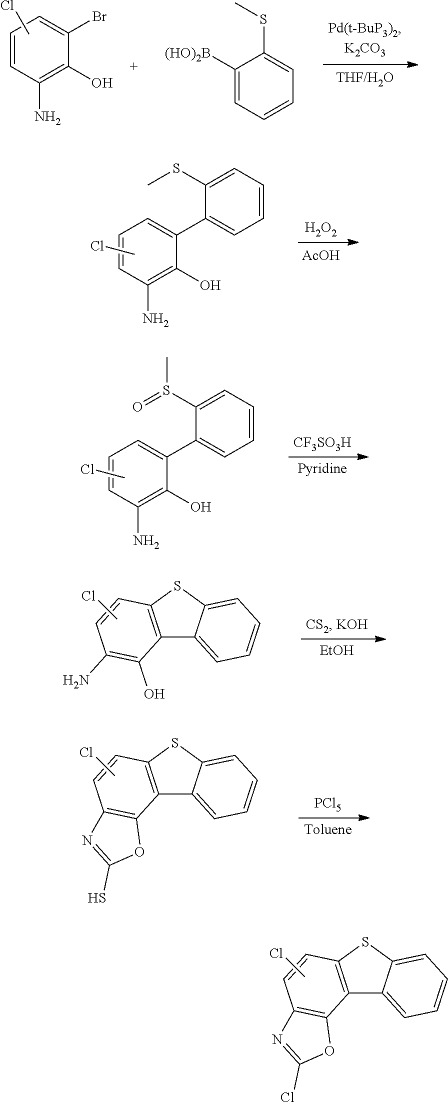

### Preparation Example 5: Synthesis of Chemical Formula AE

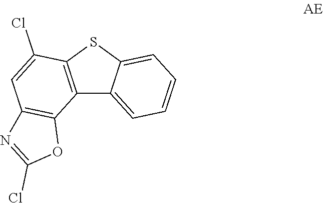 [SMILES: Clc1nc2cc(Cl)c3sc4ccccc4c3c2o1]

Chemical Formula AE was prepared in the same manner as in Preparation Example 1, except that 2-amino-6-bromo-4-chlorophenol was used instead of 2-amino-6-bromophenol and (2-(methylthio)phenyl)boronic acid was used instead of (3-chloro-2-(methylthio)phenyl)boronic acid.

### Preparation Example 6: Synthesis of Chemical Formula AF

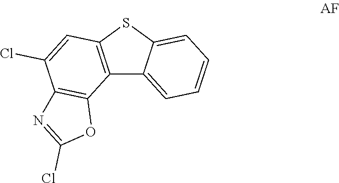 [SMILES: Clc1nc2c(Cl)cc3sc4ccccc4c3c2o1]

Chemical Formula AE was prepared in the same manner as in Preparation Example 1, except that 2-amino-6-bromo-3-chlorophenol was used instead of 2-amino-6-bromophenol and (2-(methylthio)phenyl)boronic acid was used instead of (3-chloro-2-(methylthio)phenyl)boronic acid.

### Synthesis Scheme of Preparation Examples 7 to 10

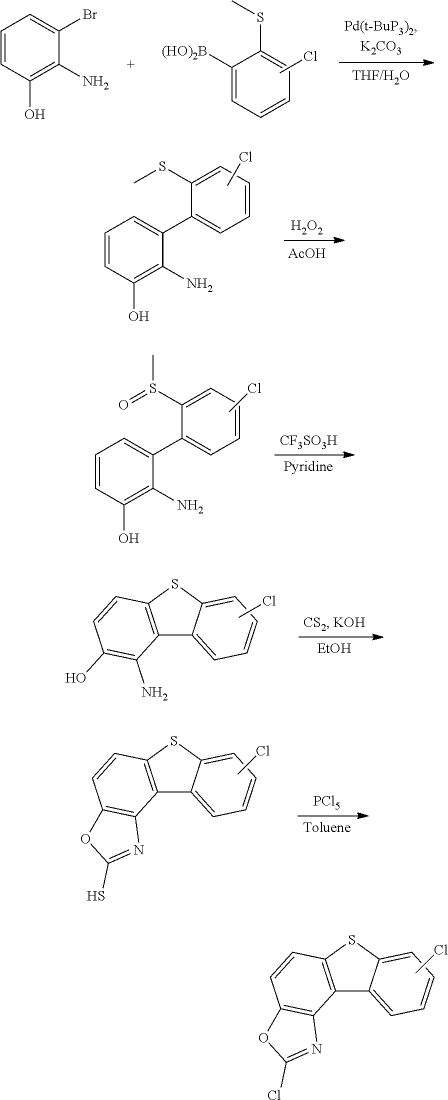

### Preparation Example 7: Synthesis of Chemical Formula BA

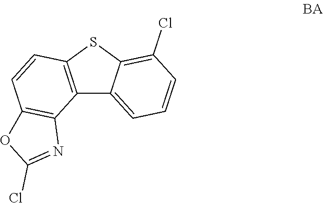 [SMILES: Clc1nc2c(ccc3sc4c(Cl)cccc4c32)o1]

Chemical Formula BA was prepared in the same manner as in Preparation Example 1, except that 2-amino-3-bromophenol was used instead of 2-amino-6-bromophenol.

### Preparation Example 8: Synthesis of Chemical Formula BB

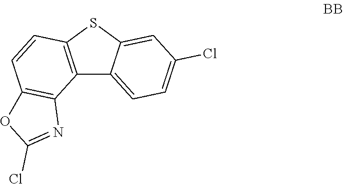 [SMILES: Clc1ccc2c(c1)sc1ccc3oc(Cl)nc3c12]

Chemical Formula BB was prepared in the same manner as in Preparation Example 1, except that 2-amino-3-bromophenol was used instead of 2-amino-6-bromophenol and (4-chloro-2-(methylthio)phenyl)boronic acid was used instead of (3-chloro-2-(methylthio)phenyl)boronic acid.

### Preparation Example 9: Synthesis of Chemical Formula BC

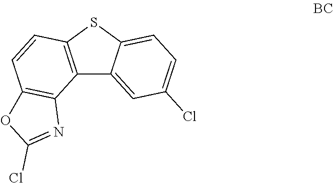 [SMILES: Clc1ccc2sc3ccc4oc(Cl)nc4c3c2c1]

Chemical Formula BC was prepared in the same manner as in Preparation Example 1, except that 2-amino-3-bromophenol was used instead of 2-amino-6-bromophenol and (5-chloro-2-(methylthio)phenyl)boronic acid was used instead of (3-chloro-2-(methylthio)phenyl)boronic acid.

### Preparation Example 10: Synthesis of Chemical Formula BD

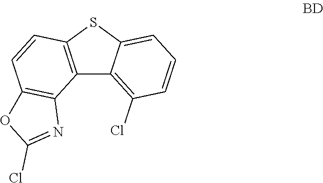 [SMILES: Clc1nc2c(ccc3sc4cccc(Cl)c4c32)o1]

Chemical Formula BD was prepared in the same manner as in Preparation Example 1, except that 2-amino-3-bromophenol was used instead of 2-amino-6-bromophenol and (2-chloro-6-(methylthio)phenyl)boronic acid was used instead of (3-chloro-2-(methylthio)phenyl)boronic acid.

### Synthesis Scheme of Preparation Examples 11 to 12

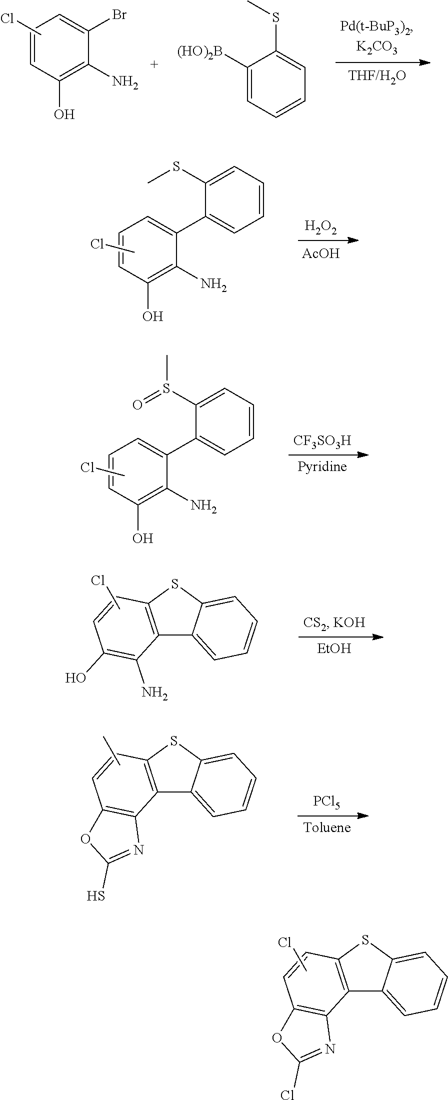

### Preparation Example 11: Synthesis of Chemical Formula BE

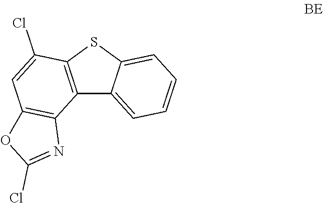 [SMILES: Clc1nc2c(cc(Cl)c3sc4ccccc4c32)o1]

Chemical Formula BE was prepared in the same manner as in Preparation Example 1, except that 2-amino-3-bromo-5-chlorophenol was used instead of 2-amino-6-bromophenol and (2-(methylthio)phenyl)boronic acid was used instead of (3-chloro-2-(methylthio)phenyl)boronic acid.

### Preparation Example 12: Synthesis of Chemical Formula BF

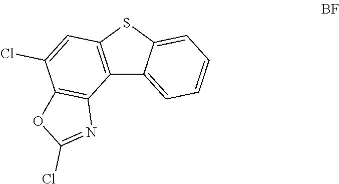 [SMILES: Clc1nc2c(o1)c(Cl)cc1sc3ccccc3c12]

Chemical Formula BF was prepared in the same manner as in Preparation Example 1, except that 2-amino-3-bromo-6-chlorophenol was used instead of 2-amino-6-bromophenol and (2-(methylthio)phenyl)boronic acid was used instead of (3-chloro-2-(methylthio)phenyl)boronic acid.

### Synthesis Scheme of Preparation Examples 13 to 16

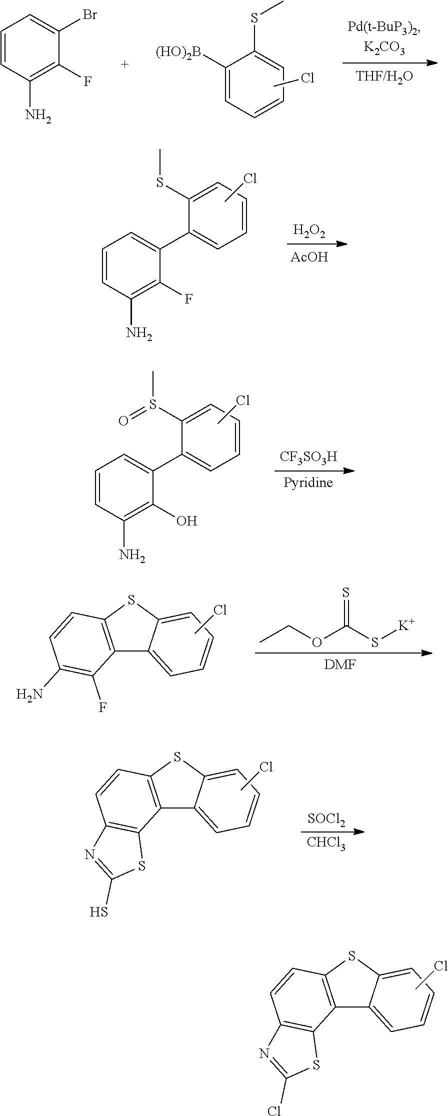

### Preparation Example 13: Synthesis of Chemical Formula CA

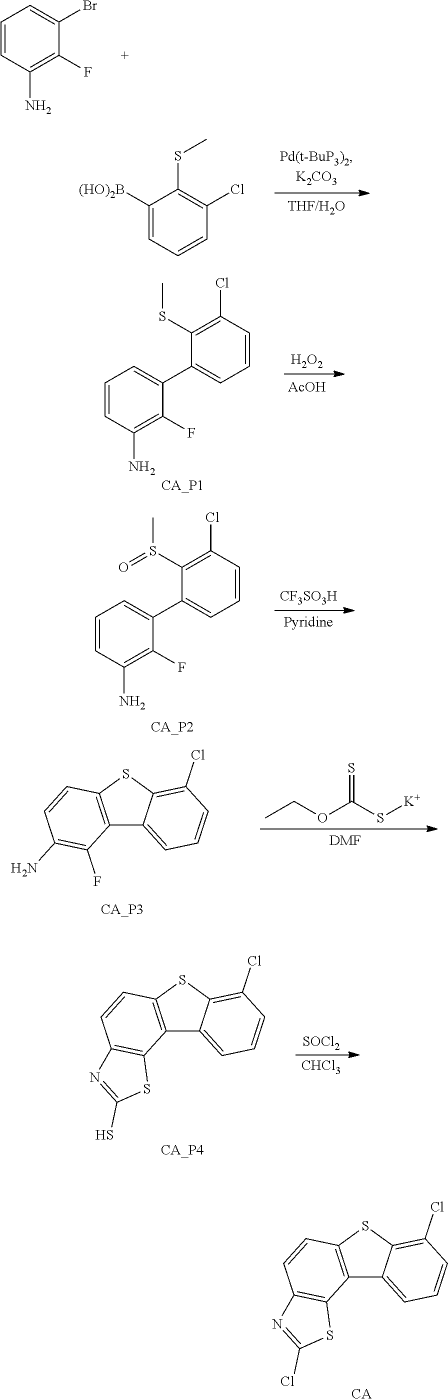

3-bromo-2-fluoroaniline (15 g, 78.9 mmol) and (3-chloro-2-(methylthio)phenyl)boronic acid (24 g, 118.4 mmol) were added to 300 ml of THF under a nitrogen atmosphere, and the mixture was stirred and refluxed. Then, potassium carbonate (32.7 g, 236.8 mmol) was dissolved in 98 ml of water, and then added thereto. Thereafter, it was stirred sufficiently, followed by adding bis(tri-tert-butylphosphine)palladium(0) (0.4 g, 0.8 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.8 g of Chemical Formula CA_P1 (yield 51%, MS: [M+H]+=268).

Chemical Formula CA_P1 (15 g, 56.2 mmol) and hydrogen peroxide (2.9 g, 84.3 mmol) were added to 300 ml of acetic acid under a nitrogen atmosphere, and the mixture was stirred and refluxed. After 10 hours of reaction, cooling was performed to room temperature, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 8.6 g of Chemical Formula CA_P2 (yield 54%, MS: [M+H]+=284).

Chemical Formula CA_P2 (15 g, 53 mmol) and trifluoromethanesulfonic acid (11.9 g, 79.5 mmol) were added to 300 ml of pyridine under a nitrogen atmosphere, and stirred at room temperature. After 11 hours of reaction, it was poured into 600 ml of water for solidification, and then filtered. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 6.9 g of Chemical Formula CA_P3 (yield 52%, MS: [M+H]+=252).

Chemical Formula CA_P3 (15 g, 59.7 mmol) and potassium O-ethyl dithiocarbonate (21.0 g, 131 mmol) were added to 150 ml of DMF under a nitrogen atmosphere, and the mixture was stirred and refluxed. After 9 hours of reaction, cooling was performed to room temperature, and then the organic solvent was distilled under reduced pressure. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.7 g of Chemical Formula CA_P4 (yield 80%, MS: [M+H]+=308).

Chemical Formula CA_P4 (15 g, 48.7 mmol) was added to 150 ml of CHCl₃ under a nitrogen atmosphere, and cooled to 0° C. with an ice bath. Then, thionyl chloride (12.8 g, 107.5 mmol) was slowly added dropwise, followed by stirring. After 4 hours of reaction, cooling was performed to room temperature, and then the organic solvent was distilled under reduced pressure. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.3 g of Chemical Formula CA (yield 68%, MS: [M+H]+=310).

### Preparation Example 14: Synthesis of Chemical Formula CB

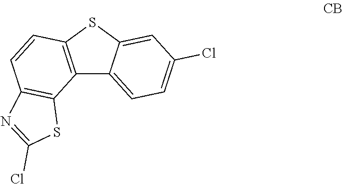

Chemical Formula CB was prepared in the same manner as in Preparation Example 13, except that (4-chloro-2-(methylthio)phenyl)boronic acid was used instead of (3-chloro-2-(methylthio)phenyl)boronic acid.

### Preparation Example 15: Synthesis of Chemical Formula CC

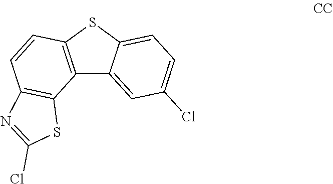

Chemical Formula CC was prepared in the same manner as in Preparation Example 13, except that (5-chloro-2-(methylthio)phenyl)boronic acid was used instead of (3-chloro-2-(methylthio)phenyl)boronic acid.

### Preparation Example 16: Synthesis of Chemical Formula CD

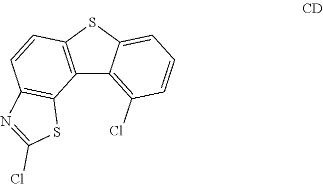

Chemical Formula CD was prepared in the same manner as in Preparation Example 13, except that (2-chloro-6-(methylthio)phenyl)boronic acid was used instead of (3-chloro-2-(methylthio)phenyl)boronic acid.

### Synthesis Scheme of Preparation Examples 17 to 18

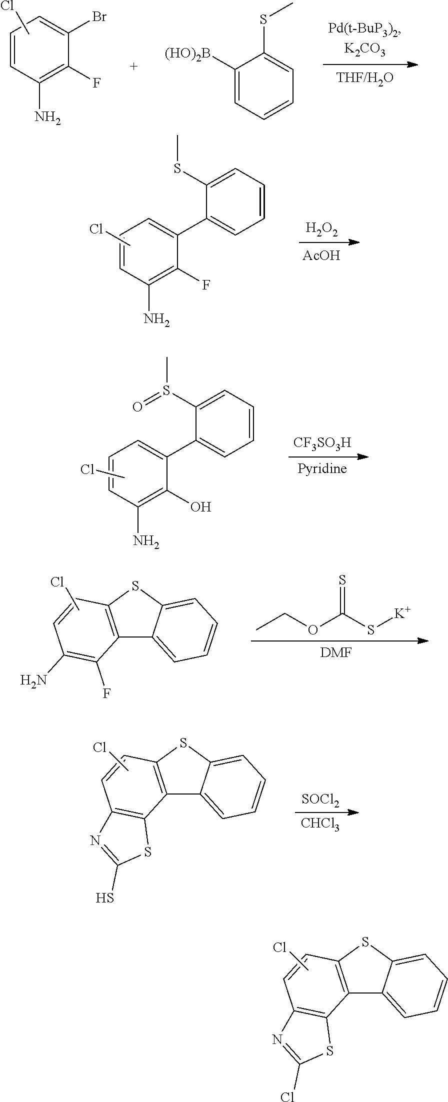

### Preparation Example 17: Synthesis of Chemical Formula CE

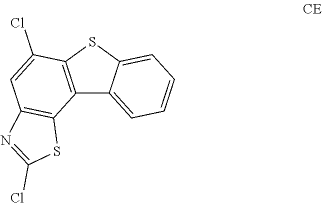

Chemical Formula CE was prepared in the same manner as in Preparation Example 13, except that 3-bromo-5-chloro-2-fluoroaniline was used instead of 3-bromo-2-fluoroaniline and (2-(methylthio)phenyl)boronic acid was used instead of (3-chloro-2-(methylthio)phenyl)boronic acid.

### Preparation Example 18: Synthesis of Chemical Formula CF

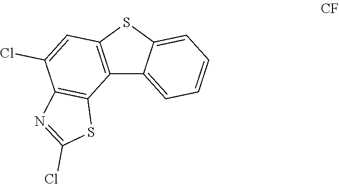

Chemical Formula CF was prepared in the same manner as in Preparation Example 13, except that 3-bromo-6-chloro-2-fluoroaniline was used instead of 3-bromo-2-fluoroaniline and (2-(methylthio)phenyl)boronic acid was used instead of (3-chloro-2-(methylthio)phenyl)boronic acid.

### Synthesis Scheme of Preparation Examples 19 to 22

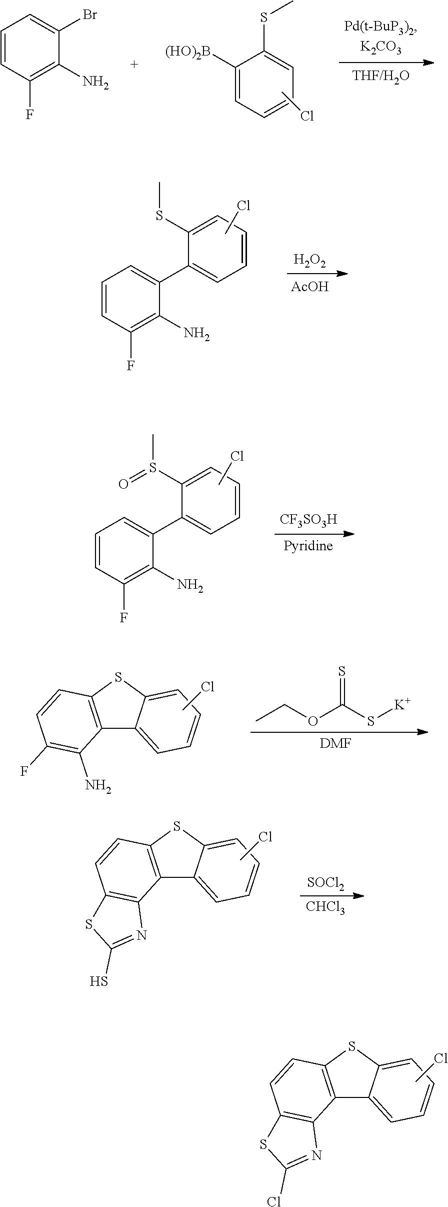

### Preparation Example 19: Synthesis of Chemical Formula DA

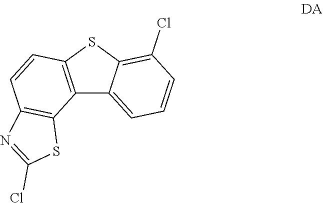

Chemical Formula DA was prepared in the same manner as in Preparation Example 13, except that 2-bromo-6-fluoroaniline was used instead of 3-bromo-2-fluoroaniline.

### Preparation Example 20: Synthesis of Chemical Formula DB

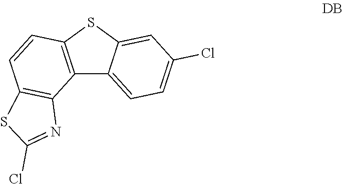 [SMILES: Clc1ccc2c(c1)sc1ccc3sc(Cl)nc3c12]

Chemical Formula DB was prepared in the same manner as in Preparation Example 13, except that 2-bromo-6-fluoroaniline was used instead of 3-bromo-2-fluoroaniline and (4-chloro-2-(methylthio)phenyl)boronic acid was used instead of (3-chloro-2-(methylthio)phenyl)boronic acid.

### Preparation Example 21: Synthesis of Chemical Formula DC

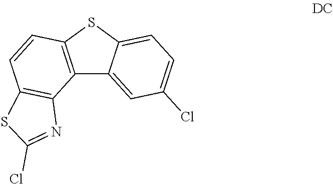 [SMILES: Clc1ccc2sc3ccc4sc(Cl)nc4c3c2c1]

Chemical Formula DC was prepared in the same manner as in Preparation Example 13, except that 2-bromo-6-fluoroaniline was used instead of 3-bromo-2-fluoroaniline and (5-chloro-2-(methylthio)phenyl)boronic acid was used instead of (3-chloro-2-(methylthio)phenyl)boronic acid.

### Preparation Example 22: Synthesis of Chemical Formula DD

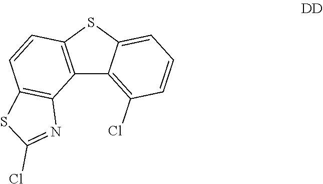 [SMILES: Clc1nc2c(ccc3sc4cccc(Cl)c4c32)s1]

Chemical Formula DD was prepared in the same manner as in Preparation Example 13, except that 2-bromo-6-fluoroaniline was used instead of 3-bromo-2-fluoroaniline and (2-chloro-6-(methylthio)phenyl)boronic acid was used instead of (3-chloro-2-(methylthio)phenyl)boronic acid.

### Synthesis Scheme of Preparation Examples 23 to 24

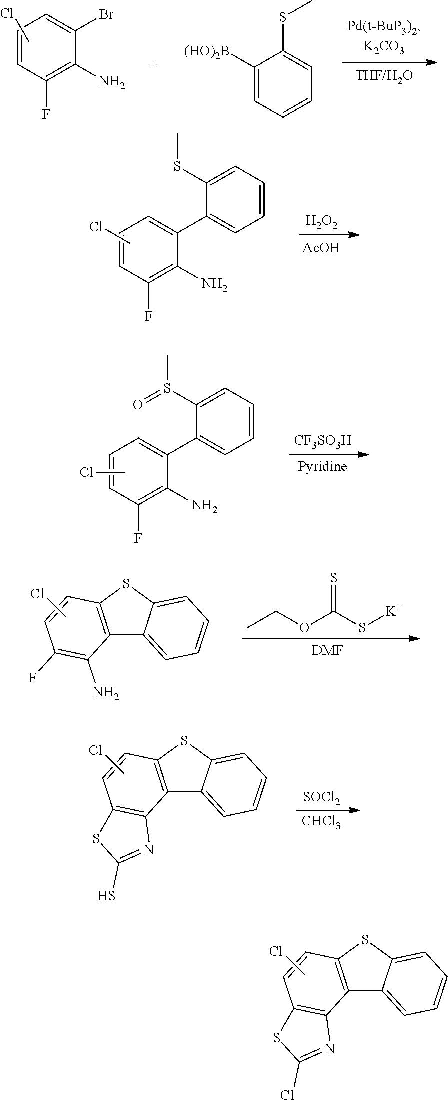

### Preparation Example 23: Synthesis of Chemical Formula DE

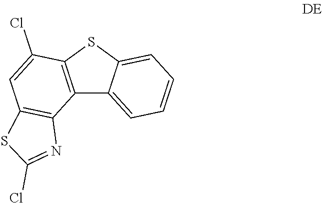 [SMILES: Clc1nc2c(cc(Cl)c3sc4ccccc4c32)s1]

Chemical Formula DE was prepared in the same manner as in Preparation Example 13, except that 2-bromo-4-chloro-6-fluoroaniline was used instead of 3-bromo-2-fluoroaniline and (2-(methylthio)phenyl)boronic acid was used instead of (3-chloro-2-(methylthio)phenyl)boronic acid.

### Preparation Example 24: Synthesis of Chemical Formula DF

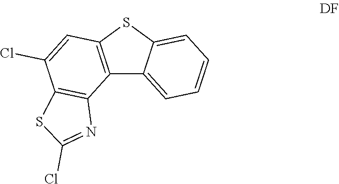 [SMILES: Clc1nc2c(s1)c(Cl)cc1sc3ccccc3c12]

Chemical Formula DF was prepared in the same manner as in Preparation Example 13, except that 6-bromo-3-chloro-2-fluoroaniline was used instead of 4-bromo-2-fluoroaniline and (2-(methylthio)phenyl)boronic acid was used instead of (3-chloro-2-(methylthio)phenyl)boronic acid.

### Synthesis Examples: Preparation of Compound of Chemical Formula 1

### Synthesis Example 1-1

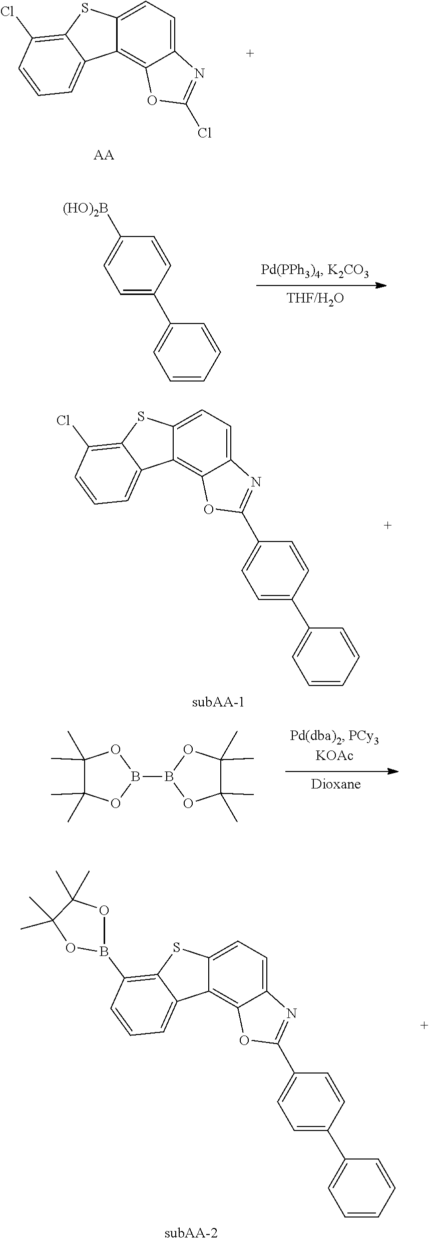

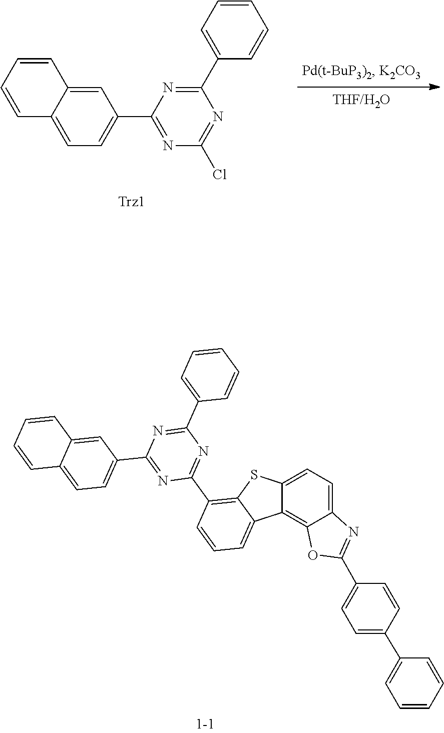 [SMILES: Clc1nc(-c2ccccc2)nc(-c2ccc3ccccc3c2)n1 ; c1ccc(-c2ccc(-c3nc4ccc5sc6c(-c7nc(-c8ccccc8)nc(-c8ccc9ccccc9c8)n7)cccc6c5c4o3)cc2)cc1]

Chemical Formula AA (15 g, 51 mmol) and [1,1′-biphenyl]-4-ylboronic acid (10.6 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.8 g of subAA-1 (yield 61%, MS: [M+H]+=412).

subAA-1 (15 g, 36.4 mmol) and bis(pinacolato)diboron (10.2 g, 40.1 mmol) were added to 300 ml of 1,4-dioxane under a nitrogen atmosphere, and the mixture was stirred and refluxed. Then, potassium acetate (5.4 g, 54.6 mmol) was added and stirred sufficiently, followed by adding bis(dibenzylideneacetone)palladium(0) (0.6 g, 1.1 mmol) and tricyclohexylphosphine (0.6 g, 2.2 mmol). After 7 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.5 g of subAA-2 (yield 63%, MS: [M+H]+=504).

subAA-2 (15 g, 29.8 mmol) and Trz1 (9.9 g, 31.3 mmol) were added to 300 ml of THF under a nitrogen atmosphere, and the mixture was stirred and refluxed. Then, potassium carbonate (12.4 g, 89.4 mmol) was dissolved in 37 ml of water, and then added thereto. Thereafter, it was stirred sufficiently, followed by adding bis(tri-tert-butylphosphine)palladium(0) (0.2 g, 0.3 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.7 g of Compound 1-1 (yield 70%, MS: [M+H]+=659).

### Synthesis Example 1-2

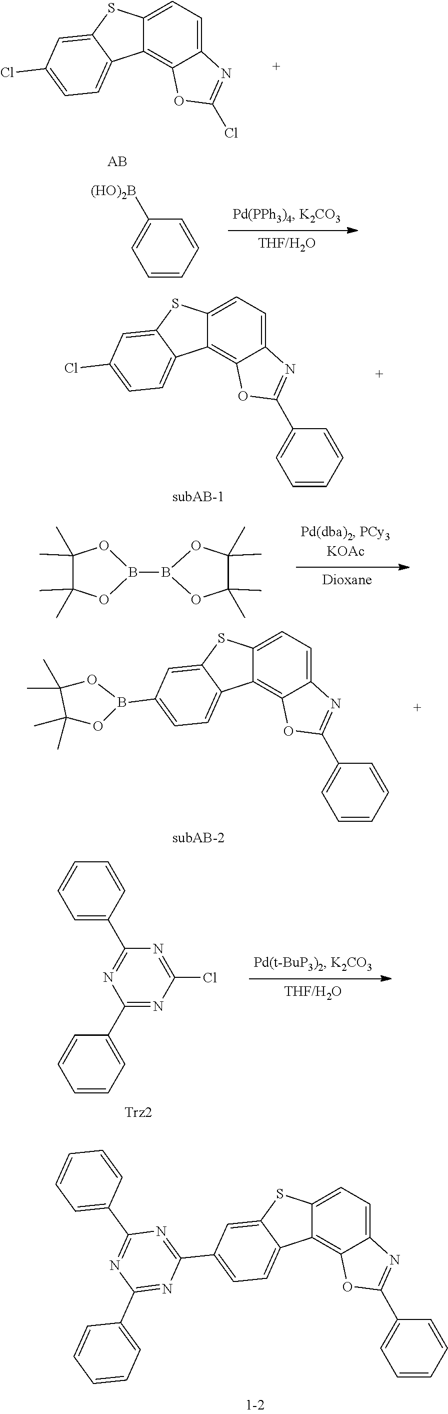

Chemical Formula AB (15 g, 51 mmol) and phenylboronic acid (6.5 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.6 g of subAB-1 (yield 56%, MS: [M+H]+=336).

subAB-1 (15 g, 44.7 mmol) and bis(pinacolato)diboron (12.5 g, 49.1 mmol) were added to 300 ml of 1,4-dioxane under a nitrogen atmosphere, and the mixture was stirred and refluxed. Then, potassium acetate (6.6 g, 67 mmol) was added and stirred sufficiently, followed by adding bis(dibenzylideneacetone)palladium(0) (0.8 g, 1.3 mmol) and tricyclohexylphosphine (0.8 g, 2.7 mmol). After 6 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.6 g of subAB-2 (yield 66%, MS: [M+H]+=428).

subAB-2 (15 g, 35.1 mmol) and Trz2 (9.9 g, 36.9 mmol) were added to 300 ml of THF under a nitrogen atmosphere, and the mixture was stirred and refluxed. Then, potassium carbonate (14.6 g, 105.3 mmol) was dissolved in 44 ml of water, and then added thereto. Thereafter, it was stirred sufficiently, followed by adding bis(tri-tert-butylphosphine)palladium(0) (0.2 g, 0.4 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12 g of Compound 1-2 (yield 64%, MS: [M+H]+=533).

### Synthesis Example 1-3

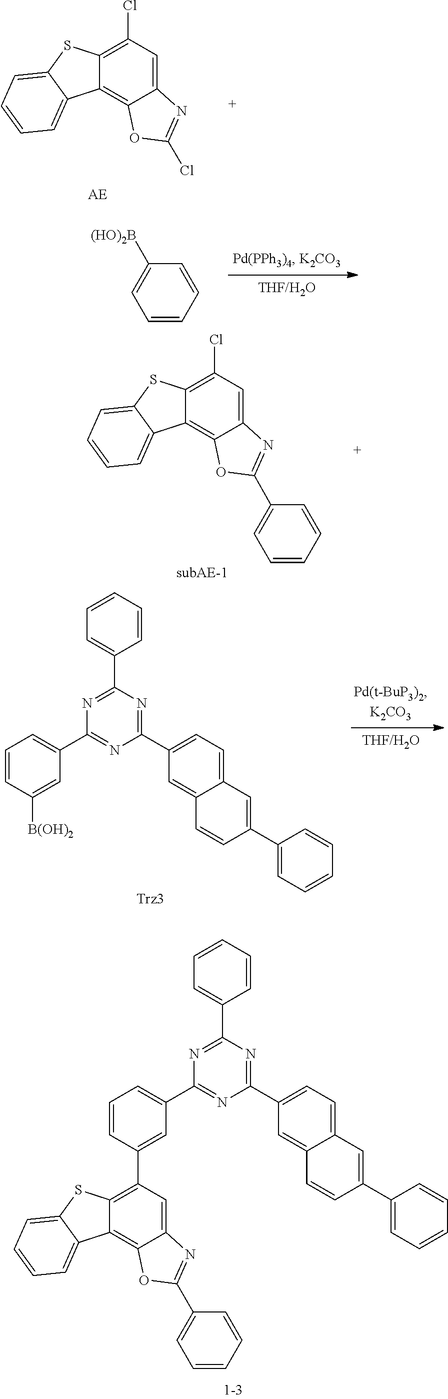

Chemical Formula AE (15 g, 51 mmol) and phenylboronic acid (6.5 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.4 g of subAE-1 (yield 55%, MS: [M+H]+=336).

subAE-1 (15 g, 44.7 mmol) and Trz3 (22.5 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20 g of Compound 1-3 (yield 61%, MS: [M+H]+=735).

### Synthesis Example 1-4

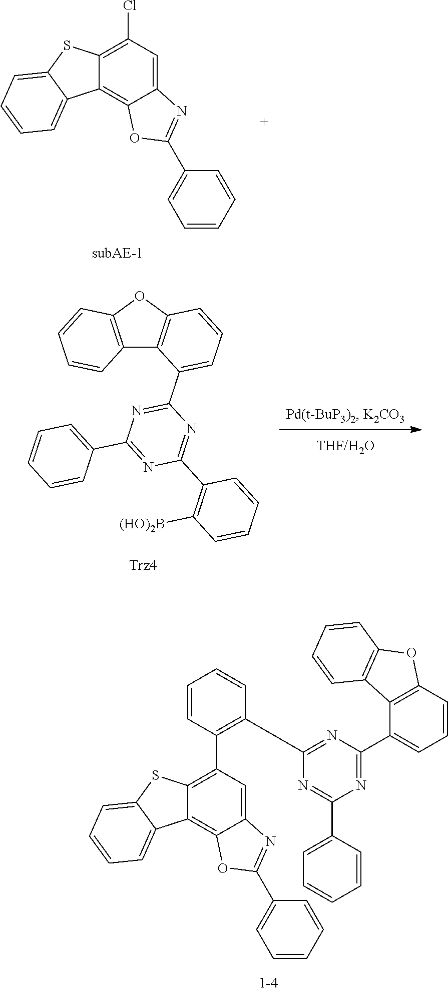 [SMILES: Clc1cc2nc(-c3ccccc3)oc2c2c1sc1ccccc12 ; OB(O)c1ccccc1-c1nc(-c2ccccc2)nc(-c2cccc3oc4ccccc4c23)n1 ; c1ccc(-c2nc(Cc3ccccc3-c3cc4nc(-c5ccccc5)oc4c4c3sc3ccccc34)nc(-c3cccc4oc5ccccc5c34)n2)cc1]

subAE-1 (15 g, 44.7 mmol) and Trz4 (20.8 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16.8 g of Compound 1-4 (yield 54%, MS: [M+H]+=699).

### Synthesis Example 1-5

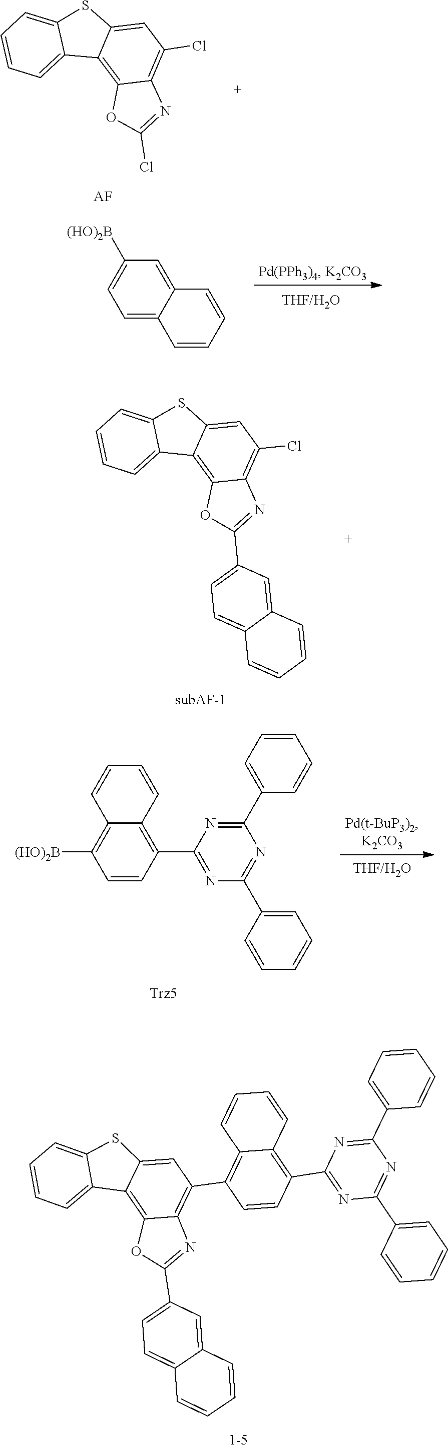

Chemical Formula AF (15 g, 51 mmol) and naphthalen-2-ylboronic acid (9.2 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.5 g of subAF-1 (yield 69%, MS: [M+H]+=386).

subAF-1 (15 g, 38.9 mmol) and Trz5 (16.5 g, 40.8 mmol) were added to 300 ml of THF under a nitrogen atmosphere, and the mixture was stirred and refluxed. Then, potassium carbonate (16.1 g, 116.6 mmol) was dissolved in 48 ml of water, and then added thereto. Thereafter, it was stirred sufficiently, followed by adding bis(tri-tert-butylphosphine)palladium(0) (0.2 g, 0.4 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.2 g of Compound 1-5 (yield 66%, MS: [M+H]+=709).

### Synthesis Example 1-6

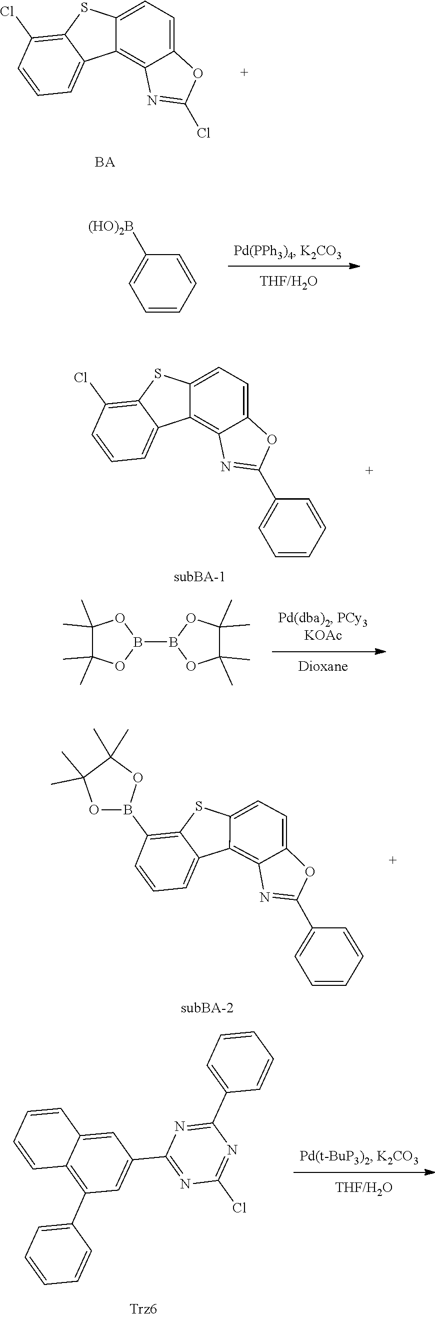

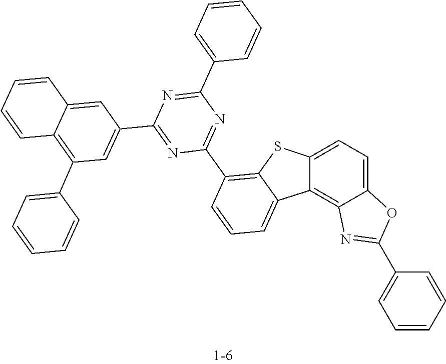 [SMILES: c1ccc(-c2nc(-c3cc(-c4ccccc4)c4ccccc4c3)nc(-c3cccc4c3sc3ccc5oc(-c6ccccc6)nc5c34)n2)cc1]

Chemical Formula BA (15 g, 51 mmol) and phenylboronic acid (6.5 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.8 g of subBA-1 (yield 69%, MS: [M+H]+=336).

subBA-1 (15 g, 44.7 mmol) and bis(pinacolato)diboron (12.5 g, 49.1 mmol) were added to 300 ml of 1,4-dioxane under a nitrogen atmosphere, and the mixture was stirred and refluxed. Then, potassium acetate (6.6 g, 67 mmol) was added and stirred sufficiently, followed by adding bis(dibenzylideneacetone)palladium(0) (0.8 g, 1.3 mmol) and tricyclohexylphosphine (0.8 g, 2.7 mmol). After 8 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.9 g of subBA-2 (yield 78%, MS: [M+H]+=428).

subBA-2 (15 g, 35.1 mmol) and Trz6 (14.5 g, 36.9 mmol) were added to 300 ml of THF under a nitrogen atmosphere, and the mixture was stirred and refluxed. Then, potassium carbonate (14.6 g, 105.3 mmol) was dissolved in 44 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.6 g of Compound 1-6 (yield 59%, MS: [M+H]+=659).

### Synthesis Example 1-7

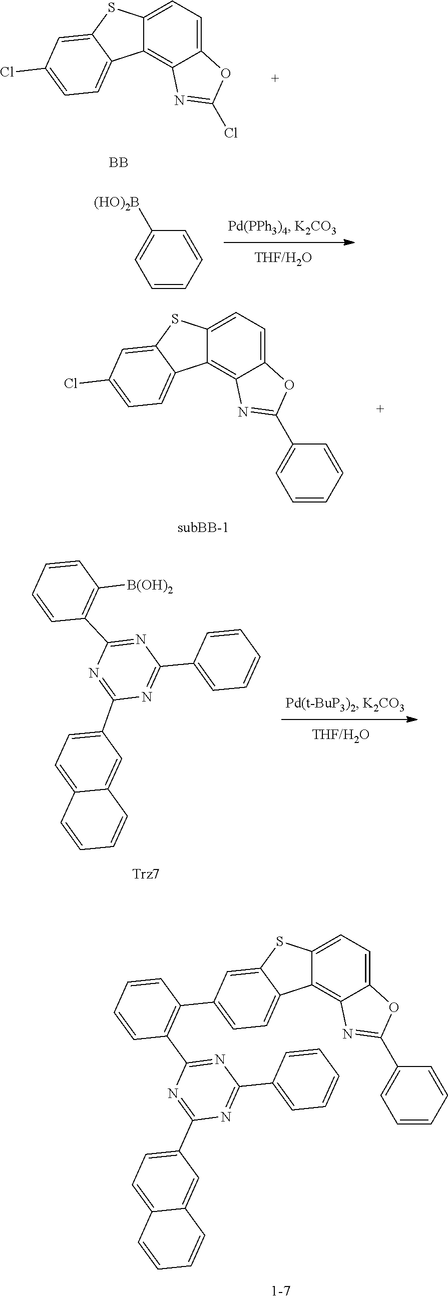

Chemical Formula BB (15 g, 51 mmol) and phenylboronic acid (6.5 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.3 g of subBB-1 (yield 60%, MS: [M+H]+=336).

subBB-1 (15 g, 44.7 mmol) and Trz7 (18.9 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.9 g of Compound 1-7 (yield 61%, MS: [M+H]+=659).

### Synthesis Example 1-8

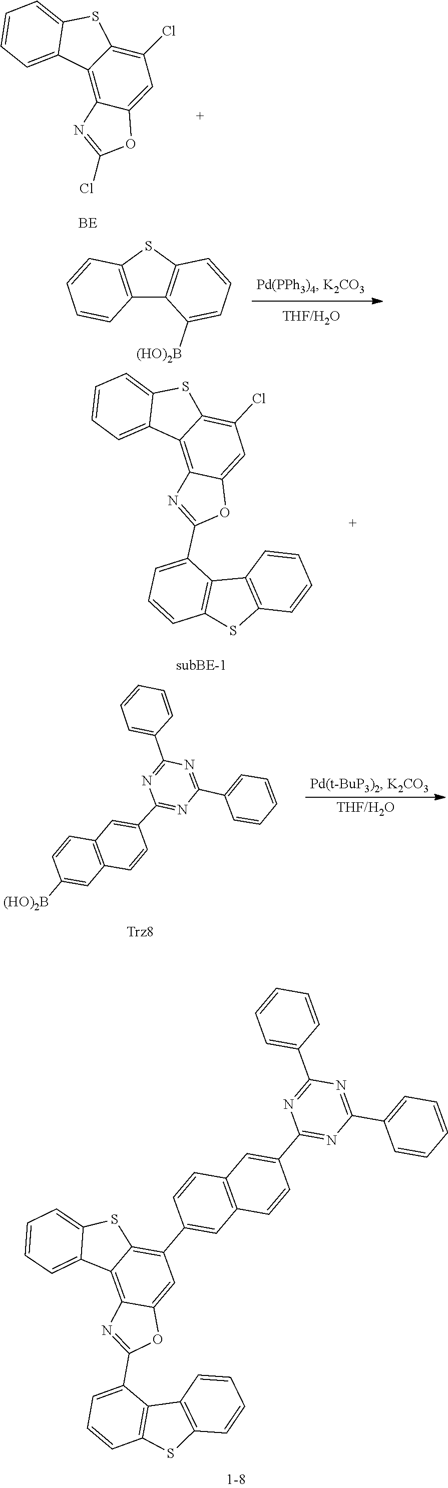

Chemical Formula BE (15 g, 51 mmol) and dibenzo[b,d]thiophen-1-ylboronic acid (12.2 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.7 g of subBE-1 (yield 52%, MS: [M+H]+=442).

subBE-1 (15 g, 33.9 mmol) and Trz8 (14.4 g, 35.6 mmol) were added to 300 ml of THF under a nitrogen atmosphere, and the mixture was stirred and refluxed. Then, potassium carbonate (14.1 g, 101.8 mmol) was dissolved in 42 ml of water, and then added thereto. Thereafter, it was stirred sufficiently, followed by adding bis(tri-tert-butylphosphine)palladium(0) (0.2 g, 0.3 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.8 g of Compound 1-8 (yield 57%, MS: [M+H]+=765).

### Synthesis Example 1-9

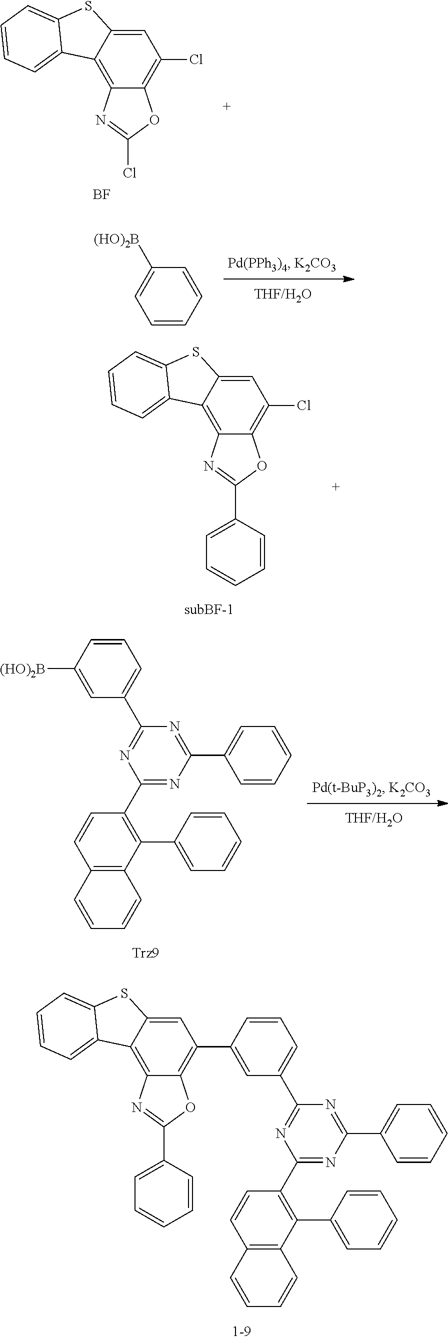

Chemical Formula BF (15 g, 51 mmol) and phenylboronic acid (6.5 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.3 g of subBF-1 (yield 66%, MS: [M+H]+=336).

subBF-1 (15 g, 44.7 mmol) and Trz9 (22.5 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16.7 g of Compound 1-9 (yield 51%, MS: [M+H]+=735).

### Synthesis Example 1-10

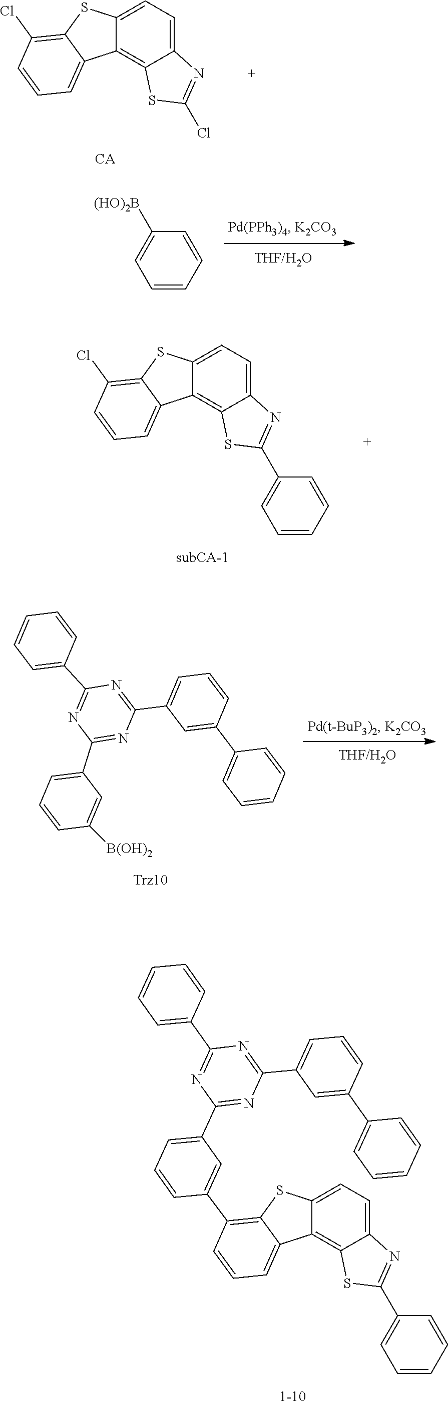 [SMILES: Clc1nc2ccc3sc4c(Cl)cccc4c3c2s1 ; OB(O)c1ccccc1 ; Clc1cccc2c1sc1ccc3nc(-c4ccccc4)sc3c12 ; OB(O)c1cccc(-c2nc(-c3ccccc3)nc(-c3cccc(-c4ccccc4)c3)n2)c1 ; c1ccc(-c2cccc(-c3nc(-c4ccccc4)nc(-c4cccc(-c5cccc6c5sc5ccc7nc(-c8ccccc8)sc7c56)c4)n3)c2)cc1]

Chemical Formula CA (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.3 g of subCA-1 (yield 55%, MS: [M+H]+=352).

subCA-1 (15 g, 42.6 mmol) and Trz10 (19.2 g, 44.8 mmol) were added to 300 ml of THF under a nitrogen atmosphere, and the mixture was stirred and refluxed. Then, potassium carbonate (17.7 g, 127.9 mmol) was dissolved in 53 ml of water, and then added thereto. Thereafter, it was stirred sufficiently, followed by adding bis(tri-tert-butylphosphine)palladium(0) (0.2 g, 0.4 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.6 g of Compound 1-10 (yield 69%, MS: [M+H]+=701).

### Synthesis Example 1-11

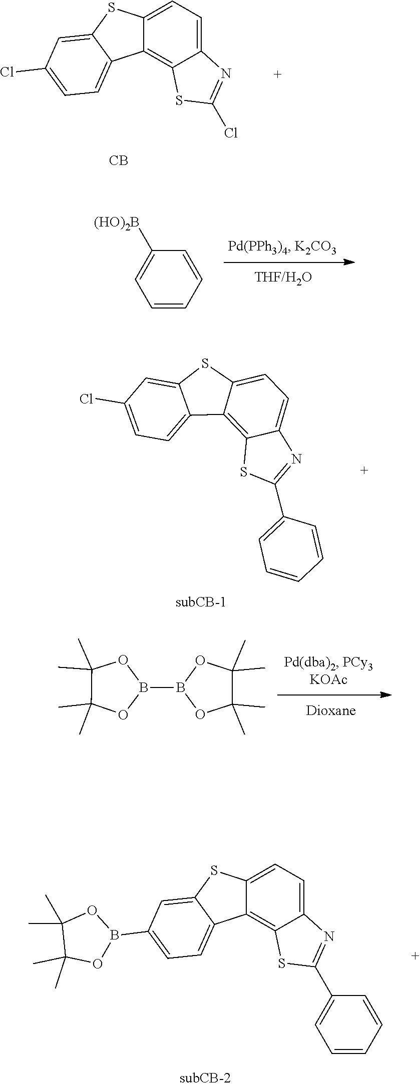

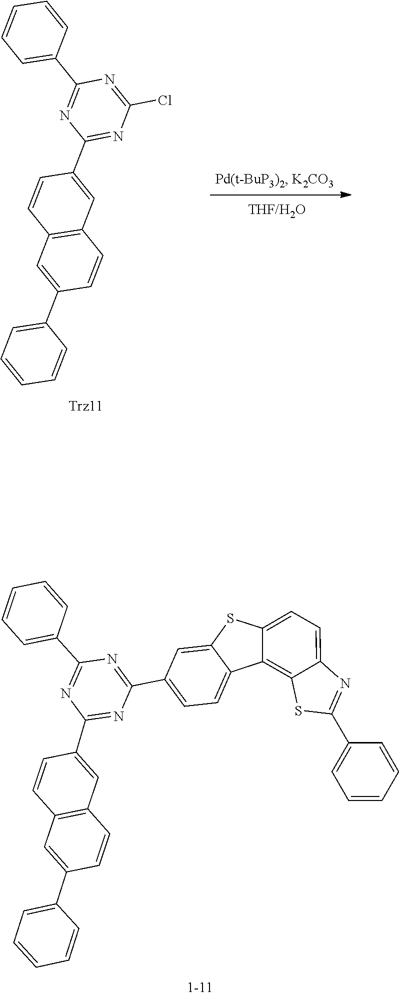 [SMILES: Clc1nc(-c2ccccc2)nc(-c2ccc3cc(-c4ccccc4)ccc3c2)n1 ; c1ccc(-c2ccc3cc(-c4nc(-c5ccccc5)nc(-c5ccc6c(c5)sc5ccc7nc(-c8ccccc8)sc7c56)n4)ccc3c2)cc1]

Chemical Formula CB (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.2 g of subCB-1 (yield 54%, MS: [M+H]+=352).

subCB-1 (15 g, 42.6 mmol) and bis(pinacolato)diboron (11.9 g, 46.9 mmol) were added to 300 ml of 1,4-dioxane under a nitrogen atmosphere, and the mixture was stirred and refluxed. Then, potassium acetate (6.3 g, 63.9 mmol) was added and stirred sufficiently, followed by adding bis(dibenzylideneacetone)palladium(0) (0.7 g, 1.3 mmol) and tricyclohexylphosphine (0.7 g, 2.6 mmol). After 5 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.2 g of subCB-2 (yield 75%, MS: [M+H]+=444).

subCB-2 (15 g, 33.8 mmol) and Trz11 (14 g, 35.5 mmol) were added to 300 ml of THF under a nitrogen atmosphere, and the mixture was stirred and refluxed. Then, potassium carbonate (14 g, 101.5 mmol) was dissolved in 42 ml of water, and then added thereto. Thereafter, it was stirred sufficiently, followed by adding bis(tri-tert-butylphosphine)palladium(0) (0.2 g, 0.3 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.2 g of Compound 1-11 (yield 58%, MS: [M+H]+=675).

### Synthesis Example 1-12

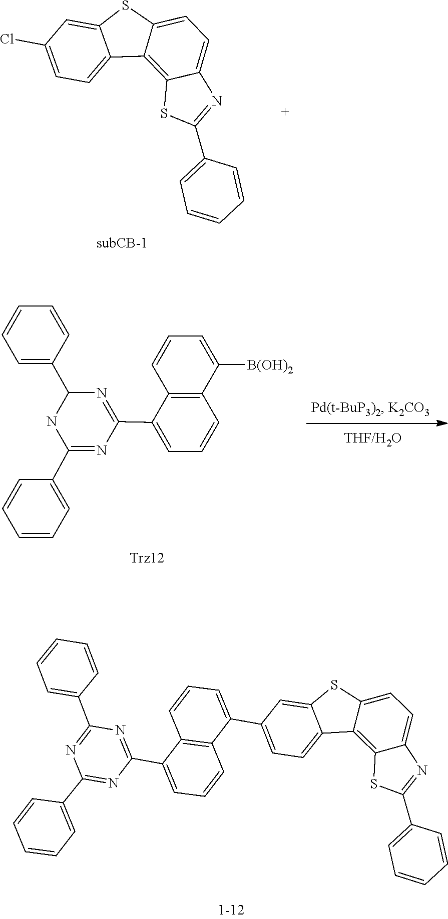

subCB-1 (15 g, 42.6 mmol) and Trz12 (18 g, 44.8 mmol) were added to 300 ml of THF under a nitrogen atmosphere, and the mixture was stirred and refluxed. Then, potassium carbonate (17.7 g, 127.9 mmol) was dissolved in 53 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.8 g of Compound 1-12 (yield 55%, MS: [M+H]+=675).

### Synthesis Example 1-13

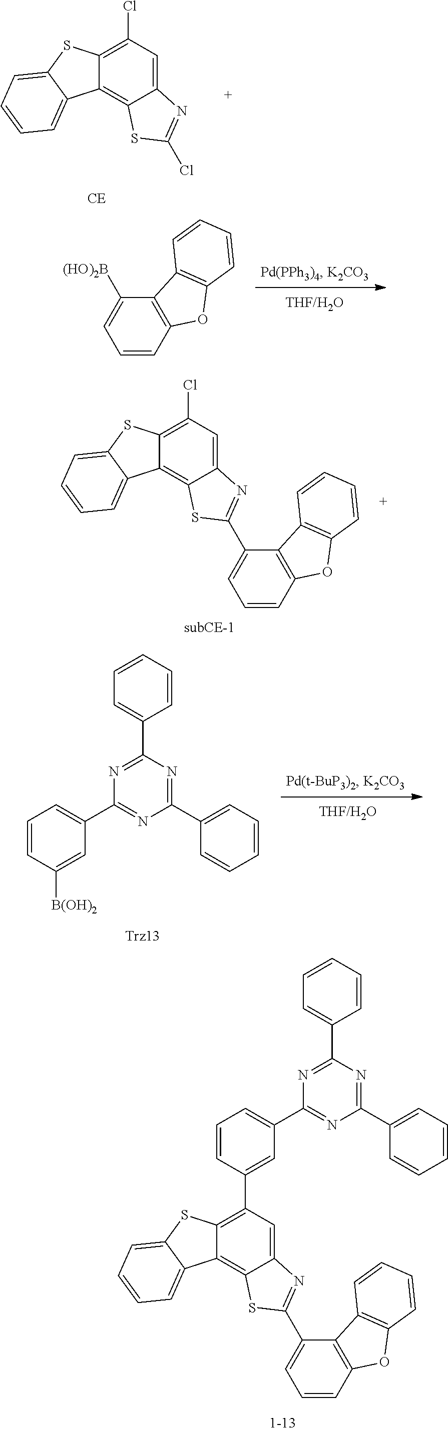

Chemical Formula CE (15 g, 48.4 mmol) and dibenzo[b,d]furan-1-ylboronic acid (10.8 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.4 g of subCE-1 (yield 58%, MS: [M+H]+=442).

subCE-1 (15 g, 33.9 mmol) and Trz13 (12.6 g, 35.6 mmol) were added to 300 ml of THF under a nitrogen atmosphere, and the mixture was stirred and refluxed. Then, potassium carbonate (14.1 g, 101.8 mmol) was dissolved in 42 ml of water, and then added thereto. Thereafter, it was stirred sufficiently, followed by adding bis(tri-tert-butylphosphine)palladium(0) (0.2 g, 0.3 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.5 g of Compound 1-13 (yield 64%, MS: [M+H]+=715).

### Synthesis Example 1-14

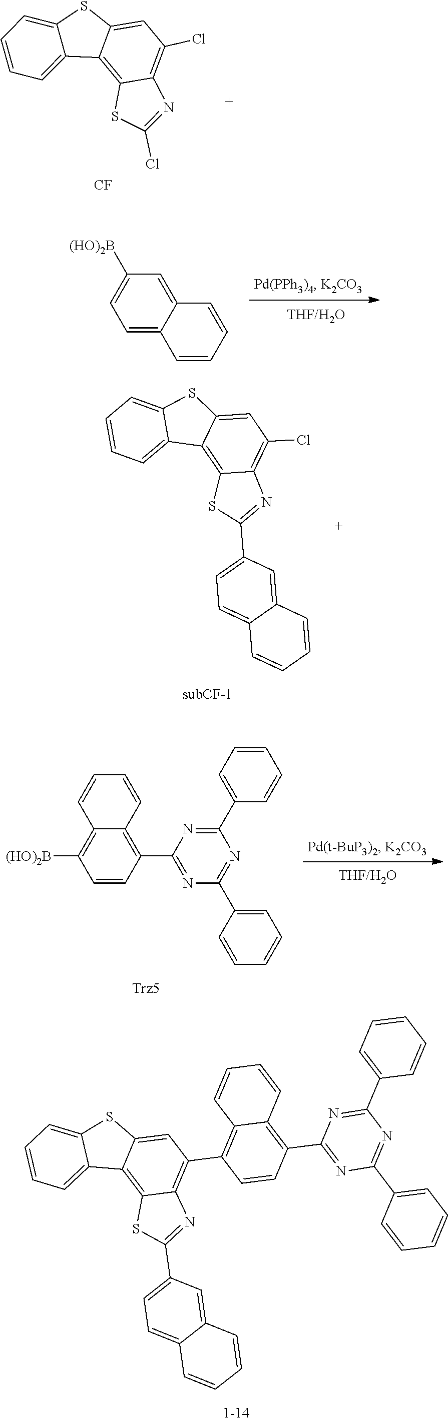

Chemical Formula CF (15 g, 48.4 mmol) and naphthalen-2-ylboronic acid (10.8 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13 g of subCF-1 (yield 67%, MS: [M+H]+=402).

subCF-1 (15 g, 37.3 mmol) and Trz5 (15.8 g, 39.2 mmol) were added to 300 ml of THF under a nitrogen atmosphere, and the mixture was stirred and refluxed. Then, potassium carbonate (15.5 g, 112 mmol) was dissolved in 46 ml of water, and then added thereto. Thereafter, it was stirred sufficiently, followed by adding bis(tri-tert-butylphosphine)palladium(0) (0.2 g, 0.4 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.4 g of Compound 1-14 (yield 68%, MS: [M+H]+=725).

### Synthesis Example 1-15

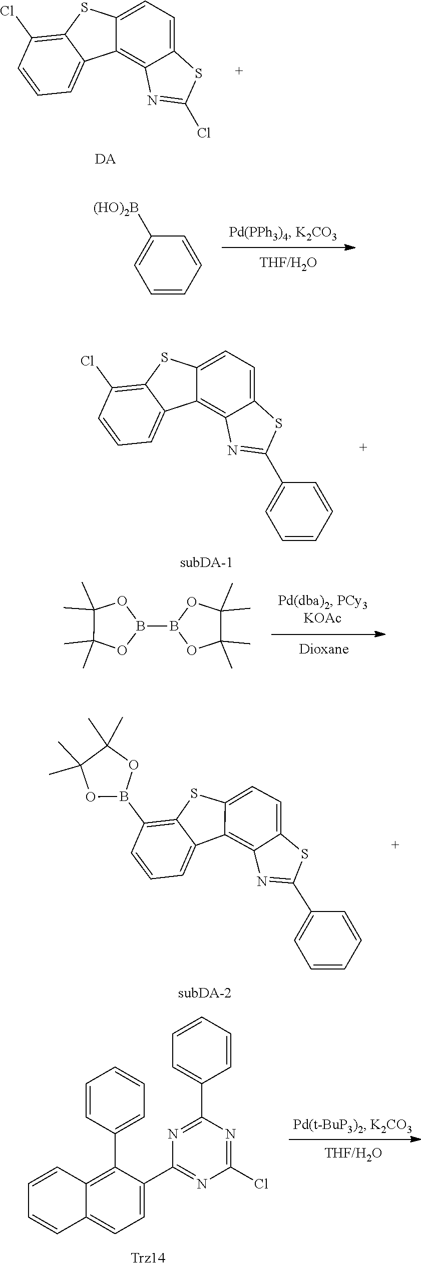

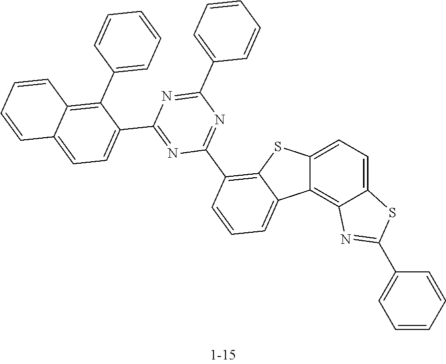 [SMILES: c1ccc(-c2nc(-c3ccc4ccccc4c3-c3ccccc3)nc(-c3cccc4c3sc3ccc5sc(-c6ccccc6)nc5c34)n2)cc1]

Chemical Formula DA (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.7 g of subDA-1 (yield 69%, MS: [M+H]+=352).

subDA-1 (15 g, 42.6 mmol) and bis(pinacolato)diboron (11.9 g, 46.9 mmol) were added to 300 ml of 1,4-dioxane under a nitrogen atmosphere, and the mixture was stirred and refluxed. Then, potassium acetate (6.3 g, 63.9 mmol) was added and stirred sufficiently, followed by adding bis(dibenzylideneacetone)palladium(0) (0.7 g, 1.3 mmol) and tricyclohexylphosphine (0.7 g, 2.6 mmol). After 9 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.7 g of subDA-2 (yield 78%, MS: [M+H]+=444).

subDA-2 (15 g, 33.8 mmol) and Trz14 (14 g, 35.5 mmol) were added to 300 ml of THF under a nitrogen atmosphere, and the mixture was stirred and refluxed. Then, potassium carbonate (14 g, 101.5 mmol) was dissolved in 42 ml of water, and then added thereto. Thereafter, it was stirred sufficiently, followed by adding bis(tri-tert-butylphosphine)palladium(0) (0.2 g, 0.3 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.7 g of Compound 1-15 (yield 69%, MS: [M+H]+=675).

### Synthesis Example 1-16

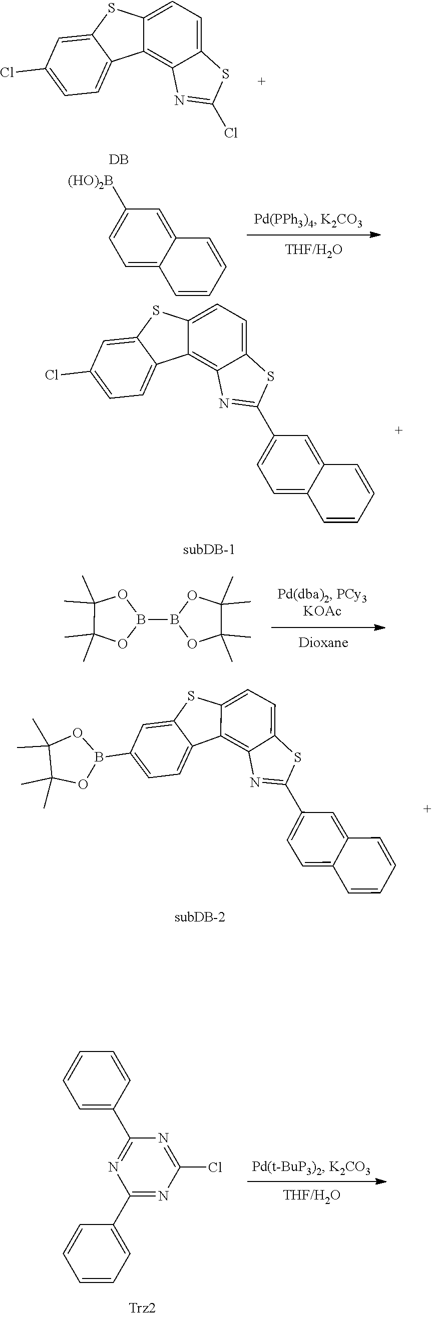

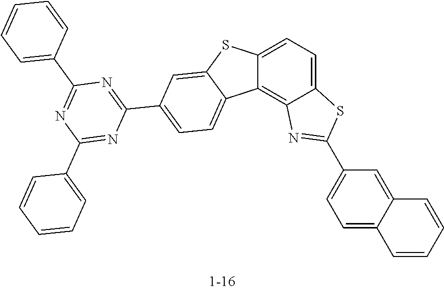 [SMILES: c1ccc(-c2nc(-c3ccccc3)nc(-c3ccc4c(c3)sc3ccc5sc(-c6ccc7ccccc7c6)nc5c34)n2)cc1]

Chemical Formula DB (15 g, 48.4 mmol) and naphthalen-2-ylboronic acid (10.8 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.9 g of subDB-1 (yield 51%, MS: [M+H]+=402).

subDB-1 (15 g, 37.3 mmol) and bis(pinacolato)diboron (10.4 g, 41.1 mmol) were added to 300 ml of 1,4-dioxane under a nitrogen atmosphere, and the mixture was stirred and refluxed. Then, potassium acetate (5.5 g, 56 mmol) was added and stirred sufficiently, followed by adding bis(dibenzylideneacetone)palladium(0) (0.6 g, 1.1 mmol) and tricyclohexylphosphine (0.6 g, 2.2 mmol). After 7 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.3 g of subDB-2 (yield 67%, MS: [M+H]+=494).

subDB-2 (15 g, 30.4 mmol) and Trz2 (8.5 g, 31.9 mmol) were added to 300 ml of THF under a nitrogen atmosphere, and the mixture was stirred and refluxed. Then, potassium carbonate (12.6 g, 91.2 mmol) was dissolved in 38 ml of water, and then added thereto. Thereafter, it was stirred sufficiently, followed by adding bis(tri-tert-butylphosphine)palladium(0) (0.2 g, 0.3 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.6 g of Compound 1-16 (yield 64%, MS: [M+H]+=599).

### Synthesis Example 1-17

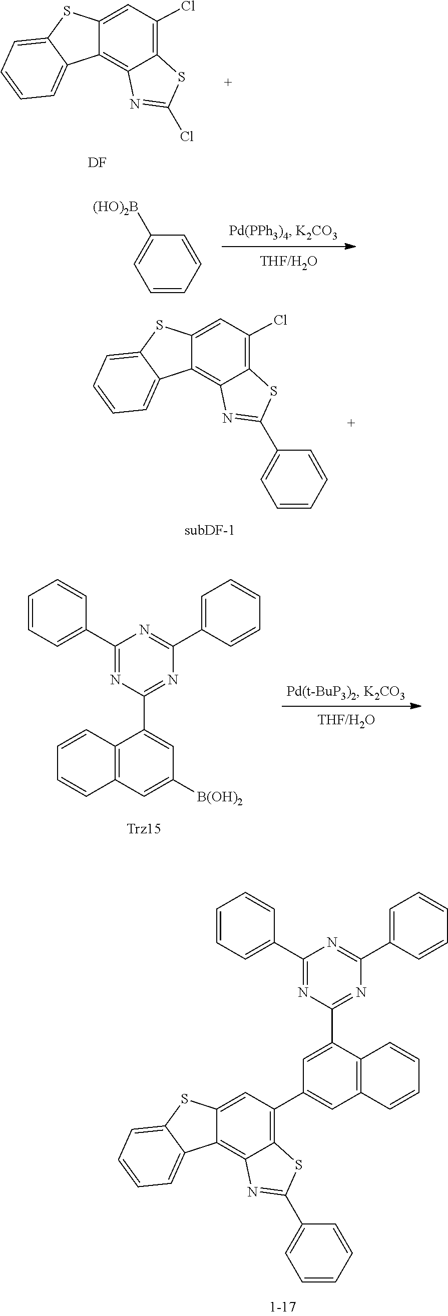

Chemical Formula DF (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9 g of subDF-1 (yield 53%, MS: [M+H]+=352).

subDF-1 (15 g, 42.6 mmol) and Trz15 (18 g, 44.8 mmol) were added to 300 ml of THF under a nitrogen atmosphere, and the mixture was stirred and refluxed. Then, potassium carbonate (17.7 g, 127.9 mmol) was dissolved in 53 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.5 g of Compound 1-17 (yield 68%, MS: [M+H]+=675).

### Synthesis Example 2-1

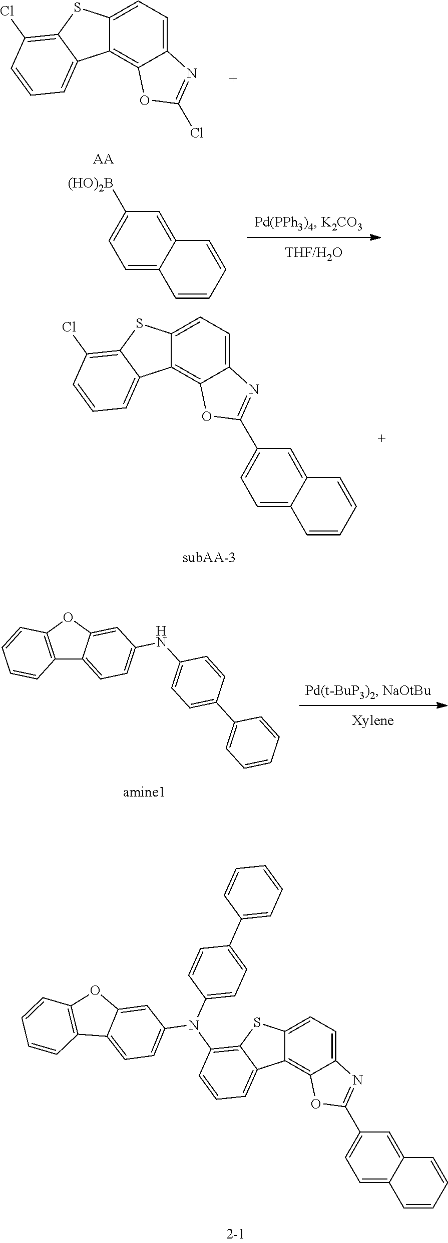

Chemical Formula AA (15 g, 51 mmol) and naphthalen-2-ylboronic acid (9.2 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.2 g of subAA-3 (yield 57%, MS: [M+H]+=386).

subAA-3 (10 g, 25.9 mmol), amine1 (8.7 g, 25.9 mmol), and sodium tert-butoxide (8.3 g, 38.9 mmol) were added to 200 ml of Xylene under a nitrogen atmosphere, and the mixture was stirred and refluxed. Then, bis(tri-tert-butylphosphine)palladium(0) (0.1 g, 0.3 mmol) was added thereto. After 2 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9.6 g of Compound 2-1 (yield 54%, MS: [M+H]+=685).

### Synthesis Example 2-2

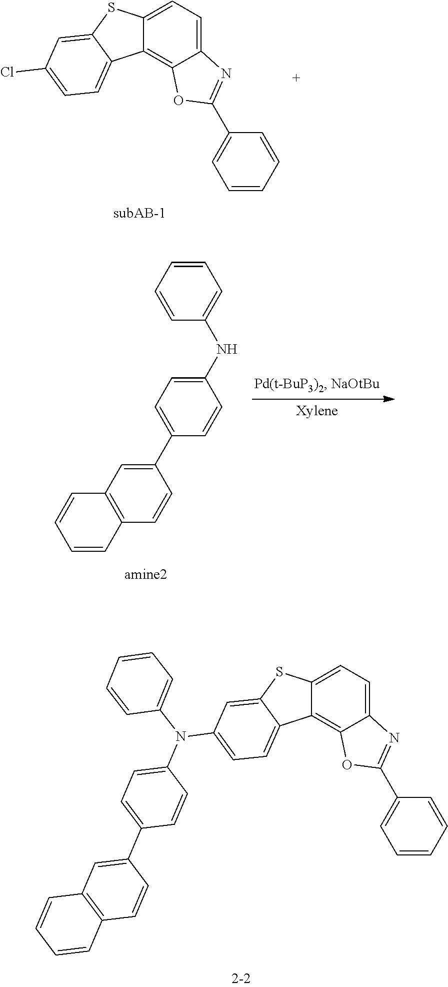 [SMILES: Clc1ccc2c(c1)sc1ccc3nc(-c4ccccc4)oc3c12 ; c1ccc(Nc2ccc(-c3ccc4ccccc4c3)cc2)cc1 ; c1ccc(-c2nc3ccc4sc5cc(N(c6ccccc6)c6ccc(-c7ccc8ccccc8c7)cc6)ccc5c4c3o2)cc1]

subAB-1 (10 g, 29.8 mmol), amine2 (8.8 g, 29.8 mmol), and sodium tert-butoxide (9.5 g, 44.7 mmol) were added to 200 ml of Xylene under a nitrogen atmosphere, and the mixture was stirred and refluxed. Then, bis(tri-tert-butylphosphine)palladium(0) (0.2 g, 0.3 mmol) was added thereto. After 2 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9.6 g of Compound 2-2 (yield 54%, MS: [M+H]+=595).

### Synthesis Example 2-3

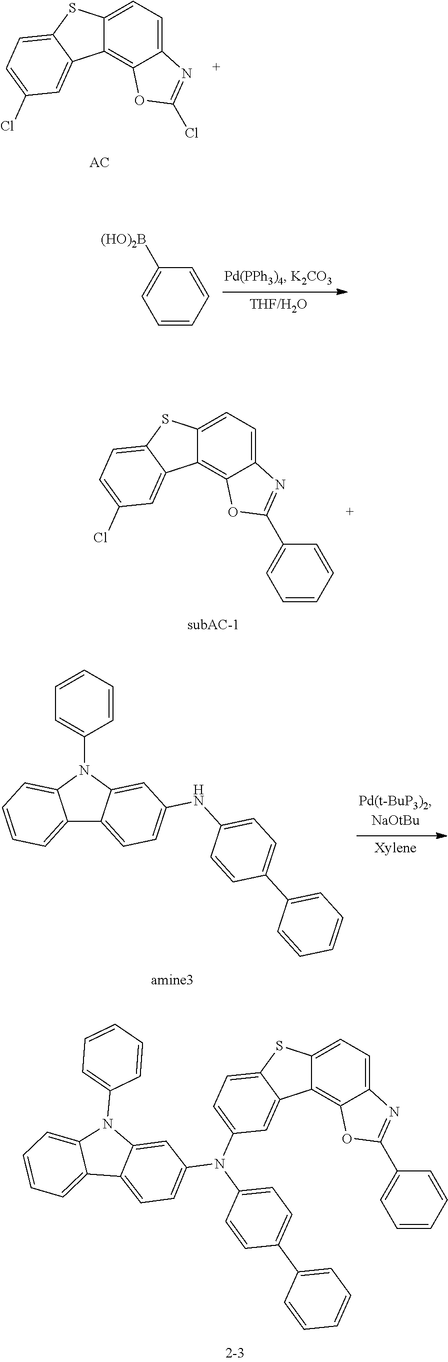 [SMILES: Clc1ccc2sc3ccc4nc(Cl)oc4c3c2c1 ; OB(O)c1ccccc1 ; Clc1ccc2sc3ccc4nc(-c5ccccc5)oc4c3c2c1 ; c1ccc(-c2ccc(Nc3ccc4c5ccccc5n(-c5ccccc5)c4c3)cc2)cc1 ; c1ccc(-c2ccc(N(c3ccc4sc5ccc6nc(-c7ccccc7)oc6c5c4c3)c3ccc4c5ccccc5n(-c5ccccc5)c4c3)cc2)cc1]

Chemical Formula AC (15 g, 51 mmol) and phenylboronic acid (6.5 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.4 g of subAC-1 (yield 55%, MS: [M+H]+=336).

subAC-1 (10 g, 29.8 mmol), amine3 (12.2 g, 29.8 mmol), and sodium tert-butoxide (9.5 g, 44.7 mmol) were added to 200 ml of Xylene under a nitrogen atmosphere, and the mixture was stirred and refluxed. Then, bis(tri-tert-butylphosphine)palladium(0) (0.2 g, 0.3 mmol) was added thereto. After 3 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.2 g of Compound 2-3 (yield 53%, MS: [M+H]+=710).

### Synthesis Example 2-4

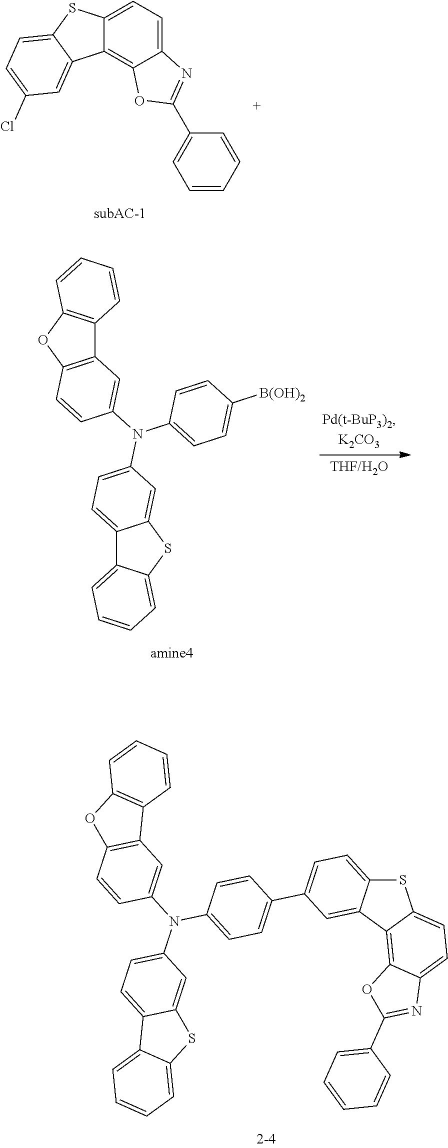 [SMILES: Clc1ccc2sc3ccc4nc(-c5ccccc5)oc4c3c2c1 ; OB(O)c1ccc(N(c2ccc3c(c2)sc2ccccc23)c2ccc3oc4ccccc4c3c2)cc1 ; c1ccc(-c2nc3ccc4sc5ccc(-c6ccc(N(c7ccc8c(c7)sc7ccccc78)c7ccc8oc9ccccc9c8c7)cc6)cc5c4c3o2)cc1]

subAC-1 (15 g, 44.7 mmol) and amine4 (22.8 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16.5 g of Compound 2-4 (yield 50%, MS: [M+H]+=741).

### Synthesis Example 2-5

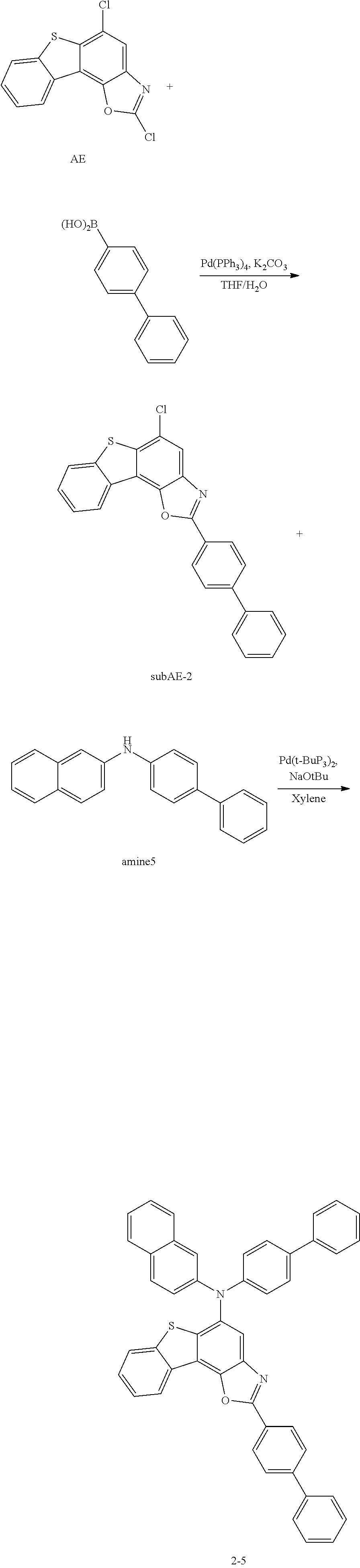 [SMILES: Clc1nc2cc(Cl)c3sc4ccccc4c3c2o1 ; OB(O)c1ccc(-c2ccccc2)cc1 ; Clc1cc2nc(-c3ccc(-c4ccccc4)cc3)oc2c2c1sc1ccccc12 ; c1ccc(-c2ccc(Nc3ccc4ccccc4c3)cc2)cc1 ; c1ccc(-c2ccc(-c3nc4cc(N(c5ccc(-c6ccccc6)cc5)c5ccc6ccccc6c5)c5sc6ccccc6c5c4o3)cc2)cc1]

Chemical Formula AE (15 g, 51 mmol) and [1,1′-biphenyl]-4-ylboronic acid (10.6 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.1 g of subAE-2 (yield 53%, MS: [M+H]+=412).

subAE-2 (10 g, 24.3 mmol), amine5 (7.2 g, 24.3 mmol), and sodium tert-butoxide (7.7 g, 36.4 mmol) were added to 200 ml of Xylene under a nitrogen atmosphere, and the mixture was stirred and refluxed. Then, bis(tri-tert-butylphosphine)palladium(0) (0.1 g, 0.2 mmol) was added thereto. After 2 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.9 g of Compound 2-5 (yield 55%, MS: [M+H]+=671).

### Synthesis Example 2-6

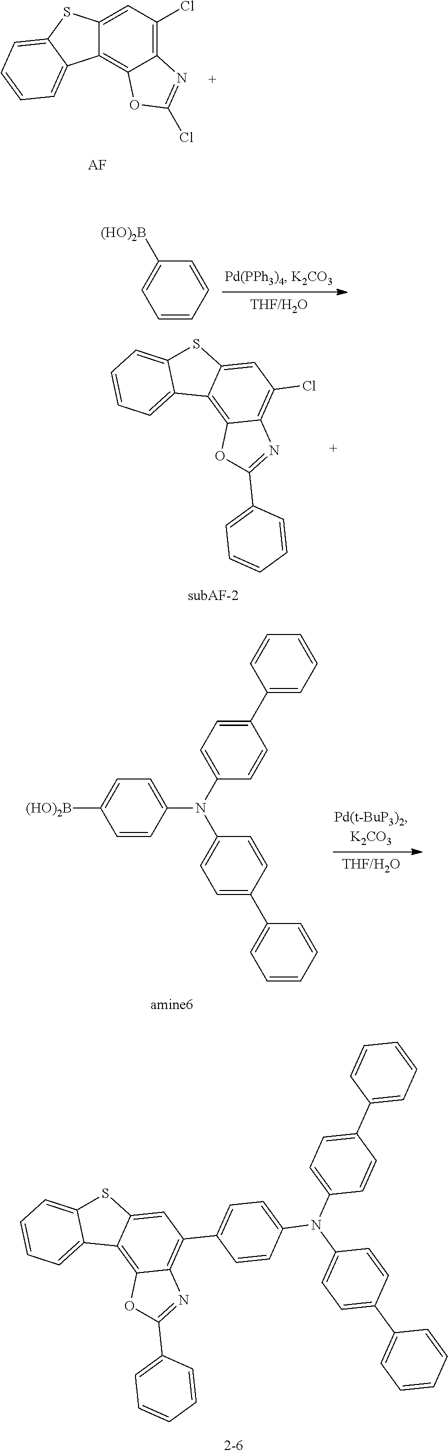

Chemical Formula AF (15 g, 51 mmol) and phenylboronic acid (6.5 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.3 g of subAF-2 (yield 66%, MS: [M+H]+=336).

subAF-2 (15 g, 44.7 mmol) and amine6 (20.7 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16.8 g of Compound 2-6 (yield 54%, MS: [M+H]+=697).

### Synthesis Example 2-7

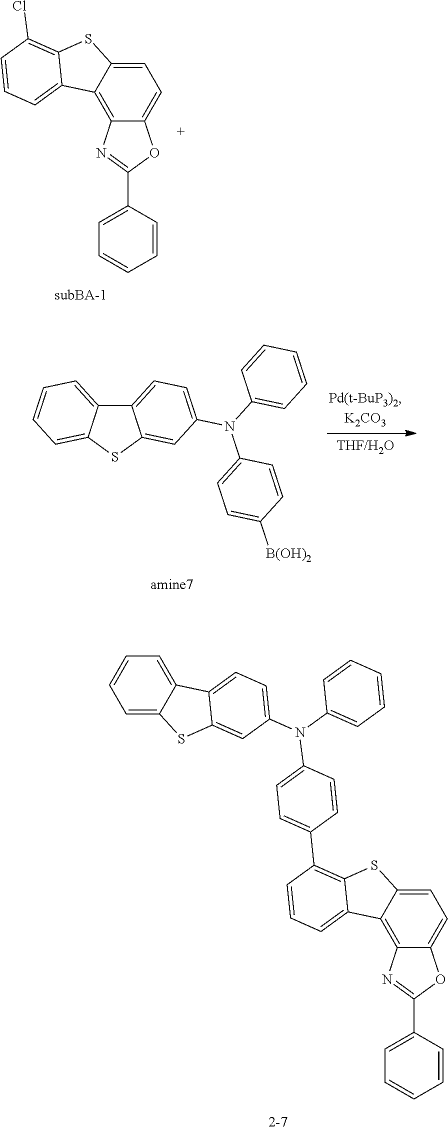 [SMILES: Clc1cccc2c1sc1ccc3oc(-c4ccccc4)nc3c12 ; OB(O)c1ccc(N(c2ccccc2)c2ccc3c(c2)sc2ccccc23)cc1 ; c1ccc(-c2nc3c(ccc4sc5c(-c6ccc(N(c7ccccc7)c7ccc8c(c7)sc7ccccc78)cc6)cccc5c43)o2)cc1]

subBA-1 (15 g, 44.7 mmol) and amine7 (18.5 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.1 g of Compound 2-7 (yield 59%, MS: [M+H]+=651).

### Synthesis Example 2-8

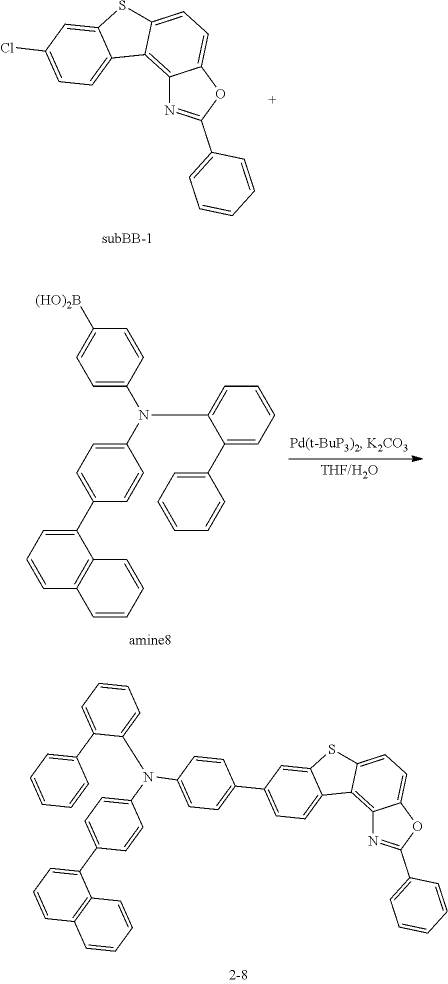 [SMILES: Clc1ccc2c(c1)sc1ccc3oc(-c4ccccc4)nc3c12 ; OB(O)c1ccc(N(c2ccc(-c3cccc4ccccc34)cc2)c2ccccc2-c2ccccc2)cc1 ; c1ccc(-c2nc3c(ccc4sc5cc(-c6ccc(N(c7ccc(-c8cccc9ccccc89)cc7)c7ccccc7-c7ccccc7)cc6)ccc5c43)o2)cc1]

subBB-1 (15 g, 44.7 mmol) and amine8 (23 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22 g of Compound 2-8 (yield 66%, MS: [M+H]+=747).

### Synthesis Example 2-9

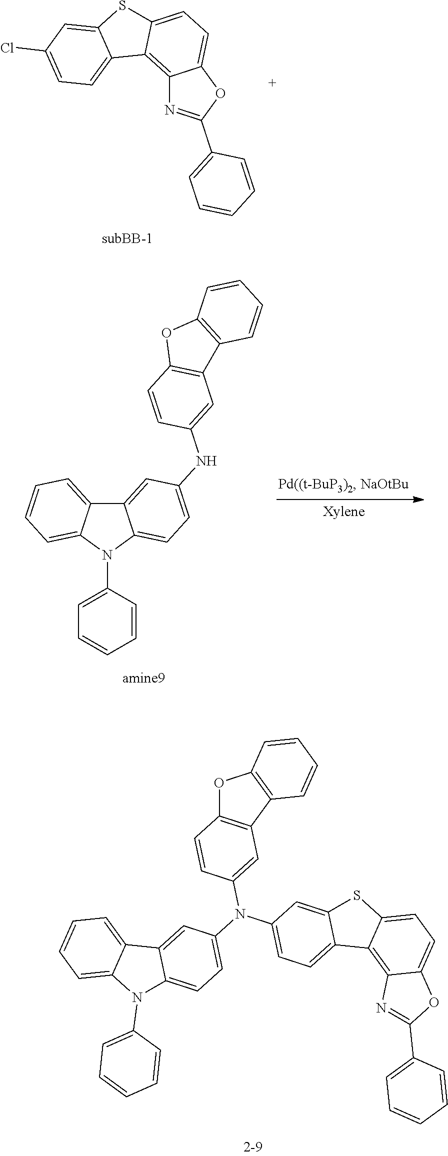 [SMILES: Clc1ccc2c(c1)sc1ccc3oc(-c4ccccc4)nc3c12 ; c1ccc(-n2c3ccccc3c3cc(Nc4ccc5oc6ccccc6c5c4)ccc32)cc1 ; c1ccc(-c2nc3c(ccc4sc5cc(N(c6ccc7oc8ccccc8c7c6)c6ccc7c(c6)c6ccccc6n7-c6ccccc6)ccc5c43)o2)cc1]

subBB-1 (10 g, 29.8 mmol), amine9 (12.6 g, 29.8 mmol), and sodium tert-butoxide (9.5 g, 44.7 mmol) were added to 200 ml of Xylene under a nitrogen atmosphere, and the mixture was stirred and refluxed. Then, bis(tri-tert-butylphosphine)palladium(0) (0.2 g, 0.3 mmol) was added thereto. After 2 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.4 g of Compound 2-9 (yield 53%, MS: [M+H]+=724).

### Synthesis Example 2-10

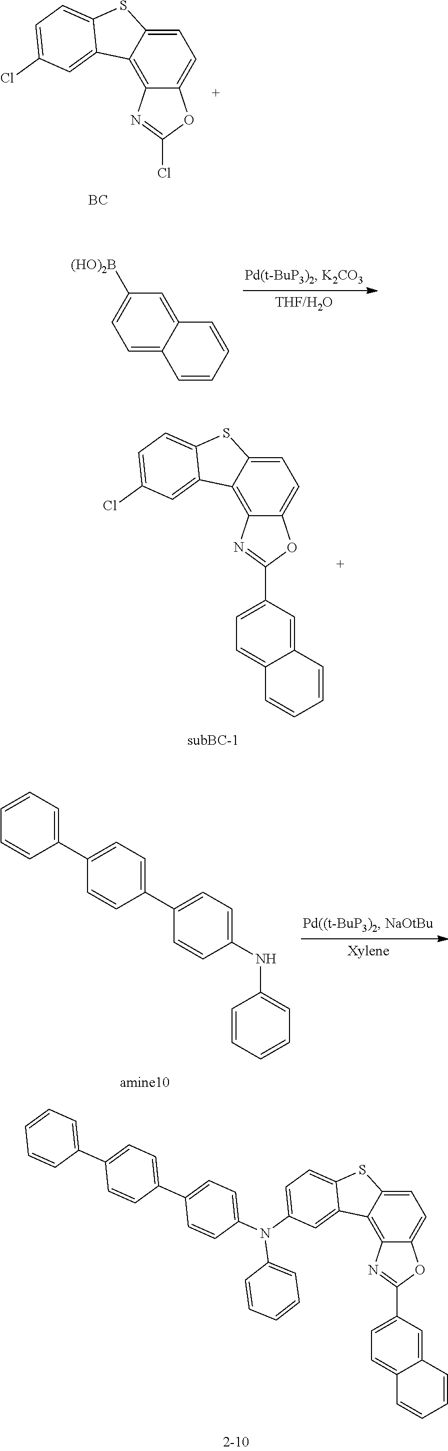 [SMILES: Clc1ccc2sc3ccc4oc(Cl)nc4c3c2c1 ; "BC" ; OB(O)c1ccc2ccccc2c1 ; Clc1ccc2sc3ccc4oc(-c5ccc6ccccc6c5)nc4c3c2c1 ; c1ccc(Nc2ccc(-c3ccc(-c4ccccc4)cc3)cc2)cc1 ; c1ccc(-c2ccc(-c3ccc(N(c4ccccc4)c4ccc5sc6ccc7oc(-c8ccc9ccccc9c8)nc7c6c5c4)cc3)cc2)cc1]

Chemical Formula BC (15 g, 51 mmol) and naphthalen-2-ylboronic acid (9.2 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.7 g of subBC-1 (yield 70%, MS: [M+H]+=386).

subBC-1 (10 g, 25.9 mmol), amine10 (8.3 g, 25.9 mmol), and sodium tert-butoxide (8.3 g, 38.9 mmol) were added to 200 ml of Xylene under a nitrogen atmosphere, and the mixture was stirred and refluxed. Then, bis(tri-tert-butylphosphine)palladium(0) (0.1 g, 0.3 mmol) was added thereto. After 3 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9 g of Compound 2-10 (yield 52%, MS: [M+H]+=671).

### Synthesis Example 2-11

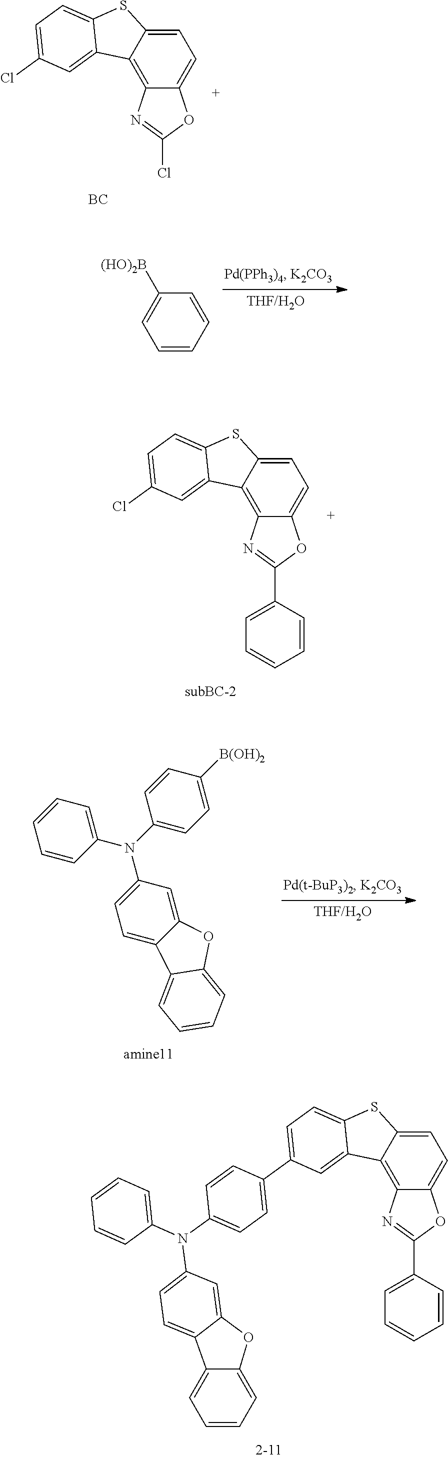 [SMILES: Clc1ccc2sc3ccc4oc(Cl)nc4c3c2c1 ; "BC" ; OB(O)c1ccccc1 ; Clc1ccc2sc3ccc4oc(-c5ccccc5)nc4c3c2c1 ; OB(O)c1ccc(N(c2ccccc2)c2ccc3c(c2)oc2ccccc23)cc1 ; c1ccc(-c2nc3c(ccc4sc5ccc(-c6ccc(N(c7ccccc7)c7ccc8c(c7)oc7ccccc78)cc6)cc5c43)o2)cc1]

Chemical Formula BC (15 g, 51 mmol) and phenylboronic acid (6.5 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.3 g of subBC-2 (yield 60%, MS: [M+H]+=336).

subBC-2 (15 g, 44.7 mmol) and amine11 (17.8 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.4 g of Compound 2-11 (yield 51%, MS: [M+H]+=635).

### Synthesis Example 2-12

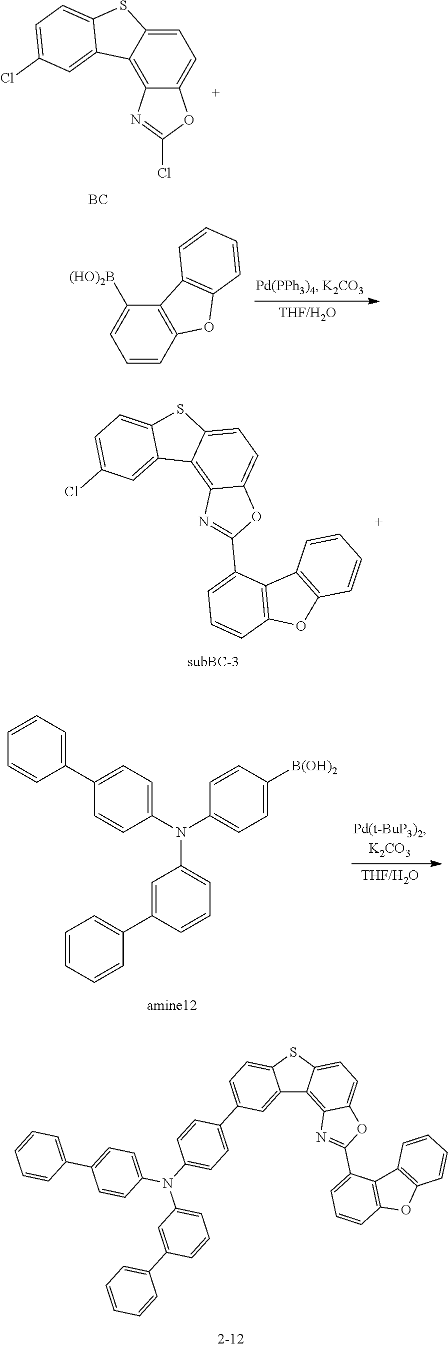 [SMILES: Clc1ccc2sc3ccc4oc(Cl)nc4c3c2c1 ; "BC" ; OB(O)c1cccc2oc3ccccc3c12 ; Clc1ccc2sc3ccc4oc(-c5cccc6oc7ccccc7c56)nc4c3c2c1 ; OB(O)c1ccc(N(c2ccc(-c3ccccc3)cc2)c2cccc(-c3ccccc3)c2)cc1 ; c1ccc(-c2ccc(N(c3ccc(-c4ccc5sc6ccc7oc(-c8cccc9oc%10ccccc%10c89)nc7c6c5c4)cc3)c3cccc(-c4ccccc4)c3)cc2)cc1]

Chemical Formula BC (15 g, 51 mmol) and dibenzo[b,d]furan-1-ylboronic acid (11.4 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.3 g of subBC-3 (yield 66%, MS: [M+H]+=426).

subBC-3 (15 g, 35.2 mmol) and amine12 (16.3 g, 37 mmol) were added to 300 ml of THF under a nitrogen atmosphere, and the mixture was stirred and refluxed. Then, potassium carbonate (14.6 g, 105.7 mmol) was dissolved in 44 ml of water, and then added thereto. Thereafter, it was stirred sufficiently, followed by adding bis(tri-tert-butylphosphine)palladium(0) (0.2 g, 0.4 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.1 g of Compound 2-12 (yield 69%, MS: [M+H]+=787).

### Synthesis Example 2-13

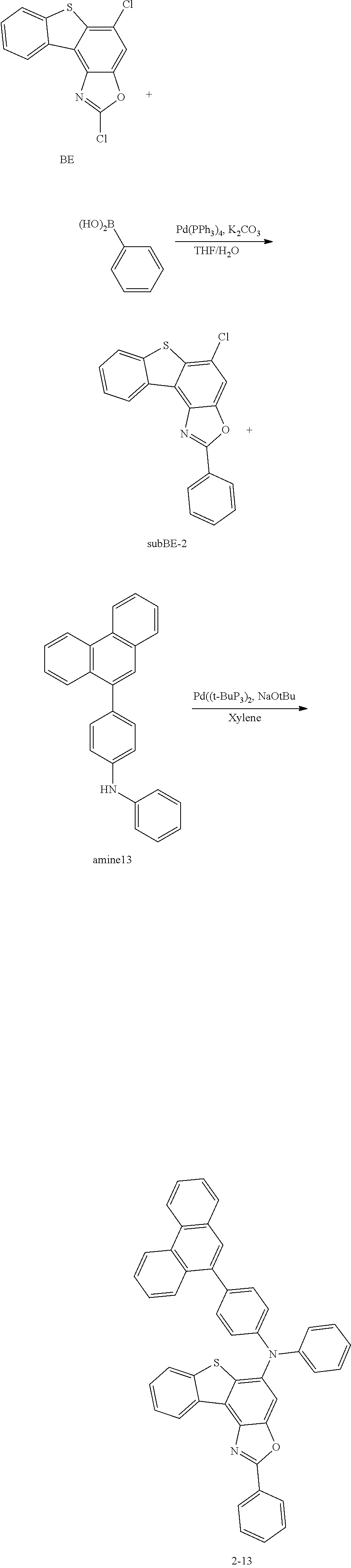 [SMILES: Clc1nc2c(cc(Cl)c3sc4ccccc4c32)o1 ; "BE" ; OB(O)c1ccccc1 ; Clc1cc2oc(-c3ccccc3)nc2c2c1sc1ccccc12 ; c1ccc(Nc2ccc(-c3cc4ccccc4c4ccccc34)cc2)cc1 ; c1ccc(-c2nc3c(cc(N(c4ccccc4)c4ccc(-c5cc6ccccc6c6ccccc56)cc4)c4sc5ccccc5c43)o2)cc1]

Chemical Formula BE (15 g, 51 mmol) and phenylboronic acid (6.5 g, 53.5 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 8.5 g of subBE-2 (yield 50%, MS: [M+H]+=336).

subBE-2 (10 g, 29.8 mmol), amine13 (10.3 g, 29.8 mmol), and sodium tert-butoxide (9.5 g, 44.7 mmol) were added to 200 ml of Xylene under a nitrogen atmosphere, and the mixture was stirred and refluxed. Then, bis(tri-tert-butylphosphine)palladium(0) (0.2 g, 0.3 mmol) was added thereto. After 3 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.5 g of Compound 2-13 (yield 60%, MS: [M+H]+=645).

### Synthesis Example 2-14

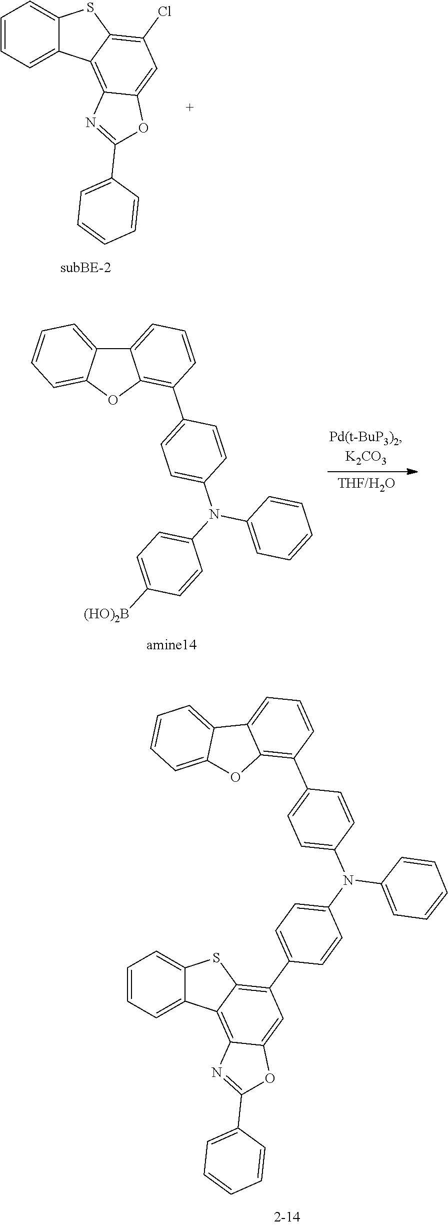 [SMILES: Clc1cc2oc(-c3ccccc3)nc2c2c1sc1ccccc12 ; OB(O)c1ccc(N(c2ccccc2)c2ccc(-c3cccc4c3oc3ccccc34)cc2)cc1 ; c1ccc(-c2nc3c(cc(-c4ccc(N(c5ccccc5)c5ccc(-c6cccc7c6oc6ccccc67)cc5)cc4)c4sc5ccccc5c43)o2)cc1]

subBE-2 (15 g, 44.7 mmol) and amine14 (21.4 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.3 g of Compound 2-14 (yield 64%, MS: [M+H]+=711).

### Synthesis Example 2-15

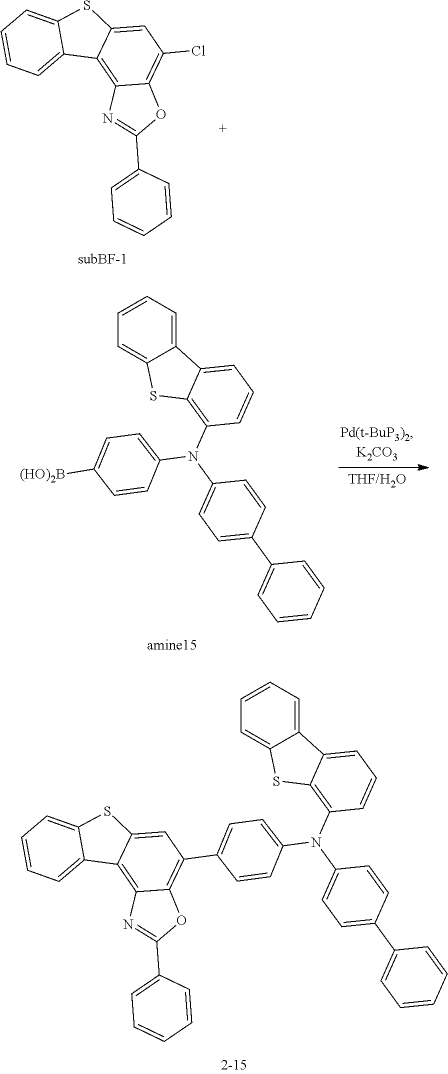 [SMILES: Clc1cc2sc3ccccc3c2c2nc(-c3ccccc3)oc12 ; OB(O)c1ccc(N(c2ccc(-c3ccccc3)cc2)c2cccc3c2sc2ccccc23)cc1 ; c1ccc(-c2ccc(N(c3ccc(-c4cc5sc6ccccc6c5c5nc(-c6ccccc6)oc45)cc3)c3cccc4c3sc3ccccc34)cc2)cc1]

subBF-1 (15 g, 44.7 mmol) and amine15 (22.1 g, 46.9 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.8 g of Compound 2-15 (yield 55%, MS: [M+H]+=727).

### Synthesis Example 2-16

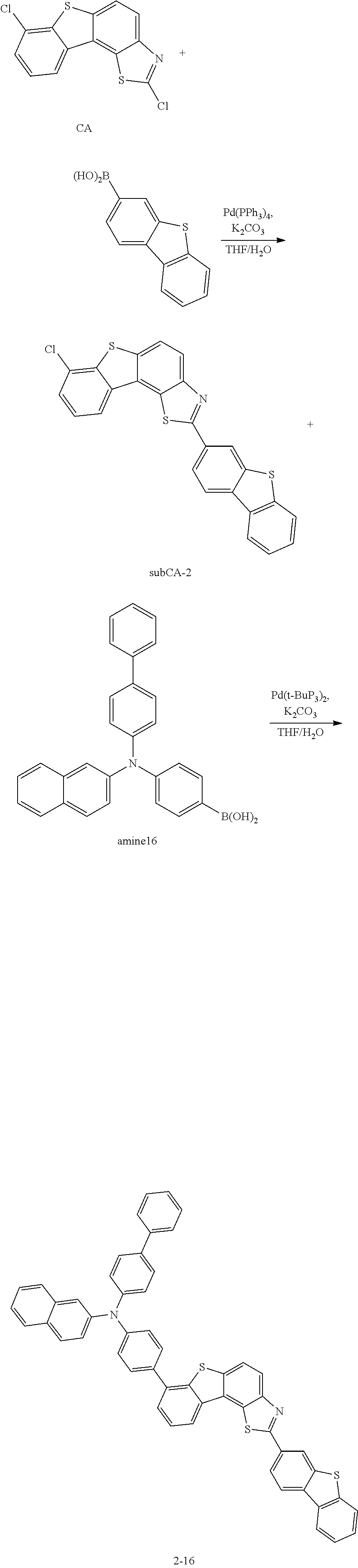 [SMILES: Clc1nc2ccc3sc4c(Cl)cccc4c3c2s1 ; OB(O)c1ccc2c(c1)sc1ccccc12 ; Clc1cccc2c1sc1ccc3nc(-c4ccc5c(c4)sc4ccccc45)sc3c12 ; OB(O)c1ccc(N(c2ccc(-c3ccccc3)cc2)c2ccc3ccccc3c2)cc1 ; c1ccc(-c2ccc(N(c3ccc(-c4cccc5c4sc4ccc6nc(-c7ccc8c(c7)sc7ccccc78)sc6c45)cc3)c3ccc4ccccc4c3)cc2)cc1]

Chemical Formula CA (15 g, 48.4 mmol) and dibenzo[b,d]thiophen-3-ylboronic acid (11.6 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.3 g of subCA-2 (yield 51%, MS: [M+H]+=458).

subCA-2 (15 g, 32.8 mmol) and amine16 (14.3 g, 34.4 mmol) were added to 300 ml of THF under a nitrogen atmosphere, and the mixture was stirred and refluxed. Then, potassium carbonate (13.6 g, 98.3 mmol) was dissolved in 41 ml of water, and then added thereto. Thereafter, it was stirred sufficiently, followed by adding bis(tri-tert-butylphosphine)palladium(0) (0.2 g, 0.3 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.3 g of Compound 2-16 (yield 55%, MS: [M+H]+=793).

### Synthesis Example 2-17

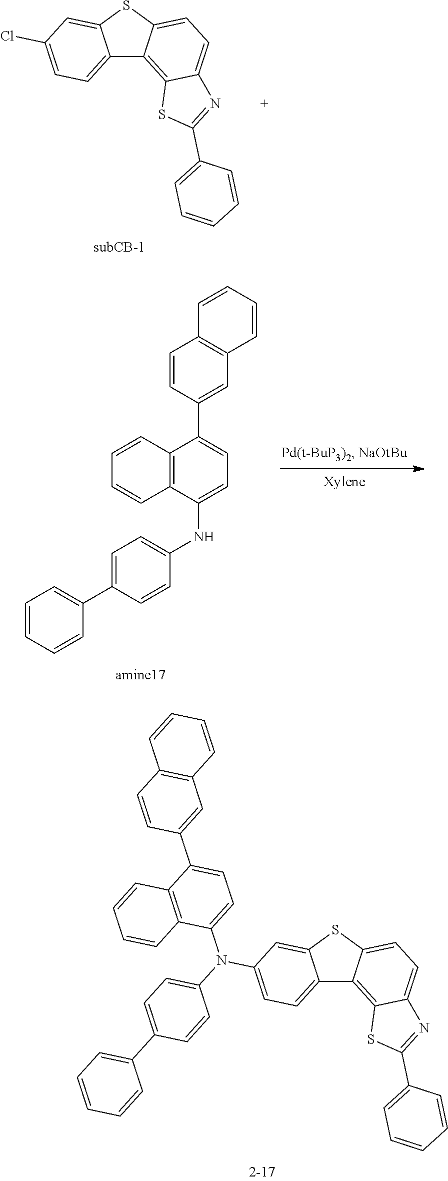 [SMILES: Clc1ccc2c(c1)sc1ccc3nc(-c4ccccc4)sc3c12 ; c1ccc(-c2ccc(Nc3ccc(-c4ccc5ccccc5c4)c4ccccc34)cc2)cc1 ; c1ccc(-c2ccc(N(c3ccc4c(c3)sc3ccc5nc(-c6ccccc6)sc5c34)c3ccc(-c4ccc5ccccc5c4)c4ccccc34)cc2)cc1]

subCB-1 (10 g, 28.4 mmol), amine17 (12 g, 28.4 mmol), and sodium tert-butoxide (9 g, 42.6 mmol) were added to 200 ml of Xylene under a nitrogen atmosphere, and the mixture was stirred and refluxed. Then, bis(tri-tert-butylphosphine)palladium(0) (0.1 g, 0.3 mmol) was added thereto. After 3 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.2 g of Compound 2-17 (yield 63%, MS: [M+H]+=737).

### Synthesis Example 2-18

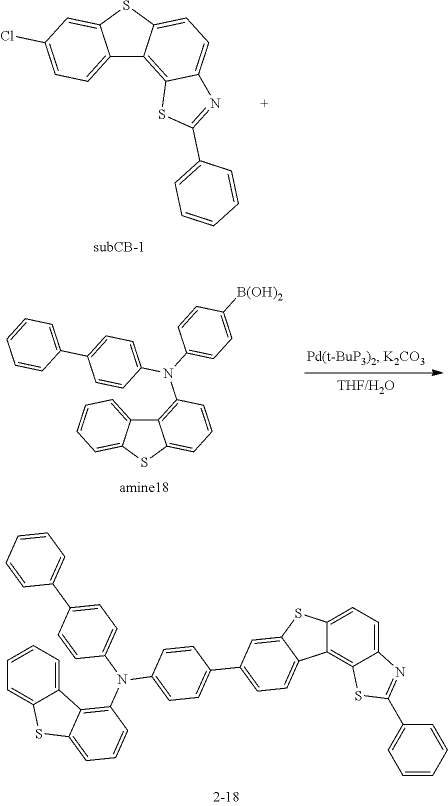 [SMILES: Clc1ccc2c(c1)sc1ccc3nc(-c4ccccc4)sc3c12 ; OB(O)c1ccc(N(c2ccc(-c3ccccc3)cc2)c2cccc3sc4ccccc4c23)cc1 ; c1ccc(-c2ccc(N(c3ccc(-c4ccc5c(c4)sc4ccc6nc(-c7ccccc7)sc6c45)cc3)c3cccc4sc5ccccc5c34)cc2)cc1]

subCB-1 (15 g, 42.6 mmol) and amine18 (21.1 g, 44.8 mmol) were added to 300 ml of THF under a nitrogen atmosphere, and the mixture was stirred and refluxed. Then, potassium carbonate (17.7 g, 127.9 mmol) was dissolved in 53 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18 g of Compound 2-18 (yield 57%, MS: [M+H]+=743).

### Synthesis Example 2-19

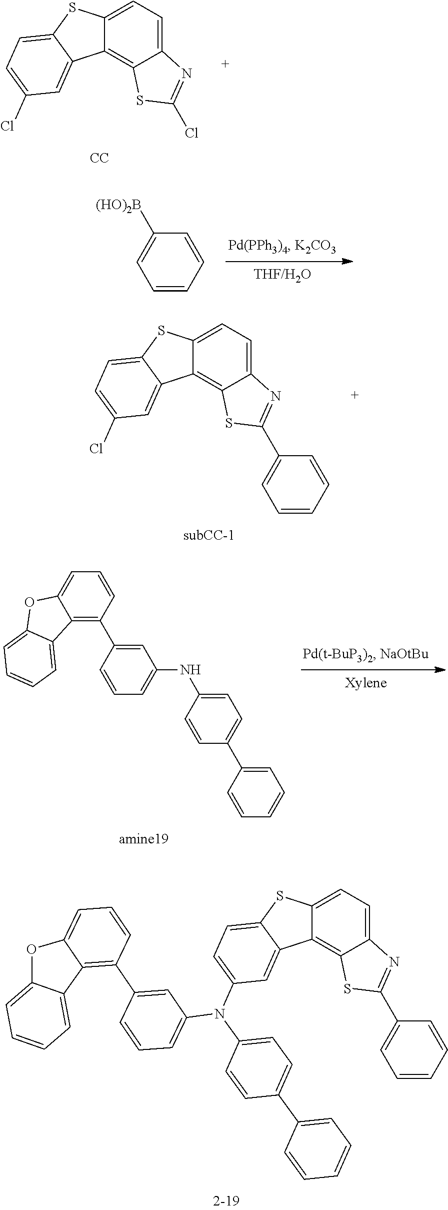

Chemical Formula CC (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 8.7 g of subCC-1 (yield 51%, MS: [M+H]+=352).

subCC-1 (10 g, 28.4 mmol), amine19 (11.7 g, 28.4 mmol), and sodium tert-butoxide (9 g, 42.6 mmol) were added to 200 ml of Xylene under a nitrogen atmosphere, and the mixture was stirred and refluxed. Then, bis(tri-tert-butylphosphine)palladium(0) (0.1 g, 0.3 mmol) was added thereto. After 3 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.4 g of Compound 2-19 (yield 65%, MS: [M+H]+=727).

### Synthesis Example 2-20

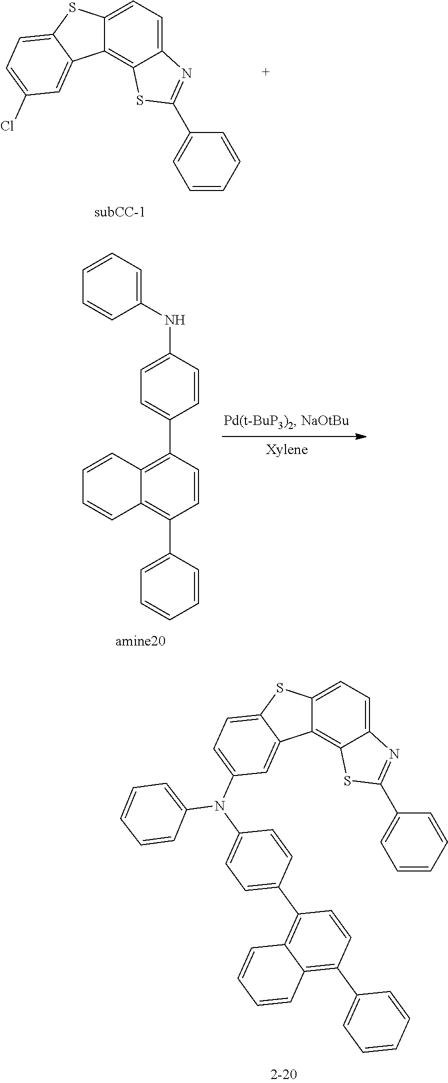

subCC-1 (10 g, 28.4 mmol), amine20 (10.6 g, 28.4 mmol), and sodium tert-butoxide (9 g, 42.6 mmol) were added to 200 ml of Xylene under a nitrogen atmosphere, and the mixture was stirred and refluxed. Then, bis(tri-tert-butylphosphine)palladium(0) (0.1 g, 0.3 mmol) was added thereto. After 2 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.3 g of Compound 2-20 (yield 63%, MS: [M+H]+=687).

### Synthesis Example 2-21

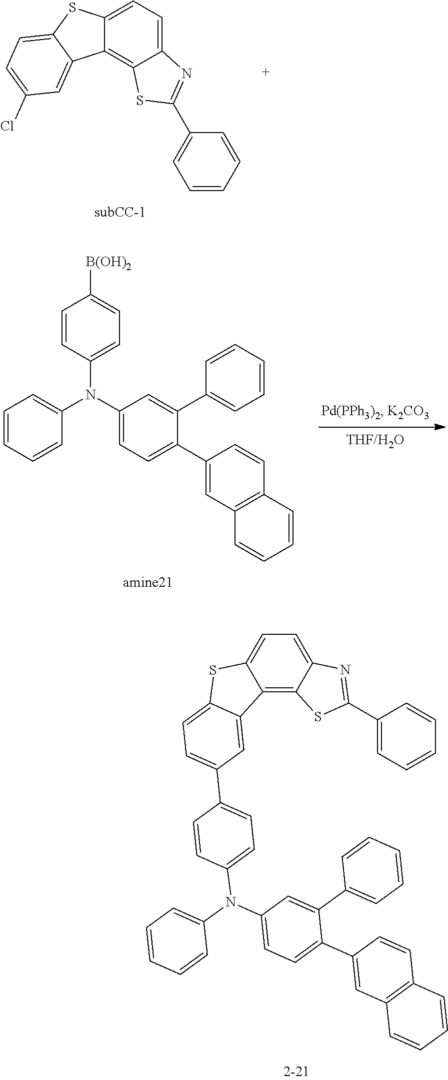 [SMILES: Clc1ccc2sc3ccc4nc(-c5ccccc5)sc4c3c2c1 ; "subCC-1" ; OB(O)c1ccc(N(c2ccccc2)c2ccc(-c3ccc4ccccc4c3)c(-c3ccccc3)c2)cc1 ; c1ccc(-c2nc3ccc4sc5ccc(-c6ccc(N(c7ccccc7)c7ccc(-c8ccc9ccccc9c8)c(-c8ccccc8)c7)cc6)cc5c4c3s2)cc1]

subCC-1 (15 g, 42.6 mmol) and amine21 (22 g, 44.8 mmol) were added to 300 ml of THF under a nitrogen atmosphere, and the mixture was stirred and refluxed. Then, potassium carbonate (17.7 g, 127.9 mmol) was dissolved in 53 ml of water, and then added thereto. Thereafter, it was stirred sufficiently, followed by adding bis(tri-tert-butylphosphine)palladium(0) (0.2 g, 0.4 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.1 g of Compound 2-21 (yield 65%, MS: [M+H]+=763).

### Synthesis Example 2-22

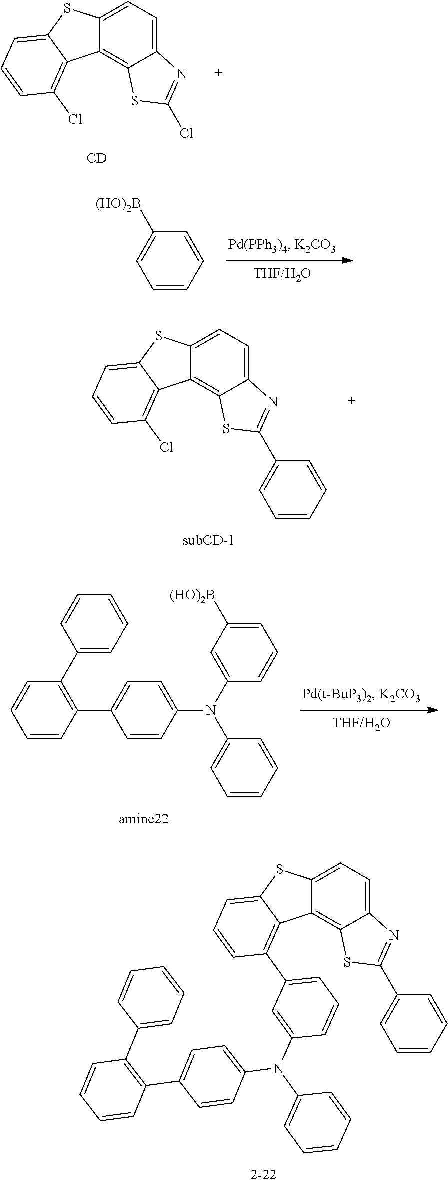

Chemical Formula CD (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 8.7 g of subCD-1 (yield 51%, MS: [M+H]+=352).

subCD-1 (15 g, 42.6 mmol) and amine22 (19.8 g, 44.8 mmol) were added to 300 ml of THF under a nitrogen atmosphere, and the mixture was stirred and refluxed. Then, potassium carbonate (17.7 g, 127.9 mmol) was dissolved in 53 ml of water, and then added thereto. Thereafter, it was stirred sufficiently, followed by adding bis(tri-tert-butylphosphine)palladium(0) (0.2 g, 0.4 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.3 g of Compound 2-22 (yield 57%, MS: [M+H]+=713).

### Synthesis Example 2-23

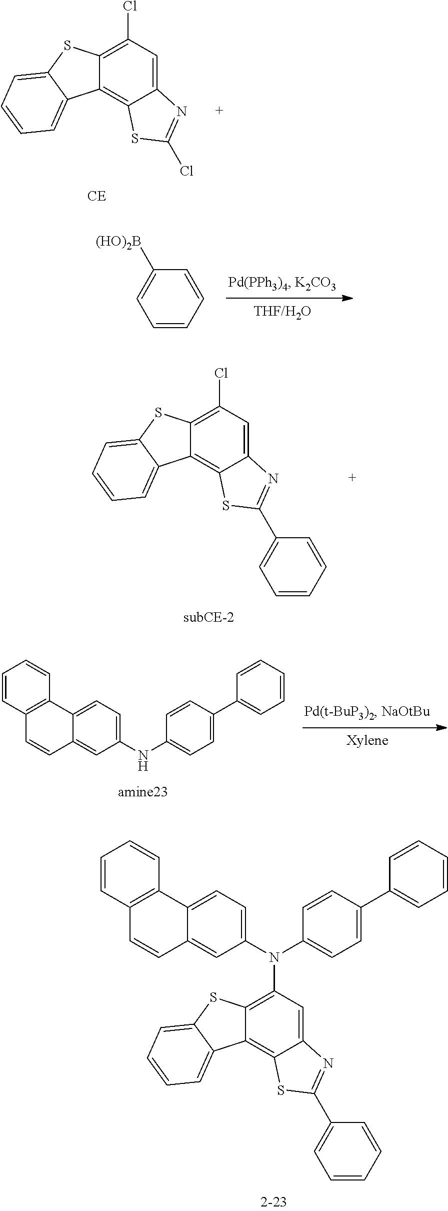

Chemical Formula CE (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.2 g of subCE-2 (yield 66%, MS: [M+H]+=352).

subCE-2 (10 g, 28.4 mmol), amine23 (9.8 g, 28.4 mmol), and sodium tert-butoxide (9 g, 42.6 mmol) were added to 200 ml of Xylene under a nitrogen atmosphere, and the mixture was stirred and refluxed. Then, bis(tri-tert-butylphosphine)palladium(0) (0.1 g, 0.3 mmol) was added thereto. After 2 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.1 g of Compound 2-23 (yield 59%, MS: [M+H]+=661).

### Synthesis Example 2-24

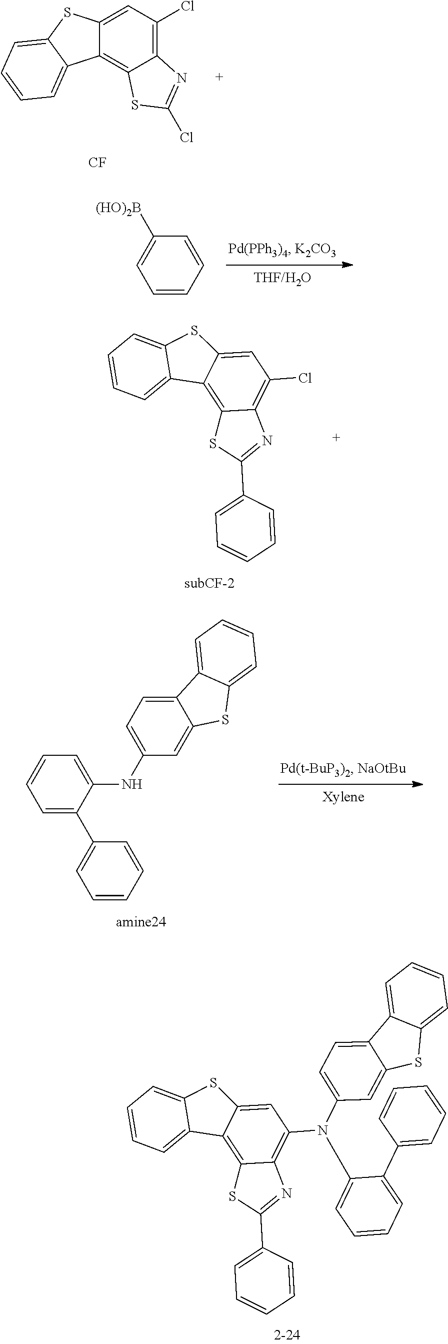

Chemical Formula CF (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.8 g of subCF-2 (yield 58%, MS: [M+H]+=352).

subCF-2 (10 g, 28.4 mmol), amine24 (10 g, 28.4 mmol), and sodium tert-butoxide (9 g, 42.6 mmol) were added to 200 ml of Xylene under a nitrogen atmosphere, and the mixture was stirred and refluxed. Then, bis(tri-tert-butylphosphine)palladium(0) (0.1 g, 0.3 mmol) was added thereto. After 2 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.2 g of Compound 2-24 (yield 54%, MS: [M+H]+=667).

### Synthesis Example 2-25

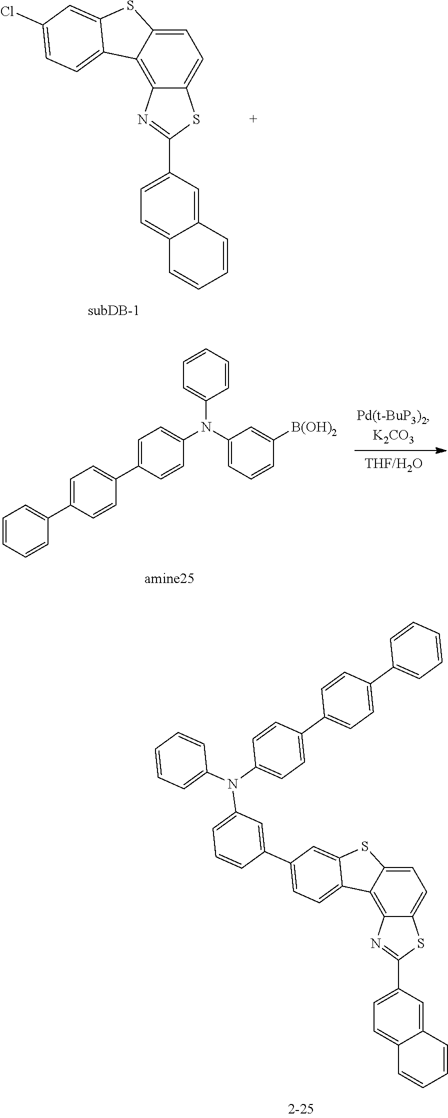 [SMILES: Clc1ccc2c(c1)sc1ccc3sc(-c4ccc5ccccc5c4)nc3c12 ; OB(O)c1cccc(N(c2ccccc2)c2ccc(-c3ccc(-c4ccccc4)cc3)cc2)c1 ; c1ccc(-c2ccc(-c3ccc(N(c4ccccc4)c4cccc(-c5ccc6c(c5)sc5ccc7sc(-c8ccc9ccccc9c8)nc7c56)c4)cc3)cc2)cc1]

subDB-1 (15 g, 37.3 mmol) and amine25 (17.3 g, 39.2 mmol) were added to 300 ml of THF under a nitrogen atmosphere, and the mixture was stirred and refluxed. Then, potassium carbonate (15.5 g, 112 mmol) was dissolved in 46 ml of water, and then added thereto. Thereafter, it was stirred sufficiently, followed by adding bis(tri-tert-butylphosphine)palladium(0) (0.2 g, 0.4 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.8 g of Compound 2-25 (yield 52%, MS: [M+H]+=763).

### Synthesis Example 2-26

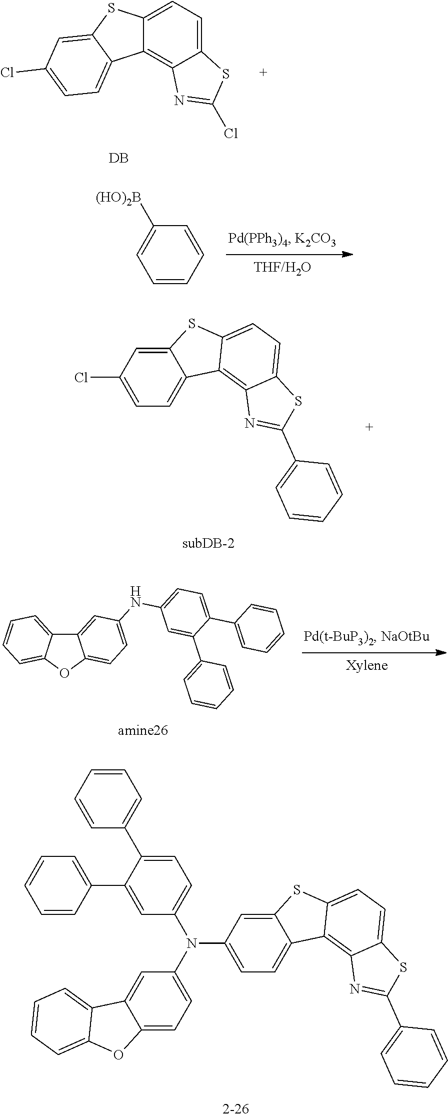

Chemical Formula DC (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.2 g of subDB-2 (yield 66%, MS: [M+H]+=352).

subDB-2 (10 g, 29.8 mmol), amine26 (12.3 g, 29.8 mmol), and sodium tert-butoxide (9.5 g, 44.7 mmol) were added to 200 ml of Xylene under a nitrogen atmosphere, and the mixture was stirred and refluxed. Then, bis(tri-tert-butylphosphine)palladium(0) (0.2 g, 0.3 mmol) was added thereto. After 2 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.4 g of Compound 2-26 (yield 54%, MS: [M+H]+=711).

### Synthesis Example 2-27

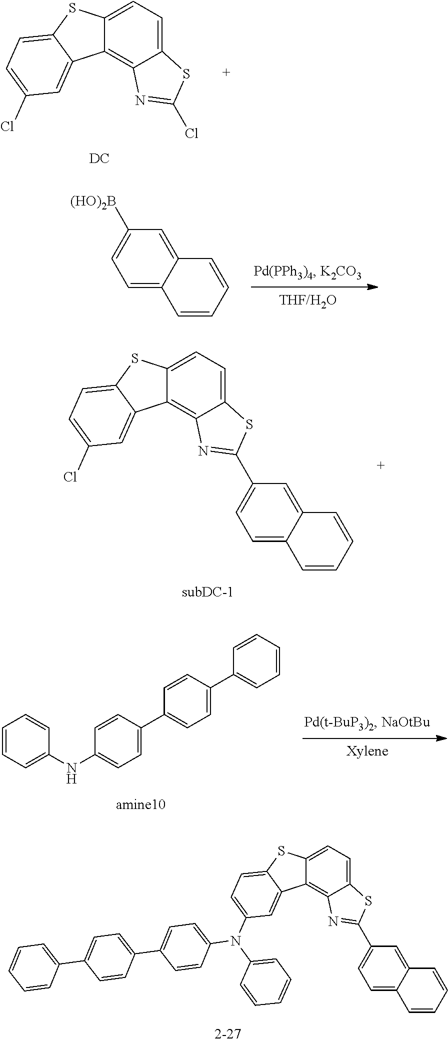

Chemical Formula DC (15 g, 48.4 mmol) and naphthalen-2-ylboronic acid (8.7 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.1 g of subDC-1 (yield 57%, MS: [M+H]+=402).

subDC-1 (10 g, 24.9 mmol), amine10 (8 g, 24.9 mmol), and sodium tert-butoxide (7.9 g, 37.3 mmol) were added to 200 ml of Xylene under a nitrogen atmosphere, and the mixture was stirred and refluxed. Then, bis(tri-tert-butylphosphine)palladium(0) (0.1 g, 0.2 mmol) was added thereto. After 3 hours, the reaction was completed, cooling was performed to room temperature, and the solvent was removed under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.7 g of Compound 2-27 (yield 51%, MS: [M+H]+=687).

### Synthesis Example 2-28

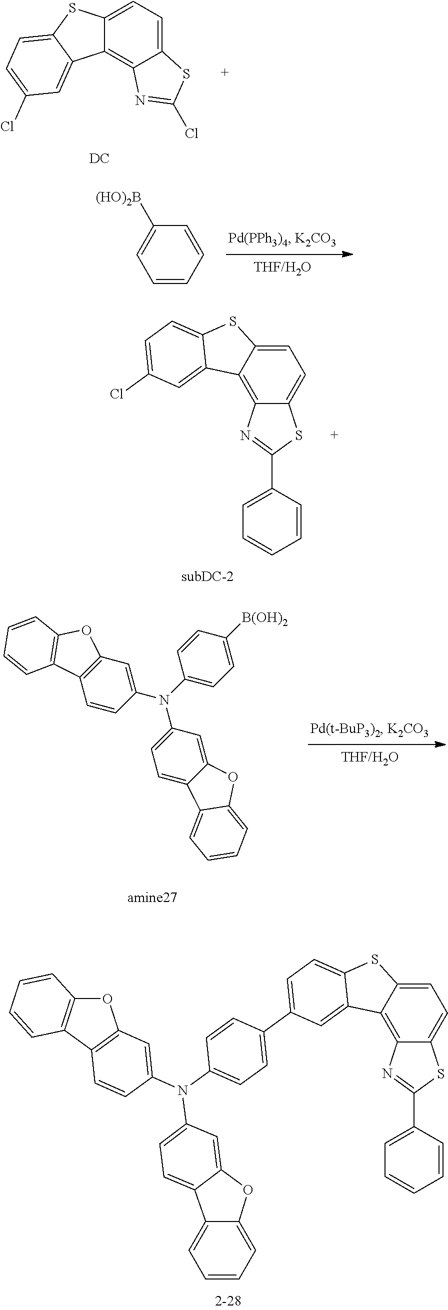

Chemical Formula DC (15 g, 48.4 mmol) and phenylboronic acid (6.2 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9 g of subDC-2 (yield 53%, MS: [M+H]+=352).

subDC-2 (15 g, 42.6 mmol) and amine27 (21 g, 44.8 mmol) were added to 300 ml of THF under a nitrogen atmosphere, and the mixture was stirred and refluxed. Then, potassium carbonate (17.7 g, 127.9 mmol) was dissolved in 53 ml of water, and then added thereto. Thereafter, it was stirred sufficiently, followed by adding bis(tri-tert-butylphosphine)palladium(0) (0.2 g, 0.4 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.1 g of Compound 2-28 (yield 70%, MS: [M+H]+=741).

### Synthesis Example 2-29

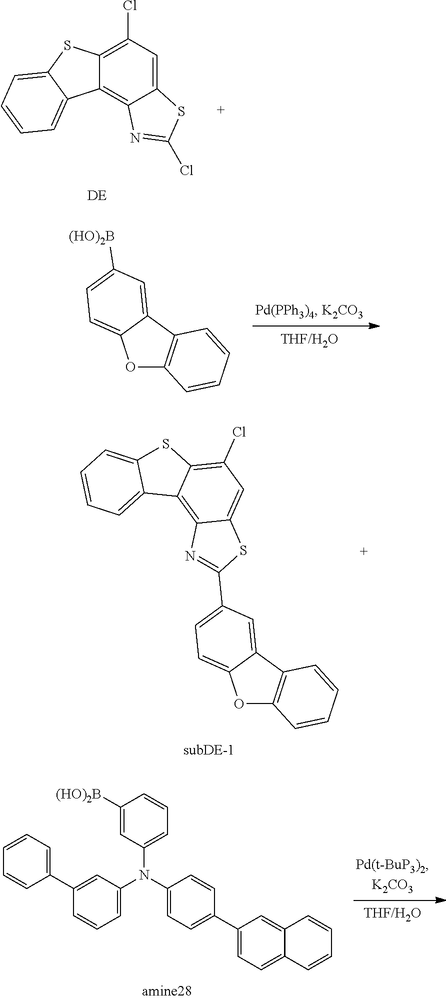

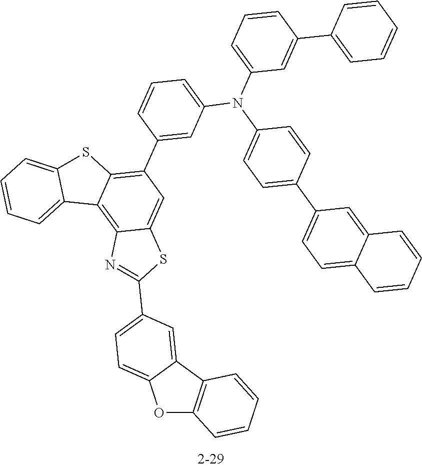 [SMILES: c1ccc(-c2cccc(N(c3ccc(-c4ccc5ccccc5c4)cc3)c3cccc(-c4cc5sc(-c6ccc7oc8ccccc8c7c6)nc5c5c4sc4ccccc45)c3)c2)cc1]

Chemical Formula (15 g, 48.4 mmol) and dibenzo[b,d]furan-2-ylboronic acid (10.8 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.9 g of subDE-1 (yield 56%, MS: [M+H]+=442).

subDE-1 (15 g, 33.9 mmol) and amine28 (17.5 g, 35.6 mmol) were added to 300 ml of THF under a nitrogen atmosphere, and the mixture was stirred and refluxed. Then, potassium carbonate (14.1 g, 101.8 mmol) was dissolved in 42 ml of water, and then added thereto. Thereafter, it was stirred sufficiently, followed by adding bis(tri-tert-butylphosphine)palladium(0) (0.2 g, 0.3 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.1 g of Compound 2-29 (yield 59%, MS: [M+H]+=853).

### Synthesis Example 2-30

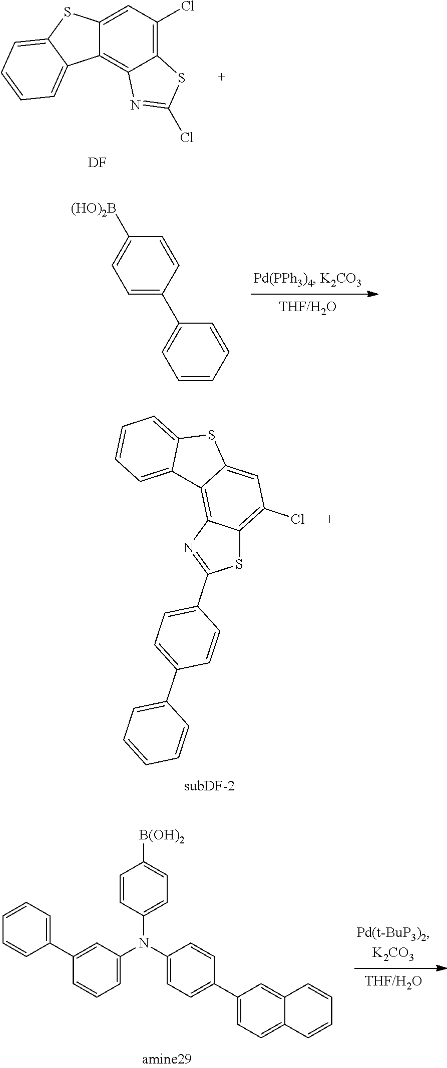

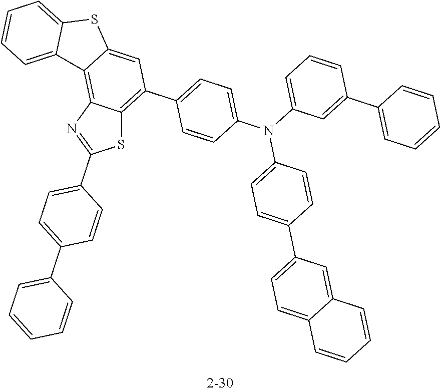 [SMILES: c1ccc(-c2ccc(-c3nc4c(s3)c(-c3ccc(N(c5ccc(-c6ccc7ccccc7c6)cc5)c5cccc(-c6ccccc6)c5)cc3)cc3sc5ccccc5c34)cc2)cc1]

Chemical Formula DF (15 g, 48.4 mmol) and [1,1′-biphenyl]-4-ylboronic acid (10.1 g, 50.8 mmol) were added to 300 ml of THF under a nitrogen atmosphere, and the mixture was stirred and refluxed. Then, potassium carbonate (20 g, 145.1 mmol) was dissolved in 60 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.5 g of subDF-2 (yield 51%, MS: [M+H]+=428).

subDF-2 (15 g, 35.1 mmol) and amine29 (18.1 g, 36.8 mmol) were added to 300 ml of THF under a nitrogen atmosphere, and the mixture was stirred and refluxed. Then, potassium carbonate (14.5 g, 105.2 mmol) was dissolved in 44 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.3 g of Compound 2-30 (yield 59%, MS: [M+H]+=839).

### EXAMPLES AND COMPARATIVE EXAMPLES

### Comparative Example A

A glass substrate on which ITO (indium tin oxide) was coated as a thin film to a thickness of 1000 Å was put into distilled water in which a detergent was dissolved, and ultrasonically cleaned. At this time, a product manufactured by Fischer Co. was used as the detergent, and distilled water filtered twice using a filter manufactured by Millipore Co. was used as the distilled water. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. Then, the substrate was cleaned for 5 minutes using oxygen plasma and then transferred to a vacuum depositor.

On the prepared ITO transparent electrode, the following Compound HI-1 was formed to a thickness of 1150 Å while the following Compound A-1 was p-doped at a concentration of 1.5% to form a hole injection layer. On the hole injection layer, the following Compound HT-1 was vacuum-deposited to form a hole transport layer having a thickness of 800 Å. Then, on the hole transport layer, the following Compound EB-1 was vacuum-deposited to form an electron blocking layer having a thickness of 150 Å. Then, on the EB-1 deposited film, the following Compound RH-1 and Compound Dp-7 were vacuum-deposited at a weight ratio of 98:2 to form a red light emitting layer having a thickness of 400 Å. On the light emitting layer, the following Compound HB-1 was vacuum-deposited to form a hole blocking layer having a thickness of 30 Å. On the hole blocking layer, the following Compound ET-1 and the following Compound LiQ were vacuum-deposited at a weight ratio of 2:1 to form an electron injection and transport layer having a thickness of 300 Å. On the electron injection and transport layer, lithium fluoride (LiF) and aluminum were sequentially deposited to a thickness of 12 Å and 1000 Å, respectively, to form a cathode.

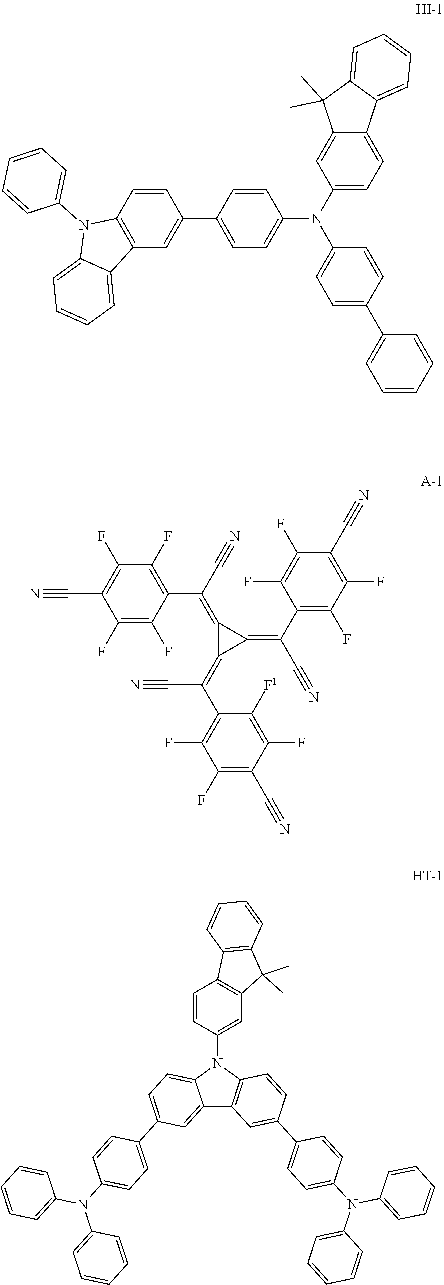

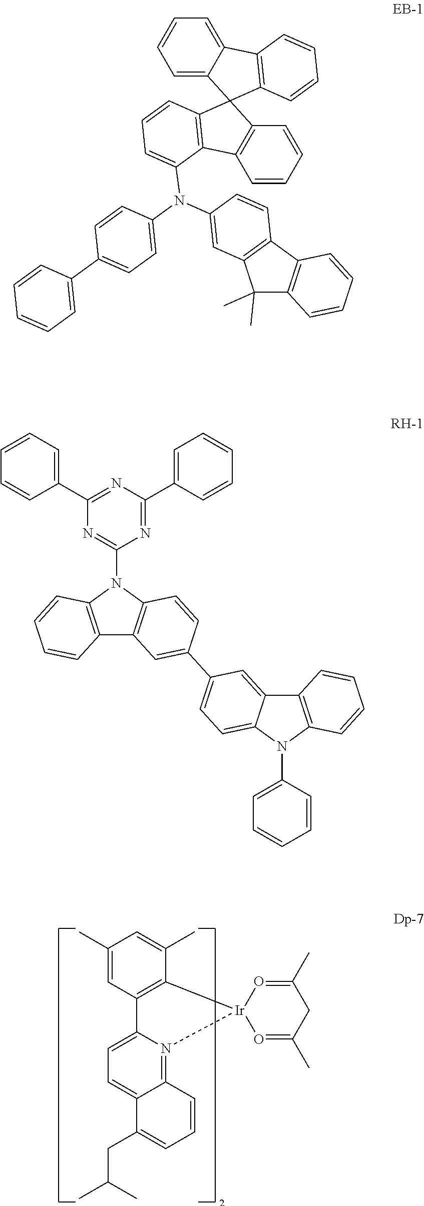

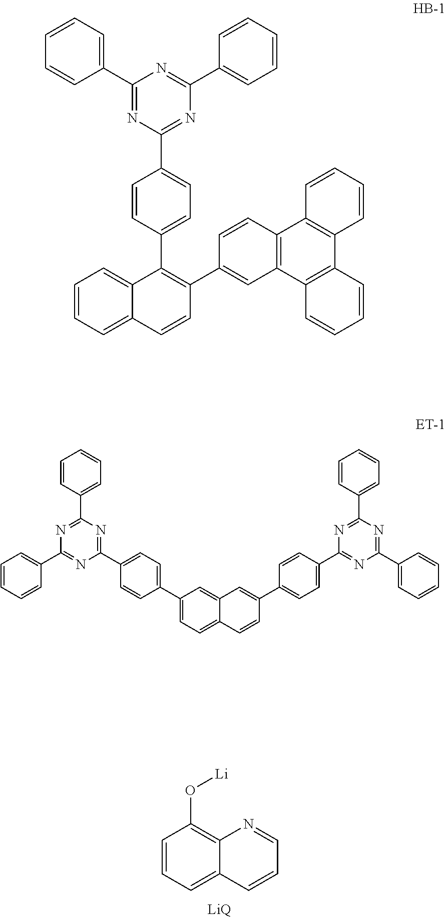

In the above process, the deposition rate of the organic material was maintained at 0.4 to 0.7 Å/sec, the deposition rate of lithium fluoride of the cathode was maintained at 0.3 Å/sec, and the deposition rate of aluminum was maintained at 2 Å/sec. In addition, the degree of vacuum during the deposition was maintained at 2×10⁻⁷ to 5×10⁻⁶ torr, thereby manufacturing an organic light emitting device.

### Examples 1 to 17

An organic light emitting device was manufactured in the same manner as in Comparative Example A, except that the compound shown in Table 1 was used instead of Compound RH-1 as a host in the organic light emitting device of Comparative Example A.

### Comparative Examples 1 to 7

An organic light emitting device was manufactured in the same manner as in Comparative Example A, except that the compound shown in Table 1 was used instead of Compound RH-1 as a host in the organic light emitting device of Comparative Example A. Compounds B-8 to B-14 listed in Table 1 are as follows.

### Examples 18 to 47

An organic light emitting device was manufactured in the same manner as in Comparative Example A, except that the compound shown in Table 2 was used as an electron blocking layer material instead of Compound EB-1 in the organic light emitting device of Comparative Example A.

### Comparative Examples 8 to 14

An organic light emitting device was manufactured in the same manner as in Comparative Example A, except that the compound shown in Table 2 was used as an electron blocking layer material instead of Compound EB-1 in the organic light emitting device of Comparative Example A. Compounds B-1 to B-7 listed in Table 2 are as follows.

### Examples 48 to 119

An organic light emitting device was manufactured in the same manner as in Comparative Example A, except that the first host and the second host described in Table 3 were used at a weight ratio of 1:1 instead of Compound RH-1 as a host in the organic light emitting device of Comparative Example A.

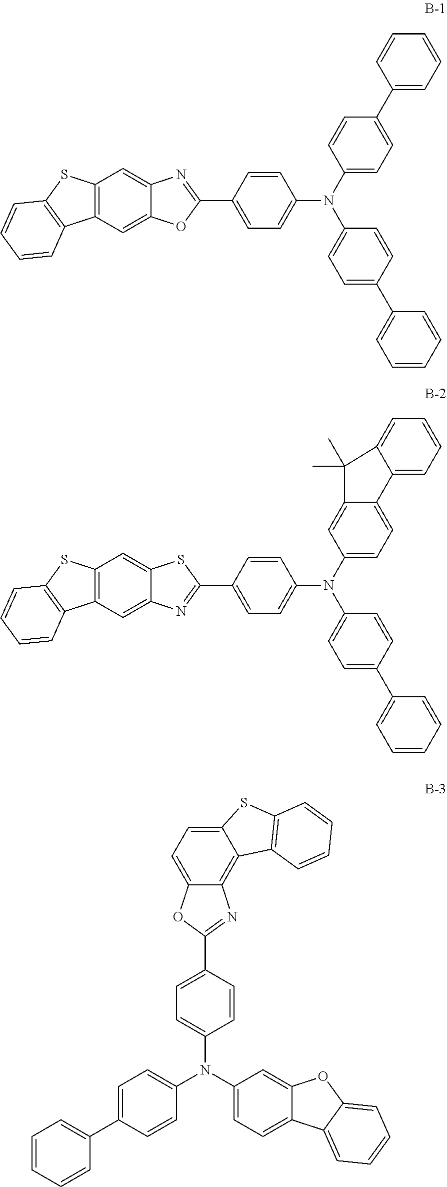 [SMILES: c1ccc(-c2ccc(N(c3ccc(-c4ccccc4)cc3)c3ccc(-c4nc5cc6sc7ccccc7c6cc5o4)cc3)cc2)cc1 ; CC1(C)c2ccccc2-c2ccc(N(c3ccc(-c4ccccc4)cc3)c3ccc(-c4nc5cc6c(cc5s4)sc4ccccc46)cc3)cc21 ; c1ccc(-c2ccc(N(c3ccc(-c4nc5c(ccc6sc7ccccc7c65)o4)cc3)c3ccc4c(c3)oc3ccccc34)cc2)cc1]

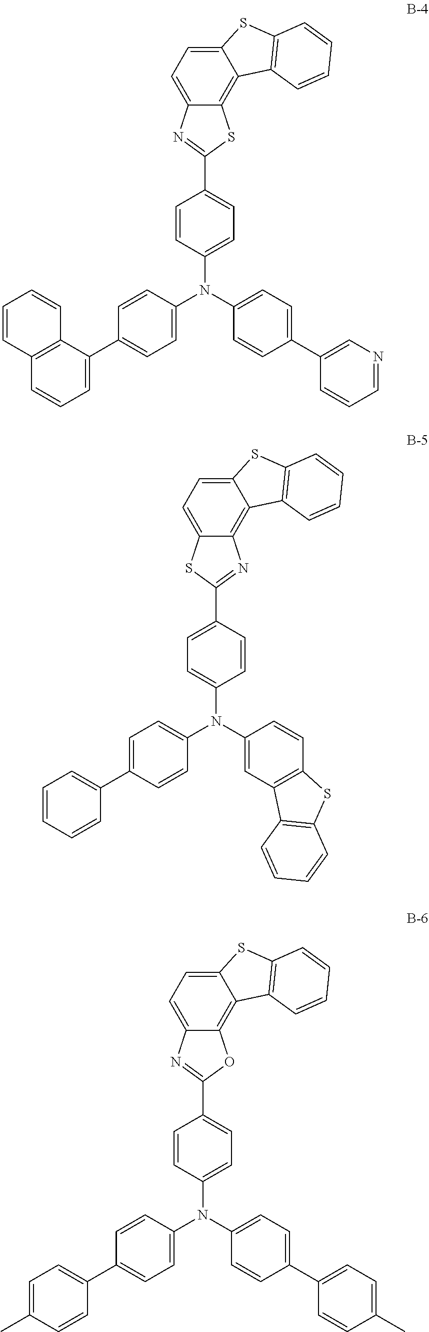

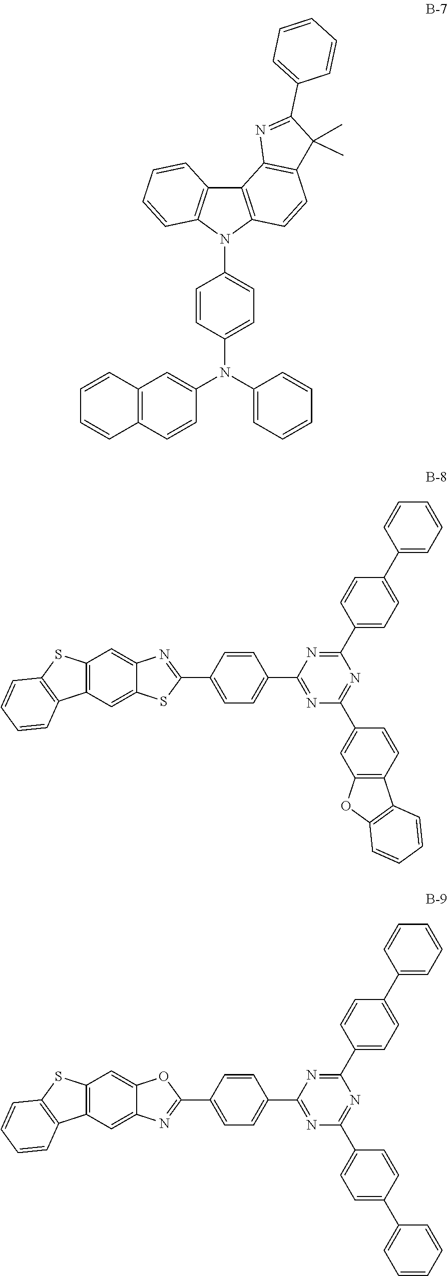 [SMILES: CC1(C)C(c2ccccc2)=Nc2c1ccc1c2c2ccccc2n1-c1ccc(N(c2ccccc2)c2ccc3ccccc3c2)cc1 ; c1ccc(-c2ccc(-c3nc(-c4ccc(-c5nc6cc7sc8ccccc8c7cc6s5)cc4)nc(-c4ccc5c(c4)oc4ccccc45)n3)cc2)cc1 ; c1ccc(-c2ccc(-c3nc(-c4ccc(-c5ccccc5)cc4)nc(-c4ccc(-c5nc6cc7c(cc6o5)sc5ccccc57)cc4)n3)cc2)cc1]

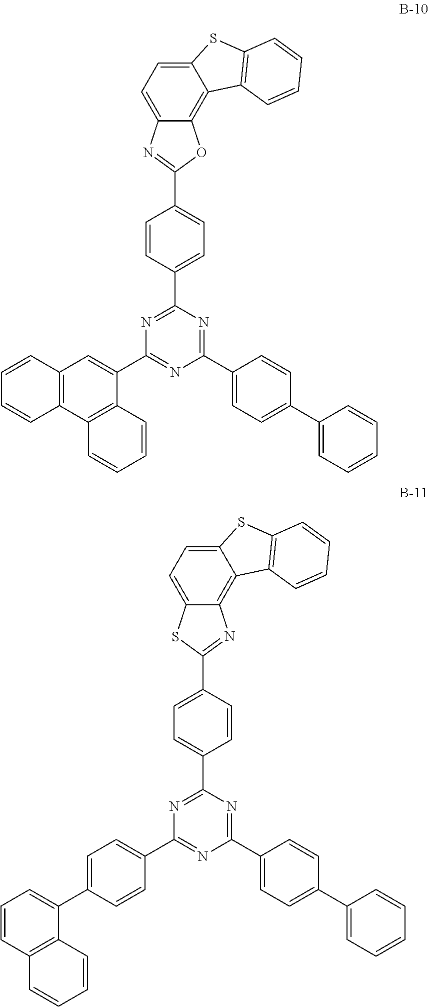 [SMILES: c1ccc(-c2ccc(-c3nc(-c4ccc(-c5nc6ccc7sc8ccccc8c7c6o5)cc4)nc(-c4cc5ccccc5c5ccccc45)n3)cc2)cc1 ; c1ccc(-c2ccc(-c3nc(-c4ccc(-c5nc6c(ccc7sc8ccccc8c76)s5)cc4)nc(-c4ccc(-c5cccc6ccccc56)cc4)n3)cc2)cc1]

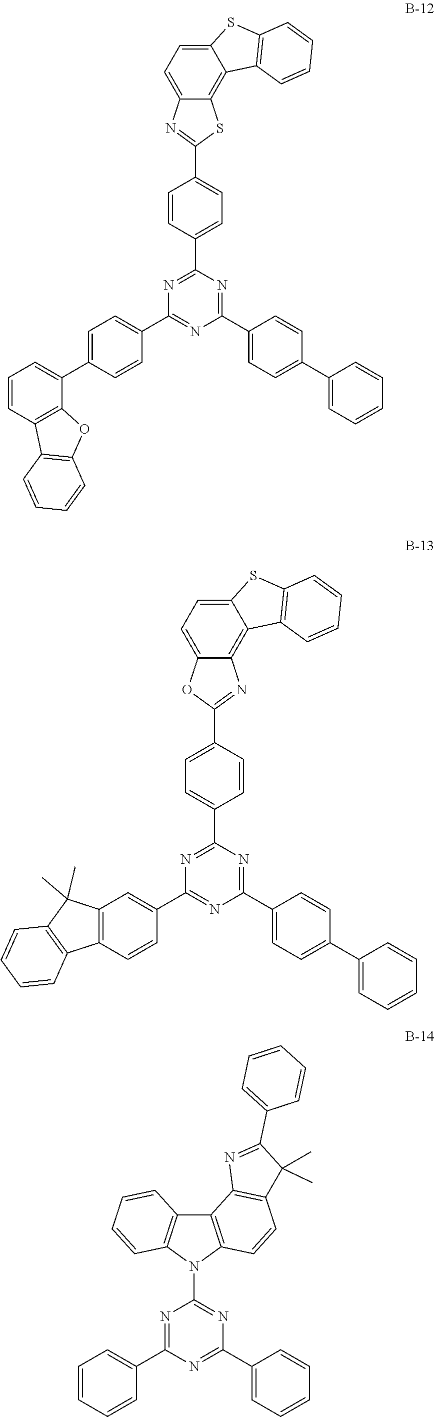 [SMILES: c1ccc(-c2ccc(-c3nc(-c4ccc(-c5nc6ccc7sc8ccccc8c7c6s5)cc4)nc(-c4ccc(-c5cccc6c5oc5ccccc56)cc4)n3)cc2)cc1 ; CC1(C)c2ccccc2-c2ccc(-c3nc(-c4ccc(-c5ccccc5)cc4)nc(-c4ccc(-c5nc6c(ccc7sc8ccccc8c76)o5)cc4)n3)cc21 ; CC1(C)C(c2ccccc2)=Nc2c1ccc1c2c2ccccc2n1-c1nc(-c2ccccc2)nc(-c2ccccc2)n1]

### Experimental Example

For the organic light emitting devices prepared in Examples 1 to 119, Comparative Example A and Comparative Examples 1 to 14, the voltage, efficiency, and lifespan were measured by applying a current (15 mA/cm²), and the results are shown in Tables 1 to 3 below. The lifespan (T95) means the time taken until the initial luminance (7,000 nit) decreases to 95%.

TABLE 1

| | | Driving | Effi- | Lifespan | Emis- |
|---|---|---|---|---|---|
| | | voltage | ciency | T95 | sion |
| Category | Host | (V) | (cd/A) | (hr) | color |
| Comparative | Compound RH-1 | 3.91 | 16.54 | 113 | Red |
| Example A | | | | | |
| Example 1 | Compound 1-1 | 3.72 | 18.09 | 162 | Red |
| Example 2 | Compound 1-2 | 3.71 | 18.15 | 161 | Red |
| Example 3 | Compound 1-3 | 3.68 | 17.91 | 160 | Red |
| Example 4 | Compound 1-4 | 3.78 | 17.64 | 157 | Red |
| Example 5 | Compound 1-5 | 3.70 | 18.28 | 166 | Red |
| Example 6 | Compound 1-6 | 3.71 | 18.34 | 165 | Red |
| Example 7 | Compound 1-7 | 3.72 | 17.94 | 155 | Red |
| Example 8 | Compound 1-8 | 3.68 | 18.24 | 162 | Red |
| Example 9 | Compound 1-9 | 3.77 | 18.20 | 169 | Red |
| Example 10 | Compound 1-10 | 3.71 | 18.21 | 171 | Red |
| Example 11 | Compound 1-11 | 3.83 | 18.59 | 201 | Red |
| Example 12 | Compound 1-12 | 3.84 | 18.92 | 199 | Red |
| Example 13 | Compound 1-13 | 3.53 | 19.77 | 219 | Red |
| Example 14 | Compound 1-14 | 3.53 | 19.98 | 210 | Red |
| Example 15 | Compound 1-15 | 3.59 | 19.62 | 226 | Red |
| Example 16 | Compound 1-16 | 3.55 | 20.48 | 217 | Red |
| Example 17 | Compound 1-17 | 3.61 | 19.70 | 212 | Red |
| Comparative | Compound B-8 | 4.06 | 15.55 | 127 | Red |
| Example 1 | | | | | |
| Comparative | Compound B-9 | 4.11 | 15.53 | 122 | Red |
| Example 2 | | | | | |
| Comparative | Compound B-10 | 4.11 | 15.68 | 118 | Red |
| Example 3 | | | | | |
| Comparative | Compound B-11 | 4.19 | 15.31 | 111 | Red |
| Example 4 | | | | | |
| Comparative | Compound B-12 | 4.29 | 14.71 | 113 | Red |
| Example 5 | | | | | |
| Comparative | Compound B-13 | 4.28 | 15.01 | 94 | Red |
| Example 6 | | | | | |
| Comparative | Compound B-14 | 4.07 | 15.63 | 117 | Red |
| Example 7 | | | | | |

TABLE 2

| | Electron | Driving | Effi- | Lifespan | Emis- |
|---|---|---|---|---|---|
| | blocking | voltage | ciency | T95 | sion |
| Category | layer | (V) | (cd/A) | (hr) | color |
| Example 18 | Compound 2-1 | 3.67 | 17.26 | 172 | Red |
| Example 19 | Compound 2-2 | 3.71 | 17.44 | 168 | Red |
| Example 20 | Compound 2-3 | 3.71 | 17.64 | 186 | Red |
| Example 21 | Compound 2-4 | 3.69 | 17.64 | 175 | Red |
| Example 22 | Compound 2-5 | 3.61 | 17.59 | 184 | Red |
| Example 23 | Compound 2-6 | 3.68 | 17.65 | 182 | Red |
| Example 24 | Compound 2-7 | 3.70 | 17.40 | 171 | Red |
| Example 25 | Compound 2-8 | 3.77 | 18.35 | 209 | Red |
| Example 26 | Compound 2-9 | 3.85 | 18.81 | 184 | Red |
| Example 27 | Compound 2-10 | 3.81 | 18.82 | 185 | Red |
| Example 28 | Compound 2-11 | 3.86 | 18.51 | 202 | Red |
| Example 29 | Compound 2-12 | 3.75 | 18.57 | 198 | Red |
| Example 30 | Compound 2-13 | 3.80 | 18.42 | 204 | Red |
| Example 31 | Compound 2-14 | 3.85 | 18.57 | 208 | Red |
| Example 32 | Compound 2-15 | 3.72 | 17.84 | 157 | Red |
| Example 33 | Compound 2-16 | 3.77 | 17.59 | 171 | Red |
| Example 34 | Compound 2-17 | 3.79 | 17.52 | 169 | Red |
| Example 35 | Compound 2-18 | 3.79 | 17.81 | 160 | Red |
| Example 36 | Compound 2-19 | 3.79 | 18.06 | 164 | Red |
| Example 37 | Compound 2-20 | 3.76 | 17.92 | 156 | Red |
| Example 38 | Compound 2-21 | 3.62 | 20.66 | 214 | Red |
| Example 39 | Compound 2-22 | 3.63 | 19.99 | 218 | Red |
| Example 40 | Compound 2-23 | 3.58 | 19.41 | 219 | Red |
| Example 41 | Compound 2-24 | 3.53 | 19.23 | 205 | Red |
| Example 42 | Compound 2-25 | 3.54 | 20.27 | 206 | Red |
| Example 43 | Compound 2-26 | 3.55 | 20.40 | 215 | Red |
| Example 44 | Compound 2-27 | 3.61 | 20.72 | 218 | Red |
| Example 45 | Compound 2-28 | 3.53 | 19.98 | 210 | Red |
| Example 46 | Compound 2-29 | 3.58 | 19.52 | 214 | Red |
| Example 47 | Compound 2-30 | 3.57 | 20.96 | 221 | Red |
| Comparative | Compound B-1 | 4.18 | 14.85 | 96 | Red |
| Example 8 | | | | | |
| Comparative | Compound B-2 | 4.24 | 14.92 | 112 | Red |
| Example 9 | | | | | |
| Comparative | Compound B-3 | 4.28 | 15.34 | 115 | Red |
| Example 10 | | | | | |
| Comparative | Compound B-4 | 4.16 | 14.95 | 91 | Red |
| Example 11 | | | | | |
| Comparative | Compound B-5 | 4.22 | 15.31 | 107 | Red |
| Example 12 | | | | | |
| Comparative | Compound B-6 | 4.12 | 15.94 | 121 | Red |
| Example 13 | | | | | |
| Comparative | Compound B-7 | 4.05 | 15.60 | 124 | Red |
| Example 14 | | | | | |

TABLE 3

| | | | Driving | | Lifespan | |
|---|---|---|---|---|---|---|
| | | | voltage | Efficiency | T95 | Emission |
| Category | First host | Second host | (V) | (cd/A) | (hr) | color |
| Example 48 | Compound 1-1 | Compound 2-1 | 3.45 | 22.22 | 247 | Red |
| Example 49 | Compound 1-1 | Compound 2-4 | 3.50 | 22.27 | 244 | Red |
| Example 50 | Compound 1-1 | Compound 2-10 | 3.43 | 22.02 | 237 | Red |
| Example 51 | Compound 1-1 | Compound 2-15 | 3.48 | 22.13 | 244 | Red |
| Example 52 | Compound 1-1 | Compound 2-22 | 3.42 | 22.65 | 238 | Red |
| Example 53 | Compound 1-1 | Compound 2-26 | 3.45 | 22.35 | 239 | Red |
| Example 54 | Compound 1-3 | Compound 2-2 | 3.50 | 22.07 | 245 | Red |
| Example 55 | Compound 1-3 | Compound 2-5 | 3.47 | 22.18 | 235 | Red |
| Example 56 | Compound 1-3 | Compound 2-11 | 3.45 | 22.06 | 249 | Red |
| Example 57 | Compound 1-3 | Compound 2-16 | 3.50 | 22.70 | 248 | Red |
| Example 58 | Compound 1-3 | Compound 2-23 | 3.62 | 22.16 | 212 | Red |
| Example 59 | Compound 1-3 | Compound 2-27 | 3.37 | 21.63 | 255 | Red |
| Example 60 | Compound 1-4 | Compound 2-3 | 3.32 | 21.99 | 256 | Red |
| Example 61 | Compound 1-4 | Compound 2-6 | 3.31 | 22.07 | 243 | Red |
| Example 62 | Compound 1-4 | Compound 2-12 | 3.33 | 21.86 | 251 | Red |
| Example 63 | Compound 1-4 | Compound 2-17 | 3.32 | 21.88 | 256 | Red |
| Example 64 | Compound 1-4 | Compound 2-24 | 3.37 | 22.16 | 248 | Red |
| Example 65 | Compound 1-4 | Compound 2-28 | 3.32 | 21.96 | 245 | Red |
| Example 66 | Compound 1-5 | Compound 2-1 | 3.31 | 22.20 | 256 | Red |
| Example 67 | Compound 1-5 | Compound 2-4 | 3.30 | 22.01 | 255 | Red |
| Example 68 | Compound 1-5 | Compound 2-13 | 3.34 | 21.65 | 250 | Red |
| Example 69 | Compound 1-5 | Compound 2-18 | 3.40 | 20.84 | 206 | Red |
| Example 70 | Compound 1-5 | Compound 2-25 | 3.45 | 23.20 | 275 | Red |
| Example 71 | Compound 1-5 | Compound 2-29 | 3.50 | 23.13 | 270 | Red |
| Example 72 | Compound 1-8 | Compound 2-1 | 3.43 | 22.87 | 267 | Red |
| Example 73 | Compound 1-8 | Compound 2-4 | 3.48 | 23.15 | 264 | Red |
| Example 74 | Compound 1-8 | Compound 2-10 | 3.42 | 23.12 | 262 | Red |
| Example 75 | Compound 1-8 | Compound 2-15 | 3.45 | 22.83 | 268 | Red |
| Example 76 | Compound 1-8 | Compound 2-22 | 3.50 | 23.12 | 262 | Red |
| Example 77 | Compound 1-8 | Compound 2-26 | 3.47 | 22.99 | 262 | Red |
| Example 78 | Compound 1-9 | Compound 2-2 | 3.45 | 23.20 | 275 | Red |
| Example 79 | Compound 1-9 | Compound 2-5 | 3.50 | 22.95 | 272 | Red |
| Example 80 | Compound 1-9 | Compound 2-11 | 3.44 | 24.12 | 285 | Red |
| Example 81 | Compound 1-9 | Compound 2-16 | 3.37 | 23.71 | 289 | Red |
| Example 82 | Compound 1-9 | Compound 2-23 | 3.44 | 23.97 | 288 | Red |
| Example 83 | Compound 1-9 | Compound 2-27 | 3.35 | 24.15 | 286 | Red |
| Example 84 | Compound 1-10 | Compound 2-3 | 3.43 | 23.98 | 282 | Red |
| Example 85 | Compound 1-10 | Compound 2-6 | 3.36 | 24.44 | 274 | Red |
| Example 86 | Compound 1-10 | Compound 2-12 | 3.40 | 24.40 | 273 | Red |
| Example 87 | Compound 1-10 | Compound 2-17 | 3.44 | 23.73 | 270 | Red |
| Example 88 | Compound 1-10 | Compound 2-24 | 3.44 | 24.14 | 275 | Red |
| Example 89 | Compound 1-10 | Compound 2-28 | 3.38 | 23.76 | 287 | Red |
| Example 90 | Compound 1-13 | Compound 2-1 | 3.38 | 23.76 | 287 | Red |
| Example 91 | Compound 1-13 | Compound 2-4 | 3.45 | 23.20 | 275 | Red |
| Example 92 | Compound 1-13 | Compound 2-13 | 3.50 | 23.13 | 270 | Red |
| Example 93 | Compound 1-13 | Compound 2-18 | 3.45 | 23.00 | 275 | Red |
| Example 94 | Compound 1-13 | Compound 2-25 | 3.47 | 23.21 | 274 | Red |
| Example 95 | Compound 1-13 | Compound 2-29 | 3.47 | 22.94 | 267 | Red |
| Example 96 | Compound 1-14 | Compound 2-1 | 3.54 | 22.81 | 260 | Red |
| Example 97 | Compound 1-14 | Compound 2-4 | 3.52 | 23.18 | 262 | Red |
| Example 98 | Compound 1-14 | Compound 2-10 | 3.46 | 23.25 | 266 | Red |
| Example 99 | Compound 1-14 | Compound 2-15 | 3.55 | 22.80 | 272 | Red |
| Example 100 | Compound 1-14 | Compound 2-22 | 3.51 | 23.30 | 263 | Red |
| Example 101 | Compound 1-14 | Compound 2-26 | 3.49 | 22.97 | 274 | Red |
| Example 102 | Compound 1-15 | Compound 2-2 | 3.45 | 22.23 | 236 | Red |
| Example 103 | Compound 1-15 | Compound 2-5 | 3.47 | 22.84 | 243 | Red |
| Example 104 | Compound 1-15 | Compound 2-11 | 3.47 | 22.07 | 247 | Red |
| Example 105 | Compound 1-15 | Compound 2-16 | 3.54 | 22.63 | 242 | Red |
| Example 106 | Compound 1-15 | Compound 2-23 | 3.52 | 22.62 | 238 | Red |
| Example 107 | Compound 1-15 | Compound 2-27 | 3.46 | 22.73 | 242 | Red |
| Example 108 | Compound 1-16 | Compound 2-3 | 3.55 | 22.83 | 250 | Red |
| Example 109 | Compound 1-16 | Compound 2-6 | 3.51 | 22.41 | 245 | Red |
| Example 110 | Compound 1-16 | Compound 2-12 | 3.40 | 24.49 | 273 | Red |
| Example 111 | Compound 1-16 | Compound 2-17 | 3.44 | 24.03 | 284 | Red |
| Example 112 | Compound 1-16 | Compound 2-24 | 3.42 | 24.47 | 270 | Red |
| Example 113 | Compound 1-16 | Compound 2-28 | 3.39 | 23.86 | 285 | Red |
| Example 114 | Compound 1-17 | Compound 2-1 | 3.36 | 23.80 | 275 | Red |
| Example 115 | Compound 1-17 | Compound 2-4 | 3.36 | 24.22 | 290 | Red |
| Example 116 | Compound 1-17 | Compound 2-10 | 3.39 | 24.47 | 272 | Red |
| Example 117 | Compound 1-17 | Compound 2-15 | 3.43 | 24.19 | 281 | Red |
| Example 118 | Compound 1-17 | Compound 2-22 | 3.37 | 24.34 | 289 | Red |
| Example 119 | Compound 1-17 | Compound 2-26 | 3.42 | 24.49 | 272 | Red |

When a current was applied to the organic light emitting devices manufactured according to Examples 1 to 119 and Comparative Examples 1 to 14, the results shown in Tables 1 to 3 were obtained.

When the Compounds 1-1 to 1-17 of the present disclosure were used as a red host, it was confirmed that the driving voltage was lowered, and the efficiency and lifespan were increased compared to the case of using the Compounds of Comparative Examples as shown in Table 1. In addition, when the Compounds 2-1 to 2-30 of the present disclosure were used for an electronic blocking layer, it was confirmed that the driving voltage was lowered, and the efficiency and lifespan were increased compared to the case of using the Compounds of Comparative Examples as shown in Table 2.

In Table 3, when one of Compounds 1-1 to 1-17 was selected as a first host and one of Compounds 2-1 to 2-30 was selected as a second host, and they were co-deposited and used as a red host, it was confirmed that the driving voltage was lowered and the efficiency and lifespan were increased compared to the case of using a single-material host.

From the results shown in Tables 1 to 3, it can be confirmed that when the compound of Chemical Formula 1 is used as the host of a red light emitting layer or the electron blocking layer material in a red device, the driving voltage, luminous efficiency and lifespan of the organic light emitting device can be improved.

[DESCRIPTION OF SYMBOLS]

| | |
|---|---|
| 1: Substrate | 2: Anode |
| 3: Organic material layer | 4: Cathode |
| 5: Hole injection layer | 6: Hole transport layer |
| 7: Electron blocking layer | 8: Light emitting layer |
| 9: Hole blocking layer | 10: Electron transport layer |
| 11: Electron injection layer | 12: Electron injection and transport |

1. A compound of Chemical Formula 1: 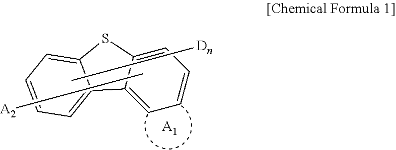
wherein in Chemical Formula 1:
A₁ represents Chemical Formula 1-a: 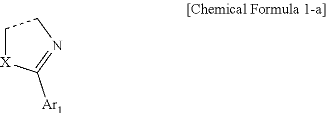
wherein in Chemical Formula 1-a;
the dotted line is fused with an adjacent ring,
X is O or S; and
Ar₁ is a substituted or unsubstituted C₆₋₆₀ aryl, or a substituted or unsubstituted C₂₋₆₀ heteroaryl containing at least one heteroatom selected from the group consisting of N, O and S; and
A₂ is a substituent of Chemical Formula 1-b or Chemical Formula 1-c; 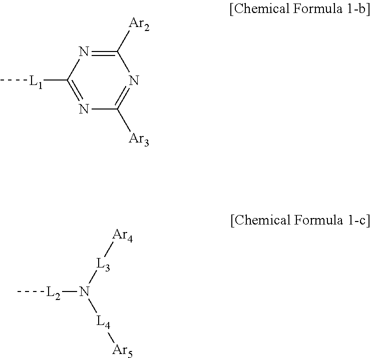
wherein in Chemical Formulae 1-b and 1-c;
L₁ to L₄ are each independently a single bond, a substituted or unsubstituted C₆₋₆₀ arylene, or a substituted or unsubstituted C₂₋₆₀ heteroarylene containing at least one heteroatom selected from the group consisting of N, O and S;
Ar₂ to Ar₅ are each independently a substituted or unsubstituted C₆₋₆₀ aryl, or substituted or unsubstituted C₂₋₆₀ heteroaryl containing at least one heteroatom selected from the group consisting of N, O and S;
D is deuterium; and
n is an integer of 0 to 5.

2. The compound of claim 1, wherein Chemical Formula 1 is any one of the following Chemical Formulae 1-1 to 1-4: 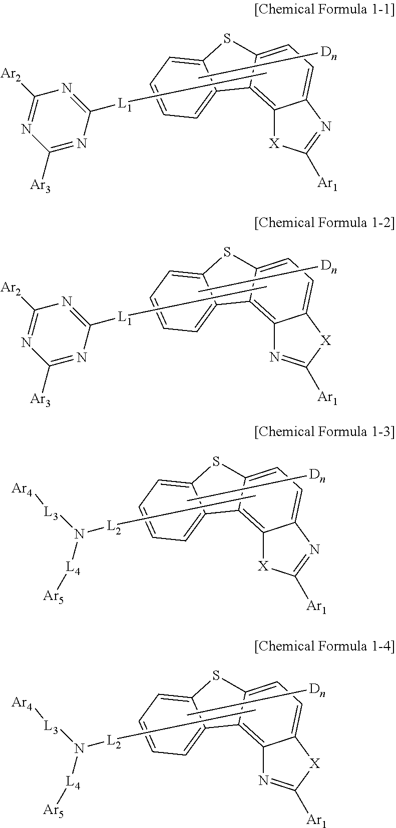
wherein in Chemical Formulae 1-1 to 1-4, X, L₁ to L₄, Ar₁ to Ar₅, D, and n are as defined in claim 1.

3. The compound of claim 1, wherein L₁ and L₂ are each independently a single bond, phenylene, biphenyldiyl, or naphthalenediyl.

4. The compound of claim 1, wherein Ar₁ is phenyl, biphenylyl, naphthyl, dibenzofuranyl or dibenzothiophenyl.

5. The compound of claim 1, wherein Ar₂ and Ar₃ are each independently phenyl, biphenylyl, naphthyl, phenylnaphthyl, naphthylphenyl, dibenzofuranyl, dibenzothiophenyl, or phenanthrenyl.

6. The compound of claim 1, wherein L₃ and L₄ are each independently a single bond, phenylene, biphenyldiyl, or naphthalenediyl.

7. The compound of claim 1, wherein Ar₄ and Ar₅ are each independently phenyl, biphenylyl, terphenylyl, naphthyl, naphthylphenyl, phenylnaphthyl, phenanthrenyl, 9-phenylcarbazolyl, dibenzofuranyl, or dibenzothiophenyl.

8. The compound of claim 1, wherein the compound of Chemical Formula 1 is any one compound selected from the group consisting of:

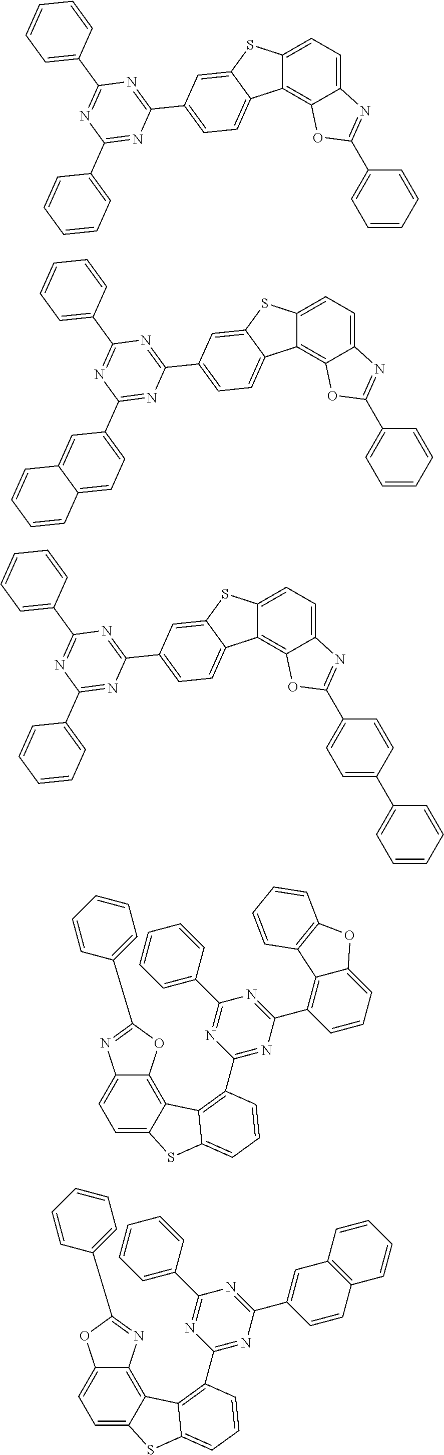

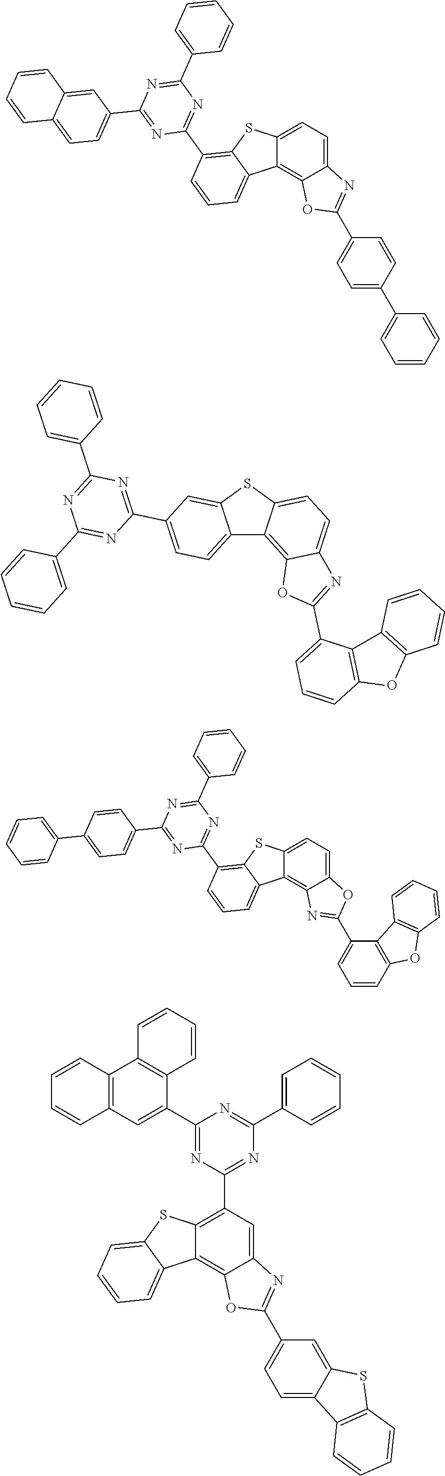 [SMILES: c1ccc(-c2ccc(-c3nc4ccc5sc6c(-c7nc(-c8ccccc8)nc(-c8ccc9ccccc9c8)n7)cccc6c5c4o3)cc2)cc1 ; c1ccc(-c2nc(-c3ccccc3)nc(-c3ccc4c(c3)sc3ccc5nc(-c6cccc7oc8ccccc8c67)oc5c34)n2)cc1 ; c1ccc(-c2ccc(-c3nc(-c4ccccc4)nc(-c4cccc5c4sc4ccc6oc(-c7cccc8oc9ccccc9c78)nc6c45)n3)cc2)cc1 ; c1ccc(-c2nc(-c3cc4ccccc4c4ccccc34)nc(-c3cc4nc(-c5ccc6c(c5)sc5ccccc56)oc4c4c3sc3ccccc34)n2)cc1]

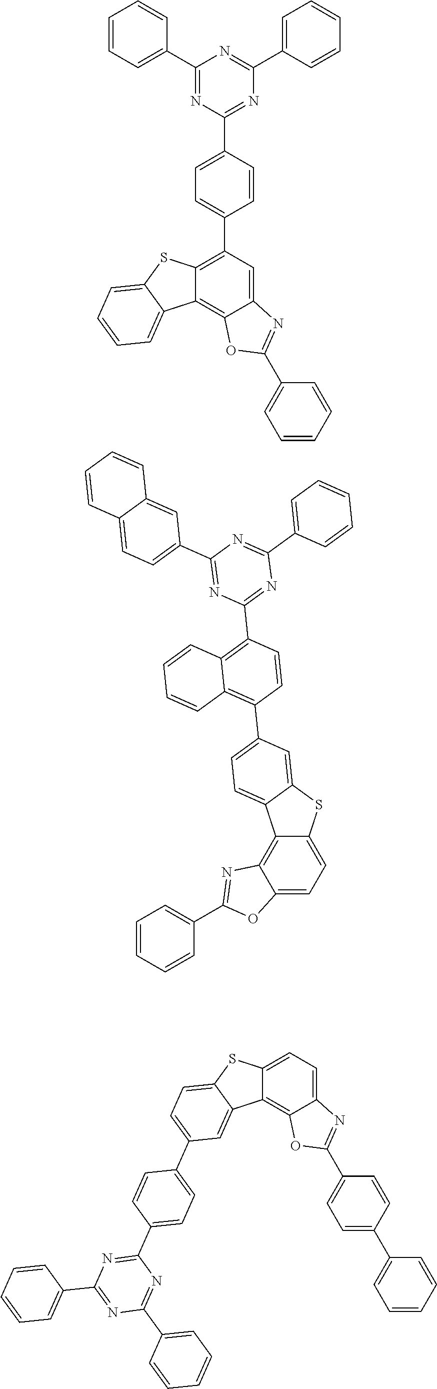

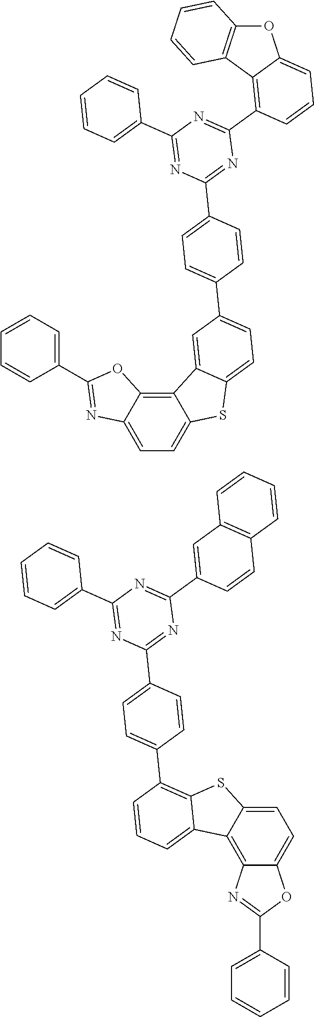 [SMILES: c1ccc(-c2nc(-c3ccc(-c4ccc5sc6ccc7nc(-c8ccccc8)oc7c6c5c4)cc3)nc(-c3cccc4oc5ccccc5c34)n2)cc1 ; c1ccc(-c2nc(-c3ccc(-c4cccc5c4sc4ccc6oc(-c7ccccc7)nc6c45)cc3)nc(-c3ccc4ccccc4c3)n2)cc1]

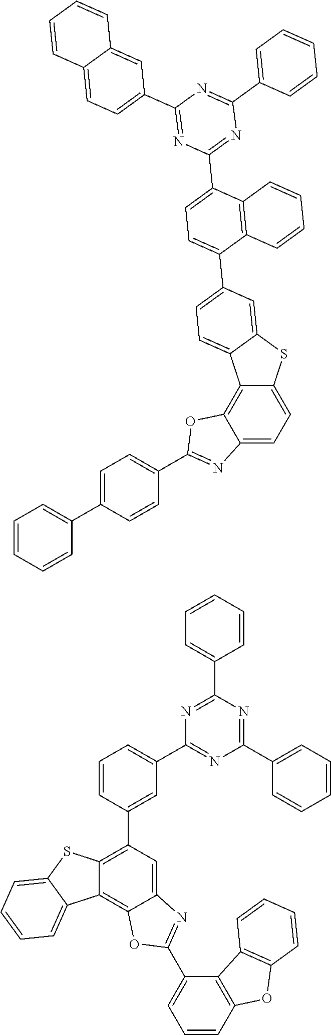 [SMILES: c1ccc(-c2ccc(-c3nc4ccc5sc6cc(-c7ccc(-c8nc(-c9ccccc9)nc(-c9ccc%10ccccc%10c9)n8)c8ccccc78)ccc6c5c4o3)cc2)cc1 ; c1ccc(-c2nc(-c3ccccc3)nc(-c3cccc(-c4cc5nc(-c6cccc7oc8ccccc8c67)oc5c5c4sc4ccccc45)c3)n2)cc1]

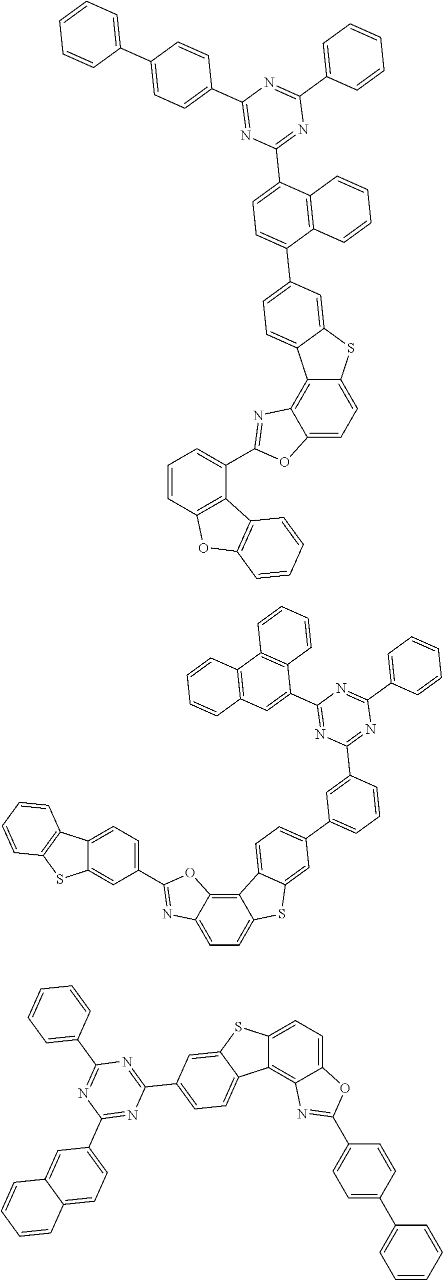

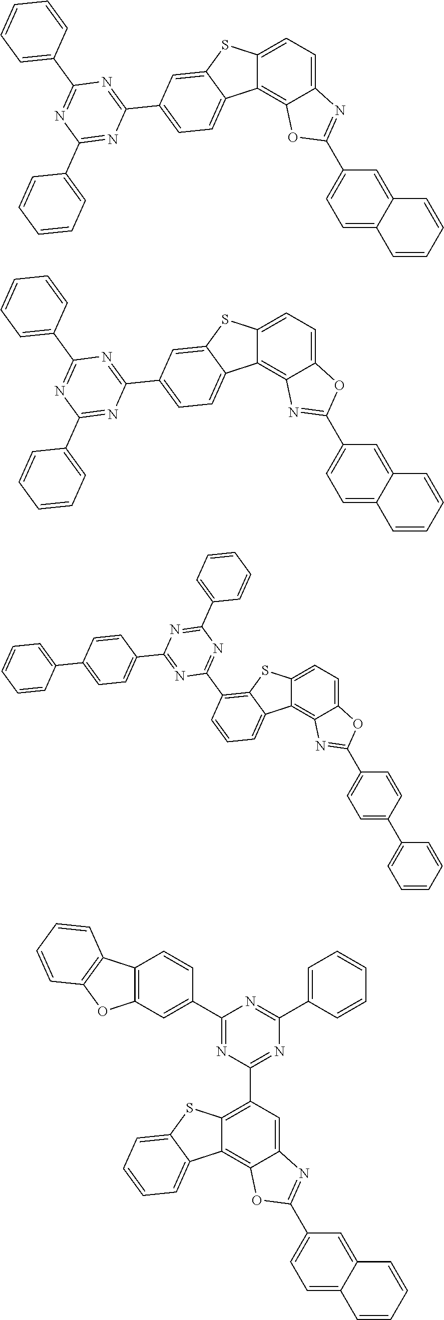

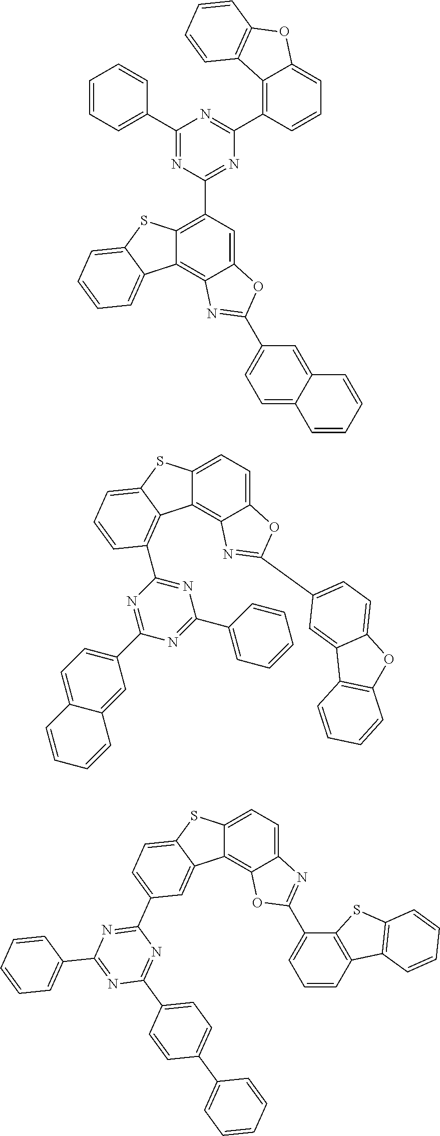 [SMILES: c1ccc(-c2nc(-c3cc4oc(-c5ccc6ccccc6c5)nc4c4c3sc3ccccc34)nc(-c3cccc4oc5ccccc5c34)n2)cc1 ; c1ccc(-c2nc(-c3ccc4ccccc4c3)nc(-c3cccc4sc5ccc6oc(-c7ccc8oc9ccccc9c8c7)nc6c5c34)n2)cc1 ; c1ccc(-c2ccc(-c3nc(-c4ccccc4)nc(-c4ccc5sc6ccc7nc(-c8cccc9c8sc8ccccc89)oc7c6c5c4)n3)cc2)cc1]

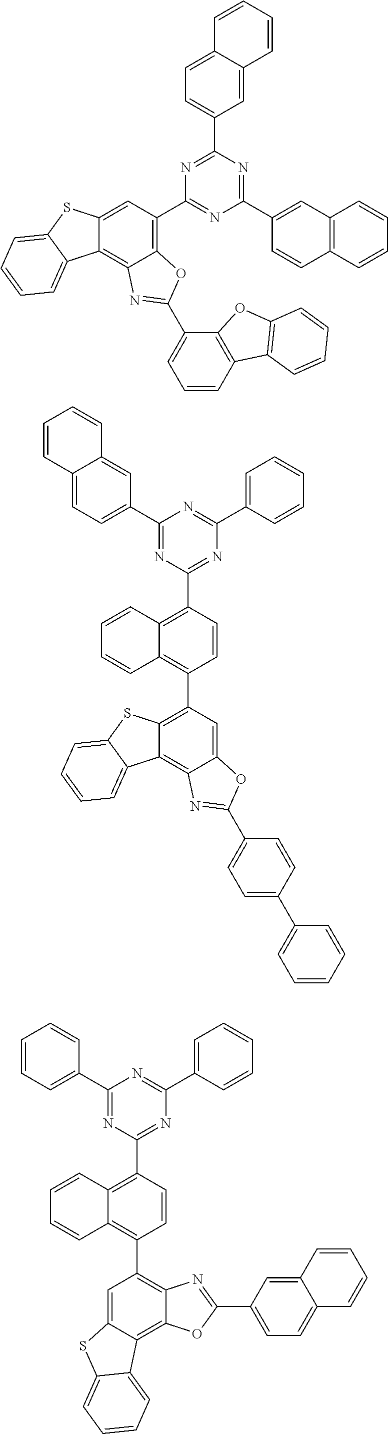 [SMILES: c1ccc2cc(-c3nc(-c4ccc5ccccc5c4)nc(-c4cc5sc6ccccc6c5c5nc(-c6cccc7c6oc6ccccc67)oc45)n3)ccc2c1 ; c1ccc(-c2ccc(-c3nc4c(cc(-c5ccc(-c6nc(-c7ccccc7)nc(-c7ccc8ccccc8c7)n6)c6ccccc56)c5sc6ccccc6c54)o3)cc2)cc1 ; c1ccc(-c2nc(-c3ccccc3)nc(-c3ccc(-c4cc5sc6ccccc6c5c5oc(-c6ccc7ccccc7c6)nc45)c4ccccc34)n2)cc1]

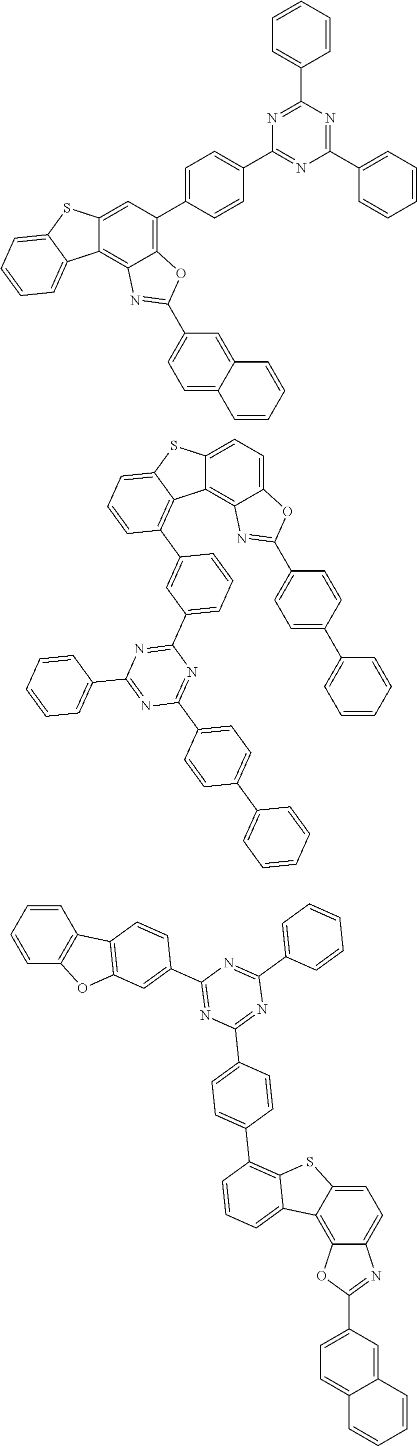

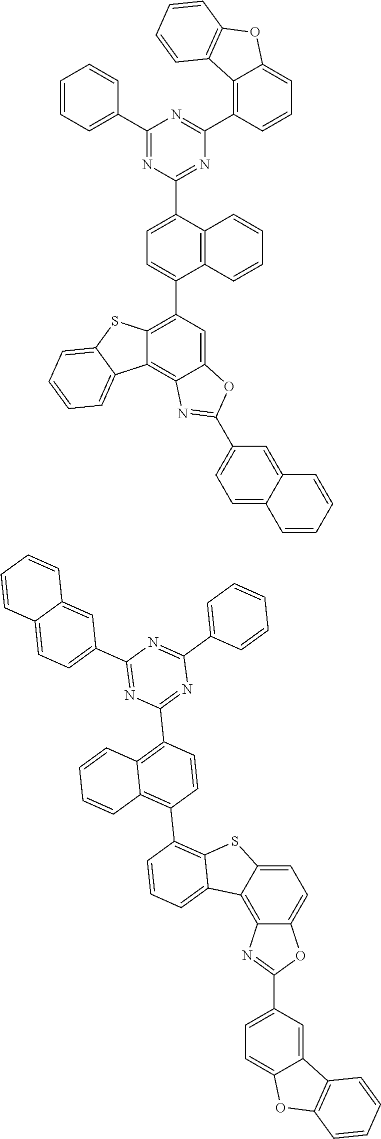 [SMILES: c1ccc(-c2nc(-c3ccc(-c4cc5oc(-c6ccc7ccccc7c6)nc5c5c4sc4ccccc45)c4ccccc34)nc(-c3cccc4oc5ccccc5c34)n2)cc1 ; c1ccc(-c2nc(-c3ccc4ccccc4c3)nc(-c3ccc(-c4cccc5c4sc4ccc6oc(-c7ccc8oc9ccccc9c8c7)nc6c45)c4ccccc34)n2)cc1]

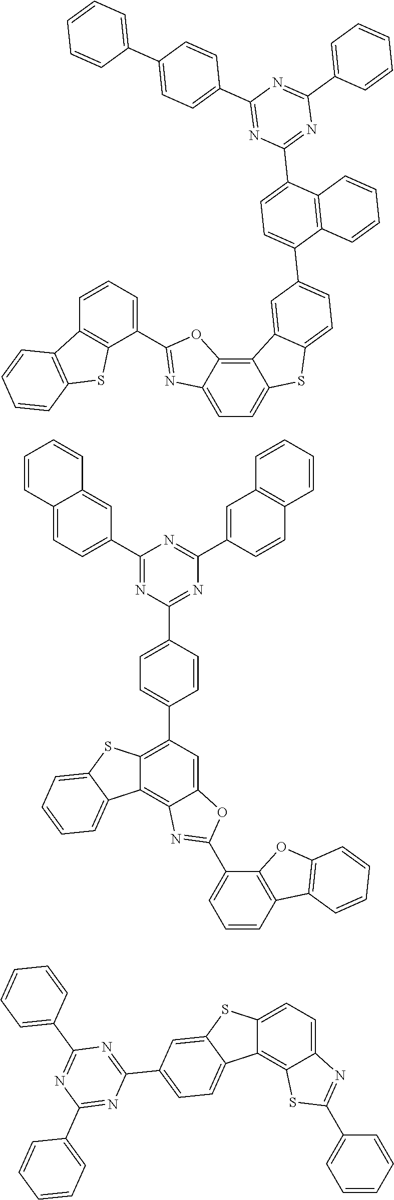

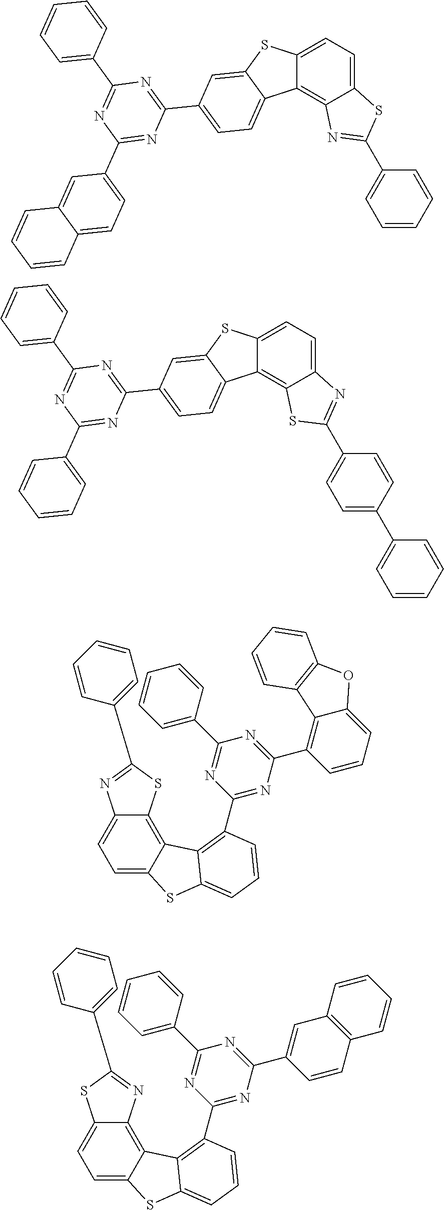

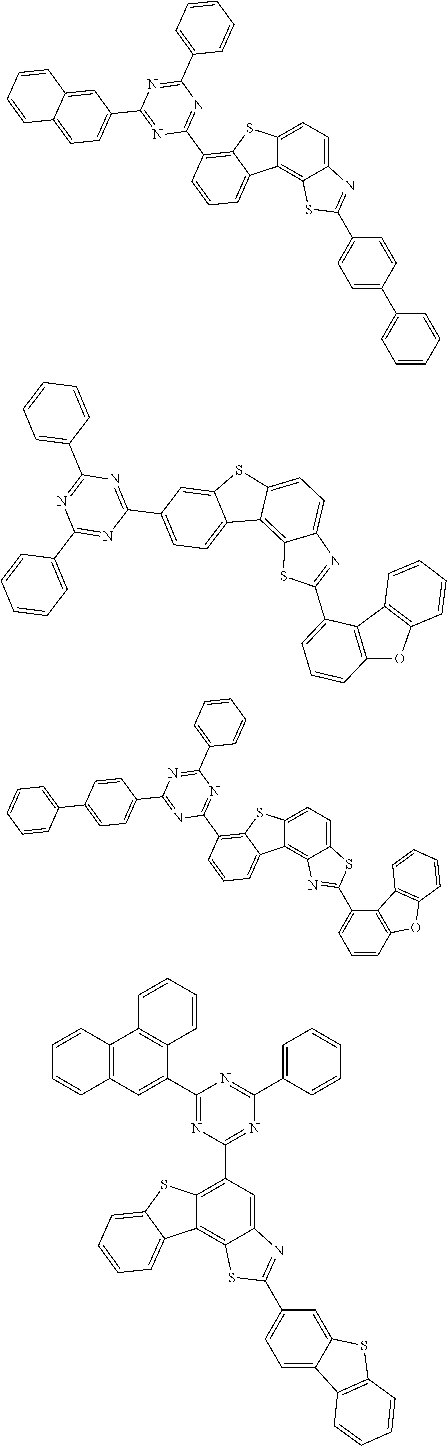 [SMILES: c1ccc(-c2ccc(-c3nc4ccc5sc6c(-c7nc(-c8ccccc8)nc(-c8ccc9ccccc9c8)n7)cccc6c5c4s3)cc2)cc1 ; c1ccc(-c2nc(-c3ccccc3)nc(-c3ccc4c(c3)sc3ccc5nc(-c6cccc7oc8ccccc8c67)sc5c34)n2)cc1 ; c1ccc(-c2ccc(-c3nc(-c4ccccc4)nc(-c4cccc5c4sc4ccc6sc(-c7cccc8oc9ccccc9c78)nc6c45)n3)cc2)cc1 ; c1ccc(-c2nc(-c3cc4ccccc4c4ccccc34)nc(-c3cc4nc(-c5ccc6c(c5)sc5ccccc56)sc4c4c3sc3ccccc34)n2)cc1]

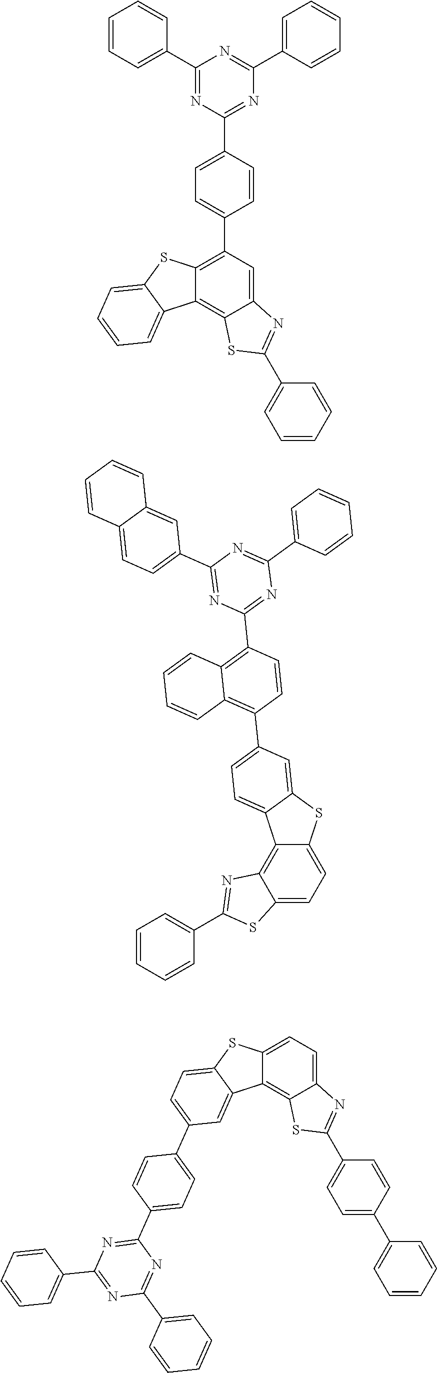 [SMILES: c1ccc(-c2nc(-c3ccccc3)nc(-c3ccc(-c4cc5nc(-c6ccccc6)sc5c5c4sc4ccccc45)cc3)n2)cc1 ; c1ccc(-c2nc(-c3ccc4ccccc4c3)nc(-c3ccc(-c4ccc5c(c4)sc4ccc6sc(-c7ccccc7)nc6c45)c4ccccc34)n2)cc1 ; c1ccc(-c2ccc(-c3nc4ccc5sc6ccc(-c7ccc(-c8nc(-c9ccccc9)nc(-c9ccccc9)n8)cc7)cc6c5c4s3)cc2)cc1]

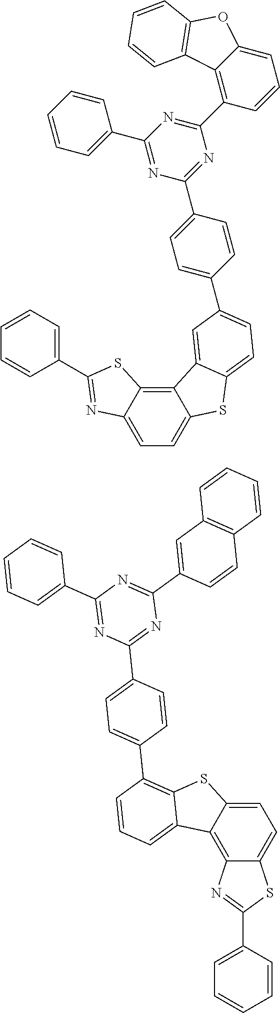 [SMILES: c1ccc(-c2nc(-c3ccc(-c4ccc5sc6ccc7nc(-c8ccccc8)sc7c6c5c4)cc3)nc(-c3cccc4oc5ccccc5c34)n2)cc1 ; c1ccc(-c2nc(-c3ccc(-c4cccc5c4sc4ccc6sc(-c7ccccc7)nc6c45)cc3)nc(-c3ccc4ccccc4c3)n2)cc1]

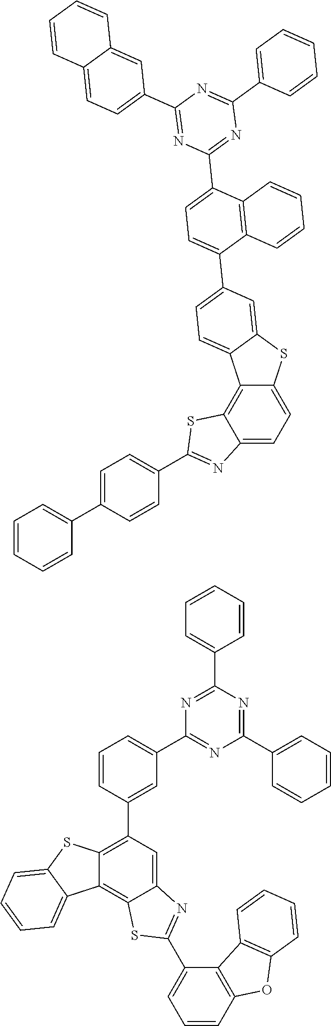 [SMILES: c1ccc(-c2ccc(-c3nc4ccc5sc6cc(-c7ccc(-c8nc(-c9ccccc9)nc(-c9ccc%10ccccc%10c9)n8)c8ccccc78)ccc6c5c4s3)cc2)cc1 ; c1ccc(-c2nc(-c3ccccc3)nc(-c3cccc(-c4cc5nc(-c6cccc7oc8ccccc8c67)sc5c5c4sc4ccccc45)c3)n2)cc1]

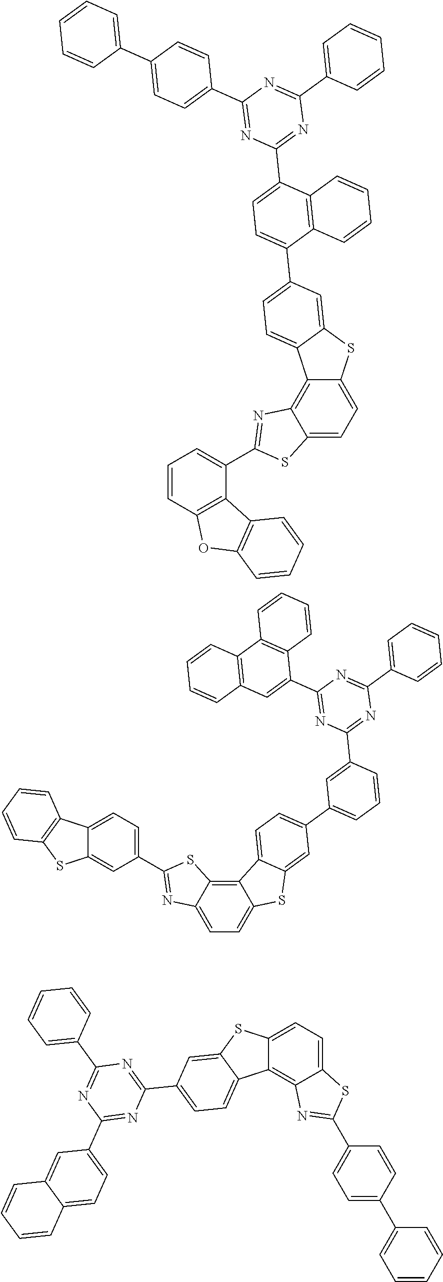

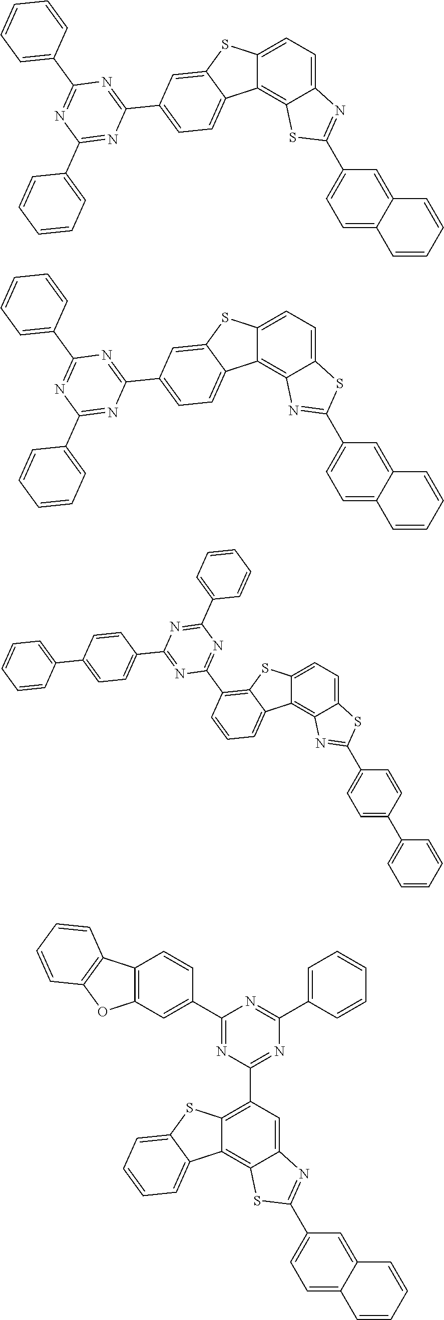

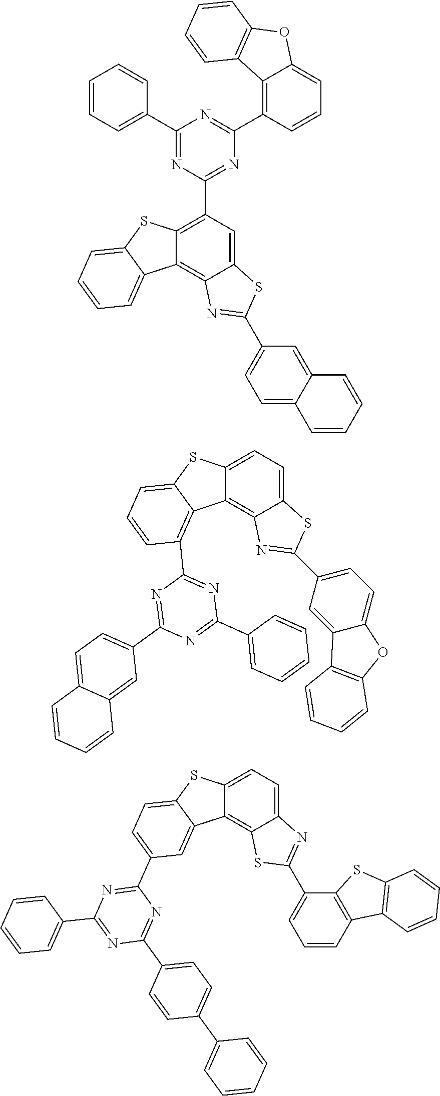

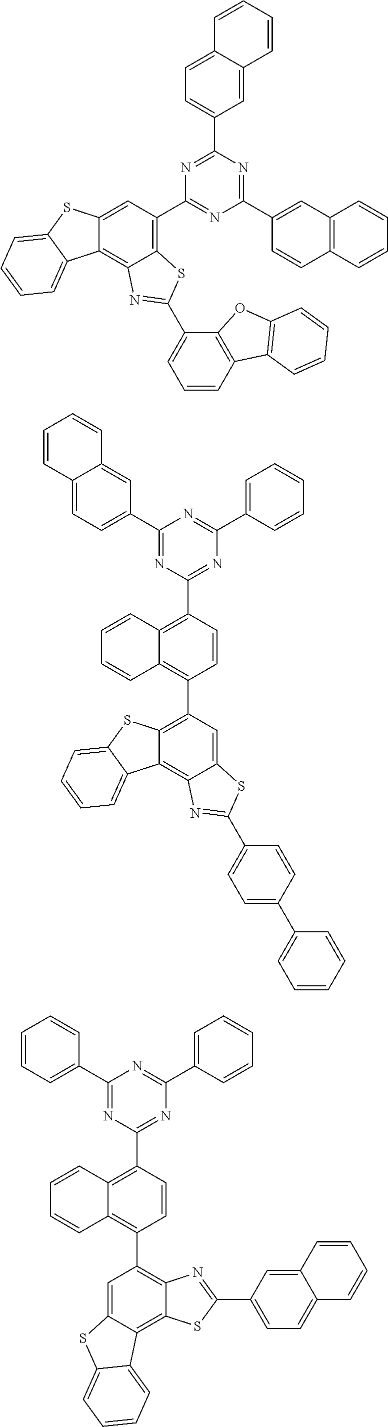 [SMILES: c1ccc2cc(-c3nc(-c4ccc5ccccc5c4)nc(-c4cc5sc6ccccc6c5c5nc(-c6cccc7c6oc6ccccc67)sc45)n3)ccc2c1 ; c1ccc(-c2ccc(-c3nc4c(cc(-c5ccc(-c6nc(-c7ccccc7)nc(-c7ccc8ccccc8c7)n6)c6ccccc56)c5sc6ccccc6c54)s3)cc2)cc1 ; c1ccc(-c2nc(-c3ccccc3)nc(-c3ccc(-c4cc5sc6ccccc6c5c5sc(-c6ccc7ccccc7c6)nc45)c4ccccc34)n2)cc1]

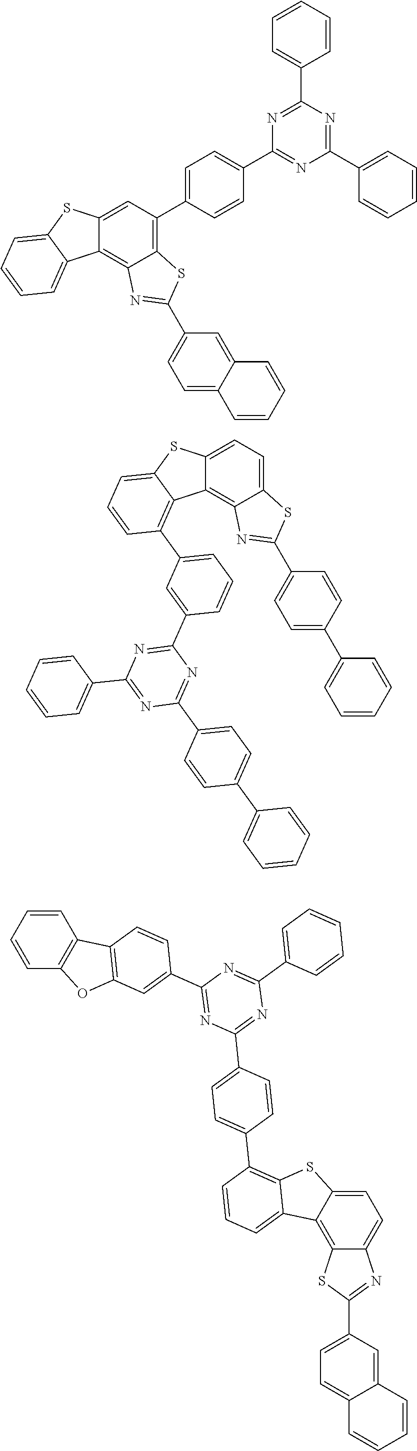

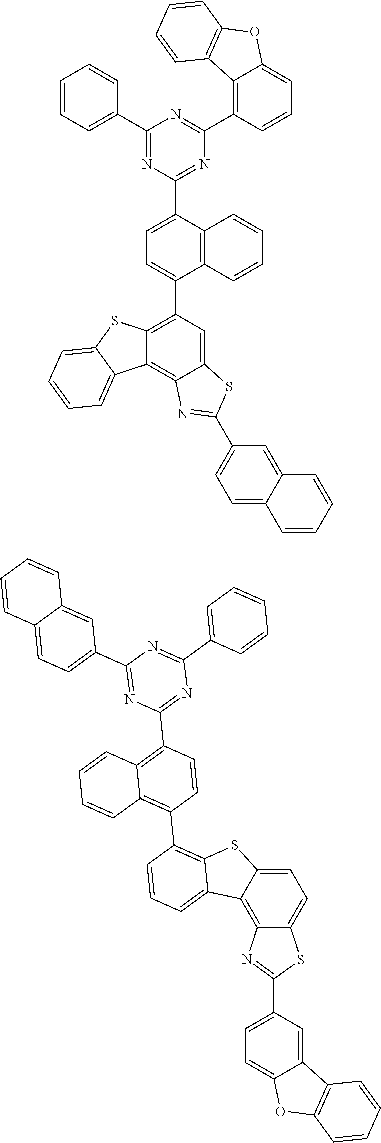 [SMILES: c1ccc(-c2nc(-c3ccc(-c4cc5sc(-c6ccc7ccccc7c6)nc5c5c4sc4ccccc45)c4ccccc34)nc(-c3cccc4oc5ccccc5c34)n2)cc1 ; c1ccc(-c2nc(-c3ccc4ccccc4c3)nc(-c3ccc(-c4cccc5c4sc4ccc6sc(-c7ccc8oc9ccccc9c8c7)nc6c45)c4ccccc34)n2)cc1]

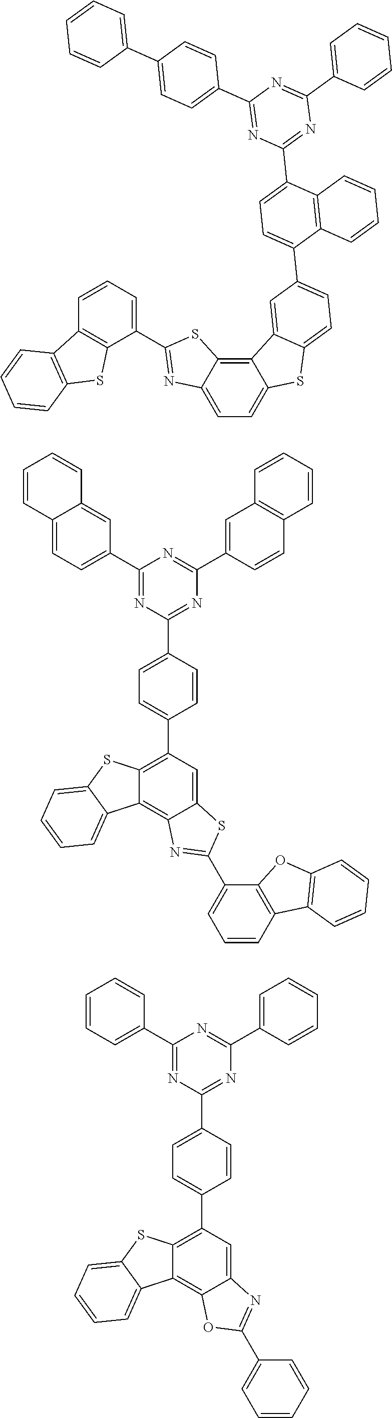

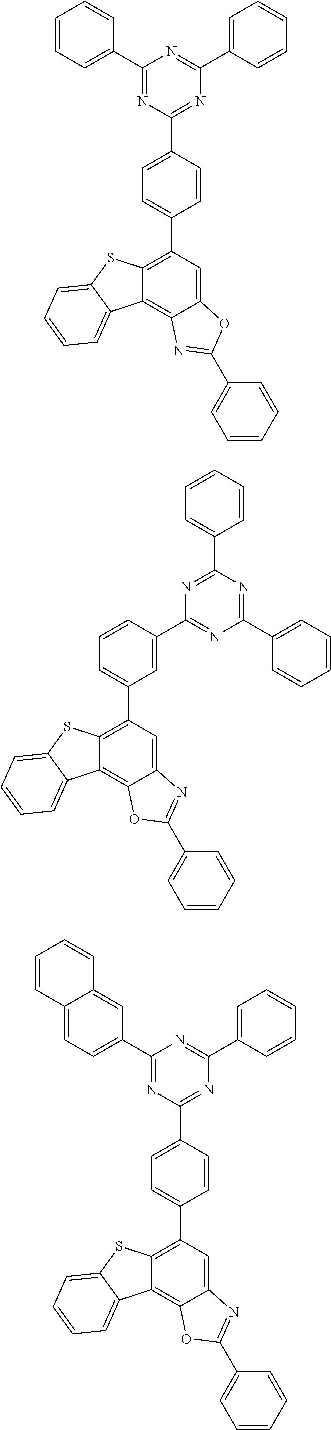

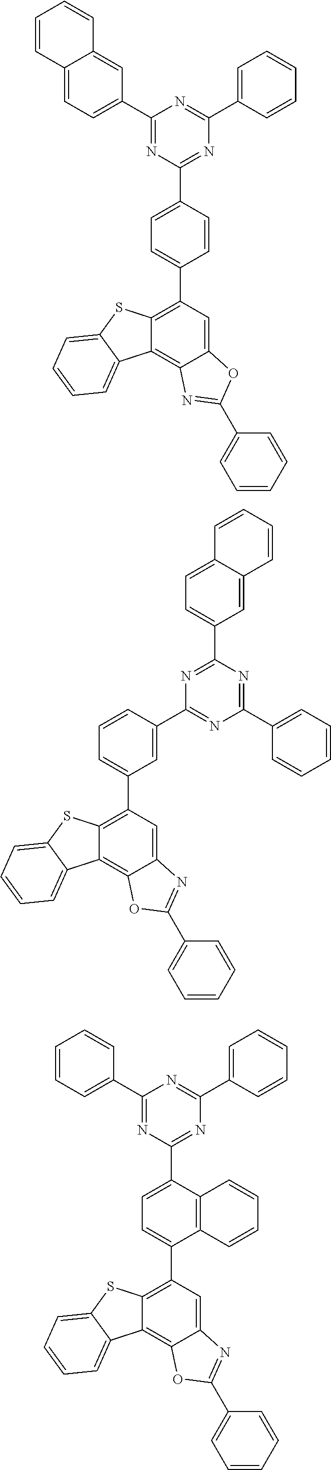

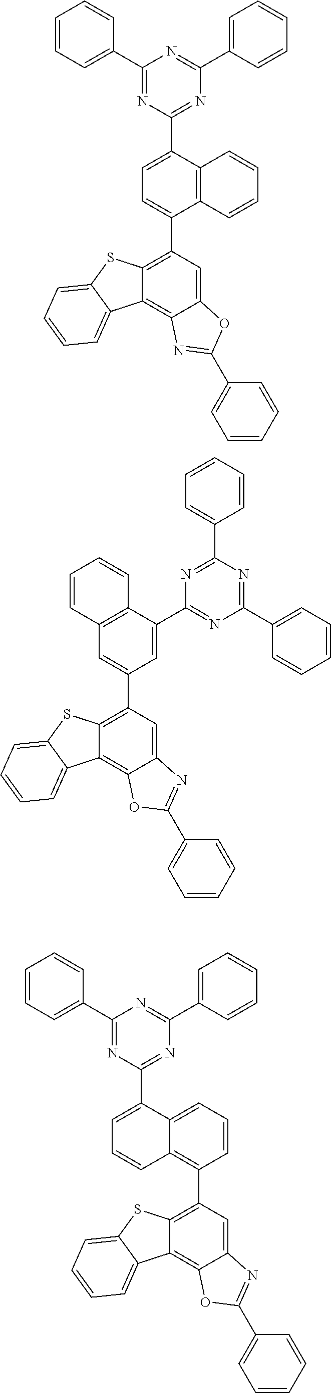

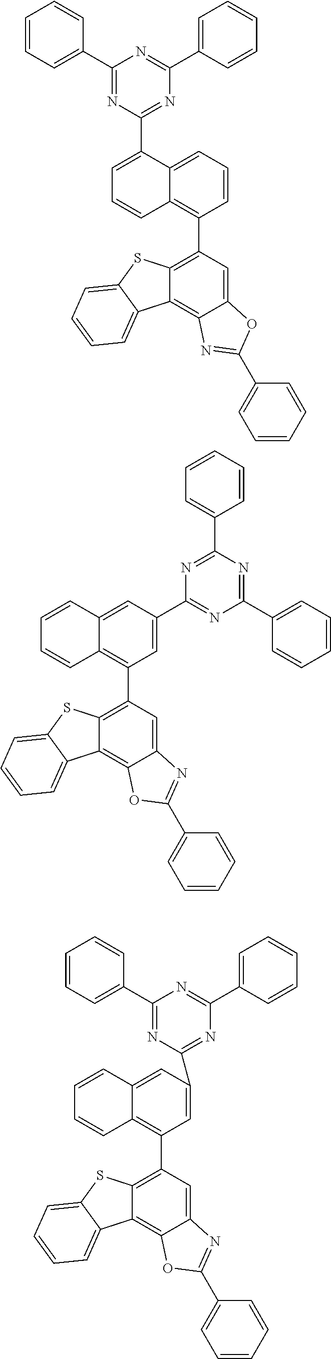

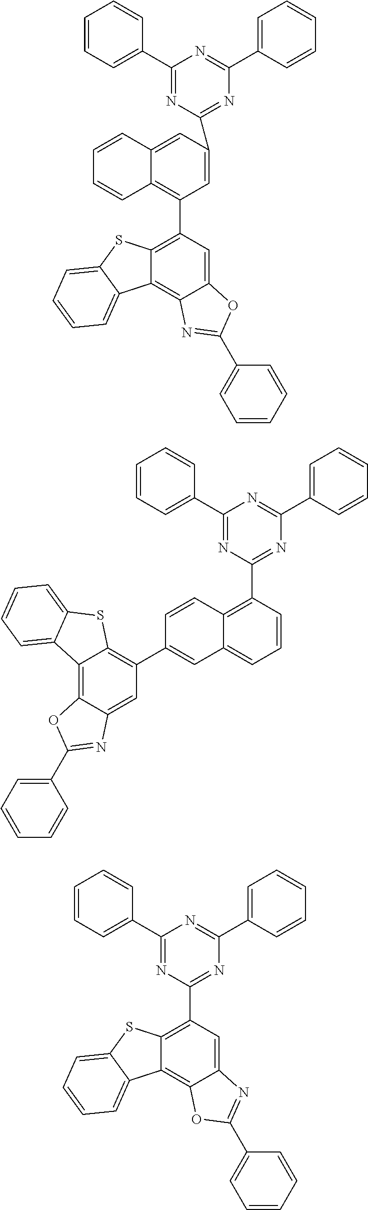 [SMILES: c1ccc(-c2nc(-c3ccccc3)nc(-c3cc(-c4cc5oc(-c6ccccc6)nc5c5c4sc4ccccc45)c4ccccc4c3)n2)cc1 ; c1ccc(-c2nc(-c3ccccc3)nc(-c3cccc4cc(-c5cc6nc(-c7ccccc7)oc6c6c5sc5ccccc56)ccc34)n2)cc1 ; c1ccc(-c2nc(-c3ccccc3)nc(-c3cc4nc(-c5ccccc5)oc4c4c3sc3ccccc34)n2)cc1]

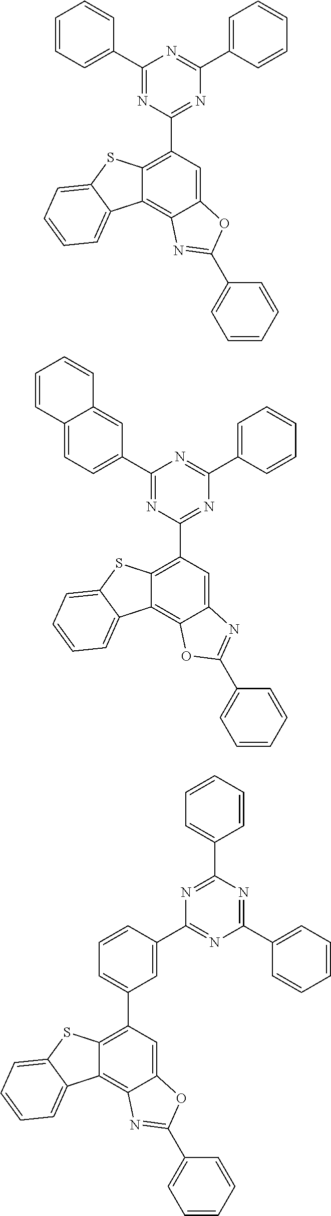

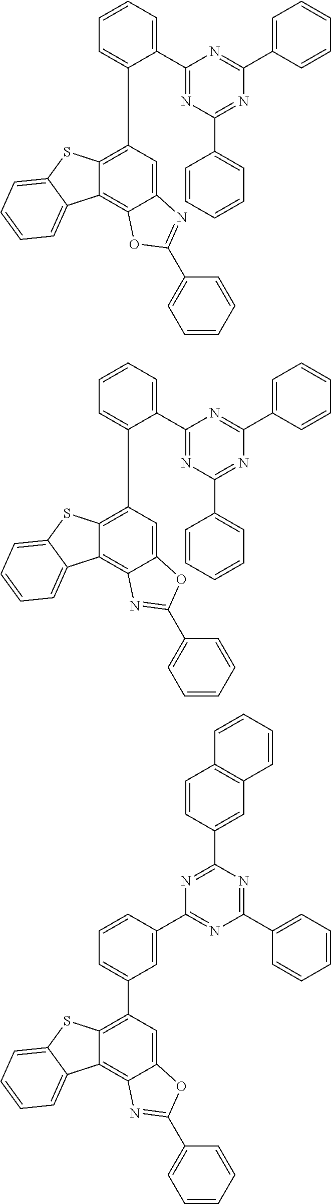

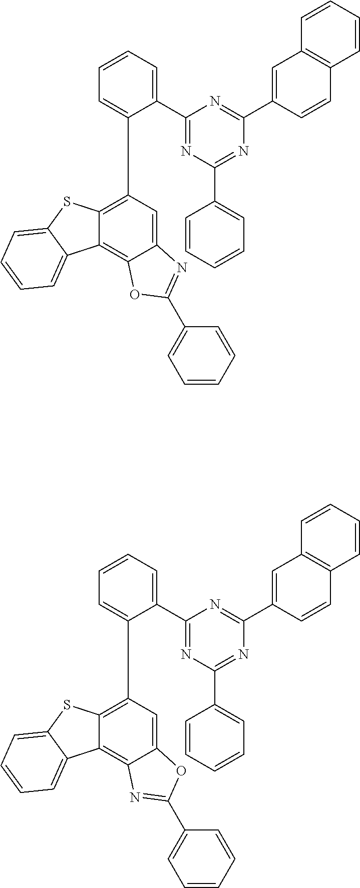 [SMILES: c1ccc(-c2nc(-c3ccc4ccccc4c3)nc(-c3ccccc3-c3cc4nc(-c5ccccc5)oc4c4c3sc3ccccc34)n2)cc1 ; c1ccc(-c2nc(-c3ccc4ccccc4c3)nc(-c3ccccc3-c3cc4oc(-c5ccccc5)nc4c4c3sc3ccccc34)n2)cc1]

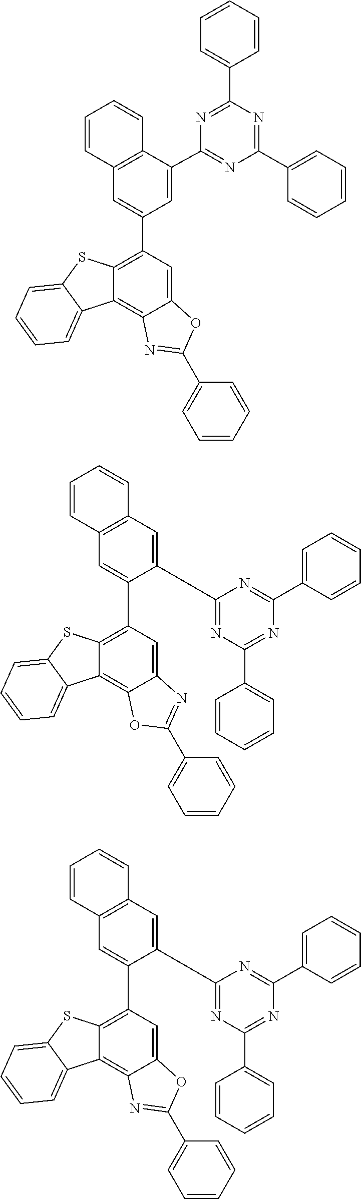 [SMILES: c1ccc(-c2nc(-c3ccccc3)nc(-c3cc(-c4cc5oc(-c6ccccc6)nc5c5c4sc4ccccc45)cc4ccccc34)n2)cc1 ; c1ccc(-c2nc(-c3ccccc3)nc(-c3cc4ccccc4cc3-c3cc4nc(-c5ccccc5)oc4c4c3sc3ccccc34)n2)cc1 ; c1ccc(-c2nc(-c3ccccc3)nc(-c3cc4ccccc4cc3-c3cc4oc(-c5ccccc5)nc4c4c3sc3ccccc34)n2)cc1]

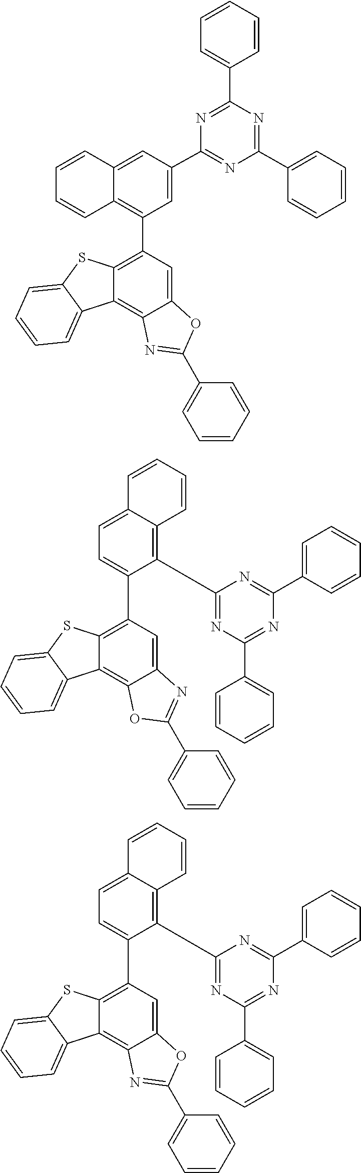

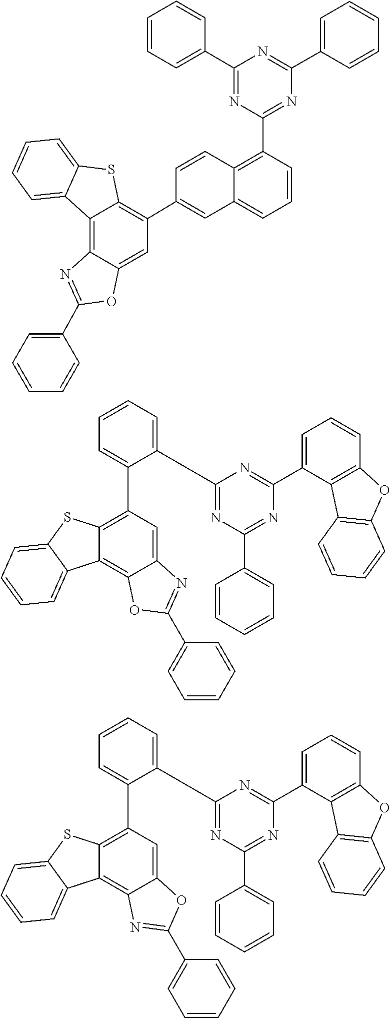

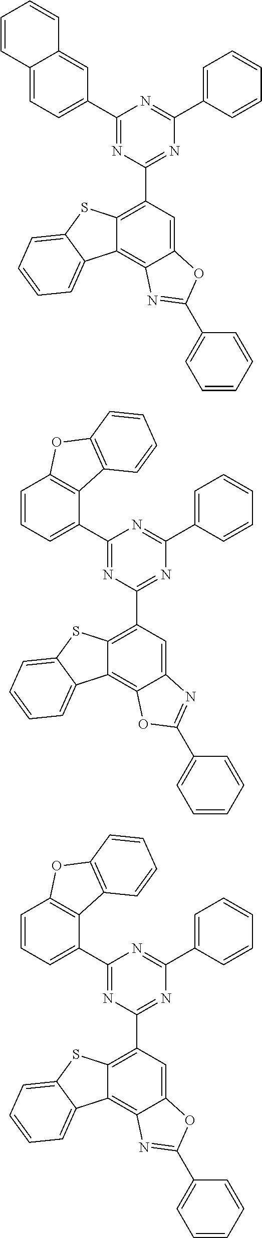 [SMILES: c1ccc(-c2nc(-c3ccc4ccccc4c3)nc(-c3cc4oc(-c5ccccc5)nc4c4c3sc3ccccc34)n2)cc1 ; c1ccc(-c2nc(-c3cc4nc(-c5ccccc5)oc4c4c3sc3ccccc34)nc(-c3cccc4oc5ccccc5c34)n2)cc1 ; c1ccc(-c2nc(-c3cc4oc(-c5ccccc5)nc4c4c3sc3ccccc34)nc(-c3cccc4oc5ccccc5c34)n2)cc1]

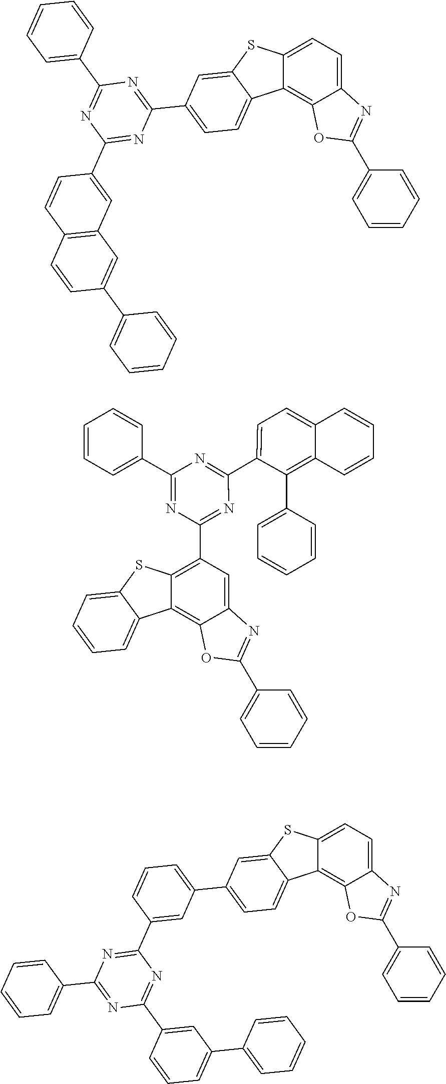

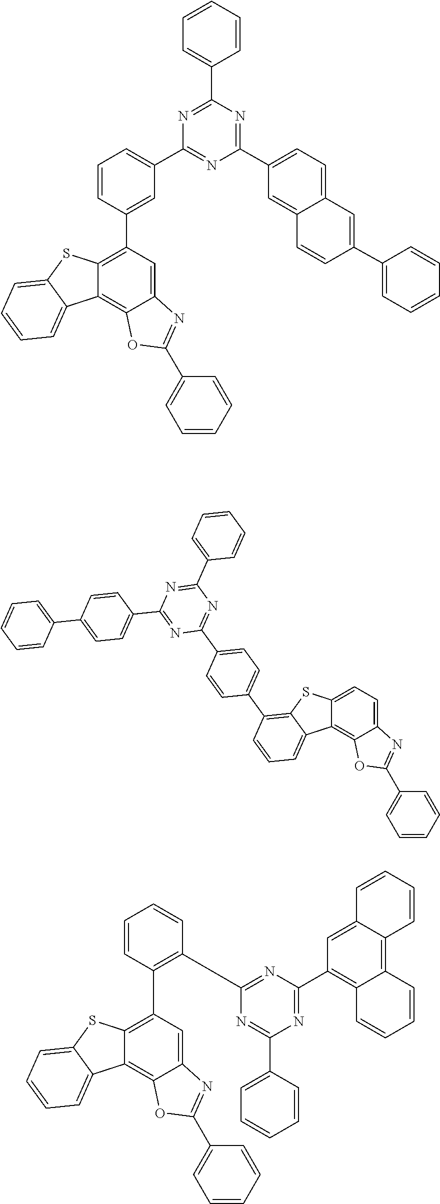

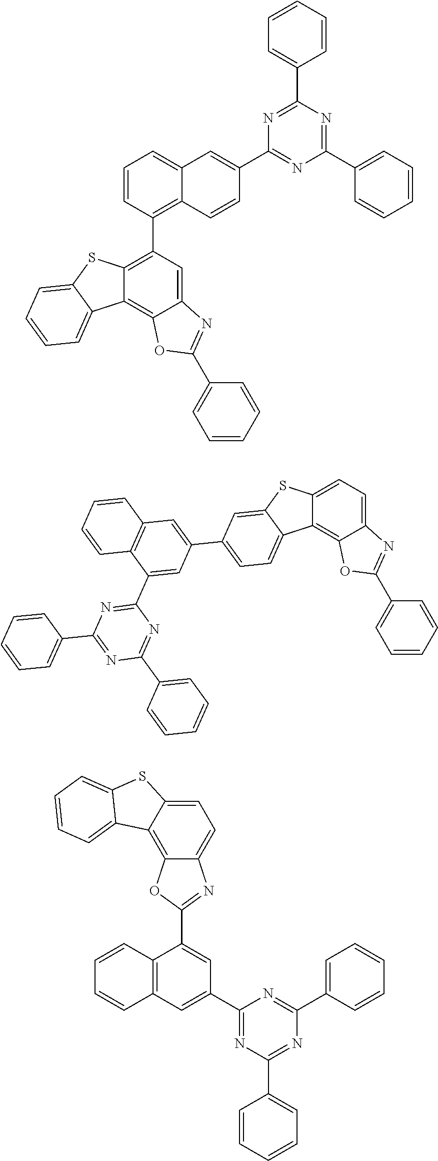 [SMILES: c1ccc(-c2nc(-c3ccccc3)nc(-c3ccc4c(-c5cc6nc(-c7ccccc7)oc6c6c5sc5ccccc56)cccc4c3)n2)cc1 ; c1ccc(-c2nc(-c3ccccc3)nc(-c3cc(-c4ccc5c(c4)sc4ccc6nc(-c7ccccc7)oc6c45)cc4ccccc34)n2)cc1 ; c1ccc(-c2nc(-c3ccccc3)nc(-c3cc(-c4nc5ccc6sc7ccccc7c6c5o4)c4ccccc4c3)n2)cc1]

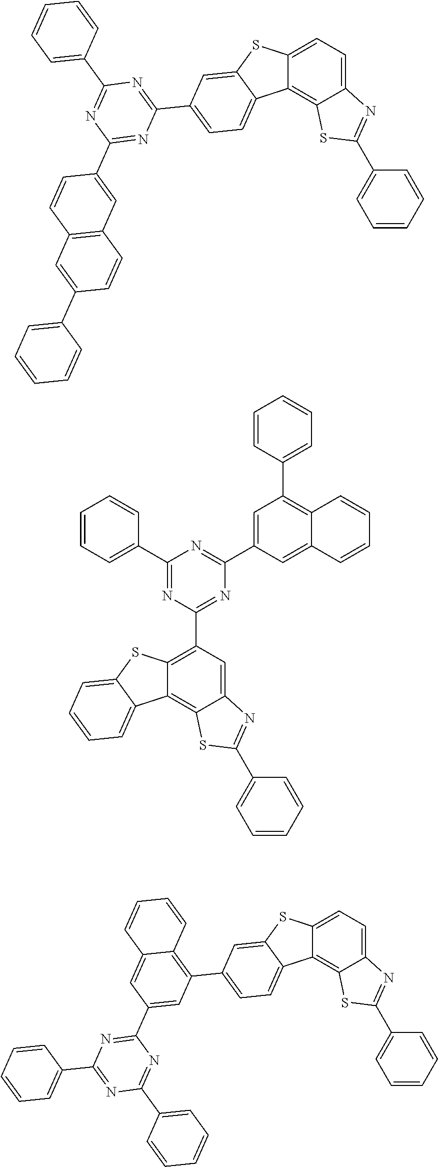 [SMILES: c1ccc(-c2ccc3cc(-c4nc(-c5ccccc5)nc(-c5ccc6c(c5)sc5ccc7nc(-c8ccccc8)sc7c56)n4)ccc3c2)cc1 ; c1ccc(-c2nc(-c3cc(-c4ccccc4)c4ccccc4c3)nc(-c3cc4nc(-c5ccccc5)sc4c4c3sc3ccccc34)n2)cc1 ; c1ccc(-c2nc(-c3ccccc3)nc(-c3cc(-c4ccc5c(c4)sc4ccc6nc(-c7ccccc7)sc6c45)c4ccccc4c3)n2)cc1]

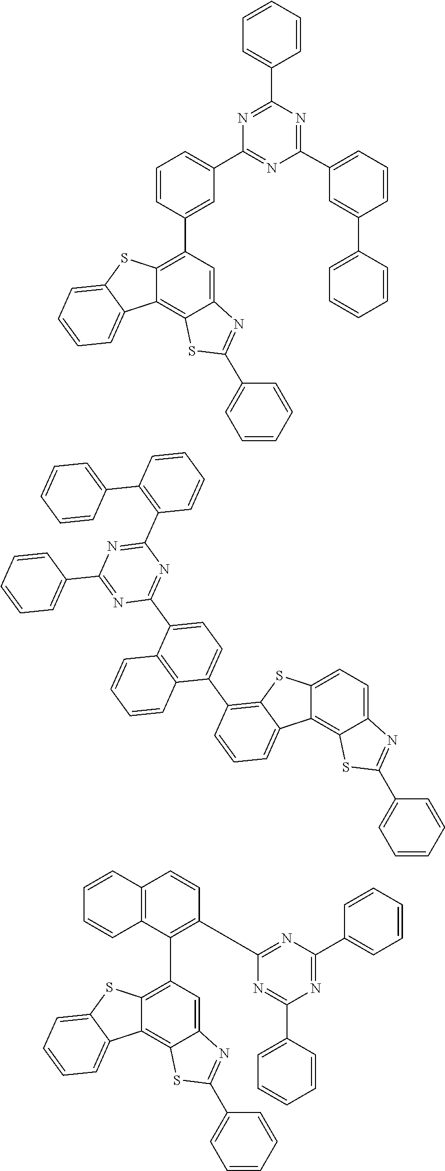

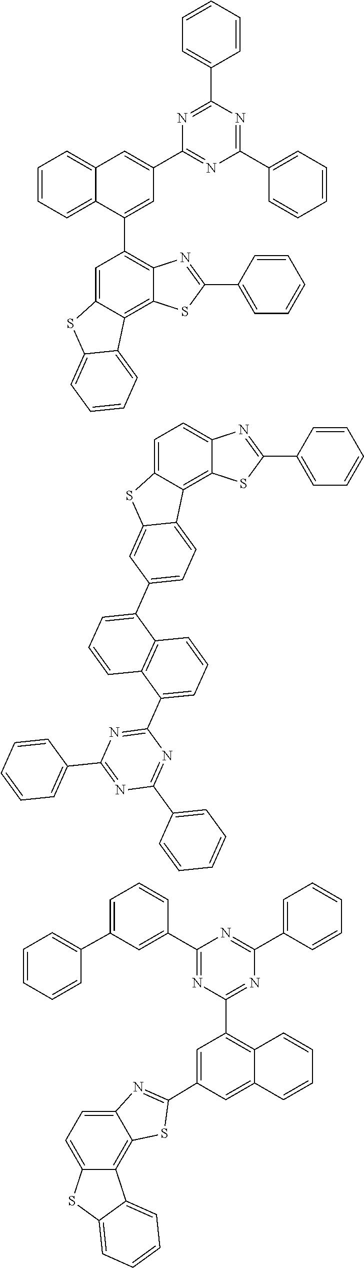

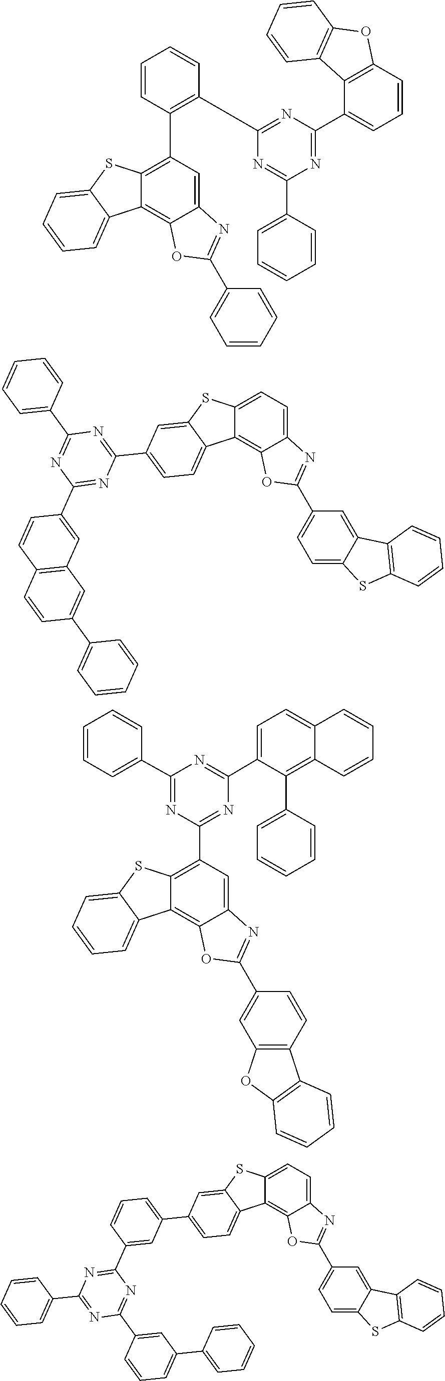

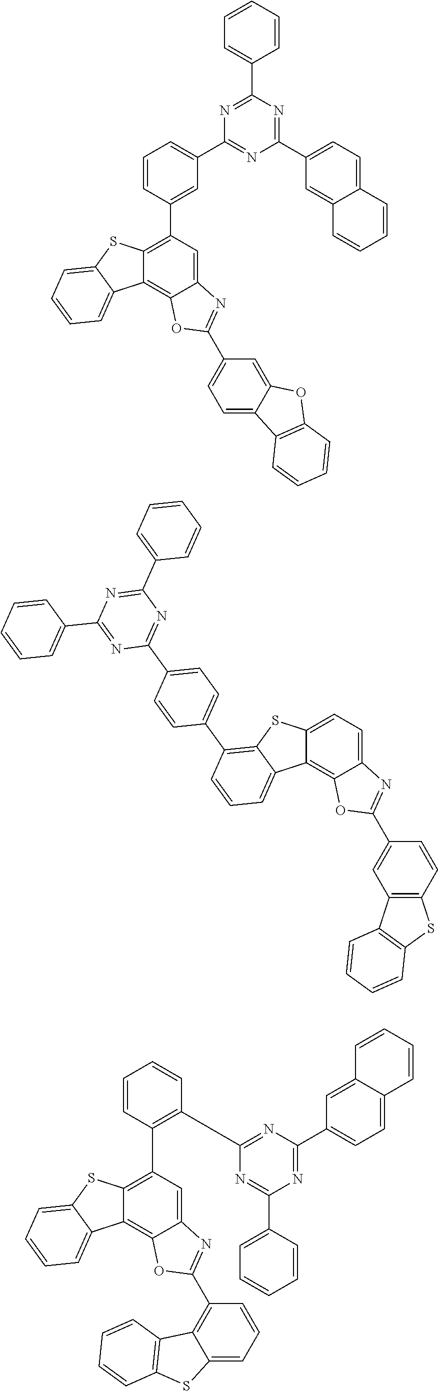

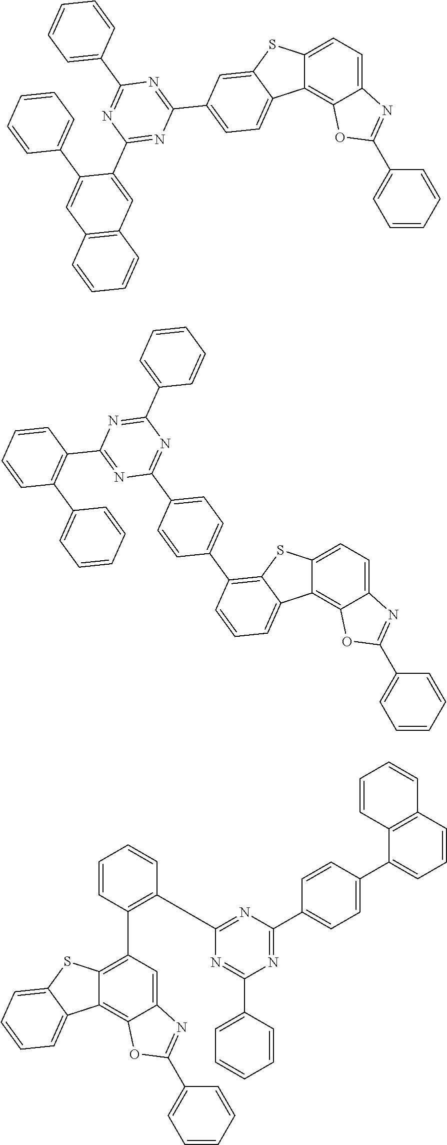

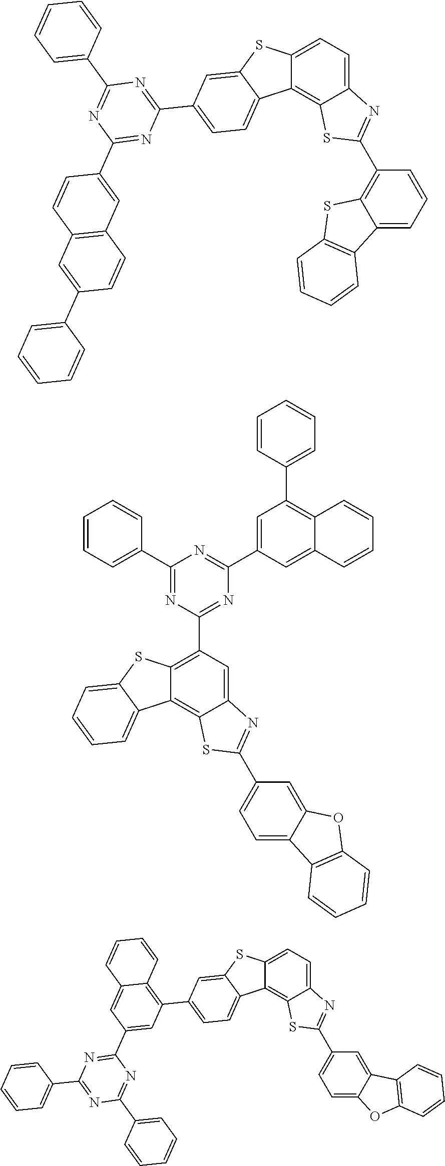 [SMILES: c1ccc(-c2ccc3cc(-c4nc(-c5ccccc5)nc(-c5ccc6c(c5)sc5ccc7nc(-c8cccc9c8sc8ccccc89)sc7c56)n4)ccc3c2)cc1 ; c1ccc(-c2nc(-c3cc(-c4ccccc4)c4ccccc4c3)nc(-c3cc4nc(-c5ccc6c(c5)oc5ccccc56)sc4c4c3sc3ccccc34)n2)cc1 ; c1ccc(-c2nc(-c3ccccc3)nc(-c3cc(-c4ccc5c(c4)sc4ccc6nc(-c7ccc8oc9ccccc9c8c7)sc6c45)c4ccccc4c3)n2)cc1]

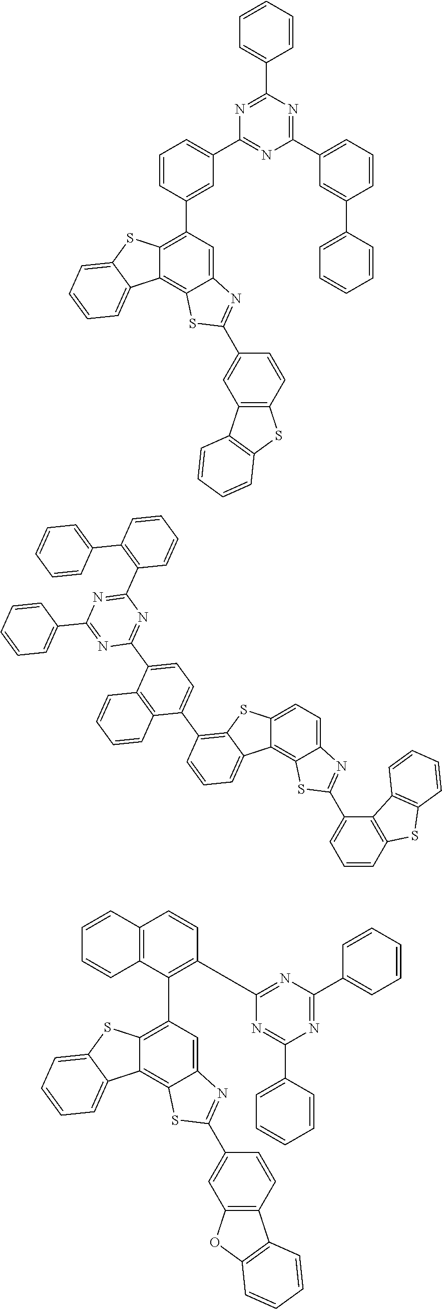 [SMILES: c1ccc(-c2cccc(-c3nc(-c4ccccc4)nc(-c4cccc(-c5cc6nc(-c7ccc8sc9ccccc9c8c7)sc6c6c5sc5ccccc56)c4)n3)c2)cc1 ; c1ccc(-c2nc(-c3ccccc3-c3ccccc3)nc(-c3ccc(-c4cccc5c4sc4ccc6nc(-c7cccc8sc9ccccc9c78)sc6c45)c4ccccc34)n2)cc1 ; c1ccc(-c2nc(-c3ccccc3)nc(-c3ccc4ccccc4c3-c3cc4nc(-c5ccc6c(c5)oc5ccccc56)sc4c4c3sc3ccccc34)n2)cc1]

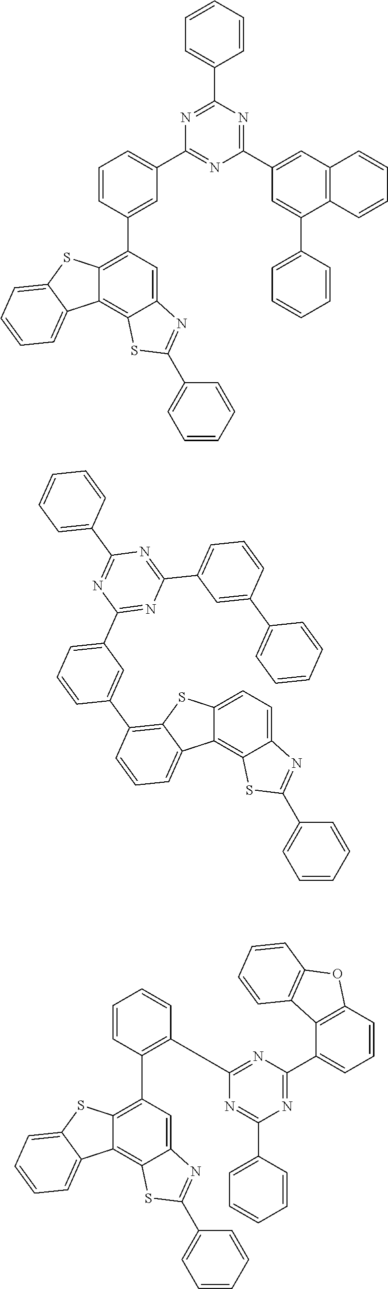 [SMILES: c1ccc(-c2nc(-c3cccc(-c4cc5nc(-c6ccccc6)sc5c5c4sc4ccccc45)c3)nc(-c3cc(-c4ccccc4)c4ccccc4c3)n2)cc1 ; c1ccc(-c2cccc(-c3nc(-c4ccccc4)nc(-c4cccc(-c5cccc6c5sc5ccc7nc(-c8ccccc8)sc7c56)c4)n3)c2)cc1 ; c1ccc(-c2nc(-c3ccccc3-c3cc4nc(-c5ccccc5)sc4c4c3sc3ccccc34)nc(-c3cccc4oc5ccccc5c34)n2)cc1]

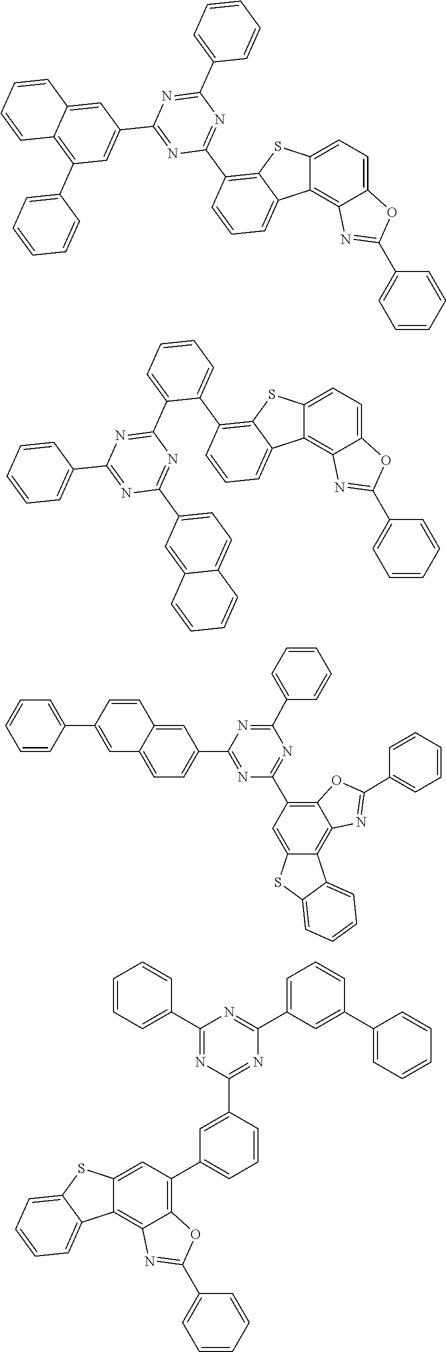

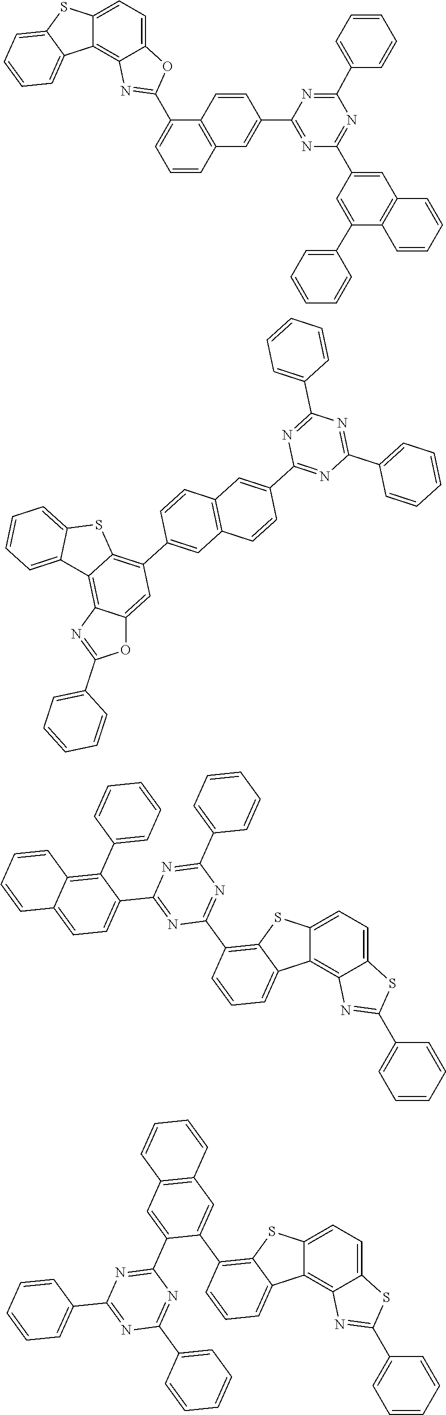

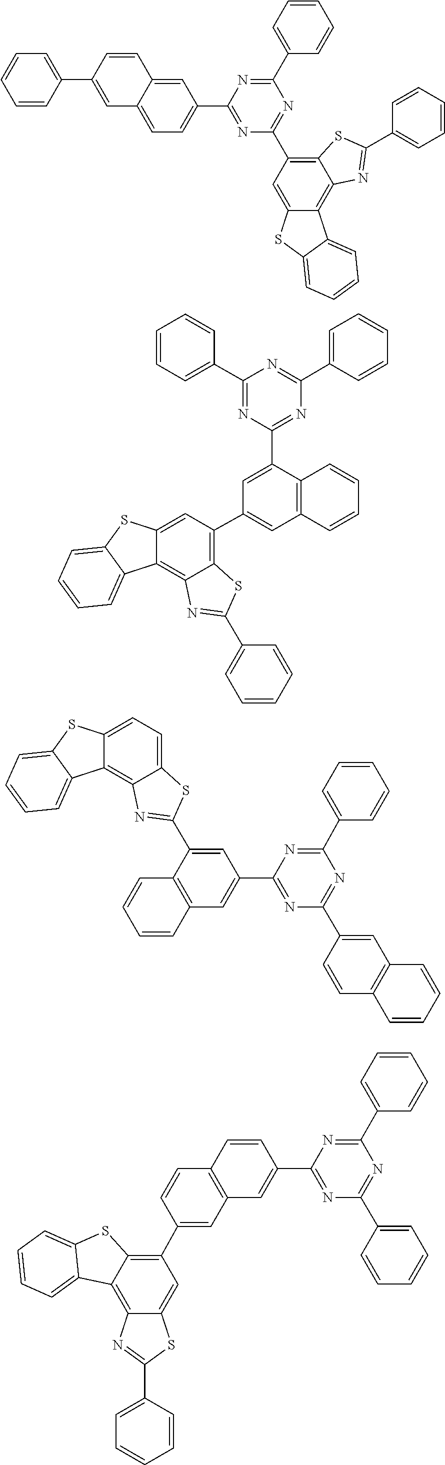

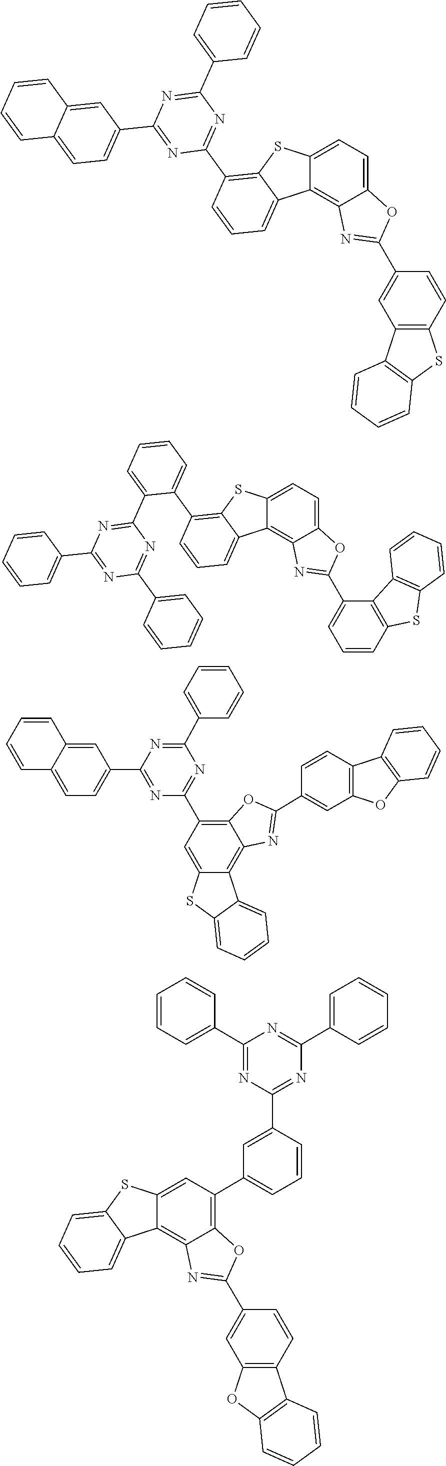

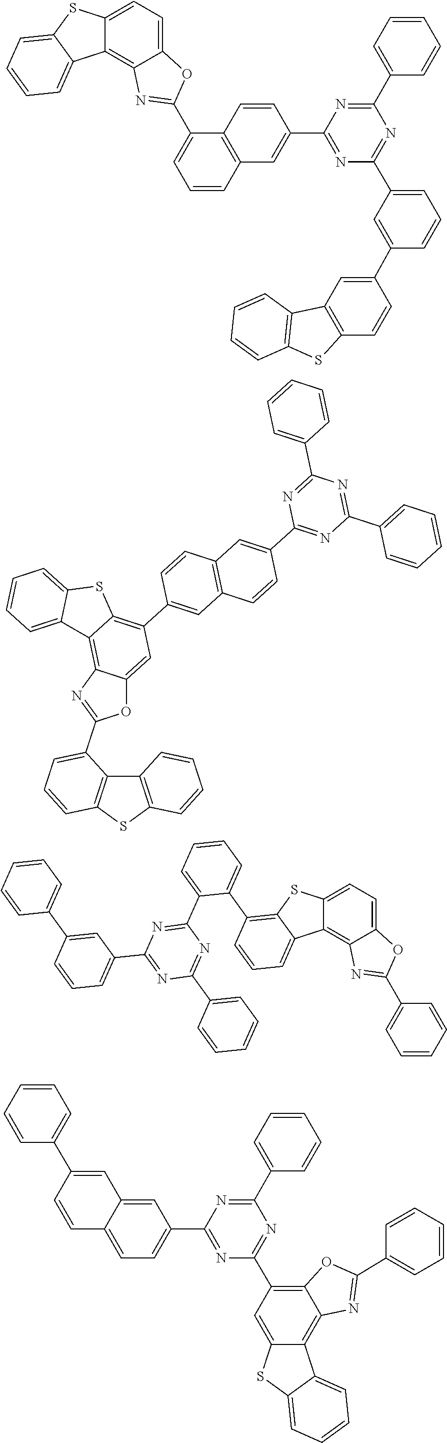

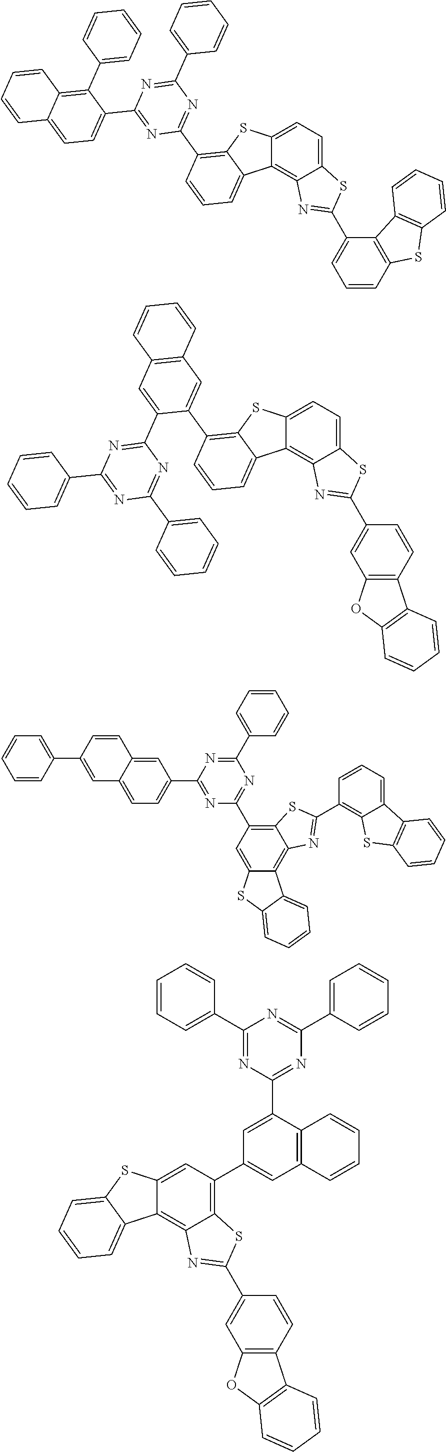 [SMILES: c1ccc(-c2nc(-c3ccc4ccccc4c3-c3ccccc3)nc(-c3cccc4c3sc3ccc5sc(-c6cccc7sc8ccccc8c67)nc5c34)n2)cc1 ; c1ccc(-c2nc(-c3ccccc3)nc(-c3cc4ccccc4cc3-c3cccc4c3sc3ccc5sc(-c6ccc7c(c6)oc6ccccc67)nc5c34)n2)cc1 ; c1ccc(-c2ccc3cc(-c4nc(-c5ccccc5)nc(-c5cc6sc7ccccc7c6c6nc(-c7cccc8c7sc7ccccc78)sc56)n4)ccc3c2)cc1 ; c1ccc(-c2nc(-c3ccccc3)nc(-c3cc(-c4cc5sc6ccccc6c5c5nc(-c6ccc7c(c6)oc6ccccc67)sc45)cc4ccccc34)n2)cc1]

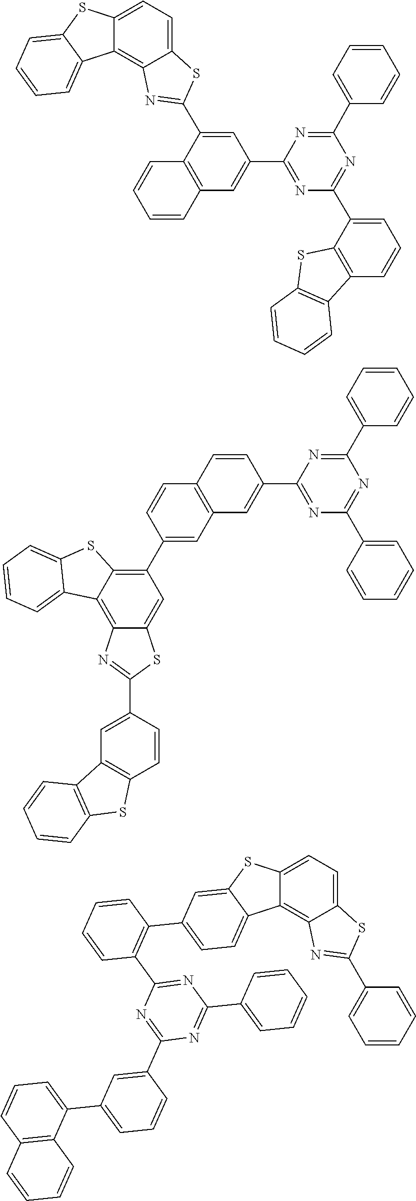

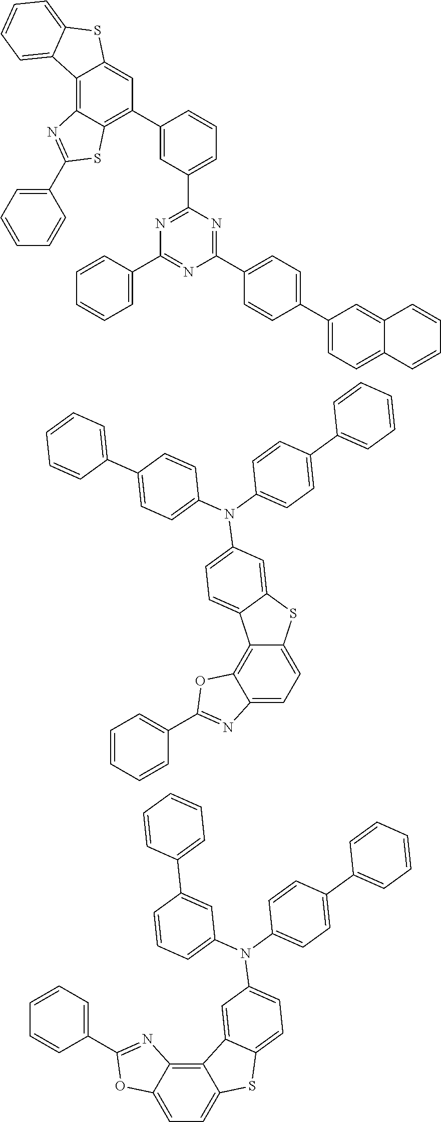

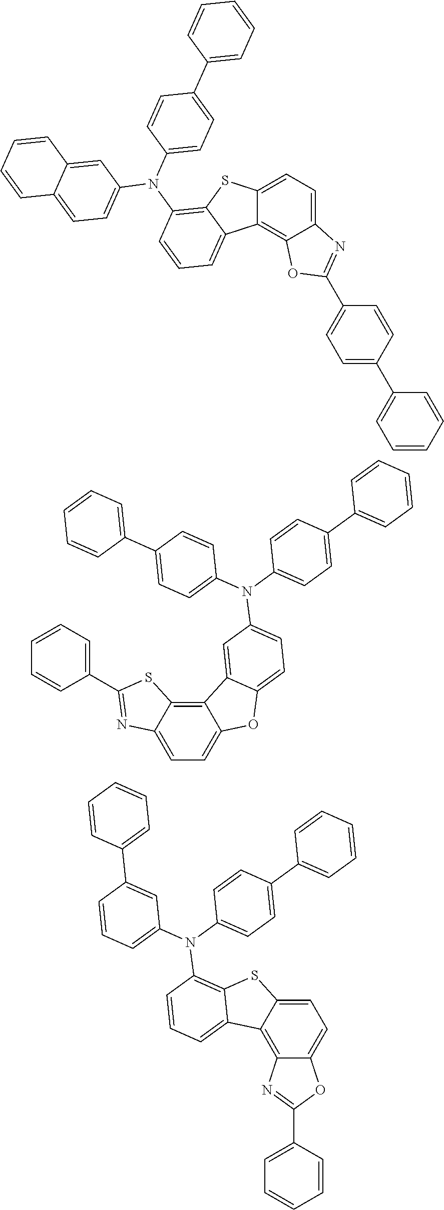

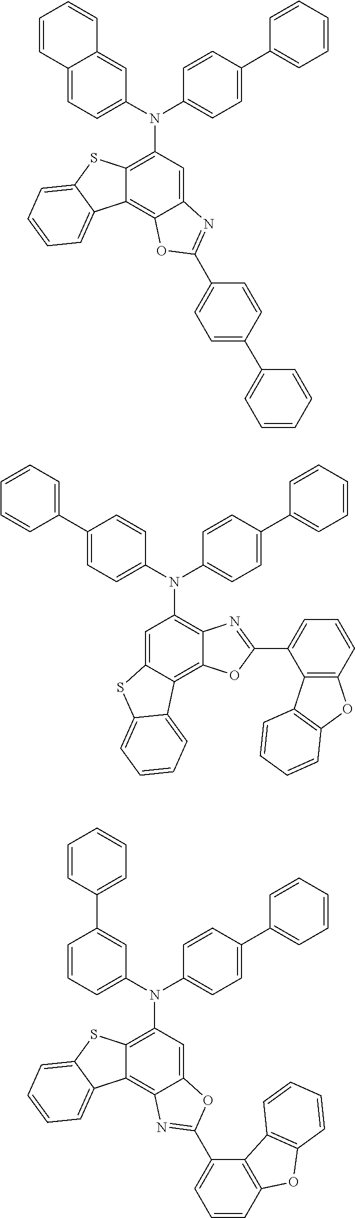

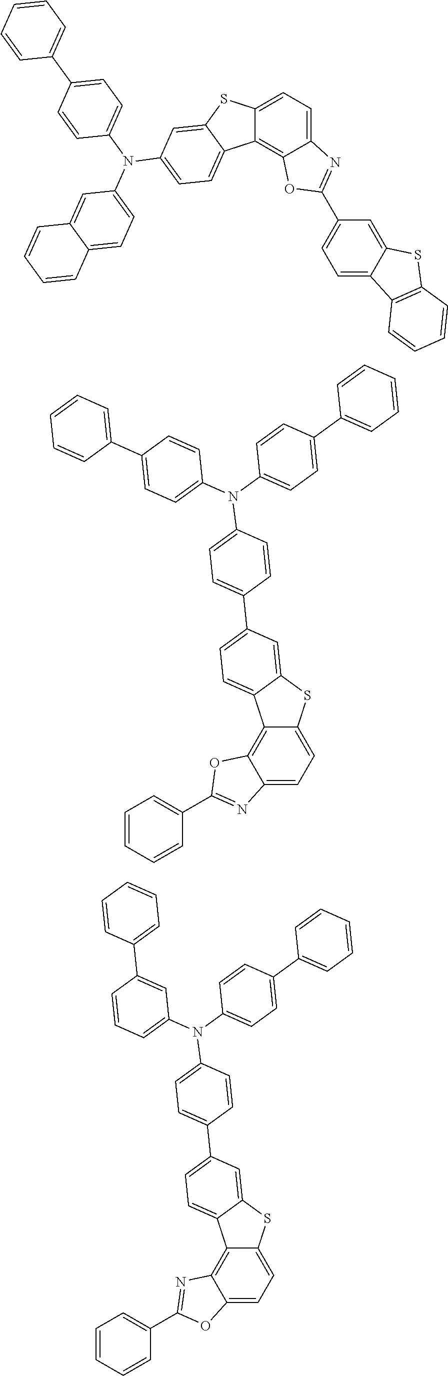

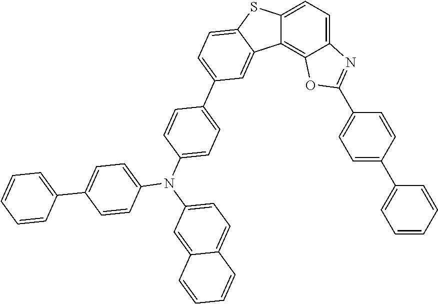 [SMILES: c1ccc(-c2ccc(-c3nc4ccc5sc6ccc(-c7ccc(N(c8ccc(-c9ccccc9)cc8)c8ccc9ccccc9c8)cc7)cc6c5c4o3)cc2)cc1]

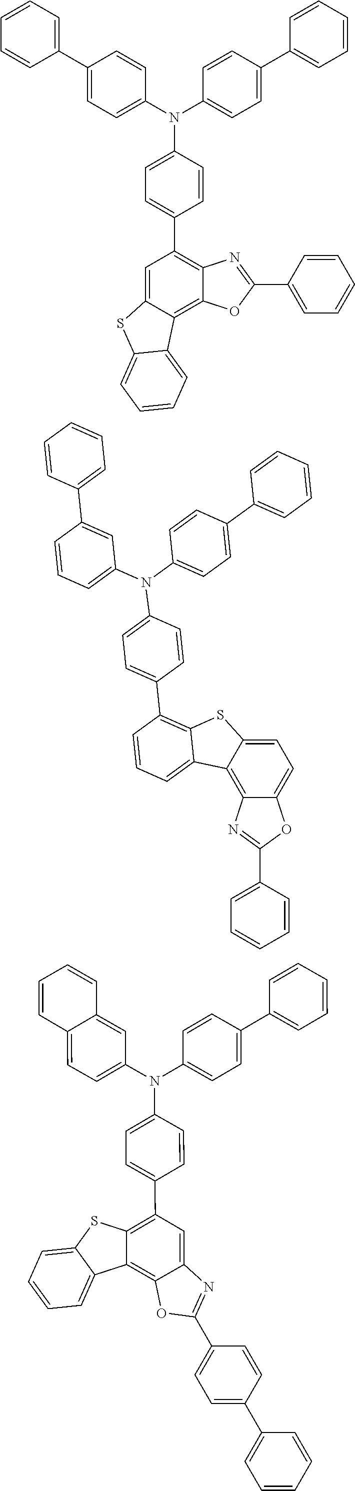

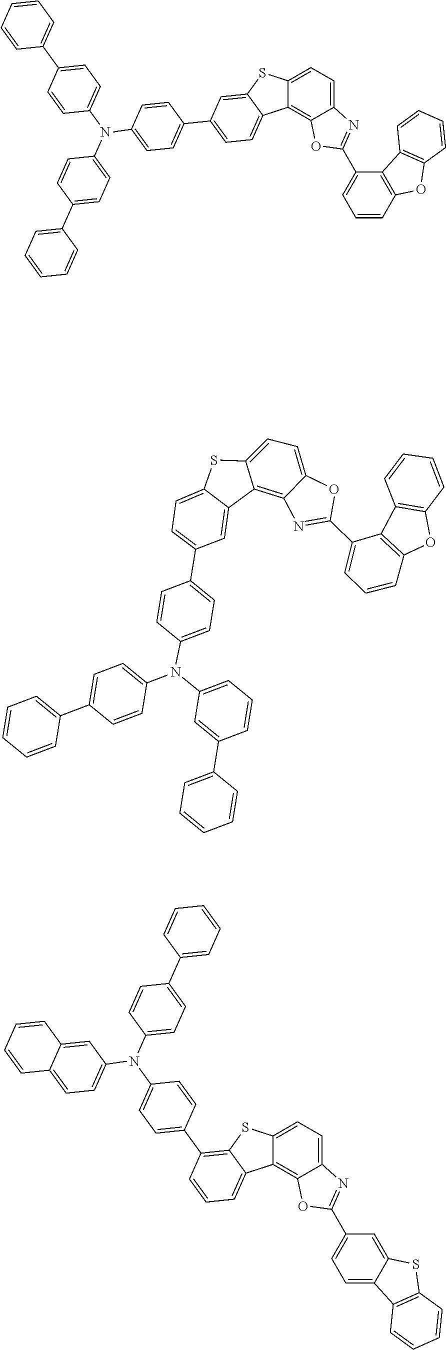 [SMILES: c1ccc(-c2ccc(N(c3ccc(-c4ccccc4)cc3)c3ccc(-c4ccc5c(c4)sc4ccc6nc(-c7cccc8oc9ccccc9c78)oc6c45)cc3)cc2)cc1 ; c1ccc(-c2ccc(N(c3ccc(-c4ccc5sc6ccc7oc(-c8cccc9oc%10ccccc%10c89)nc7c6c5c4)cc3)c3cccc(-c4ccccc4)c3)cc2)cc1 ; c1ccc(-c2ccc(N(c3ccc(-c4cccc5c4sc4ccc6nc(-c7ccc8c(c7)sc7ccccc78)oc6c45)cc3)c3ccc4ccccc4c3)cc2)cc1]

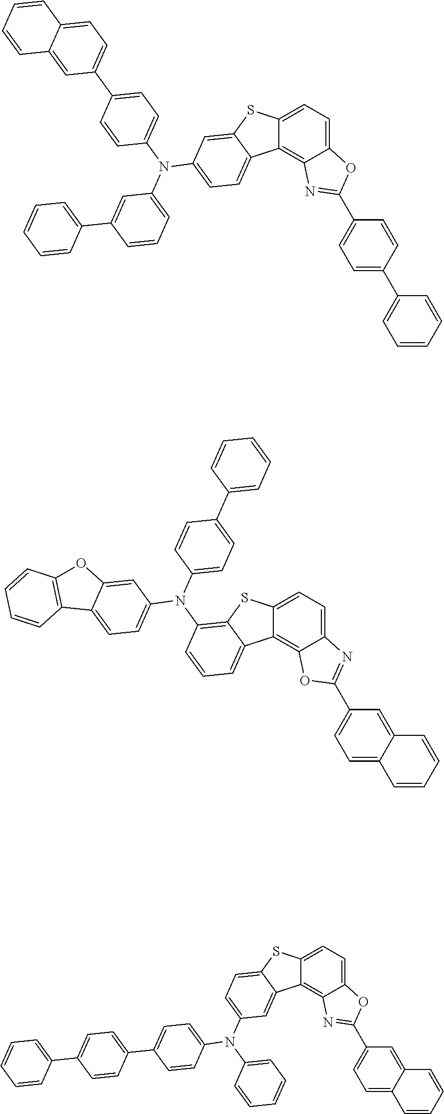 [SMILES: c1ccc(-c2ccc(-c3nc4c(ccc5sc6cc(N(c7ccc(-c8ccc9ccccc9c8)cc7)c7cccc(-c8ccccc8)c7)ccc6c54)o3)cc2)cc1 ; c1ccc(-c2ccc(N(c3ccc4c(c3)oc3ccccc34)c3cccc4c3sc3ccc5nc(-c6ccc7ccccc7c6)oc5c34)cc2)cc1 ; c1ccc(-c2ccc(-c3ccc(N(c4ccccc4)c4ccc5sc6ccc7oc(-c8ccc9ccccc9c8)nc7c6c5c4)cc3)cc2)cc1]

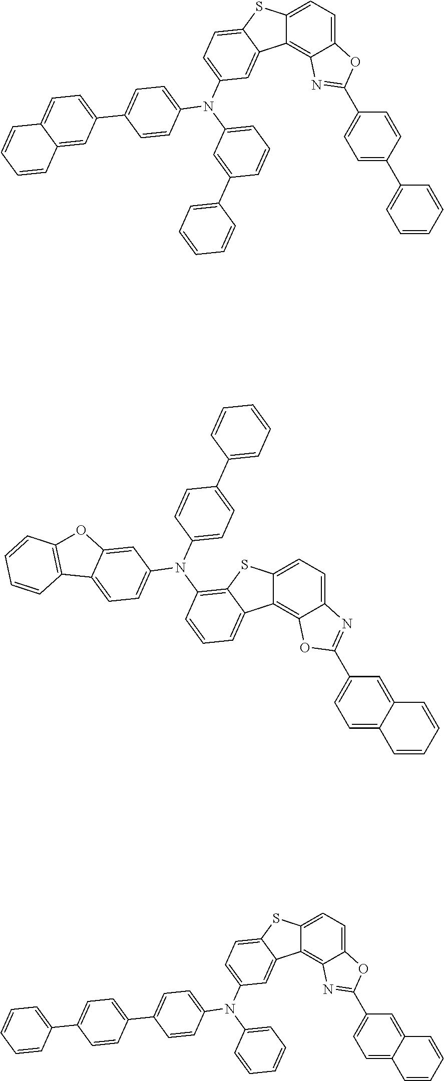 [SMILES: c1ccc(-c2ccc(-c3nc4c(ccc5sc6ccc(N(c7ccc(-c8ccc9ccccc9c8)cc7)c7cccc(-c8ccccc8)c7)cc6c54)o3)cc2)cc1 ; c1ccc(-c2ccc(N(c3ccc4c(c3)oc3ccccc34)c3cccc4c3sc3ccc5nc(-c6ccc7ccccc7c6)oc5c34)cc2)cc1 ; c1ccc(-c2ccc(-c3ccc(N(c4ccccc4)c4ccc5sc6ccc7oc(-c8ccc9ccccc9c8)nc7c6c5c4)cc3)cc2)cc1]

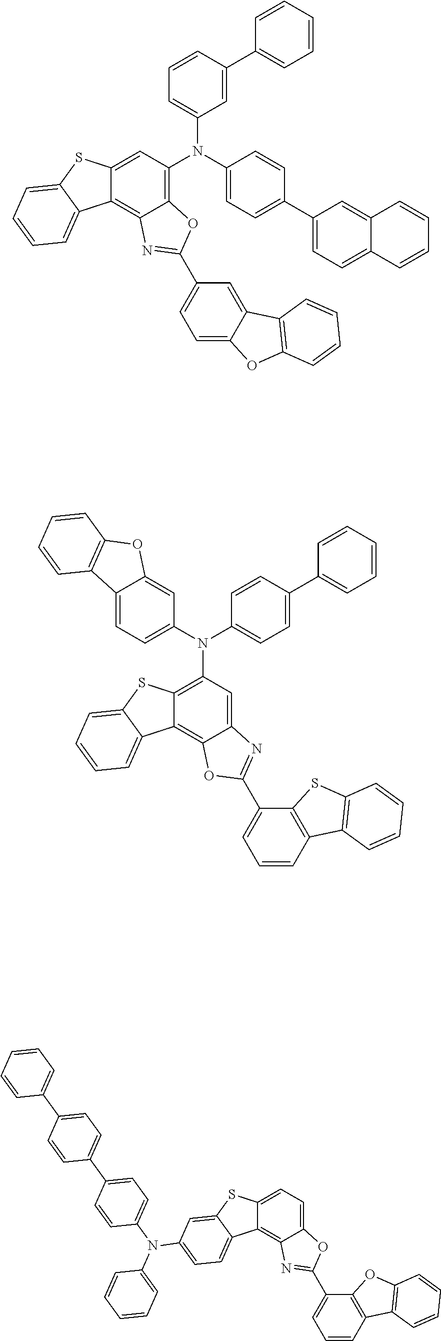 [SMILES: c1ccc(-c2cccc(N(c3ccc(-c4ccc5ccccc5c4)cc3)c3cc4sc5ccccc5c4c4nc(-c5ccc6oc7ccccc7c6c5)oc34)c2)cc1 ; c1ccc(-c2ccc(N(c3ccc4c(c3)oc3ccccc34)c3cc4nc(-c5cccc6c5sc5ccccc56)oc4c4c3sc3ccccc34)cc2)cc1 ; c1ccc(-c2ccc(-c3ccc(N(c4ccccc4)c4ccc5c(c4)sc4ccc6oc(-c7cccc8c7oc7ccccc78)nc6c45)cc3)cc2)cc1]

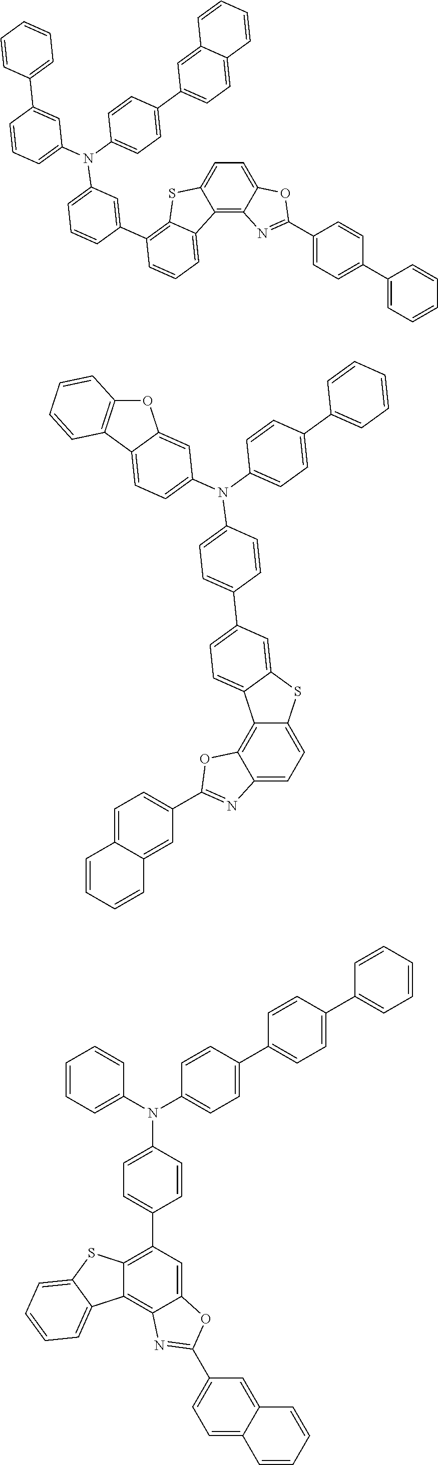 [SMILES: c1ccc(-c2ccc(-c3nc4c(ccc5sc6c(-c7cccc(N(c8ccc(-c9ccc%10ccccc%10c9)cc8)c8cccc(-c9ccccc9)c8)c7)cccc6c54)o3)cc2)cc1 ; c1ccc(-c2ccc(N(c3ccc(-c4ccc5c(c4)sc4ccc6nc(-c7ccc8ccccc8c7)oc6c45)cc3)c3ccc4c(c3)oc3ccccc34)cc2)cc1 ; c1ccc(-c2ccc(-c3ccc(N(c4ccccc4)c4ccc(-c5cc6oc(-c7ccc8ccccc8c7)nc6c6c5sc5ccccc56)cc4)cc3)cc2)cc1]

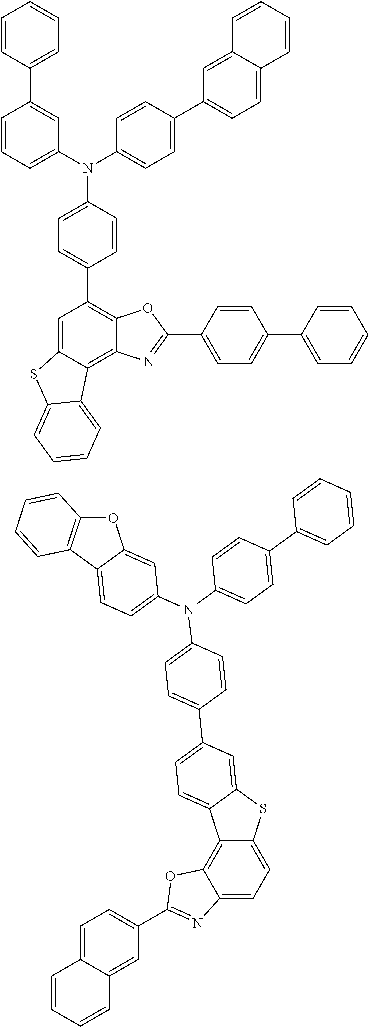

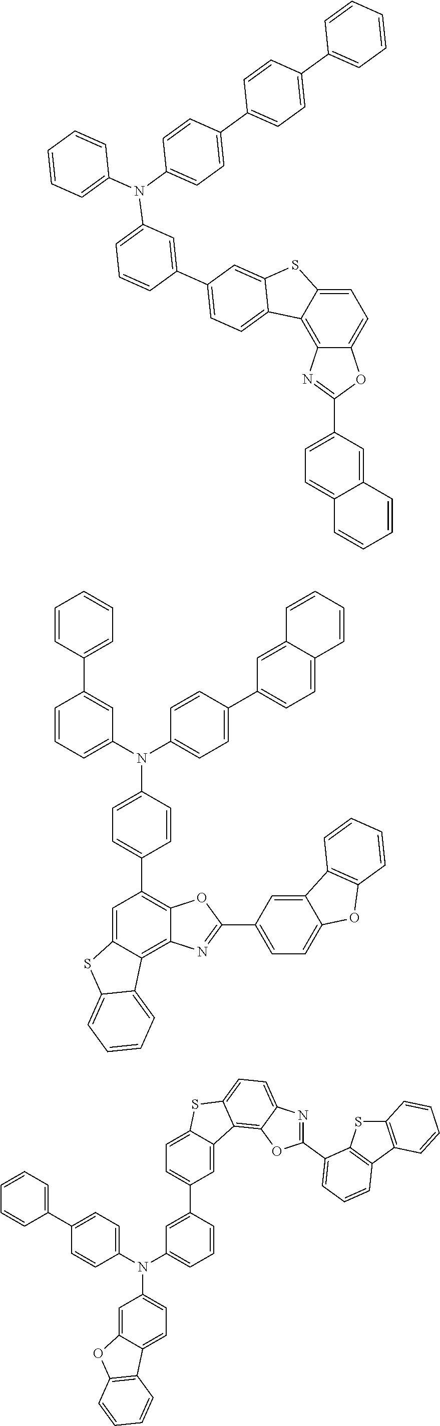 [SMILES: c1ccc(-c2ccc(-c3ccc(N(c4ccccc4)c4cccc(-c5ccc6c(c5)sc5ccc7oc(-c8ccc9ccccc9c8)nc7c56)c4)cc3)cc2)cc1 ; c1ccc(-c2cccc(N(c3ccc(-c4ccc5ccccc5c4)cc3)c3ccc(-c4cc5sc6ccccc6c5c5nc(-c6ccc7oc8ccccc8c7c6)oc45)cc3)c2)cc1 ; c1ccc(-c2ccc(N(c3cccc(-c4ccc5sc6ccc7nc(-c8cccc9c8sc8ccccc89)oc7c6c5c4)c3)c3ccc4c(c3)oc3ccccc34)cc2)cc1]

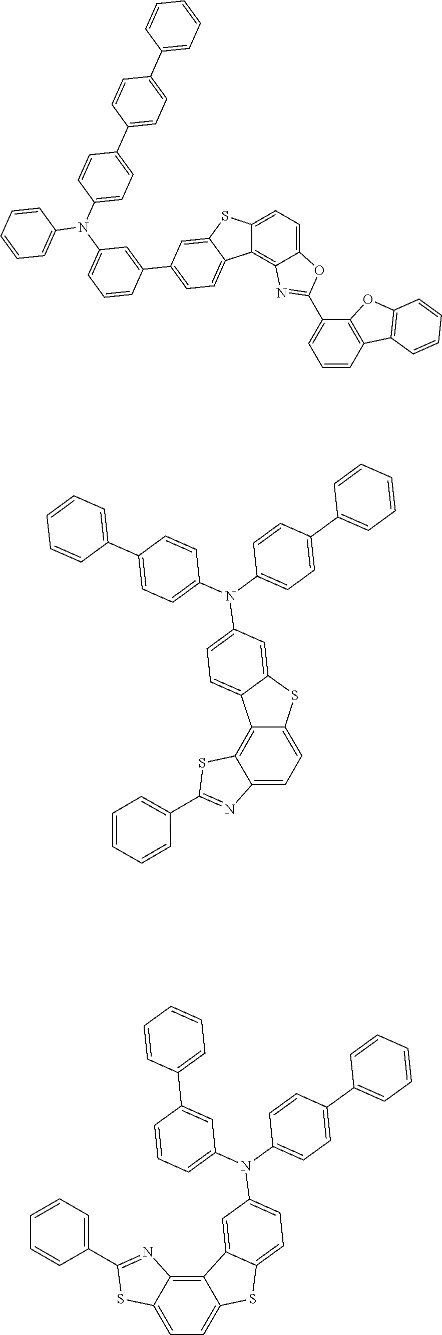 [SMILES: c1ccc(-c2ccc(-c3ccc(N(c4ccccc4)c4cccc(-c5ccc6c(c5)sc5ccc7oc(-c8cccc9c8oc8ccccc89)nc7c56)c4)cc3)cc2)cc1 ; c1ccc(-c2ccc(N(c3ccc(-c4ccccc4)cc3)c3ccc4c(c3)sc3ccc5nc(-c6ccccc6)sc5c34)cc2)cc1 ; c1ccc(-c2ccc(N(c3cccc(-c4ccccc4)c3)c3ccc4sc5ccc6sc(-c7ccccc7)nc6c5c4c3)cc2)cc1]

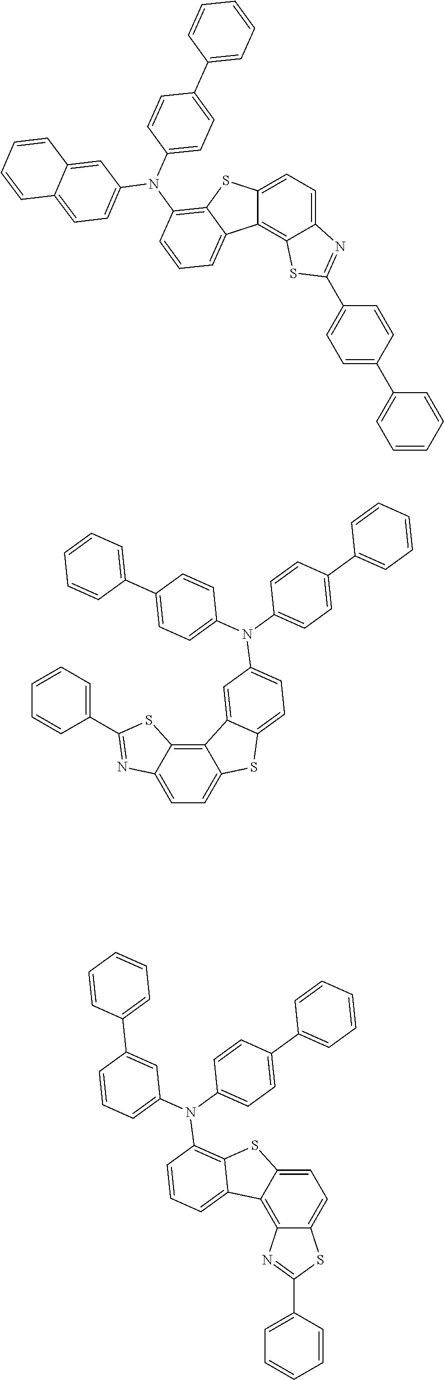 [SMILES: c1ccc(-c2ccc(-c3nc4ccc5sc6c(N(c7ccc(-c8ccccc8)cc7)c7ccc8ccccc8c7)cccc6c5c4s3)cc2)cc1 ; c1ccc(-c2ccc(N(c3ccc(-c4ccccc4)cc3)c3ccc4sc5ccc6nc(-c7ccccc7)sc6c5c4c3)cc2)cc1 ; c1ccc(-c2ccc(N(c3cccc(-c4ccccc4)c3)c3cccc4c3sc3ccc5sc(-c6ccccc6)nc5c34)cc2)cc1]

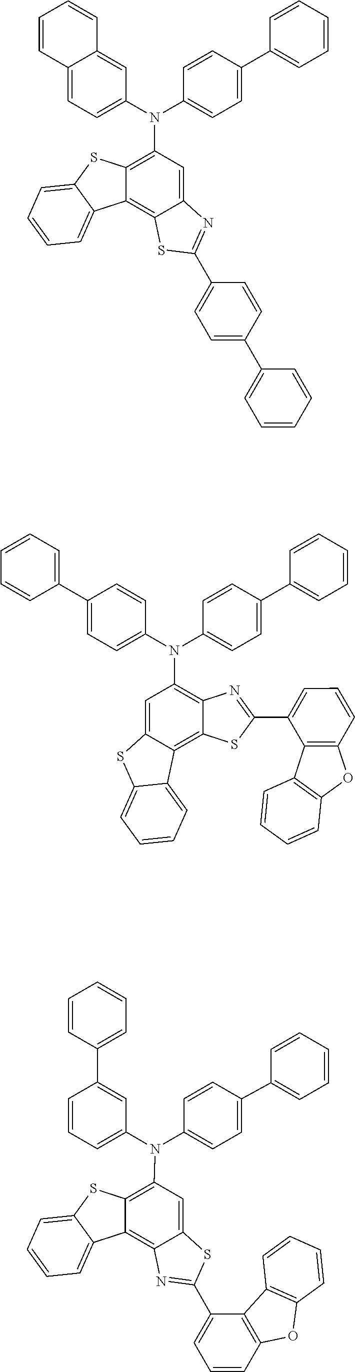 [SMILES: c1ccc(-c2ccc(-c3nc4cc(N(c5ccc(-c6ccccc6)cc5)c5ccc6ccccc6c5)c5sc6ccccc6c5c4s3)cc2)cc1 ; c1ccc(-c2ccc(N(c3ccc(-c4ccccc4)cc3)c3cc4sc5ccccc5c4c4sc(-c5cccc6oc7ccccc7c56)nc34)cc2)cc1 ; c1ccc(-c2ccc(N(c3cccc(-c4ccccc4)c3)c3cc4sc(-c5cccc6oc7ccccc7c56)nc4c4c3sc3ccccc34)cc2)cc1]

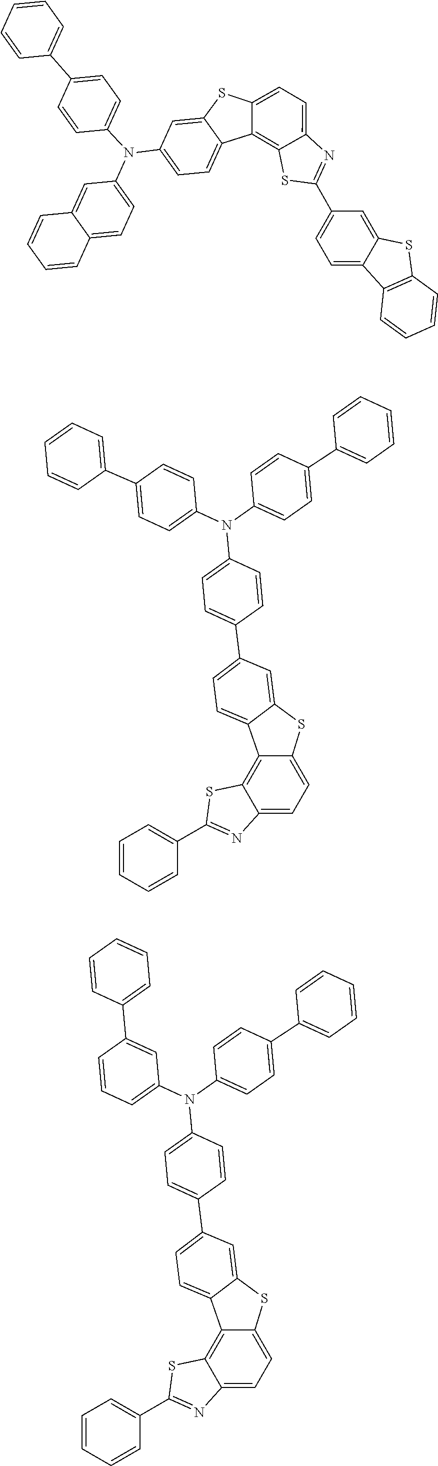 [SMILES: c1ccc(-c2ccc(N(c3ccc4ccccc4c3)c3ccc4c(c3)sc3ccc5nc(-c6ccc7c(c6)sc6ccccc67)sc5c34)cc2)cc1 ; c1ccc(-c2ccc(N(c3ccc(-c4ccccc4)cc3)c3ccc(-c4ccc5c(c4)sc4ccc6nc(-c7ccccc7)sc6c45)cc3)cc2)cc1 ; c1ccc(-c2ccc(N(c3ccc(-c4ccc5c(c4)sc4ccc6nc(-c7ccccc7)sc6c45)cc3)c3cccc(-c4ccccc4)c3)cc2)cc1]

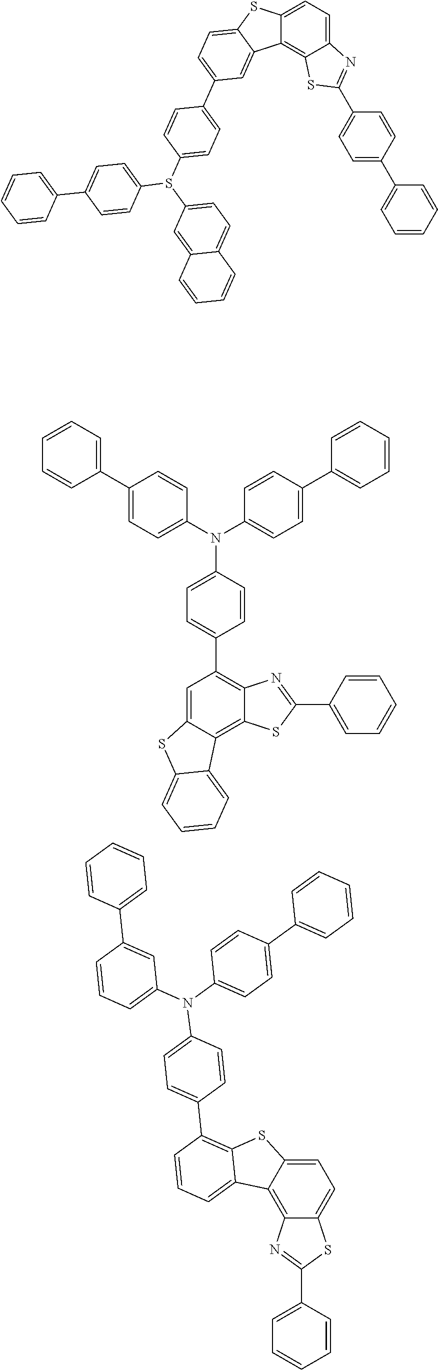

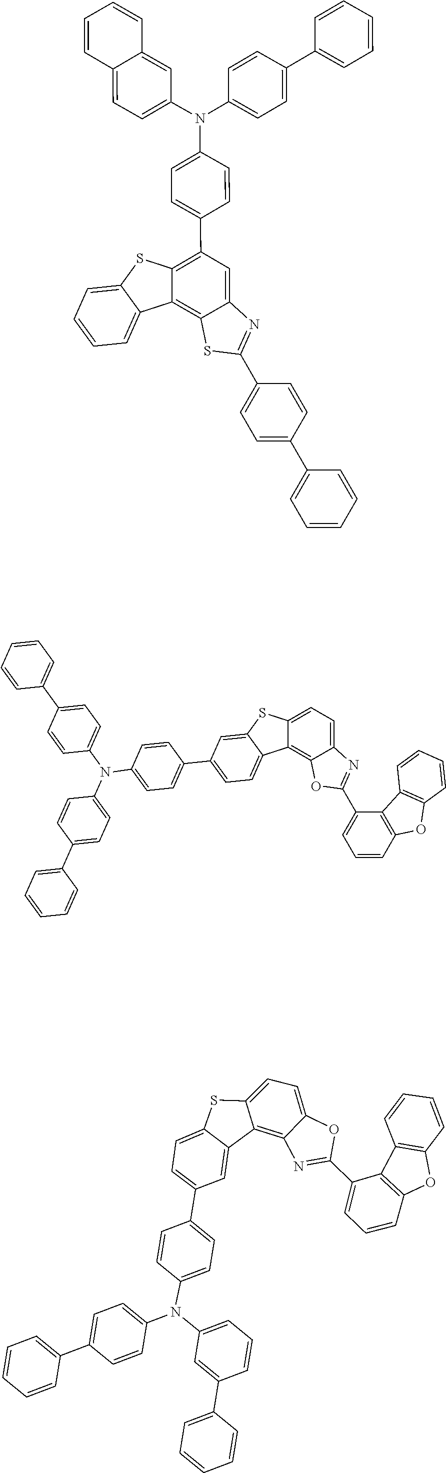 [SMILES: c1ccc(-c2ccc(-c3nc4cc(-c5ccc(N(c6ccc(-c7ccccc7)cc6)c6ccc7ccccc7c6)cc5)c5sc6ccccc6c5c4s3)cc2)cc1 ; c1ccc(-c2ccc(N(c3ccc(-c4ccccc4)cc3)c3ccc(-c4ccc5c(c4)sc4ccc6nc(-c7cccc8oc9ccccc9c78)oc6c45)cc3)cc2)cc1 ; c1ccc(-c2ccc(N(c3ccc(-c4ccc5sc6ccc7oc(-c8cccc9oc%10ccccc%10c89)nc7c6c5c4)cc3)c3cccc(-c4ccccc4)c3)cc2)cc1]

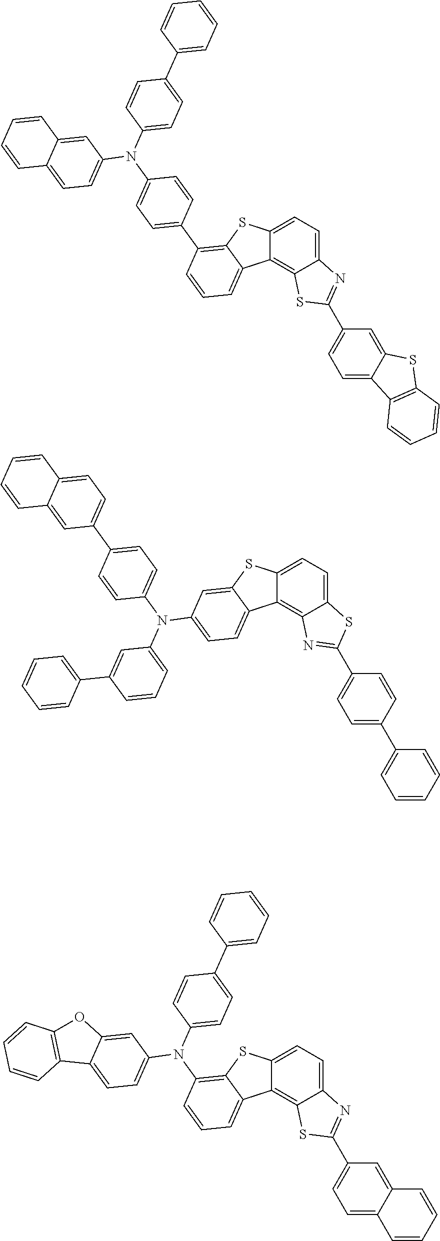 [SMILES: c1ccc(-c2ccc(N(c3ccc(-c4cccc5c4sc4ccc6nc(-c7ccc8c(c7)sc7ccccc78)sc6c45)cc3)c3ccc4ccccc4c3)cc2)cc1 ; c1ccc(-c2ccc(-c3nc4c(ccc5sc6cc(N(c7ccc(-c8ccc9ccccc9c8)cc7)c7cccc(-c8ccccc8)c7)ccc6c54)s3)cc2)cc1 ; c1ccc(-c2ccc(N(c3ccc4c(c3)oc3ccccc34)c3cccc4c3sc3ccc5nc(-c6ccc7ccccc7c6)sc5c34)cc2)cc1]

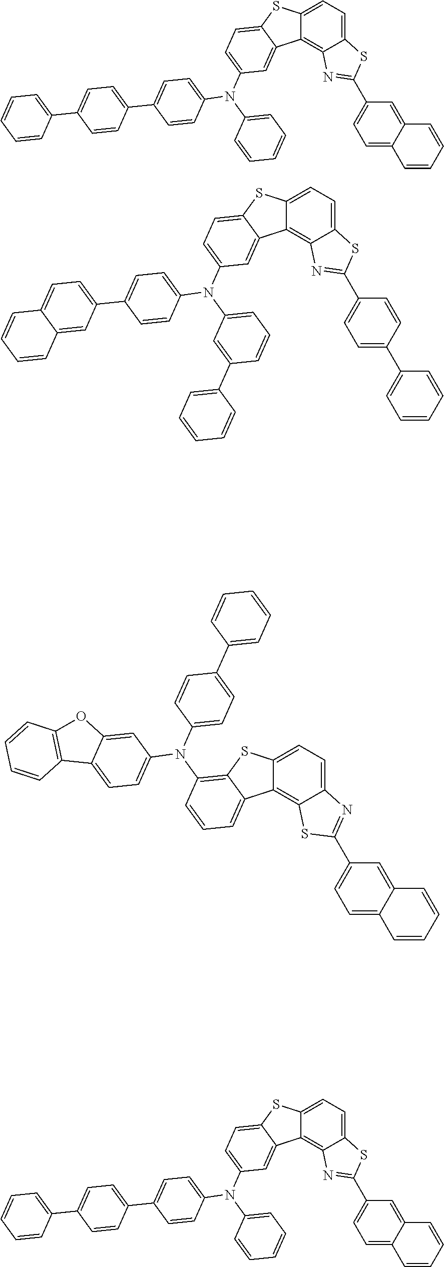

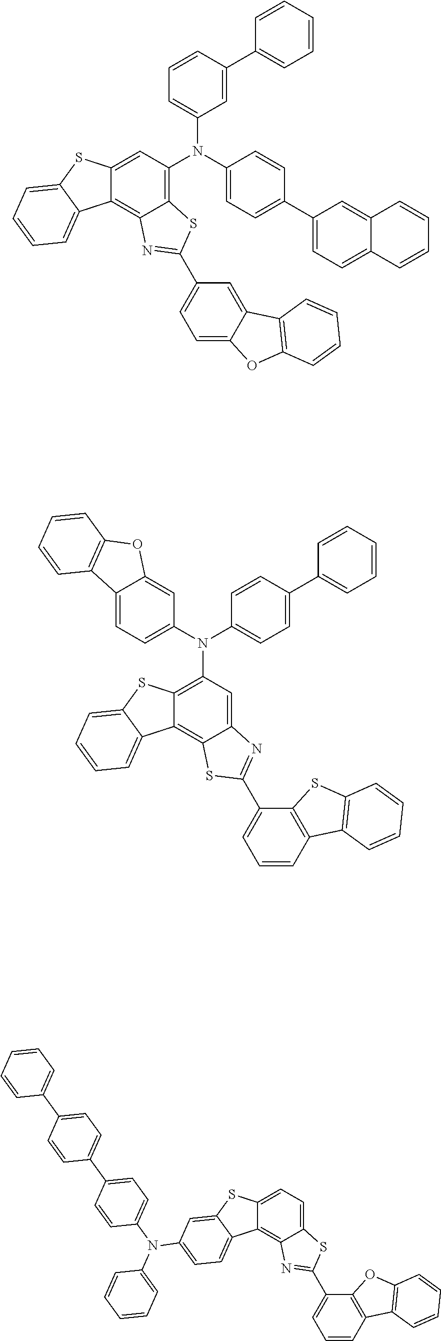 [SMILES: c1ccc(-c2cccc(N(c3ccc(-c4ccc5ccccc5c4)cc3)c3cc4sc5ccccc5c4c4nc(-c5ccc6oc7ccccc7c6c5)sc34)c2)cc1 ; c1ccc(-c2ccc(N(c3ccc4c(c3)oc3ccccc34)c3cc4nc(-c5cccc6c5sc5ccccc56)sc4c4c3sc3ccccc34)cc2)cc1 ; c1ccc(-c2ccc(-c3ccc(N(c4ccccc4)c4ccc5c(c4)sc4ccc6sc(-c7cccc8c7oc7ccccc78)nc6c45)cc3)cc2)cc1]

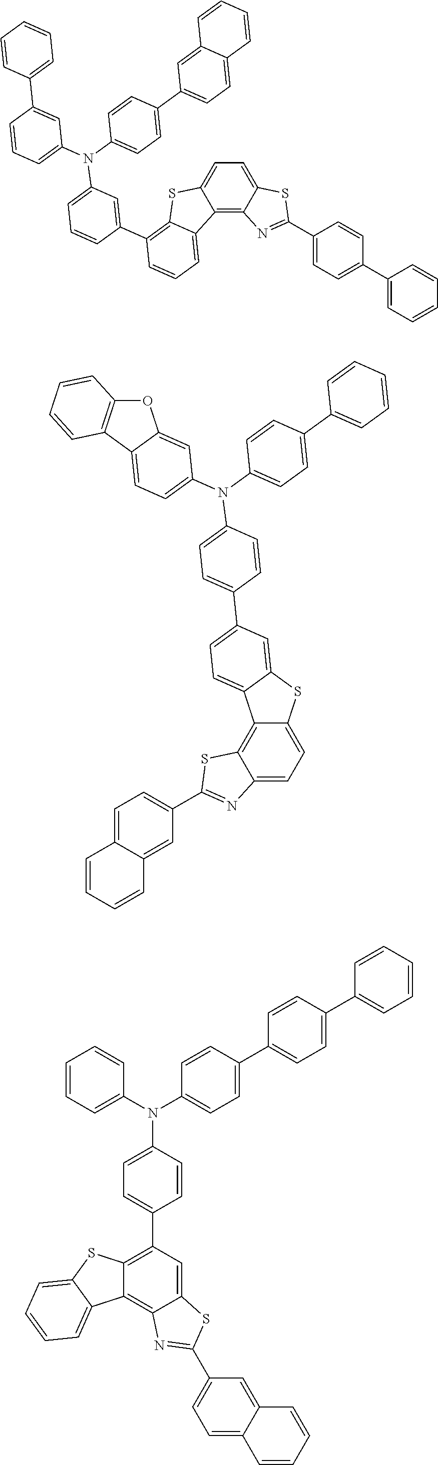 [SMILES: c1ccc(-c2ccc(-c3nc4c(ccc5sc6c(-c7cccc(N(c8ccc(-c9ccc%10ccccc%10c9)cc8)c8cccc(-c9ccccc9)c8)c7)cccc6c54)s3)cc2)cc1 ; c1ccc(-c2ccc(N(c3ccc(-c4ccc5c(c4)sc4ccc6nc(-c7ccc8ccccc8c7)sc6c45)cc3)c3ccc4c(c3)oc3ccccc34)cc2)cc1 ; c1ccc(-c2ccc(-c3ccc(N(c4ccccc4)c4ccc(-c5cc6sc(-c7ccc8ccccc8c7)nc6c6c5sc5ccccc56)cc4)cc3)cc2)cc1]

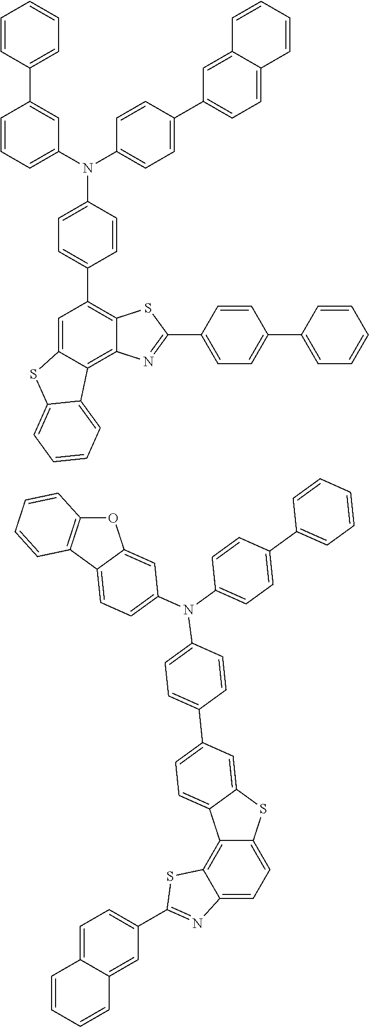

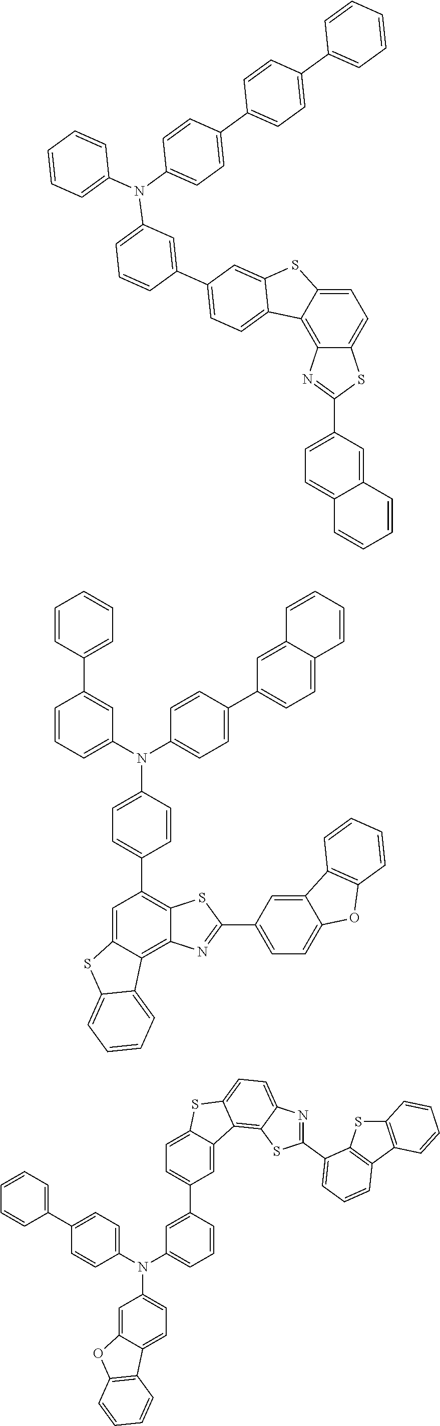 [SMILES: c1ccc(-c2ccc(-c3ccc(N(c4ccccc4)c4cccc(-c5ccc6c(c5)sc5ccc7sc(-c8ccc9ccccc9c8)nc7c56)c4)cc3)cc2)cc1 ; c1ccc(-c2cccc(N(c3ccc(-c4ccc5ccccc5c4)cc3)c3ccc(-c4cc5sc6ccccc6c5c5nc(-c6ccc7oc8ccccc8c7c6)sc45)cc3)c2)cc1 ; c1ccc(-c2ccc(N(c3cccc(-c4ccc5sc6ccc7nc(-c8cccc9c8sc8ccccc89)sc7c6c5c4)c3)c3ccc4c(c3)oc3ccccc34)cc2)cc1]

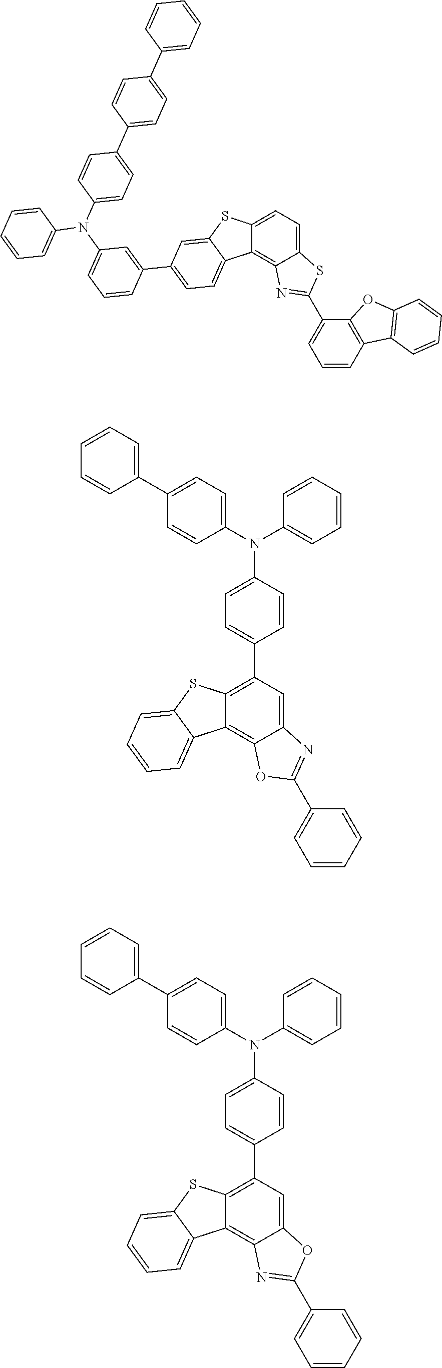 [SMILES: c1ccc(-c2ccc(-c3ccc(N(c4ccccc4)c4cccc(-c5ccc6c(c5)sc5ccc7sc(-c8cccc9c8oc8ccccc89)nc7c56)c4)cc3)cc2)cc1 ; c1ccc(-c2ccc(N(c3ccccc3)c3ccc(-c4cc5nc(-c6ccccc6)oc5c5c4sc4ccccc45)cc3)cc2)cc1 ; c1ccc(-c2ccc(N(c3ccccc3)c3ccc(-c4cc5oc(-c6ccccc6)nc5c5c4sc4ccccc45)cc3)cc2)cc1]

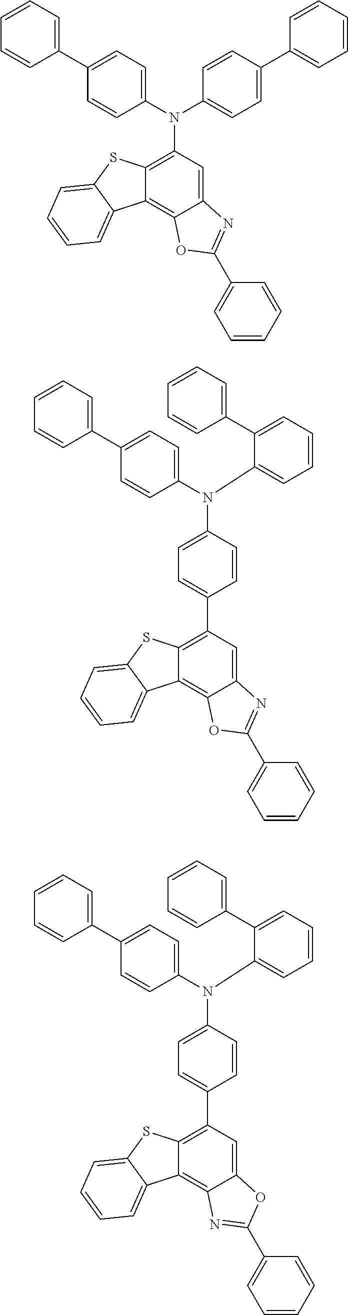

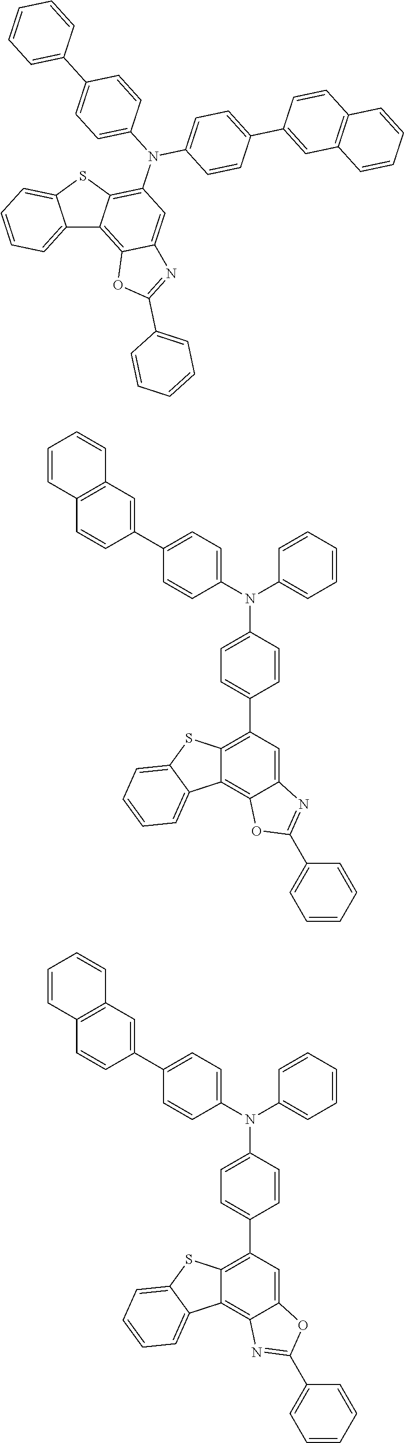 [SMILES: c1ccc(-c2ccc(N(c3ccc(-c4ccc5ccccc5c4)cc3)c3cc4nc(-c5ccccc5)oc4c4c3sc3ccccc34)cc2)cc1 ; c1ccc(-c2nc3cc(-c4ccc(N(c5ccccc5)c5ccc(-c6ccc7ccccc7c6)cc5)cc4)c4sc5ccccc5c4c3o2)cc1 ; c1ccc(-c2nc3c(cc(-c4ccc(N(c5ccccc5)c5ccc(-c6ccc7ccccc7c6)cc5)cc4)c4sc5ccccc5c43)o2)cc1]

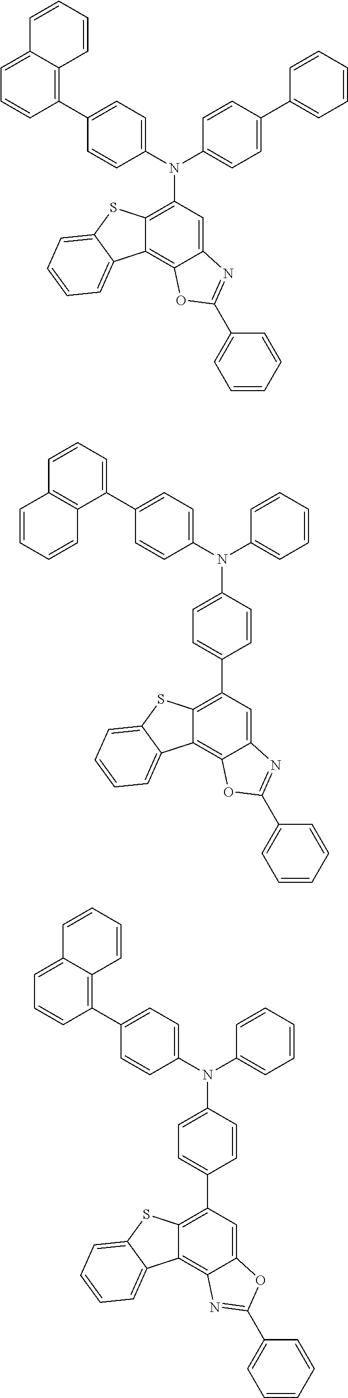 [SMILES: c1ccc(-c2ccc(N(c3ccc(-c4cccc5ccccc45)cc3)c3cc4nc(-c5ccccc5)oc4c4c3sc3ccccc34)cc2)cc1 ; c1ccc(-c2nc3cc(-c4ccc(N(c5ccccc5)c5ccc(-c6cccc7ccccc67)cc5)cc4)c4sc5ccccc5c4c3o2)cc1 ; c1ccc(-c2nc3c(cc(-c4ccc(N(c5ccccc5)c5ccc(-c6cccc7ccccc67)cc5)cc4)c4sc5ccccc5c43)o2)cc1]

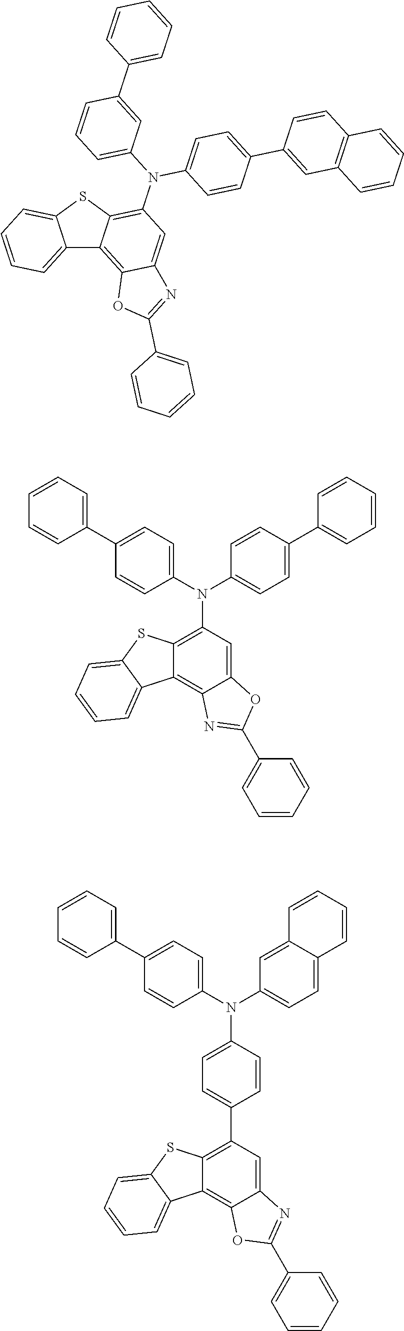 [SMILES: c1ccc(-c2cccc(N(c3ccc(-c4ccc5ccccc5c4)cc3)c3cc4nc(-c5ccccc5)oc4c4c3sc3ccccc34)c2)cc1 ; c1ccc(-c2ccc(N(c3ccc(-c4ccccc4)cc3)c3cc4oc(-c5ccccc5)nc4c4c3sc3ccccc34)cc2)cc1 ; c1ccc(-c2ccc(N(c3ccc(-c4cc5nc(-c6ccccc6)oc5c5c4sc4ccccc45)cc3)c3ccc4ccccc4c3)cc2)cc1]

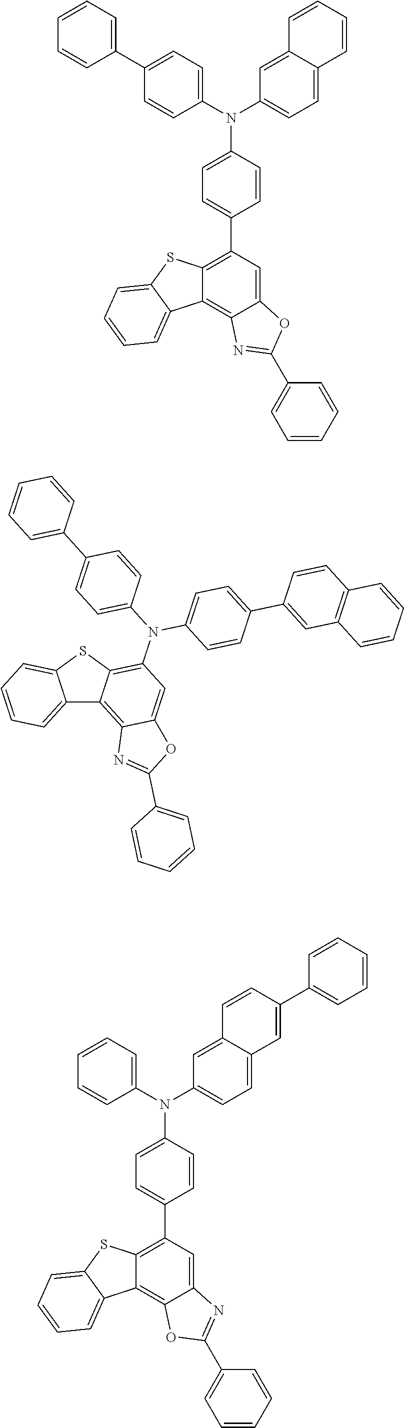 [SMILES: c1ccc(-c2ccc(N(c3ccc(-c4cc5oc(-c6ccccc6)nc5c5c4sc4ccccc45)cc3)c3ccc4ccccc4c3)cc2)cc1 ; c1ccc(-c2ccc(N(c3ccc(-c4ccc5ccccc5c4)cc3)c3cc4oc(-c5ccccc5)nc4c4c3sc3ccccc34)cc2)cc1 ; c1ccc(-c2ccc3cc(N(c4ccccc4)c4ccc(-c5cc6nc(-c7ccccc7)oc6c6c5sc5ccccc56)cc4)ccc3c2)cc1]

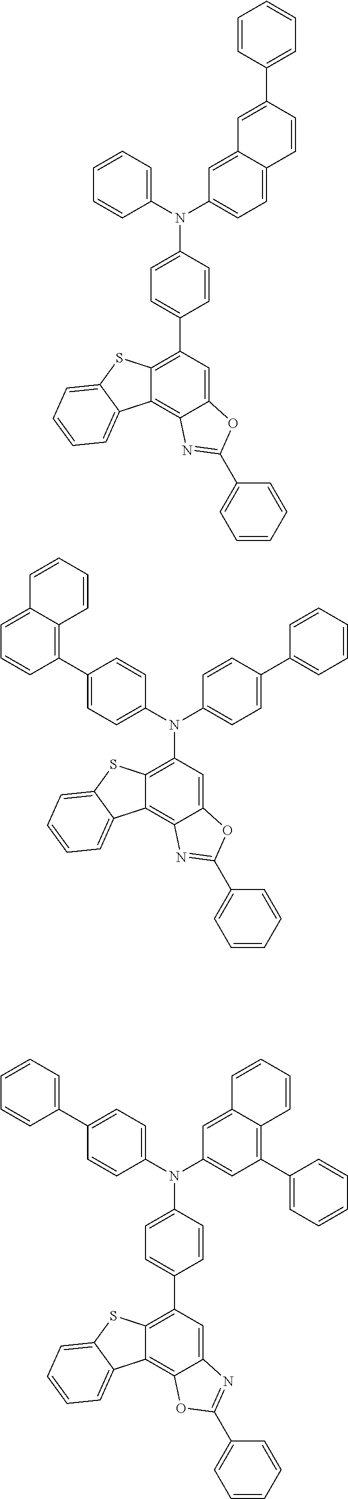 [SMILES: c1ccc(-c2ccc3ccc(N(c4ccccc4)c4ccc(-c5cc6oc(-c7ccccc7)nc6c6c5sc5ccccc56)cc4)cc3c2)cc1 ; c1ccc(-c2ccc(N(c3ccc(-c4cccc5ccccc45)cc3)c3cc4oc(-c5ccccc5)nc4c4c3sc3ccccc34)cc2)cc1 ; c1ccc(-c2ccc(N(c3ccc(-c4cc5nc(-c6ccccc6)oc5c5c4sc4ccccc45)cc3)c3cc(-c4ccccc4)c4ccccc4c3)cc2)cc1]

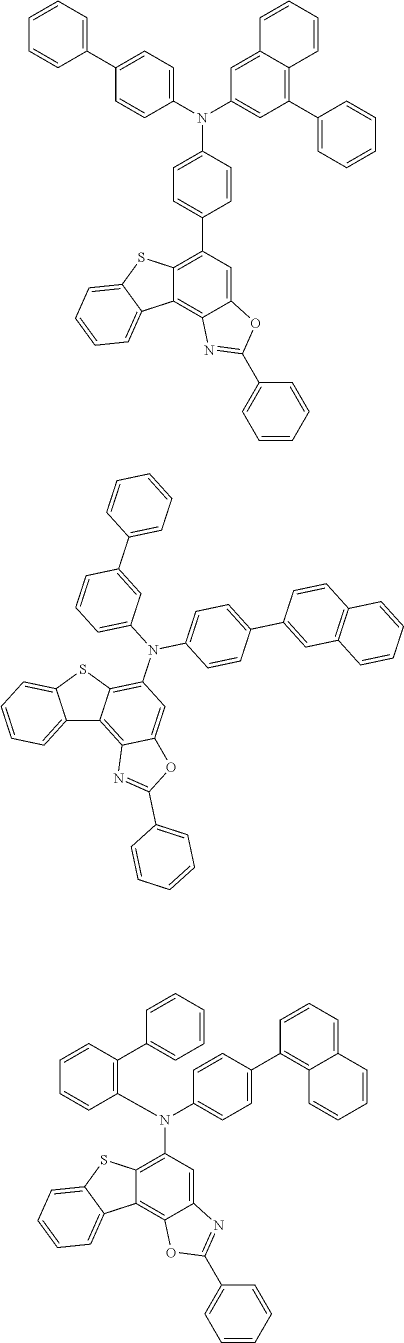 [SMILES: c1ccc(-c2ccc(N(c3ccc(-c4cc5oc(-c6ccccc6)nc5c5c4sc4ccccc45)cc3)c3cc(-c4ccccc4)c4ccccc4c3)cc2)cc1 ; c1ccc(-c2cccc(N(c3ccc(-c4ccc5ccccc5c4)cc3)c3cc4oc(-c5ccccc5)nc4c4c3sc3ccccc34)c2)cc1 ; c1ccc(-c2nc3cc(N(c4ccc(-c5cccc6ccccc56)cc4)c4ccccc4-c4ccccc4)c4sc5ccccc5c4c3o2)cc1]

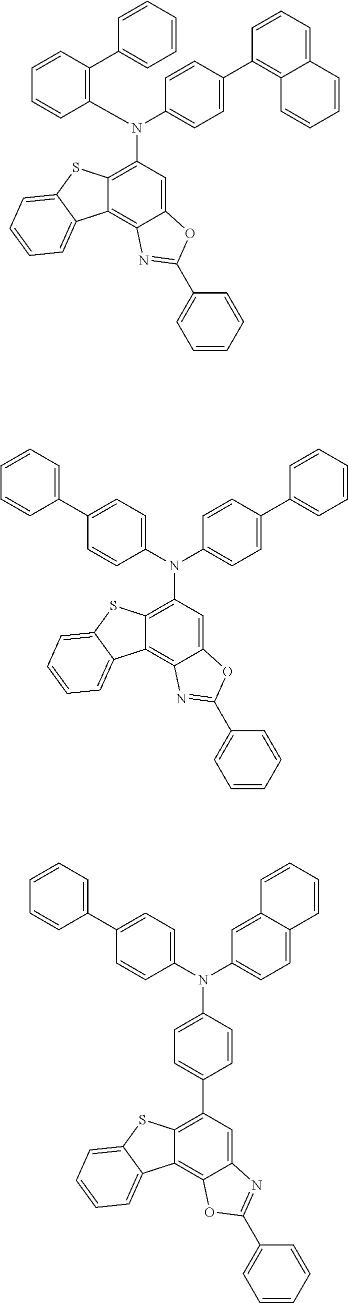 [SMILES: c1ccc(-c2nc3c(cc(N(c4ccc(-c5cccc6ccccc56)cc4)c4ccccc4-c4ccccc4)c4sc5ccccc5c43)o2)cc1 ; c1ccc(-c2ccc(N(c3ccc(-c4ccccc4)cc3)c3cc4oc(-c5ccccc5)nc4c4c3sc3ccccc34)cc2)cc1 ; c1ccc(-c2ccc(N(c3ccc(-c4cc5nc(-c6ccccc6)oc5c5c4sc4ccccc45)cc3)c3ccc4ccccc4c3)cc2)cc1]

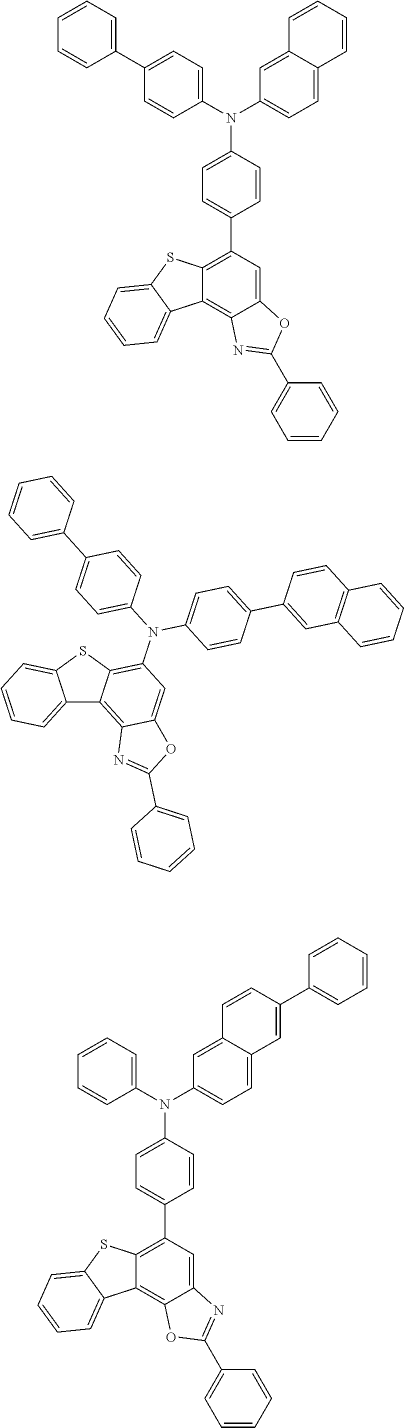 [SMILES: c1ccc(-c2ccc(N(c3ccc(-c4cc5oc(-c6ccccc6)nc5c5c4sc4ccccc45)cc3)c3ccc4ccccc4c3)cc2)cc1 ; c1ccc(-c2ccc(N(c3ccc(-c4ccc5ccccc5c4)cc3)c3cc4oc(-c5ccccc5)nc4c4c3sc3ccccc34)cc2)cc1 ; c1ccc(-c2ccc3cc(N(c4ccccc4)c4ccc(-c5cc6nc(-c7ccccc7)oc6c6c5sc5ccccc56)cc4)ccc3c2)cc1]

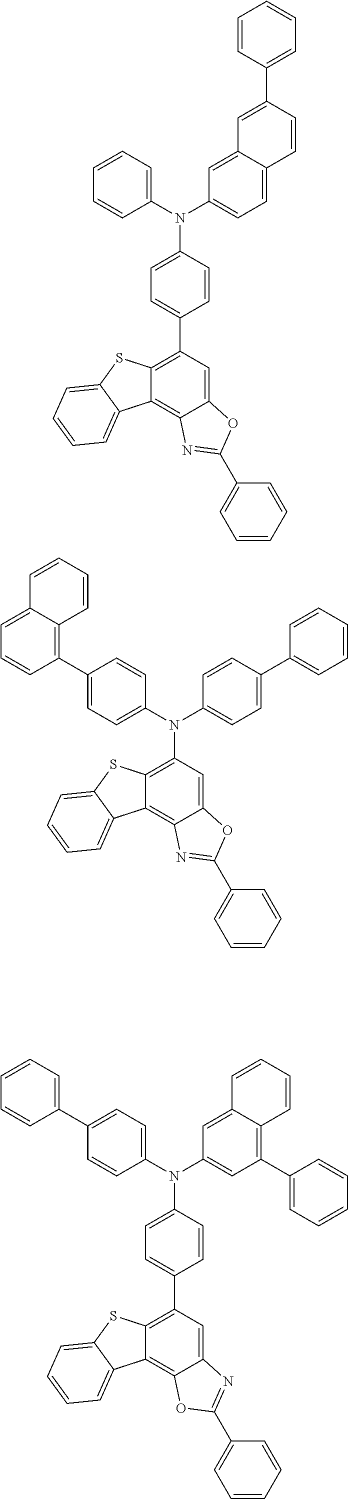 [SMILES: c1ccc(-c2ccc3ccc(N(c4ccccc4)c4ccc(-c5cc6oc(-c7ccccc7)nc6c6c5sc5ccccc56)cc4)cc3c2)cc1 ; c1ccc(-c2ccc(N(c3ccc(-c4cccc5ccccc45)cc3)c3cc4oc(-c5ccccc5)nc4c4c3sc3ccccc34)cc2)cc1 ; c1ccc(-c2ccc(N(c3ccc(-c4cc5nc(-c6ccccc6)oc5c5c4sc4ccccc45)cc3)c3cc(-c4ccccc4)c4ccccc4c3)cc2)cc1]

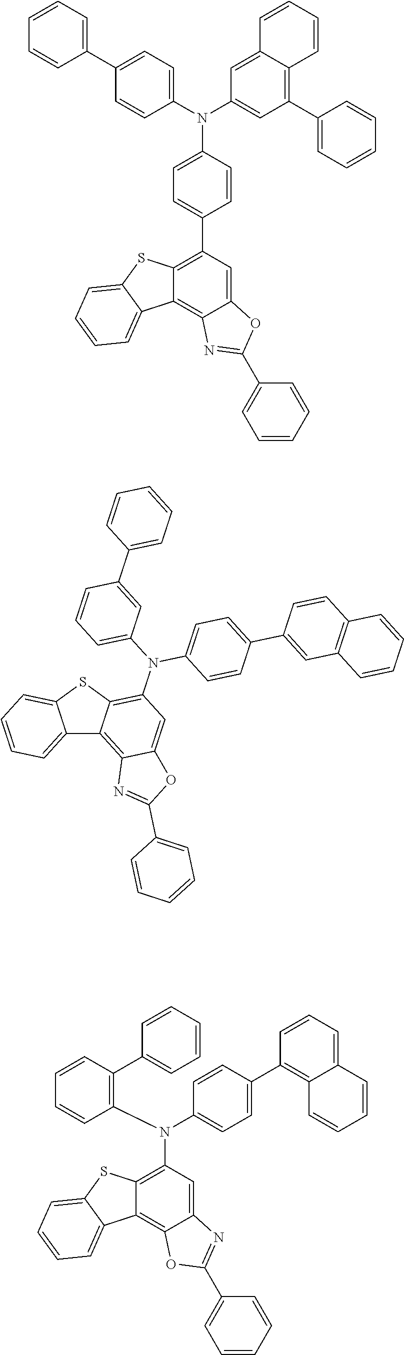 [SMILES: c1ccc(-c2ccc(N(c3ccc(-c4cc5oc(-c6ccccc6)nc5c5c4sc4ccccc45)cc3)c3cc(-c4ccccc4)c4ccccc4c3)cc2)cc1 ; c1ccc(-c2cccc(N(c3ccc(-c4ccc5ccccc5c4)cc3)c3cc4oc(-c5ccccc5)nc4c4c3sc3ccccc34)c2)cc1 ; c1ccc(-c2nc3cc(N(c4ccc(-c5cccc6ccccc56)cc4)c4ccccc4-c4ccccc4)c4sc5ccccc5c4c3o2)cc1]

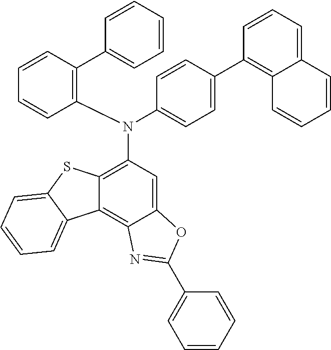 [SMILES: c1ccc(-c2nc3c(cc(N(c4ccc(-c5cccc6ccccc56)cc4)c4ccccc4-c4ccccc4)c4sc5ccccc5c43)o2)cc1]

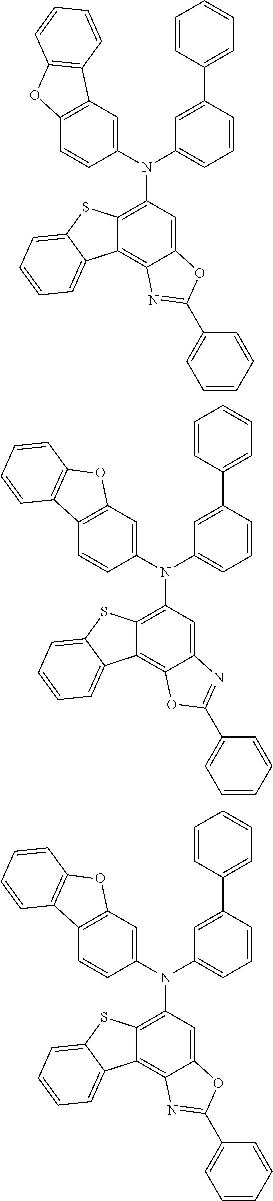

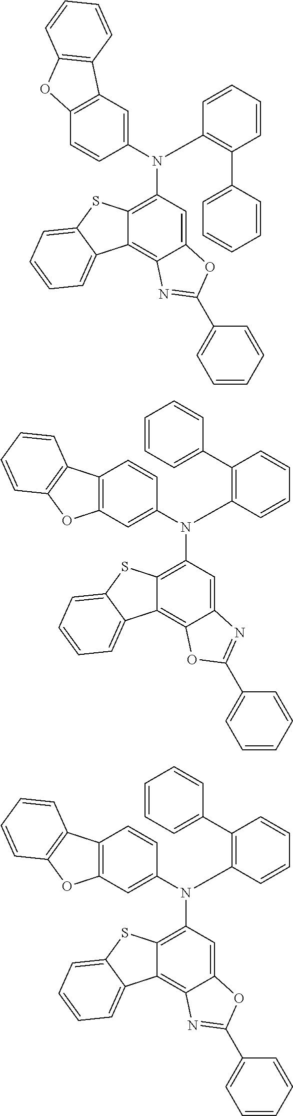

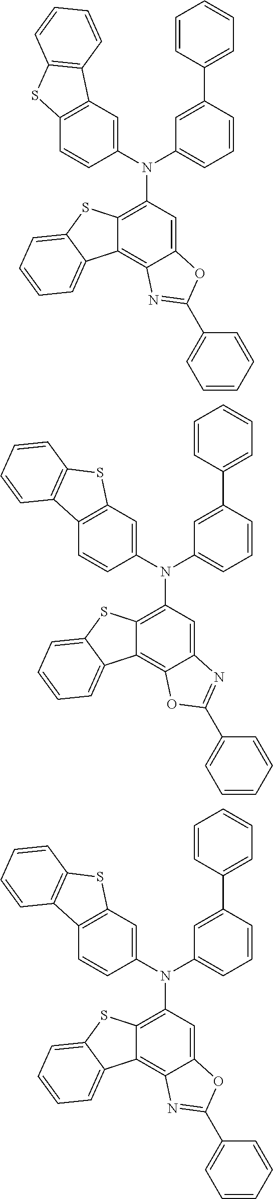

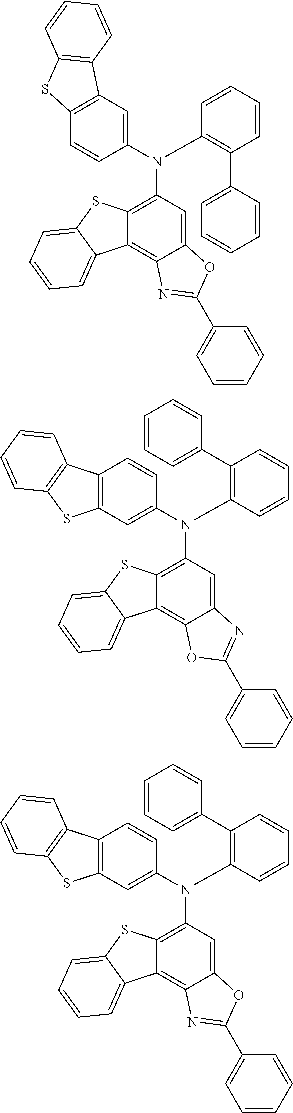

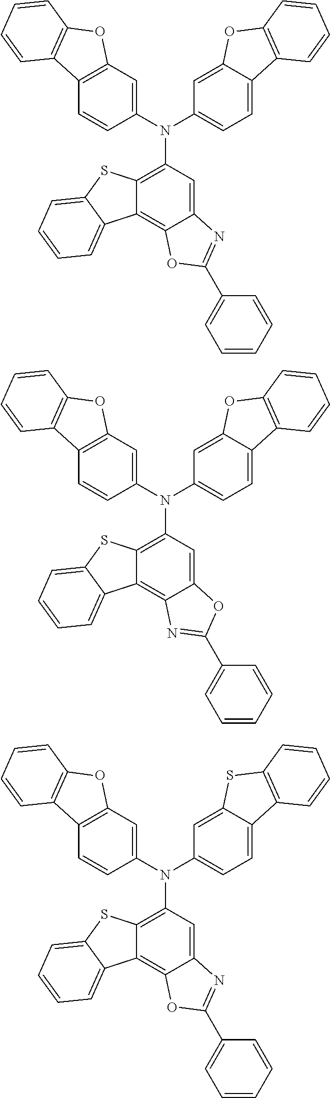

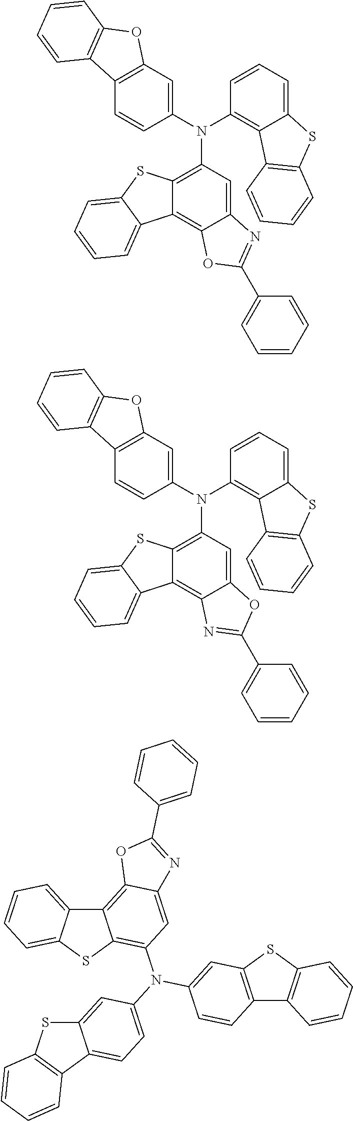 [SMILES: c1ccc(-c2nc3cc(N(c4ccc5c(c4)oc4ccccc45)c4cccc5sc6ccccc6c45)c4sc5ccccc5c4c3o2)cc1 ; c1ccc(-c2nc3c(cc(N(c4ccc5c(c4)oc4ccccc45)c4cccc5sc6ccccc6c45)c4sc5ccccc5c43)o2)cc1 ; c1ccc(-c2nc3cc(N(c4ccc5c(c4)sc4ccccc45)c4ccc5c(c4)sc4ccccc45)c4sc5ccccc5c4c3o2)cc1]

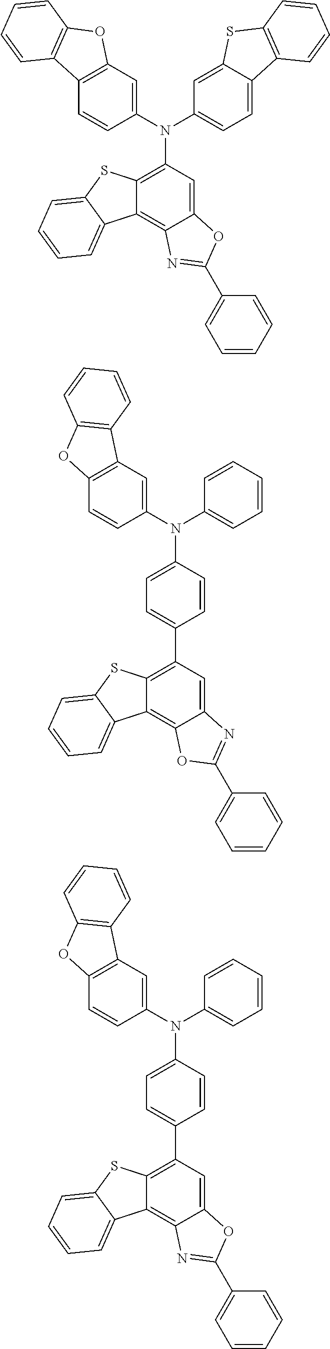 [SMILES: c1ccc(-c2nc3c(cc(N(c4ccc5c(c4)oc4ccccc45)c4ccc5c(c4)sc4ccccc45)c4sc5ccccc5c43)o2)cc1 ; c1ccc(-c2nc3cc(-c4ccc(N(c5ccccc5)c5ccc6oc7ccccc7c6c5)cc4)c4sc5ccccc5c4c3o2)cc1 ; c1ccc(-c2nc3c(cc(-c4ccc(N(c5ccccc5)c5ccc6oc7ccccc7c6c5)cc4)c4sc5ccccc5c43)o2)cc1]

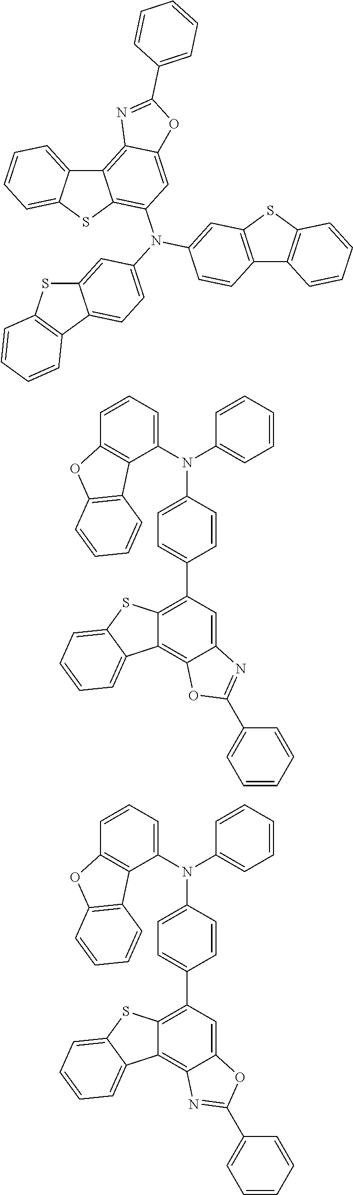

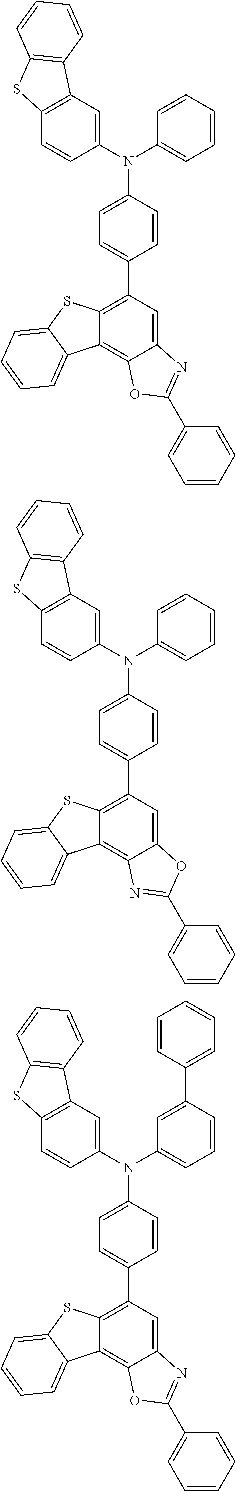

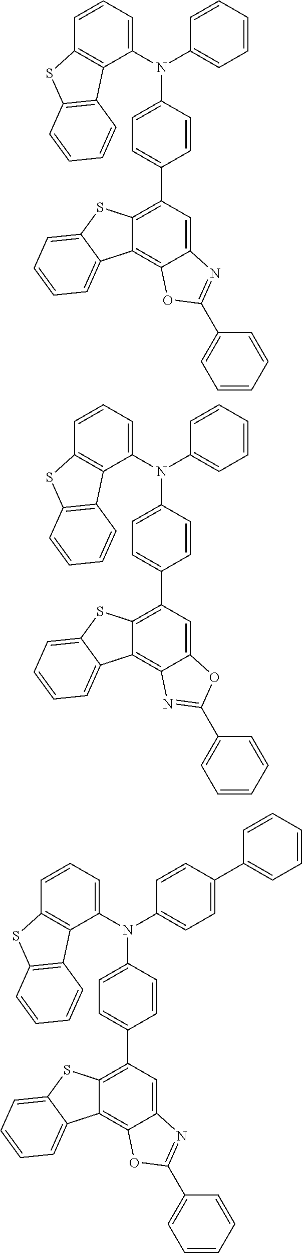

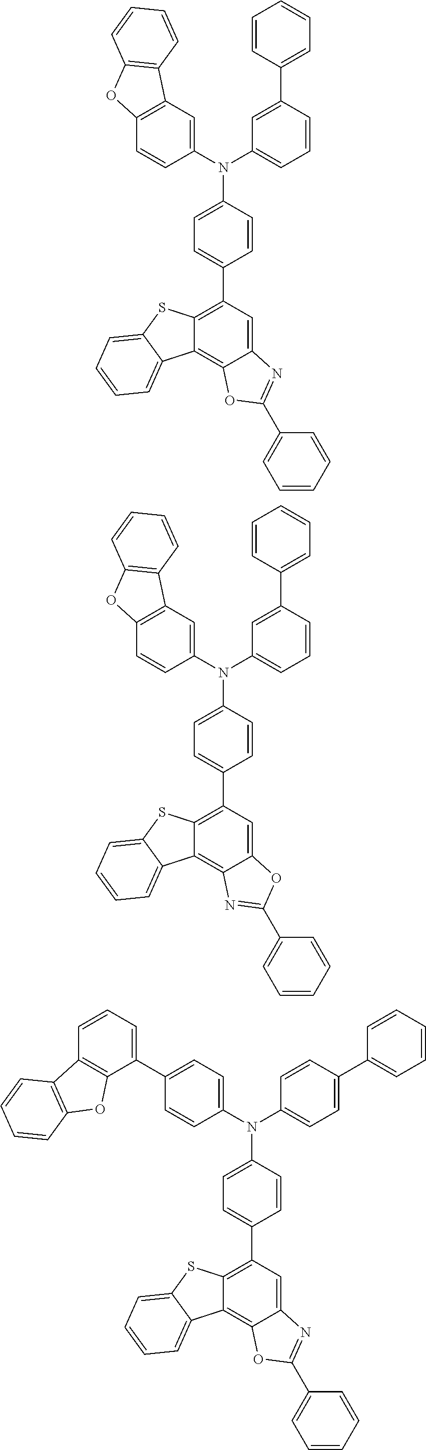

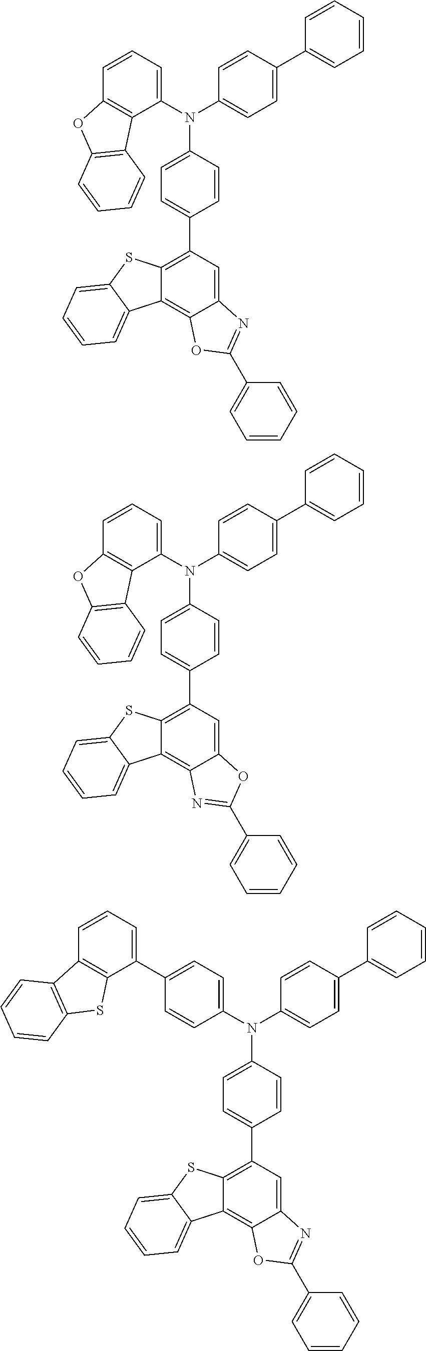

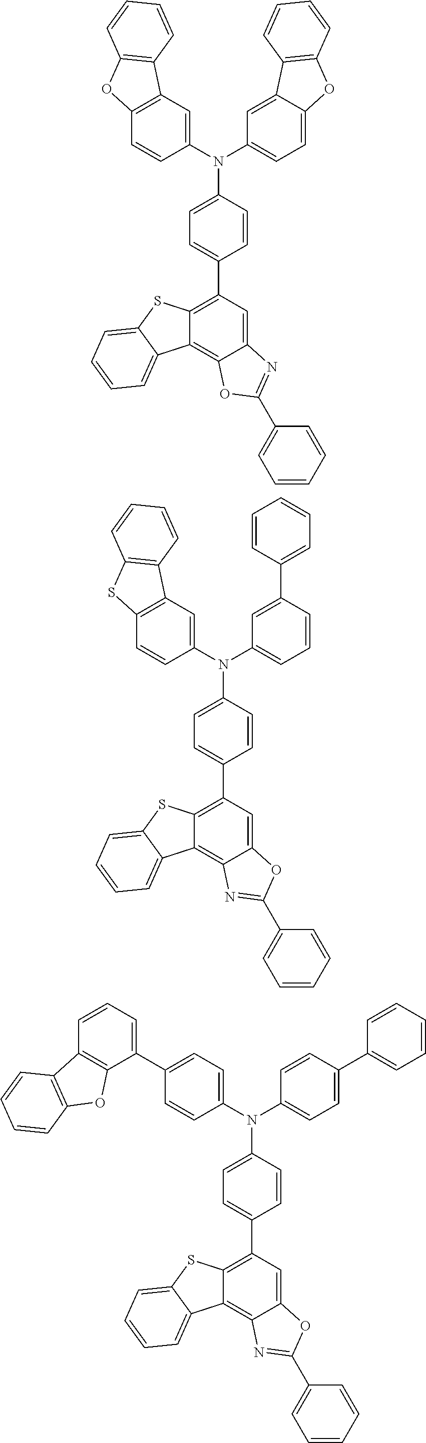

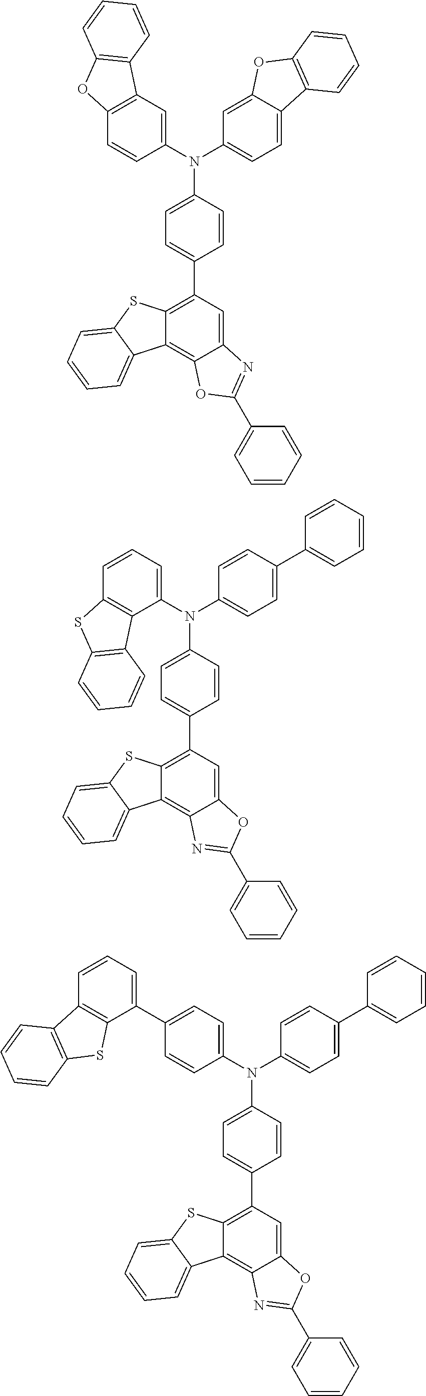

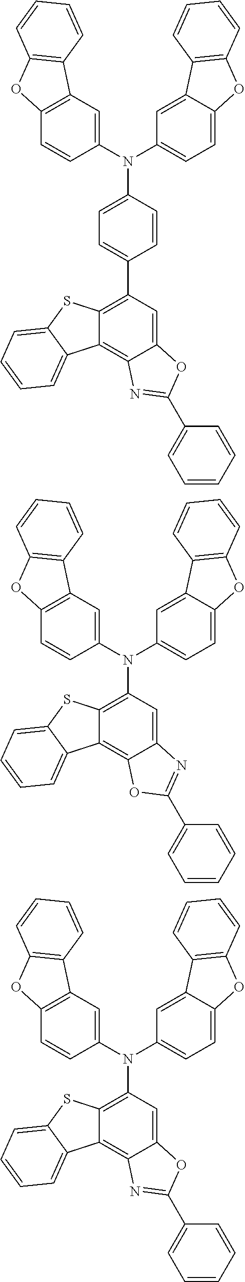

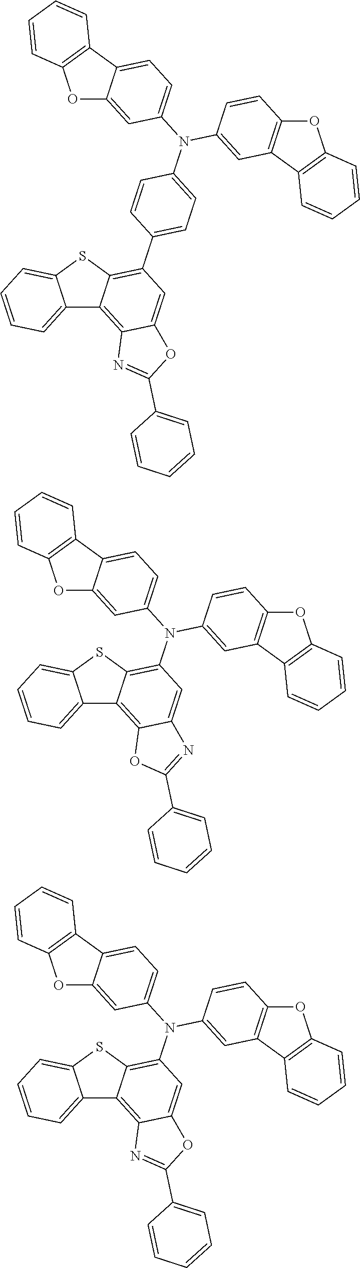 [SMILES: c1ccc(-c2nc3c(cc(-c4ccc(N(c5ccc6c(c5)oc5ccccc56)c5ccc6oc7ccccc7c6c5)cc4)c4sc5ccccc5c43)o2)cc1 ; c1ccc(-c2nc3cc(N(c4ccc5c(c4)oc4ccccc45)c4ccc5oc6ccccc6c5c4)c4sc5ccccc5c4c3o2)cc1 ; c1ccc(-c2nc3c(cc(N(c4ccc5c(c4)oc4ccccc45)c4ccc5oc6ccccc6c5c4)c4sc5ccccc5c43)o2)cc1]

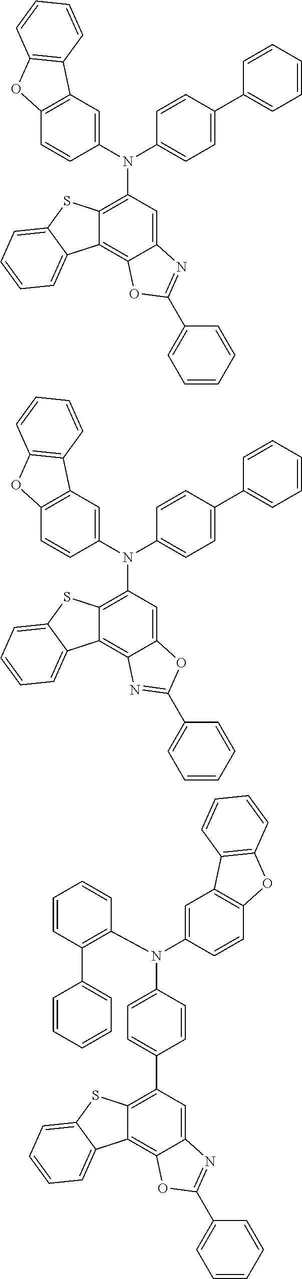

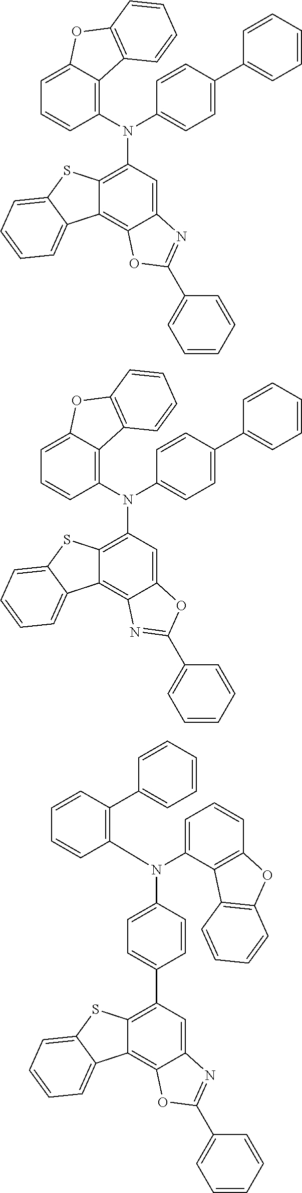

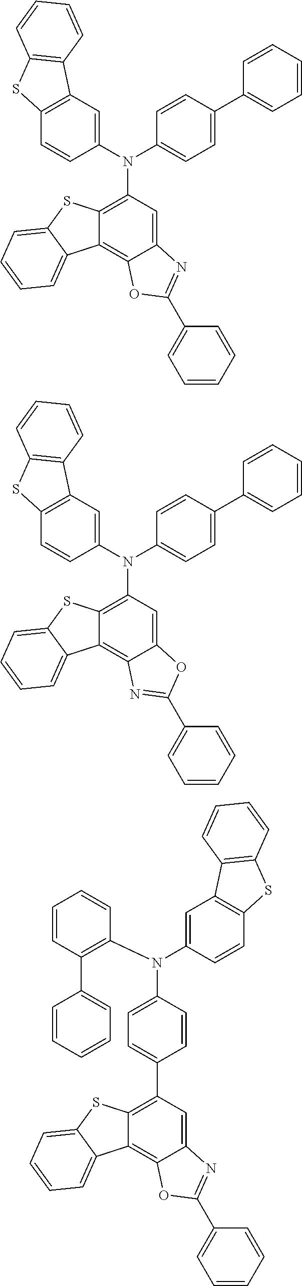

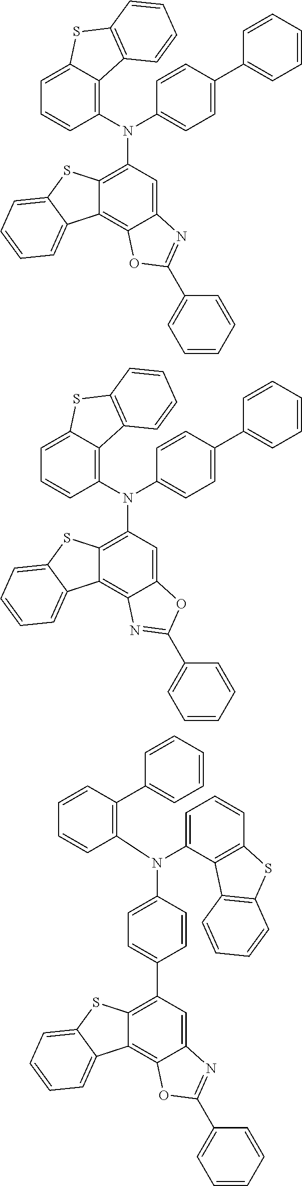

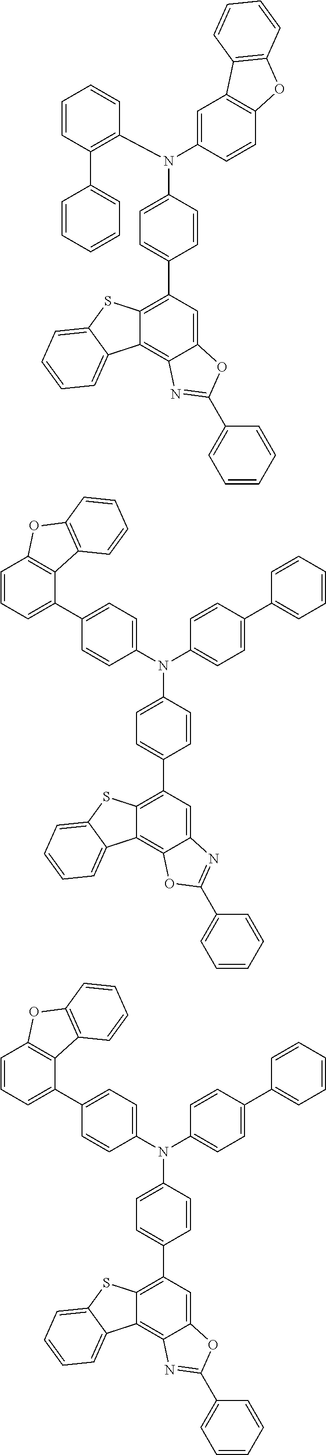 [SMILES: c1ccc(-c2nc3c(cc(-c4ccc(N(c5ccc6oc7ccccc7c6c5)c5ccccc5-c5ccccc5)cc4)c4sc5ccccc5c43)o2)cc1 ; c1ccc(-c2ccc(N(c3ccc(-c4cc5nc(-c6ccccc6)oc5c5c4sc4ccccc45)cc3)c3ccc(-c4cccc5oc6ccccc6c45)cc3)cc2)cc1 ; c1ccc(-c2ccc(N(c3ccc(-c4cc5oc(-c6ccccc6)nc5c5c4sc4ccccc45)cc3)c3ccc(-c4cccc5oc6ccccc6c45)cc3)cc2)cc1]

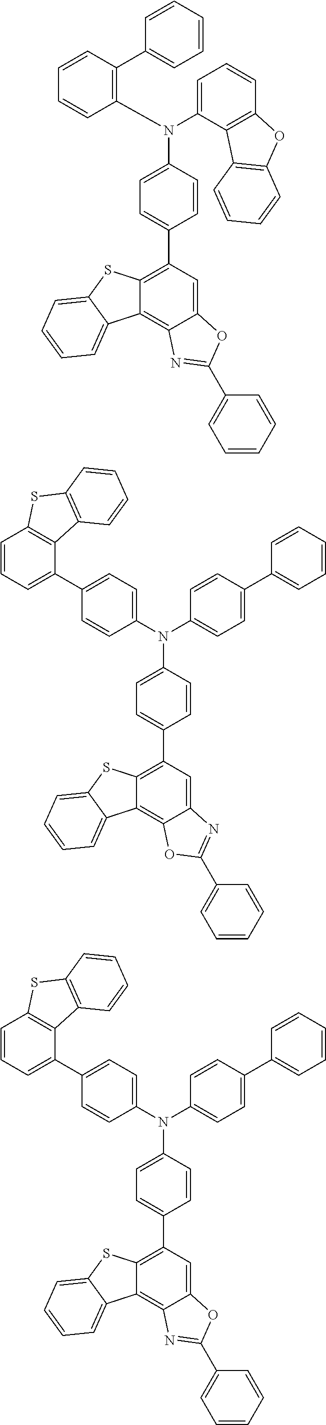 [SMILES: c1ccc(-c2nc3c(cc(-c4ccc(N(c5ccccc5-c5ccccc5)c5cccc6oc7ccccc7c56)cc4)c4sc5ccccc5c43)o2)cc1 ; c1ccc(-c2ccc(N(c3ccc(-c4cc5nc(-c6ccccc6)oc5c5c4sc4ccccc45)cc3)c3ccc(-c4cccc5sc6ccccc6c45)cc3)cc2)cc1 ; c1ccc(-c2ccc(N(c3ccc(-c4cc5oc(-c6ccccc6)nc5c5c4sc4ccccc45)cc3)c3ccc(-c4cccc5sc6ccccc6c45)cc3)cc2)cc1]

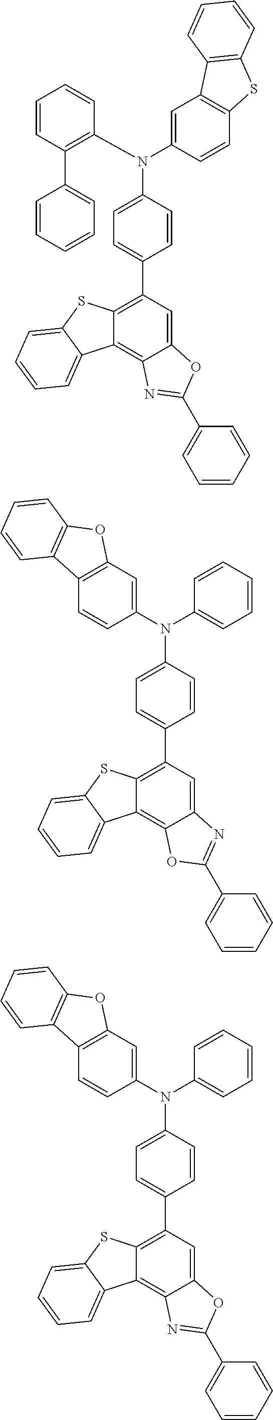 [SMILES: c1ccc(-c2nc3c(cc(-c4ccc(N(c5ccc6sc7ccccc7c6c5)c5ccccc5-c5ccccc5)cc4)c4sc5ccccc5c43)o2)cc1 ; c1ccc(-c2nc3cc(-c4ccc(N(c5ccccc5)c5ccc6c(c5)oc5ccccc56)cc4)c4sc5ccccc5c4c3o2)cc1 ; c1ccc(-c2nc3c(cc(-c4ccc(N(c5ccccc5)c5ccc6c(c5)oc5ccccc56)cc4)c4sc5ccccc5c43)o2)cc1]

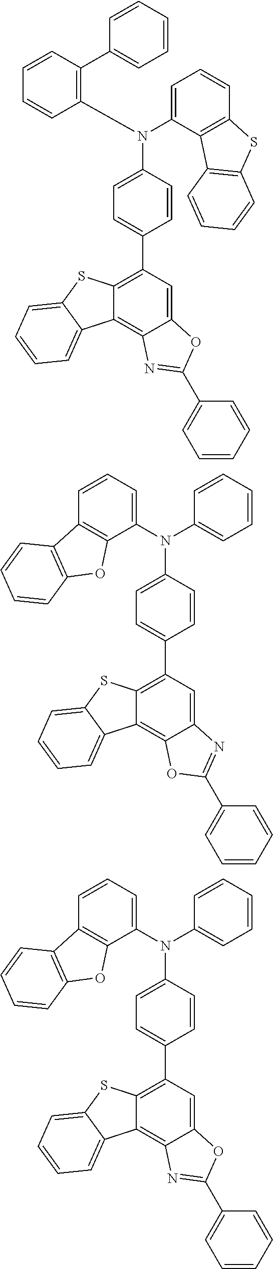

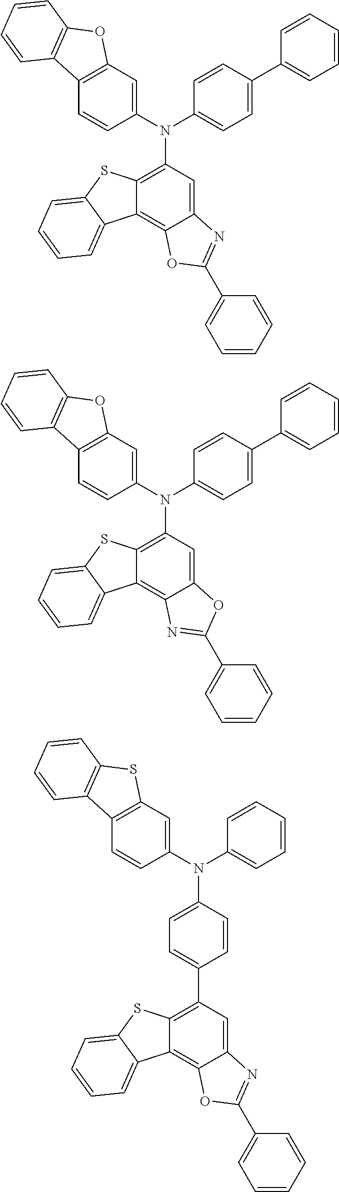 [SMILES: c1ccc(-c2ccc(N(c3ccc4c(c3)oc3ccccc34)c3cc4nc(-c5ccccc5)oc4c4c3sc3ccccc34)cc2)cc1 ; c1ccc(-c2ccc(N(c3ccc4c(c3)oc3ccccc34)c3cc4oc(-c5ccccc5)nc4c4c3sc3ccccc34)cc2)cc1 ; c1ccc(-c2nc3cc(-c4ccc(N(c5ccccc5)c5ccc6c(c5)sc5ccccc56)cc4)c4sc5ccccc5c4c3o2)cc1]

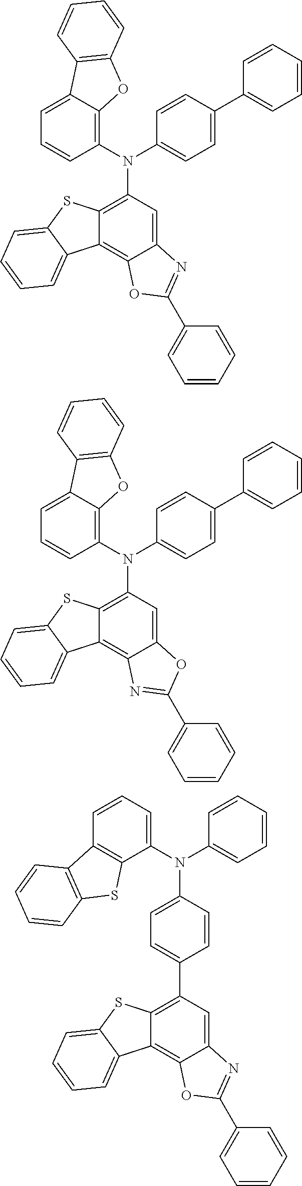

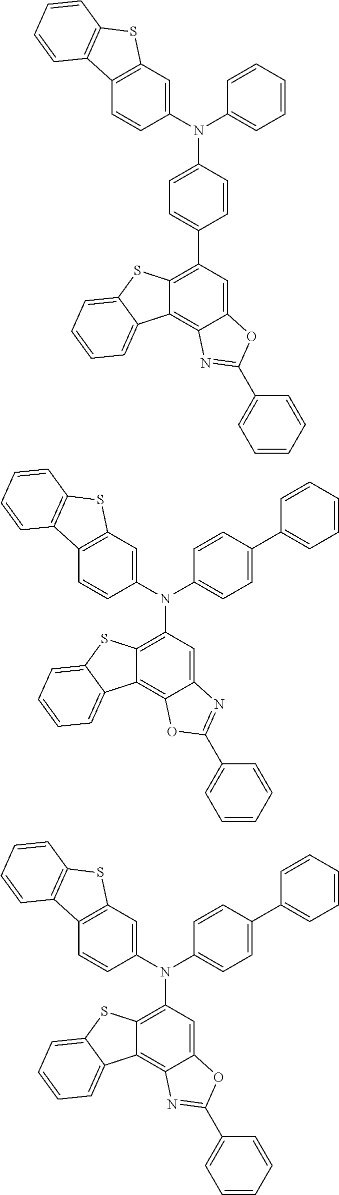

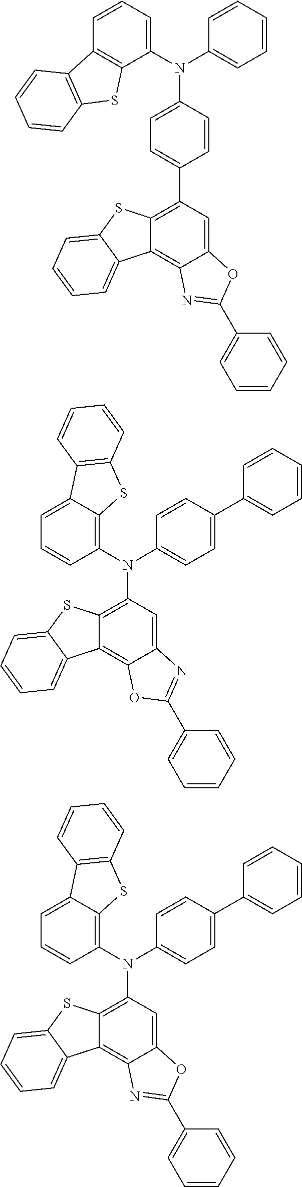 [SMILES: c1ccc(-c2nc3c(cc(-c4ccc(N(c5ccccc5)c5cccc6c5sc5ccccc56)cc4)c4sc5ccccc5c43)o2)cc1 ; c1ccc(-c2ccc(N(c3cccc4c3sc3ccccc34)c3cc4nc(-c5ccccc5)oc4c4c3sc3ccccc34)cc2)cc1 ; c1ccc(-c2ccc(N(c3cccc4c3sc3ccccc34)c3cc4oc(-c5ccccc5)nc4c4c3sc3ccccc34)cc2)cc1]

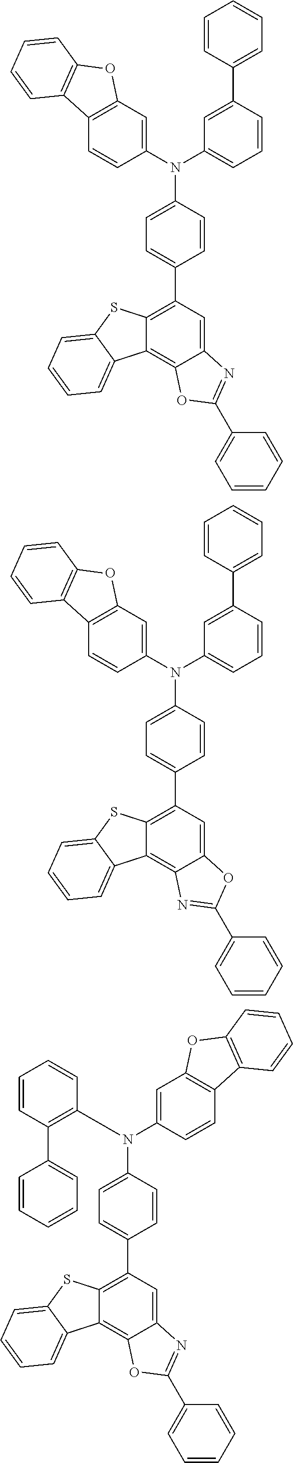

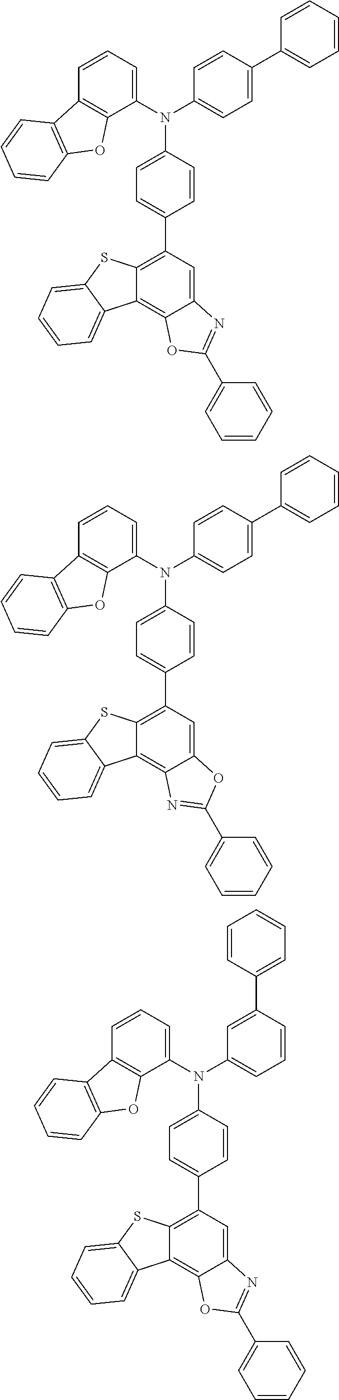

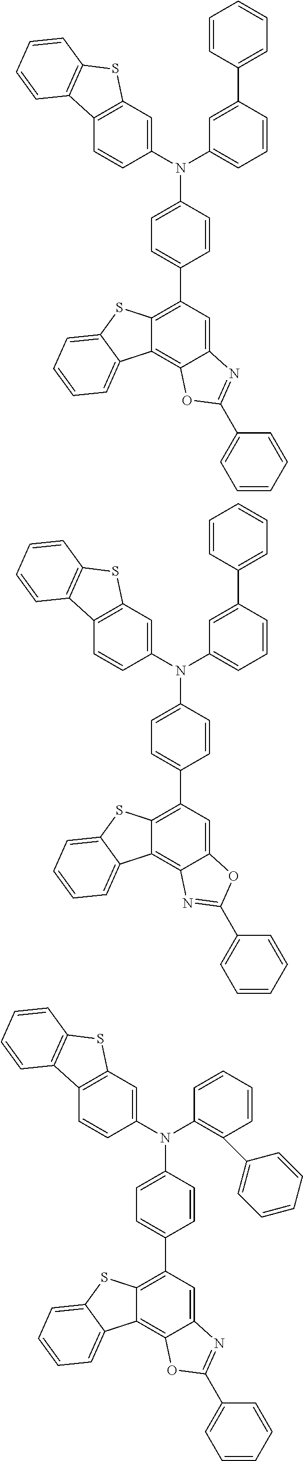

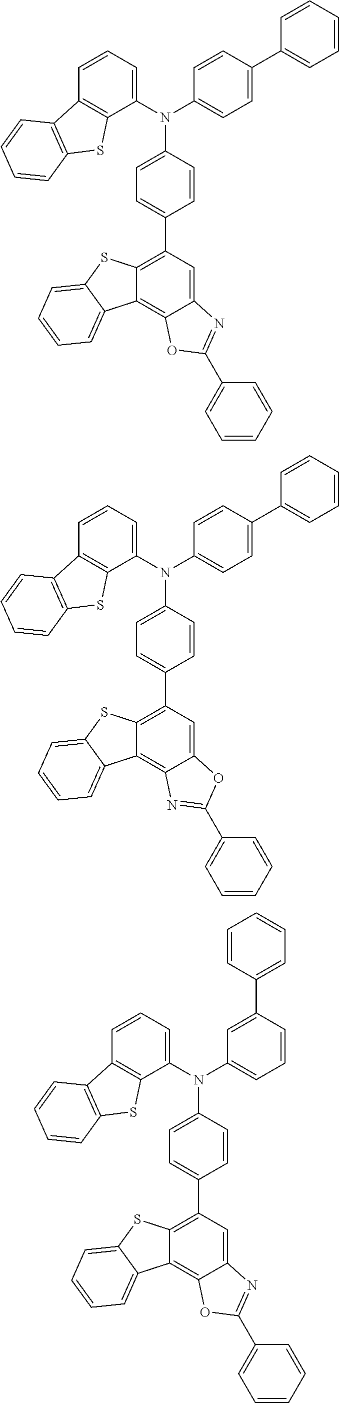

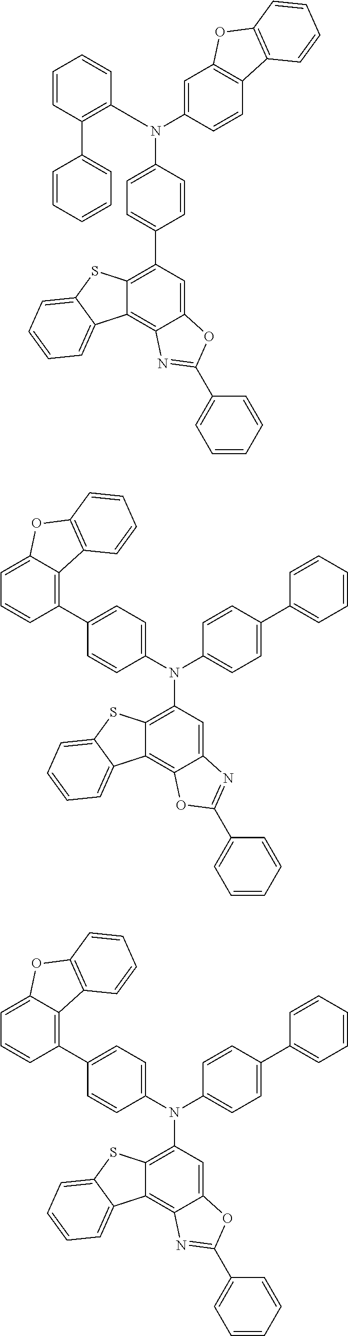 [SMILES: c1ccc(-c2nc3c(cc(-c4ccc(N(c5ccc6c(c5)oc5ccccc56)c5ccccc5-c5ccccc5)cc4)c4sc5ccccc5c43)o2)cc1 ; c1ccc(-c2ccc(N(c3ccc(-c4cccc5oc6ccccc6c45)cc3)c3cc4nc(-c5ccccc5)oc4c4c3sc3ccccc34)cc2)cc1 ; c1ccc(-c2ccc(N(c3ccc(-c4cccc5oc6ccccc6c45)cc3)c3cc4oc(-c5ccccc5)nc4c4c3sc3ccccc34)cc2)cc1]

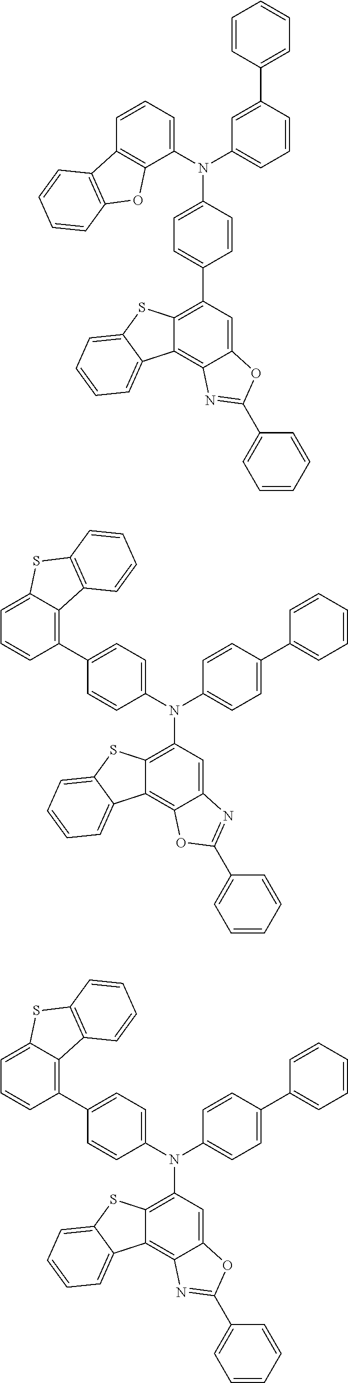 [SMILES: c1ccc(-c2cccc(N(c3ccc(-c4cc5oc(-c6ccccc6)nc5c5c4sc4ccccc45)cc3)c3cccc4c3oc3ccccc34)c2)cc1 ; c1ccc(-c2ccc(N(c3ccc(-c4cccc5sc6ccccc6c45)cc3)c3cc4nc(-c5ccccc5)oc4c4c3sc3ccccc34)cc2)cc1 ; c1ccc(-c2ccc(N(c3ccc(-c4cccc5sc6ccccc6c45)cc3)c3cc4oc(-c5ccccc5)nc4c4c3sc3ccccc34)cc2)cc1]

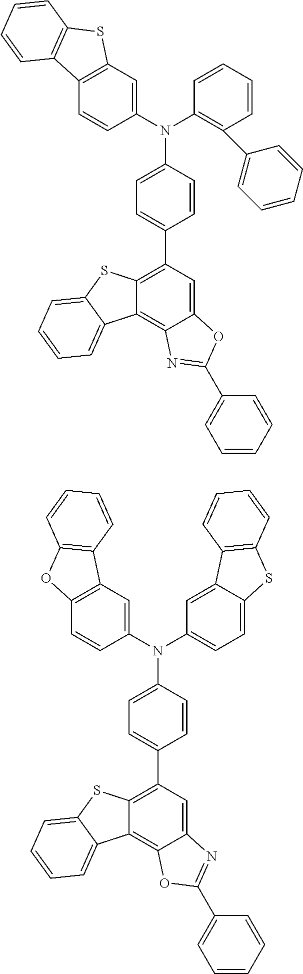

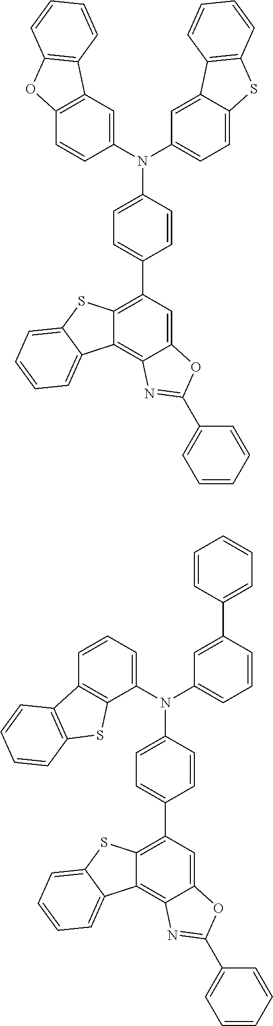 [SMILES: c1ccc(-c2nc3c(cc(-c4ccc(N(c5ccc6oc7ccccc7c6c5)c5ccc6sc7ccccc7c6c5)cc4)c4sc5ccccc5c43)o2)cc1 ; c1ccc(-c2cccc(N(c3ccc(-c4cc5oc(-c6ccccc6)nc5c5c4sc4ccccc45)cc3)c3cccc4c3sc3ccccc34)c2)cc1]

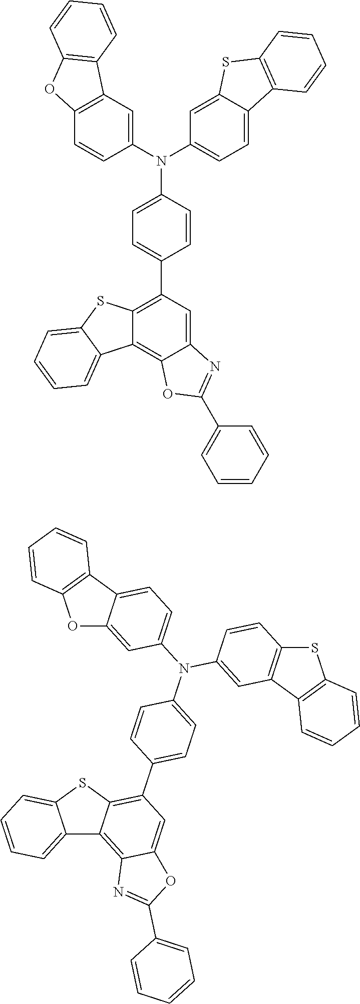 [SMILES: c1ccc(-c2nc3cc(-c4ccc(N(c5ccc6c(c5)sc5ccccc56)c5ccc6oc7ccccc7c6c5)cc4)c4sc5ccccc5c4c3o2)cc1 ; c1ccc(-c2nc3c(cc(-c4ccc(N(c5ccc6c(c5)oc5ccccc56)c5ccc6sc7ccccc7c6c5)cc4)c4sc5ccccc5c43)o2)cc1]

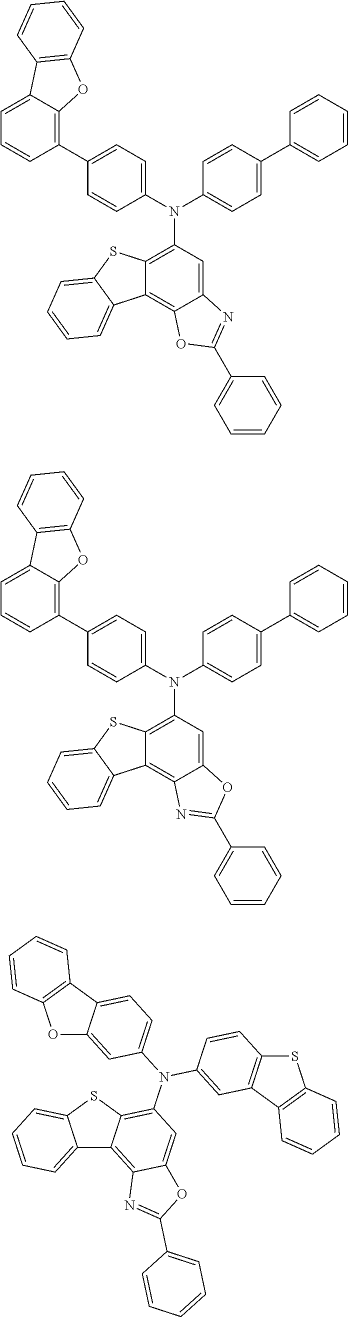 [SMILES: c1ccc(-c2ccc(N(c3ccc(-c4cccc5c4oc4ccccc45)cc3)c3cc4nc(-c5ccccc5)oc4c4c3sc3ccccc34)cc2)cc1 ; c1ccc(-c2ccc(N(c3ccc(-c4cccc5c4oc4ccccc45)cc3)c3cc4oc(-c5ccccc5)nc4c4c3sc3ccccc34)cc2)cc1 ; c1ccc(-c2nc3c(cc(N(c4ccc5c(c4)oc4ccccc45)c4ccc5sc6ccccc6c5c4)c4sc5ccccc5c43)o2)cc1]

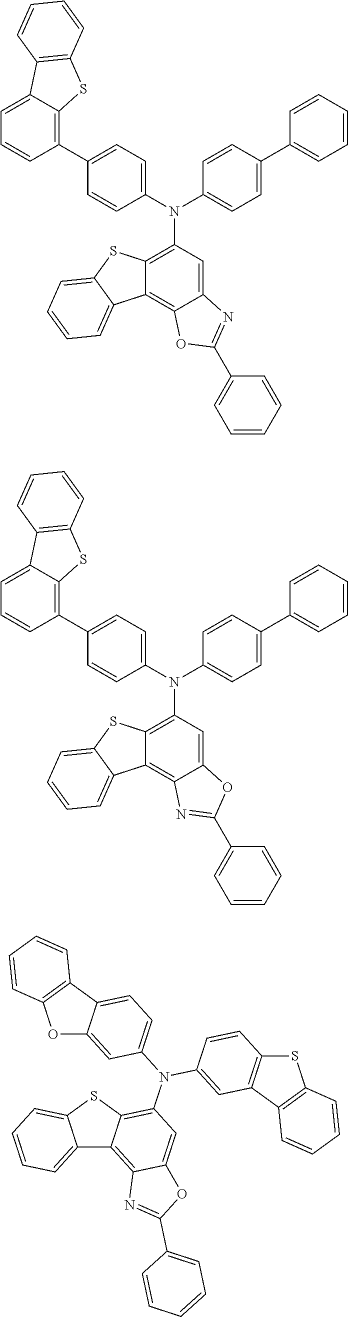 [SMILES: c1ccc(-c2ccc(N(c3ccc(-c4cccc5c4sc4ccccc45)cc3)c3cc4nc(-c5ccccc5)oc4c4c3sc3ccccc34)cc2)cc1 ; c1ccc(-c2ccc(N(c3ccc(-c4cccc5c4sc4ccccc45)cc3)c3cc4oc(-c5ccccc5)nc4c4c3sc3ccccc34)cc2)cc1 ; c1ccc(-c2nc3c(cc(N(c4ccc5c(c4)oc4ccccc45)c4ccc5sc6ccccc6c5c4)c4sc5ccccc5c43)o2)cc1]

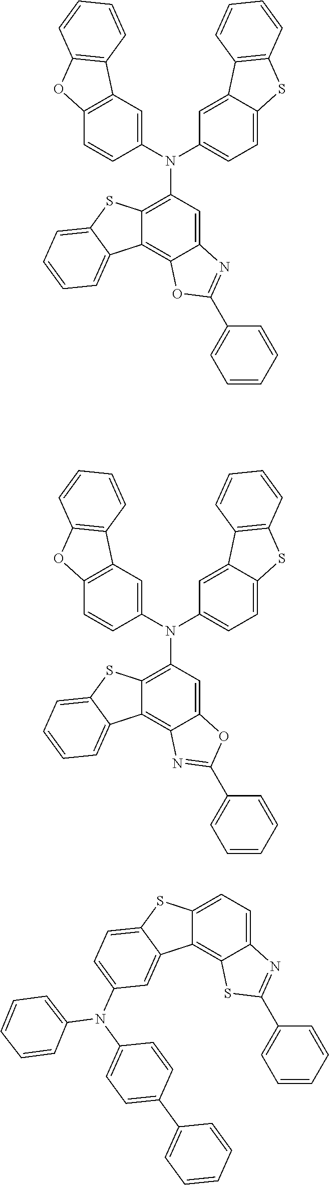

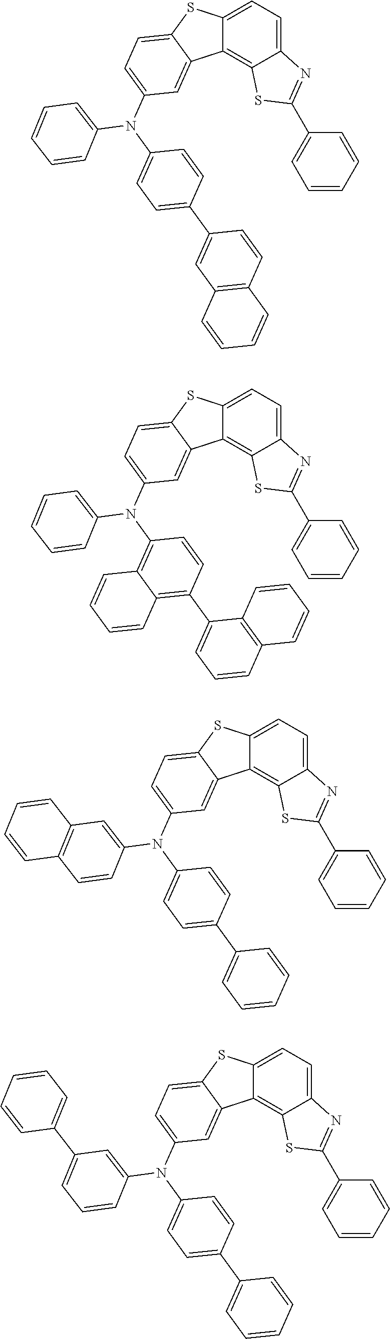

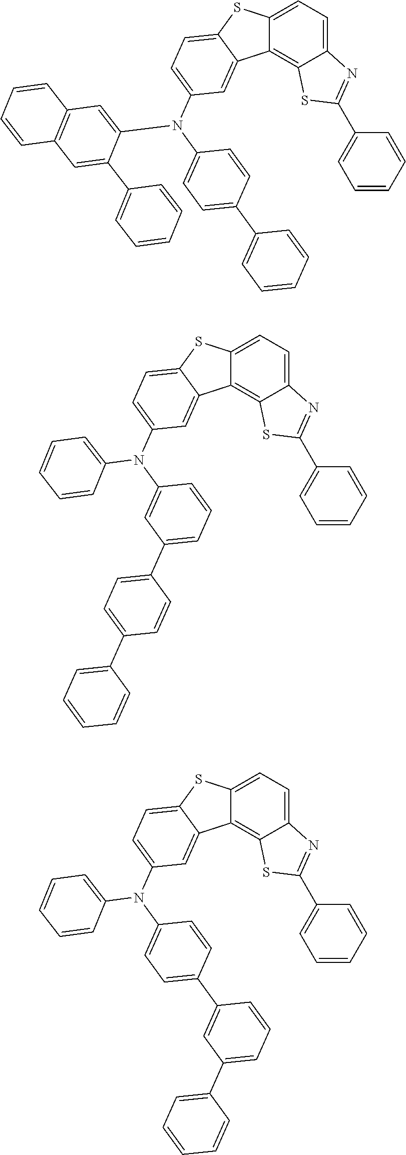

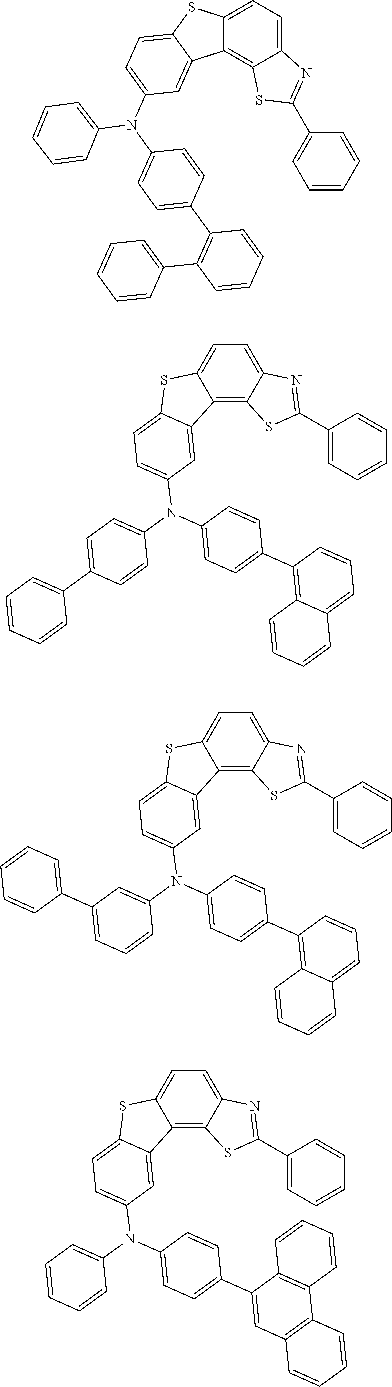 [SMILES: c1ccc(-c2nc3ccc4sc5ccc(N(c6ccccc6)c6ccc(-c7ccccc7-c7ccccc7)cc6)cc5c4c3s2)cc1 ; c1ccc(-c2ccc(N(c3ccc(-c4cccc5ccccc45)cc3)c3ccc4sc5ccc6nc(-c7ccccc7)sc6c5c4c3)cc2)cc1 ; c1ccc(-c2cccc(N(c3ccc(-c4cccc5ccccc45)cc3)c3ccc4sc5ccc6nc(-c7ccccc7)sc6c5c4c3)c2)cc1 ; c1ccc(-c2nc3ccc4sc5ccc(N(c6ccccc6)c6ccc(-c7cc8ccccc8c8ccccc78)cc6)cc5c4c3s2)cc1]

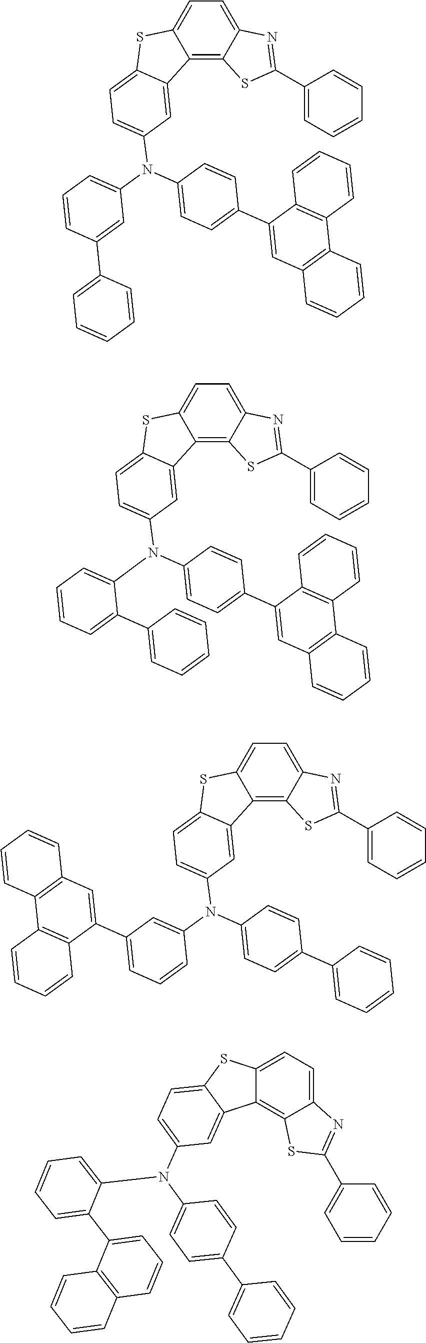 [SMILES: c1ccc(-c2cccc(N(c3ccc(-c4cc5ccccc5c5ccccc45)cc3)c3ccc4sc5ccc6nc(-c7ccccc7)sc6c5c4c3)c2)cc1 ; c1ccc(-c2nc3ccc4sc5ccc(N(c6ccc(-c7cc8ccccc8c8ccccc78)cc6)c6ccccc6-c6ccccc6)cc5c4c3s2)cc1 ; c1ccc(-c2ccc(N(c3cccc(-c4cc5ccccc5c5ccccc45)c3)c3ccc4sc5ccc6nc(-c7ccccc7)sc6c5c4c3)cc2)cc1 ; c1ccc(-c2ccc(N(c3ccc4sc5ccc6nc(-c7ccccc7)sc6c5c4c3)c3ccccc3-c3cccc4ccccc34)cc2)cc1]

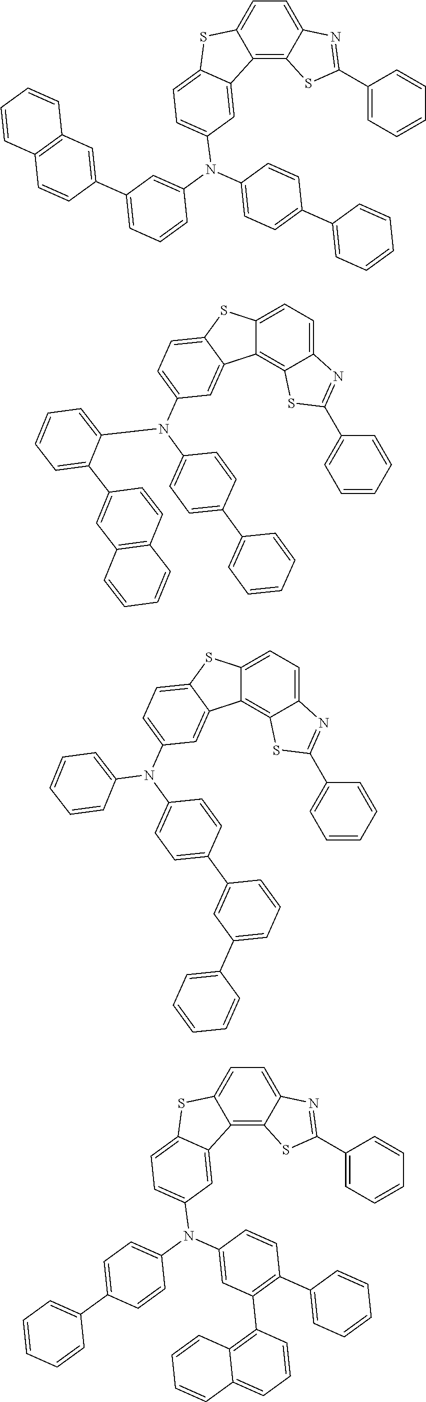

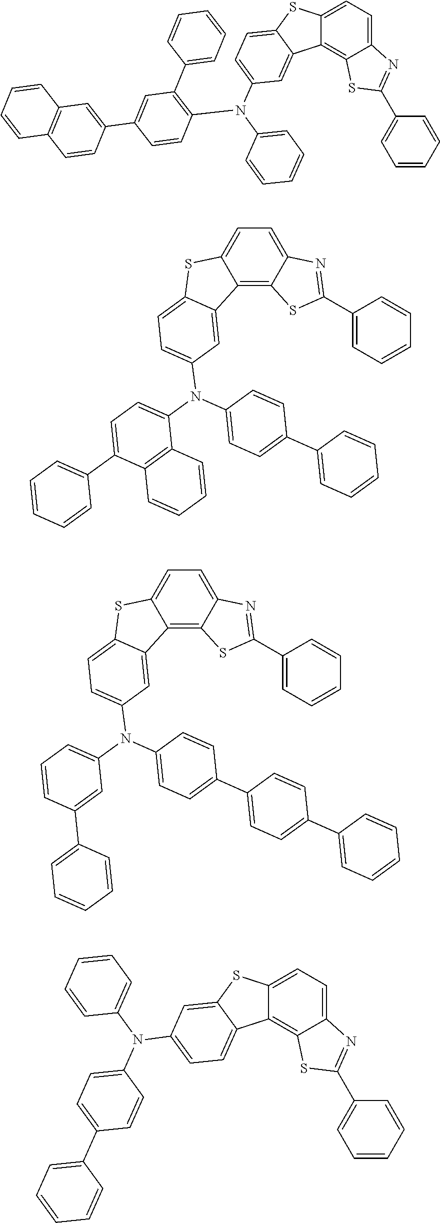

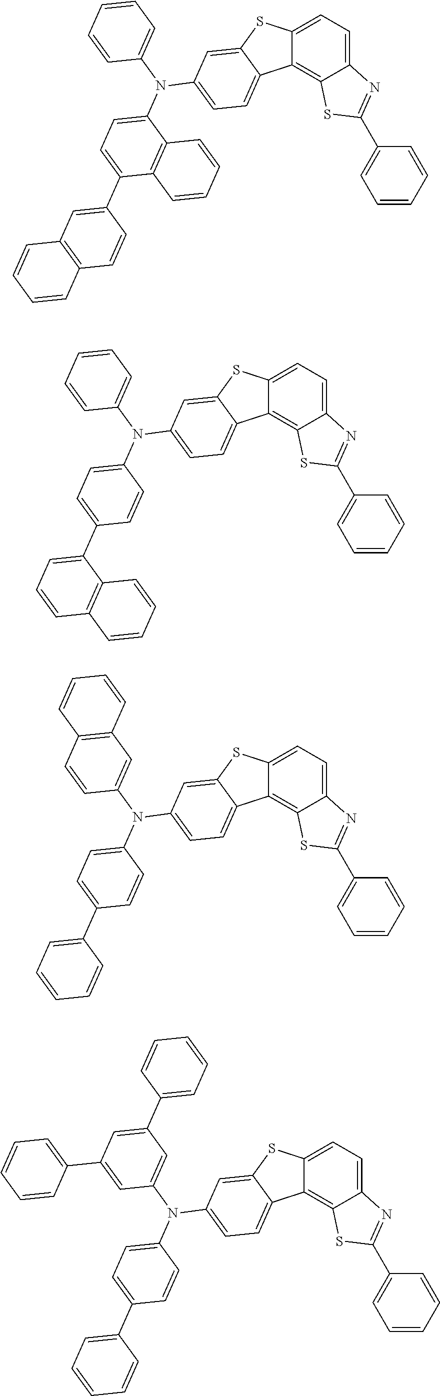

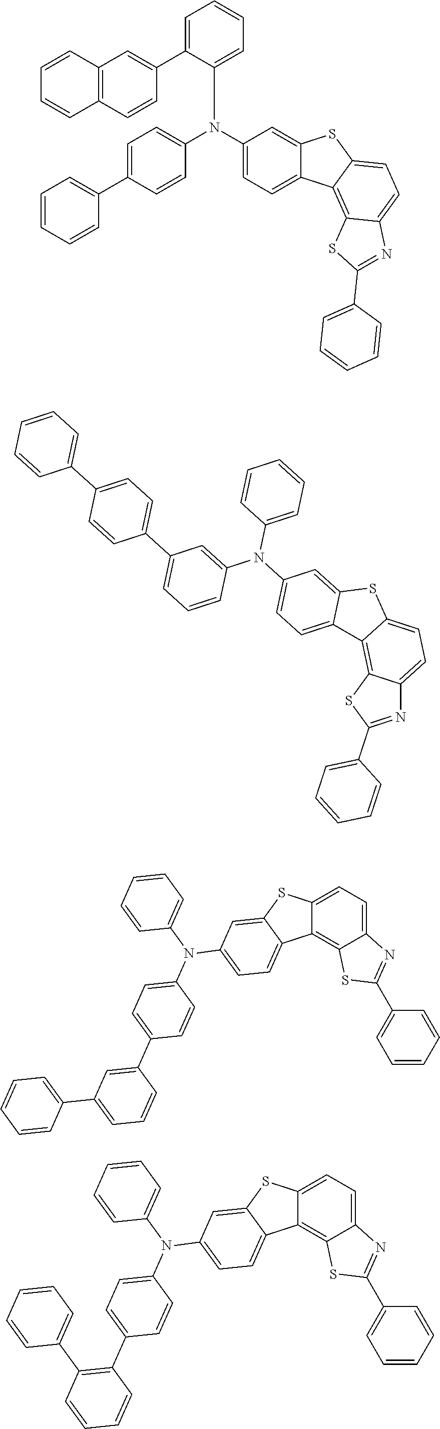

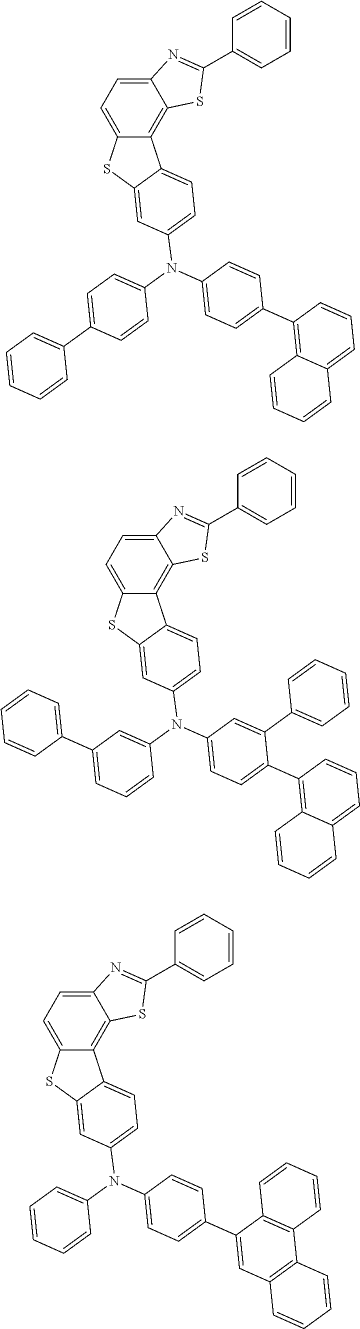 [SMILES: c1ccc(-c2ccc(N(c3ccc(-c4cccc5ccccc45)cc3)c3ccc4c(c3)sc3ccc5nc(-c6ccccc6)sc5c34)cc2)cc1 ; c1ccc(-c2cccc(N(c3ccc(-c4cccc5ccccc45)c(-c4ccccc4)c3)c3ccc4c(c3)sc3ccc5nc(-c6ccccc6)sc5c34)c2)cc1 ; c1ccc(-c2nc3ccc4sc5cc(N(c6ccccc6)c6ccc(-c7cc8ccccc8c8ccccc78)cc6)ccc5c4c3s2)cc1]

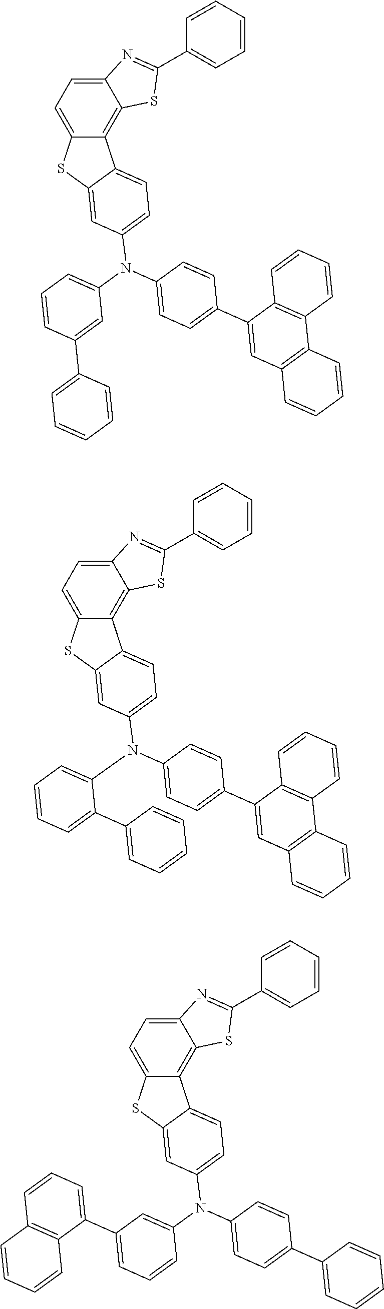 [SMILES: c1ccc(-c2cccc(N(c3ccc(-c4cc5ccccc5c5ccccc45)cc3)c3ccc4c(c3)sc3ccc5nc(-c6ccccc6)sc5c34)c2)cc1 ; c1ccc(-c2nc3ccc4sc5cc(N(c6ccc(-c7cc8ccccc8c8ccccc78)cc6)c6ccccc6-c6ccccc6)ccc5c4c3s2)cc1 ; c1ccc(-c2ccc(N(c3cccc(-c4cccc5ccccc45)c3)c3ccc4c(c3)sc3ccc5nc(-c6ccccc6)sc5c34)cc2)cc1]

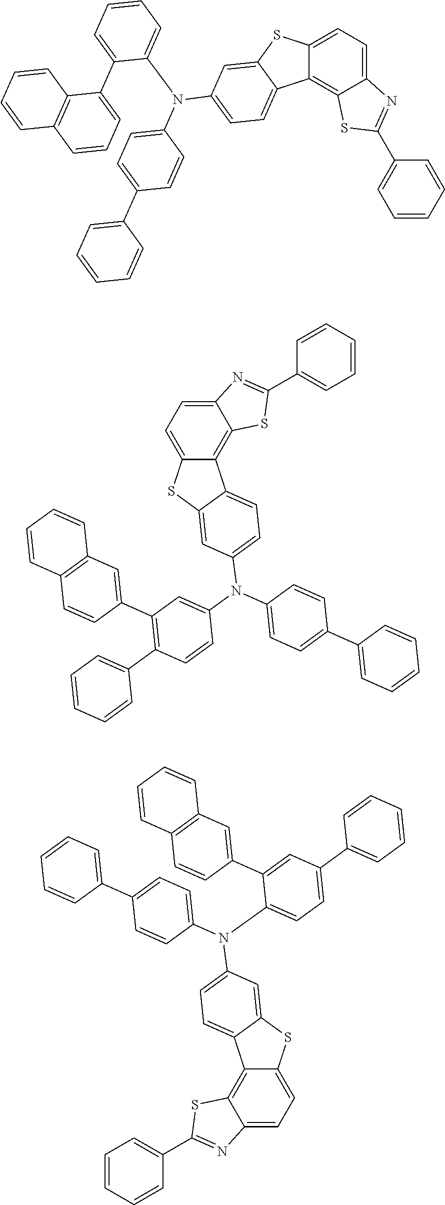 [SMILES: c1ccc(-c2ccc(N(c3ccc4c(c3)sc3ccc5nc(-c6ccccc6)sc5c34)c3ccccc3-c3cccc4ccccc34)cc2)cc1 ; c1ccc(-c2ccc(N(c3ccc(-c4ccccc4)c(-c4ccc5ccccc5c4)c3)c3ccc4c(c3)sc3ccc5nc(-c6ccccc6)sc5c34)cc2)cc1 ; c1ccc(-c2ccc(N(c3ccc4c(c3)sc3ccc5nc(-c6ccccc6)sc5c34)c3ccc(-c4ccccc4)cc3-c3ccc4ccccc4c3)cc2)cc1]

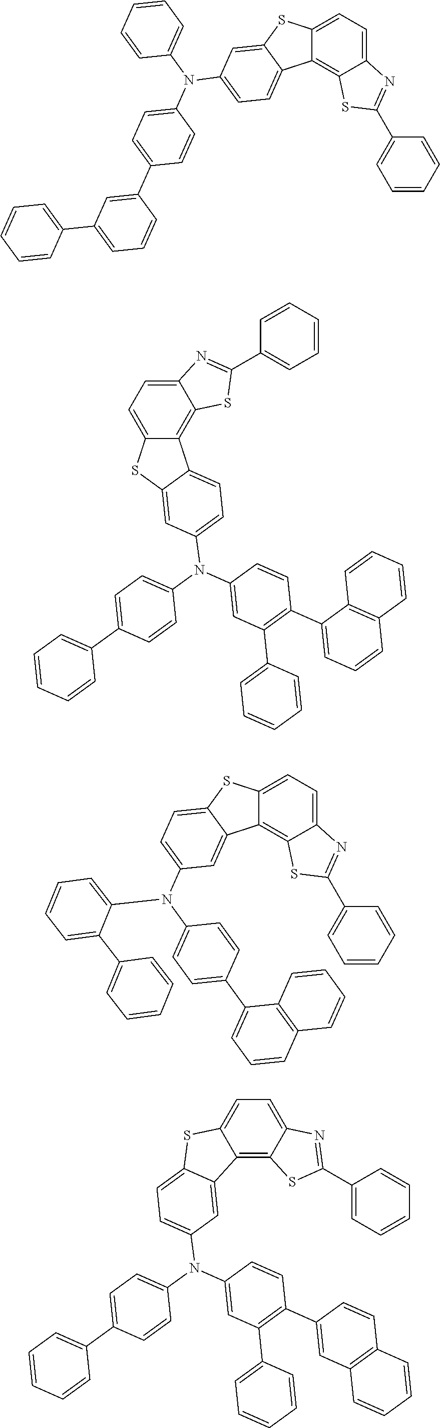

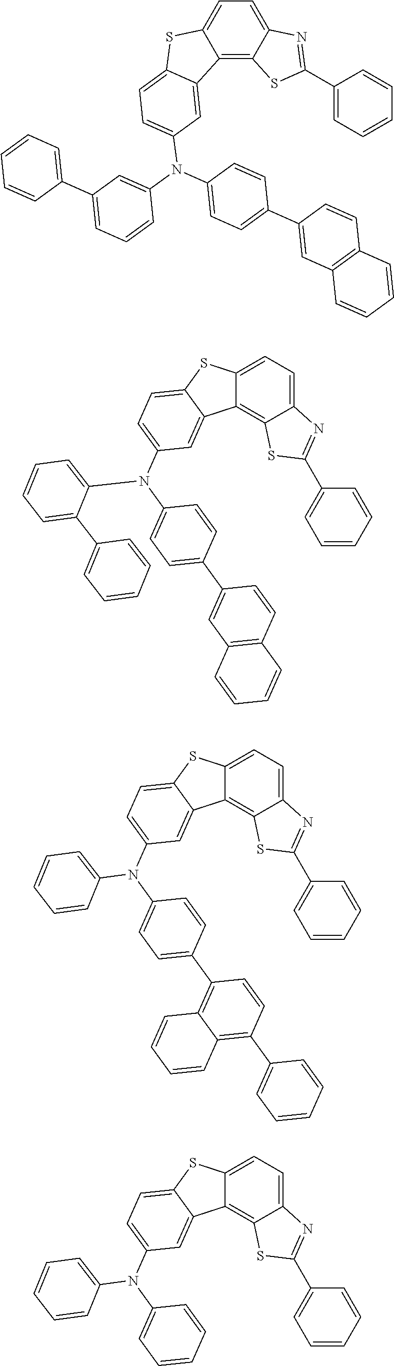

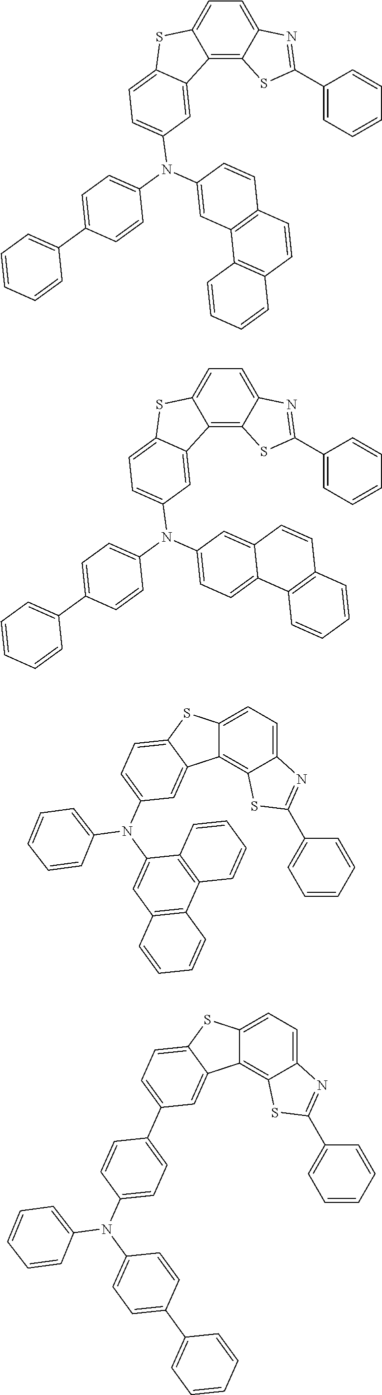

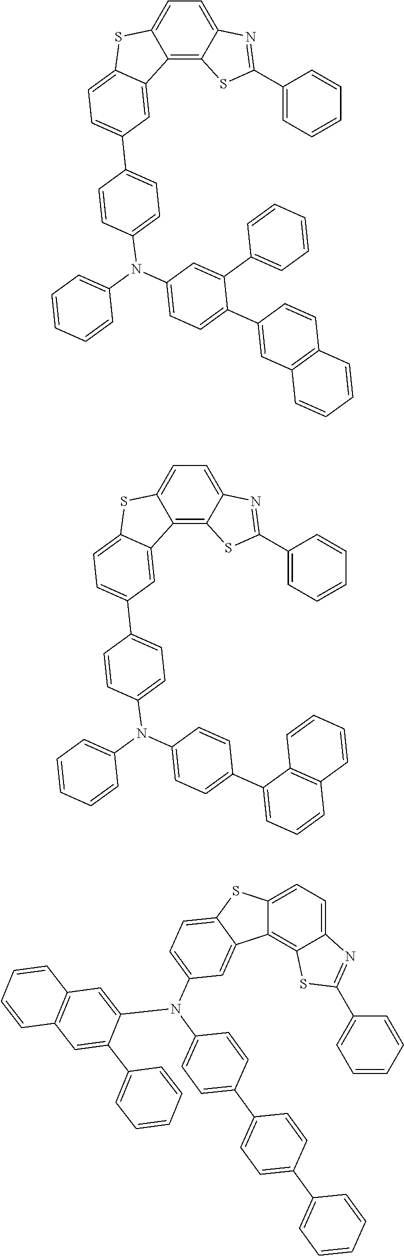 [SMILES: c1ccc(-c2nc3ccc4sc5ccc(-c6ccc(N(c7ccccc7)c7ccc(-c8ccc9ccccc9c8)c(-c8ccccc8)c7)cc6)cc5c4c3s2)cc1 ; c1ccc(-c2nc3ccc4sc5ccc(-c6ccc(N(c7ccccc7)c7ccc(-c8cccc9ccccc89)cc7)cc6)cc5c4c3s2)cc1 ; c1ccc(-c2ccc(-c3ccc(N(c4ccc5sc6ccc7nc(-c8ccccc8)sc7c6c5c4)c4cc5ccccc5cc4-c4ccccc4)cc3)cc2)cc1]

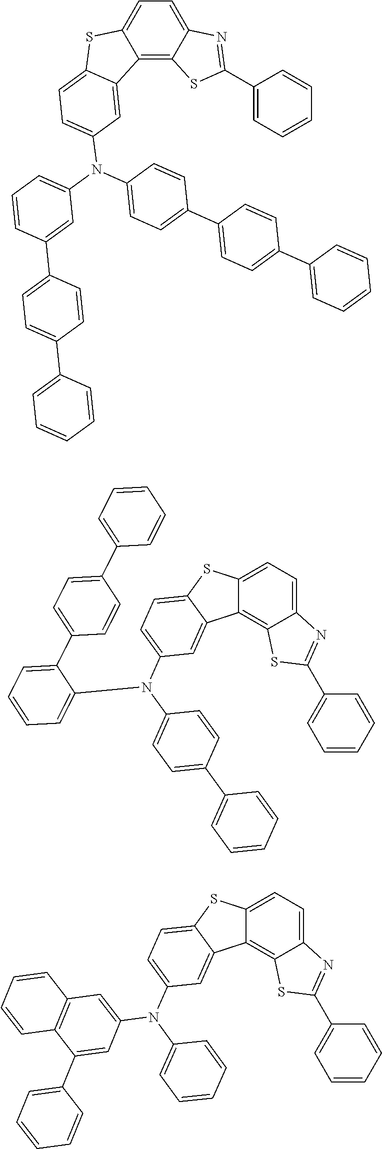

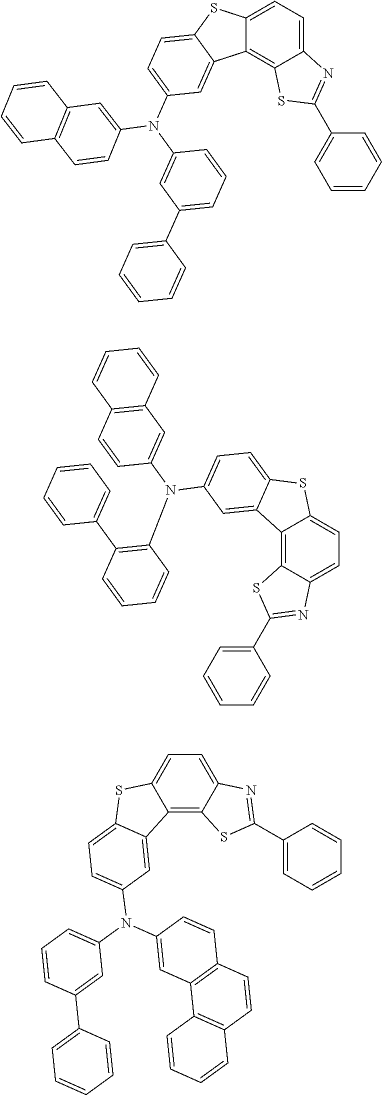

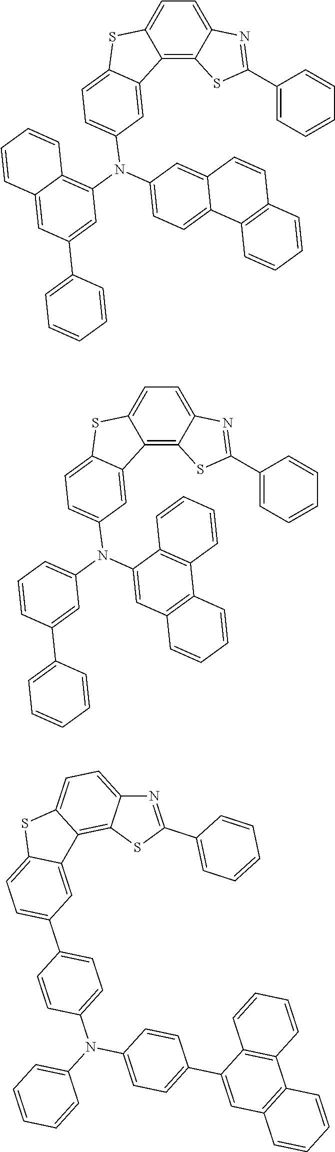 [SMILES: c1ccc(-c2cc(N(c3ccc4c(ccc5ccccc54)c3)c3ccc4sc5ccc6nc(-c7ccccc7)sc6c5c4c3)c3ccccc3c2)cc1 ; c1ccc(-c2cccc(N(c3ccc4sc5ccc6nc(-c7ccccc7)sc6c5c4c3)c3cc4ccccc4c4ccccc34)c2)cc1 ; c1ccc(-c2nc3ccc4sc5ccc(-c6ccc(N(c7ccccc7)c7ccc(-c8cc9ccccc9c9ccccc89)cc7)cc6)cc5c4c3s2)cc1]

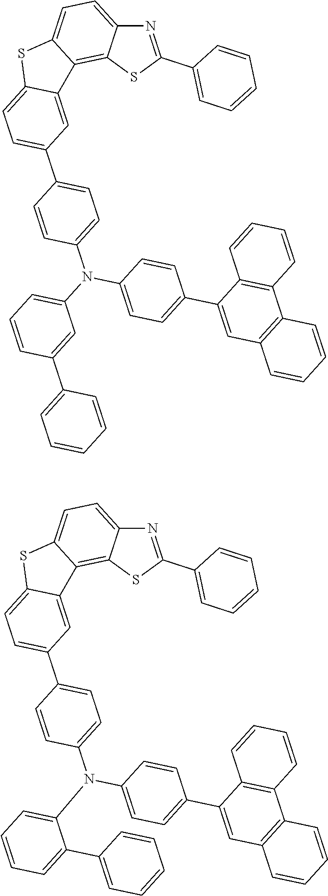 [SMILES: c1ccc(-c2cccc(N(c3ccc(-c4ccc5sc6ccc7nc(-c8ccccc8)sc7c6c5c4)cc3)c3ccc(-c4cc5ccccc5c5ccccc45)cc3)c2)cc1 ; c1ccc(-c2nc3ccc4sc5ccc(-c6ccc(N(c7ccc(-c8cc9ccccc9c9ccccc89)cc7)c7ccccc7-c7ccccc7)cc6)cc5c4c3s2)cc1]

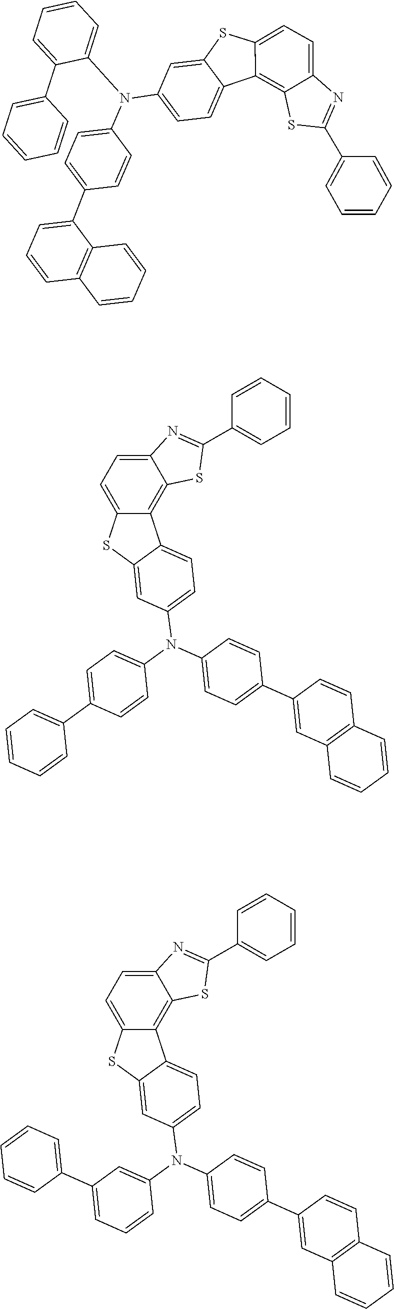 [SMILES: c1ccc(-c2nc3ccc4sc5cc(N(c6ccc(-c7cccc8ccccc78)cc6)c6ccccc6-c6ccccc6)ccc5c4c3s2)cc1 ; c1ccc(-c2ccc(N(c3ccc(-c4ccc5ccccc5c4)cc3)c3ccc4c(c3)sc3ccc5nc(-c6ccccc6)sc5c34)cc2)cc1 ; c1ccc(-c2cccc(N(c3ccc(-c4ccc5ccccc5c4)cc3)c3ccc4c(c3)sc3ccc5nc(-c6ccccc6)sc5c34)c2)cc1]

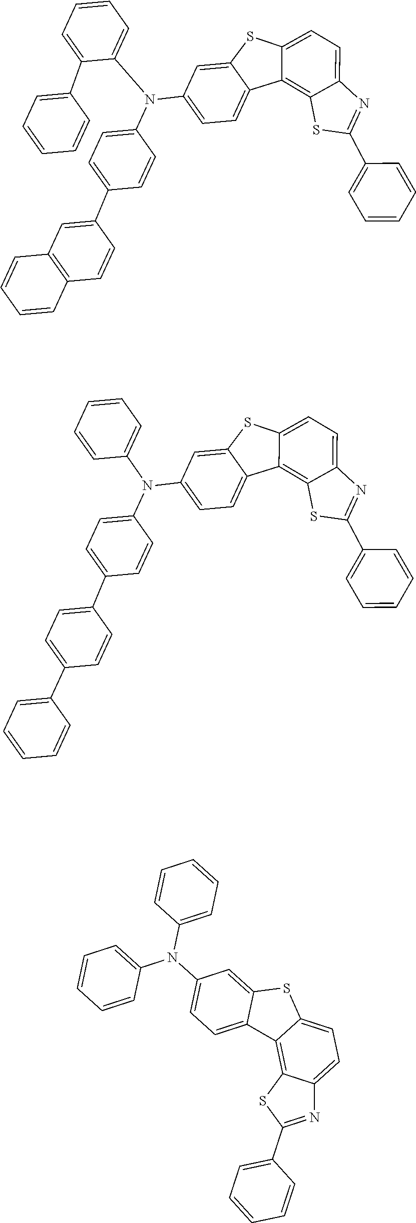 [SMILES: c1ccc(-c2nc3ccc4sc5cc(N(c6ccc(-c7ccc8ccccc8c7)cc6)c6ccccc6-c6ccccc6)ccc5c4c3s2)cc1 ; c1ccc(-c2ccc(-c3ccc(N(c4ccccc4)c4ccc5c(c4)sc4ccc6nc(-c7ccccc7)sc6c45)cc3)cc2)cc1 ; c1ccc(-c2nc3ccc4sc5cc(N(c6ccccc6)c6ccccc6)ccc5c4c3s2)cc1]

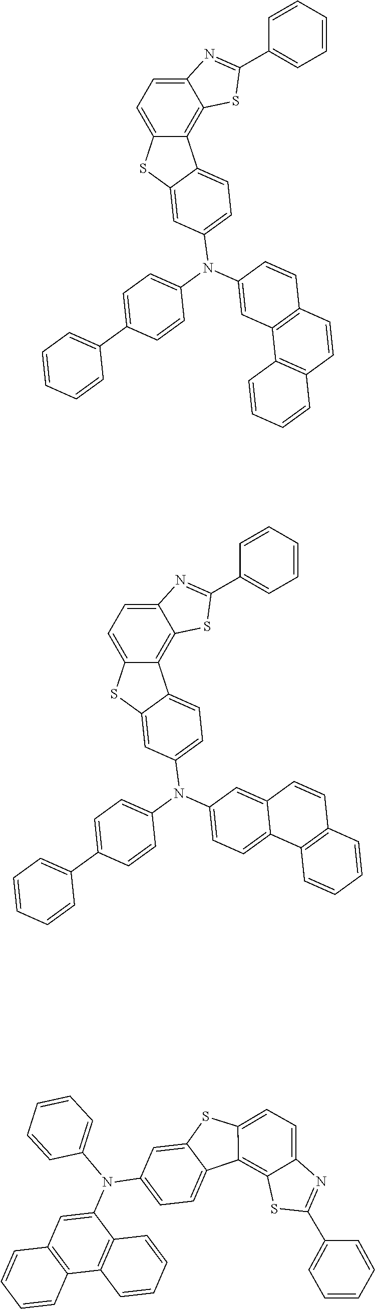 [SMILES: c1ccc(-c2ccc(N(c3ccc4c(c3)sc3ccc5nc(-c6ccccc6)sc5c34)c3ccc4ccc5ccccc5c4c3)cc2)cc1 ; c1ccc(-c2ccc(N(c3ccc4c(ccc5ccccc54)c3)c3ccc4c(c3)sc3ccc5nc(-c6ccccc6)sc5c34)cc2)cc1 ; c1ccc(-c2nc3ccc4sc5cc(N(c6ccccc6)c6cc7ccccc7c7ccccc67)ccc5c4c3s2)cc1]

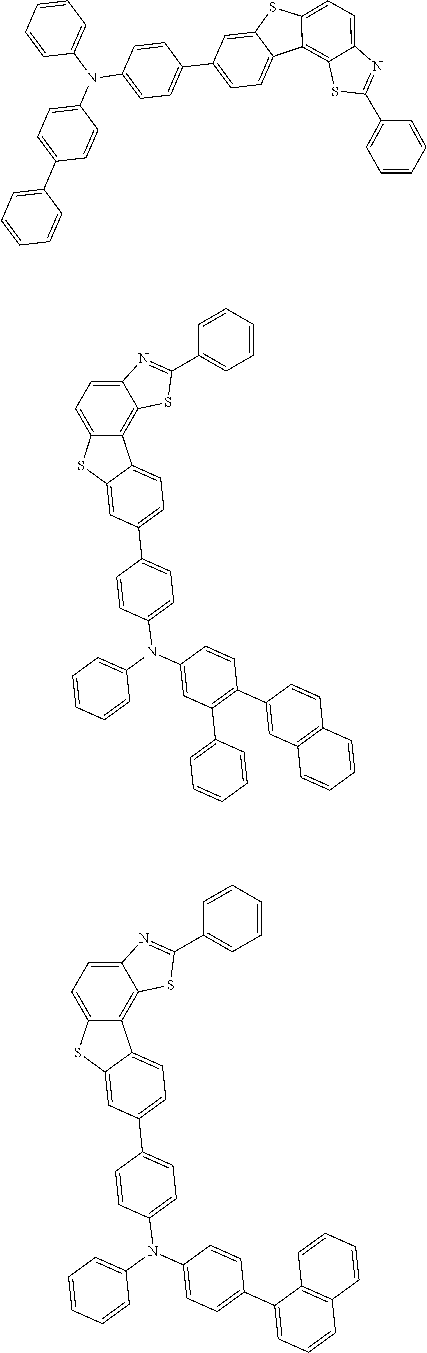 [SMILES: c1ccc(-c2ccc(N(c3ccccc3)c3ccc(-c4ccc5c(c4)sc4ccc6nc(-c7ccccc7)sc6c45)cc3)cc2)cc1 ; c1ccc(-c2nc3ccc4sc5cc(-c6ccc(N(c7ccccc7)c7ccc(-c8ccc9ccccc9c8)c(-c8ccccc8)c7)cc6)ccc5c4c3s2)cc1 ; c1ccc(-c2nc3ccc4sc5cc(-c6ccc(N(c7ccccc7)c7ccc(-c8cccc9ccccc89)cc7)cc6)ccc5c4c3s2)cc1]

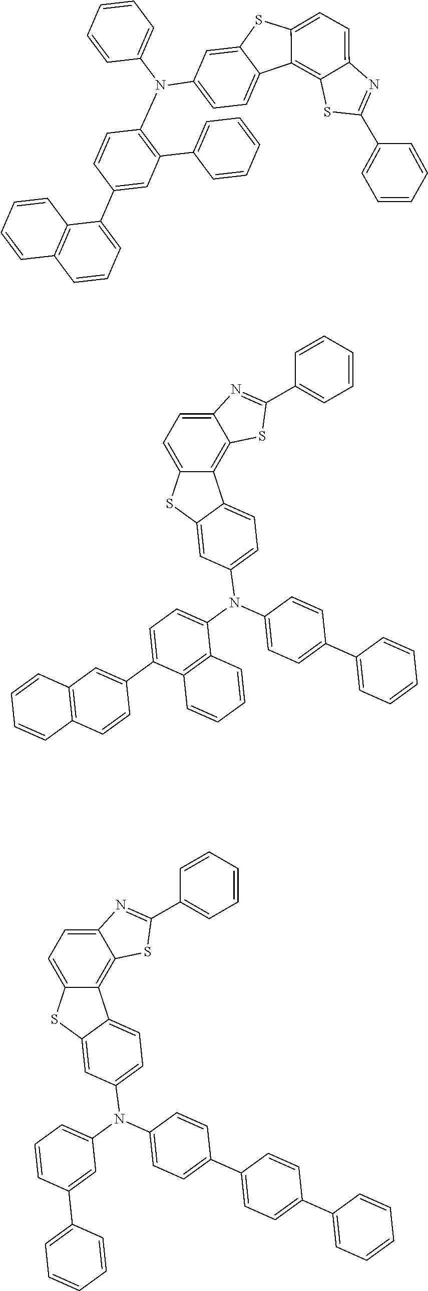 [SMILES: c1ccc(-c2nc3ccc4sc5cc(N(c6ccccc6)c6ccc(-c7cccc8ccccc78)cc6-c6ccccc6)ccc5c4c3s2)cc1 ; c1ccc(-c2ccc(N(c3ccc4c(c3)sc3ccc5nc(-c6ccccc6)sc5c34)c3ccc(-c4ccc5ccccc5c4)c4ccccc34)cc2)cc1 ; c1ccc(-c2ccc(-c3ccc(N(c4cccc(-c5ccccc5)c4)c4ccc5c(c4)sc4ccc6nc(-c7ccccc7)sc6c45)cc3)cc2)cc1]

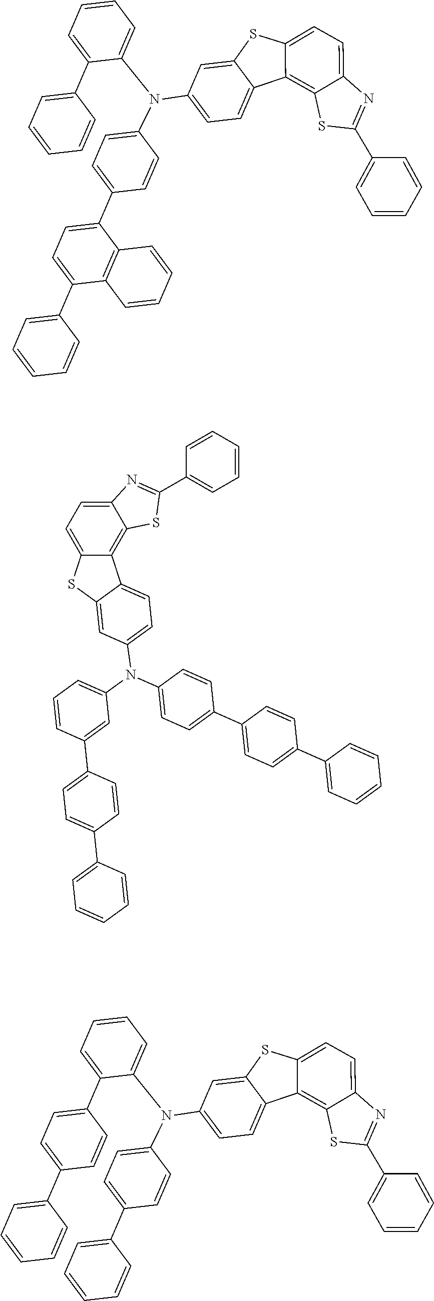 [SMILES: c1ccc(-c2nc3ccc4sc5cc(N(c6ccc(-c7ccc(-c8ccccc8)c8ccccc78)cc6)c6ccccc6-c6ccccc6)ccc5c4c3s2)cc1 ; c1ccc(-c2ccc(-c3ccc(N(c4cccc(-c5ccc(-c6ccccc6)cc5)c4)c4ccc5c(c4)sc4ccc6nc(-c7ccccc7)sc6c45)cc3)cc2)cc1 ; c1ccc(-c2ccc(-c3ccccc3N(c3ccc(-c4ccccc4)cc3)c3ccc4c(c3)sc3ccc5nc(-c6ccccc6)sc5c34)cc2)cc1]

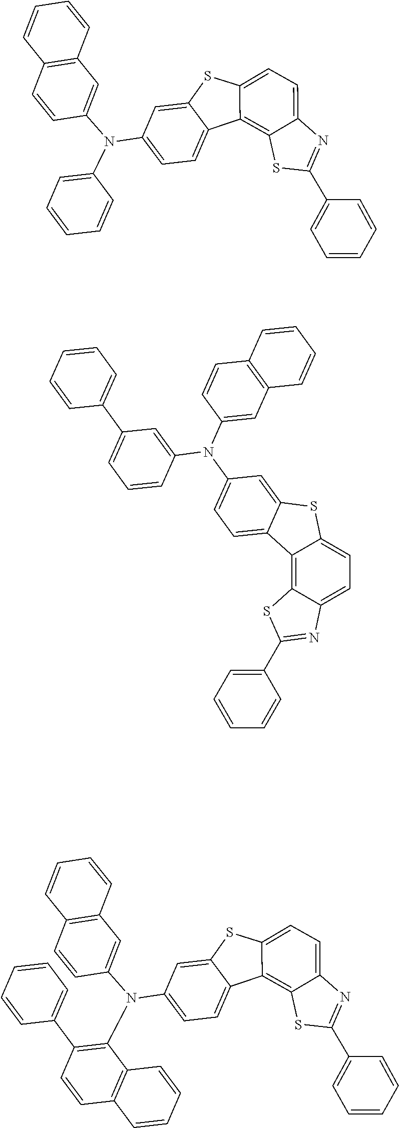 [SMILES: c1ccc(-c2nc3ccc4sc5cc(N(c6ccccc6)c6ccc7ccccc7c6)ccc5c4c3s2)cc1 ; c1ccc(-c2cccc(N(c3ccc4ccccc4c3)c3ccc4c(c3)sc3ccc5nc(-c6ccccc6)sc5c34)c2)cc1 ; c1ccc(-c2nc3ccc4sc5cc(N(c6ccc7ccccc7c6)c6c(-c7ccccc7)ccc7ccccc67)ccc5c4c3s2)cc1]

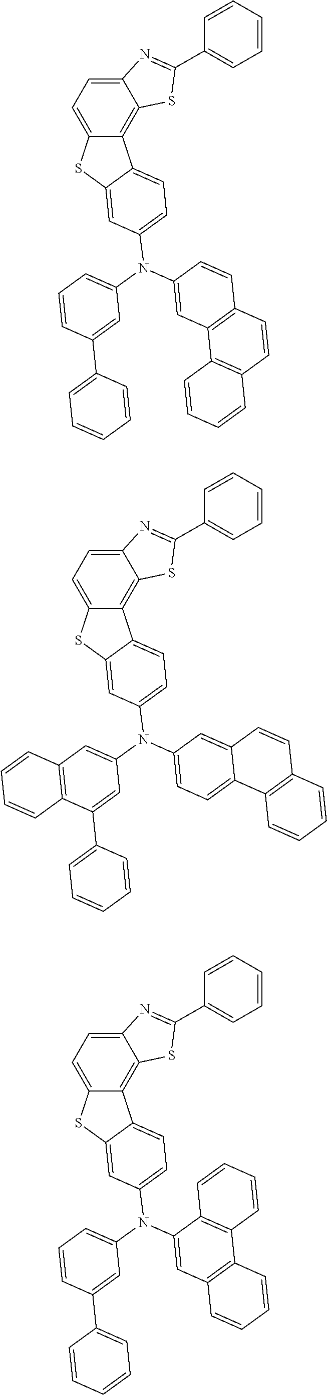 [SMILES: c1ccc(-c2cccc(N(c3ccc4c(c3)sc3ccc5nc(-c6ccccc6)sc5c34)c3ccc4ccc5ccccc5c4c3)c2)cc1 ; c1ccc(-c2nc3ccc4sc5cc(N(c6cc(-c7ccccc7)c7ccccc7c6)c6ccc7c(ccc8ccccc87)c6)ccc5c4c3s2)cc1 ; c1ccc(-c2cccc(N(c3ccc4c(c3)sc3ccc5nc(-c6ccccc6)sc5c34)c3cc4ccccc4c4ccccc34)c2)cc1]

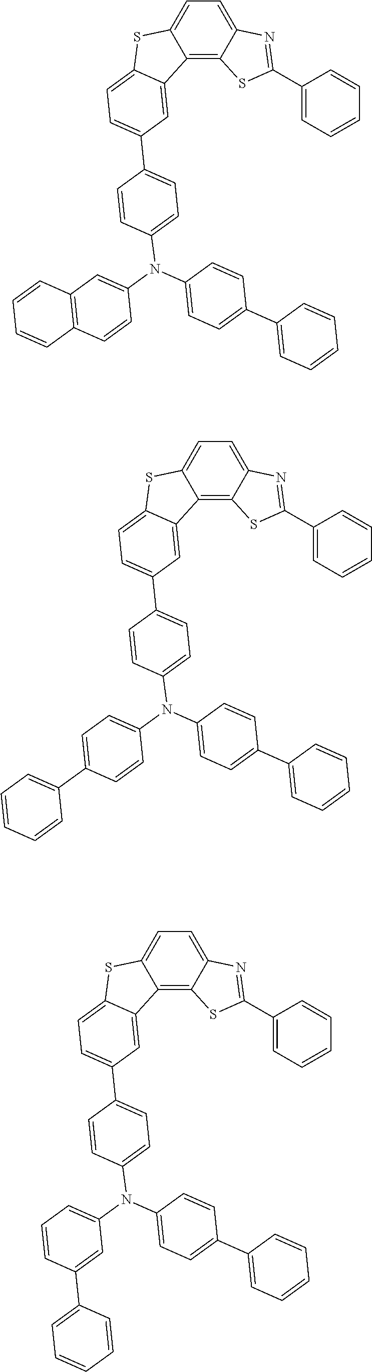 [SMILES: c1ccc(-c2ccc(N(c3ccc(-c4ccc5sc6ccc7nc(-c8ccccc8)sc7c6c5c4)cc3)c3ccc4ccccc4c3)cc2)cc1 ; c1ccc(-c2ccc(N(c3ccc(-c4ccccc4)cc3)c3ccc(-c4ccc5sc6ccc7nc(-c8ccccc8)sc7c6c5c4)cc3)cc2)cc1 ; c1ccc(-c2ccc(N(c3ccc(-c4ccc5sc6ccc7nc(-c8ccccc8)sc7c6c5c4)cc3)c3cccc(-c4ccccc4)c3)cc2)cc1]

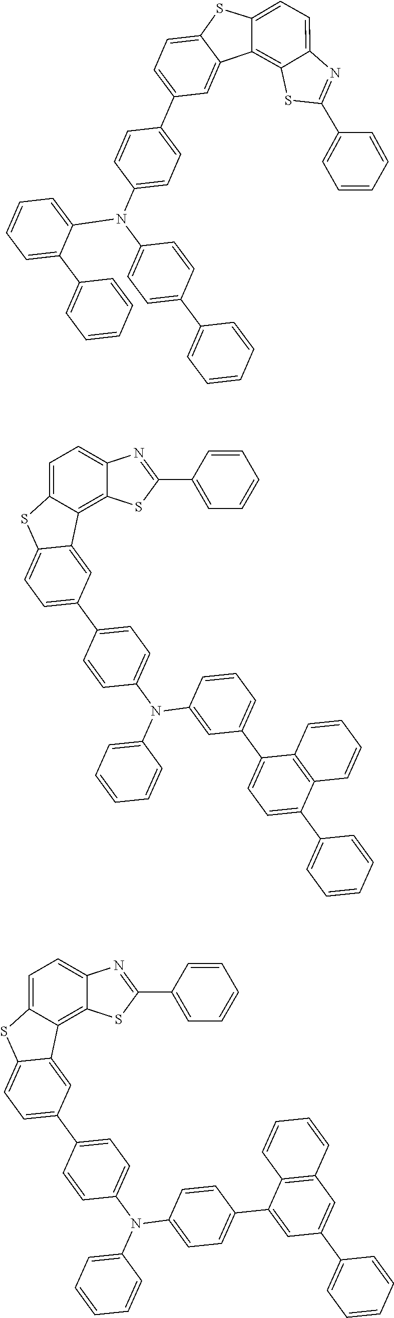 [SMILES: c1ccc(-c2ccc(N(c3ccc(-c4ccc5sc6ccc7nc(-c8ccccc8)sc7c6c5c4)cc3)c3ccccc3-c3ccccc3)cc2)cc1 ; c1ccc(-c2nc3ccc4sc5ccc(-c6ccc(N(c7ccccc7)c7cccc(-c8ccc(-c9ccccc9)c9ccccc89)c7)cc6)cc5c4c3s2)cc1 ; c1ccc(-c2cc(-c3ccc(N(c4ccccc4)c4ccc(-c5ccc6sc7ccc8nc(-c9ccccc9)sc8c7c6c5)cc4)cc3)c3ccccc3c2)cc1]

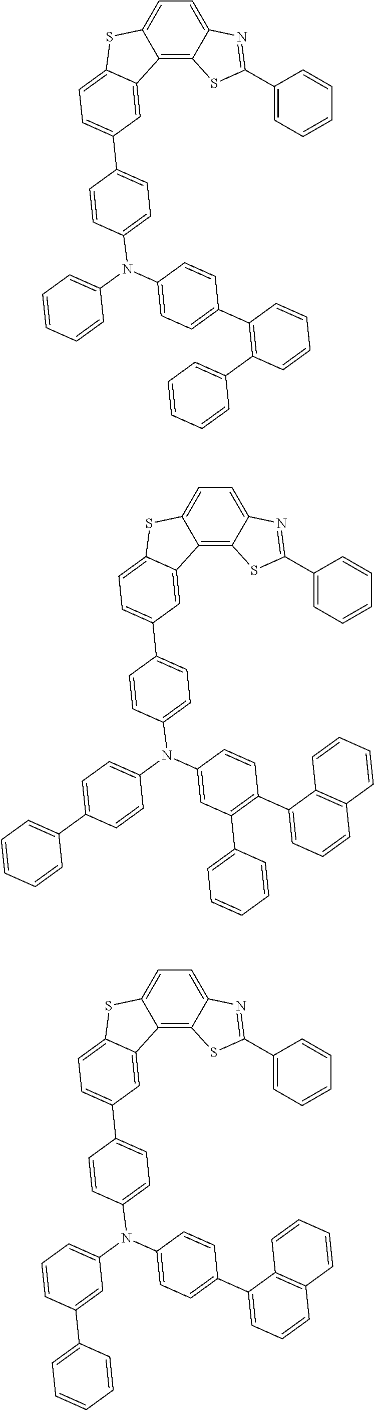 [SMILES: c1ccc(-c2nc3ccc4sc5ccc(-c6ccc(N(c7ccccc7)c7ccc(-c8ccccc8-c8ccccc8)cc7)cc6)cc5c4c3s2)cc1 ; c1ccc(-c2ccc(N(c3ccc(-c4ccc5sc6ccc7nc(-c8ccccc8)sc7c6c5c4)cc3)c3ccc(-c4cccc5ccccc45)c(-c4ccccc4)c3)cc2)cc1 ; c1ccc(-c2cccc(N(c3ccc(-c4ccc5sc6ccc7nc(-c8ccccc8)sc7c6c5c4)cc3)c3ccc(-c4cccc5ccccc45)cc3)c2)cc1]

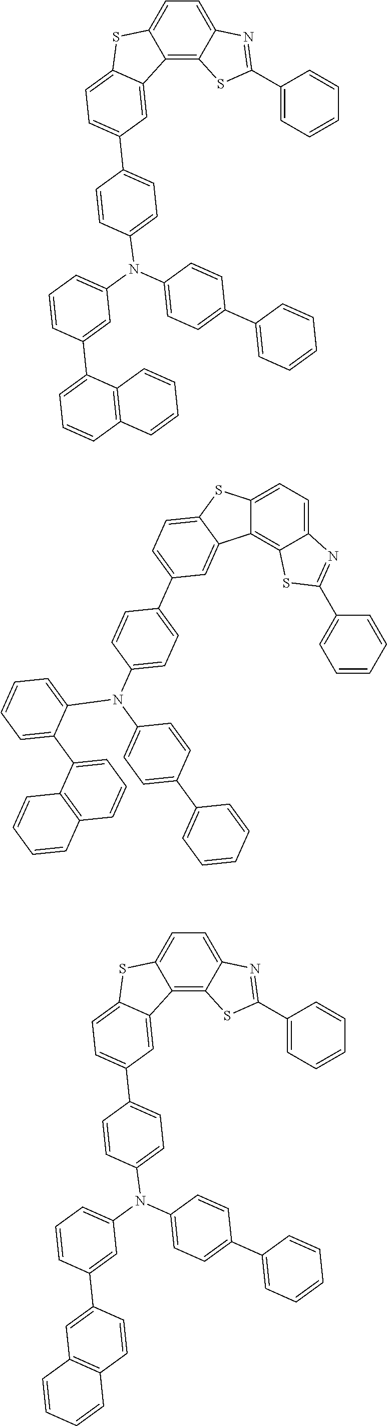 [SMILES: c1ccc(-c2ccc(N(c3ccc(-c4ccc5sc6ccc7nc(-c8ccccc8)sc7c6c5c4)cc3)c3cccc(-c4cccc5ccccc45)c3)cc2)cc1 ; c1ccc(-c2ccc(N(c3ccc(-c4ccc5sc6ccc7nc(-c8ccccc8)sc7c6c5c4)cc3)c3ccccc3-c3cccc4ccccc34)cc2)cc1 ; c1ccc(-c2ccc(N(c3ccc(-c4ccc5sc6ccc7nc(-c8ccccc8)sc7c6c5c4)cc3)c3cccc(-c4ccc5ccccc5c4)c3)cc2)cc1]

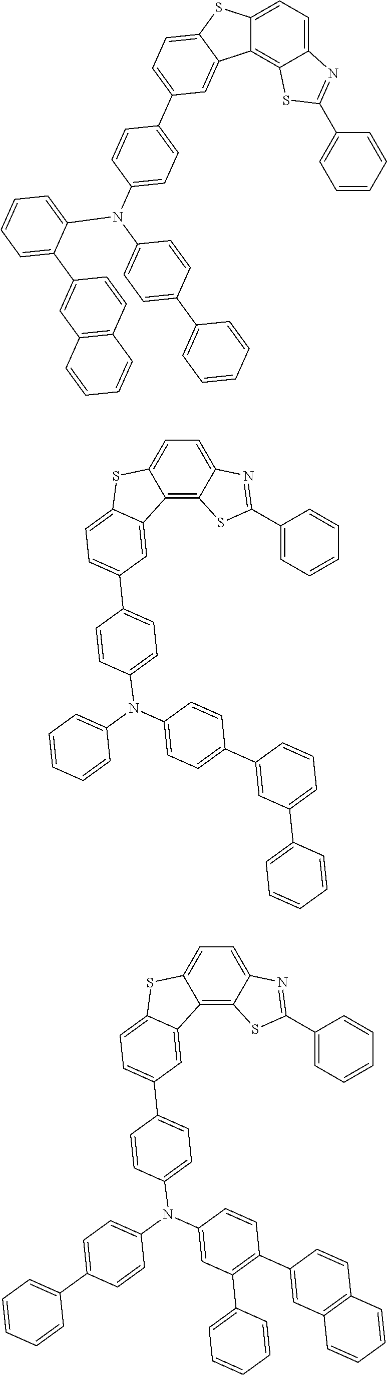 [SMILES: c1ccc(-c2ccc(N(c3ccc(-c4ccc5sc6ccc7nc(-c8ccccc8)sc7c6c5c4)cc3)c3ccccc3-c3ccc4ccccc4c3)cc2)cc1 ; c1ccc(-c2cccc(-c3ccc(N(c4ccccc4)c4ccc(-c5ccc6sc7ccc8nc(-c9ccccc9)sc8c7c6c5)cc4)cc3)c2)cc1 ; c1ccc(-c2ccc(N(c3ccc(-c4ccc5sc6ccc7nc(-c8ccccc8)sc7c6c5c4)cc3)c3ccc(-c4ccc5ccccc5c4)c(-c4ccccc4)c3)cc2)cc1]

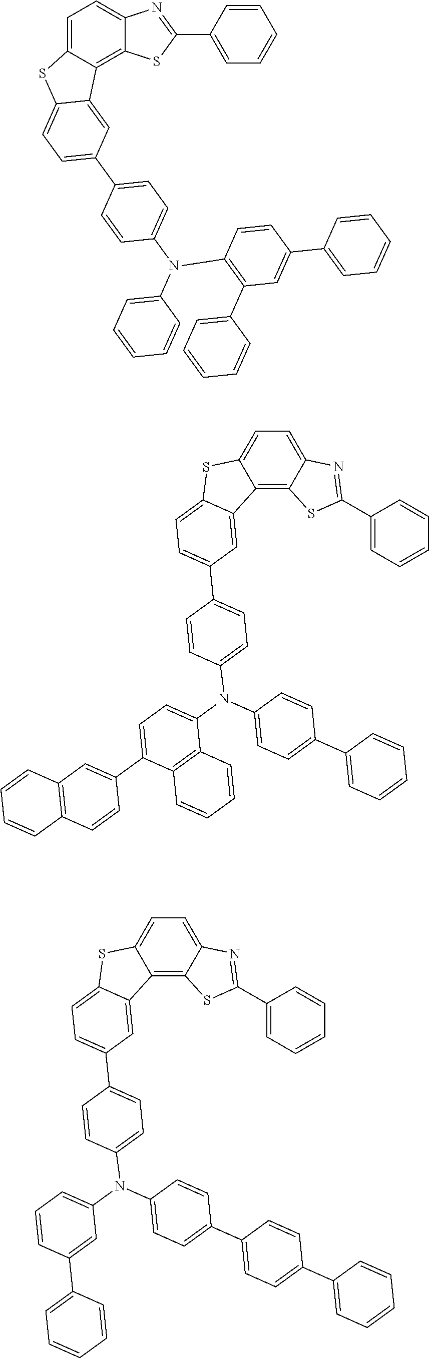 [SMILES: c1ccc(-c2ccc(N(c3ccccc3)c3ccc(-c4ccc5sc6ccc7nc(-c8ccccc8)sc7c6c5c4)cc3)c(-c3ccccc3)c2)cc1 ; c1ccc(-c2ccc(N(c3ccc(-c4ccc5sc6ccc7nc(-c8ccccc8)sc7c6c5c4)cc3)c3ccc(-c4ccc5ccccc5c4)c4ccccc34)cc2)cc1 ; c1ccc(-c2ccc(-c3ccc(N(c4ccc(-c5ccc6sc7ccc8nc(-c9ccccc9)sc8c7c6c5)cc4)c4cccc(-c5ccccc5)c4)cc3)cc2)cc1]

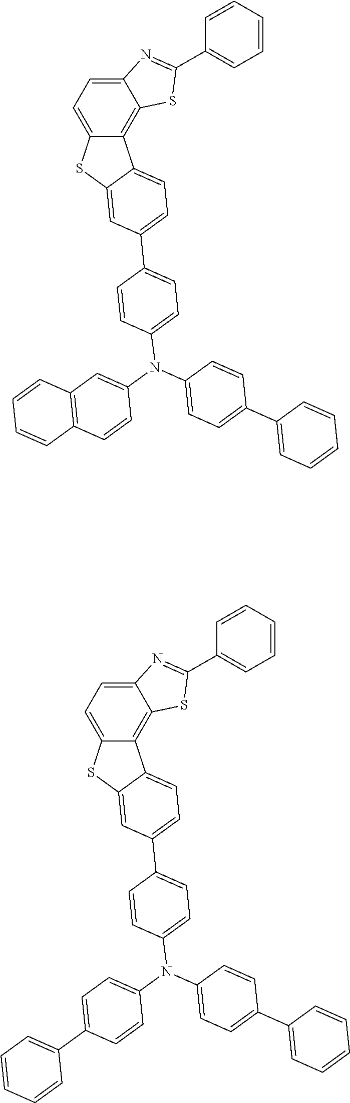 [SMILES: c1ccc(-c2ccc(N(c3ccc(-c4ccc5c(c4)sc4ccc6nc(-c7ccccc7)sc6c45)cc3)c3ccc4ccccc4c3)cc2)cc1 ; c1ccc(-c2ccc(N(c3ccc(-c4ccccc4)cc3)c3ccc(-c4ccc5c(c4)sc4ccc6nc(-c7ccccc7)sc6c45)cc3)cc2)cc1]

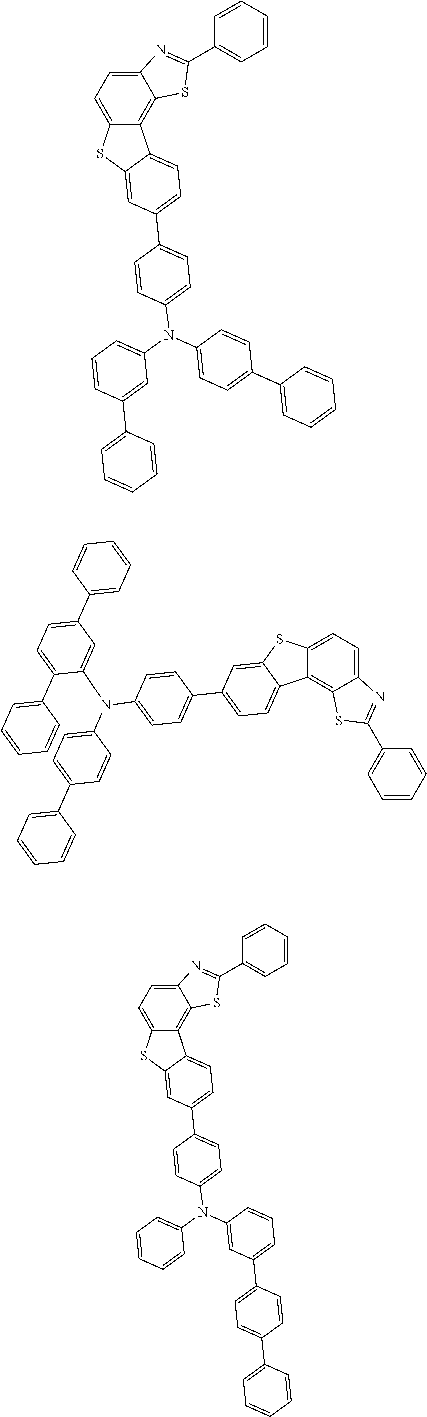 [SMILES: c1ccc(-c2ccc(N(c3ccc(-c4ccc5c(c4)sc4ccc6nc(-c7ccccc7)sc6c45)cc3)c3cccc(-c4ccccc4)c3)cc2)cc1 ; c1ccc(-c2ccc(N(c3ccc(-c4ccc5c(c4)sc4ccc6nc(-c7ccccc7)sc6c45)cc3)c3cc(-c4ccccc4)ccc3-c3ccccc3)cc2)cc1 ; c1ccc(-c2ccc(-c3cccc(N(c4ccccc4)c4ccc(-c5ccc6c(c5)sc5ccc7nc(-c8ccccc8)sc7c56)cc4)c3)cc2)cc1]

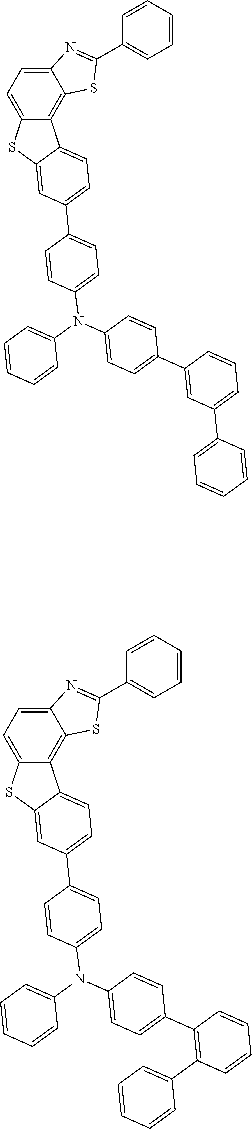 [SMILES: c1ccc(-c2cccc(-c3ccc(N(c4ccccc4)c4ccc(-c5ccc6c(c5)sc5ccc7nc(-c8ccccc8)sc7c56)cc4)cc3)c2)cc1 ; c1ccc(-c2nc3ccc4sc5cc(-c6ccc(N(c7ccccc7)c7ccc(-c8ccccc8-c8ccccc8)cc7)cc6)ccc5c4c3s2)cc1]

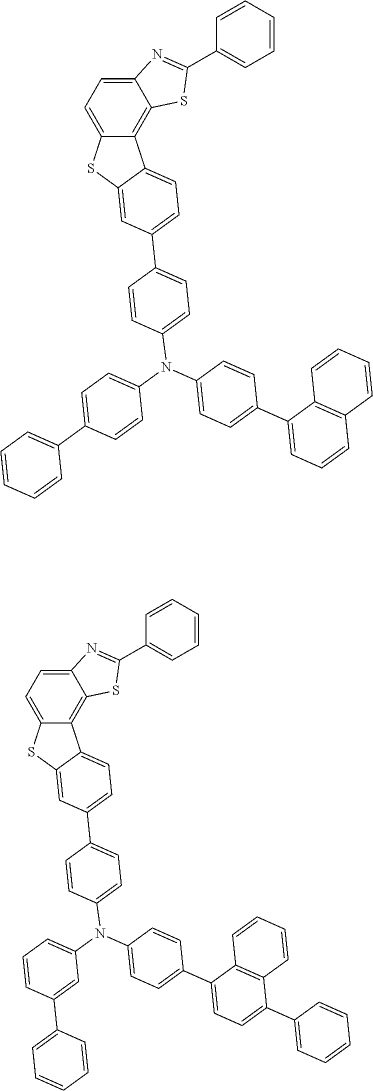 [SMILES: c1ccc(-c2ccc(N(c3ccc(-c4ccc5c(c4)sc4ccc6nc(-c7ccccc7)sc6c45)cc3)c3ccc(-c4cccc5ccccc45)cc3)cc2)cc1 ; c1ccc(-c2cccc(N(c3ccc(-c4ccc5c(c4)sc4ccc6nc(-c7ccccc7)sc6c45)cc3)c3ccc(-c4ccc(-c5ccccc5)c5ccccc45)cc3)c2)cc1]

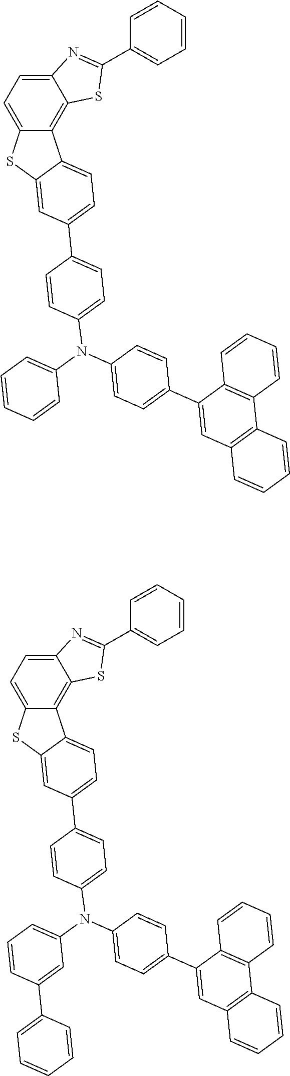 [SMILES: c1ccc(-c2nc3ccc4sc5cc(-c6ccc(N(c7ccccc7)c7ccc(-c8cc9ccccc9c9ccccc89)cc7)cc6)ccc5c4c3s2)cc1 ; c1ccc(-c2cccc(N(c3ccc(-c4ccc5c(c4)sc4ccc6nc(-c7ccccc7)sc6c45)cc3)c3ccc(-c4cc5ccccc5c5ccccc45)cc3)c2)cc1]

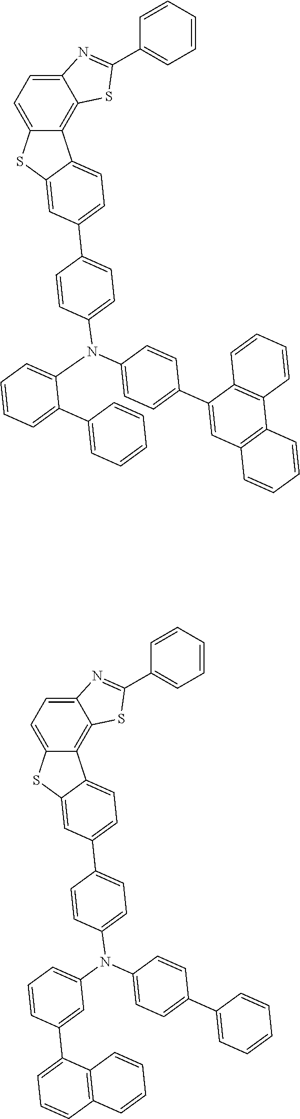 [SMILES: c1ccc(-c2nc3ccc4sc5cc(-c6ccc(N(c7ccc(-c8cc9ccccc9c9ccccc89)cc7)c7ccccc7-c7ccccc7)cc6)ccc5c4c3s2)cc1 ; c1ccc(-c2ccc(N(c3ccc(-c4ccc5c(c4)sc4ccc6nc(-c7ccccc7)sc6c45)cc3)c3cccc(-c4cccc5ccccc45)c3)cc2)cc1]

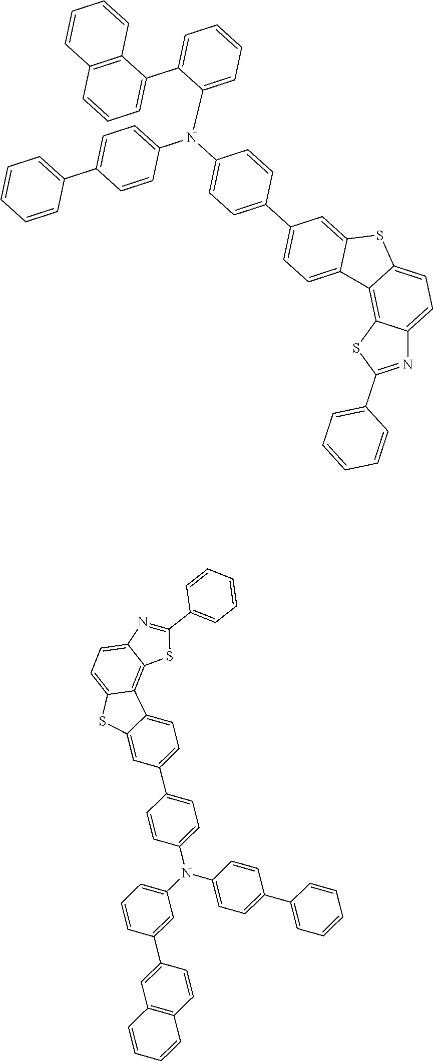 [SMILES: c1ccc(-c2ccc(N(c3ccc(-c4ccc5c(c4)sc4ccc6nc(-c7ccccc7)sc6c45)cc3)c3ccccc3-c3cccc4ccccc34)cc2)cc1 ; c1ccc(-c2ccc(N(c3ccc(-c4ccc5c(c4)sc4ccc6nc(-c7ccccc7)sc6c45)cc3)c3cccc(-c4ccc5ccccc5c4)c3)cc2)cc1]

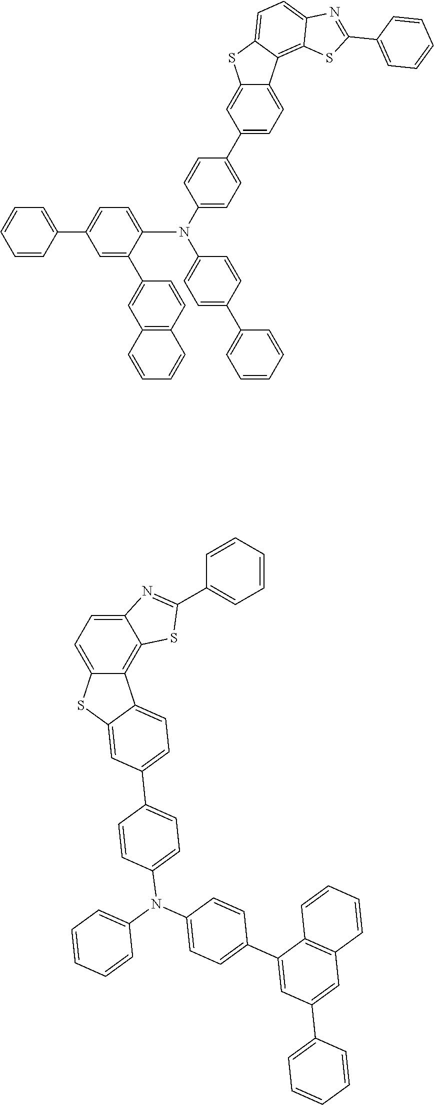 [SMILES: c1ccc(-c2ccc(N(c3ccc(-c4ccc5c(c4)sc4ccc6nc(-c7ccccc7)sc6c45)cc3)c3ccc(-c4ccccc4)cc3-c3ccc4ccccc4c3)cc2)cc1 ; c1ccc(-c2cc(-c3ccc(N(c4ccccc4)c4ccc(-c5ccc6c(c5)sc5ccc7nc(-c8ccccc8)sc7c56)cc4)cc3)c3ccccc3c2)cc1]

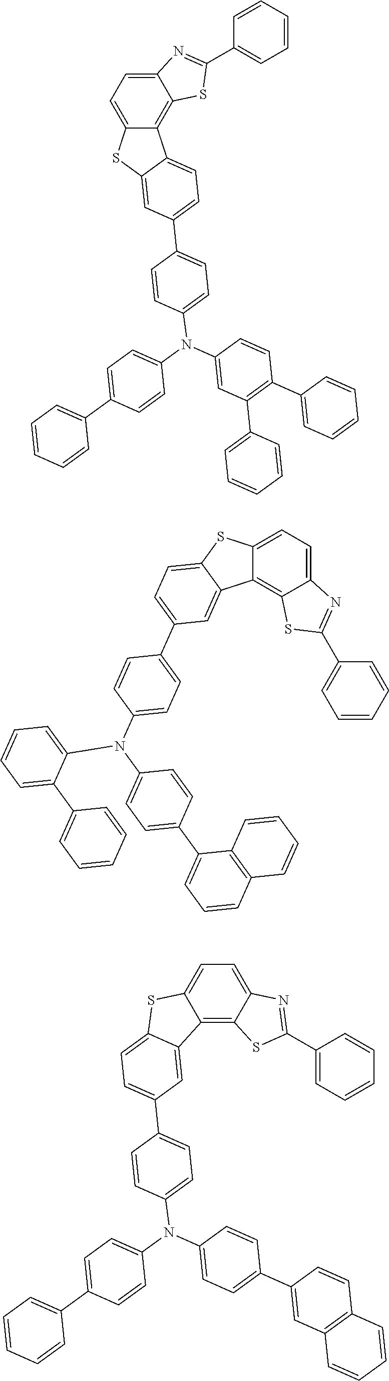

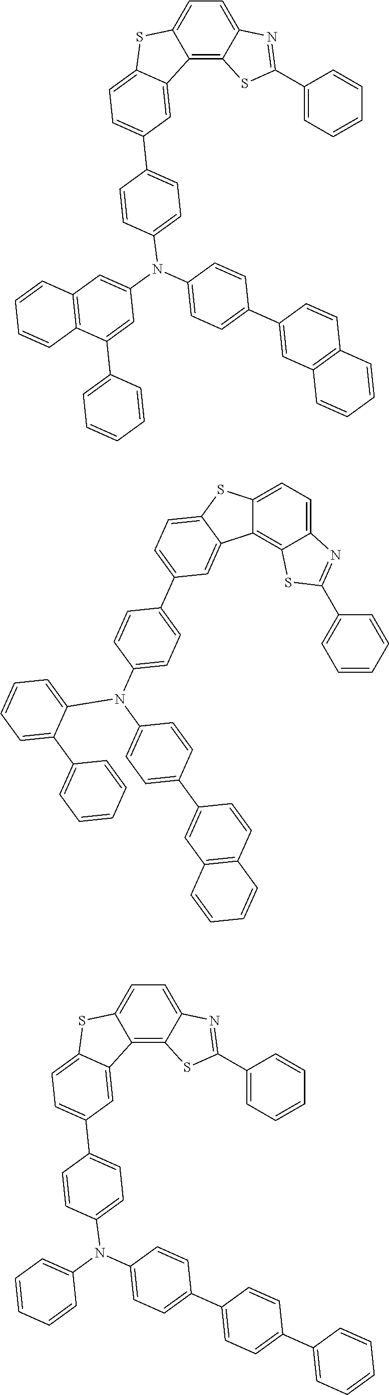 [SMILES: c1ccc(-c2nc3ccc4sc5ccc(-c6ccc(N(c7ccc(-c8ccc9ccccc9c8)cc7)c7cc(-c8ccccc8)c8ccccc8c7)cc6)cc5c4c3s2)cc1 ; c1ccc(-c2nc3ccc4sc5ccc(-c6ccc(N(c7ccc(-c8ccc9ccccc9c8)cc7)c7ccccc7-c7ccccc7)cc6)cc5c4c3s2)cc1 ; c1ccc(-c2ccc(-c3ccc(N(c4ccccc4)c4ccc(-c5ccc6sc7ccc8nc(-c9ccccc9)sc8c7c6c5)cc4)cc3)cc2)cc1]

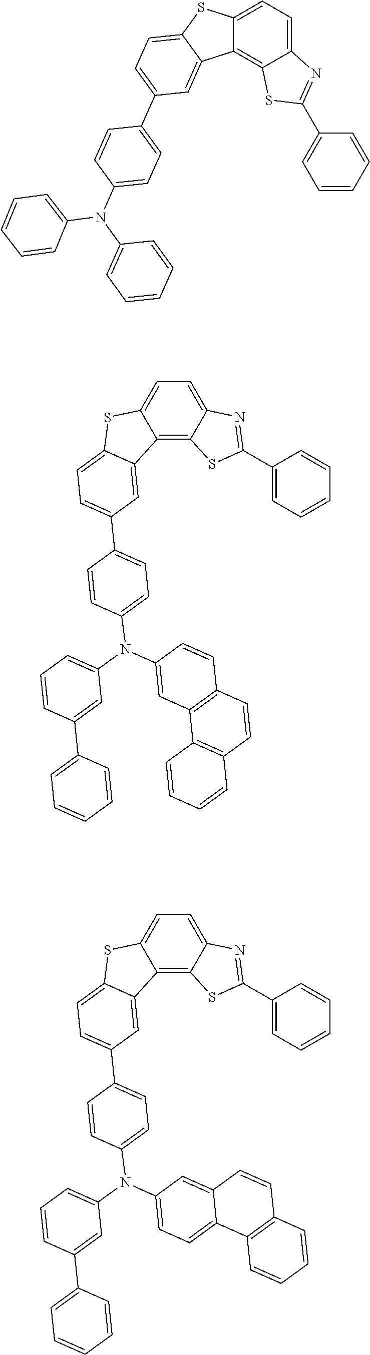 [SMILES: c1ccc(-c2nc3ccc4sc5ccc(-c6ccc(N(c7ccccc7)c7ccccc7)cc6)cc5c4c3s2)cc1 ; c1ccc(-c2cccc(N(c3ccc(-c4ccc5sc6ccc7nc(-c8ccccc8)sc7c6c5c4)cc3)c3ccc4ccc5ccccc5c4c3)c2)cc1 ; c1ccc(-c2cccc(N(c3ccc(-c4ccc5sc6ccc7nc(-c8ccccc8)sc7c6c5c4)cc3)c3ccc4c(ccc5ccccc54)c3)c2)cc1]

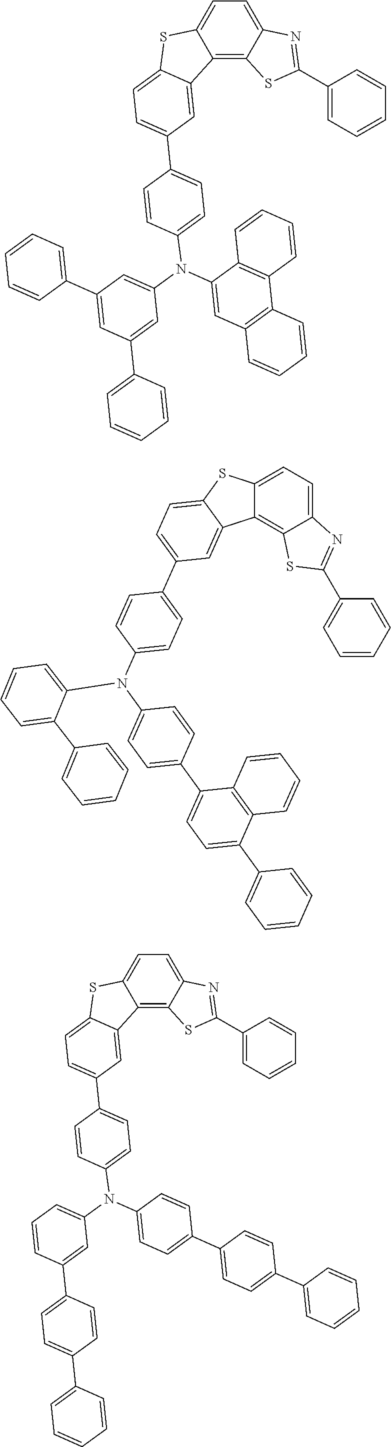 [SMILES: c1ccc(-c2cc(-c3ccccc3)cc(N(c3ccc(-c4ccc5sc6ccc7nc(-c8ccccc8)sc7c6c5c4)cc3)c3cc4ccccc4c4ccccc34)c2)cc1 ; c1ccc(-c2nc3ccc4sc5ccc(-c6ccc(N(c7ccc(-c8ccc(-c9ccccc9)c9ccccc89)cc7)c7ccccc7-c7ccccc7)cc6)cc5c4c3s2)cc1 ; c1ccc(-c2ccc(-c3ccc(N(c4ccc(-c5ccc6sc7ccc8nc(-c9ccccc9)sc8c7c6c5)cc4)c4cccc(-c5ccc(-c6ccccc6)cc5)c4)cc3)cc2)cc1]

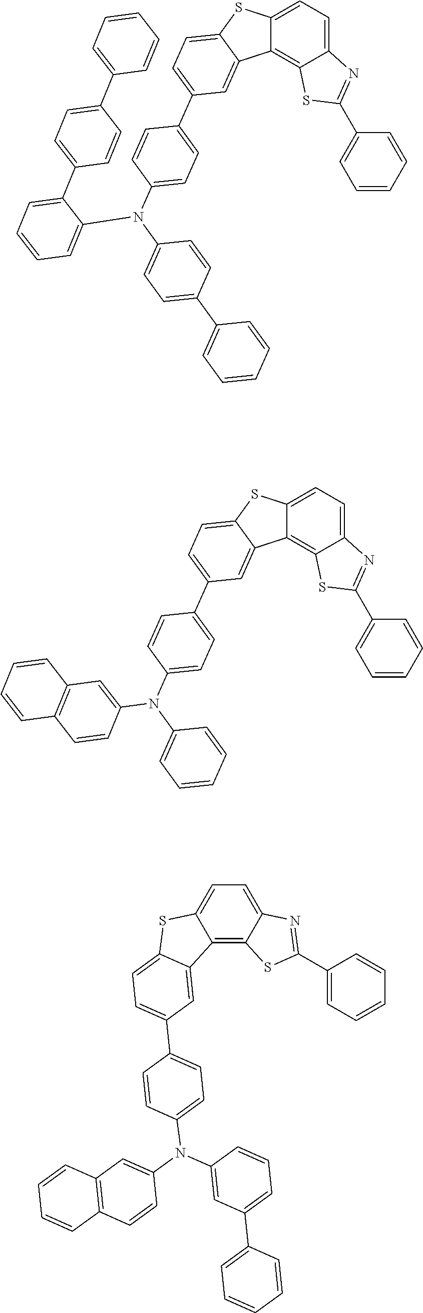 [SMILES: c1ccc(-c2ccc(-c3ccccc3N(c3ccc(-c4ccccc4)cc3)c3ccc(-c4ccc5sc6ccc7nc(-c8ccccc8)sc7c6c5c4)cc3)cc2)cc1 ; c1ccc(-c2nc3ccc4sc5ccc(-c6ccc(N(c7ccccc7)c7ccc8ccccc8c7)cc6)cc5c4c3s2)cc1 ; c1ccc(-c2cccc(N(c3ccc(-c4ccc5sc6ccc7nc(-c8ccccc8)sc7c6c5c4)cc3)c3ccc4ccccc4c3)c2)cc1]

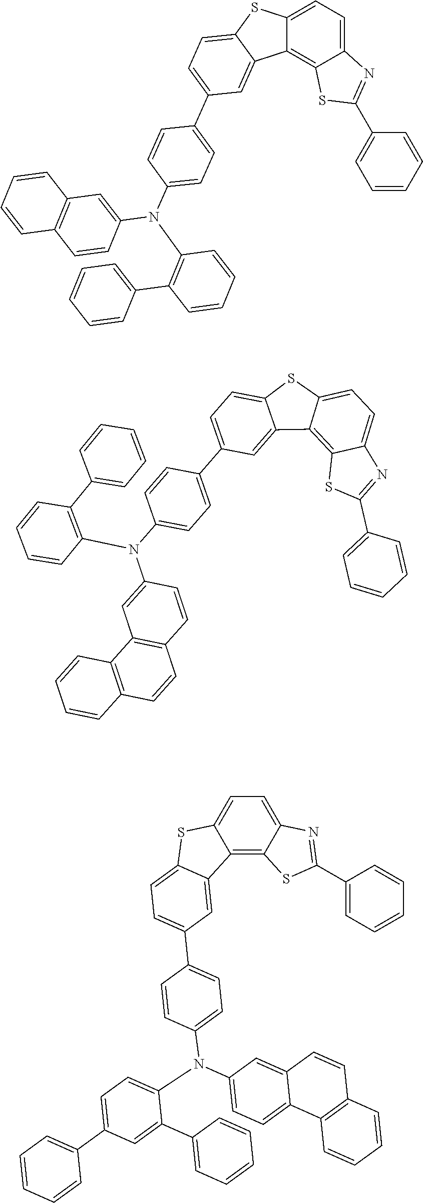 [SMILES: c1ccc(-c2nc3ccc4sc5ccc(-c6ccc(N(c7ccc8ccccc8c7)c7ccccc7-c7ccccc7)cc6)cc5c4c3s2)cc1 ; c1ccc(-c2nc3ccc4sc5ccc(-c6ccc(N(c7ccc8ccc9ccccc9c8c7)c7ccccc7-c7ccccc7)cc6)cc5c4c3s2)cc1 ; c1ccc(-c2ccc(N(c3ccc(-c4ccc5sc6ccc7nc(-c8ccccc8)sc7c6c5c4)cc3)c3ccc4c(ccc5ccccc54)c3)c(-c3ccccc3)c2)cc1]

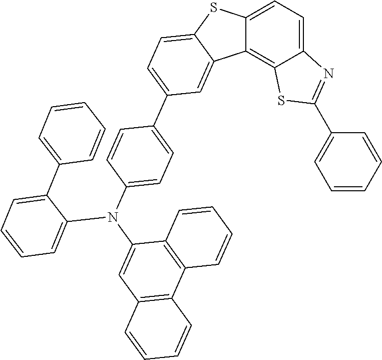 [SMILES: c1ccc(-c2nc3ccc4sc5ccc(-c6ccc(N(c7ccccc7-c7ccccc7)c7cc8ccccc8c8ccccc78)cc6)cc5c4c3s2)cc1]

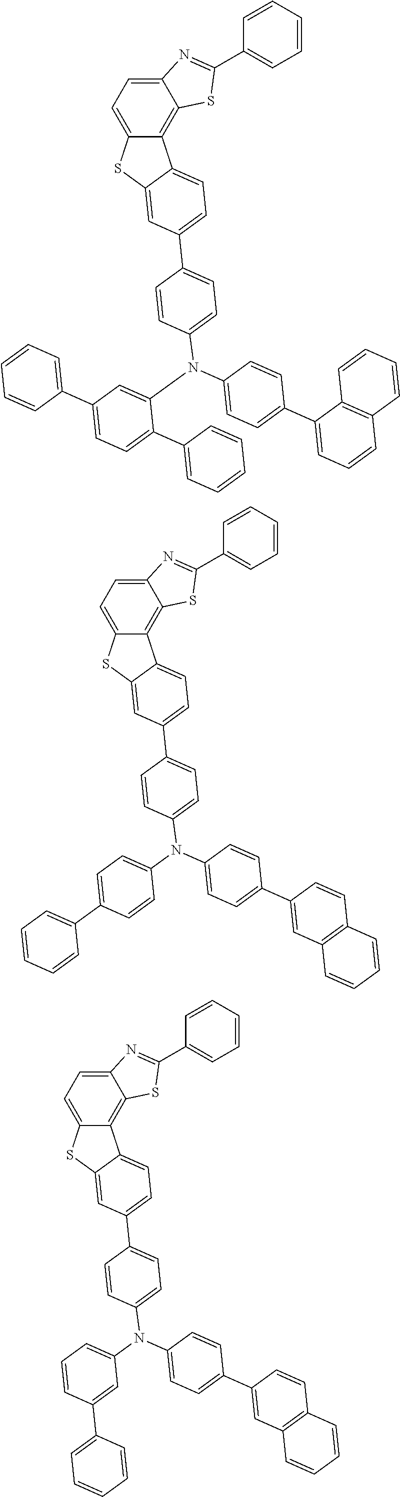

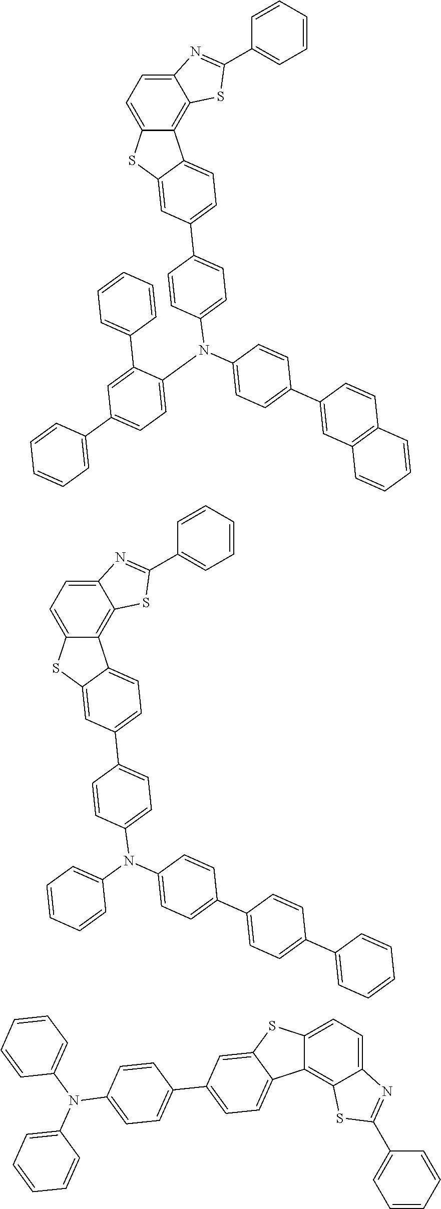

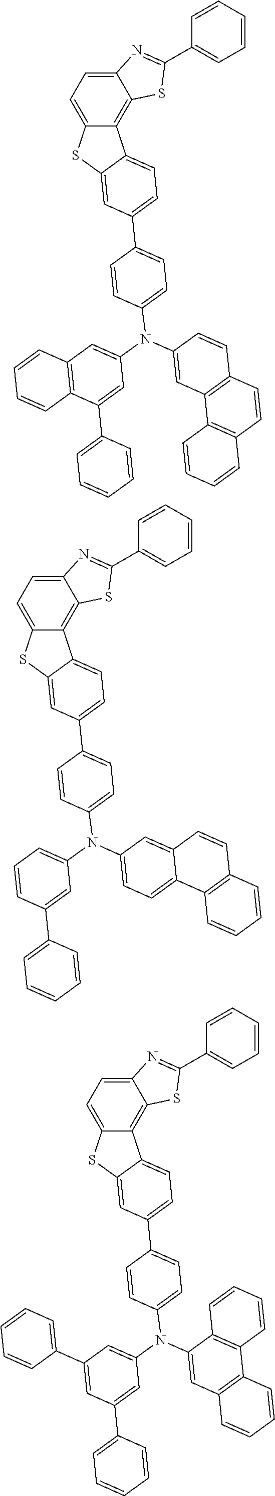

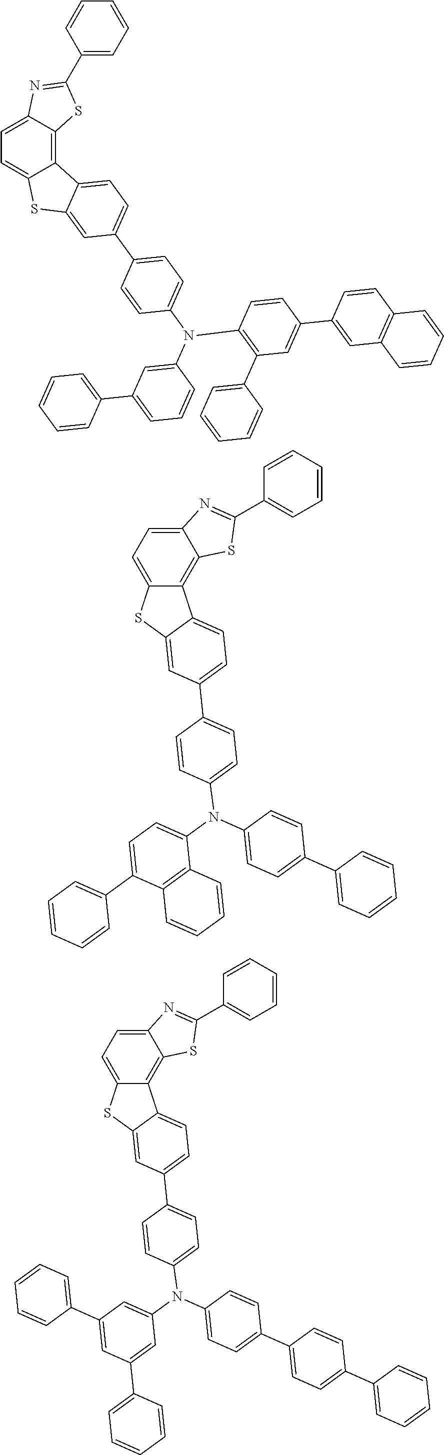

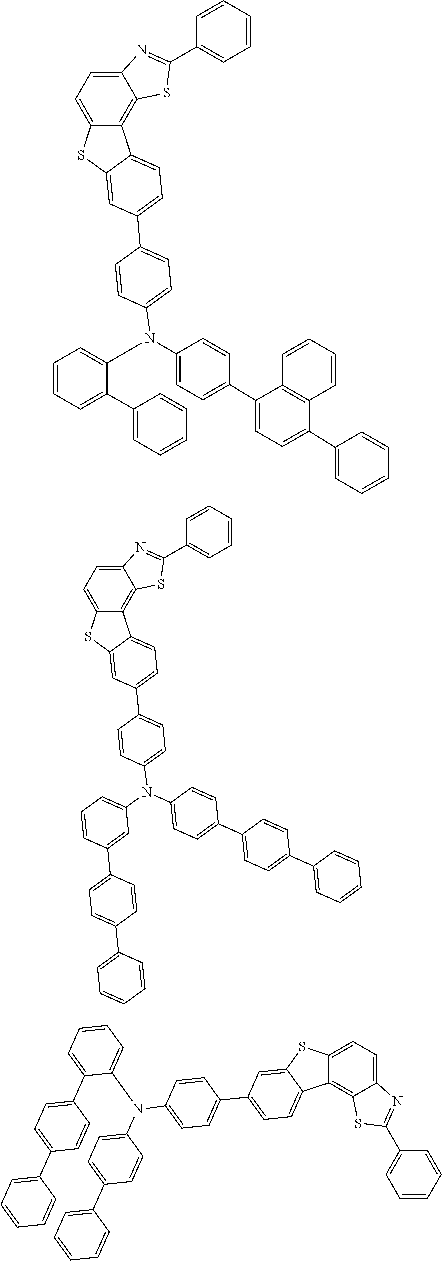

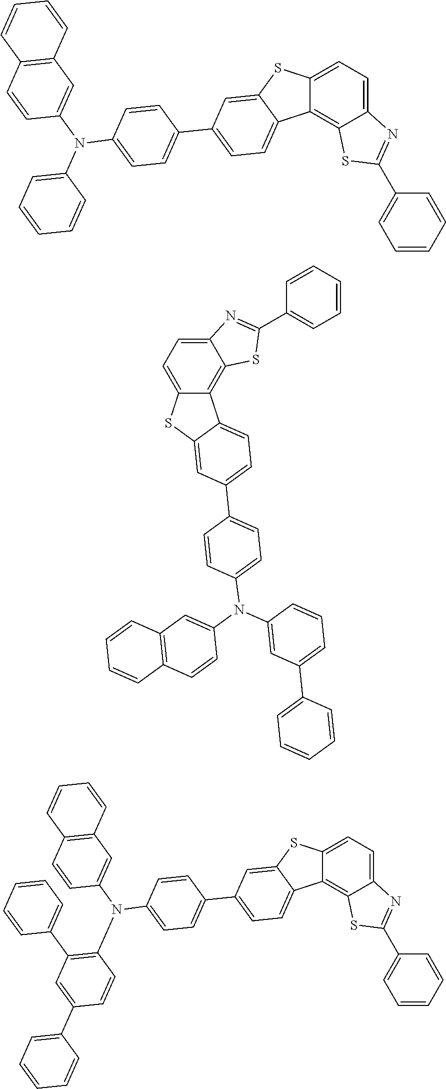 [SMILES: c1ccc(-c2nc3ccc4sc5cc(-c6ccc(N(c7ccccc7)c7ccc8ccccc8c7)cc6)ccc5c4c3s2)cc1 ; c1ccc(-c2cccc(N(c3ccc(-c4ccc5c(c4)sc4ccc6nc(-c7ccccc7)sc6c45)cc3)c3ccc4ccccc4c3)c2)cc1 ; c1ccc(-c2ccc(N(c3ccc(-c4ccc5c(c4)sc4ccc6nc(-c7ccccc7)sc6c45)cc3)c3ccc4ccccc4c3)c(-c3ccccc3)c2)cc1]

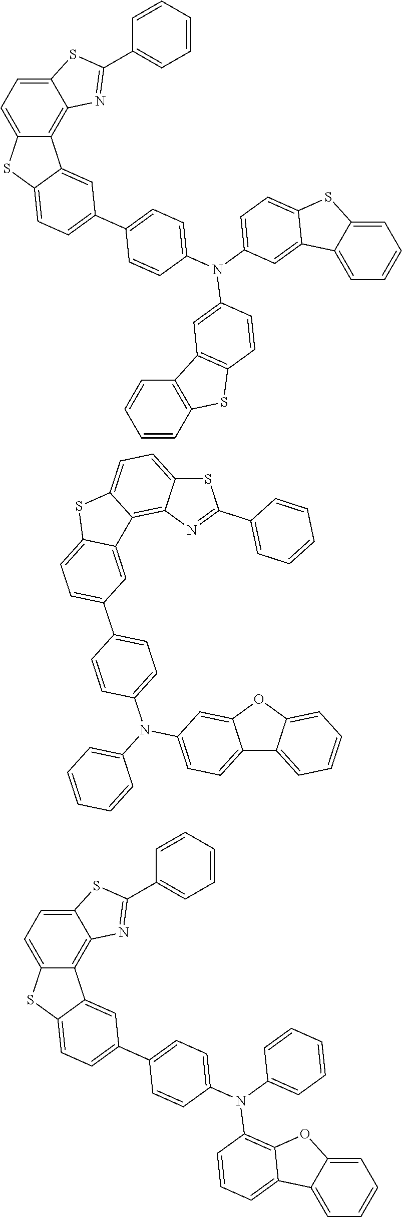

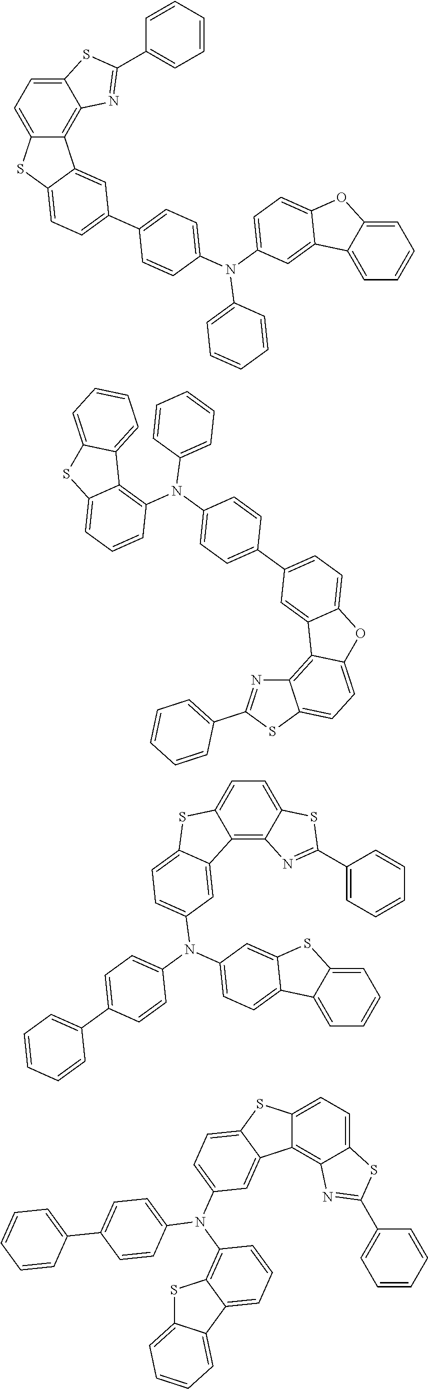

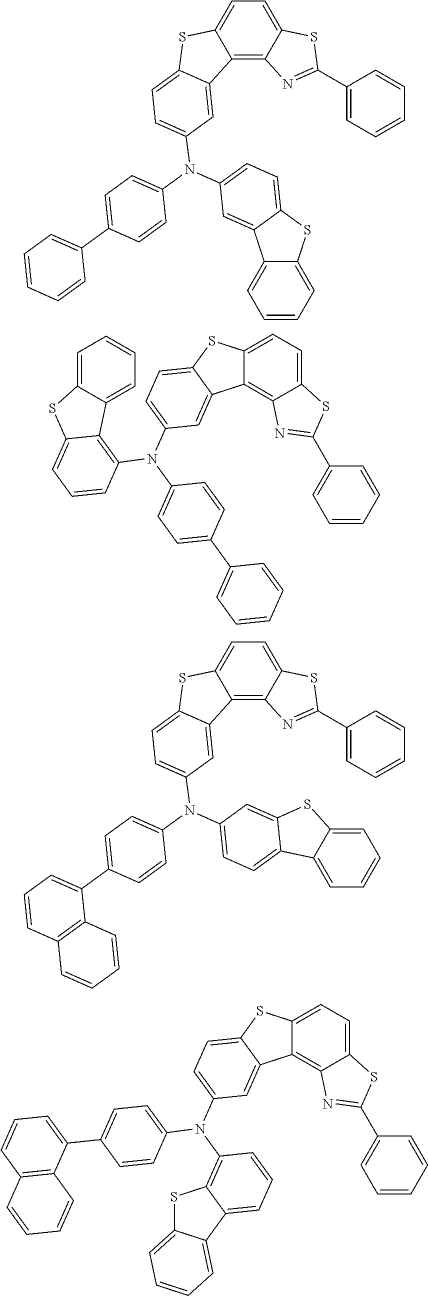

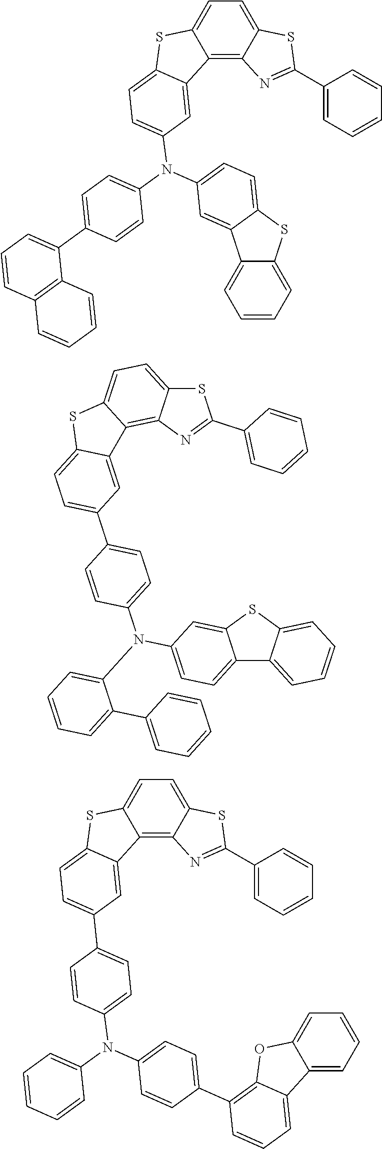

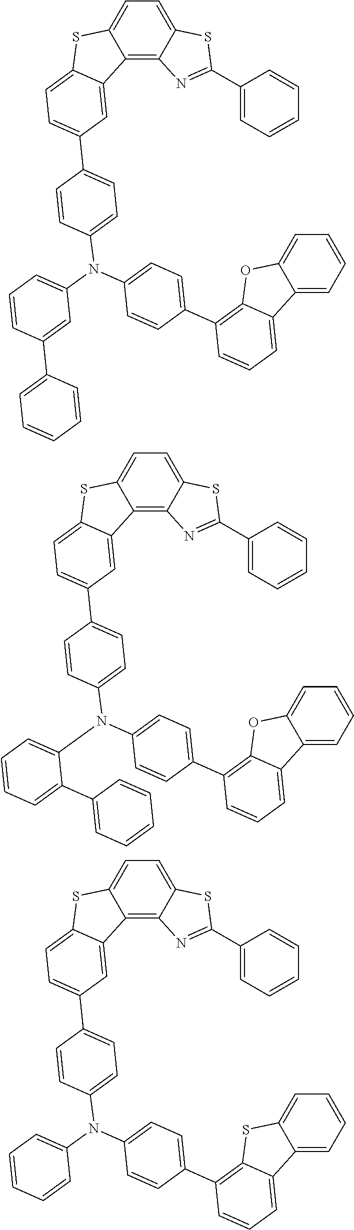

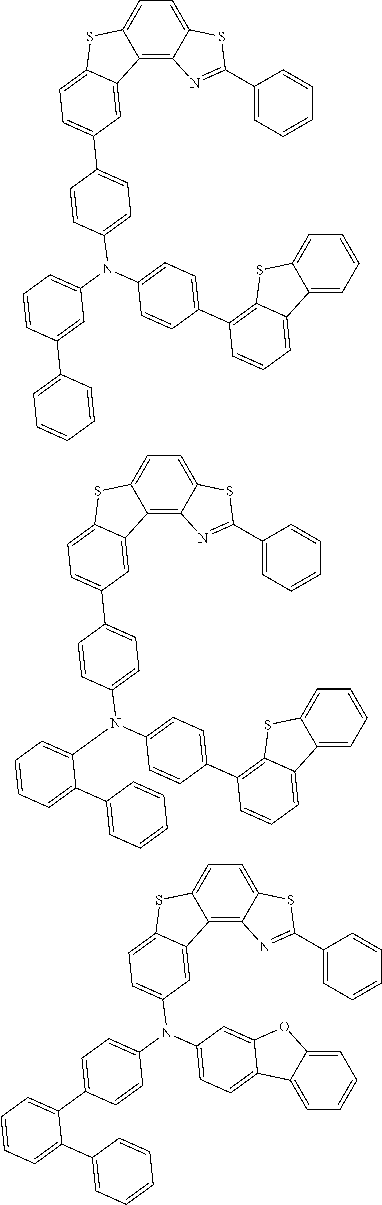 [SMILES: c1ccc(-c2cccc(N(c3ccc(-c4ccc5sc6ccc7sc(-c8ccccc8)nc7c6c5c4)cc3)c3ccc(-c4cccc5c4sc4ccccc45)cc3)c2)cc1 ; c1ccc(-c2nc3c(ccc4sc5ccc(-c6ccc(N(c7ccc(-c8cccc9c8sc8ccccc89)cc7)c7ccccc7-c7ccccc7)cc6)cc5c43)s2)cc1 ; c1ccc(-c2nc3c(ccc4sc5ccc(N(c6ccc(-c7ccccc7-c7ccccc7)cc6)c6ccc7c(c6)oc6ccccc67)cc5c43)s2)cc1]

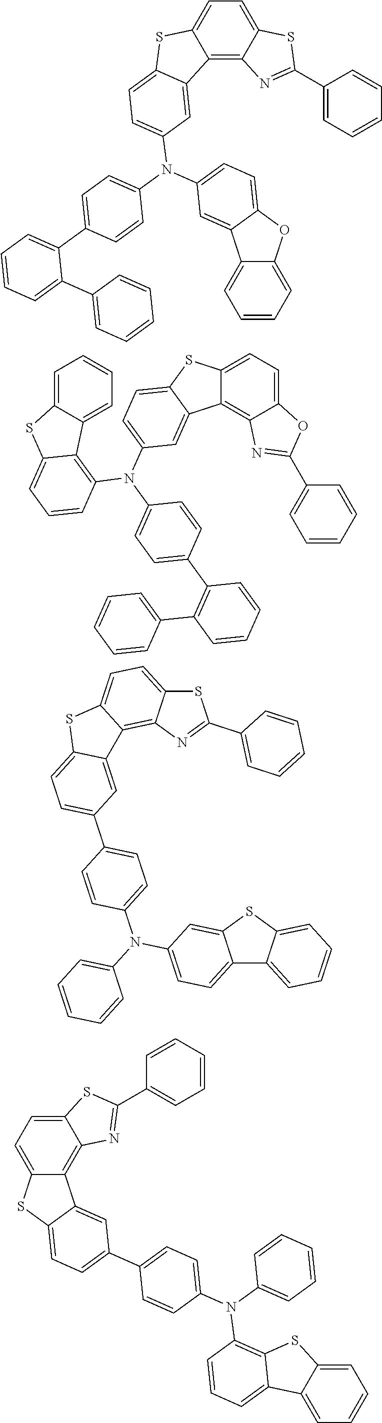

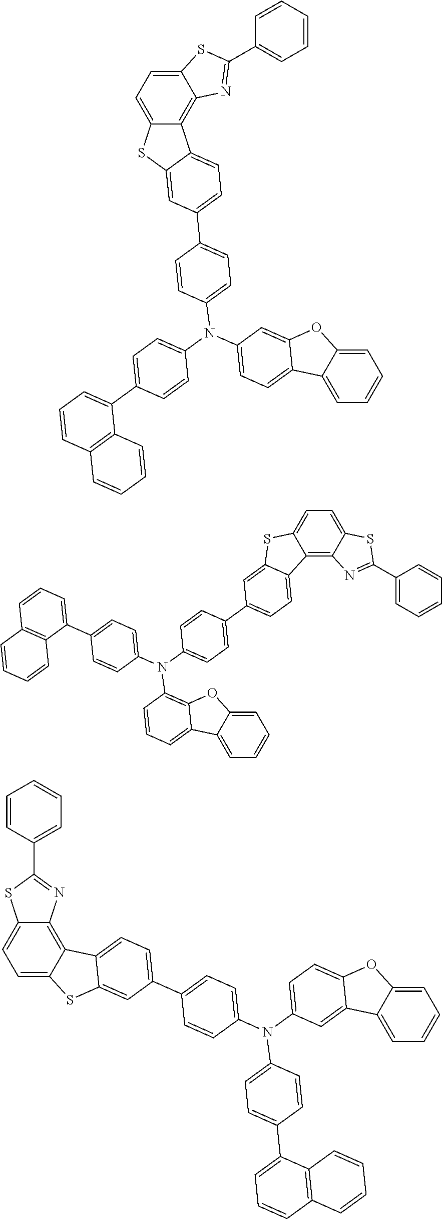 [SMILES: c1ccc(-c2nc3c(ccc4sc5cc(-c6ccc(N(c7ccc(-c8cccc9ccccc89)cc7)c7ccc8c(c7)oc7ccccc78)cc6)ccc5c43)s2)cc1 ; c1ccc(-c2nc3c(ccc4sc5cc(-c6ccc(N(c7ccc(-c8cccc9ccccc89)cc7)c7cccc8c7oc7ccccc78)cc6)ccc5c43)s2)cc1 ; c1ccc(-c2nc3c(ccc4sc5cc(-c6ccc(N(c7ccc(-c8cccc9ccccc89)cc7)c7ccc8oc9ccccc9c8c7)cc6)ccc5c43)s2)cc1]

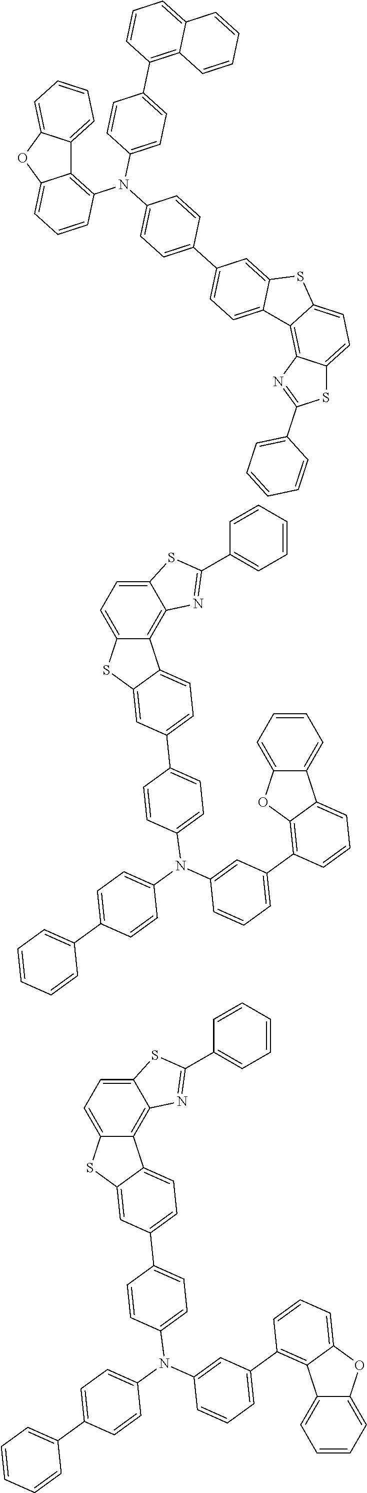

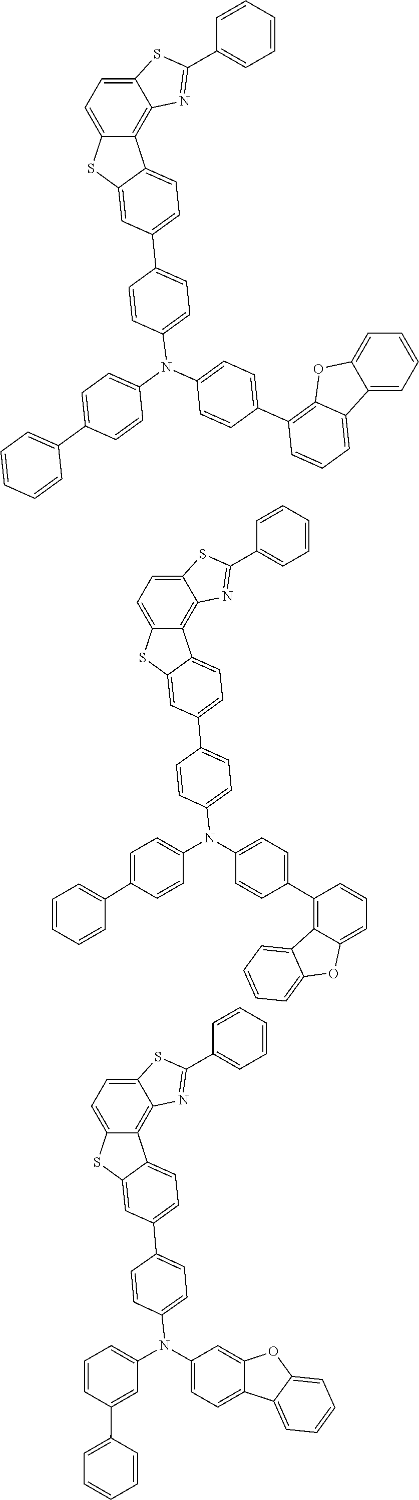

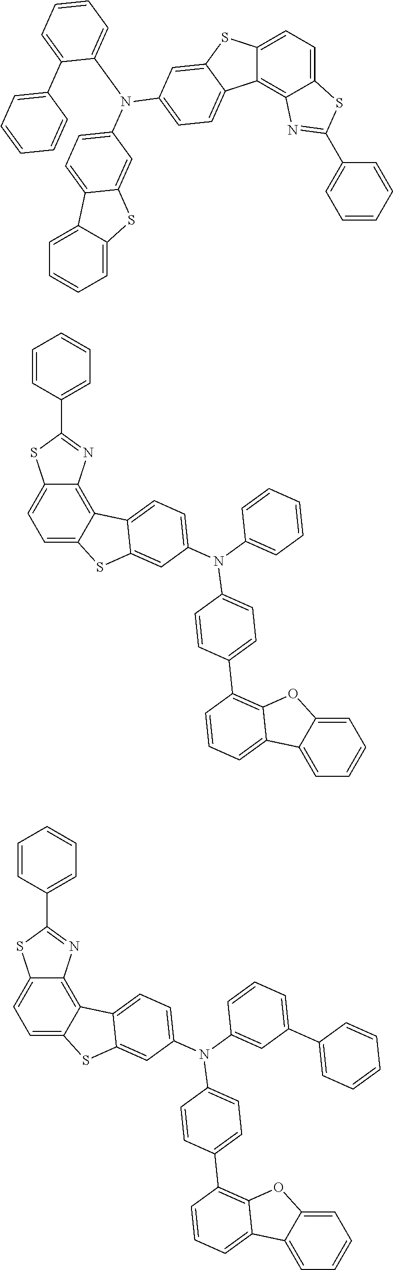

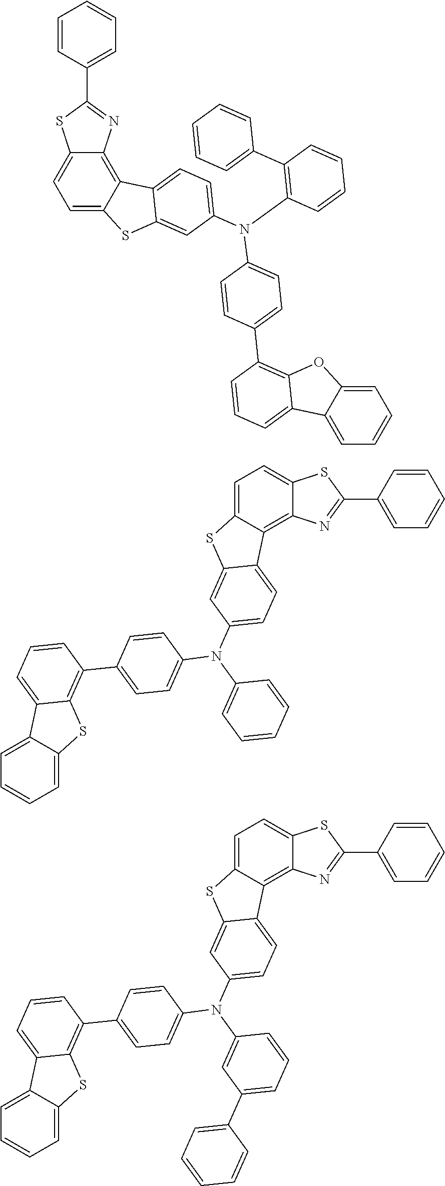

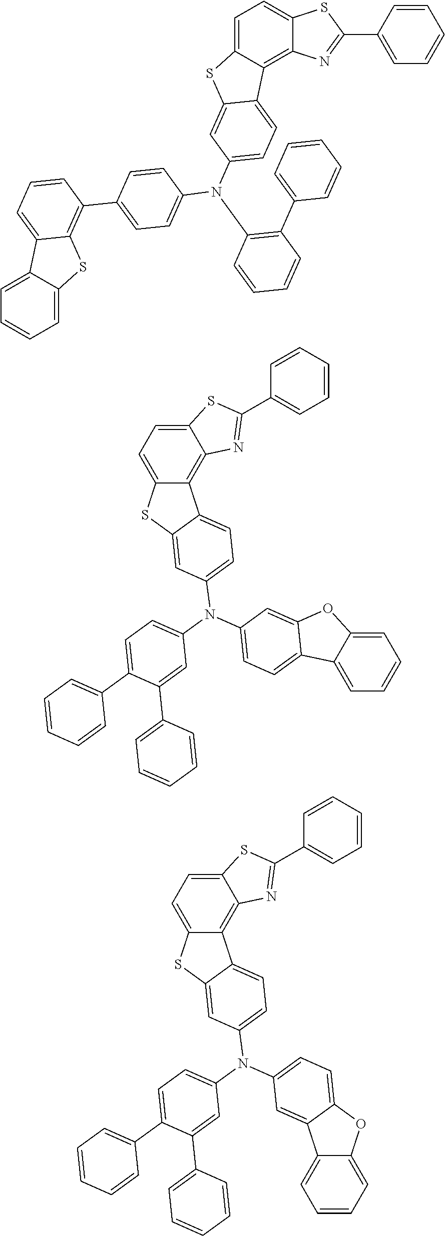 [SMILES: c1ccc(-c2nc3c(ccc4sc5cc(N(c6ccc(-c7cccc8c7sc7ccccc78)cc6)c6ccccc6-c6ccccc6)ccc5c43)s2)cc1 ; c1ccc(-c2nc3c(ccc4sc5cc(N(c6ccc(-c7ccccc7)c(-c7ccccc7)c6)c6ccc7c(c6)oc6ccccc67)ccc5c43)s2)cc1 ; c1ccc(-c2nc3c(ccc4sc5cc(N(c6ccc(-c7ccccc7)c(-c7ccccc7)c6)c6ccc7oc8ccccc8c7c6)ccc5c43)s2)cc1]

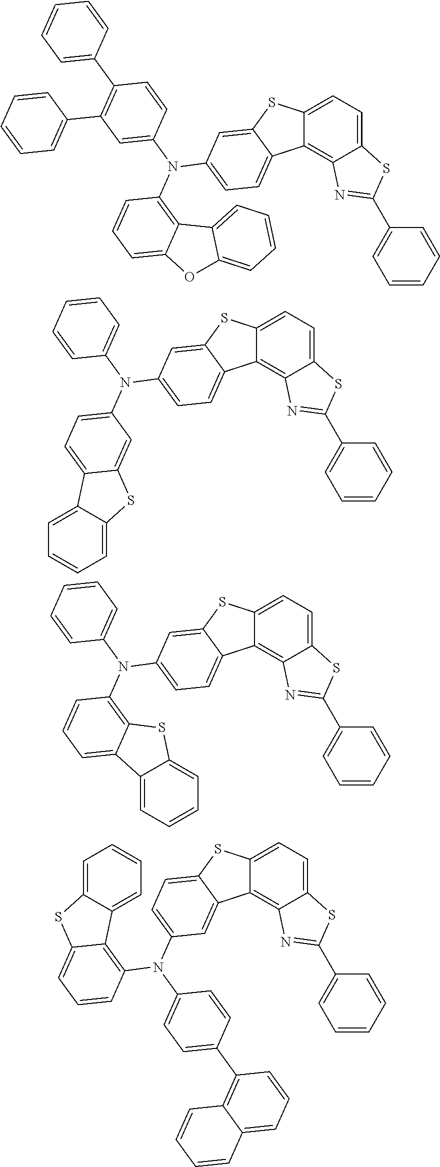

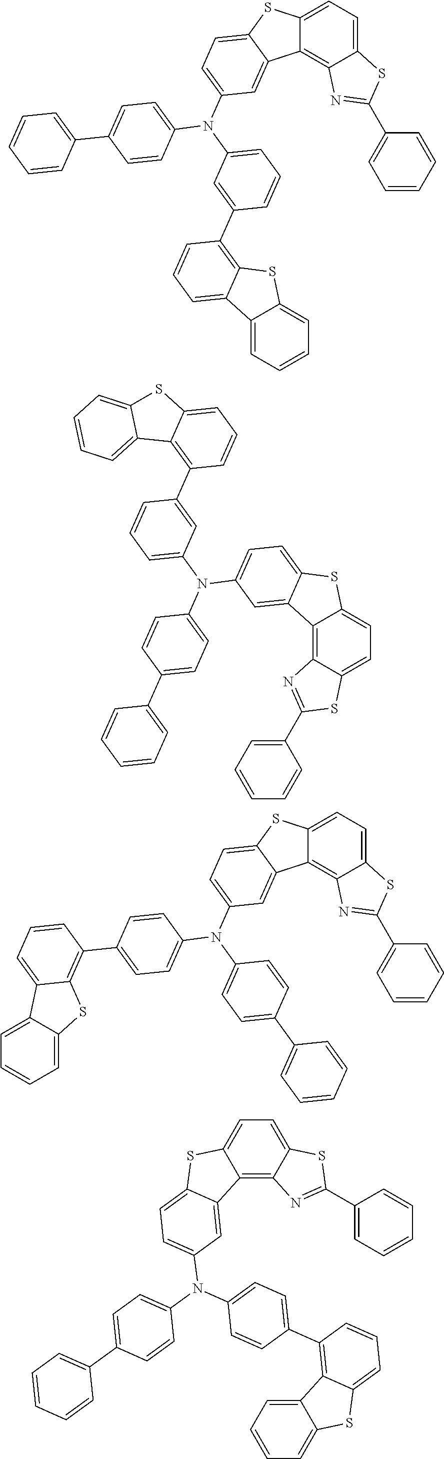

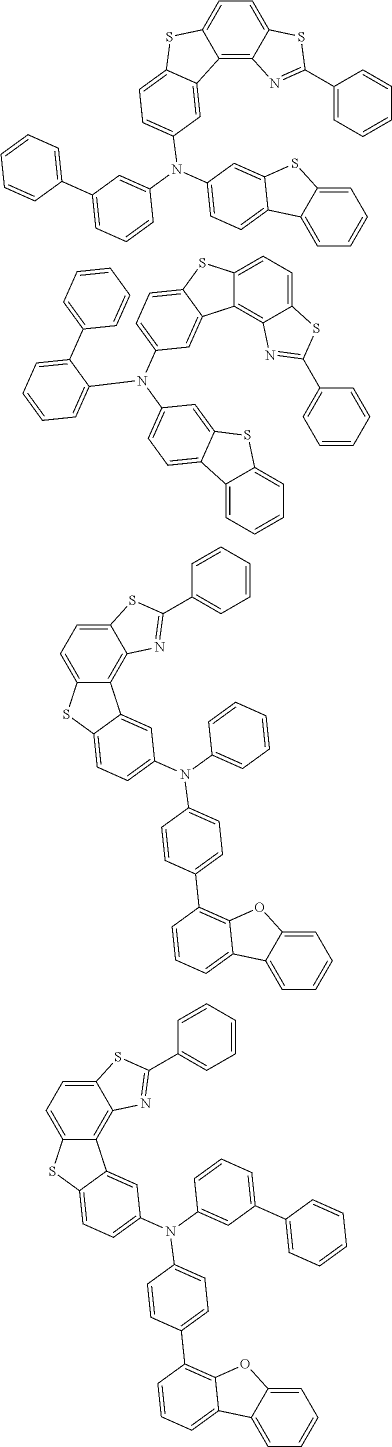

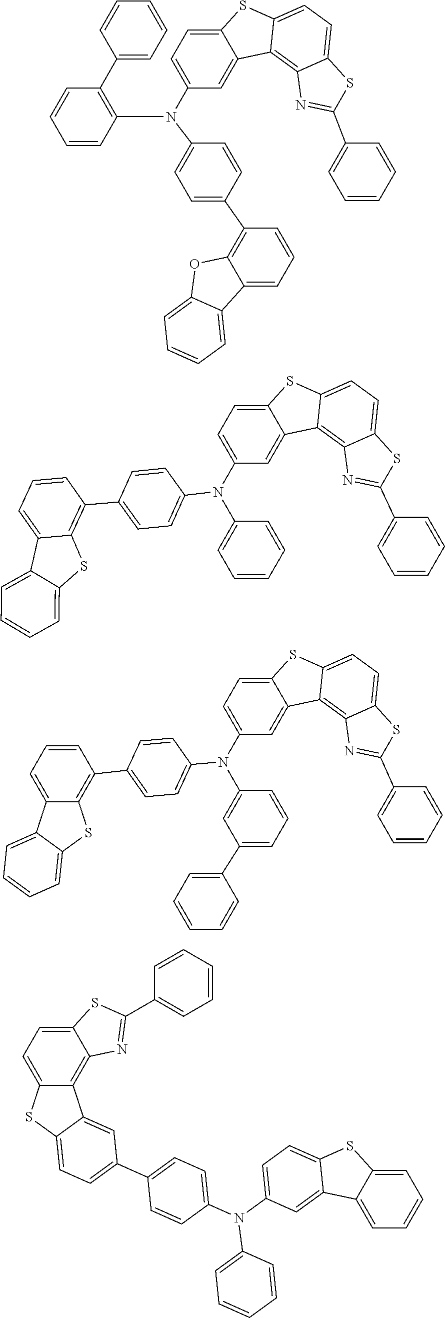

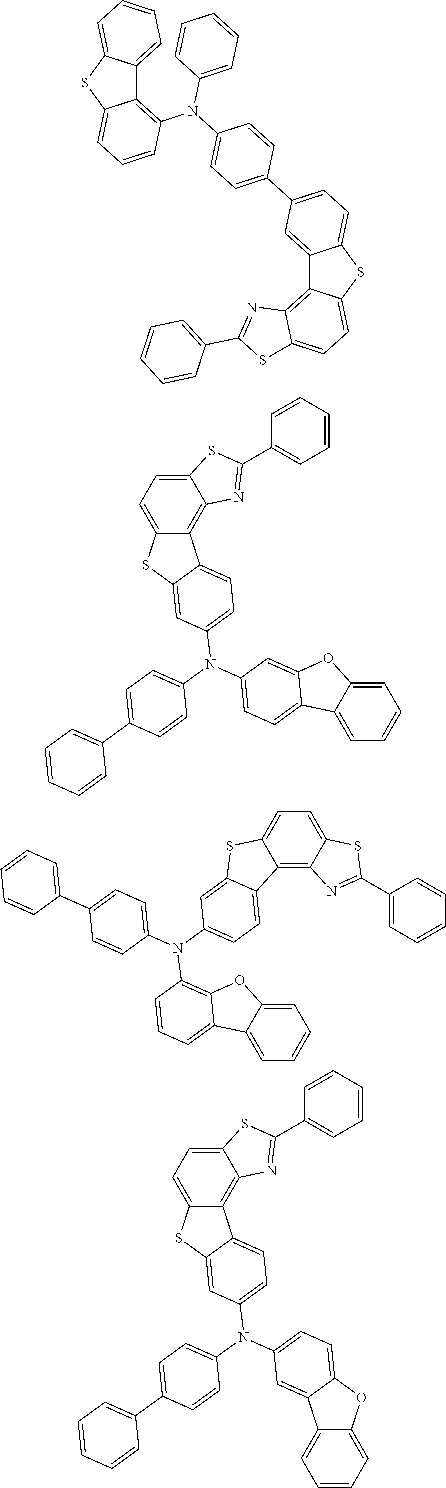

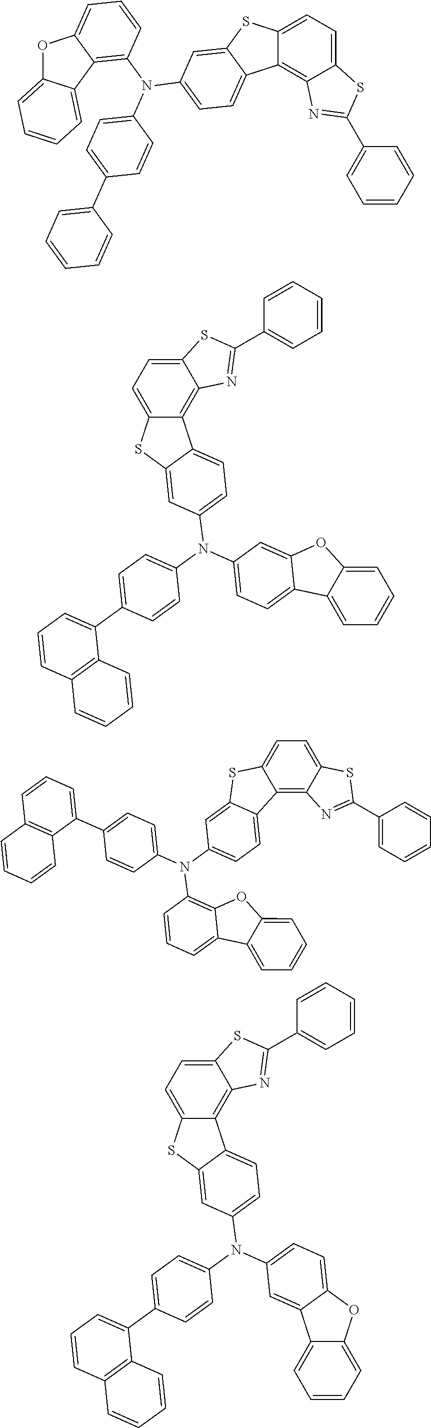

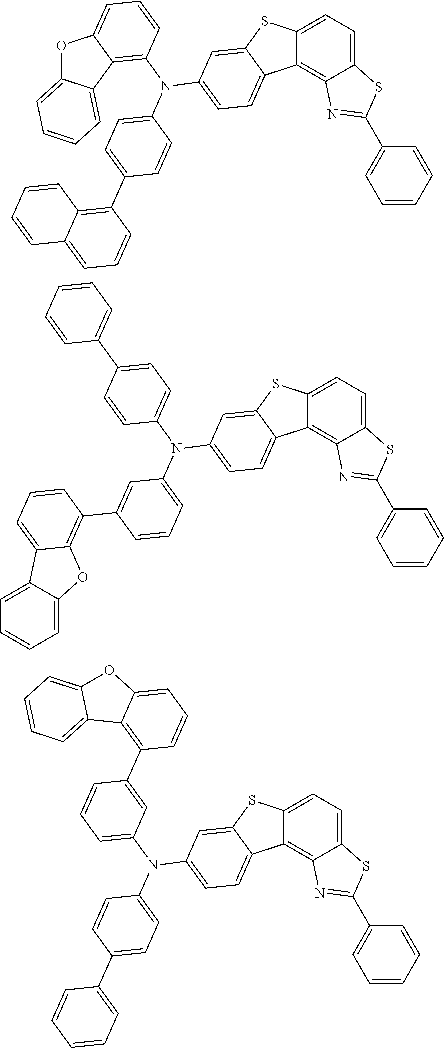

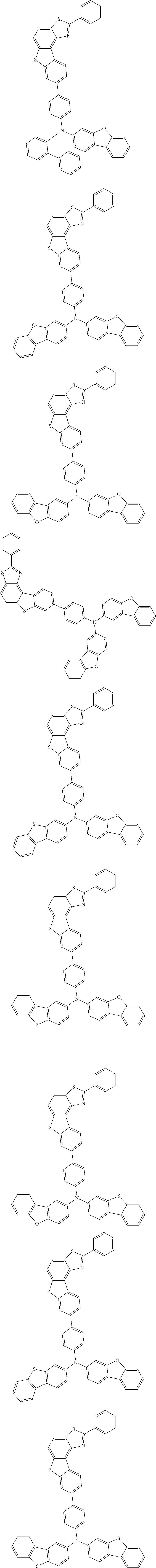

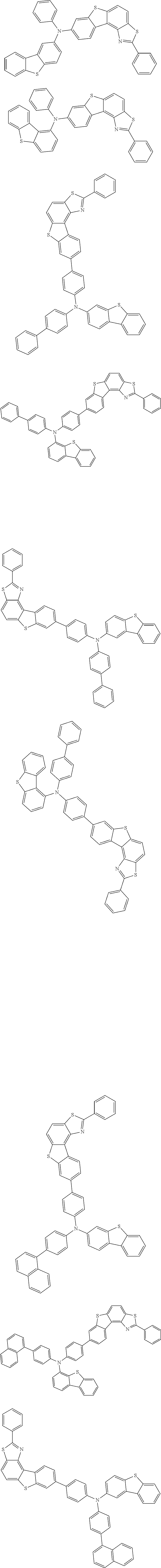

[chemical structure]

[chemical structure]

[chemical structure]

[chemical structure]

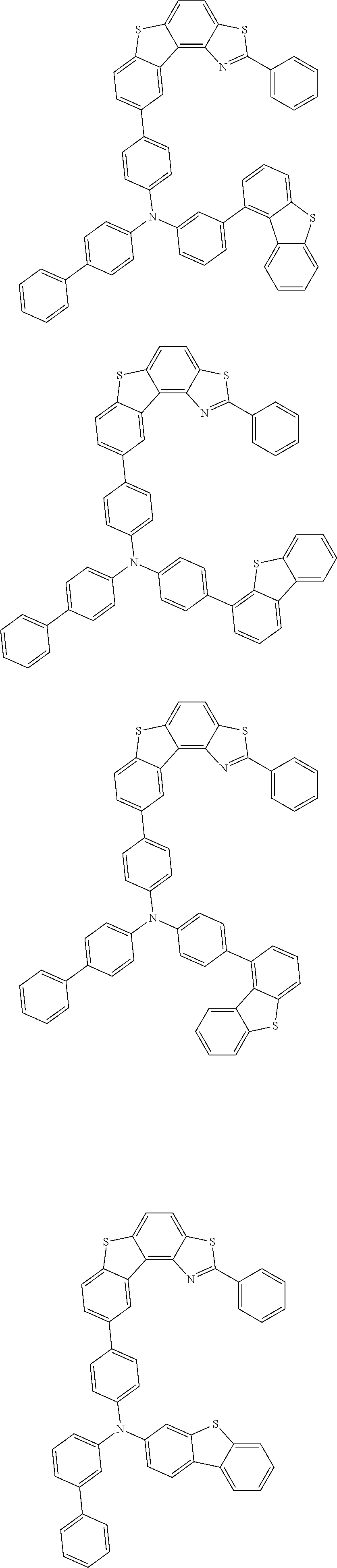 [SMILES: c1ccc(-c2ccc(N(c3ccc(-c4ccc5sc6ccc7sc(-c8ccccc8)nc7c6c5c4)cc3)c3cccc(-c4cccc5sc6ccccc6c45)c3)cc2)cc1 ; c1ccc(-c2ccc(N(c3ccc(-c4ccc5sc6ccc7sc(-c8ccccc8)nc7c6c5c4)cc3)c3ccc(-c4cccc5c4sc4ccccc45)cc3)cc2)cc1 ; c1ccc(-c2ccc(N(c3ccc(-c4ccc5sc6ccc7sc(-c8ccccc8)nc7c6c5c4)cc3)c3ccc(-c4cccc5sc6ccccc6c45)cc3)cc2)cc1 ; c1ccc(-c2cccc(N(c3ccc(-c4ccc5sc6ccc7sc(-c8ccccc8)nc7c6c5c4)cc3)c3ccc4c(c3)sc3ccccc34)c2)cc1]

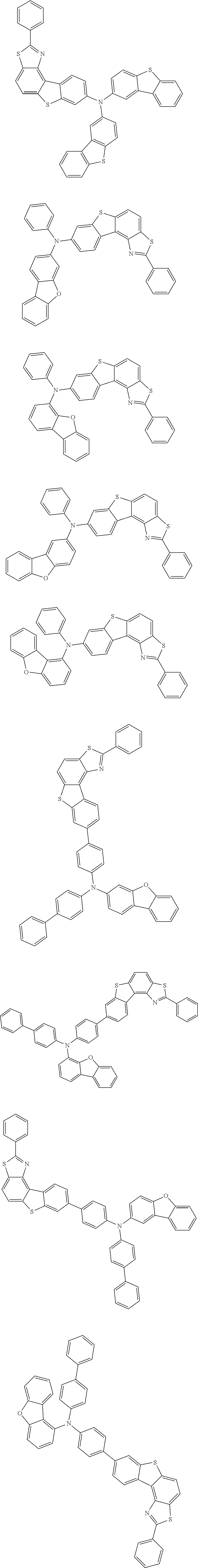

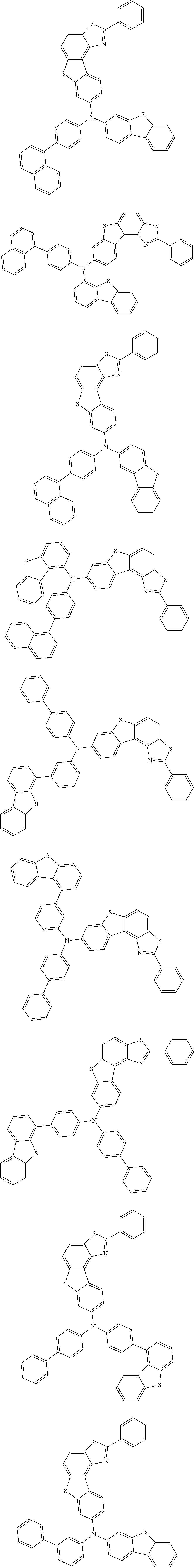

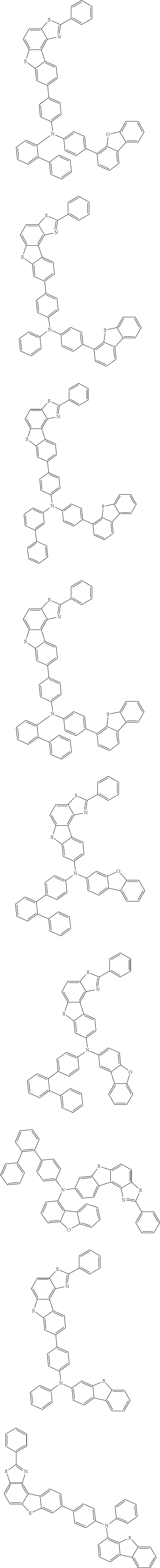

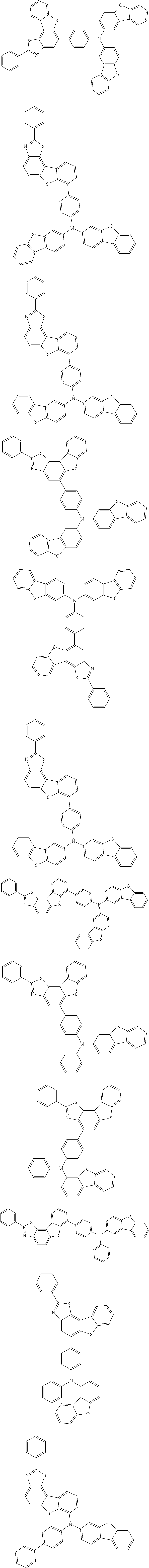

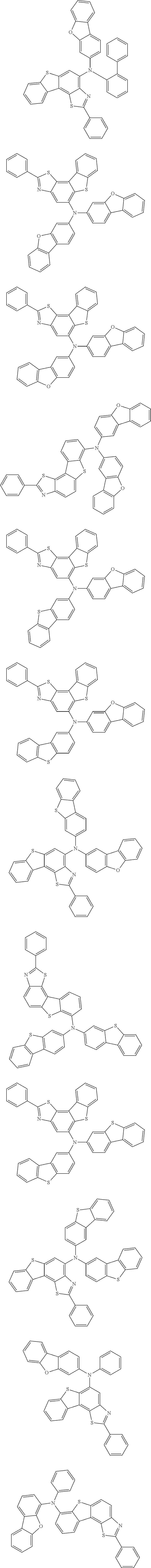

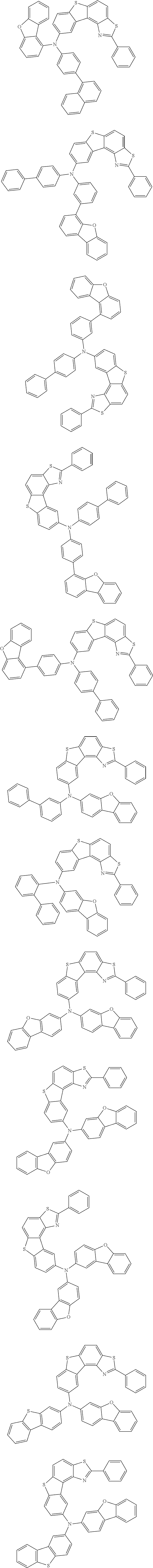

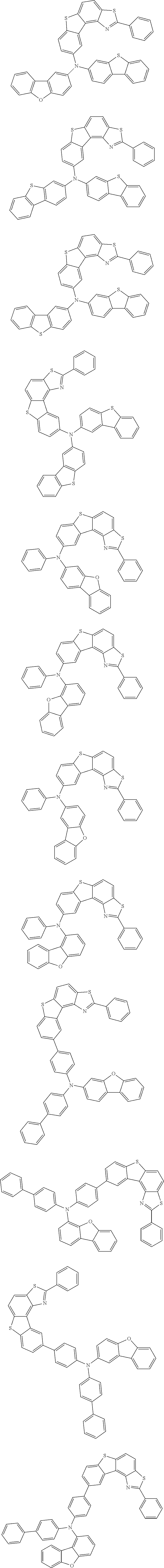

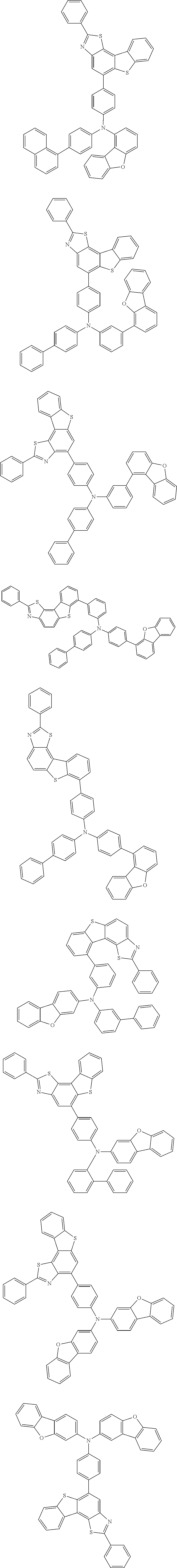

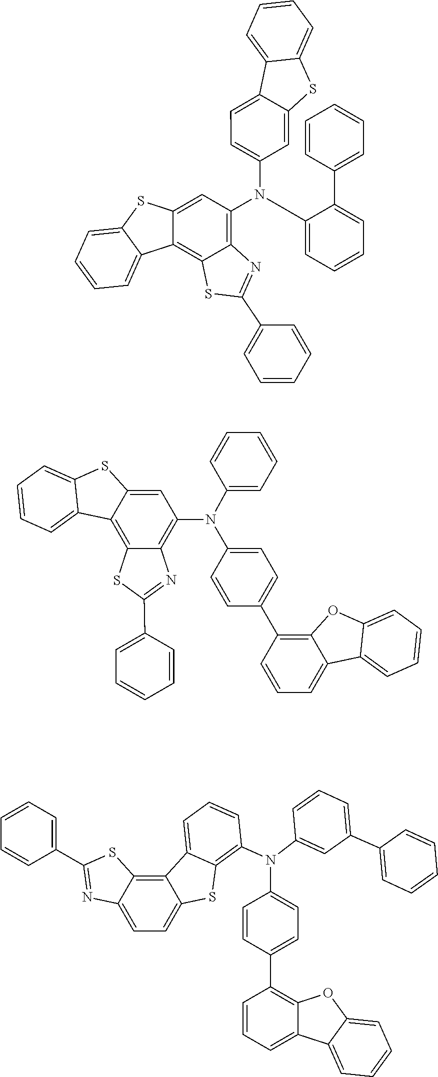 [SMILES: c1ccc(-c2nc3c(N(c4ccc5c(c4)sc4ccccc45)c4ccccc4-c4ccccc4)cc4sc5ccccc5c4c3s2)cc1 ; c1ccc(-c2nc3c(N(c4ccccc4)c4ccc(-c5cccc6c5oc5ccccc56)cc4)cc4sc5ccccc5c4c3s2)cc1 ; c1ccc(-c2cccc(N(c3ccc(-c4cccc5c4oc4ccccc45)cc3)c3cccc4c3sc3ccc5nc(-c6ccccc6)sc5c34)c2)cc1]

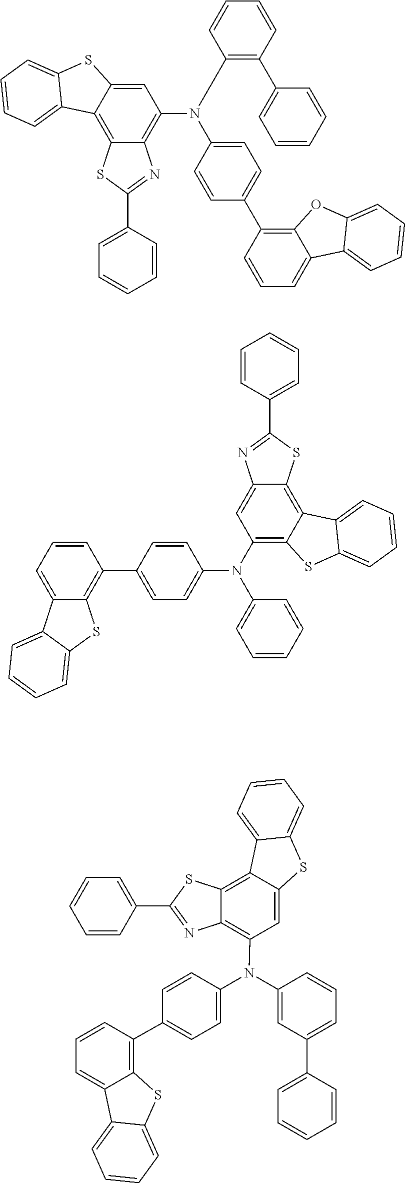 [SMILES: c1ccc(-c2nc3c(N(c4ccc(-c5cccc6c5oc5ccccc56)cc4)c4ccccc4-c4ccccc4)cc4sc5ccccc5c4c3s2)cc1 ; c1ccc(-c2nc3cc(N(c4ccccc4)c4ccc(-c5cccc6c5sc5ccccc56)cc4)c4sc5ccccc5c4c3s2)cc1 ; c1ccc(-c2cccc(N(c3ccc(-c4cccc5c4sc4ccccc45)cc3)c3cc4sc5ccccc5c4c4sc(-c5ccccc5)nc34)c2)cc1]

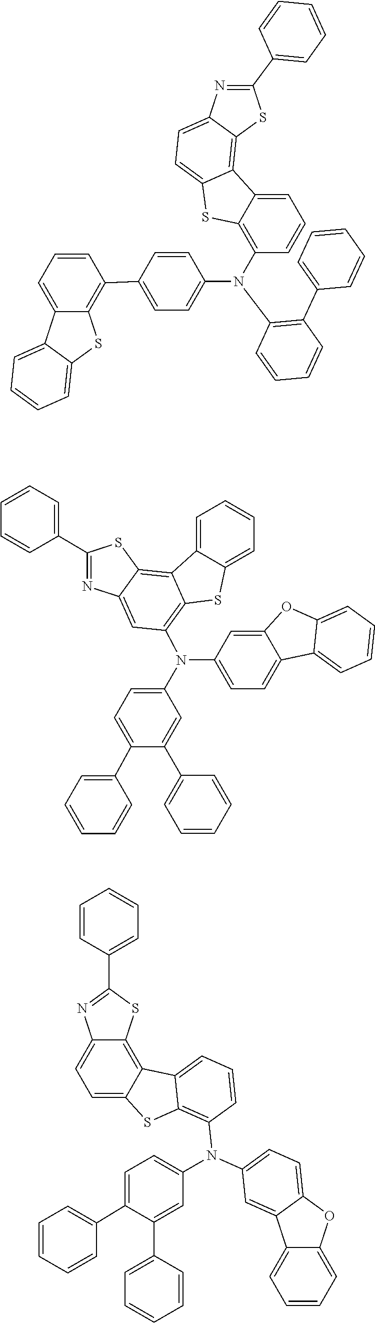 [SMILES: c1ccc(-c2nc3ccc4sc5c(N(c6ccc(-c7cccc8c7sc7ccccc78)cc6)c6ccccc6-c6ccccc6)cccc5c4c3s2)cc1 ; c1ccc(-c2nc3cc(N(c4ccc(-c5ccccc5)c(-c5ccccc5)c4)c4ccc5c(c4)oc4ccccc45)c4sc5ccccc5c4c3s2)cc1 ; c1ccc(-c2nc3ccc4sc5c(N(c6ccc(-c7ccccc7)c(-c7ccccc7)c6)c6ccc7oc8ccccc8c7c6)cccc5c4c3s2)cc1]

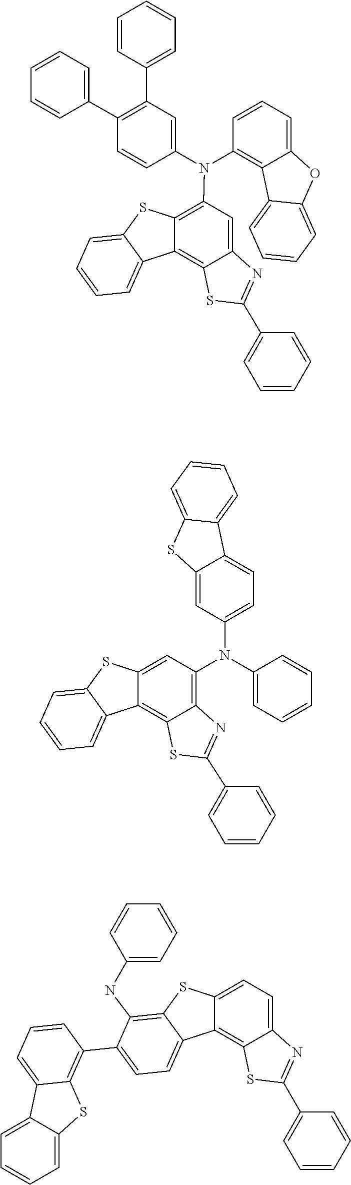 [SMILES: c1ccc(-c2nc3cc(N(c4ccc(-c5ccccc5)c(-c5ccccc5)c4)c4cccc5oc6ccccc6c45)c4sc5ccccc5c4c3s2)cc1 ; c1ccc(-c2nc3c(N(c4ccccc4)c4ccc5c(c4)sc4ccccc45)cc4sc5ccccc5c4c3s2)cc1 ; c1ccc(Nc2c(-c3cccc4c3sc3ccccc34)ccc3c2sc2ccc4nc(-c5ccccc5)sc4c23)cc1]

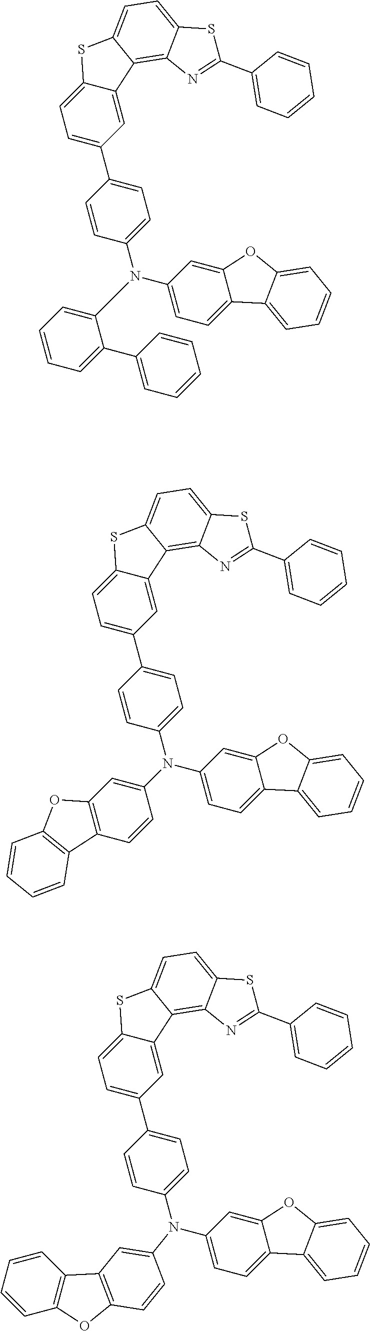 [SMILES: c1ccc(-c2nc3c(ccc4sc5ccc(-c6ccc(N(c7ccc8c(c7)oc7ccccc78)c7ccccc7-c7ccccc7)cc6)cc5c43)s2)cc1 ; c1ccc(-c2nc3c(ccc4sc5ccc(-c6ccc(N(c7ccc8c(c7)oc7ccccc78)c7ccc8c(c7)oc7ccccc78)cc6)cc5c43)s2)cc1 ; c1ccc(-c2nc3c(ccc4sc5ccc(-c6ccc(N(c7ccc8c(c7)oc7ccccc78)c7ccc8oc9ccccc9c8c7)cc6)cc5c43)s2)cc1]

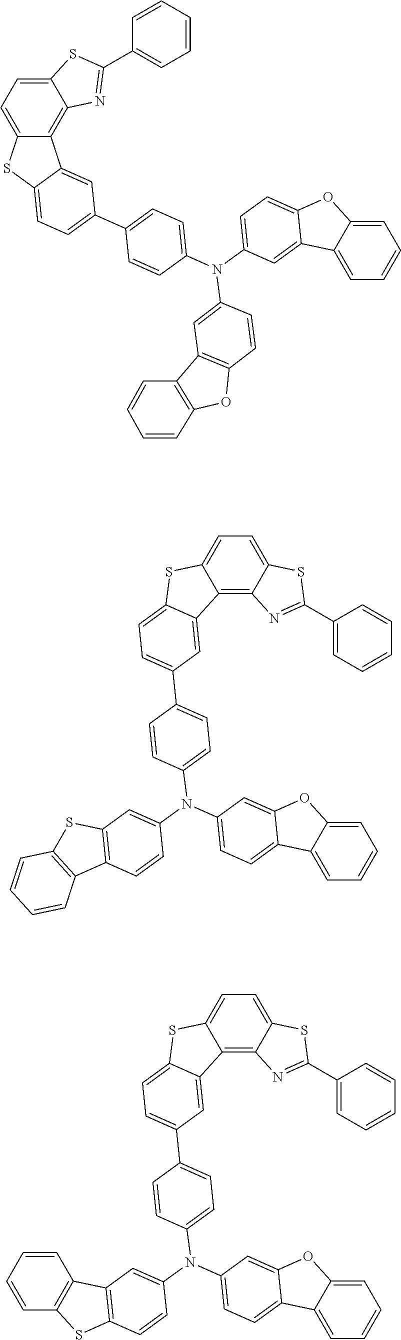 [SMILES: c1ccc(-c2nc3c(ccc4sc5ccc(-c6ccc(N(c7ccc8oc9ccccc9c8c7)c7ccc8oc9ccccc9c8c7)cc6)cc5c43)s2)cc1 ; c1ccc(-c2nc3c(ccc4sc5ccc(-c6ccc(N(c7ccc8c(c7)oc7ccccc78)c7ccc8c(c7)sc7ccccc78)cc6)cc5c43)s2)cc1 ; c1ccc(-c2nc3c(ccc4sc5ccc(-c6ccc(N(c7ccc8c(c7)oc7ccccc78)c7ccc8sc9ccccc9c8c7)cc6)cc5c43)s2)cc1]

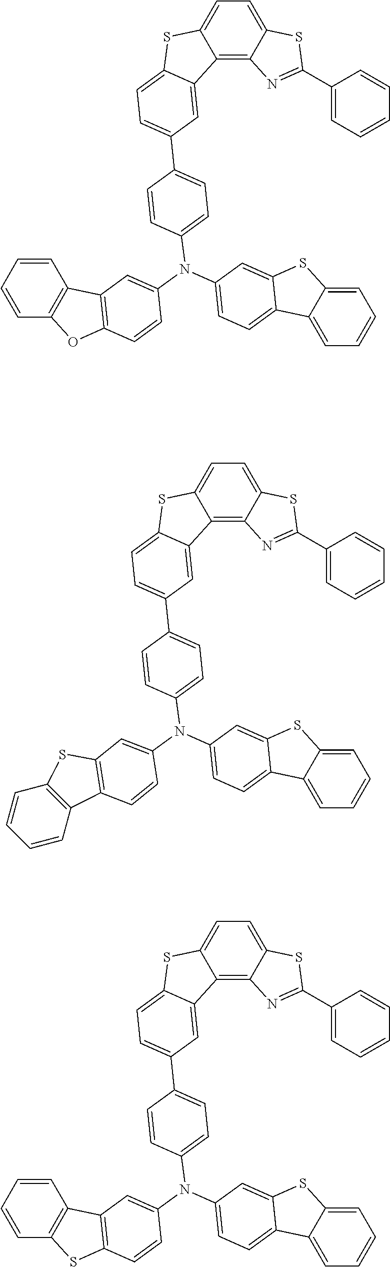 [SMILES: c1ccc(-c2nc3c(ccc4sc5ccc(-c6ccc(N(c7ccc8c(c7)sc7ccccc78)c7ccc8oc9ccccc9c8c7)cc6)cc5c43)s2)cc1 ; c1ccc(-c2nc3c(ccc4sc5ccc(-c6ccc(N(c7ccc8c(c7)sc7ccccc78)c7ccc8c(c7)sc7ccccc78)cc6)cc5c43)s2)cc1 ; c1ccc(-c2nc3c(ccc4sc5ccc(-c6ccc(N(c7ccc8c(c7)sc7ccccc78)c7ccc8sc9ccccc9c8c7)cc6)cc5c43)s2)cc1]

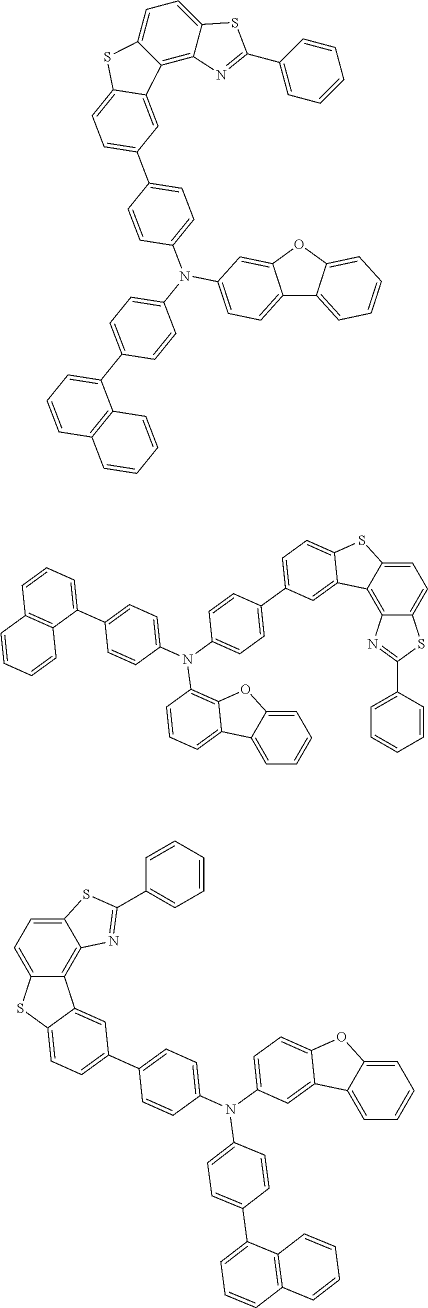 [SMILES: c1ccc(-c2nc3c(ccc4sc5ccc(-c6ccc(N(c7ccc(-c8cccc9ccccc89)cc7)c7ccc8c(c7)oc7ccccc78)cc6)cc5c43)s2)cc1 ; c1ccc(-c2nc3c(ccc4sc5ccc(-c6ccc(N(c7ccc(-c8cccc9ccccc89)cc7)c7cccc8c7oc7ccccc78)cc6)cc5c43)s2)cc1 ; c1ccc(-c2nc3c(ccc4sc5ccc(-c6ccc(N(c7ccc(-c8cccc9ccccc89)cc7)c7ccc8oc9ccccc9c8c7)cc6)cc5c43)s2)cc1]

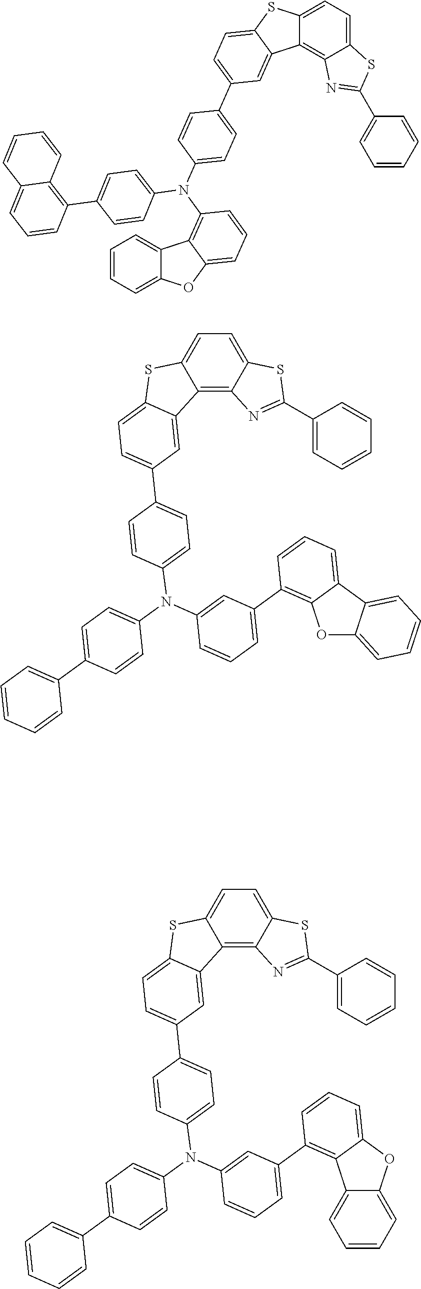 [SMILES: c1ccc(-c2nc3c(ccc4sc5ccc(-c6ccc(N(c7ccc(-c8cccc9ccccc89)cc7)c7cccc8oc9ccccc9c78)cc6)cc5c43)s2)cc1 ; c1ccc(-c2ccc(N(c3ccc(-c4ccc5sc6ccc7sc(-c8ccccc8)nc7c6c5c4)cc3)c3cccc(-c4cccc5c4oc4ccccc45)c3)cc2)cc1 ; c1ccc(-c2ccc(N(c3ccc(-c4ccc5sc6ccc7sc(-c8ccccc8)nc7c6c5c4)cc3)c3cccc(-c4cccc5oc6ccccc6c45)c3)cc2)cc1]

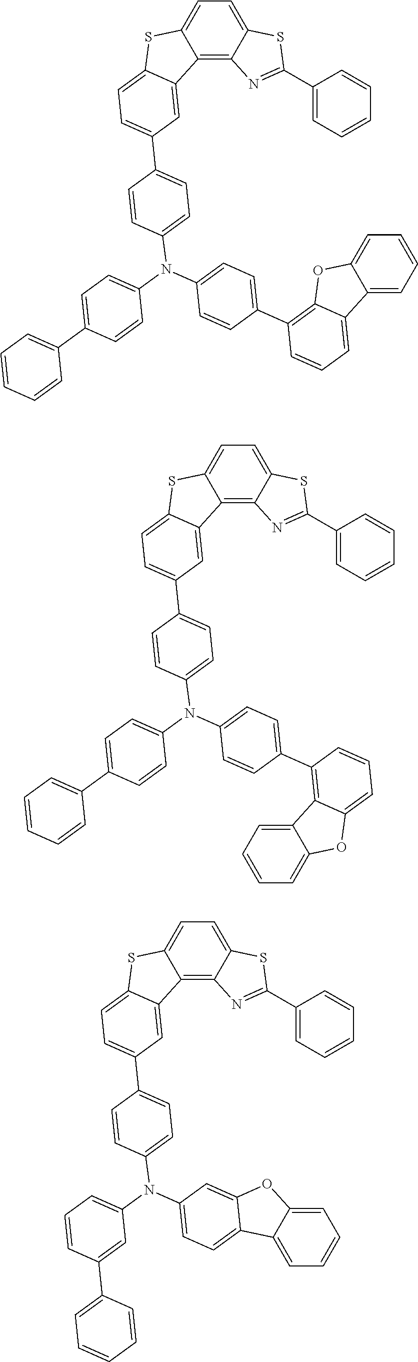 [SMILES: c1ccc(-c2ccc(N(c3ccc(-c4ccc5sc6ccc7sc(-c8ccccc8)nc7c6c5c4)cc3)c3ccc(-c4cccc5c4oc4ccccc45)cc3)cc2)cc1 ; c1ccc(-c2ccc(N(c3ccc(-c4ccc5sc6ccc7sc(-c8ccccc8)nc7c6c5c4)cc3)c3ccc(-c4cccc5oc6ccccc6c45)cc3)cc2)cc1 ; c1ccc(-c2cccc(N(c3ccc(-c4ccc5sc6ccc7sc(-c8ccccc8)nc7c6c5c4)cc3)c3ccc4c(c3)oc3ccccc34)c2)cc1]

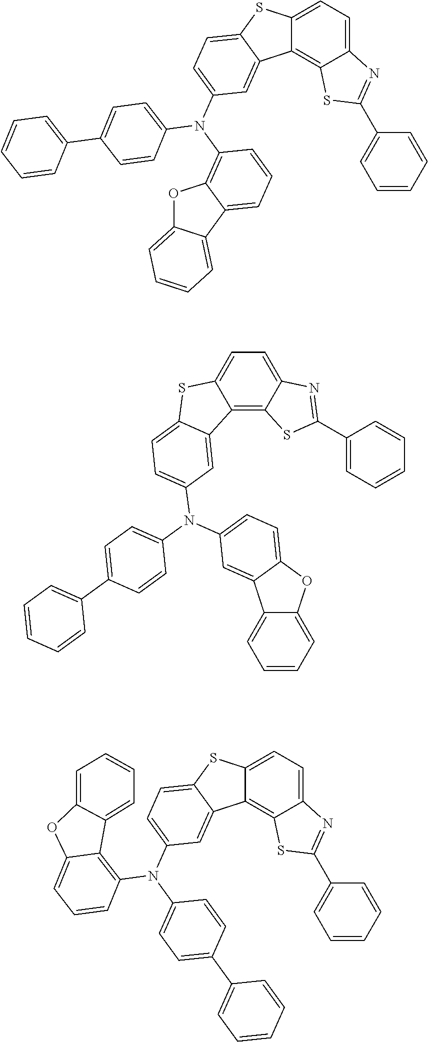 [SMILES: c1ccc(-c2ccc(N(c3ccc4sc5ccc6nc(-c7ccccc7)sc6c5c4c3)c3cccc4c3oc3ccccc34)cc2)cc1 ; c1ccc(-c2ccc(N(c3ccc4oc5ccccc5c4c3)c3ccc4sc5ccc6nc(-c7ccccc7)sc6c5c4c3)cc2)cc1 ; c1ccc(-c2ccc(N(c3ccc4sc5ccc6nc(-c7ccccc7)sc6c5c4c3)c3cccc4oc5ccccc5c34)cc2)cc1]

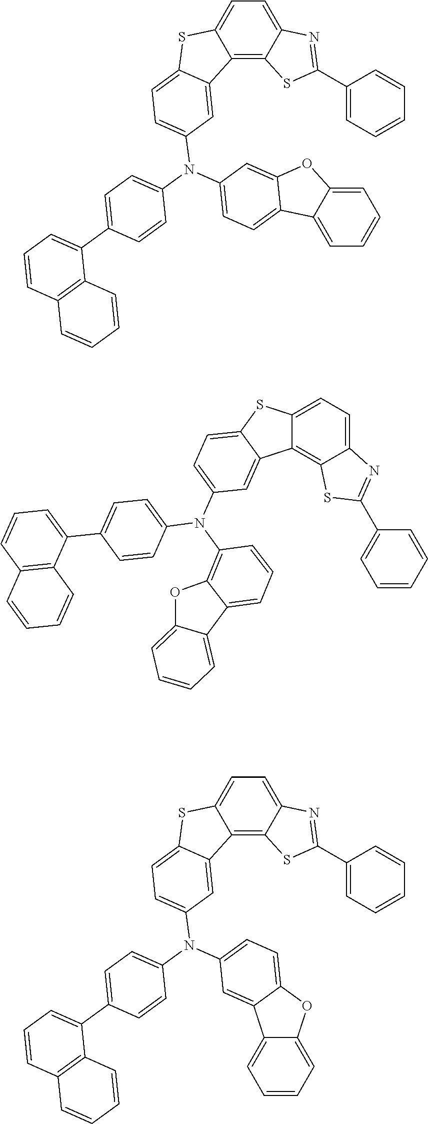 [SMILES: c1ccc(-c2nc3ccc4sc5ccc(N(c6ccc(-c7cccc8ccccc78)cc6)c6ccc7c(c6)oc6ccccc67)cc5c4c3s2)cc1 ; c1ccc(-c2nc3ccc4sc5ccc(N(c6ccc(-c7cccc8ccccc78)cc6)c6cccc7c6oc6ccccc67)cc5c4c3s2)cc1 ; c1ccc(-c2nc3ccc4sc5ccc(N(c6ccc(-c7cccc8ccccc78)cc6)c6ccc7oc8ccccc8c7c6)cc5c4c3s2)cc1]

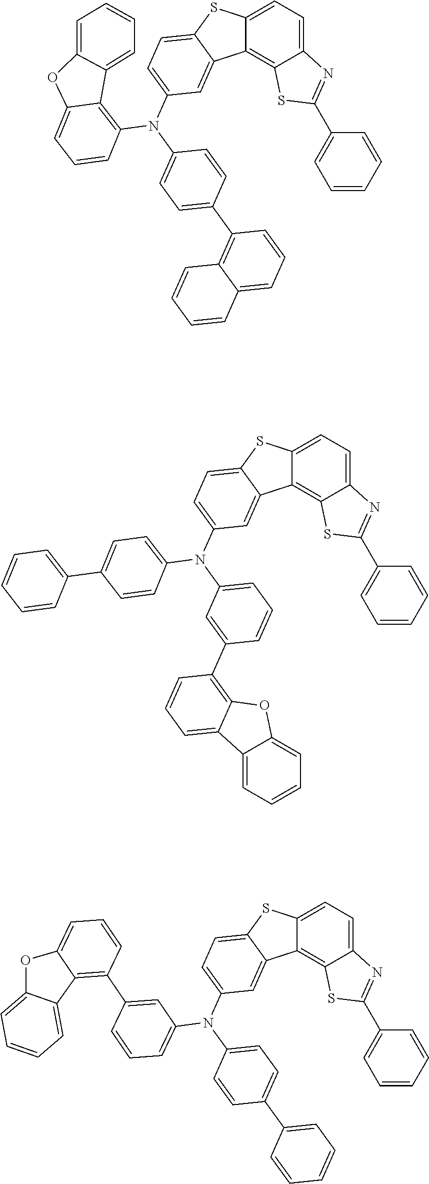 [SMILES: c1ccc(-c2nc3ccc4sc5ccc(N(c6ccc(-c7cccc8ccccc78)cc6)c6cccc7oc8ccccc8c67)cc5c4c3s2)cc1 ; c1ccc(-c2ccc(N(c3cccc(-c4cccc5c4oc4ccccc45)c3)c3ccc4sc5ccc6nc(-c7ccccc7)sc6c5c4c3)cc2)cc1 ; c1ccc(-c2ccc(N(c3cccc(-c4cccc5oc6ccccc6c45)c3)c3ccc4sc5ccc6nc(-c7ccccc7)sc6c5c4c3)cc2)cc1]

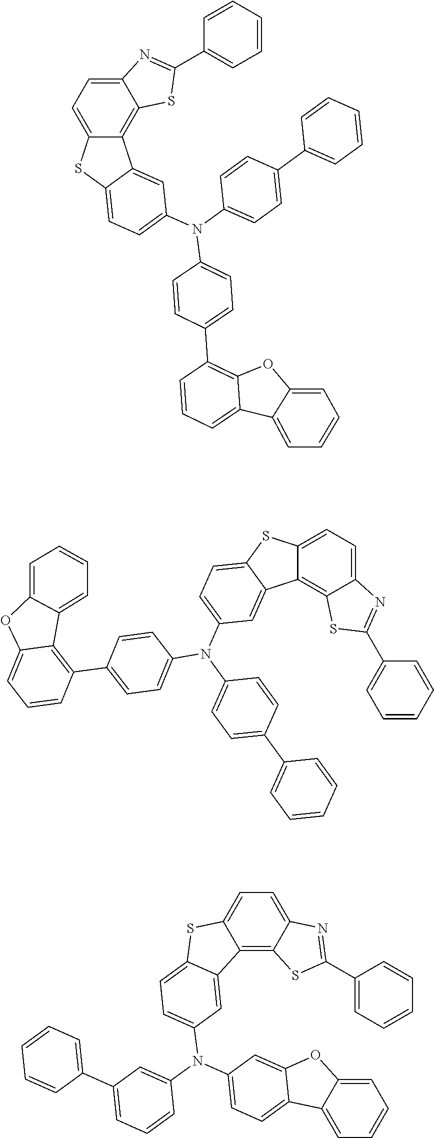 [SMILES: c1ccc(-c2ccc(N(c3ccc(-c4cccc5c4oc4ccccc45)cc3)c3ccc4sc5ccc6nc(-c7ccccc7)sc6c5c4c3)cc2)cc1 ; c1ccc(-c2ccc(N(c3ccc(-c4cccc5oc6ccccc6c45)cc3)c3ccc4sc5ccc6nc(-c7ccccc7)sc6c5c4c3)cc2)cc1 ; c1ccc(-c2cccc(N(c3ccc4c(c3)oc3ccccc34)c3ccc4sc5ccc6nc(-c7ccccc7)sc6c5c4c3)c2)cc1]

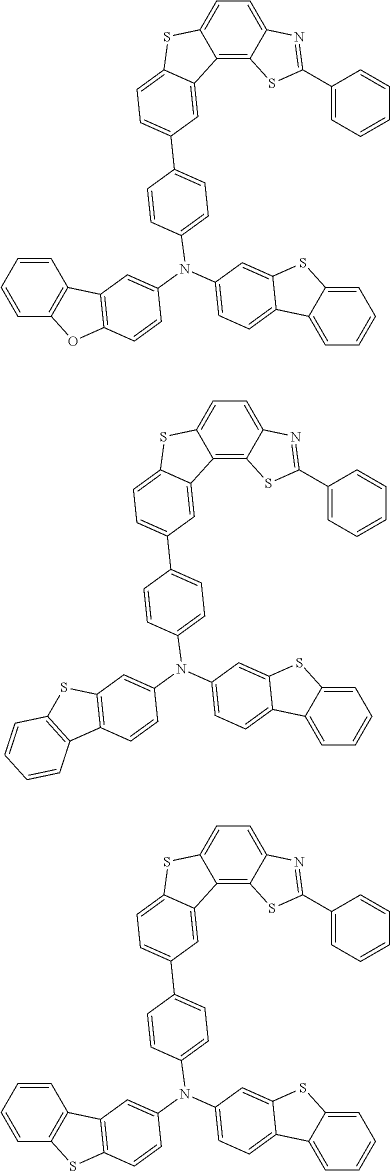 [SMILES: c1ccc(-c2nc3ccc4sc5ccc(-c6ccc(N(c7ccc8c(c7)sc7ccccc78)c7ccc8oc9ccccc9c8c7)cc6)cc5c4c3s2)cc1 ; c1ccc(-c2nc3ccc4sc5ccc(-c6ccc(N(c7ccc8c(c7)sc7ccccc78)c7ccc8c(c7)sc7ccccc78)cc6)cc5c4c3s2)cc1 ; c1ccc(-c2nc3ccc4sc5ccc(-c6ccc(N(c7ccc8c(c7)sc7ccccc78)c7ccc8sc9ccccc9c8c7)cc6)cc5c4c3s2)cc1]

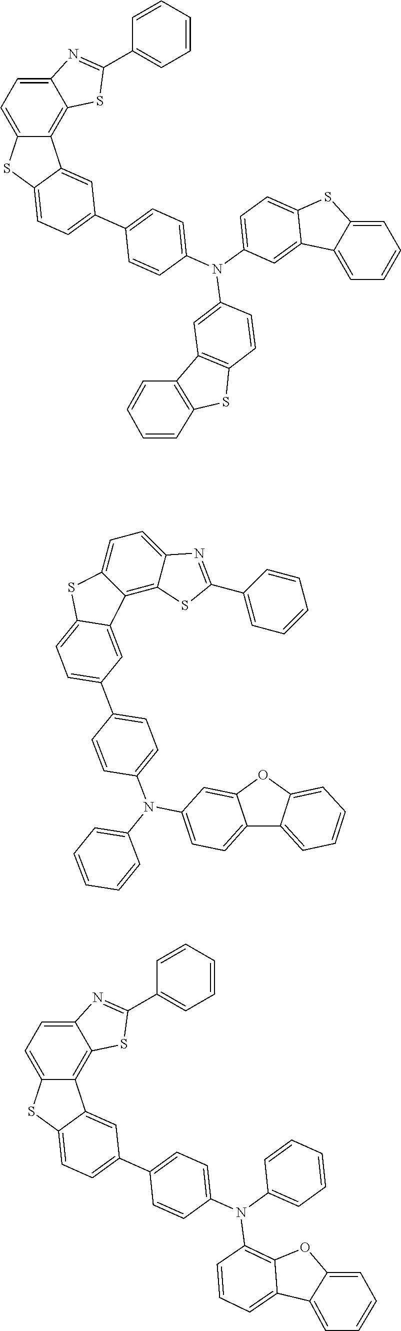 [SMILES: c1ccc(-c2nc3ccc4sc5ccc(-c6ccc(N(c7ccc8sc9ccccc9c8c7)c7ccc8sc9ccccc9c8c7)cc6)cc5c4c3s2)cc1 ; c1ccc(-c2nc3ccc4sc5ccc(-c6ccc(N(c7ccccc7)c7ccc8c(c7)oc7ccccc78)cc6)cc5c4c3s2)cc1 ; c1ccc(-c2nc3ccc4sc5ccc(-c6ccc(N(c7ccccc7)c7cccc8c7oc7ccccc78)cc6)cc5c4c3s2)cc1]

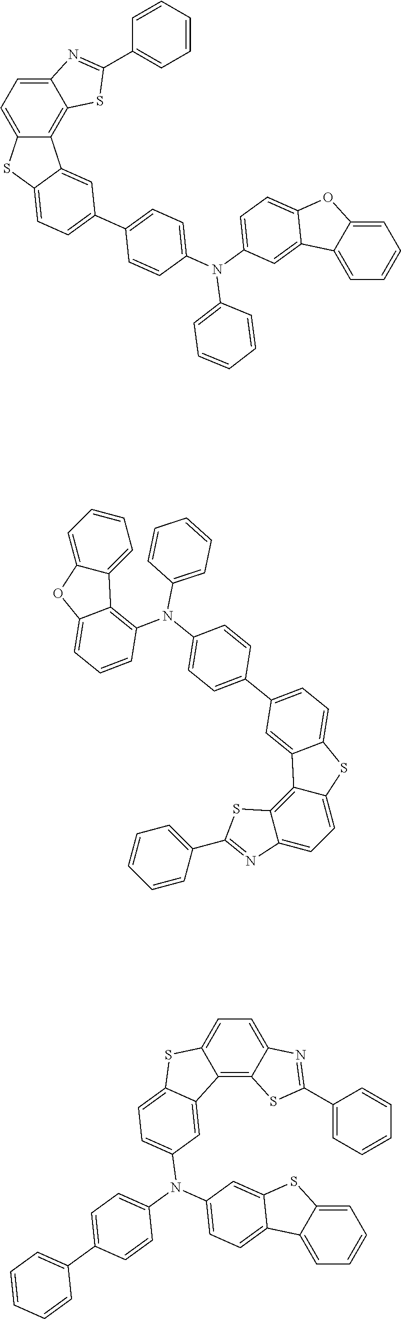 [SMILES: c1ccc(-c2nc3ccc4sc5ccc(-c6ccc(N(c7ccccc7)c7ccc8oc9ccccc9c8c7)cc6)cc5c4c3s2)cc1 ; c1ccc(-c2nc3ccc4sc5ccc(-c6ccc(N(c7ccccc7)c7cccc8oc9ccccc9c78)cc6)cc5c4c3s2)cc1 ; c1ccc(-c2ccc(N(c3ccc4c(c3)sc3ccccc34)c3ccc4sc5ccc6nc(-c7ccccc7)sc6c5c4c3)cc2)cc1]

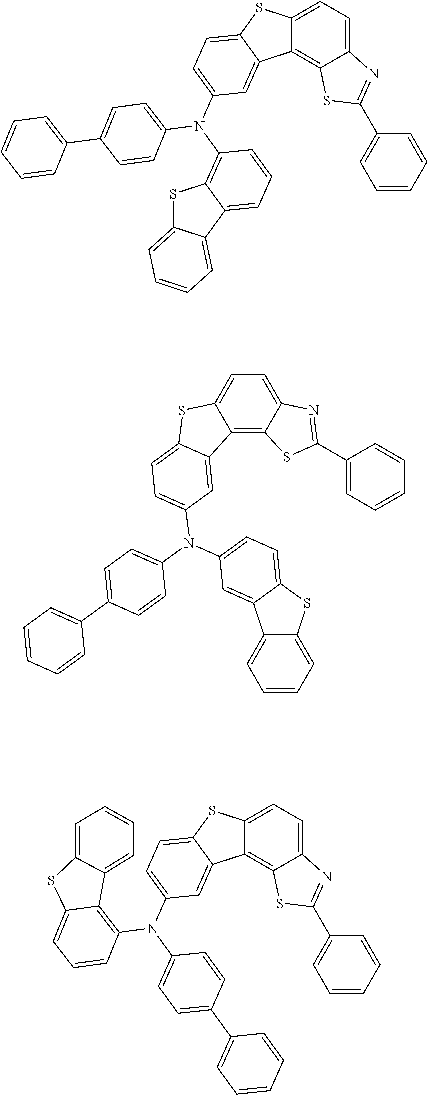 [SMILES: c1ccc(-c2ccc(N(c3ccc4sc5ccc6nc(-c7ccccc7)sc6c5c4c3)c3cccc4c3sc3ccccc34)cc2)cc1 ; c1ccc(-c2ccc(N(c3ccc4sc5ccccc5c4c3)c3ccc4sc5ccc6nc(-c7ccccc7)sc6c5c4c3)cc2)cc1 ; c1ccc(-c2ccc(N(c3ccc4sc5ccc6nc(-c7ccccc7)sc6c5c4c3)c3cccc4sc5ccccc5c34)cc2)cc1]

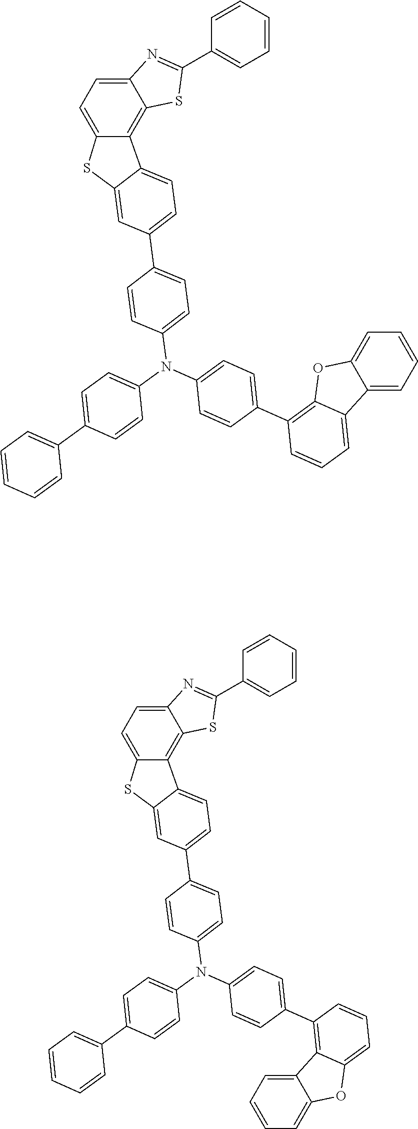 [SMILES: c1ccc(-c2ccc(N(c3ccc(-c4ccc5c(c4)sc4ccc6nc(-c7ccccc7)sc6c45)cc3)c3ccc(-c4cccc5c4oc4ccccc45)cc3)cc2)cc1 ; c1ccc(-c2ccc(N(c3ccc(-c4ccc5c(c4)sc4ccc6nc(-c7ccccc7)sc6c45)cc3)c3ccc(-c4cccc5oc6ccccc6c45)cc3)cc2)cc1]

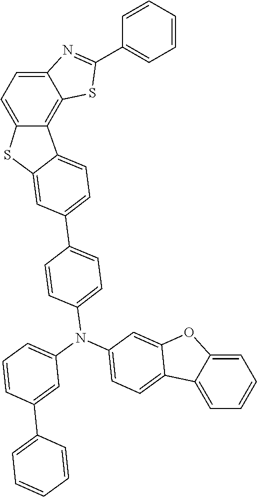 [SMILES: c1ccc(-c2cccc(N(c3ccc(-c4ccc5c(c4)sc4ccc6nc(-c7ccccc7)sc6c45)cc3)c3ccc4c(c3)oc3ccccc34)c2)cc1]

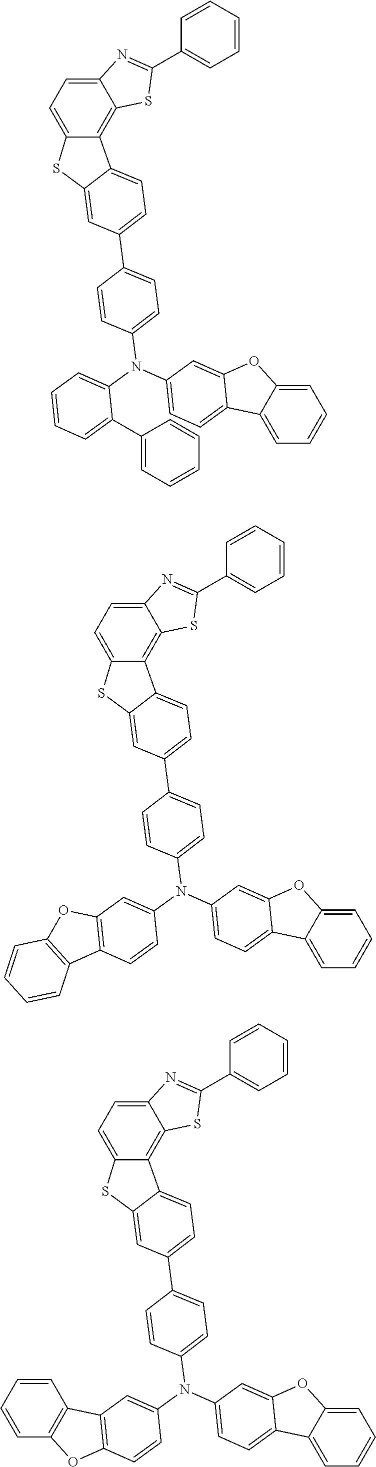 [SMILES: c1ccc(-c2nc3ccc4sc5cc(-c6ccc(N(c7ccc8c(c7)oc7ccccc78)c7ccccc7-c7ccccc7)cc6)ccc5c4c3s2)cc1 ; c1ccc(-c2nc3ccc4sc5cc(-c6ccc(N(c7ccc8c(c7)oc7ccccc78)c7ccc8c(c7)oc7ccccc78)cc6)ccc5c4c3s2)cc1 ; c1ccc(-c2nc3ccc4sc5cc(-c6ccc(N(c7ccc8c(c7)oc7ccccc78)c7ccc8oc9ccccc9c8c7)cc6)ccc5c4c3s2)cc1]

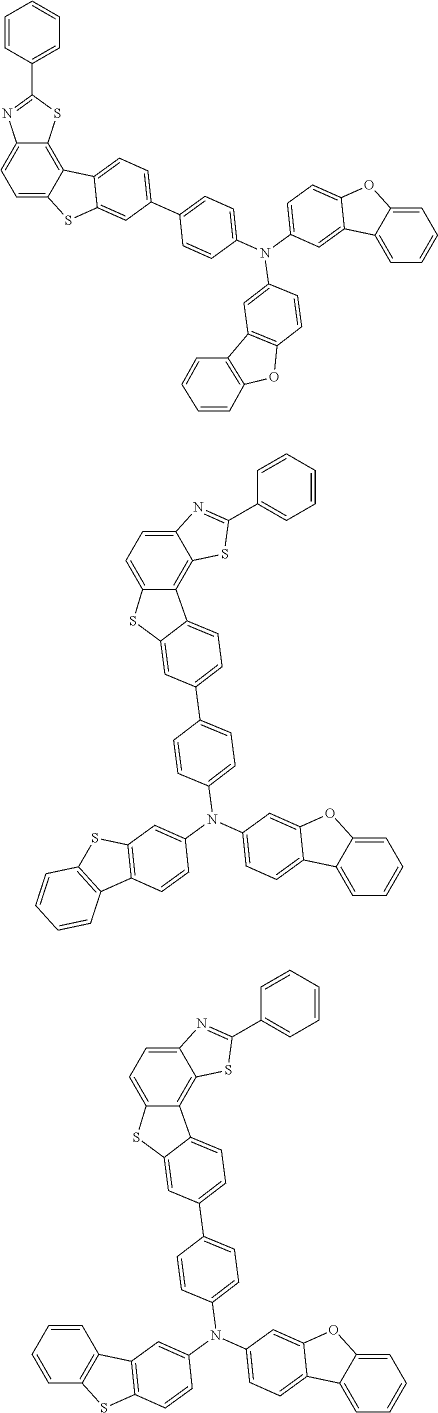 [SMILES: c1ccc(-c2nc3ccc4sc5cc(-c6ccc(N(c7ccc8oc9ccccc9c8c7)c7ccc8oc9ccccc9c8c7)cc6)ccc5c4c3s2)cc1 ; c1ccc(-c2nc3ccc4sc5cc(-c6ccc(N(c7ccc8c(c7)oc7ccccc78)c7ccc8c(c7)sc7ccccc78)cc6)ccc5c4c3s2)cc1 ; c1ccc(-c2nc3ccc4sc5cc(-c6ccc(N(c7ccc8c(c7)oc7ccccc78)c7ccc8sc9ccccc9c8c7)cc6)ccc5c4c3s2)cc1]

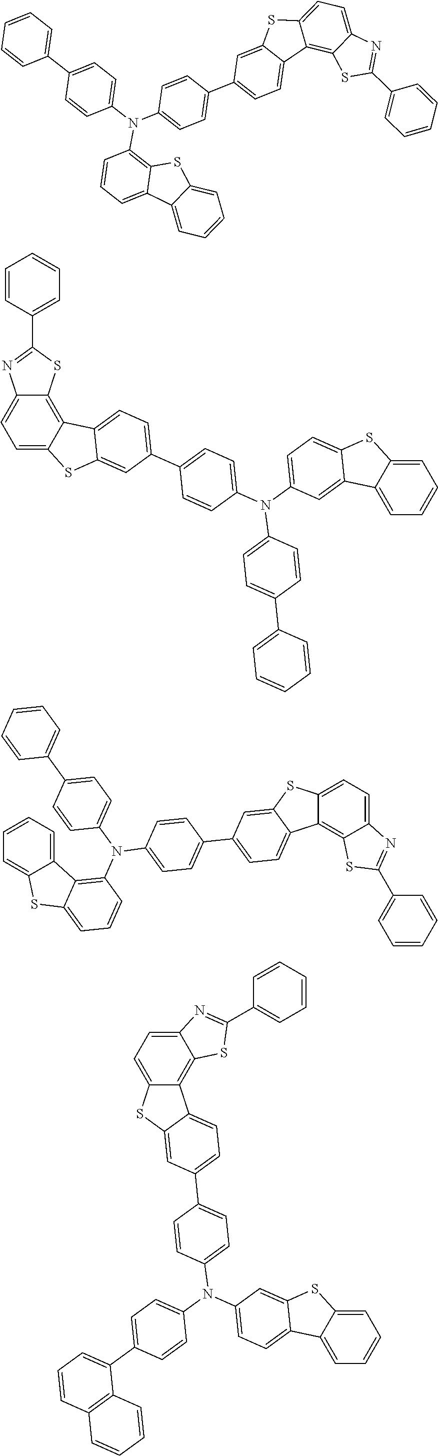 [SMILES: c1ccc(-c2ccc(N(c3ccc(-c4ccc5c(c4)sc4ccc6nc(-c7ccccc7)sc6c45)cc3)c3cccc4c3sc3ccccc34)cc2)cc1 ; c1ccc(-c2ccc(N(c3ccc(-c4ccc5c(c4)sc4ccc6nc(-c7ccccc7)sc6c45)cc3)c3ccc4sc5ccccc5c4c3)cc2)cc1 ; c1ccc(-c2ccc(N(c3ccc(-c4ccc5c(c4)sc4ccc6nc(-c7ccccc7)sc6c45)cc3)c3cccc4sc5ccccc5c34)cc2)cc1 ; c1ccc(-c2nc3ccc4sc5cc(-c6ccc(N(c7ccc(-c8cccc9ccccc89)cc7)c7ccc8c(c7)sc7ccccc78)cc6)ccc5c4c3s2)cc1]

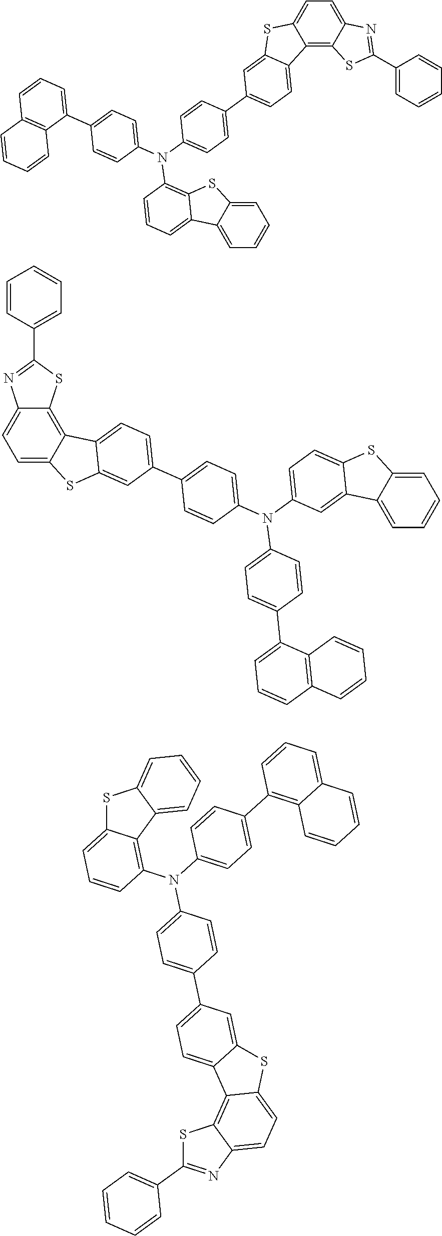

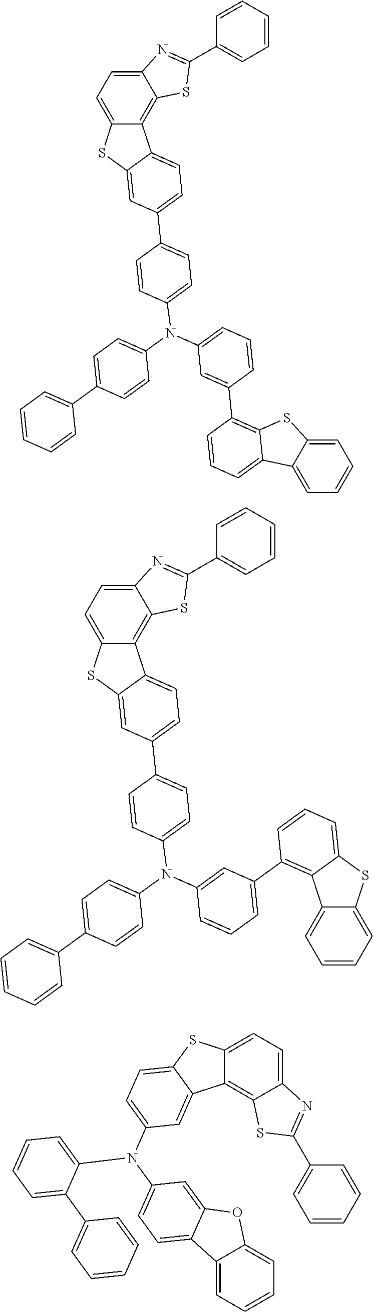

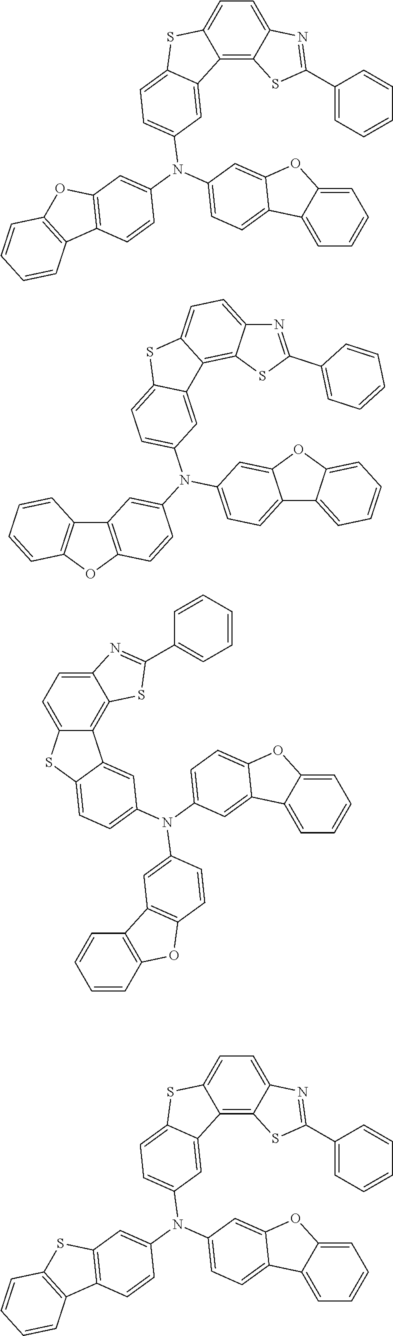 [SMILES: c1ccc(-c2nc3ccc4sc5ccc(N(c6ccc7c(c6)oc6ccccc67)c6ccc7c(c6)oc6ccccc67)cc5c4c3s2)cc1 ; c1ccc(-c2nc3ccc4sc5ccc(N(c6ccc7c(c6)oc6ccccc67)c6ccc7oc8ccccc8c7c6)cc5c4c3s2)cc1 ; c1ccc(-c2nc3ccc4sc5ccc(N(c6ccc7oc8ccccc8c7c6)c6ccc7oc8ccccc8c7c6)cc5c4c3s2)cc1 ; c1ccc(-c2nc3ccc4sc5ccc(N(c6ccc7c(c6)oc6ccccc67)c6ccc7c(c6)sc6ccccc67)cc5c4c3s2)cc1]

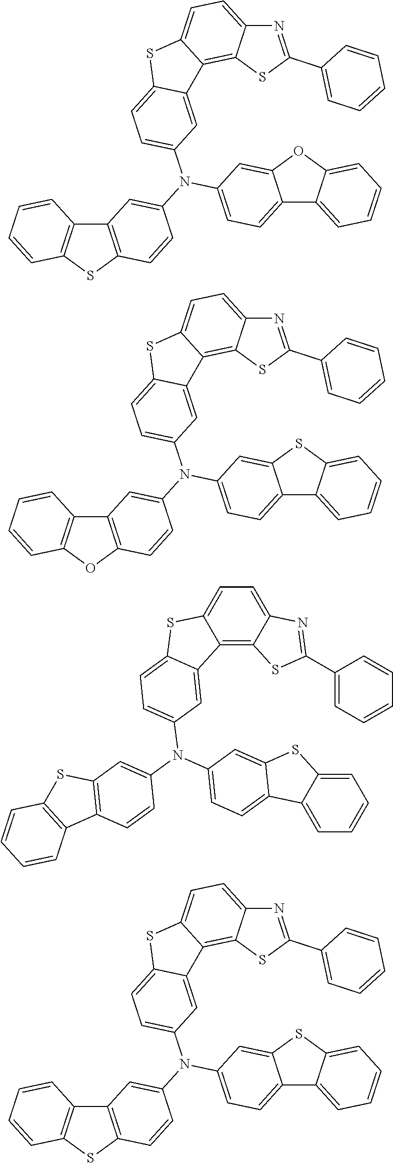 [SMILES: c1ccc(-c2nc3ccc4sc5ccc(N(c6ccc7c(c6)oc6ccccc67)c6ccc7sc8ccccc8c7c6)cc5c4c3s2)cc1 ; c1ccc(-c2nc3ccc4sc5ccc(N(c6ccc7c(c6)sc6ccccc67)c6ccc7oc8ccccc8c7c6)cc5c4c3s2)cc1 ; c1ccc(-c2nc3ccc4sc5ccc(N(c6ccc7c(c6)sc6ccccc67)c6ccc7c(c6)sc6ccccc67)cc5c4c3s2)cc1 ; c1ccc(-c2nc3ccc4sc5ccc(N(c6ccc7c(c6)sc6ccccc67)c6ccc7sc8ccccc8c7c6)cc5c4c3s2)cc1]

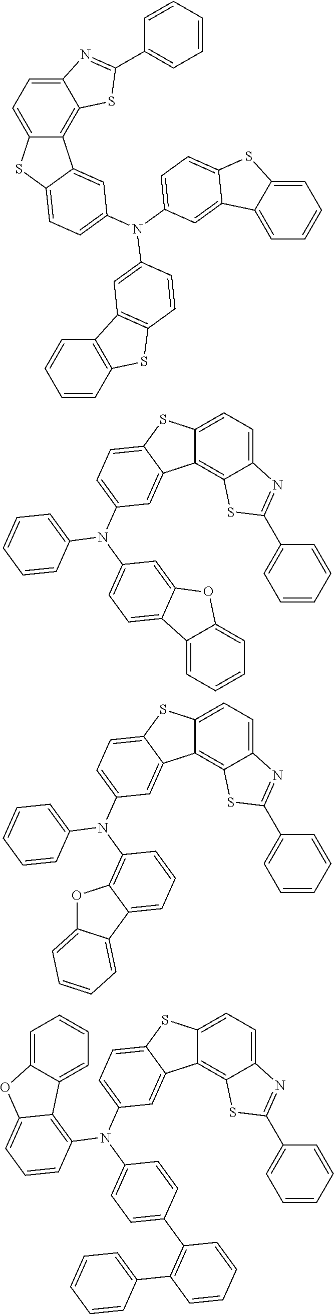

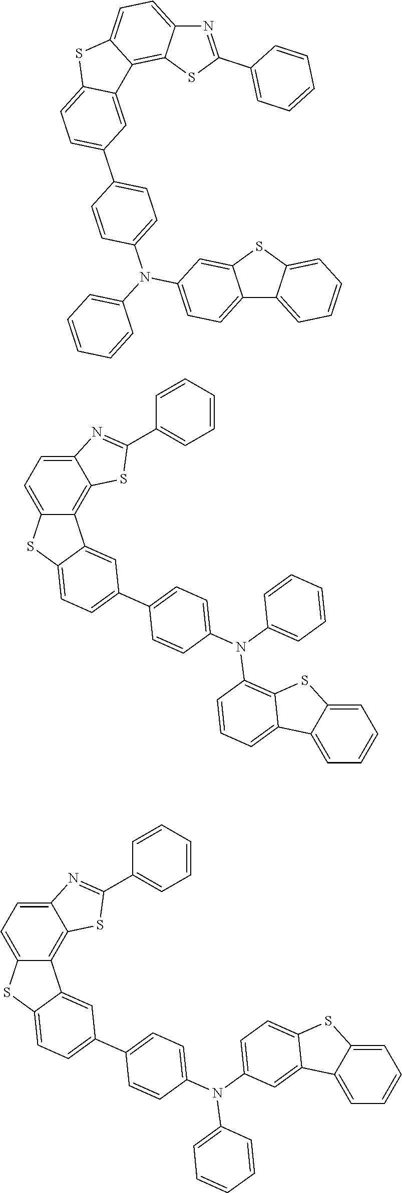 [SMILES: c1ccc(-c2nc3ccc4sc5ccc(-c6ccc(N(c7ccccc7)c7ccc8c(c7)sc7ccccc78)cc6)cc5c4c3s2)cc1 ; c1ccc(-c2nc3ccc4sc5ccc(-c6ccc(N(c7ccccc7)c7cccc8c7sc7ccccc78)cc6)cc5c4c3s2)cc1 ; c1ccc(-c2nc3ccc4sc5ccc(-c6ccc(N(c7ccccc7)c7ccc8sc9ccccc9c8c7)cc6)cc5c4c3s2)cc1]

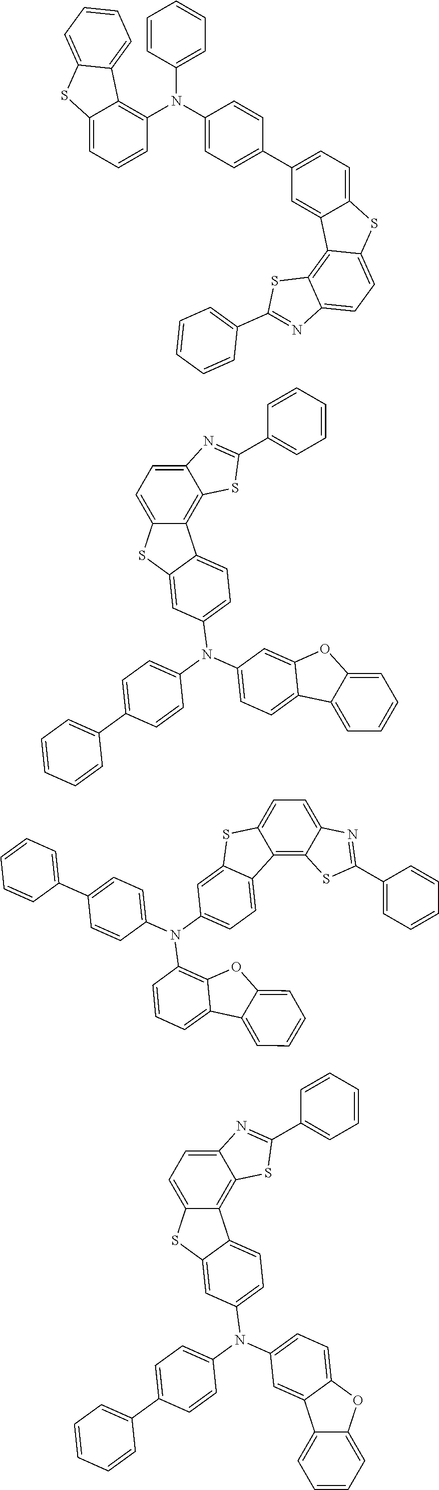

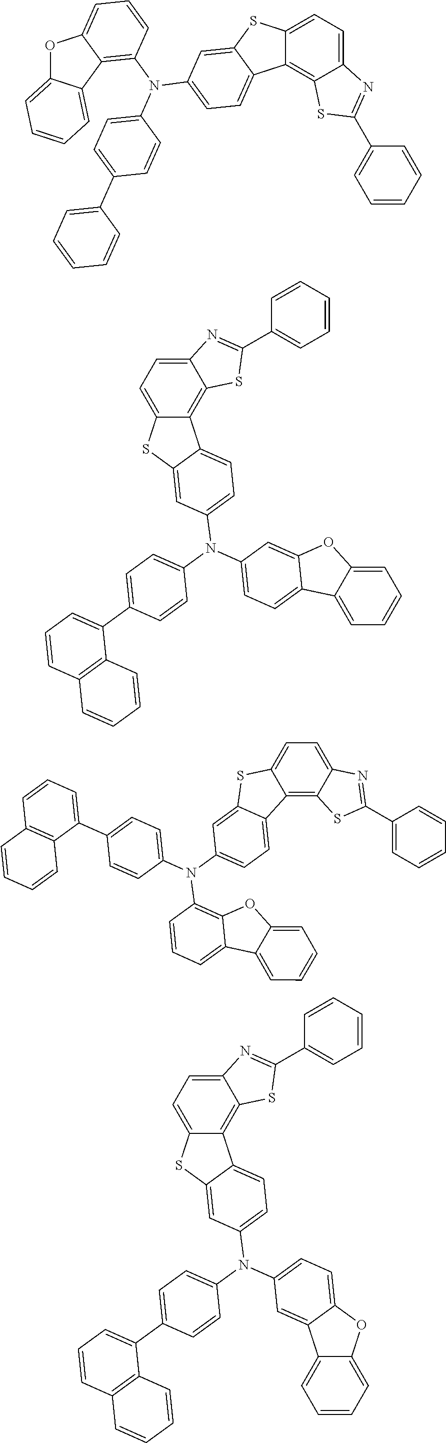

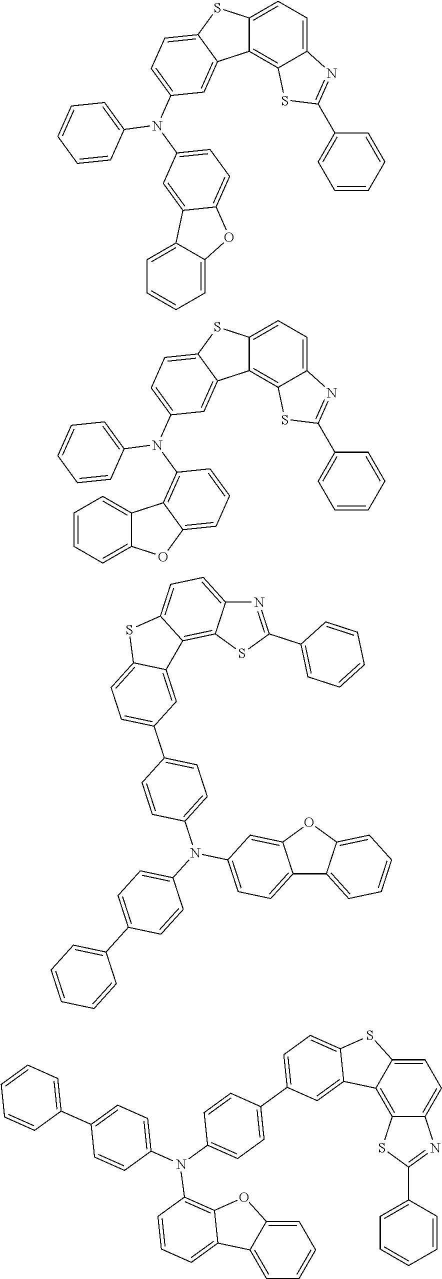

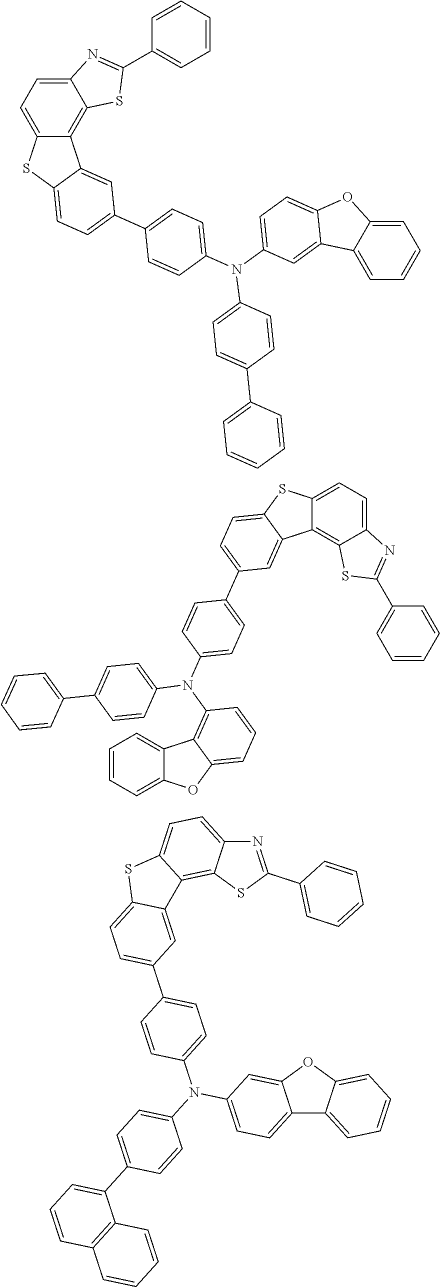

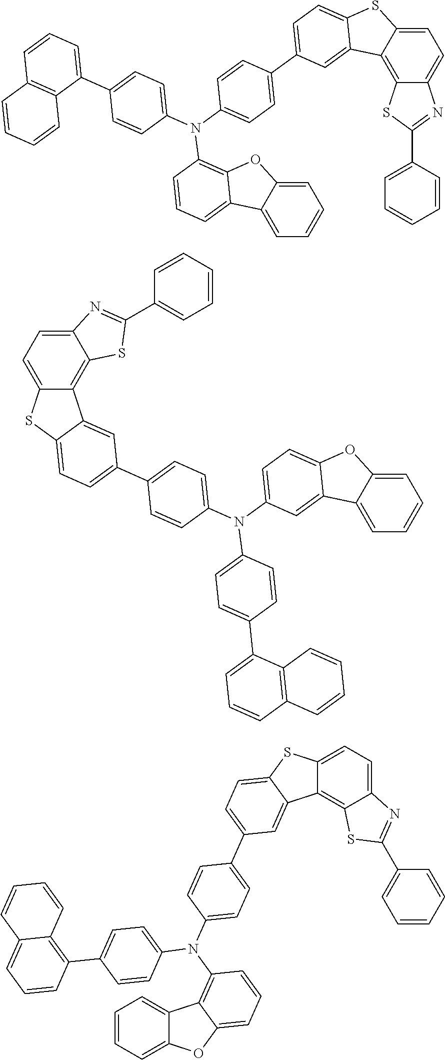

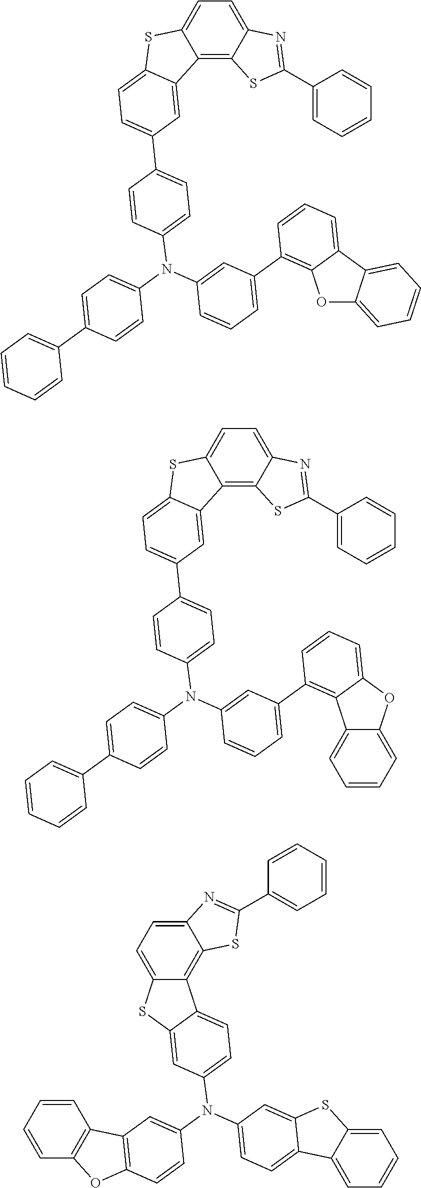 [SMILES: c1ccc(-c2ccc(N(c3ccc(-c4ccc5sc6ccc7nc(-c8ccccc8)sc7c6c5c4)cc3)c3cccc(-c4cccc5c4oc4ccccc45)c3)cc2)cc1 ; c1ccc(-c2ccc(N(c3ccc(-c4ccc5sc6ccc7nc(-c8ccccc8)sc7c6c5c4)cc3)c3cccc(-c4cccc5oc6ccccc6c45)c3)cc2)cc1 ; c1ccc(-c2nc3ccc4sc5cc(N(c6ccc7c(c6)sc6ccccc67)c6ccc7oc8ccccc8c7c6)ccc5c4c3s2)cc1]

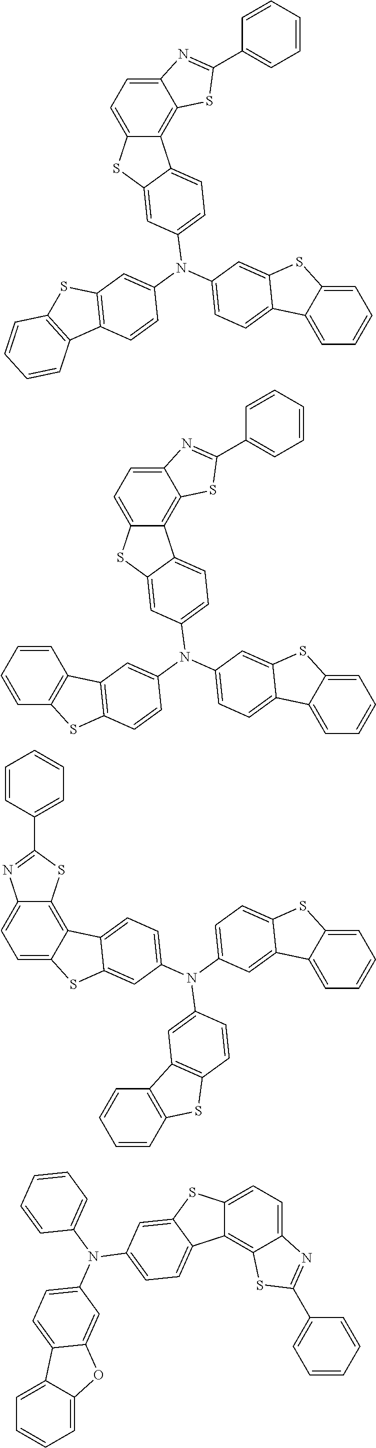

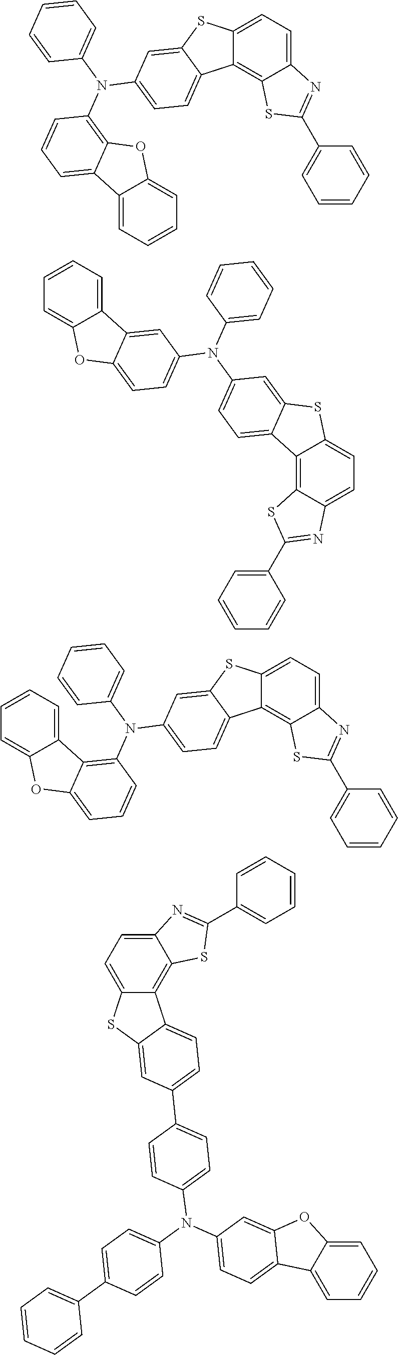

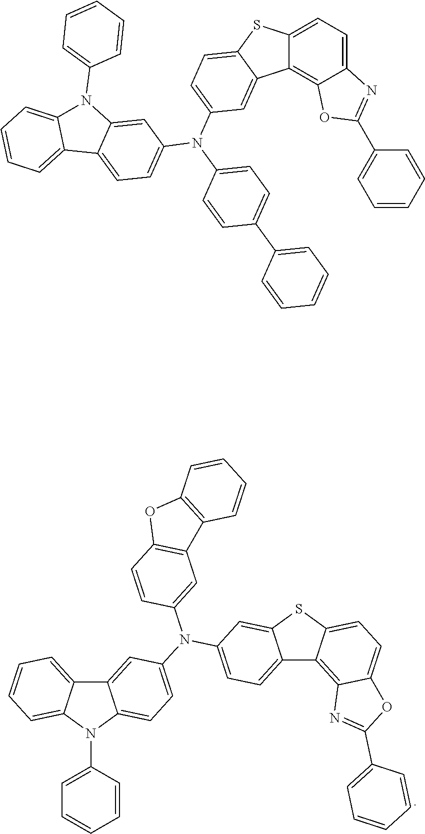 [SMILES: c1ccc(-c2ccc(N(c3ccc4sc5ccc6nc(-c7ccccc7)oc6c5c4c3)c3ccc4c5ccccc5n(-c5ccccc5)c4c3)cc2)cc1 ; c1ccc(-c2nc3c(ccc4sc5cc(N(c6ccc7oc8ccccc8c7c6)c6ccc7c(c6)c6ccccc6n7-c6ccccc6)ccc5c43)o2)cc1]

9. An organic light emitting device, comprising:
a first electrode;
a second electrode that is opposite to the first electrode; and
one or more organic material layers that are between the first electrode and the second electrode, wherein at least one layer of the one or more organic material layers comprises the compound of claim 1.

10. The organic light emitting device of claim 9, wherein the at least one layer comprising the compound is a light emitting layer, an electron blocking layer, or both a light emitting layer and an electron blocking layer.
